(19) United States
(12) Patent Application Publication
(10) Pub. No.: US 2016/0010068 A1
(43) Pub. Date: Jan. 14, 2016

(54) FUSION POLYNUCLEOTIDES AND FUSION POLYPEPTIDES ASSOCIATED WITH CANCER AND PARTICULARLY MELANOMA AND THEIR USES AS THERAPEUTIC AND DIAGNOSTIC TARGETS

(71) Applicants: Boris C. BASTIAN, (US); Maureen CRONIN, (US); Jie HE, (US); Doron LIPSON, (US); Philip James STEPHENS, (US); Thomas WIESER, (US); Roman YALENSKY, (US)

(72) Inventors: Boris C. BASTIAN, San Francisco, CA (US); Maureen CRONIN, Cambridge, MA (US); Jie HE, Newton, MA (US); Doron LIPSON, Chestnut Hill, MA (US); James Philip STEPHENS, Lexington, MA (US); Thomas WIESER, New York, NY (US); Roman YALENSKY, Newton, MA (US)

(21) Appl. No.: 14/770,776

(22) PCT Filed: Feb. 24, 2014
(86) PCT No.: PCT/US2014/018120

§ 371 Date: Aug. 26, 2015

Related U.S. Application Data
(60) Provisional application No. 61/768,340, filed on Feb. 22, 2013.

(51) Int. Cl. C12N 9/12; C07K 16/40; C07K 14/47; C07K 14/74; C12Q 1/68; G01N 33/574

(52) U.S. Cl. CPC C12N 9/12; C12Q 1/6886; G01N 33/57496; C07K 14/47; C07K 14/70539; C07K 14/4702; C07K 14/4746; C07K 16/40; C12Q 2600/156; C12Y 207/10001; C12Y 207/11001; C07K 2319/00; C07K 2317/76

(57) ABSTRACT

Novel fusion molecules and uses are disclosed.

This application is being filed on Feb. 24, 2014, as a PCT International patent application, and claims priority to U.S. Provisional Patent Application No. 61/768,340, filed Feb. 22, 2013, the disclosure of which is incorporated by reference in its entirety.

### GOVERNMENT RIGHTS

This disclosure was funded in part by grants from the National Institutes of Health R01 CA131524; P01 CA025874; and P30 CA008748. The U.S. government has certain rights in this disclosure.

### SEQUENCE LISTING

The present application includes a Sequence Listing in electronic form as a txt file in ASCII format titled “60009_{—}0015WOU1SEQ_LIST_ST113.TXT” and having a size of 741 kb. The contents of this txt file are incorporated by reference herein.

### BACKGROUND

Cancer represents the phenotypic end-point of multiple genetic lesions that endow cells with a full range of biological properties required for tumorigenesis. Indeed, a hallmark genomic feature of many cancers, including, for example, B cell cancer, lung cancer, breast cancer, ovarian cancer, pancreatic cancer, and colon cancer, is the presence of numerous complex chromosome structural aberrations, including translocations, intra-chromosomal inversions, point mutations, deletions, gene copy number changes, gene expression level changes, and germline mutations, among others.

The need still exists for identifying novel genetic lesions associated with cancer. Such genetic lesions can be an effective approach to develop compositions, methods and assays for evaluating and treating cancer patients.

### SUMMARY

The invention is based, at least in part, on the discovery of novel rearrangement events that give rise to fusion molecules that includes a fragment of a first gene and a fragment of a second gene, e.g., a fusion that includes a 5′-exon and a 3′-exon summarized in FIGS. 1A-1C. The term “fusion” or “fusion molecule” is used generically herein, and includes any fusion molecule (e.g., gene, gene product (e.g., cDNA, mRNA, or polypeptide), and variant thereof) that includes a fragment of first gene and a fragment of second gene described herein, including, e.g., anTP53-NTRK1, CEP89-BRAF, CLIP1-ROS1, and so on summarized in FIGS. 1A-1C. Expression of the fusion molecules was detected in cancer tissues, thus suggesting an association with neoplastic growth or cancer (including pre-malignant, or malignant and/or metastatic growth).

Accordingly, the invention provides, at least in part, the following: methods for identifying, assessing or detecting a fusion molecule as described herein; methods for identifying, assessing, evaluating, and/or treating a subject having a cancer, e.g., a cancer having a translocation, manifest as a fusion molecule as described herein, particularly melanoma; isolated fusion nucleic acid molecules, nucleic acid constructs, host cells containing the nucleic acid molecules; purified fusion polypeptides and binding agents; detection reagents (e.g., probes, primers, antibodies, kits, capable. e.g., of specific detection of a fusion nucleic acid or protein); screening assays for identifying molecules that interact with, e.g., inhibit, the fusions, e.g., novel kinase inhibitors; as well as assays and kits for evaluating, identifying, assessing and/or treating a subject having a cancer, e.g., a cancer having a fusion particularly melanoma. The compositions and methods identified herein can be used, for example, to identify new inhibitors; to evaluate, identify or select a subject, e.g., a patient, having a cancer who is a candidate for treatment with these inhibitors; and to treat or prevent or postpone a cancer, such as a melanocytic neoplasm.

Each of these fusion molecules is described herein in more detail.

### CLIP1-ROS1 Fusions

In one embodiment, a fusion includes an in-frame fusion of an exon of CAP-GLY domain containing linker protein 1 (CLIP1), e.g., one more exons of CLIP1 (e.g., one or more of exons 1-20 of CLIP1) or a fragment thereof, and an exon of C-Ros oncogene 1 (ROS1), e.g., one or more exons of a ROS1 (e.g., one or more of exons 36-43 of ROS1) or a fragment thereof. For example, the CLIP1-ROS1 fusion can include an in-frame fusion within an intron of CLIP1 (e.g., intron 20) or a fragment thereof, with an intron of ROS1 (e.g., intron 35) or a fragment thereof. In one embodiment, the fusion of the CLIP1-ROS1 fusion comprises the nucleotide sequence of: chromosome 12 at one or more of a nucleotide (plus or minus 10, 20, 30, 50, 60, 70, 80, 100 or more nucleotides) and chromosome 6 at one or more of a nucleotide (plus or minus 10, 20, 30, 50, 60, 70, 80, 100 or more nucleotides). In one embodiment, the CLIP1-ROS1 fusion is a translocation, e.g., a translocation of a portion of chromosome 12 and a portion of chromosome 6.

In certain embodiments, the CLIP1-ROS1 fusion is in a 5′-CLIP1 to 3′-ROS1 configuration (also referred to herein as “5′-CLIP1-ROS1-3′).” The term “fusion” or “fusion molecule” can refer to a polypeptide or a nucleic acid fusion, depending on the context. It may include a full-length sequence of a fusion or a fragment thereof, e.g., a fusion junction (e.g., a fragment including a portion of CLIP1 and a portion of ROS1, e.g., a portion of the CLIP1-ROS1 fusion described herein). In one embodiment, the CLIP1-ROS1 fusion polypeptide includes a fragment of the amino acid sequence shown in SEQ ID NO:16 and a fragment of the amino acid sequence shown in SEQ ID NO: 12, or an amino acid sequence substantially identical thereto. In another embodiment, the CLIP1-ROS1 fusion nucleic acid includes a fragment of the nucleotide sequence shown in SEQ ID NO: 15 and a fragment of the nucleotide sequence shown in SEQ ID NO: 11, or a nucleotide sequence substantially identical thereto. In one embodiment, the CLIP1-ROS1 fusion polypeptide comprises sufficient CLIP1 and sufficient ROS1 sequence such that the 5′ CLIP1-3′ ROS1 fusion has kinase activity, e.g., has elevated activity, e.g., tyrosine kinase activity, as compared with wild type CLIP1 or ROS1. In any event, the fusion causes activation in the cells harboring of oncogenic signaling pathways.

In certain embodiments, the CLIP1-ROS1 fusion comprises one or more (or all of) exons 1-20 from CLIP1 and one or more (or all of) exons 36-43 of ROS1 (e.g., one or more of the exons shown in SEQ ID NO:15 and SEQ ID NO: 11. In another embodiment, the CLIP-ROS1 fusion comprises one or more (or all of) exons 1-20 of CLIP1 and one or more (or all of) exons 36-43 of ROS1. In certain embodiments, the CLIP1-ROS1 fusion comprises at least 1, 2, 3, 4, 5, 6, 7, 8, 9, 10, 11, 12, 13, 14, 15, 16, 17, 18, 19, 20 or more exons (or encoded exons) from CLIP1 and at least 1, 2, 3, 4, 5, 6, 7, 8 or more exons (or encoded exons) from ROS1 (e.g., from the CLIP1 and ROS1 sequences shown in SEQ ID NO:15 and SEQ ID NO:16 and SEQ ID NO: 11 and SEQ ID NO:12.

In certain embodiments, the CLIP1-ROS1 fusion comprises exons 1-20 or a fragment thereof from CLIP1, and exons 36-43 or a fragment thereof from ROS1 (e.g., as shown in SEQ ID NO: 15 and SEQ ID NO:11). In one embodiment, the CLIP1-ROS1 fusion comprises at least 5, 10, 15, 20, 30, 40, 50 or more amino acids from exons 1-20 of CLIP1 (e.g., from the amino acid sequence of CLIP1 as shown in SEQ ID NO:16 (e.g., from the amino acid sequence of CLIP1 preceding the fusion junction with ROS1, and at least 5, 10, 15, 20, 30, 40, 50 or more amino acids from exons 36-43 of ROS1 (e.g., from the amino acid sequence of ROS1 as shown in SEQ ID NO: 12). In another embodiment, the CLIP1-ROS1 fusion comprises at least 6, 12, 15, 20, 25, 50, 75, 100 or more nucleotides from exons 1-20 of CLIP1 (e.g., from the nucleotide sequence of CLIP1 as shown in SEQ ID NO:15 (e.g., from the nucleotide sequence of CLIP preceding the fusion junction with ROS1); and at least 6, 12, 15, 20, 25, 50, 75, 100 or more nucleotides from exons 36-43 of ROS1 (e.g., from the nucleotide sequence of ROS1 as shown in SEQ ID NO: 11).

In one embodiment, the CLIP1-ROS1 fusion includes the full sequence or a fragment of the nucleotide sequence shown in SEQ ID NO: 13 and SEQ ID NO:92, or a nucleotide sequence substantially identical thereto. In another embodiment, the CLIP1-ROS1 fusion polypeptide includes the full sequence or a fragment of the amino acid sequence shown in SEQ ID NO: 14 and SEQ ID NO:93, or an amino acid sequence substantially identical thereto.

### CLIP1-ROS1 Nucleic Acid Molecules

In one aspect, the invention features a nucleic acid molecule (e.g., an isolated or purified) nucleic acid molecule that includes a fragment of a CLIP1 gene and a fragment of a ROS1 gene. In one embodiment, the nucleotide sequence encodes a CLIP1-ROS1 fusion polypeptide that includes a tyrosine kinase domain or a functional fragment thereof. In another embodiment, the nucleotide sequence encodes a fragment of the ROS1 polypeptide including the amino acid sequence of SEQ ID NO: 12 or a fragment thereof, or a sequence substantially identical thereto. In other embodiments, the nucleic acid molecule includes a fragment of the CLIP1 gene encoding the amino acid sequence of SEQ ID NO: 16 or a fragment thereof, or a sequence substantially identical thereto. In yet other embodiments, the nucleic acid molecule includes a nucleotide sequence encoding the amino acid sequence shown in SEQ ID NO: 16, or a fragment thereof, and the amino acid sequence shown in SEQ ID NO:12 or a fragment thereof, or a sequence substantially identical thereto.

In one embodiment, the nucleic acid molecule includes a fusion, e.g., an in-frame fusion, between an intron of CLIP1 (e.g., intron 20, or a fragment thereof), and an intron of ROS1 (e.g., intron 35, or a fragment thereof). The CLIP1-ROS1 fusion can comprise a fusion of the nucleotide sequence of: chromosome 12 at one or more of a nucleotide (plus or minus 10, 20, 30, 50, 60, 70, 80, 100 nucleotides) and chromosome 6 at one or more of a nucleotide (plus or minus 10, 20, 30, 50, 60, 70, 80, 100 nucleotides), or a fragment thereof. In one embodiment, the CLIP1-ROS1 fusion comprises a fusion of the nucleotide sequence of: chromosome 12 at one or more of a nucleotide (plus or minus 10, 20, 30, 50, 60, 70, 80, 100 nucleotides) and chromosome 6 at one or more of a nucleotide (plus or minus 10, 20, 30, 50, 60, 70, 80, 100 nucleotides), or a fragment thereof.

In another embodiment, the CLIP1-ROS1 fusion comprises a nucleotide sequence (e.g., a fragment of a nucleotide sequence) shown in SEQ ID NO:15 and a nucleotide sequence (e.g., a fragment of a nucleotide sequence) shown in SEQ ID NO: 11, or a fragment of the fusion. In one embodiment, the CLIP1-ROS1 fusion comprises a nucleotide sequence substantially identical to the nucleotide sequence (e.g., a fragment of a nucleotide sequence) shown in SEQ ID NO: 15 and the nucleotide sequence (e.g., a fragment of a nucleotide sequence) shown SEQ ID NO: 11, or a fragment of the fusion. In one embodiment, the CLIP1-ROS1 fusion comprises a nucleotide sequence at least 70%, at least 75%, at least 80%, at least 85%, at least 90%, at least 95%, at least 99%, at least 99.5 or greater, identical to the nucleotide sequence (e.g., a fragment of a nucleotide sequence) shown in SEQ ID NO: 15 and to the nucleotide sequence (e.g., a fragment of a nucleotide sequence) shown in SEQ ID NO:11. In one embodiment, the CLIP1-ROS1 fusion comprises a nucleotide sequence containing at least 25, 50, 100, 150, 200, 500, 1000, 1500, 2000, 2500, 3000, or more nucleotides of the nucleotide sequence shown in SEQ ID NO:15 and a nucleotide sequence containing at least 25, 50, 100, 150, 200, 500, 1000, 1500, 2000, 2500, 3000, or more nucleotides of the nucleotide sequence shown in SEQ ID NO:11. In one embodiment, the CLIP1-ROS1 fusion comprises a nucleotide sequence containing at least 25, 50, 100, 150, 200, 500, 1000, 1500, 2000, 2500, 3000, or more contiguous nucleotides of the nucleotide sequence shown in SEQ ID NO: 15 and a nucleotide sequence containing at least 25, 50, 100, 150, 200, 500, 1000, 1500, 2000, 2500, 3000, or more nucleotides of the nucleotide sequence shown in SEQ ID NO: 11.

In another embodiment, the nucleic acid molecule includes a fusion, e.g., an in-frame fusion, of at least 1, 2, 3, 4, 5, 6, 7, 8, 9, 10, 11, 12, 13, 14, 15, 16, 17, 18, 19, 20 or more exons of CLIP1 or a fragment thereof (e.g., one or more of exons 1-20 of CLIP1 or a fragment thereof), and at least 1, 2, 3, 4, 5, 6, 7, 8 or more exons of ROS1 or a fragment thereof (e.g., one or more of exons 36-43 of ROS1 or a fragment thereof). In yet other embodiments, the nucleic acid molecule includes a fragment the nucleotide sequence shown in SEQ ID NO: 15 and a fragment of the nucleotide sequence shown in SEQ ID NO:11 or a fragment of the fusion, or a sequence substantially identical thereto.

In one embodiment, the nucleic acid molecule is complementary to at least a portion of a nucleotide sequence disclosed herein, e.g., is capable of hybridizing under a stringency condition described herein to SEQ ID NO: 15 and/or SEQ ID NO: 11, or a fragment thereof. In yet another embodiment, the nucleic acid molecule hybridizes to a nucleotide sequence that is complementary to at least a portion of a nucleotide sequence disclosed herein, e.g., is capable of hybridizing under a stringency condition to a nucleotide sequence complementary to SEQ ID NO: 15 and/or SEQ ID NO: 11, or a fragment thereof. The nucleotide sequence of a cDNA encoding an exemplary 5′ CLIP1-3′ ROS1 fusion is shown in at least exon 20 (e.g., exons 1-20) of SEQ ID NO:15 and at least exon 36 (e.g., exons 36-43) of SEQ ID NO: 11, and the predicted amino acid sequence is shown in the corresponding encoded exons of SEQ ID NO: 16 and the corresponding encoded exons of SEQ ID NO: 12, respectively.

In an embodiment the CLIP1-ROS1 nucleic acid molecule comprises sufficient CLIP1 and sufficient ROS1 sequence such that the encoded 5′ CLIP1-3′ ROS1 fusion has kinase activity, e.g., the fusion causes activation in the cells harboring it of oncogenic signaling pathways. In certain embodiments, the 5′ CLIP1-3′ ROS1 fusion comprises exons 1-20 from CLIP1 and exons 36-43 from ROS1. In certain embodiments, the CLIP1-ROS1 fusion comprises at least 1, 2, 3, 4, 5, 6, 7, 8, 9, 10, 11, 12, 13, 14, 15, 16, 17, 18, 19, 20 or more exons from CLIP1 and at least at least 1, 2, 3, 4, 5, 6, 7, 8 or more exons of ROS1. In certain embodiments, the CLIP1-ROS1 fusion comprises a fusion of exon 20 from CLIP1 and exon 36 from ROS1. In another embodiment, the CLIP1-ROS1 fusion comprises 1, 2, 3, 4, 5, 6, 7, 8, 9, 10, 11, 12, 13, 14, 15, 16, 17, 18, 19, 20 or more exons of CLIP1; and at least 1, 2, 3, 4, 5, 6, 7, 8 or more exons of ROS1.

In one embodiment, the nucleic acid molecule includes a nucleotide sequence that has an in-frame fusion of intron 20 of CLIP1 (e.g., NM_{—}002956) with intron 35 of ROS1 (e.g., NM_{—}002944). In another embodiment, the nucleic acid molecule includes a nucleotide sequence that includes a breakpoint. For example, the nucleic acid molecule includes a nucleotide sequence that includes the fusion junction between the CLIP1 gene and the ROS1 gene, e.g., the breakpoint between intron 20 of CLIP1 and intron 35 of ROS1. In other embodiments, the nucleic acid molecules includes a nucleotide sequence of one or more of nucleotide of chromosome 12 coupled to (e.g., directly or indirectly juxtaposed to) one or more of nucleotide of chromosome 6. In one embodiment, the nucleic acid molecule includes the nucleotide sequence of: chromosome 12 at one or more of a nucleotide plus or minus 10, 20, 30, 40 50, 60, 80, 100, 150 nucleotides and chromosome 6 at one or more of a nucleotide plus or minus 10, 20, 30, 40 50, 60, 80, 100, 150 nucleotides (corresponding to the breakpoint of a CLIP1-ROS1 fusion), or a fragment thereof, or a sequence substantially identical thereto. In one embodiment, the nucleic acid molecule is complementary to at least a portion of a nucleotide sequence disclosed herein, e.g., is capable of hybridizing under a stringency condition described herein to SEQ ID NO:15 and/or SEQ ID NO:11 or a fragment thereof. In yet other embodiment, the nucleic acid molecule hybridizes to a nucleotide sequence that is complementary to at least a portion of a nucleotide sequence disclosed herein, e.g., is capable of hybridizing under a stringency condition described herein to a nucleotide sequence complementary to SEQ ID NO:15 or SEQ ID NO:11 or a fragment thereof.

In another embodiment, the CLIP1-ROS1 fusion nucleic acid comprises at least 6, 12, 15, 20, 25, 50, 75, 100 or more nucleotides from exon 20 of CLIP1 (e.g., from the nucleotide sequence of CLIP1 preceding the fusion junction with ROS1, e.g., of the CLIP1 sequence shown in SEQ ID NO:15), and at least 6, 12, 15, 20, 25, 50, 75, 100 or more nucleotides from exon 36 of ROS1 (e.g., from the nucleotide sequence of ROS1 following the fusion junction with CLIP1, e.g., of the ROS1 sequence shown in SEQ ID NO: 11).

In other embodiments, the nucleic acid molecule includes a nucleotide sequence encoding a CLIP1-ROS1 fusion polypeptide that includes a fragment of a CLIP1 gene and a fragment of a ROS1 gene. In one embodiment, the nucleotide sequence encodes a CLIP1-ROS1 fusion polypeptide that includes e.g., a tyrosine kinase domain or a functional fragment thereof. In yet other embodiments, the nucleic acid molecule includes a nucleotide sequence encoding the amino acid sequence (e.g., a fragment of the amino acid sequence) shown in SEQ ID NO:16 and a nucleotide sequence encoding the amino acid sequence (e.g., a fragment of the amino acid sequence) shown in SEQ ID NO:12, or a fragment of the fusion, or a sequence substantially identical thereto. In one embodiment, the encoded CLIP1-ROS1 fusion polypeptide includes a tyrosine kinase domain or a functional fragment thereof.

In a related aspect, the invention features nucleic acid constructs that include the CLIP1-ROS1 nucleic acid molecules described herein. In certain embodiments, the nucleic acid molecules are operatively linked to a native or a heterologous regulatory sequence. Also included are vectors and host cells that include the CLIP1-ROS1 nucleic acid molecules described herein, e.g., vectors and host cells suitable for producing the nucleic acid molecules and polypeptides described herein.

In a related aspect, methods of producing the nucleic acid molecules and polypeptides described herein are also described.

In another aspect, the invention features nucleic acid molecules that reduce or inhibit the expression of a nucleic acid molecule that encodes a CLIP1-ROS1 fusion described herein. Examples of such nucleic acid molecules include, for example, antisense molecules, ribozymes, RNAi, triple helix molecules that hybridize to a nucleic acid encoding CLIP1-ROS1, or a transcription regulatory region of CLIP1-ROS1, and blocks or reduces mRNA expression of CLIP1-ROS1.

### Nucleic Acid Detection and Capturing Reagents

The invention also features a nucleic acid molecule, e.g., nucleic acid fragment, suitable as probe, primer, bait or library member that includes, flanks, hybridizes to, which are useful for identifying, or are otherwise based on, the CLIP1-ROS1 fusions described herein. In certain embodiments, the probe, primer or bait molecule is an oligonucleotide that allows capture, detection or isolation of a CLIP1-ROS1 fusion nucleic acid molecule described herein. The oligonucleotide can comprise a nucleotide sequence substantially complementary to a fragment of the CLIP1-ROS1 fusion nucleic acid molecules described herein. The sequence identity between the nucleic acid fragment, e.g., the oligonucleotide, and the target CLIP1-ROS1 sequence need not be exact, so long as the sequences are sufficiently complementary to allow the capture, detection or isolation of the target sequence. In one embodiment, the nucleic acid fragment is a probe or primer that includes an oligonucleotide between about 5 and 25, e.g., between 10 and 20, or 10 and 15 nucleotides in length. In other embodiments, the nucleic acid fragment is a bait that includes an oligonucleotide between about 100 to 300 nucleotides, 130 and 230 nucleotides, or 150 and 200 nucleotides, in length.

In one embodiment, the nucleic acid fragment can be used to identify or capture, e.g., by hybridization, a CLIP1-ROS1 fusion. For example, the nucleic acid fragment can be a probe, a primer, or a bait, for use in identifying or capturing, e.g., by hybridization, a CLIP1-ROS1 fusion described herein. In one embodiment, the nucleic acid fragment can be useful for identifying or capturing a CLIP1-ROS1 breakpoint, e.g., the nucleotide sequence of: chromosome 12 at nucleotide plus or minus 10, 20, 30, 40, 50, 60, 80, 100, 150 nucleotides and chromosome 6 at nucleotide plus or minus 10, 20, 30, 40 50, 60, 80, 100, 150 nucleotides.

In one embodiment, the nucleic acid fragment hybridizes to a nucleotide sequence within a chromosomal rearrangement that creates an in-frame fusion of intron 20 of CLIP1 with intron 35 of ROS1. In one embodiment, the nucleic acid fragment hybridizes to a nucleotide sequence in the region In other embodiments, the nucleic acid molecules includes a nucleotide sequence in the region of a nucleotide sequence of chromosome 12 coupled to (e.g., juxtaposed to) nucleotides in the region of a nucleotide sequence of chromosome 6. In one embodiment, the nucleic acid fragment hybridizes to a nucleotide sequence that includes a breakpoint, e.g., the nucleotide sequence of: chromosome 12 at nucleotide plus or minus 10, 20, 30, 40, 50, 60, 80, 100, 150 or more nucleotides and chromosome 6 at nucleotide plus or minus 10, 20, 30, 40, 50, 60, 80, 100, 150 or more nucleotides. For example, the nucleic acid fragment can hybridize to a nucleotide sequence that includes the fusion junction between the CLIP1 gene and the ROS1 gene, e.g., a nucleotide sequence that includes a portion of a nucleotide sequence within intron 20 of a CLIP1 gene and intron 35 of a ROS1 gene.

In another embodiment, the nucleic acid fragment hybridizes to a nucleotide sequence that comprises at least 6, 12, 15, 20, 25, 50, 75, 100, 150 or more nucleotides from exon 20 of CLIP1 (e.g., from the nucleotide sequence of CLIP1 preceding the fusion junction with ROS1, e.g., of the CLIP1 sequence shown in SEQ ID NO: 15), and at least 6, 12, 15, 20, 25, 50, 75, 100, 150 or more nucleotides from exon 36 of ROS1 (e.g., from the nucleotide sequence of ROS1 following the fusion junction with CLIP1, e.g., of the ROS1 sequence shown in SEQ ID NO: 11).

The probes or primers described herein can be used, for example, for FISH detection or PCR amplification. In one exemplary embodiment where detection is based on PCR, amplification of the CLIP1-ROS1 fusion junction fusion junction can be performed using a primer or a primer pair, e.g., for amplifying a sequence flanking the fusion junctions described herein, e.g., the mutations or the junction of a chromosomal rearrangement described herein, e.g., CLIP1-ROS1.

In one embodiment, a pair of isolated oligonucleotide primers can amplify a region containing or adjacent to a position in the CLIP1-ROS1 fusion. For example, forward primers can be designed to hybridize to a nucleotide sequence within CLIP1 genomic or mRNA sequence (e.g., a nucleotide sequence within exon 20 of CLIP1 of SEQ ID NO: 15), and the reverse primers can be designed to hybridize to a nucleotide sequence of ROS1 (e.g., a nucleotide sequence within exon 36 of ROS1, of SEQ ID NO:11).

In another embodiment, the nucleic acid fragments can be used to identify, e.g., by hybridization, a CLIP1-ROS1 fusion. In one embodiment, the nucleic acid fragment hybridizes to a nucleotide sequence that includes a fusion junction between the CLIP transcript and the ROS1 transcript.

In other embodiments, the nucleic acid fragment includes a bait that comprises a nucleotide sequence that hybridizes to a CLIP1-ROS1 fusion nucleic acid molecule described herein, and thereby allows the capture or isolation said nucleic acid molecule. In one embodiment, a bait is suitable for solution phase hybridization. In other embodiments, a bait includes a binding entity, e.g., an affinity tag, that allows capture and separation, e.g., by binding to a binding entity, of a hybrid formed by a bait and a nucleic acid hybridized to the bait.

In other embodiments, the nucleic acid fragment includes a library member comprising a CLIP1-ROS1 nucleic acid molecule described herein. In one embodiment, the library member includes a rearrangement that results in a CLIP1-ROS1 fusion described herein.

The nucleic acid fragment can be detectably labeled with, e.g., a radiolabel, a fluorescent label, a bioluminescent label, a chemiluminescent label, an enzyme label, a binding pair label, or can include an affinity tag; a tag, or identifier (e.g., an adaptor, barcode or other sequence identifier).

### CLIP1-ROS1 Fusion Polypeptides

In another embodiment, the CLIP1-ROS1 fusion comprises an amino acid sequence (e.g., a fragment of the amino acid sequence) shown in SEQ ID NO:16 and an amino acid sequence (e.g., a fragment of the amino acid sequence) shown in SEQ ID NO: 12, or a fragment of the fusion. In one embodiment, the CLIP1-ROS1 fusion comprises an amino acid sequence substantially identical to the amino acid sequence (e.g., a fragment of the amino acid sequence) shown in SEQ ID NO:16 and the amino acid sequence (e.g., a fragment of the amino acid sequence) shown in SEQ ID NO:12, or a fragment thereof. In one embodiment, the CLIP1-ROS1 fusion comprises an amino acid sequence at least 70%, at least 75%, at least 80%, at least 85%, at least 90%, at least 95%, at least 99%, at least 99.5 or greater, identical to the amino acid sequence (e.g., a fragment of the amino acid sequence) shown in SEQ ID NO: 16 and the amino acid sequence (e.g., a fragment of the amino acid sequence) shown in SEQ ID NO:12. In one embodiment, the CLIP1-ROS1 fusion comprises a sequence containing at least 10, 20, 50, 100, 500, 600, 700, 800, 900, 1000, or more amino acids of the amino acid sequence shown in SEQ ID NO:16 and SEQ ID NO:12. In one embodiment, the CLIP1-ROS1 fusion comprises an amino acid sequence containing at least 5, 10, 20, 50, 100, 500, 600, 700, 800, 900, 1000, or more contiguous amino acids of the amino acid sequence shown in SEQ ID NO:16 and at least 5, 10, 20, 50, 100, 500, 600, 700, 800, 900, 1000, or more contiguous amino acids of the amino acid sequence shown in SEQ ID NO:12. In one embodiment, the 5′ CLIP1-3′ ROS1 fusion polypeptide includes a receptor tyrosine kinase domain or a functional fragment thereof. In an embodiment, the 5′CLIP1-3′ROS1 fusion polypeptide comprises sufficient ROS1 and sufficient CLIP1 sequence such that it has kinase activity, e.g., has elevated activity.

In another aspect, the invention features a CLIP1-ROS1 fusion polypeptide (e.g., a purified CLIP1-ROS1 fusion polypeptide), a biologically active or antigenic fragment thereof, as well as reagents (e.g., antibody molecules that bind to a CLIP1-ROS1 fusion polypeptide), methods for modulating a CLIP1-ROS1 polypeptide activity and detection of a CLIP1-ROS1 polypeptide.

In one embodiment, the CLIP1-ROS1 fusion polypeptide has at least one biological activity. In one embodiment, at least one biological activity of the CLIP1-ROS1 fusion polypeptide is reduced or inhibited by an anti-cancer drug, e.g., a CLIP1 inhibitor, a ROS1 inhibitor. In one embodiment, at least one biological activity of the CLIP1-ROS1 fusion polypeptide is reduced or inhibited by a ROS1 inhibitor. In one embodiment, at least one biological activity of the CLIP1-ROS1 fusion polypeptide is reduced or inhibited by a CLIP1 inhibitor. In one embodiment, at least one biological activity of the CLIP1-ROS1 fusion polypeptide is reduced or inhibited by a ROS1 inhibitor, e.g., Ganetespib; Crizotinib; TAE684; AP26113, X-276, X-376, X-396, CH5424802 (AF-802), GSK1838705, ASP3026, PHA-E429, CRL151104A; and additional examples of kinase inhibitors are described in de la Bellacasa R. P. et al, Transl Lung Cancer Res 2013; 2(2):72-86.

In yet other embodiments, the CLIP1-ROS1 fusion polypeptide is encoded by a nucleic acid molecule described herein. In one embodiment, the CLIP1-ROS1 fusion polypeptide is encoded by an in-frame fusion of intron 20 of CLIP1 with intron 35 of ROS1 (e.g., a sequence on chromosome 12 and a sequence on chromosome 6). In another embodiment, the CLIP1-ROS1 fusion polypeptide includes an amino acid sequence encoded by a nucleotide sequence comprising a fusion junction between the CLIP1 transcript and the ROS1 transcript.

In certain embodiments, the CLIP1-ROS1 fusion polypeptide comprises one or more of encoded exons 1-20 from CLIP1 and one or more of encoded exons 36-43 of ROS1. In certain embodiments, the CLIP1-ROS1 fusion polypeptide comprises at least 1, 2, 3, 4, 5, 6, 7, 8, 9, 10, 11, 12, 13, 14, 15, 16, 17, 18, 19, 20 or more encoded exons of CLIP1 and at least at least 1, 2, 3, 4, 5, 6, 7, 8 or more encoded exons of ROS1. In certain embodiments, the CLIP1-ROS1 fusion polypeptide comprises a fusion of encoded exon 20 from CLIP1 and encoded exon 36 from ROS1 (or a fragment thereof). In other embodiments, the fusion comprises at least 1, 2, 3, 4, 5, 6, 7, 8, 9, 10, 11, 12, 13, 14, 15, 16, 17, 18, 19, 20 or more encoded exons of CLIP1; and at least 1, 2, 3, 4, 5, 6, 7, 8 or more encoded exons of ROS1. In certain embodiments, the CLIP1-ROS1 fusion polypeptide comprises encoded exons 1-20 from CLIP1 and exons 36-43 of ROS1. In certain embodiments, the 5′ CLIP1-3′ ROS1 fusion polypeptide comprises a fusion junction of the sequence of exon 20 from CLIP1 and the sequence of exon 36 from ROS1.

In certain embodiments, the CLIP1-ROS1 fusion comprises the amino acid sequence corresponding to exon 20 or a fragment thereof from CLIP1, and the amino acid sequence corresponding to exon 36 or a fragment thereof from ROS1 (e.g., as shown in SEQ ID NO: 16 and SEQ ID NO:12). In one embodiment, the CLIP1-ROS1 fusion comprises at least 5, 10, 15, 20 or more amino acids from exon 20 of CLIP1 (e.g., from the amino acid sequence of CLIP1 preceding the fusion junction with ROS1, e.g., of the CLIP1 sequence shown in SEQ ID NO:16), and at least 5, 10, 15, 20 or more amino acids from exon 36 of ROS1 (e.g., from the amino acid sequence of ROS1 following the fusion junction with CLIP1, e.g., of the ROS1 sequence shown in SEQ ID NO: 12).

In one embodiment, the CLIP1-ROS1 fusion polypeptide includes a tyrosine kinase domain or a functional fragment thereof. In a related aspect, the invention features CLIP1-ROS1 fusion polypeptide or fragments operatively linked to heterologous polypeptides to form fusion proteins.

In another embodiment, the CLIP1-ROS1 fusion polypeptide or fragment is a peptide, e.g., an immunogenic peptide or protein. This peptide or protein contains a fusion junction described herein. Such immunogenic peptides or proteins can be used to raise antibodies specific to the fusion protein. In other embodiments, such immunogenic peptides or proteins can be used for vaccine preparation. The vaccine preparation can include other components, e.g., an adjuvant.

In another aspect, the invention features antibody molecules that bind to a CLIP1-ROS1 fusion polypeptide or fragment described herein. In embodiments, the antibody can distinguish wild type ROS1 (or CLIP1) from CLIP1-ROS1.

### Detection Reagents and Detection of Mutations

In another aspect, the invention features a detection reagent, e.g., a purified or an isolated preparation thereof. Detection reagents can distinguish a nucleic acid, or protein sequence, having a breakpoint, e.g., a CLIP1-ROS1 breakpoint; from a reference sequence. In one embodiment, the detection reagent detects (e.g., specifically detects) a CLIP1-ROS1 fusion nucleic acid or a polypeptide, e.g., distinguishes a wild type ROS1 or another ROS1 fusion (or CLIP1) from a CLIP1-ROS1 nucleic acid (e.g., as described herein in SEQ ID NO: 15 and SEQ ID NO: 11); or a CLIP1-ROS1 polypeptide (e.g., as described herein in SEQ ID NO: 16 and SEQ ID NO: 12).

Detection reagents, e.g., nucleic acid-based detection reagents, can be used to identify mutations in a target nucleic acid. e.g., DNA, e.g., genomic DNA or cDNA, or RNA, e.g., in a sample, e.g., a sample of nucleic acid derived from a neoplastic or tumor cell, e.g., a melanocytic neoplasm, melanoma or metastatic cell. Detection reagents, e.g., antibody-based detection reagents, can be used to identify mutations in a target protein, e.g., in a sample, e.g., a sample of protein derived from, or produced by, a neoplastic or tumor cell, e.g., a melanocytic neoplasm, melanoma or metastatic cell.

### Method of Treatment

In a related aspect, a method of inhibiting, reducing, or treating a hyperproliferative disorder, e.g., a neoplasm (including benign, pre-malignant or malignant (e.g., a cancer), in a subject is provided. The method includes administering to the subject a preselected therapeutic agent, e.g., an anti-cancer agent (e.g., a kinase inhibitor), as a single agent, or in combination, in an amount sufficient to reduce, inhibit or treat the activity or expression of CLIP1-ROS1 (e.g., a CLIP1-ROS1 fusion described herein), thereby inhibiting, reducing, or treating the hyperproliferative disorder in the subject.

In one embodiment, the subject treated has a CLIP1-ROS1 fusion; e.g., the subject has a tumor or cancer harboring a CLIP1-ROS1 fusion. In other embodiments, the subject has been previously identified as having a CLIP1-ROS1 fusion. In yet other embodiments, the subject has been previously identified as being likely or unlikely to respond to treatment with a protein kinase inhibitor, e.g., a subject that has previously participated in a clinical trial. In other embodiments, the subject has been previously identified as being likely or unlikely to respond to treatment with a protein kinase inhibitor, based on the presence of the CLIP1-ROS1 fusion. In one embodiment, the subject is a mammal, e.g., a human. In one embodiment, the subject has, or at risk of having a cancer at any stage of disease. In other embodiments, the subject is a patient, e.g., a cancer patient.

In certain embodiments, the neoplasm or neoplastic cell is a benign, pre-malignant, malignant (cancer) or metastasis. In certain embodiments, the cancer is a solid tumor, a soft tissue tumor, or a metastatic lesion.

In one embodiment, the neoplasm or neoplastic cell is a benign, pre-malignant, malignant (cancer) or metastasis. In certain embodiments, the cancer is a solid tumor, a soft tissue tumor, or a metastatic lesion. In one embodiment, the cancer is a carcinoma. In one embodiment, the cancer is chosen from a lung cancer, a pancreatic cancer, melanoma, a colorectal cancer, an esophageal-gastric cancer, a thyroid cancer, or an adenocarcinoma. In one embodiment, the cancer is a lung adenocarcinoma. In other embodiment, the lung cancer is chosen from one or more of the following: non-small cell lung cancer (NSCLC), small cell lung cancer (SCLC), squamous cell carcinoma (SCC), adenocarcinoma of the lung, bronchogenic carcinoma, or a combination thereof. In one embodiment, the lung cancer is NSCLC or SCC.

In one embodiment, the anti-cancer agent is a ROS1 inhibitor. In one embodiment, the anti-cancer agent is a CLIP1 inhibitor. In one embodiment, the anti-cancer agent is a kinase inhibitor. In one embodiment, the anti-cancer agent is a ROS1 inhibitor, e.g., Ganetespib; Crizotinib; TAE684; AP26113, X-276, X-376, X-396, CH5424802 (AF-802), GSK1838705, ASP3026, PHA-E429, CRL151104; and additional examples of kinase inhibitors are described in de la Bellacasa R. P. et al, Transl Lung Cancer Res 2013; 2(2):72-86.

### PPFIBP1-ROS1 Fusions

In one embodiment, a fusion includes an in-frame fusion of an exon of PTPRF interacting protein binding protein 1 (liprin beta 1) (PPFIBP1), e.g., one more exons of PPFIBP1 (e.g., one or more of exons 1-9 of PPFIBP1) or a fragment thereof, and an exon of C-Ros oncogene 1 (ROS1), e.g., one or more exons of a ROS1 (e.g., one or more of exons 35-43 of ROS1) or a fragment thereof. For example, the PPFIBP1-ROS1 fusion can include an in-frame fusion within an intron of PPFIBP1 (e.g., intron 9) or a fragment thereof, with an intron of ROS1 (e.g., intron 34) or a fragment thereof. In one embodiment, the fusion of the PPFIBP1-ROS1 fusion comprises the nucleotide sequence of: chromosome 12 at one or more of a nucleotide (plus or minus 10, 20, 30, 50, 60, 70, 80, 100 or more nucleotides) and chromosome 6 at one or more of a nucleotide (plus or minus 10, 20, 30, 50, 60, 70, 80, 100 or more nucleotides). In one embodiment, the PPFIBP1-ROS1 fusion is a translocation, e.g., a translocation of a portion of chromosome 12 and a portion of chromosome 6.

In certain embodiments, the PPFIBP1-ROS1 fusion is in a 5′-PPFIBP1 to 3′-ROS1 configuration (also referred to herein as “5′-PPFIBP1-ROS1-3′).” The term “fusion” or “fusion molecule” can refer to a polypeptide or a nucleic acid fusion, depending on the context. It may include a full-length sequence of a fusion or a fragment thereof, e.g., a fusion junction (e.g., a fragment including a portion of PPFIBP1 and a portion of ROS1, e.g., a portion of the PPFIBP1-ROS1 fusion described herein). In one embodiment, the PPFIBP1-ROS1 fusion polypeptide includes a fragment of the amino acid sequence shown in SEQ ID NO:20 and a fragment of the amino acid sequence shown in SEQ ID NO:12, or an amino acid sequence substantially identical thereto. In another embodiment, the PPFIBP1-ROS1 fusion nucleic acid includes a fragment of the nucleotide sequence shown in SEQ ID NO: 19 and a fragment of the nucleotide sequence shown in SEQ ID NO:11, or a nucleotide sequence substantially identical thereto. In one embodiment, the PPFIBP1-ROS1 fusion polypeptide comprises sufficient PPFIBP1 and sufficient ROS1 sequence such that the 5′ PPFIBP1-3′ ROS1 fusion has kinase activity, e.g., has elevated activity, e.g., tyrosine kinase activity and in any event, it causes activation in the cells harboring this fusion of oncogenic signalling pathways.

In certain embodiments, the PPFIBP1-ROS1 fusion comprises one or more (or all of) exons 1-9 from PPFIBP1 and one or more (or all of) exons 35-43 of ROS1 (e.g., one or more of the exons shown in SEQ ID NO:19 and SEQ ID NO: 11. In another embodiment, the PPFIBP1-ROS1 fusion comprises one or more (or all of) exons 1-9 of PPFIBP1 and one or more (or all of) exons 35-43 of ROS1. In certain embodiments, the PPFIBP1-ROS1 fusion comprises at least 1, 2, 3, 4, 5, 6, 7, 8, 9 or more exons (or encoded exons) from PPFIBP1 and at least 1, 2, 3, 4, 5, 6, 7, 8, 9 or more exons (or encoded exons) from ROS1 (e.g., from the PPFIBP1 and ROS1 sequences shown in SEQ ID NO: 19 and SEQ ID NO:20 and SEQ ID NO:11 and SEQ ID NO:12.

In certain embodiments, the PPFIBP1-ROS1 fusion comprises exons 1-9 or a fragment thereof from PPFIBP1, and exons 35-43 or a fragment thereof from ROS1 (e.g., as shown in SEQ ID NO:19 and SEQ ID NO: 11). In one embodiment, the PPFIBP1-ROS1 fusion comprises at least 5, 10, 15, 20, 30, 40, 50 or more amino acids from exons 1-9 of PPFIBP1 (e.g., from the amino acid sequence of PPFIBP1 as shown in SEQ ID NO:20 (e.g., from the amino acid sequence of PPFIBP1 preceding the fusion junction with ROS1, and at least 5, 10, 15, 20, 30, 40, 50 or more amino acids from exons 35-43 of ROS1 (e.g., from the amino acid sequence of ROS1 as shown in SEQ ID NO: 12). In another embodiment, the PPFIBP1-ROS1 fusion comprises at least 6, 12, 15, 20, 25, 50, 75, 100 or more nucleotides from exons 1-9 of PPFIBP1 (e.g., from the nucleotide sequence of PPFIBP1 as shown in SEQ ID NO: 19 (e.g., from the nucleotide sequence of PPFIBP1 preceding the fusion junction with ROS1); and at least 6, 12, 15, 20, 25, 50, 75, 100 or more nucleotides from exons 35-43 of ROS1 (e.g., from the nucleotide sequence of ROS1 as shown in SEQ ID NO: 11).

### PPFIBP1-ROS1 Nucleic Acid Molecules

In one aspect, the invention features a nucleic acid molecule (e.g., an isolated or purified) nucleic acid molecule that includes a fragment of a PPFIBP1 gene and a fragment of a ROS1 gene. In one embodiment, the nucleotide sequence encodes a PPFIBP1-ROS1 fusion polypeptide that includes a tyrosine kinase domain or a functional fragment thereof. In another embodiment, the nucleotide sequence encodes a fragment of the ROS1 polypeptide including the amino acid sequence of SEQ ID NO: 12 or a fragment thereof, or a sequence substantially identical thereto. In other embodiments, the nucleic acid molecule includes a fragment of the PPFIBP1 gene encoding the amino acid sequence of SEQ ID NO:20 or a fragment thereof, or a sequence substantially identical thereto. In yet other embodiments, the nucleic acid molecule includes a nucleotide sequence encoding the amino acid sequence shown in SEQ ID NO:20, or a fragment thereof, and the amino acid sequence shown in SEQ ID NO: 12 or a fragment thereof, or a sequence substantially identical thereto.

In one embodiment, the nucleic acid molecule includes a fusion, e.g., an in-frame fusion, between an intron of PPFIBP1 (e.g., intron 9, or a fragment thereof), and an intron of ROS1 (e.g., intron 34, or a fragment thereof). The PPFIBP1-ROS1 fusion can comprise a fusion of the nucleotide sequence of: chromosome 12 at one or more of a nucleotide (plus or minus 10, 20, 30, 50, 60, 70, 80, 100 nucleotides) and chromosome 6 at one or more of a nucleotide (plus or minus 10, 20, 30, 50, 60, 70, 80, 100 nucleotides), or a fragment thereof. In one embodiment, the PPFIBP1-ROS1 fusion comprises a fusion of the nucleotide sequence of: chromosome 12 at one or more of a nucleotide (plus or minus 10, 20, 30, 50, 60, 70, 80, 100 nucleotides) and chromosome 6 at one or more of a nucleotide (plus or minus 10, 20, 30, 50, 60, 70, 80, 100 nucleotides), or a fragment thereof.

In another embodiment, the PPFIBP1-ROS1 fusion comprises a nucleotide sequence (e.g., a fragment of a nucleotide sequence) shown in SEQ ID NO:19 and a nucleotide sequence (e.g., a fragment of a nucleotide sequence) shown in SEQ ID NO: 11, or a fragment of the fusion. In one embodiment, the PPFIBP1-ROS1 fusion comprises a nucleotide sequence substantially identical to the nucleotide sequence (e.g., a fragment of a nucleotide sequence) shown in SEQ ID NO: 19 and the nucleotide sequence (e.g., a fragment of a nucleotide sequence) shown SEQ ID NO: 11, or a fragment of the fusion. In one embodiment, the PPFIBP1-ROS1 fusion comprises a nucleotide sequence at least 70%, at least 75%, at least 80%, at least 85%, at least 90%, at least 95%, at least 99%, at least 99.5 or greater, identical to the nucleotide sequence (e.g., a fragment of a nucleotide sequence) shown in SEQ ID NO: 19 and to the nucleotide sequence (e.g., a fragment of a nucleotide sequence) shown in SEQ ID NO: 11. In one embodiment, the PPFIBP1-ROS1 fusion comprises a nucleotide sequence containing at least 25, 50, 100, 150, 200, 500, 1000, 1500, 2000, 2500, 3000, or more nucleotides of the nucleotide sequence shown in SEQ ID NO: 19 and a nucleotide sequence containing at least 25, 50, 100, 150, 200, 500, 1000, 1500, 2000, 2500, 3000, or more nucleotides of the nucleotide sequence shown in SEQ ID NO:11. In one embodiment, the PPFIBP1-ROS1 fusion comprises a nucleotide sequence containing at least 25, 50, 100, 150, 200, 500, 1000, 1500, 2000, 2500, 3000, or more contiguous nucleotides of the nucleotide sequence shown in SEQ ID NO: 19 and a nucleotide sequence containing at least 25, 50, 100, 150, 200, 500, 1000, 1500, 2000, 2500, 3000, or more nucleotides of the nucleotide sequence shown in SEQ ID NO:11.

In another embodiment, the nucleic acid molecule includes a fusion, e.g., an in-frame fusion, of at least 1, 2, 3, 4, 5, 6, 7, 8, 9 or more exons of PPFIBP1 or a fragment thereof (e.g., one or more of exons 1-9 of PPFIBP1 or a fragment thereof), and at least 1, 2, 3, 4, 5, 6, 7, 8, 9 or more exons of ROS1 or a fragment thereof (e.g., one or more of exons 35-43 of ROS1 or a fragment thereof). In yet other embodiments, the nucleic acid molecule includes a fragment the nucleotide sequence shown in SEQ ID NO:19 and a fragment of the nucleotide sequence shown in SEQ ID NO:11 or a fragment of the fusion, or a sequence substantially identical thereto.

In one embodiment, the nucleic acid molecule is complementary to at least a portion of a nucleotide sequence disclosed herein, e.g., is capable of hybridizing under a stringency condition described herein to SEQ ID NO: 19 and/or SEQ ID NO: 11, or a fragment thereof. In yet another embodiment, the nucleic acid molecule hybridizes to a nucleotide sequence that is complementary to at least a portion of a nucleotide sequence disclosed herein, e.g., is capable of hybridizing under a stringency condition to a nucleotide sequence complementary to SEQ ID NO: 19 and/or SEQ ID NO: 11, or a fragment thereof. The nucleotide sequence of a cDNA encoding an exemplary 5′ PPFIBP1-3′ ROS1 fusion is shown in at least exon 9 (e.g., exons 1-9) of SEQ ID NO:19 and at least exon 35 (e.g., exons 35-43) of SEQ ID NO: 11, and the predicted amino acid sequence is shown in the corresponding encoded exons of SEQ ID NO:20 and the corresponding encoded exons of SEQ ID NO: 12, respectively.

In an embodiment the PPFIBP1-ROS1 nucleic acid molecule comprises sufficient PPFIBP1 and sufficient ROS1 sequence such that the encoded 5′ PPFIBP1-3′ ROS1 fusion has kinase activity, e.g., has elevated activity. In any event, the fusion causes activation in the cells harboring it of oncogenic signalling pathways. In certain embodiments, the 5′ PPFIBP1-3′ ROS1 fusion comprises exons 1-9 from PPFIBP1 and exons 35-43 from ROS1. In certain embodiments, the PPFIBP1-ROS1 fusion comprises at least 1, 2, 3, 4, 5, 6, 7, 8, 9 or more exons from PPFIBP1 and at least at least 1, 2, 3, 4, 5, 6, 7, 8, 9 or more exons of ROS1. In certain embodiments, the PPFIBP1-ROS1 fusion comprises a fusion of exon 9 from PPFIBP1 and exon 35 from ROS1. In another embodiment, the PPFIBP1-ROS1 fusion comprises 1, 2, 3, 4, 5, 6, 7, 8, 9 or more exons of PPFIBP1; and at least 1, 2, 3, 4, 5, 6, 7, 8, 9 or more exons of ROS1.

In one embodiment, the nucleic acid molecule includes a nucleotide sequence that has an in-frame fusion of intron 9 of PPFIBP1 (e.g., NM_{—}003622 with intron 34 of ROS1 (e.g., NM_{—}002944). In another embodiment, the nucleic acid molecule includes a nucleotide sequence that includes a breakpoint. For example, the nucleic acid molecule includes a nucleotide sequence that includes the fusion junction between the PPFIBP1 gene and the ROS1 gene, e.g., the breakpoint between intron 9 of PPFIBP1 and intron 34 of ROS1. In other embodiments, the nucleic acid molecules includes a nucleotide sequence of one or more of nucleotide of chromosome 12 coupled to (e.g., directly or indirectly juxtaposed to) one or more of nucleotide of chromosome 6. In one embodiment, the nucleic acid molecule includes the nucleotide sequence of: chromosome 12 at one or more of a nucleotide plus or minus 10, 20, 30, 40 50, 60, 80, 100, 150 nucleotides and chromosome 6 at one or more of a nucleotide plus or minus 10, 20, 30, 40, 50, 60, 80, 100, 150 nucleotides (corresponding to the breakpoint of a PPFIBP1-ROS1 fusion), or a fragment thereof, or a sequence substantially identical thereto. In one embodiment, the nucleic acid molecule is complementary to at least a portion of a nucleotide sequence disclosed herein, e.g., is capable of hybridizing under a stringency condition described herein to SEQ ID NO: 19 and/or SEQ ID NO: 11 or a fragment thereof. In yet other embodiment, the nucleic acid molecule hybridizes to a nucleotide sequence that is complementary to at least a portion of a nucleotide sequence disclosed herein, e.g., is capable of hybridizing under a stringency condition described herein to a nucleotide sequence complementary to SEQ ID NO: 19 or SEQ ID NO: 11 or a fragment thereof.

In another embodiment, the PPFIBP1-ROS1 fusion nucleic acid comprises at least 6, 12, 15, 20, 25, 50, 75, 100 or more nucleotides from exon 9 of PPFIBP1 (e.g., from the nucleotide sequence of PPFIBP1 preceding the fusion junction with ROS1, e.g., of the PPFIBP1 sequence shown in SEQ ID NO:19), and at least 6, 12, 15, 20, 25, 50, 75, 100 or more nucleotides from exon 35 of ROS1 (e.g., from the nucleotide sequence of ROS1 following the fusion junction with PPFIBP1, e.g., of the ROS sequence shown in SEQ ID NO: 12).

In other embodiments, the nucleic acid molecule includes a nucleotide sequence encoding a PPFIBP1-ROS1 fusion polypeptide that includes a fragment of a PPFIBP1 gene and a fragment of a ROS1 gene. In one embodiment, the nucleotide sequence encodes a PPFIBP1-ROS1 fusion polypeptide that includes e.g., a tyrosine kinase domain or a functional fragment thereof. In yet other embodiments, the nucleic acid molecule includes a nucleotide sequence encoding the amino acid sequence (e.g., a fragment of the amino acid sequence) shown in SEQ ID NO:20 and a nucleotide sequence encoding the amino acid sequence (e.g., a fragment of the amino acid sequence) shown in SEQ ID NO: 12, or a fragment of the fusion, or a sequence substantially identical thereto. In one embodiment, the encoded PPFIBP1-ROS1 fusion polypeptide includes a tyrosine kinase domain or a functional fragment thereof.

In a related aspect, the invention features nucleic acid constructs that include the PPFIBP1-ROS1 nucleic acid molecules described herein. In certain embodiments, the nucleic acid molecules are operatively linked to a native or a heterologous regulatory sequence. Also included are vectors and host cells that include the PPFIBP1-ROS1 nucleic acid molecules described herein, e.g., vectors and host cells suitable for producing the nucleic acid molecules and polypeptides described herein.

In a related aspect, methods of producing the nucleic acid molecules and polypeptides described herein are also described.

In another aspect, the invention features nucleic acid molecules that reduce or inhibit the expression of a nucleic acid molecule that encodes a PPFIBP1-ROS1 fusion described herein. Examples of such nucleic acid molecules include, for example, antisense molecules, ribozymes. RNAi, triple helix molecules that hybridize to a nucleic acid encoding PPFIBP1-ROS1, or a transcription regulatory region of PPFIBP1-ROS1, and blocks or reduces mRNA expression of PPFIBP1-ROS1.

In one embodiment, the PPFIBP1-ROS1 fusion includes the full sequence or a fragment of the nucleotide sequence shown in SEQ ID NO:17 and SEQ ID NO:86, or a nucleotide sequence substantially identical thereto, e.g., 70% identical or 80% or 90% or more identical. In another embodiment, the PPFIBP1-ROS1 fusion polypeptide includes the full sequence or a fragment of the amino acid sequence shown in SEQ ID NO: 18 and SEQ ID NO:87, or an amino acid sequence substantially identical thereto.

### Nucleic Acid Detection and Capturing Reagents

The invention also features a nucleic acid molecule, e.g., nucleic acid fragment, suitable as probe, primer, bait or library member that includes, flanks, hybridizes to, which are useful for identifying, or are otherwise based on, the PPFIBP1-ROS1 fusions described herein. In certain embodiments, the probe, primer or bait molecule is an oligonucleotide that allows capture, detection or isolation of a PPFIBP1-ROS1 fusion nucleic acid molecule described herein. The oligonucleotide can comprise a nucleotide sequence substantially complementary to a fragment of the PPFIBP1-ROS1 fusion nucleic acid molecules described herein. The sequence identity between the nucleic acid fragment, e.g., the oligonucleotide, and the target PPFIBP1-ROS1 sequence need not be exact, so long as the sequences are sufficiently complementary to allow the capture, detection or isolation of the target sequence. In one embodiment, the nucleic acid fragment is a probe or primer that includes an oligonucleotide between about 5 and 25, e.g., between 10 and 20, or 10 and 15 nucleotides in length. In other embodiments, the nucleic acid fragment is a bait that includes an oligonucleotide between about 100 to 300 nucleotides, 130 and 230 nucleotides, or 150 and 200 nucleotides, in length.

In one embodiment, the nucleic acid fragment can be used to identify or capture. e.g., by hybridization, a PPFIBP1-ROS1 fusion. For example, the nucleic acid fragment can be a probe, a primer, or a bait, for use in identifying or capturing, e.g., by hybridization, a PPFIBP1-ROS1 fusion described herein. In one embodiment, the nucleic acid fragment can be useful for identifying or capturing a PPFIBP1-ROS1 breakpoint, e.g., the nucleotide sequence of: chromosome 12 at nucleotide plus or minus 10, 20, 30, 40, 50, 60, 80, 100, 150 nucleotides and chromosome 6 at nucleotide plus or minus 10, 20, 30, 40 50, 60, 80, 100, 150 nucleotides.

In one embodiment, the nucleic acid fragment hybridizes to a nucleotide sequence within a chromosomal rearrangement that creates an in-frame fusion of intron 9 of PPFIBP1 with intron 34 of ROS1. In one embodiment, the nucleic acid fragment hybridizes to a nucleotide sequence in the region In other embodiments, the nucleic acid molecules includes a nucleotide sequence in the region of a nucleotide sequence of chromosome 12 coupled to (e.g., juxtaposed to) nucleotides in the region of a nucleotide sequence Y of chromosome 6. In one embodiment, the nucleic acid fragment hybridizes to a nucleotide sequence that includes a breakpoint, e.g., the nucleotide sequence of: chromosome 12 at nucleotide plus or minus 10, 20, 30, 40, 50, 60, 80, 100, 150 or more nucleotides and chromosome 6 at nucleotide plus or minus 10, 20, 30, 40, 50, 60, 80, 100, 150 or more nucleotides. For example, the nucleic acid fragment can hybridize to a nucleotide sequence that includes the fusion junction between the PPFIBP1 gene and the ROS1 gene, e.g., a nucleotide sequence that includes a portion of a nucleotide sequence within intron 9 of a PPFIBP1 gene and intron 34 of a ROS1 gene.

In another embodiment, the nucleic acid fragment hybridizes to a nucleotide sequence that comprises at least 6, 12, 15, 20, 25, 50, 75, 100, 150 or more nucleotides from exon 9 of PPFIBP1 (e.g., from the nucleotide sequence of PPFIBP1 preceding the fusion junction with ROS1, e.g., of the PPFIBP1 sequence shown in SEQ ID NO: 19), and at least 6, 12, 15, 20, 25, 50, 75, 100, 150 or more nucleotides from exon 35 of ROS1 (e.g., from the nucleotide sequence of ROS1 following the fusion junction with PPFIBP1, e.g., of the ROS1 sequence shown in SEQ ID NO: 11).

The probes or primers described herein can be used, for example, for FISH detection or PCR amplification. In one exemplary embodiment where detection is based on PCR, amplification of the PPFIBP1-ROS1 fusion junction fusion junction can be performed using a primer or a primer pair, e.g., for amplifying a sequence flanking the fusion junctions described herein, e.g., the mutations or the junction of a chromosomal rearrangement described herein, e.g., PPFIBP1-ROS1.

In one embodiment, a pair of isolated oligonucleotide primers can amplify a region containing or adjacent to a position in the PPFIBP1-ROS1 fusion. For example, forward primers can be designed to hybridize to a nucleotide sequence within PPFIBP1 genomic or mRNA sequence (e.g., a nucleotide sequence within exon 9 of PPFIBP1 of SEQ ID NO: 19), and the reverse primers can be designed to hybridize to a nucleotide sequence of ROS1 (e.g., a nucleotide sequence within exon 35 of ROS1, of SEQ ID NO: 1).

In another embodiment, the nucleic acid fragments can be used to identify, e.g., by hybridization, a PPFIBP1-ROS1 fusion. In one embodiment, the nucleic acid fragment hybridizes to a nucleotide sequence that includes a fusion junction between the PPFIBP1 transcript and the ROS1 transcript.

In other embodiments, the nucleic acid fragment includes a bait that comprises a nucleotide sequence that hybridizes to a PPFIBP1-ROS1 fusion nucleic acid molecule described herein, and thereby allows the capture or isolation said nucleic acid molecule. In one embodiment, a bait is suitable for solution phase hybridization. In other embodiments, a bait includes a binding entity. e.g., an affinity tag, that allows capture and separation, e.g., by binding to a binding entity, of a hybrid formed by a bait and a nucleic acid hybridized to the bait.

In other embodiments, the nucleic acid fragment includes a library member comprising a PPFIBP1-ROS1 nucleic acid molecule described herein. In one embodiment, the library member includes a rearrangement that results in a PPFIBP1-ROS1 fusion described herein.

The nucleic acid fragment can be detectably labeled with, e.g., a radiolabel, a fluorescent label, a bioluminescent label, a chemiluminescent label, an enzyme label, a binding pair label, or can include an affinity tag; a tag, or identifier (e.g., an adaptor, barcode or other sequence identifier).

### PPFIBP1-ROS1 Fusion Polypeptides

In another embodiment, the PPFIBP1-ROS1 fusion comprises an amino acid sequence (e.g., a fragment of the amino acid sequence) shown in SEQ ID NO:20 and an amino acid sequence (e.g., a fragment of the amino acid sequence) shown in SEQ ID NO: 12, or a fragment of the fusion. In one embodiment, the PPFIBP1-ROS1 fusion comprises an amino acid sequence substantially identical to the amino acid sequence (e.g., a fragment of the amino acid sequence) shown in SEQ ID NO:20 and the amino acid sequence (e.g., a fragment of the amino acid sequence) shown in SEQ ID NO:12, or a fragment thereof. In one embodiment, the PPFIBP1-ROS1 fusion comprises an amino acid sequence at least 70%, at least 75%, at least 80%, at least 85%, at least 90%, at least 95%, at least 99%, at least 99.5 or greater, identical to the amino acid sequence (e.g., a fragment of the amino acid sequence) shown in SEQ ID NO:20 and the amino acid sequence (e.g., a fragment of the amino acid sequence) shown in SEQ ID NO:12. In one embodiment, the PPFIBP1-ROS1 fusion comprises a sequence containing at least 10, 20, 50, 100, 500, 600, 700, 800, 900, 1000, or more amino acids of the amino acid sequence shown in SEQ ID NO:20 and SEQ ID NO: 12. In one embodiment, the PPFIBP1-ROS1 fusion comprises an amino acid sequence containing at least 5, 10, 20, 50, 100, 500, 600, 700, 800, 900, 1000, or more contiguous amino acids of the amino acid sequence shown in SEQ ID NO:20 and at least 5, 10, 20, 50, 100, 500, 600, 700, 800, 900, 1000, or more contiguous amino acids of the amino acid sequence shown in SEQ ID NO:12. In one embodiment, the 5′ PPFIBP1-3′ ROS1 fusion polypeptide includes a receptor tyrosine kinase domain or a functional fragment thereof. In an embodiment, the 5′PPFIBP1-3′ROS1 fusion polypeptide comprises sufficient ROS1 and sufficient PPFIBP1 sequence such that it has kinase activity, e.g., has elevated activity. In any event, the fusion causes activation in the cells harboring it of oncogenic signaling pathways.

In another aspect, the invention features a PPFIBP1-ROS1 fusion polypeptide (e.g., a purified PPFIBP1-ROS1 fusion polypeptide), a biologically active or antigenic fragment thereof, as well as reagents (e.g., antibody molecules that bind to a PPFIBP1-ROS1 fusion polypeptide), methods for modulating a PPFIBP1-ROS1 polypeptide activity and detection of a PPFIBP1-ROS1 polypeptide.

In one embodiment, the PPFIBP1-ROS1 fusion polypeptide has at least one biological activity. In one embodiment, at least one biological activity of the PPFIBP1-ROS1 fusion polypeptide is reduced or inhibited by an anti-cancer drug, e.g., a PPFIBP1 inhibitor, a ROS1 inhibitor. In one embodiment, at least one biological activity of the PPFIBP1-ROS1 fusion polypeptide is reduced or inhibited by a ROS1 inhibitor. In one embodiment, at least one biological activity of the PPFIBP1-ROS1 fusion polypeptide is reduced or inhibited by a PPFIBP1 inhibitor. In one embodiment, at least one biological activity of the PPFIBP1-ROS1 fusion polypeptide is reduced or inhibited by a ROS1 inhibitor, e.g., Ganetespib; Crizotinib; TAE684; a dual ALK and ROS1 inhibitor.

In yet other embodiments, the PPFIBP1-ROS1 fusion polypeptide is encoded by a nucleic acid molecule described herein. In one embodiment, the PPFIBP1-ROS1 fusion polypeptide is encoded by an in-frame fusion of intron 9 of PPFIBP1 with intron 34 of ROS1 (e.g., a sequence on chromosome 12 and a sequence on chromosome 6). In another embodiment, the PPFIBP1-ROS1 fusion polypeptide includes an amino acid sequence encoded by a nucleotide sequence comprising a fusion junction between the PPFIBP1 transcript and the ROS1 transcript.

In certain embodiments, the PPFIBP1-ROS1 fusion polypeptide comprises one or more of encoded exons 1-9 from PPFIBP1 and one or more of encoded exons 35-43 of ROS1. In certain embodiments, the PPFIBP1-ROS1 fusion polypeptide comprises at least 1, 2, 3, 4, 5, 6, 7, 8, 9 or more encoded exons of PPFIBP1 and at least at least 1, 2, 3, 4, 5, 6, 7, 8, 9 or more encoded exons of ROS1. In certain embodiments, the PPFIBP1-ROS1 fusion polypeptide comprises a fusion of encoded exon 9 from PPFIBP1 and encoded exon 35 from ROS1 (or a fragment thereof). In other embodiments, the fusion comprises at least 1, 2, 3, 4, 5, 6, 7, 8, 9 or more encoded exons of PPFIBP1; and at least 1, 2, 3, 4, 5, 6, 7, 8, 9 or more encoded exons of ROS1. In certain embodiments, the PPFIBP1-ROS1 fusion polypeptide comprises encoded exons 1-9 from PPFIBP1 and exons 35-43 of ROS1. In certain embodiments, the 5′ PPFIBP1-3′ ROS1 fusion polypeptide comprises a fusion junction of the sequence of exon 9 from PPFIBP1 and the sequence of exon 35 from ROS1.

In certain embodiments, the PPFIBP1-ROS1 fusion comprises the amino acid sequence corresponding to exon 9 or a fragment thereof from PPFIBP1, and the amino acid sequence corresponding to exon 35 or a fragment thereof from ROS1 (e.g., as shown in SEQ ID NO:20 and SEQ ID NO: 12). In one embodiment, the PPFIBP1-ROS1 fusion comprises at least 5, 10, 15, 20 or more amino acids from exon 9 of PPFIBP1 (e.g., from the amino acid sequence of PPFIBP1 preceding the fusion junction with ROS1, e.g., of the PPFIBP1 sequence shown in SEQ ID NO:20), and at least 5, 10, 15, 20 or more amino acids from exon 35 of ROS1 (e.g., from the amino acid sequence of ROS1 following the fusion junction with PPFIBP1. e.g., of the ROS1 sequence shown in SEQ ID NO:12).

In one embodiment, the PPFIBP1-ROS1 fusion polypeptide includes a tyrosine kinase domain or a functional fragment thereof. In a related aspect, the invention features PPFIBP1-ROS1 fusion polypeptide or fragments operatively linked to heterologous polypeptides to form fusion proteins.

In another embodiment, the PPFIBP1-ROS1 fusion polypeptide or fragment is a peptide, e.g., an immunogenic peptide or protein. This peptide or protein contains a fusion junction described herein. Such immunogenic peptides or proteins can be used to raise antibodies specific to the fusion protein. In other embodiments, such immunogenic peptides or proteins can be used for vaccine preparation. The vaccine preparation can include other components, e.g., an adjuvant.

In another aspect, the invention features antibody molecules that bind to a PPFIBP1-ROS1 fusion polypeptide or fragment described herein. In embodiments, the antibody can distinguish wild type ROS1 (or PPFIBP1) from PPFIBP1-ROS1.

### Detection Reagents and Detection of Mutations

In another aspect, the invention features a detection reagent, e.g., a purified or an isolated preparation thereof. Detection reagents can distinguish a nucleic acid, or protein sequence, having a breakpoint, e.g., a PPFIBP1-ROS1 breakpoint; from a reference sequence. In one embodiment, the detection reagent detects (e.g., specifically detects) a PPFIBP1-ROS1 fusion nucleic acid or a polypeptide, e.g., distinguishes a wild type ROS1 or another ROS1 fusion (or PPFIBP1) from a PPFIBP1-ROS1 nucleic acid (e.g., as described herein in SEQ ID NO: 19 and SEQ ID NO: 11); or a PPFIBP1-ROS1 polypeptide (e.g., as described herein in SEQ ID NO:20 and SEQ ID NO:12).

Detection reagents, e.g., nucleic acid-based detection reagents, can be used to identify mutations in a target nucleic acid, e.g., DNA, e.g., genomic DNA or cDNA, or RNA, e.g., in a sample, e.g., a sample of nucleic acid derived from a neoplastic or tumor cell, e.g., a melanocytic neoplasm, melanoma or metastatic cell. Detection reagents, e.g., antibody-based detection reagents, can be used to identify mutations in a target protein, e.g., in a sample, e.g., a sample of protein derived from, or produced by, a neoplastic or tumor cell, e.g., a melanocytic neoplasm, melanoma or metastatic cell.

### Method of Treatment

In a related aspect, a method of inhibiting, reducing, or treating a hyperproliferative disorder, e.g., a neoplasm (including benign, pre-malignant or malignant (e.g., a cancer), in a subject is provided. The method includes administering to the subject a preselected therapeutic agent, e.g., an anti-cancer agent (e.g., a kinase inhibitor), as a single agent, or in combination, in an amount sufficient to reduce, inhibit or treat the activity or expression of PPFIBP1-ROS1 (e.g., a PPFIBP1-ROS1 fusion described herein), thereby inhibiting, reducing, or treating the hyperproliferative disorder in the subject.

In one embodiment, the subject treated has a PPFIBP1-ROS1 fusion; e.g., the subject has a tumor or cancer harboring a PPFIBP1-ROS1 fusion. In other embodiments, the subject has been previously identified as having a PPFIBP1-ROS1 fusion. In yet other embodiments, the subject has been previously identified as being likely or unlikely to respond to treatment with a protein kinase inhibitor. e.g., a subject that has previously participated in a clinical trial. In other embodiments, the subject has been previously identified as being likely or unlikely to respond to treatment with a protein kinase inhibitor, based on the presence of the PPFIBP1-ROS1 fusion. In one embodiment, the subject is a mammal, e.g., a human. In one embodiment, the subject has, or at risk of having a cancer particularly melanoma at any stage of disease, for example atypical melanocytic neoplasm. In other embodiments, the subject is a patient, e.g., a cancer patient.

In certain embodiments, the neoplasm or neoplastic cell is a benign, pre-malignant, malignant (cancer) or metastasis. In certain embodiments, the cancer is a solid tumor, a soft tissue tumor, or a metastatic lesion.

In one embodiment, the neoplasm or neoplastic cell is a benign, pre-malignant, malignant (cancer) or metastasis. In certain embodiments, the cancer is a solid tumor, a soft tissue tumor, or a metastatic lesion. In one embodiment, the cancer is a carcinoma. In one embodiment, the cancer is an adenocarcinoma. In an embodiment, the cancer is a lung adenocarcinoma. In one embodiment, the cancer is chosen from a lung cancer, a pancreatic cancer, melanoma, a colorectal cancer, an esophageal-gastric cancer, a thyroid cancer, or an adenocarcinoma. In one embodiment, the cancer is a lung adenocarcinoma. In other embodiment, the lung cancer is chosen from one or more of the following: non-small cell lung cancer (NSCLC), small cell lung cancer (SCLC), squamous cell carcinoma (SCC), adenocarcinoma of the lung, bronchogenic carcinoma, or a combination thereof. In one embodiment, the lung cancer is NSCLC or SCC.

In one embodiment, the anti-cancer agent is a ROS1 inhibitor. In one embodiment, the anti-cancer agent is a PPFIBP1 inhibitor. In one embodiment, the anti-cancer agent is a kinase inhibitor. In one embodiment, the anti-cancer agent is a ROS1 inhibitor, e.g., Ganetespib; Crizotinib; TAE684; AP26113, X-276, X-376, X-396, CH5424802 (AF-802), GSK1838705, ASP3026, PHA-E429, CRL151104; and additional examples of kinase inhibitors are described in de la Bellacasa R. P. et al, Transl Lung Cancer Res 2013; 2(2):72-86.

### TPM3-ROS1 Fusions

In one embodiment, a fusion includes an in-frame fusion of an exon of tropomyosin 3 (TPM3), e.g., one more exons of TPM3 (e.g., one or more of exons 1-3 of TPM3) or a fragment thereof, and an exon of C-Ros oncogene 1 (ROS1), e.g., one or more exons of a ROS1 (e.g., one or more of exons 36-43 of ROS1) or a fragment thereof. For example, the TPM3-ROS1 fusion can include an in-frame fusion within an intron of TPM3 (e.g., intron 3) or a fragment thereof, with an intron of ROS1 (e.g., intron 35) or a fragment thereof. In one embodiment, the fusion of the TPM3-ROS1 fusion comprises the nucleotide sequence of: chromosome 1 at one or more of a nucleotide (plus or minus 10, 20, 30, 50, 60, 70, 80, 100 or more nucleotides) and chromosome 6 at one or more of a nucleotide (plus or minus 10, 20, 30, 50, 60, 70, 80, 100 or more nucleotides). In one embodiment, the TPM3-ROS1 fusion is a translocation, e.g., a translocation of a portion of chromosome 1 and a portion of chromosome 6.

In certain embodiments, the TPM3-ROS1 fusion is in a 5′-TPM3 to 3′-ROS1 configuration (also referred to herein as “5′-TPM3-ROS1-3′).” The term “fusion” or “fusion molecule” can refer to a polypeptide or a nucleic acid fusion, depending on the context. It may include a full-length sequence of a fusion or a fragment thereof, e.g., a fusion junction (e.g., a fragment including a portion of TPM3 and a portion of ROS1, e.g., a portion of the TPM3-ROS1 fusion described herein). In one embodiment, the TPM3-ROS1 fusion polypeptide includes a fragment of the amino acid sequence shown in SEQ ID NO: 10 and a fragment of the amino acid sequence shown in SEQ ID NO: 12, or an amino acid sequence substantially identical thereto. In another embodiment, the TPM3-ROS1 fusion nucleic acid includes a fragment of the nucleotide sequence shown in SEQ ID NO:9 and a fragment of the nucleotide sequence shown in SEQ ID NO: 1, or a nucleotide sequence substantially identical thereto. In one embodiment, the TPM3-ROS1 fusion polypeptide comprises sufficient TPM3 and sufficient ROS1 sequence such that the 5′ TPM3-3′ ROS1 fusion has kinase activity, e.g., has elevated activity, e.g., tyrosine kinase activity. The fusion causes activation in the cells harboring it of oncogenic signaling pathways.

In certain embodiments, the TPM3-ROS1 fusion comprises one or more (or all of) exons 1-3 from TPM3 and one or more (or all of) exons 36-43 of ROS1 (e.g., one or more of the exons shown in SEQ ID NO:9 and SEQ ID NO:11. In another embodiment, the TPM3-ROS1 fusion comprises one or more (or all of) exons 1-3 of TPM3 and one or more (or all of) exons 36-43 of ROS1. In certain embodiments, the TPM3-ROS1 fusion comprises at least 1, 2, 3 or more exons (or encoded exons) from TPM3 and at least 1, 2, 3, 4, 5, 6, 7, 8 or more exons (or encoded exons) from ROS1 (e.g., from the TPM3 and ROS1 sequences shown in SEQ ID NO:9 and SEQ ID NO: 10 and SEQ ID NO: 11 and SEQ ID NO:12.

In certain embodiments, the TPM3-ROS1 fusion comprises exons 1-3 or a fragment thereof from TPM3, and exons 36-43 or a fragment thereof from ROS1 (e.g., as shown in SEQ ID NO:9 and SEQ ID NO: 11). In one embodiment, the TPM3-ROS1 fusion comprises at least 5, 10, 15, 20, 30, 40, 50 or more amino acids from exons 1-3 of TPM3 (e.g., from the amino acid sequence of TPM3 as shown in SEQ ID NO:10 (e.g., from the amino acid sequence of TPM3 preceding the fusion junction with ROS1, and at least 5, 10, 15, 20, 30, 40, 50 or more amino acids from exons 36-43 of ROS1 (e.g., from the amino acid sequence of ROS1 as shown in SEQ ID NO: 12). In another embodiment, the TPM3-ROS1 fusion comprises at least 6, 12, 15, 20, 25, 50, 75, 100 or more nucleotides from exons 1-3 of TPM3 (e.g., from the nucleotide sequence of TPM3 as shown in SEQ ID NO:9 (e.g., from the nucleotide sequence of TPM3 preceding the fusion junction with ROS1); and at least 6, 12, 15, 20, 25, 50, 75, 100 or more nucleotides from exons 36-43 of ROS1 (e.g., from the nucleotide sequence of ROS1 as shown in SEQ ID NO: 11).

### TPM3-ROS1 Nucleic Acid Molecules

In one aspect, the invention features a nucleic acid molecule (e.g., an isolated or purified) nucleic acid molecule that includes a fragment of a TPM3 gene and a fragment of a ROS1 gene. In one embodiment, the nucleotide sequence encodes a TPM3-ROS1 fusion polypeptide that includes a tyrosine kinase domain or a functional fragment thereof. In another embodiment, the nucleotide sequence encodes a fragment of the ROS1 polypeptide including the amino acid sequence of SEQ ID NO: 12 or a fragment thereof, or a sequence substantially identical thereto. In other embodiments, the nucleic acid molecule includes a fragment of the TPM3 gene encoding the amino acid sequence of SEQ ID NO: 10 or a fragment thereof, or a sequence substantially identical thereto. In yet other embodiments, the nucleic acid molecule includes a nucleotide sequence encoding the amino acid sequence shown in SEQ ID NO: 10, or a fragment thereof, and the amino acid sequence shown in SEQ ID NO: 12 or a fragment thereof, or a sequence substantially identical thereto.

In one embodiment, the nucleic acid molecule includes a fusion, e.g., an in-frame fusion, between an intron of TPM3 (e.g., intron 3, or a fragment thereof), and an intron of ROS1 (e.g., intron 35, or a fragment thereof). The TPM3-ROS1 fusion can comprise a fusion of the nucleotide sequence of: chromosome 1 at one or more of a nucleotide (plus or minus 10, 20, 30, 50, 60, 70, 80, 100 nucleotides) and chromosome 6 at one or more of a nucleotide (plus or minus 10, 20, 30, 50, 60, 70, 80, 100 nucleotides), or a fragment thereof. In one embodiment, the TPM3-ROS1 fusion comprises a fusion of the nucleotide sequence of: chromosome 1 at one or more of a nucleotide (plus or minus 10, 20, 30, 50, 60, 70, 80, 100 nucleotides) and chromosome 6 at one or more of a nucleotide (plus or minus 10, 20, 30, 50, 60, 70, 80, 100 nucleotides), or a fragment thereof.

In another embodiment, the TPM3-ROS1 fusion comprises a nucleotide sequence (e.g., a fragment of a nucleotide sequence) shown in SEQ ID NO:9 and a nucleotide sequence (e.g., a fragment of a nucleotide sequence) shown in SEQ ID NO:11, or a fragment of the fusion. In one embodiment, the TPM3-ROS1 fusion comprises a nucleotide sequence substantially identical to the nucleotide sequence (e.g., a fragment of a nucleotide sequence) shown in SEQ ID NO:9 and the nucleotide sequence (e.g., a fragment of a nucleotide sequence) shown SEQ ID NO: 11, or a fragment of the fusion. In one embodiment, the TPM3-ROS1 fusion comprises a nucleotide sequence at least 70%, at least 75%, at least 80%, at least 85%, at least 90%, at least 95%, at least 99%, at least 99.5 or greater, identical to the nucleotide sequence (e.g., a fragment of a nucleotide sequence) shown in SEQ ID NO:9 and to the nucleotide sequence (e.g., a fragment of a nucleotide sequence) shown in SEQ ID NO: 11. In one embodiment, the TPM3-ROS1 fusion comprises a nucleotide sequence containing at least 25, 50, 100, 150, 200, 500, 1000, 1500, 2000, 2500, 3000, or more nucleotides of the nucleotide sequence shown in SEQ ID NO:9 and a nucleotide sequence containing at least 25, 50, 100, 150, 200, 500, 1000, 1500, 2000, 2500, 3000, or more nucleotides of the nucleotide sequence shown in SEQ ID NO: 11. In one embodiment, the TPM3-ROS1 fusion comprises a nucleotide sequence containing at least 25, 50, 100, 150, 200, 500, 1000, 1500, 2000, 2500, 3000, or more contiguous nucleotides of the nucleotide sequence shown in SEQ ID NO:9 and a nucleotide sequence containing at least 25, 50, 100, 150, 200, 500, 1000, 1500, 2000, 2500, 3000, or more nucleotides of the nucleotide sequence shown in SEQ ID NO: 11.

In another embodiment, the nucleic acid molecule includes a fusion, e.g., an in-frame fusion, of at least 1, 2, 3 or more exons of TPM3 or a fragment thereof (e.g., one or more of exons 1-3 of TPM3 or a fragment thereof), and at least 1, 2, 3, 4, 5, 6, 7, 8 or more exons of ROS1 or a fragment thereof (e.g., one or more of exons 36-43 of ROS1 or a fragment thereof). In yet other embodiments, the nucleic acid molecule includes a fragment the nucleotide sequence shown in SEQ ID NO:9 and a fragment of the nucleotide sequence shown in SEQ ID NO:11 or a fragment of the fusion, or a sequence substantially identical thereto.

In one embodiment, the nucleic acid molecule is complementary to at least a portion of a nucleotide sequence disclosed herein, e.g., is capable of hybridizing under a stringency condition described herein to SEQ ID NO:9 and/or SEQ ID NO: 11, or a fragment thereof. In yet another embodiment, the nucleic acid molecule hybridizes to a nucleotide sequence that is complementary to at least a portion of a nucleotide sequence disclosed herein, e.g., is capable of hybridizing under a stringency condition to a nucleotide sequence complementary to SEQ ID NO:9 and/or SEQ ID NO: 11, or a fragment thereof. The nucleotide sequence of a cDNA encoding an exemplary 5′ TPM3-3′ ROS1 fusion is shown in at least exon 20 (e.g., exons 1-3) of SEQ ID NO:9 and at least exon 36 (e.g., exons 36-43) of SEQ ID NO: 11, and the predicted amino acid sequence is shown in the corresponding encoded exons of SEQ ID NO: 10 and the corresponding encoded exons of SEQ ID NO: 12, respectively.

In an embodiment the TPM3-ROS1 nucleic acid molecule comprises sufficient TPM3 and sufficient ROS1 sequence such that the encoded 5′ TPM3-3′ ROS1 fusion has kinase activity, e.g., has elevated activity. In certain embodiments, the 5′ TPM3-3′ ROS1 fusion comprises exons 1-3 from TPM3 and exons 36-43 from ROS1. In certain embodiments, the TPM3-ROS1 fusion comprises at least 1, 2, 3 or more exons from TPM3 and at least at least 1, 2, 3, 4, 5, 6, 7, 8 or more exons of ROS1. In certain embodiments, the TPM3-ROS1 fusion comprises a fusion of exon 20 from TPM3 and exon 36 from ROS1. In another embodiment, the TPM3-ROS1 fusion comprises 1, 2, 3 or more exons of TPM3; and at least 1, 2, 3, 4, 5, 6, 7, 8 or more exons of ROS1.

In one embodiment, the nucleic acid molecule includes a nucleotide sequence that has an in-frame fusion of intron 3 of TPM3 (e.g., NM_{—}152263) with intron 35 of ROS1 (e.g., NM_{—}002944). In another embodiment, the nucleic acid molecule includes a nucleotide sequence that includes a breakpoint. For example, the nucleic acid molecule includes a nucleotide sequence that includes the fusion junction between the TPM3 gene and the ROS1 gene, e.g., the breakpoint between intron 3 of TPM3 and intron 35 of ROS1. In other embodiments, the nucleic acid molecules includes a nucleotide sequence of one or more of nucleotide of chromosome 1 coupled to (e.g., directly or indirectly juxtaposed to) one or more of nucleotide of chromosome 6. In one embodiment, the nucleic acid molecule includes the nucleotide sequence of: chromosome 1 at one or more of a nucleotide plus or minus 10, 20, 30, 40 50, 60, 80, 100, 150 nucleotides and chromosome 6 at one or more of a nucleotide plus or minus 10, 20, 30, 40 50, 60, 80, 100, 150 nucleotides (corresponding to the breakpoint of a TPM3-ROS1 fusion), or a fragment thereof, or a sequence substantially identical thereto. In one embodiment, the nucleic acid molecule is complementary to at least a portion of a nucleotide sequence disclosed herein, e.g., is capable of hybridizing under a stringency condition described herein to SEQ ID NO:9 and/or SEQ ID NO:11 or a fragment thereof. In yet other embodiment, the nucleic acid molecule hybridizes to a nucleotide sequence that is complementary to at least a portion of a nucleotide sequence disclosed herein, e.g., is capable of hybridizing under a stringency condition described herein to a nucleotide sequence complementary to SEQ ID NO:9 or SEQ ID NO: 11 or a fragment thereof.

In another embodiment, the TPM3-ROS1 fusion nucleic acid comprises at least 6, 12, 15, 20, 25, 50, 75, 100 or more nucleotides from exon 20 of TPM3 (e.g., from the nucleotide sequence of TPM3 preceding the fusion junction with ROS1, e.g., of the TPM3 sequence shown in SEQ ID NO:9), and at least 6, 12, 15, 20, 25, 50, 75, 100 or more nucleotides from exon 36 of ROS1 (e.g., from the nucleotide sequence of ROS1 following the fusion junction with TPM3, e.g., of the ROS1 sequence shown in SEQ ID NO: 1).

In other embodiments, the nucleic acid molecule includes a nucleotide sequence encoding a TPM3-ROS1 fusion polypeptide that includes a fragment of a TPM3 gene and a fragment of a ROS1 gene. In one embodiment, the nucleotide sequence encodes a TPM3-ROS1 fusion polypeptide that includes e.g., a tyrosine kinase domain or a functional fragment thereof. In yet other embodiments, the nucleic acid molecule includes a nucleotide sequence encoding the amino acid sequence (e.g., a fragment of the amino acid sequence) shown in SEQ ID NO: 10 and a nucleotide sequence encoding the amino acid sequence (e.g., a fragment of the amino acid sequence) shown in SEQ ID NO:12, or a fragment of the fusion, or a sequence substantially identical thereto. In one embodiment, the encoded TPM3-ROS1 fusion polypeptide includes a tyrosine kinase domain or a functional fragment thereof.

In one embodiment, the TPM3-ROS1 fusion includes the full sequence or a fragment of the nucleotide sequence shown in SEQ ID NO:21 and SEQ ID NO:94, or a nucleotide sequence substantially identical thereto. In another embodiment, the TPM3-ROS1 fusion polypeptide includes the full sequence or a fragment of the amino acid sequence shown in SEQ ID NO:22 and SEQ ID NO:95, or an amino acid sequence substantially identical thereto, for example at least 70% identical or 80% identical or 90% identical or even more. In a related aspect, the invention features nucleic acid constructs that include the TPM3-ROS1 nucleic acid molecules described herein. In certain embodiments, the nucleic acid molecules are operatively linked to a native or a heterologous regulatory sequence. Also included are vectors and host cells that include the TPM3-ROS1 nucleic acid molecules described herein, e.g., vectors and host cells suitable for producing the nucleic acid molecules and polypeptides described herein.

In a related aspect, methods of producing the nucleic acid molecules and polypeptides described herein are also described.

In another aspect, the invention features nucleic acid molecules that reduce or inhibit the expression of a nucleic acid molecule that encodes a TPM3-ROS1 fusion described herein. Examples of such nucleic acid molecules include, for example, antisense molecules, ribozymes, RNAi, triple helix molecules that hybridize to a nucleic acid encoding TPM3-ROS1, or a transcription regulatory region of TPM3-ROS1, and blocks or reduces mRNA expression of TPM3-ROS1.

### Nucleic Acid Detection and Capturing Reagents

The invention also features a nucleic acid molecule, e.g., nucleic acid fragment, suitable as probe, primer, bait or library member that includes, flanks, hybridizes to, which are useful for identifying, or are otherwise based on, the TPM3-ROS1 fusions described herein. In certain embodiments, the probe, primer or bait molecule is an oligonucleotide that allows capture, detection or isolation of a TPM3-ROS1 fusion nucleic acid molecule described herein. The oligonucleotide can comprise a nucleotide sequence substantially complementary to a fragment of the TPM3-ROS1 fusion nucleic acid molecules described herein. The sequence identity between the nucleic acid fragment, e.g., the oligonucleotide, and the target TPM3-ROS1 sequence need not be exact, so long as the sequences are sufficiently complementary to allow the capture, detection or isolation of the target sequence. In one embodiment, the nucleic acid fragment is a probe or primer that includes an oligonucleotide between about 5 and 25, e.g., between 10 and 20, or 10 and 15 nucleotides in length. In other embodiments, the nucleic acid fragment is a bait that includes an oligonucleotide between about 100 to 300 nucleotides, 130 and 230 nucleotides, or 150 and 200 nucleotides, in length.

In one embodiment, the nucleic acid fragment can be used to identify or capture, e.g., by hybridization, a TPM3-ROS1 fusion. For example, the nucleic acid fragment can be a probe, a primer, or a bait, for use in identifying or capturing, e.g., by hybridization, a TPM3-ROS1 fusion described herein. In one embodiment, the nucleic acid fragment can be useful for identifying or capturing a TPM3-ROS1 breakpoint. e.g., the nucleotide sequence of: chromosome 1 at nucleotide plus or minus 10, 20, 30, 40, 50, 60, 80, 100, 150 nucleotides and chromosome 6 at nucleotide plus or minus 10, 20, 30, 40 50, 60, 80, 100, 150 nucleotides.

In one embodiment, the nucleic acid fragment hybridizes to a nucleotide sequence within a chromosomal rearrangement that creates an in-frame fusion of intron 3 of TPM3 with intron 35 of ROS1. In one embodiment, the nucleic acid fragment hybridizes to a nucleotide sequence in the region In other embodiments, the nucleic acid molecules includes a nucleotide sequence in the region of a nucleotide sequence of chromosome 1 coupled to (e.g., juxtaposed to) nucleotides in the region of a nucleotide sequence of chromosome 6. In one embodiment, the nucleic acid fragment hybridizes to a nucleotide sequence that includes a breakpoint, e.g., the nucleotide sequence of: chromosome 1 at nucleotide plus or minus 10, 20, 30, 40, 50, 60, 80, 100, 150 or more nucleotides and chromosome 6 at nucleotide plus or minus 10, 20, 30, 40, 50, 60, 80, 100, 150 or more nucleotides. For example, the nucleic acid fragment can hybridize to a nucleotide sequence that includes the fusion junction between the TPM3 gene and the ROS1 gene, e.g., a nucleotide sequence that includes a portion of a nucleotide sequence within intron 3 of a TPM3 gene and intron 35 of a ROS1 gene.

In another embodiment, the nucleic acid fragment hybridizes to a nucleotide sequence that comprises at least 6, 12, 15, 20, 25, 50, 75, 100, 150 or more nucleotides from exon 20 of TPM3 (e.g., from the nucleotide sequence of TPM3 preceding the fusion junction with ROS1, e.g., of the TPM3 sequence shown in SEQ ID NO:9), and at least 6, 12, 15, 20, 25, 50, 75, 100, 150 or more nucleotides from exon 36 of ROS1 (e.g., from the nucleotide sequence of ROS1 following the fusion junction with TPM3, e.g., of the ROS1 sequence shown in SEQ ID NO: 11).

The probes or primers described herein can be used, for example, for FISH detection or PCR amplification. In one exemplary embodiment where detection is based on PCR, amplification of the TPM3-ROS1 fusion junction fusion junction can be performed using a primer or a primer pair, e.g., for amplifying a sequence flanking the fusion junctions described herein, e.g., the mutations or the junction of a chromosomal rearrangement described herein, e.g. TPM3-ROS1.

In one embodiment, a pair of isolated oligonucleotide primers can amplify a region containing or adjacent to a position in the TPM3-ROS1 fusion. For example, forward primers can be designed to hybridize to a nucleotide sequence within TPM3 genomic or mRNA sequence (e.g., a nucleotide sequence within exon 20 of TPM3 of SEQ ID NO:9), and the reverse primers can be designed to hybridize to a nucleotide sequence of ROS1 (e.g., a nucleotide sequence within exon 36 of ROS1, of SEQ ID NO: 11).

In another embodiment, the nucleic acid fragments can be used to identify, e.g., by hybridization, a TPM3-ROS1 fusion. In one embodiment, the nucleic acid fragment hybridizes to a nucleotide sequence that includes a fusion junction between the TPM3 transcript and the ROS1 transcript.

In other embodiments, the nucleic acid fragment includes a bait that comprises a nucleotide sequence that hybridizes to a TPM3-ROS1 fusion nucleic acid molecule described herein, and thereby allows the capture or isolation said nucleic acid molecule. In one embodiment, a bait is suitable for solution phase hybridization. In other embodiments, a bait includes a binding entity, e.g., an affinity tag, that allows capture and separation, e.g., by binding to a binding entity, of a hybrid formed by a bait and a nucleic acid hybridized to the bait.

In other embodiments, the nucleic acid fragment includes a library member comprising a TPM3-ROS1 nucleic acid molecule described herein. In one embodiment, the library member includes a rearrangement that results in a TPM3-ROS1 fusion described herein.

The nucleic acid fragment can be detectably labeled with, e.g., a radiolabel, a fluorescent label, a bioluminescent label, a chemiluminescent label, an enzyme label, a binding pair label, or can include an affinity tag; a tag, or identifier (e.g., an adaptor, barcode or other sequence identifier).

### TPM3-ROS1 Fusion Polypeptides

In another embodiment, the TPM3-ROS1 fusion comprises an amino acid sequence (e.g., a fragment of the amino acid sequence) shown in SEQ ID NO: 10 and an amino acid sequence (e.g., a fragment of the amino acid sequence) shown in SEQ ID NO: 12, or a fragment of the fusion. In one embodiment, the TPM3-ROS1 fusion comprises an amino acid sequence substantially identical to the amino acid sequence (e.g., a fragment of the amino acid sequence) shown in SEQ ID NO: 10 and the amino acid sequence (e.g., a fragment of the amino acid sequence) shown in SEQ ID NO:12, or a fragment thereof. In one embodiment, the TPM3-ROS1 fusion comprises an amino acid sequence at least 70%, at least 75%, at least 80%, at least 85%, at least 90%, at least 95%, at least 99%, at least 99.5 or greater, identical to the amino acid sequence (e.g., a fragment of the amino acid sequence) shown in SEQ ID NO: 10 and the amino acid sequence (e.g., a fragment of the amino acid sequence) shown in SEQ ID NO:12. In one embodiment, the TPM3-ROS1 fusion comprises a sequence containing at least 10, 20, 50, 100, 500, 600, 700, 800, 900, 1000, or more amino acids of the amino acid sequence shown in SEQ ID NO:10 and SEQ ID NO:12. In one embodiment, the TPM3-ROS1 fusion comprises an amino acid sequence containing at least 5, 10, 20, 50, 100, 500, 600, 700, 800, 900, 1000, or more contiguous amino acids of the amino acid sequence shown in SEQ ID NO:10 and at least 5, 10, 20, 50, 100, 500, 600, 700, 800, 900, 1000, or more contiguous amino acids of the amino acid sequence shown in SEQ ID NO:12. In one embodiment, the 5′ TPM3-3′ ROS1 fusion polypeptide includes a receptor tyrosine kinase domain or a functional fragment thereof. In an embodiment, the 5′TPM3-3′ROS1 fusion polypeptide comprises sufficient ROS1 and sufficient TPM3 sequence such that it has kinase activity, e.g., has elevated activity.

In another aspect, the invention features a TPM3-ROS1 fusion polypeptide (e.g., a purified TPM3-ROS1 fusion polypeptide), a biologically active or antigenic fragment thereof, as well as reagents (e.g., antibody molecules that bind to a TPM3-ROS1 fusion polypeptide), methods for modulating a TPM3-ROS1 polypeptide activity and detection of a TPM3-ROS1 polypeptide.

In one embodiment, the TPM3-ROS1 fusion polypeptide has at least one biological activity. In one embodiment, at least one biological activity of the TPM3-ROS1 fusion polypeptide is reduced or inhibited by an anti-cancer drug, e.g., a TPM3 inhibitor, a ROS1 inhibitor. In one embodiment, at least one biological activity of the TPM3-ROS1 fusion polypeptide is reduced or inhibited by a ROS1 inhibitor. In one embodiment, at least one biological activity of the TPM3-ROS1 fusion polypeptide is reduced or inhibited by a TPM3 inhibitor. In one embodiment, at least one biological activity of the TPM3-ROS1 fusion polypeptide is reduced or inhibited by a ROS1 inhibitor, e.g., Ganetespib; Crizotinib; TAE684; a dual ALK and ROS1 inhibitor.

In yet other embodiments, the TPM3-ROS1 fusion polypeptide is encoded by a nucleic acid molecule described herein. In one embodiment, the TPM3-ROS1 fusion polypeptide is encoded by an in-frame fusion of intron 3 of TPM3 with intron 35 of ROS1 (e.g., a sequence on chromosome 1 and a sequence on chromosome 6). In another embodiment, the TPM3-ROS1 fusion polypeptide includes an amino acid sequence encoded by a nucleotide sequence comprising a fusion junction between the TPM3 transcript and the ROS1 transcript.

In certain embodiments, the TPM3-ROS1 fusion polypeptide comprises one or more of encoded exons 1-3 from TPM3 and one or more of encoded exons 36-43 of ROS1. In certain embodiments, the TPM3-ROS1 fusion polypeptide comprises at least 1, 2, 3 or more encoded exons of TPM3 and at least at least 1, 2, 3, 4, 5, 6, 7, 8 or more encoded exons of ROS1. In certain embodiments, the TPM3-ROS1 fusion polypeptide comprises a fusion of encoded exon 20 from TPM3 and encoded exon 36 from ROS1 (or a fragment thereof). In other embodiments, the fusion comprises at least 1, 2, 3 or more encoded exons of TPM3; and at least 1, 2, 3, 4, 5, 6, 7, 8 or more encoded exons of ROS1. In certain embodiments, the TPM3-ROS1 fusion polypeptide comprises encoded exons 1-3 from TPM3 and exons 36-43 of ROS1. In certain embodiments, the 5′ TPM3-3′ ROS1 fusion polypeptide comprises a fusion junction of the sequence of exon 20 from TPM3 and the sequence of exon 36 from ROS1.

In certain embodiments, the TPM3-ROS1 fusion comprises the amino acid sequence corresponding to exon 20 or a fragment thereof from TPM3, and the amino acid sequence corresponding to exon 36 or a fragment thereof from ROS1 (e.g., as shown in SEQ ID NO:10 and SEQ ID NO:12). In one embodiment, the TPM3-ROS1 fusion comprises at least 5, 10, 15, 20 or more amino acids from exon 20 of TPM3 (e.g., from the amino acid sequence of TPM3 preceding the fusion junction with ROS1, e.g., of the TPM3 sequence shown in SEQ ID NO:10), and at least 5, 10, 15, 20 or more amino acids from exon 36 of ROS1 (e.g., from the amino acid sequence of ROS1 following the fusion junction with TPM3, e.g., of the ROS1 sequence shown in SEQ ID NO: 12).

In one embodiment, the TPM3-ROS1 fusion polypeptide includes a tyrosine kinase domain or a functional fragment thereof. In a related aspect, the invention features TPM3-ROS1 fusion polypeptide or fragments operatively linked to heterologous polypeptides to form fusion proteins. In particular embodiments the fusion protein has a constitutively active kinase domain, or in any event a more active kinase than a normal cell containing only the intact wild-type genes and not the TPM3-ROS1 fusion.

In another embodiment, the TPM3-ROS1 fusion polypeptide or fragment is a peptide, e.g., an immunogenic peptide or protein. This peptide or protein contains a fusion junction described herein. Such immunogenic peptides or proteins can be used to raise antibodies specific to the fusion protein. In other embodiments, such immunogenic peptides or proteins can be used for vaccine preparation. The vaccine preparation can include other components, e.g., an adjuvant.

In another aspect, the invention features antibody molecules that bind to a TPM3-ROS1 fusion polypeptide or fragment described herein. In embodiments, the antibody can distinguish wild type ROS1 (or TPM3) from TPM3-ROS1.

### Detection Reagents and Detection of Mutations

In another aspect, the invention features a detection reagent, e.g., a purified or an isolated preparation thereof. Detection reagents can distinguish a nucleic acid, or protein sequence, having a breakpoint, e.g., a TPM3-ROS1 breakpoint; from a reference sequence. In one embodiment, the detection reagent detects (e.g., specifically detects) a TPM3-ROS1 fusion nucleic acid or a polypeptide, e.g., distinguishes a wild type ROS1 or another ROS1 fusion (or TPM3) from a TPM3-ROS1 nucleic acid (e.g., as described herein in SEQ ID NO:9 and SEQ ID NO:11); or a TPM3-ROS1 polypeptide (e.g., as described herein in SEQ ID NO: 10 and SEQ ID NO: 12).

Detection reagents, e.g., nucleic acid-based detection reagents, can be used to identify mutations in a target nucleic acid, e.g., DNA, e.g., genomic DNA or cDNA, or RNA. e.g., in a sample, e.g., a sample of nucleic acid derived from a neoplastic or tumor cell, e.g., a melanocytic neoplasm, melanoma or metastatic cell. Detection reagents, e.g., antibody-based detection reagents, can be used to identify mutations in a target protein, e.g., in a sample, e.g., a sample of protein derived from, or produced by, a neoplastic or tumor cell, e.g., a melanocytic neoplasm, melanoma or metastatic cell.

### Method of Treatment

In a related aspect, a method of inhibiting, reducing, or treating a hyperproliferative disorder, e.g., a neoplasm (including benign, pre-malignant or malignant (e.g., a cancer), in a subject is provided. The method includes administering to the subject a preselected therapeutic agent, e.g., an anti-cancer agent (e.g., a kinase inhibitor), as a single agent, or in combination, in an amount sufficient to reduce, inhibit or treat the activity or expression of TPM3-ROS1 (e.g., a TPM3-ROS1 fusion described herein), thereby inhibiting, reducing, or treating the hyperproliferative disorder in the subject.

In one embodiment, the subject treated has a TPM3-ROS1 fusion; e.g., the subject has a tumor or cancer harboring a TPM3-ROS1 fusion. In other embodiments, the subject has been previously identified as having a TPM3-ROS1 fusion. In yet other embodiments, the subject has been previously identified as being likely or unlikely to respond to treatment with a protein kinase inhibitor, e.g., a subject that has previously participated in a clinical trial. In other embodiments, the subject has been previously identified as being likely or unlikely to respond to treatment with a protein kinase inhibitor, based on the presence of the TPM3-ROS1 fusion. In one embodiment, the subject is a mammal, e.g., a human. In one embodiment, the subject has, or at risk of having a cancer at any stage of disease. In other embodiments, the subject is a patient, e.g., a cancer patient.

In certain embodiments, the neoplasm or neoplastic cell is a benign, pre-malignant, malignant (cancer) or metastasis. In certain embodiments, the cancer is a solid tumor, a soft tissue tumor, or a metastatic lesion.

In one embodiment, the neoplasm or neoplastic cell is a benign, pre-malignant, malignant (cancer) or metastasis. In certain embodiments, the cancer is a solid tumor, a soft tissue tumor, or a metastatic lesion. In one embodiment, the cancer is a carcinoma. In one embodiment, the cancer is an adenocarcinoma. In an embodiment, the cancer is a lung adenocarcinoma. In one embodiment, the cancer is chosen from a lung cancer, a pancreatic cancer, melanoma, a colorectal cancer, an esophageal-gastric cancer, a thyroid cancer, or an adenocarcinoma. In one embodiment, the cancer is a lung adenocarcinoma. In other embodiment, the lung cancer is chosen from one or more of the following: non-small cell lung cancer (NSCLC), small cell lung cancer (SCLC), squamous cell carcinoma (SCC), adenocarcinoma of the lung, bronchogenic carcinoma, or a combination thereof. In one embodiment, the lung cancer is NSCLC or SCC.

In one embodiment, the anti-cancer agent is a ROS1 inhibitor. In one embodiment, the anti-cancer agent is a TPM3 inhibitor. In one embodiment, the anti-cancer agent is a kinase inhibitor. In one embodiment, the anti-cancer agent is a ROS1 inhibitor, e.g., Ganetespib; Crizotinib; TAE684; AP26113, X-276, X-376, X-396, CH5424802 (AF-802), GSK1838705, ASP3026, PHA-E429, CRL151104; and additional examples of kinase inhibitors are described in de la Bellacasa R. P. et al, Transl Lung Cancer Res 2013; 2(2):72-86.

### ZCCHC8-ROS1 Fusions

In one embodiment, a fusion includes an in-frame fusion of an exon of zinc finger CCHC domain containing 8 protein (ZCCHC8), e.g., one more exons of ZCCHC8 (e.g., one or more of exons 1-2 of ZCCHC8) or a fragment thereof, and an exon of C-Ros oncogene 1 (ROS1), e.g., one or more exons of a ROS1 (e.g., one or more of exons 36-43 of ROS1) or a fragment thereof. For example, the ZCCHC8-ROS1 fusion can include an in-frame fusion within an intron of ZCCHC8 (e.g., intron 1) or a fragment thereof, with an intron of ROS1 (e.g., intron 35) or a fragment thereof. In one embodiment, the fusion of the ZCCHC8-ROS1 fusion comprises the nucleotide sequence of: chromosome 12 at one or more of a nucleotide (plus or minus 10, 20, 30, 50, 60, 70, 80, 100 or more nucleotides) and chromosome 6 at one or more of a nucleotide (plus or minus 10, 20, 30, 50, 60, 70, 80, 100 or more nucleotides). In one embodiment, the ZCCHC8-ROS1 fusion is a translocation, e.g., a translocation of a portion of chromosome 12 and a portion of chromosome 6.

In certain embodiments, the ZCCHC8-ROS1 fusion is in a 5′-ZCCHC8 to 3′-ROS1 configuration (also referred to herein as “5′-ZCCHC8-ROS1-3′).” The term “fusion” or “fusion molecule” can refer to a polypeptide or a nucleic acid fusion, depending on the context. It may include a full-length sequence of a fusion or a fragment thereof, e.g., a fusion junction (e.g., a fragment including a portion of ZCCHC8 and a portion of ROS1, e.g., a portion of the ZCCHC8-ROS1 fusion described herein). In one embodiment, the ZCCHC8-ROS1 fusion polypeptide includes a fragment of the amino acid sequence shown in SEQ ID NO:26 and a fragment of the amino acid sequence shown in SEQ ID NO: 12, or an amino acid sequence substantially identical thereto. In another embodiment, the ZCCHC8-ROS1 fusion nucleic acid includes a fragment of the nucleotide sequence shown in SEQ ID NO:25 and a fragment of the nucleotide sequence shown in SEQ ID NO: 11, or a nucleotide sequence substantially identical thereto. In one embodiment, the ZCCHC8-ROS1 fusion polypeptide comprises sufficient ZCCHC8 and sufficient ROS1 sequence such that the 5′ ZCCHC8-3′ ROS1 fusion has kinase activity, e.g., has elevated activity, e.g., tyrosine kinase activity the fusion causes activation in the cells harboring it of oncogenic signaling pathways.

In certain embodiments, the ZCCHC8-ROS1 fusion comprises one or more (or all of) exons 1-2 from ZCCHC8 and one or more (or all of) exons 36-43 of ROS1 (e.g., one or more of the exons shown in SEQ ID NO:25 and SEQ ID NO: 11. In another embodiment, the ZCCHC8-ROS1 fusion comprises one or more (or all of) exons 1-2 of ZCCHC8 and one or more (or all of) exons 36-43 of ROS1. In certain embodiments, the ZCCHC8-ROS1 fusion comprises at least 1, 2 or more exons (or encoded exons) from ZCCHC8 and at least 1, 2, 3, 4, 5, 6, 7, 8 or more exons (or encoded exons) from ROS1 (e.g., from the ZCCHC8 and ROS1 sequences shown in SEQ ID NO:25 and SEQ ID NO:26 and SEQ ID NO: 11 and SEQ ID NO: 12.

In certain embodiments, the ZCCHC8-ROS1 fusion comprises exons 1-2 or a fragment thereof from ZCCHC8, and exons 36-43 or a fragment thereof from ROS1 (e.g., as shown in SEQ ID NO:25 and SEQ ID NO: 11). In one embodiment, the ZCCHC8-ROS1 fusion comprises at least 5, 10, 15, 20, 30, 40, 50 or more amino acids from exons 1-2 of ZCCHC8 (e.g., from the amino acid sequence of ZCCHC8 as shown in SEQ ID NO:26 (e.g., from the amino acid sequence of ZCCHC8 preceding the fusion junction with ROS1, and at least 5, 10, 15, 20, 30, 40, 50 or more amino acids from exons 36-43 of ROS1 (e.g., from the amino acid sequence of ROS1 as shown in SEQ ID NO: 12). In another embodiment, the ZCCHC8-ROS1 fusion comprises at least 6, 12, 15, 20, 25, 50, 75, 100 or more nucleotides from exons 1-2 of ZCCHC8 (e.g., from the nucleotide sequence of ZCCHC8 as shown in SEQ ID NO:25 (e.g., from the nucleotide sequence of ZCCHC8 preceding the fusion junction with ROS1); and at least 6, 12, 15, 20, 25, 50, 75, 100 or more nucleotides from exons 36-43 of ROS1 (e.g., from the nucleotide sequence of ROS1 as shown in SEQ ID NO: 11).

### ZCCHC8-ROS1 Nucleic Acid Molecules

In one aspect, the invention features a nucleic acid molecule (e.g., an isolated or purified) nucleic acid molecule that includes a fragment of a ZCCHC8 gene and a fragment of a ROS1 gene. In one embodiment, the nucleotide sequence encodes a ZCCHC8-ROS1 fusion polypeptide that includes a tyrosine kinase domain or a functional fragment thereof. In another embodiment, the nucleotide sequence encodes a fragment of the ROS1 polypeptide including the amino acid sequence of SEQ ID NO: 12 or a fragment thereof, or a sequence substantially identical thereto. In other embodiments, the nucleic acid molecule includes a fragment of the ZCCHC8 gene encoding the amino acid sequence of SEQ ID NO:26 or a fragment thereof, or a sequence substantially identical thereto. In yet other embodiments, the nucleic acid molecule includes a nucleotide sequence encoding the amino acid sequence shown in SEQ ID NO:26, or a fragment thereof, and the amino acid sequence shown in SEQ ID NO: 12 or a fragment thereof, or a sequence substantially identical thereto.

In one embodiment, the nucleic acid molecule includes a fusion. e.g., an in-frame fusion, between an intron of ZCCHC8 (e.g., intron 1, or a fragment thereof), and an intron of ROS1 (e.g., intron 35, or a fragment thereof). The ZCCHC8-ROS1 fusion can comprise a fusion of the nucleotide sequence of: chromosome 12 at one or more of a nucleotide (plus or minus 10, 20, 30, 50, 60, 70, 80, 100 nucleotides) and chromosome 6 at one or more of a nucleotide (plus or minus 10, 20, 30, 50, 60, 70, 80, 100 nucleotides), or a fragment thereof. In one embodiment, the ZCCHC8-ROS1 fusion comprises a fusion of the nucleotide sequence of: chromosome 12 at one or more of a nucleotide (plus or minus 10, 20, 30, 50, 60, 70, 80, 100 nucleotides) and chromosome 6 at one or more of a nucleotide (plus or minus 10, 20, 30, 50, 60, 70, 80, 100 nucleotides), or a fragment thereof.

In another embodiment, the ZCCHC8-ROS1 fusion comprises a nucleotide sequence (e.g., a fragment of a nucleotide sequence) shown in SEQ ID NO:25 and a nucleotide sequence (e.g., a fragment of a nucleotide sequence) shown in SEQ ID NO: 11, or a fragment of the fusion. In one embodiment, the ZCCHC8-ROS1 fusion comprises a nucleotide sequence substantially identical to the nucleotide sequence (e.g., a fragment of a nucleotide sequence) shown in SEQ ID NO:25 and the nucleotide sequence (e.g., a fragment of a nucleotide sequence) shown SEQ ID NO:11, or a fragment of the fusion. In one embodiment, the ZCCHC8-ROS1 fusion comprises a nucleotide sequence at least 70%, at least 75%, at least 80%, at least 85%, at least 90%, at least 95%, at least 99%, at least 99.5 or greater, identical to the nucleotide sequence (e.g., a fragment of a nucleotide sequence) shown in SEQ ID NO:25 and to the nucleotide sequence (e.g., a fragment of a nucleotide sequence) shown in SEQ ID NO:11. In one embodiment, the ZCCHC8-ROS1 fusion comprises a nucleotide sequence containing at least 25, 50, 100, 150, 200, 500, 1000, 1500, 2000, 2500, 3000, or more nucleotides of the nucleotide sequence shown in SEQ ID NO:25 and a nucleotide sequence containing at least 25, 50, 100, 150, 200, 500, 1000, 1500, 2000, 2500, 3000, or more nucleotides of the nucleotide sequence shown in SEQ ID NO: 11. In one embodiment, the ZCCHC8-ROS1 fusion comprises a nucleotide sequence containing at least 25, 50, 100, 150, 200, 500, 1000, 1500, 2000, 2500, 3000, or more contiguous nucleotides of the nucleotide sequence shown in SEQ ID NO:25 and a nucleotide sequence containing at least 25, 50, 100, 150, 200, 500, 1000, 1500, 2000, 2500, 3000, or more nucleotides of the nucleotide sequence shown in SEQ ID NO:11.

In another embodiment, the nucleic acid molecule includes a fusion, e.g., an in-frame fusion, of at least 1, 2 or more exons of ZCCHC8 or a fragment thereof (e.g., one or more of exons 1-2 of ZCCHC8 or a fragment thereof), and at least 1, 2, 3, 4, 5, 6, 7, 8 or more exons of ROS1 or a fragment thereof (e.g., one or more of exons 36-43 of ROS1 or a fragment thereof). In yet other embodiments, the nucleic acid molecule includes a fragment the nucleotide sequence shown in SEQ ID NO:25 and a fragment of the nucleotide sequence shown in SEQ ID NO: 11 or a fragment of the fusion, or a sequence substantially identical thereto.

In one embodiment, the nucleic acid molecule is complementary to at least a portion of a nucleotide sequence disclosed herein, e.g., is capable of hybridizing under a stringency condition described herein to SEQ ID NO:25 and/or SEQ ID NO: 11, or a fragment thereof. In yet another embodiment, the nucleic acid molecule hybridizes to a nucleotide sequence that is complementary to at least a portion of a nucleotide sequence disclosed herein, e.g., is capable of hybridizing under a stringency condition to a nucleotide sequence complementary to SEQ ID NO:25 and/or SEQ ID NO: 11, or a fragment thereof. The nucleotide sequence of a cDNA encoding an exemplary 5′ ZCCHC8-3′ ROS1 fusion is shown in at least exons 1-2 (e.g., exons 1-2) of SEQ ID NO:25 and at least exon 36 (e.g., exons 36-43) of SEQ ID NO: 11, and the predicted amino acid sequence is shown in the corresponding encoded exons of SEQ ID NO:26 and the corresponding encoded exons of SEQ ID NO: 12, respectively.

In an embodiment the ZCCHC8-ROS1 nucleic acid molecule comprises sufficient ZCCHC8 and sufficient ROS1 sequence such that the encoded 5′ ZCCHC8-3′ ROS1 fusion has kinase activity, e.g., has elevated activity. In certain embodiments, the 5′ ZCCHC8-3′ ROS1 fusion comprises exons 1-2 from ZCCHC8 and exons 36-43 from ROS1. In certain embodiments, the ZCCHC8-ROS1 fusion comprises at least 1, 2 or more exons from ZCCHC8 and at least at least 1, 2, 3, 4, 5, 6, 7, 8 or more exons of ROS1. In certain embodiments, the ZCCHC8-ROS1 fusion comprises a fusion of exons 1-2 from ZCCHC8 and exon 36 from ROS1. In another embodiment, the ZCCHC8-ROS1 fusion comprises 1 or more exons of ZCCHC8; and at least 1, 2, 3, 4, 5, 6, 7, 8 or more exons of ROS1.

In one embodiment, the nucleic acid molecule includes a nucleotide sequence that has an in-frame fusion of intron 1 of ZCCHC8 (e.g., NM_{—}017612) with intron 35 of ROS1 (e.g., NM_{—}002944). In another embodiment, the nucleic acid molecule includes a nucleotide sequence that includes a breakpoint. For example, the nucleic acid molecule includes a nucleotide sequence that includes the fusion junction between the ZCCHC8 gene and the ROS1 gene, e.g., the breakpoint between intron 1 of ZCCHC8 and intron 35 of ROS1. In other embodiments, the nucleic acid molecules includes a nucleotide sequence of one or more of nucleotide of chromosome 12 coupled to (e.g., directly or indirectly juxtaposed to) one or more of nucleotide of chromosome 6. In one embodiment, the nucleic acid molecule includes the nucleotide sequence of: chromosome 12 at one or more of a nucleotide plus or minus 10, 20, 30, 40 50, 60, 80, 100, 150 nucleotides and chromosome 6 at one or more of a nucleotide plus or minus 10, 20, 30, 40 50, 60, 80, 100, 150 nucleotides (corresponding to the breakpoint of a ZCCHC8-ROS1 fusion), or a fragment thereof, or a sequence substantially identical thereto. In one embodiment, the nucleic acid molecule is complementary to at least a portion of a nucleotide sequence disclosed herein, e.g., is capable of hybridizing under a stringency condition described herein to SEQ ID NO:25 and/or SEQ ID NO: 11 or a fragment thereof. In yet other embodiment, the nucleic acid molecule hybridizes to a nucleotide sequence that is complementary to at least a portion of a nucleotide sequence disclosed herein, e.g., is capable of hybridizing under a stringency condition described herein to a nucleotide sequence complementary to SEQ ID NO:25 or SEQ ID NO: 11 or a fragment thereof.

In another embodiment, the ZCCHC8-ROS1 fusion nucleic acid comprises at least 6, 12, 15, 20, 25, 50, 75, 100 or more nucleotides from exons 1-2 of ZCCHC8 (e.g., from the nucleotide sequence of ZCCHC8 preceding the fusion junction with ROS1, e.g., of the ZCCHC8 sequence shown in SEQ ID NO:25), and at least 6, 12, 15, 20, 25, 50, 75, 100 or more nucleotides from exon 36 of ROS1 (e.g., from the nucleotide sequence of ROS1 following the fusion junction with ZCCHC8, e.g., of the ROS1 sequence shown in SEQ ID NO: 11)).

In other embodiments, the nucleic acid molecule includes a nucleotide sequence encoding a ZCCHC8-ROS1 fusion polypeptide that includes a fragment of a ZCCHC8 gene and a fragment of a ROS1 gene. In one embodiment, the nucleotide sequence encodes a ZCCHC8-ROS1 fusion polypeptide that includes e.g., a tyrosine kinase domain or a functional fragment thereof. In yet other embodiments, the nucleic acid molecule includes a nucleotide sequence encoding the amino acid sequence (e.g., a fragment of the amino acid sequence) shown in SEQ ID NO:26 and a nucleotide sequence encoding the amino acid sequence (e.g., a fragment of the amino acid sequence) shown in SEQ ID NO:12, or a fragment of the fusion, or a sequence substantially identical thereto. In one embodiment, the encoded ZCCHC8-ROS1 fusion polypeptide includes a tyrosine kinase domain or a functional fragment thereof.

In one embodiment, the ZCCHC8-ROS1 fusion includes the full sequence or a fragment of the nucleotide sequence shown in SEQ ID NO:23 and SEQ ID NO:96, or a nucleotide sequence substantially identical thereto. In another embodiment, the ZCCHC8-ROS1 fusion polypeptide includes the full sequence or a fragment of the amino acid sequence shown in SEQ ID NO:24 and SEQ ID NO:97, or an amino acid sequence substantially identical thereto, at least 70% or 80% or 905 or more identical.

In a related aspect, the invention features nucleic acid constructs that include the ZCCHC8-ROS1 nucleic acid molecules described herein. In certain embodiments, the nucleic acid molecules are operatively linked to a native or a heterologous regulatory sequence. Also included are vectors and host cells that include the ZCCHC8-ROS1 nucleic acid molecules described herein, e.g., vectors and host cells suitable for producing the nucleic acid molecules and polypeptides described herein.

In a related aspect, methods of producing the nucleic acid molecules and polypeptides described herein are also described.

In another aspect, the invention features nucleic acid molecules that reduce or inhibit the expression of a nucleic acid molecule that encodes a ZCCHC8-ROS1 fusion described herein. Examples of such nucleic acid molecules include, for example, antisense molecules, ribozymes, RNAi, triple helix molecules that hybridize to a nucleic acid encoding ZCCHC8-ROS1 or a transcription regulatory region of ZCCHC8-ROS1, and blocks or reduces mRNA expression of ZCCHC8-ROS1.

### Nucleic Acid Detection and Capturing Reagents

The invention also features a nucleic acid molecule, e.g., nucleic acid fragment, suitable as probe, primer, bait or library member that includes, flanks, hybridizes to, which are useful for identifying, or are otherwise based on, the ZCCHC8-ROS1 fusions described herein. In certain embodiments, the probe, primer or bait molecule is an oligonucleotide that allows capture, detection or isolation of a ZCCHC8-ROS1 fusion nucleic acid molecule described herein. The oligonucleotide can comprise a nucleotide sequence substantially complementary to a fragment of the ZCCHC8-ROS1 fusion nucleic acid molecules described herein. The sequence identity between the nucleic acid fragment, e.g., the oligonucleotide, and the target ZCCHC8-ROS1 sequence need not be exact, so long as the sequences are sufficiently complementary to allow the capture, detection or isolation of the target sequence. In one embodiment, the nucleic acid fragment is a probe or primer that includes an oligonucleotide between about 5 and 25, e.g., between 10 and 20, or 10 and 15 nucleotides in length. In other embodiments, the nucleic acid fragment is a bait that includes an oligonucleotide between about 100 to 300 nucleotides, 130 and 230 nucleotides, or 150 and 200 nucleotides, in length.

In one embodiment, the nucleic acid fragment can be used to identify or capture. e.g., by hybridization, a ZCCHC8-ROS1 fusion. For example, the nucleic acid fragment can be a probe, a primer, or a bait, for use in identifying or capturing, e.g., by hybridization, a ZCCHC8-ROS1 fusion described herein. In one embodiment, the nucleic acid fragment can be useful for identifying or capturing a ZCCHC8-ROS1 breakpoint, e.g., the nucleotide sequence of: chromosome 12 at nucleotide plus or minus 10, 20, 30, 40, 50, 60, 80, 100, 150 nucleotides and chromosome 6 at nucleotide plus or minus 10, 20, 30, 40 50, 60, 80, 100, 150 nucleotides.

In one embodiment, the nucleic acid fragment hybridizes to a nucleotide sequence within a chromosomal rearrangement that creates an in-frame fusion of intron 1 of ZCCHC8 with intron 35 of ROS1. In one embodiment, the nucleic acid fragment hybridizes to a nucleotide sequence in the region In other embodiments, the nucleic acid molecules includes a nucleotide sequence in the region of a nucleotide sequence of chromosome 12 coupled to (e.g., juxtaposed to) nucleotides in the region of a nucleotide sequence of chromosome 6. In one embodiment, the nucleic acid fragment hybridizes to a nucleotide sequence that includes a breakpoint, e.g., the nucleotide sequence of: chromosome 12 at nucleotide plus or minus 10, 20, 30, 40, 50, 60, 80, 100, 150 or more nucleotides and chromosome 6 at nucleotide plus or minus 10, 20, 30, 40, 50, 60, 80, 100, 150 or more nucleotides. For example, the nucleic acid fragment can hybridize to a nucleotide sequence that includes the fusion junction between the ZCCHC8 gene and the ROS1 gene, e.g., a nucleotide sequence that includes a portion of a nucleotide sequence within intron 1 of a ZCCHC8 gene and intron 35 of a ROS1 gene.

In another embodiment, the nucleic acid fragment hybridizes to a nucleotide sequence that comprises at least 6, 12, 15, 20, 25, 50, 75, 100, 150 or more nucleotides from exons 1-2 of ZCCHC8 (e.g., from the nucleotide sequence of ZCCHC8 preceding the fusion junction with ROS1, e.g., of the ZCCHC8 sequence shown in SEQ ID NO:25), and at least 6, 12, 15, 20, 25, 50, 75, 100, 150 or more nucleotides from exon 36 of ROS1 (e.g., from the nucleotide sequence of ROS1 following the fusion junction with ZCCHC8. e.g., of the ROS1 sequence shown in SEQ ID NO: 1).

The probes or primers described herein can be used, for example, for FISH detection or PCR amplification. In one exemplary embodiment where detection is based on PCR, amplification of the ZCCHC8-ROS1 fusion junction fusion junction can be performed using a primer or a primer pair, e.g., for amplifying a sequence flanking the fusion junctions described herein, e.g., the mutations or the junction of a chromosomal rearrangement described herein, e.g., ZCCHC8-ROS1.

In one embodiment, a pair of isolated oligonucleotide primers can amplify a region containing or adjacent to a position in the ZCCHC8-ROS1 fusion. For example, forward primers can be designed to hybridize to a nucleotide sequence within ZCCHC8 genomic or mRNA sequence (e.g., a nucleotide sequence within exons 1-2 of ZCCHC8 of SEQ ID NO:25), and the reverse primers can be designed to hybridize to a nucleotide sequence of ROS1 (e.g., a nucleotide sequence within exon 36 of ROS1, of SEQ ID NO: 11).

In another embodiment, the nucleic acid fragments can be used to identify, e.g., by hybridization, a ZCCHC8-ROS1 fusion. In one embodiment, the nucleic acid fragment hybridizes to a nucleotide sequence that includes a fusion junction between the ZCCHC8 transcript and the ROS1 transcript.

In other embodiments, the nucleic acid fragment includes a bait that comprises a nucleotide sequence that hybridizes to a ZCCHC8-ROS1 fusion nucleic acid molecule described herein, and thereby allows the capture or isolation said nucleic acid molecule. In one embodiment, a bait is suitable for solution phase hybridization. In other embodiments, a bait includes a binding entity, e.g., an affinity tag, that allows capture and separation, e.g., by binding to a binding entity, of a hybrid formed by a bait and a nucleic acid hybridized to the bait.

In other embodiments, the nucleic acid fragment includes a library member comprising a ZCCHC8-ROS1 nucleic acid molecule described herein. In one embodiment, the library member includes a rearrangement that results in a ZCCHC8-ROS1 fusion described herein.

The nucleic acid fragment can be detectably labeled with, e.g., a radiolabel, a fluorescent label, a bioluminescent label, a chemiluminescent label, an enzyme label, a binding pair label, or can include an affinity tag; a tag, or identifier (e.g., an adaptor, barcode or other sequence identifier).

### ZCCHC8-ROS1 Fusion Polypeptides

In another embodiment, the ZCCHC8-ROS1 fusion comprises an amino acid sequence (e.g., a fragment of the amino acid sequence) shown in SEQ ID NO:26 and an amino acid sequence (e.g., a fragment of the amino acid sequence) shown in SEQ ID NO:12, or a fragment of the fusion. In one embodiment, the ZCCHC8-ROS1 fusion comprises an amino acid sequence substantially identical to the amino acid sequence (e.g., a fragment of the amino acid sequence) shown in SEQ ID NO:26 and the amino acid sequence (e.g., a fragment of the amino acid sequence) shown in SEQ ID NO:12, or a fragment thereof. In one embodiment, the ZCCHC8-ROS1 fusion comprises an amino acid sequence at least 70%, at least 75%, at least 80%, at least 85%, at least 90%, at least 95%, at least 99%, at least 99.5 or greater, identical to the amino acid sequence (e.g., a fragment of the amino acid sequence) shown in SEQ ID NO:26 and the amino acid sequence (e.g., a fragment of the amino acid sequence) shown in SEQ ID NO:12. In one embodiment, the ZCCHC8-ROS1 fusion comprises a sequence containing at least 10, 20, 50, 100, 500, 600, 700, 800, 900, 1000, or more amino acids of the amino acid sequence shown in SEQ ID NO:26 and SEQ ID NO: 12. In one embodiment, the ZCCHC8-ROS1 fusion comprises an amino acid sequence containing at least 5, 10, 20, 50, 100, 500, 600, 700, 800, 900, 1000, or more contiguous amino acids of the amino acid sequence shown in SEQ ID NO:26 and at least 5, 10, 20, 50, 100, 500, 600, 700, 800, 900, 1000, or more contiguous amino acids of the amino acid sequence shown in SEQ ID NO:12. In one embodiment, the 5′ ZCCHC8-3′ ROS1 fusion polypeptide includes a receptor tyrosine kinase domain or a functional fragment thereof. In an embodiment, the 5′ZCCHC8-3′ROS1 fusion polypeptide comprises sufficient ROS1 and sufficient ZCCHC8 sequence such that it has kinase activity, e.g., has elevated activity.

In another aspect, the invention features a ZCCHC8-ROS1 fusion polypeptide (e.g., a purified ZCCHC8-ROS1 fusion polypeptide), a biologically active or antigenic fragment thereof, as well as reagents (e.g., antibody molecules that bind to a ZCCHC8-ROS1 fusion polypeptide), methods for modulating a ZCCHC8-ROS1 polypeptide activity and detection of a ZCCHC8-ROS1 polypeptide.

In one embodiment, the ZCCHC8-ROS1 fusion polypeptide has at least one biological activity. In one embodiment, at least one biological activity of the ZCCHC8-ROS1 fusion polypeptide is reduced or inhibited by an anti-cancer drug, e.g., a ZCCHC8 inhibitor, a ROS1 inhibitor. In one embodiment, at least one biological activity of the ZCCHC8-ROS1 fusion polypeptide is reduced or inhibited by a ROS1 inhibitor. In one embodiment, at least one biological activity of the ZCCHC8-ROS1 fusion polypeptide is reduced or inhibited by a ZCCHC8 inhibitor. In one embodiment, at least one biological activity of the ZCCHC8-ROS1 fusion polypeptide is reduced or inhibited by a ROS1 inhibitor, e.g., Ganetespib; Crizotinib; TAE684; a dual ALK and ROS1 inhibitor.

In yet other embodiments, the ZCCHC8-ROS1 fusion polypeptide is encoded by a nucleic acid molecule described herein. In one embodiment, the ZCCHC8-ROS1 fusion polypeptide is encoded by an in-frame fusion of intron 1 of ZCCHC8 with intron 35 of ROS1 (e.g., a sequence on chromosome 12 and a sequence on chromosome 6). In another embodiment, the ZCCHC8-ROS1 fusion polypeptide includes an amino acid sequence encoded by a nucleotide sequence comprising a fusion junction between the ZCCHC8 transcript and the ROS1 transcript.

In certain embodiments, the ZCCHC8-ROS1 fusion polypeptide comprises one or more of encoded exons 1-2 from ZCCHC8 and one or more of encoded exons 36-43 of ROS1. In certain embodiments, the ZCCHC8-ROS1 fusion polypeptide comprises at least 1, 2 or more encoded exons of ZCCHC8 and at least at least 1, 2, 3, 4, 5, 6, 7, 8 or more encoded exons of ROS1. In certain embodiments, the ZCCHC8-ROS1 fusion polypeptide comprises a fusion of encoded exons 1-2 from ZCCHC8 and encoded exon 36 from ROS1 (or a fragment thereof). In other embodiments, the fusion comprises at least 1 or more encoded exons of ZCCHC8; and at least 1, 2, 3, 4, 5, 6, 7, 8 or more encoded exons of ROS1. In certain embodiments, the ZCCHC8-ROS1 fusion polypeptide comprises encoded exons 1-2 from ZCCHC8 and exons 36-43 of ROS1. In certain embodiments, the 5′ ZCCHC8-3′ ROS1 fusion polypeptide comprises a fusion junction of the sequence of exons 1-2 from ZCCHC8 and the sequence of exon 36 from ROS1.

In certain embodiments, the ZCCHC8-ROS1 fusion comprises the amino acid sequence corresponding to exons 1-2 or a fragment thereof from ZCCHC8, and the amino acid sequence corresponding to exon 36 or a fragment thereof from ROS1 (e.g., as shown in SEQ ID NO:26 and SEQ ID NO:12). In one embodiment, the ZCCHC8-ROS1 fusion comprises at least 5, 10, 15, 20 or more amino acids from exons 1-2 of ZCCHC8 (e.g., from the amino acid sequence of ZCCHC8 preceding the fusion junction with ROS1, e.g., of the ZCCHC8 sequence shown in SEQ ID NO:26), and at least 5, 10, 15, 20 or more amino acids from exon 36 of ROS1 (e.g., from the amino acid sequence of ROS1 following the fusion junction with ZCCHC8, e.g., of the ROS1 sequence shown in SEQ ID NO: 12).

In one embodiment, the ZCCHC8-ROS1 fusion polypeptide includes a tyrosine kinase domain or a functional fragment thereof. In a related aspect, the invention features ZCCHC8-ROS1 fusion polypeptide or fragments operatively linked to heterologous polypeptides to form fusion proteins.

In another embodiment, the ZCCHC8-ROS1 fusion polypeptide or fragment is a peptide, e.g., an immunogenic peptide or protein. This peptide or protein contains a fusion junction described herein. Such immunogenic peptides or proteins can be used to raise antibodies specific to the fusion protein. In other embodiments, such immunogenic peptides or proteins can be used for vaccine preparation. The vaccine preparation can include other components, e.g., an adjuvant.

In another aspect, the invention features antibody molecules that bind to a ZCCHC8-ROS1 fusion polypeptide or fragment described herein. In embodiments, the antibody can distinguish wild type ROS1 (or ZCCHC8) from ZCCHC8-ROS1.

### Detection Reagents and Detection of Mutations

In another aspect, the invention features a detection reagent, e.g., a purified or an isolated preparation thereof. Detection reagents can distinguish a nucleic acid, or protein sequence, having a breakpoint, e.g., a ZCCHC8-ROS1 breakpoint; from a reference sequence. In one embodiment, the detection reagent detects (e.g., specifically detects) a ZCCHC8-ROS1 fusion nucleic acid or a polypeptide, e.g., distinguishes a wild type ROS1 or another ROS1 fusion (or ZCCHC8) from a ZCCHC8-ROS1 nucleic acid (e.g., as described herein in SEQ ID NO:25 and SEQ ID NO: 11); or a ZCCHC8-ROS1 polypeptide (e.g., as described herein in SEQ ID NO:26 and SEQ ID NO: 12).

Detection reagents, e.g., nucleic acid-based detection reagents, can be used to identify mutations in a target nucleic acid, e.g., DNA, e.g., genomic DNA or cDNA, or RNA, e.g., in a sample, e.g., a sample of nucleic acid derived from a neoplastic or tumor cell, e.g., a melanocytic neoplasm, melanoma or metastatic cell. Detection reagents, e.g., antibody-based detection reagents, can be used to identify mutations in a target protein, e.g., in a sample, e.g., a sample of protein derived from, or produced by, a neoplastic or tumor cell, e.g., a melanocytic neoplasm, melanoma or metastatic cell.

### Method of Treatment

In a related aspect, a method of inhibiting, reducing, or treating a hyperproliferative disorder, e.g., a neoplasm (including benign, pre-malignant or malignant (e.g., a cancer), in a subject is provided. The method includes administering to the subject a preselected therapeutic agent. e.g., an anti-cancer agent (e.g., a kinase inhibitor), as a single agent, or in combination, in an amount sufficient to reduce, inhibit or treat the activity or expression of ZCCHC8-ROS1 (e.g., a ZCCHC8-ROS1 fusion described herein), thereby inhibiting, reducing, or treating the hyperproliferative disorder in the subject.

In one embodiment, the subject treated has a ZCCHC8-ROS1 fusion; e.g., the subject has a tumor or cancer harboring a ZCCHC8-ROS1 fusion. In other embodiments, the subject has been previously identified as having a ZCCHC8-ROS1 fusion. In yet other embodiments, the subject has been previously identified as being likely or unlikely to respond to treatment with a protein kinase inhibitor, e.g., a subject that has previously participated in a clinical trial. In other embodiments, the subject has been previously identified as being likely or unlikely to respond to treatment with a protein kinase inhibitor, based on the presence of the ZCCHC8-ROS1 fusion. In one embodiment, the subject is a mammal, e.g., a human. In one embodiment, the subject has, or at risk of having a cancer at any stage of disease. In other embodiments, the subject is a patient, e.g., a cancer patient, particularly a melanoma patient having such a fusion.

In certain embodiments, the neoplasm or neoplastic cell is a benign, pre-malignant, malignant (cancer) or metastasis. In certain embodiments, the cancer is a solid tumor, a soft tissue tumor, or a metastatic lesion.

In one embodiment, the neoplasm or neoplastic cell is a benign, pre-malignant, malignant (cancer) or metastasis. In certain embodiments, the cancer is a solid tumor, a soft tissue tumor, or a metastatic lesion. In one embodiment, the cancer is a carcinoma. In one embodiment, the cancer is an adenocarcinoma. In an embodiment, the cancer is a lung adenocarcinoma. In one embodiment, the cancer is chosen from a lung cancer, a pancreatic cancer, melanoma, a colorectal cancer, an esophageal-gastric cancer, a thyroid cancer, or an adenocarcinoma. In one embodiment, the cancer is a lung adenocarcinoma. In other embodiment, the lung cancer is chosen from one or more of the following: non-small cell lung cancer (NSCLC), small cell lung cancer (SCLC), squamous cell carcinoma (SCC), adenocarcinoma of the lung, bronchogenic carcinoma, or a combination thereof. In one embodiment, the lung cancer is NSCLC or SCC.

In one embodiment, the anti-cancer agent is a ROS1 inhibitor. In one embodiment, the anti-cancer agent is a ZCCHC8 inhibitor. In one embodiment, the anti-cancer agent is a kinase inhibitor. In one embodiment, the anti-cancer agent is a ROS1 inhibitor, e.g., Ganetespib; Crizotinib; TAE684; AP26113, X-276, X-376, X-396, CH5424802 (AF-802), GSK1838705, ASP3026, PHA-E429, CRL151104; and additional examples of kinase inhibitors are described in de la Bellacasa R. P. et al, Transl Lung Cancer Res 2013; 2(2):72-86.

### MYO5A-ROS1 Fusions

In one embodiment, a fusion includes an in-frame fusion of an exon of myosin VA (heavy chain 12 myoxin) (MYO5A), e.g., one more exons of MYO5A (e.g., one or more of exons 1-23 of MYO5A) or a fragment thereof, and an exon of C-Ros oncogene 1 (ROS1), e.g., one or more exons of a ROS1 (e.g., one or more of exons 35-43 of ROS1) or a fragment thereof. For example, the MYO5A-ROS1 fusion can include an in-frame fusion within an intron of MYO5A (e.g., intron 23) or a fragment thereof, with an intron of ROS1 (e.g., intron 34) or a fragment thereof. In one embodiment, the fusion of the MYO5A-ROS1 fusion comprises the nucleotide sequence of: chromosome 15 at one or more of a nucleotide (plus or minus 10, 20, 30, 50, 60, 70, 80, 100 or more nucleotides) and chromosome 6 at one or more of a nucleotide (plus or minus 10, 20, 30, 50, 60, 70, 80, 100 or more nucleotides). In one embodiment, the MYO5A-ROS1 fusion is a translocation, e.g., a translocation of a portion of chromosome 15 and a portion of chromosome 6.

In certain embodiments, the MYO5A-ROS1 fusion is in a 5′-MYO5A to 3′-ROS1 configuration (also referred to herein as “5′-MYO5A-ROS1-3′).” The term “fusion” or “fusion molecule” can refer to a polypeptide or a nucleic acid fusion, depending on the context. It may include a full-length sequence of a fusion or a fragment thereof, e.g., a fusion junction (e.g., a fragment including a portion of MYO5A and a portion of ROS1. e.g., a portion of the MYO5A-ROS1 fusion described herein). In one embodiment, the MYO5A-ROS1 fusion polypeptide includes a fragment of the amino acid sequence shown in SEQ ID NO:30 and a fragment of the amino acid sequence shown in SEQ ID NO: 12, or an amino acid sequence substantially identical thereto. In another embodiment, the MYO5A-ROS1 fusion nucleic acid includes a fragment of the nucleotide sequence shown in SEQ ID NO:29 and a fragment of the nucleotide sequence shown in SEQ ID NO: 11, or a nucleotide sequence substantially identical thereto. In one embodiment, the MYO5A-ROS1 fusion polypeptide comprises sufficient MYO5A and sufficient ROS1 sequence such that the 5′ MYO5A-3′ ROS1 fusion has kinase activity, e.g., has elevated activity, e.g., tyrosine kinase activity. In any event, the fusion causes activation in the cells harboring it of oncogenic signaling pathways

In certain embodiments, the MYO5A-ROS1 fusion comprises one or more (or all of) exons 1-23 from MYO5A and one or more (or all of) exons 35-43 of ROS1 (e.g., one or more of the exons shown in SEQ ID NO:29 and SEQ ID NO: 11. In another embodiment, the MYO5A-ROS1 fusion comprises one or more (or all of) exons 1-23 of MYO5A and one or more (or all of) exons 35-43 of ROS1. In certain embodiments, the MYO5A-ROS1 fusion comprises at least 1, 2, 3, 4, 5, 6, 7, 8, 9, 10, 11, 12, 13, 14, 15, 16, 17, 18, 19, 20, 21, 22, 23 or more exons (or encoded exons) from MYO5A and at least 1, 2, 3, 4, 5, 6, 7, 8, 9 or more exons (or encoded exons) from ROS1 (e.g., from the MYO5A and ROS1 sequences shown in SEQ ID NO:29 and SEQ ID NO:30 and SEQ ID NO: 11 and SEQ ID NO:12.

In certain embodiments, the MYO5A-ROS1 fusion comprises exons 1-23 or a fragment thereof from MYO5A, and exons 35-43 or a fragment thereof from ROS1 (e.g., as shown in SEQ ID NO:29 and SEQ ID NO:11). In one embodiment, the MYO5A-ROS1 fusion comprises at least 5, 10, 15, 20, 30, 40, 50 or more amino acids from exons 1-23 of MYO5A (e.g., from the amino acid sequence of MYO5A as shown in SEQ ID NO:30 (e.g., from the amino acid sequence of MYO5A preceding the fusion junction with ROS1, and at least 5, 10, 15, 20, 30, 40, 50 or more amino acids from exons 35-43 of ROS1 (e.g., from the amino acid sequence of ROS1 as shown in SEQ ID NO: 12). In another embodiment, the MYO5A-ROS1 fusion comprises at least 6, 12, 15, 20, 25, 50, 75, 100 or more nucleotides from exons 1-23 of MYO5A (e.g., from the nucleotide sequence of MYO5A as shown in 125 (SEQ ID NO:29) (e.g., from the nucleotide sequence of MYO5A preceding the fusion junction with ROS1); and at least 6, 12, 15, 20, 25, 50, 75, 100 or more nucleotides from exons 35-43 of ROS1 (e.g., from the nucleotide sequence of ROS1 as shown in 112 (SEQ ID NO: 11)).

### MYO5A-ROS1 Nucleic Acid Molecules

In one aspect, the invention features a nucleic acid molecule (e.g., an isolated or purified) nucleic acid molecule that includes a fragment of a MYO5A gene and a fragment of a ROS1 gene. In one embodiment, the nucleotide sequence encodes a MYO5A-ROS1 fusion polypeptide that includes a tyrosine kinase domain or a functional fragment thereof. In another embodiment, the nucleotide sequence encodes a fragment of the ROS1 polypeptide including the amino acid sequence of SEQ ID NO: 12 or a fragment thereof, or a sequence substantially identical thereto. In other embodiments, the nucleic acid molecule includes a fragment of the MYO5A gene encoding the amino acid sequence of SEQ ID NO:30 or a fragment thereof, or a sequence substantially identical thereto. In yet other embodiments, the nucleic acid molecule includes a nucleotide sequence encoding the amino acid sequence shown in 126 SEQ ID NO:30, or a fragment thereof, and the amino acid sequence shown in 113 SEQ ID NO: 12 or a fragment thereof, or a sequence substantially identical thereto.

In one embodiment, the nucleic acid molecule includes a fusion, e.g., an in-frame fusion, between an intron of MYO5A (e.g., intron 23, or a fragment thereof), and an intron of ROS1 (e.g., intron 34, or a fragment thereof). The MYO5A-ROS1 fusion can comprise a fusion of the nucleotide sequence of: chromosome 15 at one or more of a nucleotide (plus or minus 10, 20, 30, 50, 60, 70, 80, 100 nucleotides) and chromosome 6 at one or more of a nucleotide (plus or minus 10, 20, 30, 50, 60, 70, 80, 100 nucleotides), or a fragment thereof. In one embodiment, the MYO5A-ROS1 fusion comprises a fusion of the nucleotide sequence of: chromosome 15 at one or more of a nucleotide (plus or minus 10, 20, 30, 50, 60, 70, 80, 100 nucleotides) and chromosome 6 at one or more of a nucleotide (plus or minus 10, 20, 30, 50, 60, 70, 80, 100 nucleotides), or a fragment thereof.

In another embodiment, the MYO5A-ROS1 fusion comprises a nucleotide sequence (e.g., a fragment of a nucleotide sequence) shown in 125 SEQ ID NO:29 and a nucleotide sequence (e.g., a fragment of a nucleotide sequence) shown in SEQ ID NO: 11, or a fragment of the fusion. In one embodiment, the MYO5A-ROS1 fusion comprises a nucleotide sequence substantially identical to the nucleotide sequence (e.g., a fragment of a nucleotide sequence) shown in SEQ ID NO:29 and the nucleotide sequence (e.g., a fragment of a nucleotide sequence) shown SEQ ID NO: 11, or a fragment of the fusion. In one embodiment, the MYO5A-ROS1 fusion comprises a nucleotide sequence at least 70%, at least 75%, at least 80%, at least 85%, at least 90%, at least 95%, at least 99%, at least 99.5 or greater, identical to the nucleotide sequence (e.g., a fragment of a nucleotide sequence) shown in SEQ ID NO:29 and to the nucleotide sequence (e.g., a fragment of a nucleotide sequence) shown in SEQ ID NO: 11. In one embodiment, the MYO5A-ROS1 fusion comprises a nucleotide sequence containing at least 25, 50, 100, 150, 200, 500, 1000, 1500, 2000, 2500, 3000, or more nucleotides of the nucleotide sequence shown in SEQ ID NO:29 and a nucleotide sequence containing at least 25, 50, 100, 150, 200, 500, 1000, 1500, 2000, 2500, 3000, or more nucleotides of the nucleotide sequence shown in SEQ ID NO: 11. In one embodiment, the MYO5A-ROS1 fusion comprises a nucleotide sequence containing at least 25, 50, 100, 150, 200, 500, 1000, 1500, 2000, 2500, 3000, or more contiguous nucleotides of the nucleotide sequence shown in SEQ ID NO:29 and a nucleotide sequence containing at least 25, 50, 100, 150, 200, 500, 1000, 1500, 2000, 2500, 3000, or more nucleotides of the nucleotide sequence shown in SEQ ID NO:11.

In another embodiment, the nucleic acid molecule includes a fusion, e.g., an in-frame fusion, of at least 1, 2, 3, 4, 5, 6, 7, 8, 9, 10, 11, 12, 13, 14, 15, 16, 17, 18, 19, 20, 21, 22, 23 or more exons of MYO5A or a fragment thereof (e.g., one or more of exons 1-23 of MYO5A or a fragment thereof), and at least 1, 2, 3, 4, 5, 6, 7, 8, 9 or more exons of ROS1 or a fragment thereof (e.g., one or more of exons 35-43 of ROS1 or a fragment thereof). In yet other embodiments, the nucleic acid molecule includes a fragment the nucleotide sequence shown in SEQ ID NO:29 and a fragment of the nucleotide sequence shown in SEQ ID NO:11 or a fragment of the fusion, or a sequence substantially identical thereto.

In one embodiment, the nucleic acid molecule is complementary to at least a portion of a nucleotide sequence disclosed herein, e.g., is capable of hybridizing under a stringency condition described herein to SEQ ID NO:29 and/or SEQ ID NO: 11, or a fragment thereof. In yet another embodiment, the nucleic acid molecule hybridizes to a nucleotide sequence that is complementary to at least a portion of a nucleotide sequence disclosed herein, e.g., is capable of hybridizing under a stringency condition to a nucleotide sequence complementary to SEQ ID NO:29 and/or SEQ ID NO: 11, or a fragment thereof. The nucleotide sequence of a cDNA encoding an exemplary 5′ MYO5A-3′ ROS1 fusion is shown in at least exon 23 (e.g., exons 1-23) of SEQ ID NO:29 and at least exon 35 (e.g., exons 35-43) of SEQ ID NO: 11, and the predicted amino acid sequence is shown in the corresponding encoded exons of SEQ ID NO:30 and the corresponding encoded exons of SEQ ID NO: 12, respectively.

In an embodiment the MYO5A-ROS1 nucleic acid molecule comprises sufficient MYO5A and sufficient ROS1 sequence such that the encoded 5′ MYO5A-3′ ROS1 fusion has kinase activity, e.g., has elevated activity. In certain embodiments, the 5′ MYO5A-3′ ROS1 fusion comprises exons 1-23 from MYO5A and exons 35-43 from ROS1. In certain embodiments, the MYO5A-ROS1 fusion comprises at least 1, 2, 3, 4, 5, 6, 7, 8, 9, 10, 11, 12, 13, 14, 15, 16, 17, 18, 19, 20, 21, 22, 23 or more exons from MYO5A and at least at least 1, 2, 3, 4, 5, 6, 7, 8, or more exons of ROS1. In certain embodiments, the MYO5A-ROS1 fusion comprises a fusion of exon 23 from MYO5A and exon 35 from ROS1. In another embodiment, the MYO5A-ROS1 fusion comprises 1, 2, 3, 4, 5, 6, 7, 8, 9, 10, 1, 12, 13, 14, 15, 16, 17, 18, 19, 20, 21, 22, 23 or more exons of MYO5A; and at least 1, 2, 3, 4, 5, 6, 7, 8, 9 or more exons of ROS1.

In one embodiment, the nucleic acid molecule includes a nucleotide sequence that has an in-frame fusion of intron 23 of MYO5A (e.g., NM_{—}000259) with intron 34 of ROS1 (e.g., NM_{—}002944). In another embodiment, the nucleic acid molecule includes a nucleotide sequence that includes a breakpoint. For example, the nucleic acid molecule includes a nucleotide sequence that includes the fusion junction between the MYO5A gene and the ROS1 gene, e.g., the breakpoint between intron 23 of MYO5A and intron 34 of ROS1. In other embodiments, the nucleic acid molecules includes a nucleotide sequence of one or more of nucleotide of chromosome 15 coupled to (e.g., directly or indirectly juxtaposed to) one or more of nucleotide of chromosome 6. In one embodiment, the nucleic acid molecule includes the nucleotide sequence of: chromosome 15 at one or more of a nucleotide plus or minus 10, 20, 30, 40 50, 60, 80, 100, 150 nucleotides and chromosome 6 at one or more of a nucleotide plus or minus 10, 20, 30, 40, 50, 60, 80, 100, 150 nucleotides (corresponding to the breakpoint of a MYO5A-ROS1 fusion), or a fragment thereof, or a sequence substantially identical thereto. In one embodiment, the nucleic acid molecule is complementary to at least a portion of a nucleotide sequence disclosed herein, e.g., is capable of hybridizing under a stringency condition described herein to SEQ ID NO:29 and/or SEQ ID NO:11 or a fragment thereof. In yet other embodiment, the nucleic acid molecule hybridizes to a nucleotide sequence that is complementary to at least a portion of a nucleotide sequence disclosed herein, e.g., is capable of hybridizing under a stringency condition described herein to a nucleotide sequence complementary to SEQ ID NO:29 or SEQ ID NO: 1 or a fragment thereof.

In another embodiment, the MYO5A-ROS1 fusion nucleic acid comprises at least 6, 12, 15, 20, 25, 50, 75, 100 or more nucleotides from exon 23 of MYO5A (e.g., from the nucleotide sequence of MYO5A preceding the fusion junction with ROS1, e.g., of the MYO5A sequence shown in SEQ ID NO:29), and at least 6, 12, 15, 20, 25, 50, 75, 100 or more nucleotides from exon 35 of ROS1 (e.g., from the nucleotide sequence of ROS1 following the fusion junction with MYO5A, e.g., of the ROS1 sequence shown in SEQ ID NO: 1).

In other embodiments, the nucleic acid molecule includes a nucleotide sequence encoding a MYO5A-ROS1 fusion polypeptide that includes a fragment of a MYO5A gene and a fragment of a ROS1 gene. In one embodiment, the nucleotide sequence encodes a MYO5A-ROS1 fusion polypeptide that includes e.g., a tyrosine kinase domain or a functional fragment thereof. In yet other embodiments, the nucleic acid molecule includes a nucleotide sequence encoding the amino acid sequence (e.g., a fragment of the amino acid sequence) shown in SEQ ID NO:30 and a nucleotide sequence encoding the amino acid sequence (e.g., a fragment of the amino acid sequence) shown in SEQ ID NO:12, or a fragment of the fusion, or a sequence substantially identical thereto. In one embodiment, the encoded MYO5A-ROS1 fusion polypeptide includes a tyrosine kinase domain or a functional fragment thereof.

In one embodiment, the MYO5A-ROS1 fusion includes the full sequence or a fragment of the nucleotide sequence shown in SEQ ID NO:27 and SEQ ID NO:84, or a nucleotide sequence substantially identical thereto. In another embodiment, the MYO5A-ROS1 fusion polypeptide includes the full sequence or a fragment of the amino acid sequence shown in SEQ ID NO:28 and SEQ ID NO:85, or an amino acid sequence substantially identical thereto, for example at least 70%, 80% 90% identical or even more.

In a related aspect, the invention features nucleic acid constructs that include the MYO5A-ROS1 nucleic acid molecules described herein. In certain embodiments, the nucleic acid molecules are operatively linked to a native or a heterologous regulatory sequence. Also included are vectors and host cells that include the MYO5A-ROS1 nucleic acid molecules described herein, e.g., vectors and host cells suitable for producing the nucleic acid molecules and polypeptides described herein.

In a related aspect, methods of producing the nucleic acid molecules and polypeptides described herein are also described.

In another aspect, the invention features nucleic acid molecules that reduce or inhibit the expression of a nucleic acid molecule that encodes a MYO5A-ROS1 fusion described herein. Examples of such nucleic acid molecules include, for example, antisense molecules, ribozymes, RNAi, triple helix molecules that hybridize to a nucleic acid encoding MYO5A-ROS1, or a transcription regulatory region of MYO5A-ROS1, and blocks or reduces mRNA expression of MYO5A-ROS1.

### Nucleic Acid Detection and Capturing Reagents

The invention also features a nucleic acid molecule, e.g., nucleic acid fragment, suitable as probe, primer, bait or library member that includes, flanks, hybridizes to, which are useful for identifying, or are otherwise based on, the MYO5A-ROS1 fusions described herein. In certain embodiments, the probe, primer or bait molecule is an oligonucleotide that allows capture, detection or isolation of a MYO5A-ROS1 fusion nucleic acid molecule described herein. The oligonucleotide can comprise a nucleotide sequence substantially complementary to a fragment of the MYO5A-ROS1 fusion nucleic acid molecules described herein. The sequence identity between the nucleic acid fragment, e.g., the oligonucleotide, and the target MYO5A-ROS1 sequence need not be exact, so long as the sequences are sufficiently complementary to allow the capture, detection or isolation of the target sequence. In one embodiment, the nucleic acid fragment is a probe or primer that includes an oligonucleotide between about 5 and 25, e.g., between 10 and 20, or 10 and 15 nucleotides in length. In other embodiments, the nucleic acid fragment is a bait that includes an oligonucleotide between about 100 to 300 nucleotides, 130 and 230 nucleotides, or 150 and 200 nucleotides, in length.

In one embodiment, the nucleic acid fragment can be used to identify or capture, e.g., by hybridization, a MYO5A-ROS1 fusion. For example, the nucleic acid fragment can be a probe, a primer, or a bait, for use in identifying or capturing, e.g., by hybridization, a MYO5A-ROS1 fusion described herein. In one embodiment, the nucleic acid fragment can be useful for identifying or capturing a MYO5A-ROS1 breakpoint, e.g., the nucleotide sequence of: chromosome 15 at nucleotide plus or minus 10, 20, 30, 40, 50, 60, 80, 100, 150 nucleotides and chromosome 6 at nucleotide plus or minus 10, 20, 30, 40 50, 60, 80, 100, 150 nucleotides.

In one embodiment, the nucleic acid fragment hybridizes to a nucleotide sequence within a chromosomal rearrangement that creates an in-frame fusion of intron 23 of MYO5A with intron 34 of ROS1. In one embodiment, the nucleic acid fragment hybridizes to a nucleotide sequence in the region In other embodiments, the nucleic acid molecules includes a nucleotide sequence in the region of a nucleotide sequence of chromosome 15 coupled to (e.g., juxtaposed to) nucleotides in the region of a nucleotide sequence Y of chromosome 6. In one embodiment, the nucleic acid fragment hybridizes to a nucleotide sequence that includes a breakpoint, e.g., the nucleotide sequence of: chromosome 15 at nucleotide plus or minus 10, 20, 30, 40 50, 60, 80, 100, 150 or more nucleotides and chromosome 6 at nucleotide plus or minus 10, 20, 30, 40 50, 60, 80, 100, 150 or more nucleotides. For example, the nucleic acid fragment can hybridize to a nucleotide sequence that includes the fusion junction between the MYO5A gene and the ROS1 gene, e.g., a nucleotide sequence that includes a portion of a nucleotide sequence within intron 23 of a MYO5A gene and intron 34 of a ROS1 gene.

In another embodiment, the nucleic acid fragment hybridizes to a nucleotide sequence that comprises at least 6, 12, 15, 20, 25, 50, 75, 100, 150 or more nucleotides from exon 23 of MYO5A (e.g., from the nucleotide sequence of MYO5A preceding the fusion junction with ROS1, e.g., of the MYO5A sequence shown in SEQ ID NO:29), and at least 6, 12, 15, 20, 25, 50, 75, 100, 150 or more nucleotides from exon 35 of ROS1 (e.g., from the nucleotide sequence of ROS1 following the fusion junction with MYO5A, e.g., of the ROS1 sequence shown in SEQ ID NO: 11).

The probes or primers described herein can be used, for example, for FISH detection or PCR amplification. In one exemplary embodiment where detection is based on PCR, amplification of the MYO5A-ROS1 fusion junction fusion junction can be performed using a primer or a primer pair, e.g., for amplifying a sequence flanking the fusion junctions described herein, e.g., the mutations or the junction of a chromosomal rearrangement described herein, e.g., MYO5A-ROS1.

In one embodiment, a pair of isolated oligonucleotide primers can amplify a region containing or adjacent to a position in the MYO5A-ROS1 fusion. For example, forward primers can be designed to hybridize to a nucleotide sequence within MYO5A genomic or mRNA sequence (e.g., a nucleotide sequence within exon 23 of MYO5A of SEQ ID NO:29), and the reverse primers can be designed to hybridize to a nucleotide sequence of ROS1 (e.g., a nucleotide sequence within exon 35 of ROS1, of SEQ ID NO:11).

In another embodiment, the nucleic acid fragments can be used to identify, e.g., by hybridization, a MYO5A-ROS1 fusion. In one embodiment, the nucleic acid fragment hybridizes to a nucleotide sequence that includes a fusion junction between the MYO5A transcript and the ROS1 transcript.

In other embodiments, the nucleic acid fragment includes a bait that comprises a nucleotide sequence that hybridizes to a MYO5A-ROS1 fusion nucleic acid molecule described herein, and thereby allows the capture or isolation said nucleic acid molecule. In one embodiment, a bait is suitable for solution phase hybridization. In other embodiments, a bait includes a binding entity, e.g., an affinity tag, that allows capture and separation, e.g., by binding to a binding entity, of a hybrid formed by a bait and a nucleic acid hybridized to the bait.

In other embodiments, the nucleic acid fragment includes a library member comprising a MYO5A-ROS1 nucleic acid molecule described herein. In one embodiment, the library member includes a rearrangement that results in a MYO5A-ROS1 fusion described herein.

The nucleic acid fragment can be detectably labeled with, e.g., a radiolabel, a fluorescent label, a bioluminescent label, a chemiluminescent label, an enzyme label, a binding pair label, or can include an affinity tag; a tag, or identifier (e.g., an adaptor, barcode or other sequence identifier).

### MYO5A-ROS1 Fusion Polypeptides

In another embodiment, the MYO5A-ROS1 fusion comprises an amino acid sequence (e.g., a fragment of the amino acid sequence) shown in SEQ ID NO:30 and an amino acid sequence (e.g., a fragment of the amino acid sequence) shown in SEQ ID NO:12, or a fragment of the fusion. In one embodiment, the MYO5A-ROS1 fusion comprises an amino acid sequence substantially identical to the amino acid sequence (e.g., a fragment of the amino acid sequence) shown in SEQ ID NO:30 and the amino acid sequence (e.g., a fragment of the amino acid sequence) shown in SEQ ID NO:12, or a fragment thereof. In one embodiment, the MYO5A-ROS1 fusion comprises an amino acid sequence at least 70%, at least 75%, at least 80%, at least 85%, at least 90%, at least 95%, at least 99%, at least 99.5 or greater, identical to the amino acid sequence (e.g., a fragment of the amino acid sequence) shown in SEQ ID NO:30 and the amino acid sequence (e.g., a fragment of the amino acid sequence) shown in SEQ ID NO:12. In one embodiment, the MYO5A-ROS1 fusion comprises a sequence containing at least 10, 20, 50, 100, 500, 600, 700, 800, 900, 1000, or more amino acids of the amino acid sequence shown in SEQ ID NO:30 and SEQ ID NO: 12. In one embodiment, the MYO5A-ROS1 fusion comprises an amino acid sequence containing at least 5, 10, 20, 50, 100, 500, 600, 700, 800, 900, 1000, or more contiguous amino acids of the amino acid sequence shown in SEQ ID NO:30 and at least 5, 10, 20, 50, 100, 500, 600, 700, 800, 900, 1000, or more contiguous amino acids of the amino acid sequence shown in SEQ ID NO: 12. In one embodiment, the 5′ MYO5A-3′ ROS1 fusion polypeptide includes a receptor tyrosine kinase domain or a functional fragment thereof. In an embodiment, the 5′MYO5A-3′ROS1 fusion polypeptide comprises sufficient ROS1 and sufficient MYO5A sequence such that it has kinase activity, e.g., has elevated activity. In any event, the fusion causes activation in the cells harboring it of oncogenic signaling pathways.

In another aspect, the invention features a MYO5A-ROS1 fusion polypeptide (e.g., a purified MYO5A-ROS1 fusion polypeptide), a biologically active or antigenic fragment thereof, as well as reagents (e.g., antibody molecules that bind to a MYO5A-ROS1 fusion polypeptide), methods for modulating a MYO5A-ROS1 polypeptide activity and detection of a MYO5A-ROS1 polypeptide.

In one embodiment, the MYO5A-ROS1 fusion polypeptide has at least one biological activity. In one embodiment, at least one biological activity of the MYO5A-ROS1 fusion polypeptide is reduced or inhibited by an anti-cancer drug, e.g., a MYO5A inhibitor, a ROS1 inhibitor. In one embodiment, at least one biological activity of the MYO5A-ROS1 fusion polypeptide is reduced or inhibited by a ROS1 inhibitor. In one embodiment, at least one biological activity of the MYO5A-ROS1 fusion polypeptide is reduced or inhibited by a MYO5A inhibitor. In one embodiment, at least one biological activity of the MYO5A-ROS1 fusion polypeptide is reduced or inhibited by a ROS1 inhibitor, e.g., Ganetespib; Crizotinib; TAE684; a dual ALK and ROS1 inhibitor.

In yet other embodiments, the MYO5A-ROS1 fusion polypeptide is encoded by a nucleic acid molecule described herein. In one embodiment, the MYO5A-ROS1 fusion polypeptide is encoded by an in-frame fusion of intron 23 of MYO5A with intron 34 of ROS1 (e.g., a sequence on chromosome 15 and a sequence on chromosome 6). In another embodiment, the MYO5A-ROS1 fusion polypeptide includes an amino acid sequence encoded by a nucleotide sequence comprising a fusion junction between the MYO5A transcript and the ROS1 transcript.

In certain embodiments, the MYO5A-ROS1 fusion polypeptide comprises one or more of encoded exons 1-23 from MYO5A and one or more of encoded exons 35-43 of ROS1. In certain embodiments, the MYO5A-ROS1 fusion polypeptide comprises at least 1, 2, 3, 4, 5, 6, 7, 8, 9, 10, 11, 12, 13, 14, 15, 16, 17, 18, 19, 20, 21, 22, 23 or more encoded exons of MYO5A and at least at least 1, 2, 3, 4, 5, 6, 7, 8, 9 or more encoded exons of ROS1. In certain embodiments, the MYO5A-ROS1 fusion polypeptide comprises a fusion of encoded exon 23 from MYO5A and encoded exon 35 from ROS1 (or a fragment thereof). In other embodiments, the fusion comprises at least 1, 2, 3, 4, 5, 6, 7, 8, 9, 10, 11, 12, 13, 14, 15, 16, 17, 18, 19, 20, 21, 22, 23 or more encoded exons of MYO5A; and at least 1, 2, 3, 4, 5, 6, 7, 8, 9 or more encoded exons of ROS1. In certain embodiments, the MYO5A-ROS1 fusion polypeptide comprises encoded exons 1-23 from MYO5A and exons 35-43 of ROS1. In certain embodiments, the 5′ MYO5A-3′ ROS1 fusion polypeptide comprises a fusion junction of the sequence of exon 23 from MYO5A and the sequence of exon 35 from ROS1.

In certain embodiments, the MYO5A-ROS1 fusion comprises the amino acid sequence corresponding to exon 23 or a fragment thereof from MYO5A, and the amino acid sequence corresponding to exon 35 or a fragment thereof from ROS1 (e.g., as shown in SEQ ID NO:30 and SEQ ID NO:12). In one embodiment, the MYO5A-ROS1 fusion comprises at least 5, 10, 15, 20 or more amino acids from exon 23 of MYO5A (e.g., from the amino acid sequence of MYO5A preceding the fusion junction with ROS1, e.g., of the MYO5A sequence shown in SEQ ID NO:30), and at least 5, 10, 15, 20 or more amino acids from exon 35 of ROS1 (e.g., from the amino acid sequence of ROS1 following the fusion junction with MYO5A, e.g., of the ROS1 sequence shown in SEQ ID NO: 12).

In one embodiment, the MYO5A-ROS1 fusion polypeptide includes a tyrosine kinase domain or a functional fragment thereof. In a related aspect, the invention features MYO5A-ROS1 fusion polypeptide or fragments operatively linked to heterologous polypeptides to form fusion proteins.

In another embodiment, the MYO5A-ROS1 fusion polypeptide or fragment is a peptide, e.g., an immunogenic peptide or protein that contains a fusion junction described herein. Such immunogenic peptides or proteins can be used to raise antibodies specific to the fusion protein. In other embodiments, such immunogenic peptides or proteins can be used for vaccine preparation. The vaccine preparation can include other components, e.g., an adjuvant.

In another aspect, the invention features antibody molecules that bind to a MYO5A-ROS1 fusion polypeptide or fragment described herein. In embodiments, the antibody can distinguish wild type ROS1 (or MYO5A) from MYO5A-ROS1.

### Detection Reagents and Detection of Mutations

In another aspect, the invention features a detection reagent, e.g., a purified or an isolated preparation thereof. Detection reagents can distinguish a nucleic acid, or protein sequence, having a breakpoint, e.g., a MYO5A-ROS1 breakpoint; from a reference sequence. In one embodiment, the detection reagent detects (e.g., specifically detects) a MYO5A-ROS1 fusion nucleic acid or a polypeptide, e.g., distinguishes a wild type ROS1 or another ROS1 fusion (or MYO5A) from a MYO5A-ROS1 nucleic acid (e.g., as described herein in SEQ ID NO:29 and SEQ ID NO: 11); or a MYO5A-ROS1 polypeptide (e.g., as described herein in SEQ ID NO:30 and SEQ ID NO: 12).

Detection reagents, e.g., nucleic acid-based detection reagents, can be used to identify mutations in a target nucleic acid. e.g., DNA, e.g., genomic DNA or cDNA, or RNA, e.g., in a sample, e.g., a sample of nucleic acid derived from a neoplastic or tumor cell, e.g., a melanocytic neoplasm, melanoma or metastatic cell. Detection reagents, e.g., antibody-based detection reagents, can be used to identify mutations in a target protein, e.g., in a sample, e.g., a sample of protein derived from, or produced by, a neoplastic or tumor cell, e.g., a melanocytic neoplasm, melanoma or metastatic cell.

### Method of Treatment

In a related aspect, a method of inhibiting, reducing, or treating a hyperproliferative disorder, e.g., a neoplasm (including benign, pre-malignant or malignant (e.g., a cancer), in a subject is provided. The method includes administering to the subject a preselected therapeutic agent, e.g., an anti-cancer agent (e.g., a kinase inhibitor), as a single agent, or in combination, in an amount sufficient to reduce, inhibit or treat the activity or expression of MYO5A-ROS1 (e.g., a MYO5A-ROS1 fusion described herein), thereby inhibiting, reducing, or treating the hyperproliferative disorder in the subject.

In one embodiment, the subject treated has a MYO5A-ROS1 fusion; e.g., the subject has a tumor or cancer harboring a MYO5A-ROS1 fusion. In other embodiments, the subject has been previously identified as having a MYO5A-ROS1 fusion. In yet other embodiments, the subject has been previously identified as being likely or unlikely to respond to treatment with a protein kinase inhibitor, e.g., a subject that has previously participated in a clinical trial. In other embodiments, the subject has been previously identified as being likely or unlikely to respond to treatment with a protein kinase inhibitor, based on the presence of the MYO5A-ROS1 fusion. In one embodiment, the subject is a mammal, e.g., a human. In one embodiment, the subject has, or at risk of having a cancer at any stage of disease. In other embodiments, the subject is a patient, e.g., a cancer patient.

In certain embodiments, the neoplasm or neoplastic cell is a benign, pre-malignant, malignant (cancer) or metastasis. In certain embodiments, the cancer is a solid tumor, a soft tissue tumor, or a metastatic lesion.

In one embodiment, the neoplasm or neoplastic cell is a benign, pre-malignant, malignant (cancer) or metastasis. In certain embodiments, the cancer is a solid tumor, a soft tissue tumor, or a metastatic lesion. In one embodiment, the cancer is a carcinoma. In one embodiment, the cancer is an adenocarcinoma. In an embodiment, the cancer is a lung adenocarcinoma. In one embodiment, the cancer is chosen from a lung cancer, a pancreatic cancer, melanoma, a colorectal cancer, an esophageal-gastric cancer, a thyroid cancer, or an adenocarcinoma. In one embodiment, the cancer is a lung adenocarcinoma. In other embodiment, the lung cancer is chosen from one or more of the following: non-small cell lung cancer (NSCLC), small cell lung cancer (SCLC), squamous cell carcinoma (SCC), adenocarcinoma of the lung, bronchogenic carcinoma, or a combination thereof. In one embodiment, the lung cancer is NSCLC or SCC.

In one embodiment, the anti-cancer agent is a ROS1 inhibitor. In one embodiment, the anti-cancer agent is a MYO5A inhibitor. In one embodiment, the anti-cancer agent is a kinase inhibitor. In one embodiment, the anti-cancer agent is a ROS1 inhibitor, e.g., Ganetespib; Crizotinib; TAE684; a dual ALK and ROS1 inhibitor.

### PWWP2A-ROS1 Fusions

In one embodiment, a fusion includes an in-frame fusion of an exon of PWWP domain containing 2A protein (PWWP2A), e.g., one more exons of PWWP2A (e.g., one or more of exon 1 of PWWP2A) or a fragment thereof, and an exon of C-Ros oncogene 1 (ROS1), e.g., one or more exons of a ROS1 (e.g., one or more of exons 36-43 of ROS1) or a fragment thereof. For example, the PWWP2A-ROS1 fusion can include an in-frame fusion within an intron of PWWP2A (e.g., intron 1) or a fragment thereof, with an intron of ROS1 (e.g., intron 35) or a fragment thereof. In one embodiment, the fusion of the PWWP2A-ROS1 fusion comprises the nucleotide sequence of: chromosome 5 at one or more of a nucleotide (plus or minus 10, 20, 30, 50, 60, 70, 80, 100 or more nucleotides) and chromosome 6 at one or more of a nucleotide (plus or minus 10, 20, 30, 50, 60, 70, 80, 100 or more nucleotides). In one embodiment, the PWWP2A-ROS1 fusion is a translocation, e.g., a translocation of a portion of chromosome 5 and a portion of chromosome 6.

In certain embodiments, the PWWP2A-ROS1 fusion is in a 5′-PWWP2A to 3′-ROS1 configuration (also referred to herein as “5′-PWWP2A-ROS1-3′).” The term “fusion” or “fusion molecule” can refer to a polypeptide or a nucleic acid fusion, depending on the context. It may include a full-length sequence of a fusion or a fragment thereof, e.g., a fusion junction (e.g., a fragment including a portion of PWWP2A and a portion of ROS1, e.g., a portion of the PWWP2A-ROS1 fusion described herein). In one embodiment, the PWWP2A-ROS1 fusion polypeptide includes a fragment of the amino acid sequence shown in SEQ ID NO:34 and a fragment of the amino acid sequence shown in SEQ ID NO: 12, or an amino acid sequence substantially identical thereto. In another embodiment, the PWWP2A-ROS1 fusion nucleic acid includes a fragment of the nucleotide sequence shown in SEQ ID NO:33 and a fragment of the nucleotide sequence shown in SEQ ID NO: 11, or a nucleotide sequence substantially identical thereto. In one embodiment, the PWWP2A-ROS1 fusion polypeptide comprises sufficient PWWP2A and sufficient ROS1 sequence such that the 5′ PWWP2A-3′ ROS1 fusion has kinase activity, e.g., has elevated activity, e.g., tyrosine kinase activity. In any event, the fusion causes activation in the cells harboring it of oncogenic signaling pathways.

In certain embodiments, the PWWP2A-ROS1 fusion comprises one or more (or all of) exon 1 from PWWP2A and one or more (or all of) exons 36-43 of ROS1 (e.g., one or more of the exons shown in SEQ ID NO:33 and 112 SEQ ID NO: 11. In another embodiment, the PWWP2A-ROS1 fusion comprises one or more (or all of) exon 1 of PWWP2A and one or more (or all of) exons 36-43 of ROS1. In certain embodiments, the PWWP2A-ROS1 fusion comprises at least 1 or more exons (or encoded exons) from PWWP2A and at least 1, 2, 3, 4, 5, 6, 7, 8 or more exons (or encoded exons) from ROS1 (e.g., from the PWWP2A and ROS1 sequences shown in SEQ ID NO:33 and SEQ ID NO:34 and SEQ ID NO:11 and SEQ ID NO:12.

In certain embodiments, the PWWP2A-ROS1 fusion comprises exon 1 or a fragment thereof from PWWP2A, and exons 36-43 or a fragment thereof from ROS1 (e.g., as shown in SEQ ID NO:33 and SEQ ID NO:11). In one embodiment, the PWWP2A-ROS1 fusion comprises at least 5, 10, 15, 20, 30, 40, 50 or more amino acids from exon 1 of PWWP2A (e.g., from the amino acid sequence of PWWP2A as shown in SEQ ID NO:34 (e.g., from the amino acid sequence of PWWP2A preceding the fusion junction with ROS1, and at least 5, 10, 15, 20, 30, 40, 50 or more amino acids from exons 36-43 of ROS1 (e.g., from the amino acid sequence of ROS1 as shown in SEQ ID NO:12). In another embodiment, the PWWP2A-ROS1 fusion comprises at least 6, 12, 15, 20, 25, 50, 75, 100 or more nucleotides from exon 1 of PWWP2A (e.g., from the nucleotide sequence of PWWP2A as shown in SEQ ID NO:33 (e.g., from the nucleotide sequence of PWWP2A preceding the fusion junction with ROS1; and at least 6, 12, 15, 20, 25, 50, 75, 100 or more nucleotides from exons 36-43 of ROS1 (e.g., from the nucleotide sequence of ROS1 as shown in SEQ ID NO: 11).

### PWWP2A-ROS1 Nucleic Acid Molecules

In one aspect, the invention features a nucleic acid molecule (e.g., an isolated or purified) nucleic acid molecule that includes a fragment of a PWWP2A gene and a fragment of a ROS1 gene. In one embodiment, the nucleotide sequence encodes a PWWP2A-ROS1 fusion polypeptide that includes a tyrosine kinase domain or a functional fragment thereof. In another embodiment, the nucleotide sequence encodes a fragment of the ROS1 polypeptide including the amino acid sequence of SEQ ID NO: 12 or a fragment thereof, or a sequence substantially identical thereto. In other embodiments, the nucleic acid molecule includes a fragment of the PWWP2A gene encoding the amino acid sequence of SEQ ID NO:34 or a fragment thereof, or a sequence substantially identical thereto. In yet other embodiments, the nucleic acid molecule includes a nucleotide sequence encoding the amino acid sequence shown in SEQ ID NO:34, or a fragment thereof, and the amino acid sequence shown in SEQ ID NO: 12 or a fragment thereof, or a sequence substantially identical thereto.

In one embodiment, the nucleic acid molecule includes a fusion, e.g., an in-frame fusion, between an intron of PWWP2A (e.g., intron 1, or a fragment thereof), and an intron of ROS1 (e.g., intron 35, or a fragment thereof). The PWWP2A-ROS1 fusion can comprise a fusion of the nucleotide sequence of: chromosome 5 at one or more of a nucleotide (plus or minus 10, 20, 30, 50, 60, 70, 80, 100 nucleotides) and chromosome 6 at one or more of a nucleotide (plus or minus 10, 20, 30, 50, 60, 70, 80, 100 nucleotides), or a fragment thereof. In one embodiment, the PWWP2A-ROS1 fusion comprises a fusion of the nucleotide sequence of: chromosome 5 at one or more of a nucleotide (plus or minus 10, 20, 30, 50, 60, 70, 80, 100 nucleotides) and chromosome 6 at one or more of a nucleotide (plus or minus 10, 20, 30, 50, 60, 70, 80, 100 nucleotides), or a fragment thereof.

In another embodiment, the PWWP2A-ROS1 fusion comprises a nucleotide sequence (e.g., a fragment of a nucleotide sequence) shown in SEQ ID NO:33 and a nucleotide sequence (e.g., a fragment of a nucleotide sequence) shown in SEQ ID NO: 11, or a fragment of the fusion. In one embodiment, the PWWP2A-ROS1 fusion comprises a nucleotide sequence substantially identical to the nucleotide sequence (e.g., a fragment of a nucleotide sequence) shown in SEQ ID NO:33 and the nucleotide sequence (e.g., a fragment of a nucleotide sequence) shown SEQ ID NO: 11, or a fragment of the fusion. In one embodiment, the PWWP2A-ROS1 fusion comprises a nucleotide sequence at least 70%, at least 75%, at least 80%, at least 85%, at least 90%, at least 95%, at least 99%, at least 99.5 or greater, identical to the nucleotide sequence (e.g., a fragment of a nucleotide sequence) shown in SEQ ID NO:33 and to the nucleotide sequence (e.g., a fragment of a nucleotide sequence) shown in SEQ ID NO: 11. In one embodiment, the PWWP2A-ROS1 fusion comprises a nucleotide sequence containing at least 25, 50, 100, 150, 200, 500, 1000, 1500, 2000, 2500, 3000, or more nucleotides of the nucleotide sequence shown in SEQ ID NO:33 and a nucleotide sequence containing at least 25, 50, 100, 150, 200, 500, 1000, 1500, 2000, 2500, 3000, or more nucleotides of the nucleotide sequence shown in SEQ ID NO: 11. In one embodiment, the PWWP2A-ROS1 fusion comprises a nucleotide sequence containing at least 25, 50, 100, 150, 200, 500, 1000, 1500, 2000, 2500, 3000, or more contiguous nucleotides of the nucleotide sequence shown in SEQ ID NO:33 and a nucleotide sequence containing at least 25, 50, 100, 150, 200, 500, 1000, 1500, 2000, 2500, 3000, or more nucleotides of the nucleotide sequence shown in SEQ ID NO: 11.

In another embodiment, the nucleic acid molecule includes a fusion, e.g., an in-frame fusion, of at least 1 or more exons of PWWP2A or a fragment thereof (e.g., one or more of exon 1 of PWWP2A or a fragment thereof), and at least 1, 2, 3, 4, 5, 6, 7, 8 or more exons of ROS1 or a fragment thereof (e.g., one or more of exons 36-43 of ROS1 or a fragment thereof). In yet other embodiments, the nucleic acid molecule includes a fragment the nucleotide sequence shown in SEQ ID NO:33 and a fragment of the nucleotide sequence shown in SEQ ID NO:11 or a fragment of the fusion, or a sequence substantially identical thereto.

In one embodiment, the nucleic acid molecule is complementary to at least a portion of a nucleotide sequence disclosed herein, e.g., is capable of hybridizing under a stringency condition described herein to SEQ ID NO:33 and/or SEQ ID NO: 11, or a fragment thereof. In yet another embodiment, the nucleic acid molecule hybridizes to a nucleotide sequence that is complementary to at least a portion of a nucleotide sequence disclosed herein, e.g., is capable of hybridizing under a stringency condition to a nucleotide sequence complementary to SEQ ID NO:33 and/or SEQ ID NO: 11, or a fragment thereof. The nucleotide sequence of a cDNA encoding an exemplary 5′ PWWP2A-3′ ROS1 fusion is shown in at least exon 1 (e.g., exon 1) of SEQ ID NO:33 and at least exon 36 (e.g., exons 36-43) of SEQ ID NO: 11, and the predicted amino acid sequence is shown in the corresponding encoded exons of SEQ ID NO: 34 and the corresponding encoded exons of SEQ ID NO: 12, respectively.

In an embodiment the PWWP2A-ROS1 nucleic acid molecule comprises sufficient PWWP2A and sufficient ROS1 sequence such that the encoded 5′ PWWP2A-3′ ROS1 fusion has kinase activity, e.g., has elevated activity. In certain embodiments, the 5′ PWWP2A-3′ ROS1 fusion comprises exon 1 from PWWP2A and exons 36-43 from ROS1. In certain embodiments, the PWWP2A-ROS1 fusion comprises at least 1 or more exons from PWWP2A and at least at least 1, 2, 3, 4, 5, 6, 7, 8 or more exons of ROS1. In certain embodiments, the PWWP2A-ROS1 fusion comprises a fusion of exon 1 from PWWP2A and exon 36 from ROS1. In another embodiment, the PWWP2A-ROS1 fusion comprises 1 or more exons of PWWP2A; and at least 1, 2, 3, 4, 5, 6, 7, 8 or more exons of ROS1.

In one embodiment, the nucleic acid molecule includes a nucleotide sequence that has an in-frame fusion of intron 1 of PWWP2A (e.g., NM_{—}052927) with intron 35 of ROS1 (e.g., NM_{—}002944). In another embodiment, the nucleic acid molecule includes a nucleotide sequence that includes a breakpoint. For example, the nucleic acid molecule includes a nucleotide sequence that includes the fusion junction between the PWWP2A gene and the ROS1 gene. e.g., the breakpoint between intron 1 of PWWP2A and intron 35 of ROS1. In other embodiments, the nucleic acid molecules includes a nucleotide sequence of one or more of nucleotide of chromosome 5 coupled to (e.g., directly or indirectly juxtaposed to) one or more of nucleotide of chromosome 6. In one embodiment, the nucleic acid molecule includes the nucleotide sequence of: chromosome 5 at one or more of a nucleotide plus or minus 10, 20, 30, 40 50, 60, 80, 100, 150 nucleotides and chromosome 6 at one or more of a nucleotide plus or minus 10, 20, 30, 40 50, 60, 80, 100, 150 nucleotides (corresponding to the breakpoint of a PWWP2A-ROS1 fusion), or a fragment thereof, or a sequence substantially identical thereto. In one embodiment, the nucleic acid molecule is complementary to at least a portion of a nucleotide sequence disclosed herein, e.g., is capable of hybridizing under a stringency condition described herein to SEQ ID NO:33 and/or SEQ ID NO:11 or a fragment thereof. In yet other embodiment, the nucleic acid molecule hybridizes to a nucleotide sequence that is complementary to at least a portion of a nucleotide sequence disclosed herein, e.g., is capable of hybridizing under a stringency condition described herein to a nucleotide sequence complementary to SEQ ID NO:33 or SEQ ID NO: 11 or a fragment thereof.

In another embodiment, the PWWP2A-ROS1 fusion nucleic acid comprises at least 6, 12, 15, 20, 25, 50, 75, 100 or more nucleotides from exon 1 of PWWP2A (e.g., from the nucleotide sequence of PWWP2A preceding the fusion junction with ROS1, e.g., of the PWWP2A sequence shown in SEQ ID NO:33), and at least 6, 12, 15, 20, 25, 50, 75, 100 or more nucleotides from exon 36 of ROS1 (e.g., from the nucleotide sequence of ROS1 following the fusion junction with PWWP2A, e.g., of the ROS1 sequence shown in SEQ ID NO: 11).

In other embodiments, the nucleic acid molecule includes a nucleotide sequence encoding a PWWP2A-ROS1 fusion polypeptide that includes a fragment of a PWWP2A gene and a fragment of a ROS1 gene. In one embodiment, the nucleotide sequence encodes a PWWP2A-ROS1 fusion polypeptide that includes e.g., a tyrosine kinase domain or a functional fragment thereof. In yet other embodiments, the nucleic acid molecule includes a nucleotide sequence encoding the amino acid sequence (e.g., a fragment of the amino acid sequence) shown in SEQ ID NO:34 and a nucleotide sequence encoding the amino acid sequence (e.g., a fragment of the amino acid sequence) shown in SEQ ID NO:12, or a fragment of the fusion, or a sequence substantially identical thereto. In one embodiment, the encoded PWWP2A-ROS1 fusion polypeptide includes a tyrosine kinase domain or a functional fragment thereof.

In one embodiment, the PWWP2A-ROS1 fusion includes the full sequence or a fragment of the nucleotide sequence shown in SEQ ID NO:31 and SEQ ID NO:90, or a nucleotide sequence substantially identical thereto. In another embodiment, the PWWP2A-ROS1 fusion polypeptide includes the full sequence or a fragment of the amino acid sequence shown in SEQ ID NO:32 and SEQ ID NO:91, or an amino acid sequence substantially identical thereto, for example at least 70% or at least 80% or at least 90% identical or even more.

In a related aspect, the invention features nucleic acid constructs that include the PWWP2A-ROS1 nucleic acid molecules described herein. In certain embodiments, the nucleic acid molecules are operatively linked to a native or a heterologous regulatory sequence. Also included are vectors and host cells that include the PWWP2A-ROS1 nucleic acid molecules described herein, e.g., vectors and host cells suitable for producing the nucleic acid molecules and polypeptides described herein.

In a related aspect, methods of producing the nucleic acid molecules and polypeptides described herein are also described.

In another aspect, the invention features nucleic acid molecules that reduce or inhibit the expression of a nucleic acid molecule that encodes a PWWP2A-ROS1 fusion described herein. Examples of such nucleic acid molecules include, for example, antisense molecules, ribozymes, RNAi, triple helix molecules that hybridize to a nucleic acid encoding PWWP2A-ROS1, or a transcription regulatory region of PWWP2A-ROS1, and blocks or reduces mRNA expression of PWWP2A-ROS1.

### Nucleic Acid Detection and Capturing Reagents

The invention also features a nucleic acid molecule, e.g., nucleic acid fragment, suitable as probe, primer, bait or library member that includes, flanks, hybridizes to, which are useful for identifying, or are otherwise based on, the PWWP2A-ROS1 fusions described herein. In certain embodiments, the probe, primer or bait molecule is an oligonucleotide that allows capture, detection or isolation of a PWWP2A-ROS1 fusion nucleic acid molecule described herein. The oligonucleotide can comprise a nucleotide sequence substantially complementary to a fragment of the PWWP2A-ROS1 fusion nucleic acid molecules described herein. The sequence identity between the nucleic acid fragment, e.g., the oligonucleotide, and the target PWWP2A-ROS1 sequence need not be exact, so long as the sequences are sufficiently complementary to allow the capture, detection or isolation of the target sequence. In one embodiment, the nucleic acid fragment is a probe or primer that includes an oligonucleotide between about 5 and 25, e.g., between 10 and 20, or 10 and 15 nucleotides in length. In other embodiments, the nucleic acid fragment is a bait that includes an oligonucleotide between about 100 to 300 nucleotides, 130 and 230 nucleotides, or 150 and 200 nucleotides, in length.

In one embodiment, the nucleic acid fragment can be used to identify or capture, e.g., by hybridization, a PWWP2A-ROS1 fusion. For example, the nucleic acid fragment can be a probe, a primer, or a bait, for use in identifying or capturing, e.g., by hybridization, a PWWP2A-ROS1 fusion described herein. In one embodiment, the nucleic acid fragment can be useful for identifying or capturing a PWWP2A-ROS1 breakpoint, e.g., the nucleotide sequence of: chromosome 5 at nucleotide plus or minus 10, 20, 30, 40, 50, 60, 80, 100, 150 nucleotides and chromosome 6 at nucleotide plus or minus 10, 20, 30, 40, 50, 60, 80, 100, 150 nucleotides.

In one embodiment, the nucleic acid fragment hybridizes to a nucleotide sequence within a chromosomal rearrangement that creates an in-frame fusion of intron 1 of PWWP2A with intron 35 of ROS1. In one embodiment, the nucleic acid fragment hybridizes to a nucleotide sequence in the region In other embodiments, the nucleic acid molecules includes a nucleotide sequence in the region of a nucleotide sequence of chromosome 5 coupled to (e.g., juxtaposed to) nucleotides in the region of a nucleotide sequence of chromosome 6. In one embodiment, the nucleic acid fragment hybridizes to a nucleotide sequence that includes a breakpoint, e.g., the nucleotide sequence of: chromosome 5 at nucleotide plus or minus 10, 20, 30, 40 50, 60, 80, 100, 150 or more nucleotides and chromosome 6 at nucleotide plus or minus 10, 20, 30, 40 50, 60, 80, 100, 150 or more nucleotides. For example, the nucleic acid fragment can hybridize to a nucleotide sequence that includes the fusion junction between the PWWP2A gene and the ROS1 gene, e.g., a nucleotide sequence that includes a portion of a nucleotide sequence within intron 1 of a PWWP2A gene and intron 35 of a ROS1 gene.

In another embodiment, the nucleic acid fragment hybridizes to a nucleotide sequence that comprises at least 6, 12, 15, 20, 25, 50, 75, 100, 150 or more nucleotides from exon 1 of PWWP2A (e.g., from the nucleotide sequence of PWWP2A preceding the fusion junction with ROS1, e.g., of the PWWP2A sequence shown in SEQ ID NO:33), and at least 6, 12, 15, 20, 25, 50, 75, 100, 150 or more nucleotides from exon 36 of ROS1 (e.g., from the nucleotide sequence of ROS1 following the fusion junction with PWWP2A, e.g., of the ROS1 sequence shown in SEQ ID NO: 11).

The probes or primers described herein can be used, for example, for FISH detection or PCR amplification. In one exemplary embodiment where detection is based on PCR, amplification of the PWWP2A-ROS1 fusion junction fusion junction can be performed using a primer or a primer pair, e.g., for amplifying a sequence flanking the fusion junctions described herein, e.g., the mutations or the junction of a chromosomal rearrangement described herein, e.g., PWWP2A-ROS1.

In one embodiment, a pair of isolated oligonucleotide primers can amplify a region containing or adjacent to a position in the PWWP2A-ROS1 fusion. For example, forward primers can be designed to hybridize to a nucleotide sequence within PWWP2A genomic or mRNA sequence (e.g., a nucleotide sequence within exon 1 of PWWP2A of SEQ ID NO:33, and the reverse primers can be designed to hybridize to a nucleotide sequence of ROS1 (e.g., a nucleotide sequence within exon 36 of ROS1, of SEQ ID NO:11.

In another embodiment, the nucleic acid fragments can be used to identify, e.g., by hybridization, a PWWP2A-ROS1 fusion. In one embodiment, the nucleic acid fragment hybridizes to a nucleotide sequence that includes a fusion junction between the PWWP2A transcript and the ROS1 transcript.

In other embodiments, the nucleic acid fragment includes a bait that comprises a nucleotide sequence that hybridizes to a PWWP2A-ROS1 fusion nucleic acid molecule described herein, and thereby allows the capture or isolation said nucleic acid molecule. In one embodiment, a bait is suitable for solution phase hybridization. In other embodiments, a bait includes a binding entity, e.g., an affinity tag, that allows capture and separation, e.g., by binding to a binding entity, of a hybrid formed by a bait and a nucleic acid hybridized to the bait.

In other embodiments, the nucleic acid fragment includes a library member comprising a PWWP2A-ROS1 nucleic acid molecule described herein. In one embodiment, the library member includes a rearrangement that results in a PWWP2A-ROS1 fusion described herein.

The nucleic acid fragment can be detectably labeled with, e.g., a radiolabel, a fluorescent label, a bioluminescent label, a chemiluminescent label, an enzyme label, a binding pair label, or can include an affinity tag; a tag, or identifier (e.g., an adaptor, barcode or other sequence identifier).

### PWWP2A-ROS1 Fusion Polypeptides

In another embodiment, the PWWP2A-ROS1 fusion comprises an amino acid sequence (e.g., a fragment of the amino acid sequence) shown in SEQ ID NO:34 and an amino acid sequence (e.g., a fragment of the amino acid sequence) shown in SEQ ID NO:12, or a fragment of the fusion. In one embodiment, the PWWP2A-ROS1 fusion comprises an amino acid sequence substantially identical to the amino acid sequence (e.g., a fragment of the amino acid sequence) shown in SEQ ID NO:34 and the amino acid sequence (e.g., a fragment of the amino acid sequence) shown in SEQ ID NO: 12, or a fragment thereof. In one embodiment, the PWWP2A-ROS1 fusion comprises an amino acid sequence at least 70%, at least 75%, at least 80%, at least 85%, at least 90%, at least 95%, at least 99%, at least 99.5 or greater, identical to the amino acid sequence (e.g., a fragment of the amino acid sequence) shown in SEQ ID NO:34 and the amino acid sequence (e.g., a fragment of the amino acid sequence) shown in SEQ ID NO:12. In one embodiment, the PWWP2A-ROS1 fusion comprises a sequence containing at least 10, 20, 50, 100, 500, 600, 700, 800, 900, 1000, or more amino acids of the amino acid sequence shown in SEQ ID NO:34 and SEQ ID NO: 12. In one embodiment, the PWWP2A-ROS1 fusion comprises an amino acid sequence containing at least 5, 10, 20, 50, 100, 500, 600, 700, 800, 900, 1000, or more contiguous amino acids of the amino acid sequence shown in SEQ ID NO:34 and at least 5, 10, 20, 50, 100, 500, 600, 700, 800, 900, 1000, or more contiguous amino acids of the amino acid sequence shown in SEQ ID NO: 12. In one embodiment, the 5′ PWWP2A-3′ ROS1 fusion polypeptide includes a receptor tyrosine kinase domain or a functional fragment thereof. In an embodiment, the 5′PWWP2A-3′ROS1 fusion polypeptide comprises sufficient ROS1 and sufficient PWWP2A sequence such that it has kinase activity, e.g., has elevated activity as stated above.

In another aspect, the invention features a PWWP2A-ROS1 fusion polypeptide (e.g., a purified PWWP2A-ROS1 fusion polypeptide), a biologically active or antigenic fragment thereof, as well as reagents (e.g., antibody molecules that bind to a PWWP2A-ROS1 fusion polypeptide), methods for modulating a PWWP2A-ROS1 polypeptide activity and detection of a PWWP2A-ROS1 polypeptide.

In one embodiment, the PWWP2A-ROS1 fusion polypeptide has at least one biological activity. In one embodiment, at least one biological activity of the PWWP2A-ROS1 fusion polypeptide is reduced or inhibited by an anti-cancer drug, e.g., a PWWP2A inhibitor, a ROS1 inhibitor. In one embodiment, at least one biological activity of the PWWP2A-ROS1 fusion polypeptide is reduced or inhibited by a ROS1 inhibitor. In one embodiment, at least one biological activity of the PWWP2A-ROS1 fusion polypeptide is reduced or inhibited by a PWWP2A inhibitor. In one embodiment, at least one biological activity of the PWWP2A-ROS1 fusion polypeptide is reduced or inhibited by a ROS1 inhibitor, e.g., Ganetespib; Crizotinib; TAE684; a dual ALK and ROS1 inhibitor.

In yet other embodiments, the PWWP2A-ROS1 fusion polypeptide is encoded by a nucleic acid molecule described herein. In one embodiment, the PWWP2A-ROS1 fusion polypeptide is encoded by an in-frame fusion of intron 1 of PWWP2A with intron 35 of ROS1 (e.g., a sequence on chromosome 5 and a sequence on chromosome 6). In another embodiment, the PWWP2A-ROS1 fusion polypeptide includes an amino acid sequence encoded by a nucleotide sequence comprising a fusion junction between the PWWP2A transcript and the ROS1 transcript.

In certain embodiments, the PWWP2A-ROS1 fusion polypeptide comprises one or more of encoded exon 1 from PWWP2A and one or more of encoded exons 36-43 of ROS1. In certain embodiments, the PWWP2A-ROS1 fusion polypeptide comprises at least 1 or more encoded exons of PWWP2A and at least at least 1, 2, 3, 4, 5, 6, 7, 8 or more encoded exons of ROS1. In certain embodiments, the PWWP2A-ROS1 fusion polypeptide comprises a fusion of encoded exon 1 from PWWP2A and encoded exon 36 from ROS1 (or a fragment thereof). In other embodiments, the fusion comprises at least 1 or more encoded exons of PWWP2A; and at least 1, 2, 3, 4, 5, 6, 7, 8 or more encoded exons of ROS1. In certain embodiments, the PWWP2A-ROS1 fusion polypeptide comprises encoded exon 1 from PWWP2A and exons 36-43 of ROS1. In certain embodiments, the 5′ PWWP2A-3′ ROS1 fusion polypeptide comprises a fusion junction of the sequence of exon 1 from PWWP2A and the sequence of exon 36 from ROS1.

In certain embodiments, the PWWP2A-ROS1 fusion comprises the amino acid sequence corresponding to exon 1 or a fragment thereof from PWWP2A, and the amino acid sequence corresponding to exon 36 or a fragment thereof from ROS1 (e.g., as shown in SEQ ID NO:34 and SEQ ID NO:12). In one embodiment, the PWWP2A-ROS1 fusion comprises at least 5, 10, 15, 20 or more amino acids from exon 1 of PWWP2A (e.g., from the amino acid sequence of PWWP2A preceding the fusion junction with ROS1, e.g., of the PWWP2A sequence shown in SEQ ID NO:34), and at least 5, 10, 15, 20 or more amino acids from exon 36 of ROS1 (e.g., from the amino acid sequence of ROS1 following the fusion junction with PWWP2A, e.g., of the ROS1 sequence shown in SEQ ID NO: 12).

In one embodiment, the PWWP2A-ROS1 fusion polypeptide includes a tyrosine kinase domain or a functional fragment thereof. In a related aspect, the invention features PWWP2A-ROS1 fusion polypeptide or fragments operatively linked to heterologous polypeptides to form fusion proteins.

In another embodiment, the PWWP2A-ROS1 fusion polypeptide or fragment is a peptide, e.g., an immunogenic peptide or protein. Such a peptide or protein contains a fusion junction described herein. Such immunogenic peptides or proteins can be used to raise antibodies specific to the fusion protein. In other embodiments, such immunogenic peptides or proteins can be used for vaccine preparation. The vaccine preparation can include other components, e.g., an adjuvant.

In another aspect, the invention features antibody molecules that bind to a PWWP2A-ROS1 fusion polypeptide or fragment described herein. In embodiments, the antibody can distinguish wild type ROS1 (or PWWP2A) from PWWP2A-ROS1.

### Detection Reagents and Detection of Mutations

In another aspect, the invention features a detection reagent, e.g., a purified or an isolated preparation thereof. Detection reagents can distinguish a nucleic acid, or protein sequence, having a breakpoint, e.g., a PWWP2A-ROS1 breakpoint; from a reference sequence. In one embodiment, the detection reagent detects (e.g., specifically detects) a PWWP2A-ROS1 fusion nucleic acid or a polypeptide, e.g., distinguishes a wild type ROS1 or another ROS1 fusion (or PWWP2A) from a PWWP2A-ROS1 nucleic acid (e.g., as described herein in SEQ ID NO:33 and SEQ ID NO:11); or a PWWP2A-ROS1 polypeptide (e.g., as described herein in SEQ ID NO:34 and SEQ ID NO:12).

Detection reagents, e.g., nucleic acid-based detection reagents, can be used to identify mutations in a target nucleic acid. e.g., DNA, e.g., genomic DNA or cDNA, or RNA, e.g., in a sample, e.g., a sample of nucleic acid derived from a neoplastic or tumor cell, e.g., a melanocytic neoplasm, melanoma or metastatic cell. Detection reagents, e.g., antibody-based detection reagents, can be used to identify mutations in a target protein, e.g., in a sample, e.g., a sample of protein derived from, or produced by, a neoplastic or tumor cell, e.g., a melanocytic neoplasm, melanoma or metastatic cell.

### Method of Treatment

In a related aspect, a method of inhibiting, reducing, or treating a hyperproliferative disorder, e.g., a neoplasm (including benign, pre-malignant or malignant (e.g., a cancer), in a subject is provided. The method includes administering to the subject a preselected therapeutic agent, e.g., an anti-cancer agent (e.g., a kinase inhibitor), as a single agent, or in combination, in an amount sufficient to reduce, inhibit or treat the activity or expression of PWWP2A-ROS1 (e.g., a PWWP2A-ROS1 fusion described herein), thereby inhibiting, reducing, or treating the hyperproliferative disorder in the subject.

In one embodiment, the subject treated has a PWWP2A-ROS1 fusion; e.g., the subject has a tumor or cancer harboring a PWWP2A-ROS1 fusion. In other embodiments, the subject has been previously identified as having a PWWP2A-ROS1 fusion. In yet other embodiments, the subject has been previously identified as being likely or unlikely to respond to treatment with a protein kinase inhibitor, e.g., a subject that has previously participated in a clinical trial. In other embodiments, the subject has been previously identified as being likely or unlikely to respond to treatment with a protein kinase inhibitor, based on the presence of the PWWP2A-ROS1 fusion. In one embodiment, the subject is a mammal, e.g., a human. In one embodiment, the subject has, or at risk of having a cancer at any stage of disease. In other embodiments, the subject is a patient, e.g., a cancer patient.

In certain embodiments, the neoplasm or neoplastic cell is a benign, pre-malignant, malignant (cancer) or metastasis. In certain embodiments, the cancer is a solid tumor, a soft tissue tumor, or a metastatic lesion.

In one embodiment, the neoplasm or neoplastic cell is a benign, pre-malignant, malignant (cancer) or metastasis. In certain embodiments, the cancer is a solid tumor, a soft tissue tumor, or a metastatic lesion. In one embodiment, the cancer is a carcinoma. In one embodiment, the cancer is an adenocarcinoma. In an embodiment, the cancer is a lung adenocarcinoma. In one embodiment, the cancer is chosen from a lung cancer, a pancreatic cancer, melanoma, a colorectal cancer, an esophageal-gastric cancer, a thyroid cancer, or an adenocarcinoma. In one embodiment, the cancer is a lung adenocarcinoma. In other embodiment, the lung cancer is chosen from one or more of the following: non-small cell lung cancer (NSCLC), small cell lung cancer (SCLC), squamous cell carcinoma (SCC), adenocarcinoma of the lung, bronchogenic carcinoma, or a combination thereof. In one embodiment, the lung cancer is NSCLC or SCC.

In one embodiment, the anti-cancer agent is a ROS1 inhibitor. In one embodiment, the anti-cancer agent is a PWWP2A inhibitor. In one embodiment, the anti-cancer agent is a kinase inhibitor. In one embodiment, the anti-cancer agent is a ROS1 inhibitor, e.g., Ganetespib; Crizotinib; TAE684; a dual ALK and ROS1 inhibitor.

### TPM3-ALK Fusions

In one embodiment, a fusion includes an in-frame fusion of an exon of tropomyosin 3 (TPM3), e.g., one more exons of TPM3 (e.g., one or more of exons 1-8 of TPM3) or a fragment thereof, and an exon of anaplastic lymphoma receptor tyrosine kinase (ALK), e.g., one or more exons of an ALK (e.g., one or more of exons 20-29 of ALK) or a fragment thereof. For example, the TPM3-ALK fusion can include an in-frame fusion within an intron of TPM3 (e.g., intron 8) or a fragment thereof, with an intron of ALK (e.g., intron 19) or a fragment thereof. In one embodiment, the fusion of the TPM3-ALK fusion comprises the nucleotide sequence of: chromosome 1 at one or more of a nucleotide (plus or minus 10, 20, 30, 50, 60, 70, 80, 100 or more nucleotides) and chromosome 2 at one or more of a nucleotide (plus or minus 10, 20, 30, 50, 60, 70, 80, 100 or more nucleotides). In one embodiment, the TPM3-ALK fusion is a translocation, e.g., a translocation of a portion of chromosome 1 and a portion of chromosome 2.

In certain embodiments, the TPM3-ALK fusion is in a 5′-TPM3 to 3′-ALK configuration (also referred to herein as “5′-TPM3-ALK-3′).” The term “fusion” or “fusion molecule” can refer to a polypeptide or a nucleic acid fusion, depending on the context. It may include a full-length sequence of a fusion or a fragment thereof, e.g., a fusion junction (e.g., a fragment including a portion of TPM3 and a portion of ALK, e.g., a portion of the TPM3-ALK fusion described herein). In one embodiment, the TPM3-ALK fusion polypeptide includes a fragment of the amino acid sequence shown in SEQ ID NO:10 and a fragment of the amino acid sequence shown in SEQ ID NO:7, or an amino acid sequence substantially identical thereto. In another embodiment, the TPM3-ALK fusion nucleic acid includes a fragment of the nucleotide sequence shown in SEQ ID NO:9 and a fragment of the nucleotide sequence shown in SEQ ID NO:7, or a nucleotide sequence substantially identical thereto. In one embodiment, the TPM3-ALK fusion polypeptide comprises sufficient TPM3 and sufficient ALK sequence such that the 5′ TPM3-3′ ALK fusion has kinase activity, e.g., has elevated activity, e.g., tyrosine kinase activity. In any event, the fusion causes activation in the cells harboring it of oncogenic signaling pathways.

In certain embodiments, the TPM3-ALK fusion comprises one or more (or all of) exons 1-8 from TPM3 and one or more (or all of) exons 20-29 of ALK (e.g., one or more of the exons shown in SEQ ID NO:9 and SEQ ID NO:7. In another embodiment, the TPM3-ALK fusion comprises one or more (or all of) exons 1-8 of TPM3 and one or more (or all of) exons 20-29 of ALK. In certain embodiments, the TPM3-ALK fusion comprises at least 1, 2, 3, 4, 5, 6, 7, 8 or more exons (or encoded exons) from TPM3 and at least 1, 2, 3, 4, 5, 6, 7, 8, 4, 5, 6, 7, 8, 9, 10 or more exons (or encoded exons) from ALK (e.g., from the TPM3 and ALK sequences shown in SEQ ID NO:9 and SEQ ID NO:10 and SEQ ID NO:7 and SEQ ID NO:8.

In certain embodiments, the TPM3-ALK fusion comprises exons 1-8 or a fragment thereof from TPM3, and exons 20-29 or a fragment thereof from ALK (e.g., as shown in SEQ ID NO:9 and SEQ ID NO:7). In one embodiment, the TPM3-ALK fusion comprises at least 5, 10, 15, 20, 30, 40, 50 or more amino acids from exons 1-8 of TPM3 (e.g., from the amino acid sequence of TPM3 as shown in SEQ ID NO:10 (e.g., from the amino acid sequence of TPM3 preceding the fusion junction with ALK, and at least 5, 10, 15, 20, 30, 40, 50 or more amino acids from exons 20-29 of ALK (e.g., from the amino acid sequence of ALK as shown in SEQ ID NO:7). In another embodiment, the TPM3-ALK fusion comprises at least 6, 12, 15, 20, 25, 50, 75, 100 or more nucleotides from exons 1-8 of TPM3 (e.g., from the nucleotide sequence of TPM3 as shown in SEQ ID NO:9 (e.g., from the nucleotide sequence of TPM3 preceding the fusion junction with ALK); and at least 6, 12, 15, 20, 25, 50, 75, 100 or more nucleotides from exons 20-29 of ALK (e.g., from the nucleotide sequence of ALK as shown in SEQ ID NO:7).

In one embodiment, the TPM3-ALK fusion includes the full sequence or a fragment of the nucleotide sequence shown in SEQ ID NO:43 and SEQ ID NO:98, or a nucleotide sequence substantially identical thereto. In another embodiment, the TPM3-ALK fusion polypeptide includes the full sequence or a fragment of the amino acid sequence shown in SEQ ID NO:44 and SEQ ID NO:99, or an amino acid sequence substantially identical thereto, for example at least 70%, or at least 805 or at least 90% identical or even more.

### TPM3-ALK Nucleic Acid Molecules

In one aspect, the invention features a nucleic acid molecule (e.g., an isolated or purified) nucleic acid molecule that includes a fragment of a TPM3 gene and a fragment of an ALK gene. In one embodiment, the nucleotide sequence encodes a TPM3-ALK fusion polypeptide that includes a tyrosine kinase domain or a functional fragment thereof. In another embodiment, the nucleotide sequence encodes a fragment of the ALK polypeptide including the amino acid sequence of SEQ ID NO:7 or a fragment thereof, or a sequence substantially identical thereto. In other embodiments, the nucleic acid molecule includes a fragment of the TPM3 gene encoding the amino acid sequence of SEQ ID NO:10 or a fragment thereof, or a sequence substantially identical thereto. In yet other embodiments, the nucleic acid molecule includes a nucleotide sequence encoding the amino acid sequence shown in SEQ ID NO:10, or a fragment thereof, and the amino acid sequence shown in SEQ ID NO:7 or a fragment thereof, or a sequence substantially identical thereto.

In one embodiment, the nucleic acid molecule includes a fusion, e.g., an in-frame fusion, between an intron of TPM3 (e.g., intron 8, or a fragment thereof), and an intron of ALK (e.g., intron 19, or a fragment thereof). The TPM3-ALK fusion can comprise a fusion of the nucleotide sequence of: chromosome 1 at one or more of a nucleotide (plus or minus 10, 20, 30, 50, 60, 70, 80, 100 nucleotides) and chromosome 2 at one or more of a nucleotide (plus or minus 10, 20, 30, 50, 60, 70, 80, 100 nucleotides), or a fragment thereof. In one embodiment, the TPM3-ALK fusion comprises a fusion of the nucleotide sequence of: chromosome 1 at one or more of a nucleotide (plus or minus 10, 20, 30, 50, 60, 70, 80, 100 nucleotides) and chromosome 2 at one or more of a nucleotide (plus or minus 10, 20, 30, 50, 60, 70, 80, 100 nucleotides), or a fragment thereof.

In another embodiment, the TPM3-ALK fusion comprises a nucleotide sequence (e.g., a fragment of a nucleotide sequence) shown in SEQ ID NO:9 and a nucleotide sequence (e.g., a fragment of a nucleotide sequence) shown in SEQ ID NO:7, or a fragment of the fusion. In one embodiment, the TPM3-ALK fusion comprises a nucleotide sequence substantially identical to the nucleotide sequence (e.g., a fragment of a nucleotide sequence) shown in SEQ ID NO:9 and the nucleotide sequence (e.g., a fragment of a nucleotide sequence) shown SEQ ID NO:7, or a fragment of the fusion. In one embodiment, the TPM3-ALK fusion comprises a nucleotide sequence at least 70%, at least 75%, at least 80%, at least 85%, at least 90%, at least 95%, at least 99%, at least 99.5 or greater, identical to the nucleotide sequence (e.g., a fragment of a nucleotide sequence) shown in SEQ ID NO:9 and to the nucleotide sequence (e.g., a fragment of a nucleotide sequence) shown in SEQ ID NO:7. In one embodiment, the TPM3-ALK fusion comprises a nucleotide sequence containing at least 25, 50, 100, 150, 200, 500, 1000, 1500, 2000, 2500, 3000, or more nucleotides of the nucleotide sequence shown in SEQ ID NO:9 and a nucleotide sequence containing at least 25, 50, 100, 150, 200, 500, 1000, 1500, 2000, 2500, 3000, or more nucleotides of the nucleotide sequence shown in SEQ ID NO:7. In one embodiment, the TPM3-ALK fusion comprises a nucleotide sequence containing at least 25, 50, 100, 150, 200, 500, 1000, 1500, 2000, 2500, 3000, or more contiguous nucleotides of the nucleotide sequence shown in SEQ ID NO:9 and a nucleotide sequence containing at least 25, 50, 100, 150, 200, 500, 1000, 1500, 2000, 2500, 3000, or more nucleotides of the nucleotide sequence shown in SEQ ID NO:7.

In another embodiment, the nucleic acid molecule includes a fusion, e.g., an in-frame fusion, of at least 1, 2, 3, 4, 5, 6, 7, 8 or more exons of TPM3 or a fragment thereof (e.g., one or more of exons 1-8 of TPM3 or a fragment thereof), and at least 1, 2, 3, 4, 5, 6, 7, 8, 4, 5, 6, 7, 8, 9, 10 or more exons of ALK or a fragment thereof (e.g., one or more of exons 20-29 of ALK or a fragment thereof). In yet other embodiments, the nucleic acid molecule includes a fragment the nucleotide sequence shown in SEQ ID NO:9 and a fragment of the nucleotide sequence shown in SEQ ID NO:7 or a fragment of the fusion, or a sequence substantially identical thereto.

In one embodiment, the nucleic acid molecule is complementary to at least a portion of a nucleotide sequence disclosed herein, e.g., is capable of hybridizing under a stringency condition described herein to SEQ ID NO:9 and/or SEQ ID NO:7, or a fragment thereof. In yet another embodiment, the nucleic acid molecule hybridizes to a nucleotide sequence that is complementary to at least a portion of a nucleotide sequence disclosed herein, e.g., is capable of hybridizing under a stringency condition to a nucleotide sequence complementary to SEQ ID NO:9 and/or SEQ ID NO:7, or a fragment thereof. The nucleotide sequence of a cDNA encoding an exemplary 5′ TPM3-3′ ALK fusion is shown in at least exon 8 (e.g., exons 1-8) of SEQ ID NO:9 and at least exon 20 (e.g., exons 20-29) of SEQ ID NO:7, and the predicted amino acid sequence is shown in the corresponding encoded exons of SEQ ID NO: 10 and the corresponding encoded exons of SEQ ID NO:7, respectively.

In an embodiment the TPM3-ALK nucleic acid molecule comprises sufficient TPM3 and sufficient ALK sequence such that the encoded 5′ TPM3-3′ ALK fusion has kinase activity, e.g., has elevated activity. In any event, the fusion causes activation in the cells harboring it of oncogenic signaling pathways. In certain embodiments, the 5′ TPM3-3′ ALK fusion comprises exons 1-8 from TPM3 and exons 20-29 from ALK. In certain embodiments, the TPM3-ALK fusion comprises at least 1, 2, 3, 4, 5, 6, 7, 8 or more exons from TPM3 and at least at least 1, 2, 3, 4, 5, 6, 7, 8, 4, 5, 6, 7, 8, 9, 10 or more exons of ALK. In certain embodiments, the TPM3-ALK fusion comprises a fusion of exon 8 from TPM3 and exon 20 from ALK. In another embodiment, the TPM3-ALK fusion comprises 1, 2, 3, 4, 5, 6, 7, 8 or more exons of TPM3; and at least 1, 2, 3, 4, 5, 6, 7, 8, 4, 5, 6, 7, 8, 9, 10 or more exons of ALK.

In one embodiment, the nucleic acid molecule includes a nucleotide sequence that has an in-frame fusion of intron 8 of TPM3 (e.g., NM_{—}152263) with intron 19 of ALK (e.g., NM_{—}004304). In another embodiment, the nucleic acid molecule includes a nucleotide sequence that includes a breakpoint. For example, the nucleic acid molecule includes a nucleotide sequence that includes the fusion junction between the TPM3 gene and the ALK gene, e.g., the breakpoint between intron 8 of TPM3 and intron 19 of ALK. In other embodiments, the nucleic acid molecules includes a nucleotide sequence of one or more of nucleotide of chromosome 1 coupled to (e.g., directly or indirectly juxtaposed to) one or more of nucleotide of chromosome 2. In one embodiment, the nucleic acid molecule includes the nucleotide sequence of: chromosome 1 at one or more of a nucleotide plus or minus 10, 20, 30, 40 50, 60, 80, 100, 150 nucleotides and chromosome 2 at one or more of a nucleotide plus or minus 10, 20, 30, 40 50, 60, 80, 100, 150 nucleotides (corresponding to the breakpoint of a TPM3-ALK fusion), or a fragment thereof, or a sequence substantially identical thereto. In one embodiment, the nucleic acid molecule is complementary to at least a portion of a nucleotide sequence disclosed herein, e.g., is capable of hybridizing under a stringency condition described herein to SEQ ID NO:9 and/or SEQ ID NO:7 or a fragment thereof. In yet other embodiment, the nucleic acid molecule hybridizes to a nucleotide sequence that is complementary to at least a portion of a nucleotide sequence disclosed herein, e.g., is capable of hybridizing under a stringency condition described herein to a nucleotide sequence complementary to SEQ ID NO:9 or SEQ ID NO:7 or a fragment thereof.

In another embodiment, the TPM3-ALK fusion nucleic acid comprises at least 6, 12, 15, 20, 25, 50, 75, 100 or more nucleotides from exon 8 of TPM3 (e.g., from the nucleotide sequence of TPM3 preceding the fusion junction with ALK, e.g., of the TPM3 sequence shown in SEQ ID NO:9), and at least 6, 12, 15, 20, 25, 50, 75, 100 or more nucleotides from exon 20 of ALK (e.g., from the nucleotide sequence of ALK following the fusion junction with TPM3, e.g., of the ALK sequence shown in SEQ ID NO:7).

In other embodiments, the nucleic acid molecule includes a nucleotide sequence encoding a TPM3-ALK fusion polypeptide that includes a fragment of a TPM3 gene and a fragment of an ALK gene. In one embodiment, the nucleotide sequence encodes a TPM3-ALK fusion polypeptide that includes e.g., a tyrosine kinase domain or a functional fragment thereof. In yet other embodiments, the nucleic acid molecule includes a nucleotide sequence encoding the amino acid sequence (e.g., a fragment of the amino acid sequence) shown in SEQ ID NO: 10 and a nucleotide sequence encoding the amino acid sequence (e.g., a fragment of the amino acid sequence) shown in SEQ ID NO:7, or a fragment of the fusion, or a sequence substantially identical thereto. In one embodiment, the encoded TPM3-ALK fusion polypeptide includes a tyrosine kinase domain or a functional fragment thereof.

In a related aspect, the invention features nucleic acid constructs that include the TPM3-ALK nucleic acid molecules described herein. In certain embodiments, the nucleic acid molecules are operatively linked to a native or a heterologous regulatory sequence. Also included are vectors and host cells that include the TPM3-ALK nucleic acid molecules described herein, e.g., vectors and host cells suitable for producing the nucleic acid molecules and polypeptides described herein.

In a related aspect, methods of producing the nucleic acid molecules and polypeptides described herein are also described.

In another aspect, the invention features nucleic acid molecules that reduce or inhibit the expression of a nucleic acid molecule that encodes a TPM3-ALK fusion described herein. Examples of such nucleic acid molecules include, for example, antisense molecules, ribozymes, RNAi, triple helix molecules that hybridize to a nucleic acid encoding TPM3-ALK, or a transcription regulatory region of TPM3-ALK, and blocks or reduces mRNA expression of TPM3-ALK.

### Nucleic Acid Detection and Capturing Reagents

The invention also features a nucleic acid molecule, e.g., nucleic acid fragment, suitable as probe, primer, bait or library member that includes, flanks, hybridizes to, which are useful for identifying, or are otherwise based on, the TPM3-ALK fusions described herein. In certain embodiments, the probe, primer or bait molecule is an oligonucleotide that allows capture, detection or isolation of a TPM3-ALK fusion nucleic acid molecule described herein. The oligonucleotide can comprise a nucleotide sequence substantially complementary to a fragment of the TPM3-ALK fusion nucleic acid molecules described herein. The sequence identity between the nucleic acid fragment, e.g., the oligonucleotide, and the target TPM3-ALK sequence need not be exact, so long as the sequences are sufficiently complementary to allow the capture, detection or isolation of the target sequence. In one embodiment, the nucleic acid fragment is a probe or primer that includes an oligonucleotide between about 5 and 25, e.g., between 10 and 20, or 10 and 15 nucleotides in length. In other embodiments, the nucleic acid fragment is a bait that includes an oligonucleotide between about 100 to 300 nucleotides, 130 and 230 nucleotides, or 150 and 200 nucleotides, in length.

In one embodiment, the nucleic acid fragment can be used to identify or capture, e.g., by hybridization, a TPM3-ALK fusion. For example, the nucleic acid fragment can be a probe, a primer, or a bait, for use in identifying or capturing, e.g., by hybridization, a TPM3-ALK fusion described herein. In one embodiment, the nucleic acid fragment can be useful for identifying or capturing a TPM3-ALK breakpoint, e.g., the nucleotide sequence of: chromosome 1 at nucleotide plus or minus 10, 20, 30, 40, 50, 60, 80, 100, 150 nucleotides and chromosome 2 at nucleotide plus or minus 10, 20, 30, 40 50, 60, 80, 100, 150 nucleotides.

In one embodiment, the nucleic acid fragment hybridizes to a nucleotide sequence within a chromosomal rearrangement that creates an in-frame fusion of intron 8 of TPM3 with intron 19 of ALK. In one embodiment, the nucleic acid fragment hybridizes to a nucleotide sequence in the region In other embodiments, the nucleic acid molecules includes a nucleotide sequence in the region of a nucleotide sequence of chromosome 1 coupled to (e.g., juxtaposed to) nucleotides in the region of a nucleotide sequence of chromosome 2. In one embodiment, the nucleic acid fragment hybridizes to a nucleotide sequence that includes a breakpoint, e.g., the nucleotide sequence of: chromosome 1 at nucleotide plus or minus 10, 20, 30, 40 50, 60, 80, 100, 150 or more nucleotides and chromosome 2 at nucleotide plus or minus 10, 20, 30, 40 50, 60, 80, 100, 150 or more nucleotides. For example, the nucleic acid fragment can hybridize to a nucleotide sequence that includes the fusion junction between the TPM3 gene and the ALK gene, e.g., a nucleotide sequence that includes a portion of a nucleotide sequence within intron 8 of a TPM3 gene and intron 19 of an ALK gene.

In another embodiment, the nucleic acid fragment hybridizes to a nucleotide sequence that comprises at least 6, 12, 15, 20, 25, 50, 75, 100, 150 or more nucleotides from exon 8 of TPM3 (e.g., from the nucleotide sequence of TPM3 preceding the fusion junction with ALK, e.g., of the TPM3 sequence shown in SEQ ID NO:9), and at least 6, 12, 15, 20, 25, 50, 75, 100, 150 or more nucleotides from exon 20 of ALK (e.g., from the nucleotide sequence of ALK following the fusion junction with TPM3, e.g., of the ALK sequence shown in SEQ ID NO:7).

The probes or primers described herein can be used, for example, for FISH detection or PCR amplification. In one exemplary embodiment where detection is based on PCR, amplification of the TPM3-ALK fusion junction fusion junction can be performed using a primer or a primer pair, e.g., for amplifying a sequence flanking the fusion junctions described herein, e.g., the mutations or the junction of a chromosomal rearrangement described herein, e.g., TPM3-ALK.

In one embodiment, a pair of isolated oligonucleotide primers can amplify a region containing or adjacent to a position in the TPM3-ALK fusion. For example, forward primers can be designed to hybridize to a nucleotide sequence within TPM3 genomic or mRNA sequence (e.g., a nucleotide sequence within exon 8 of TPM3 of SEQ ID NO:9), and the reverse primers can be designed to hybridize to a nucleotide sequence of ALK (e.g., a nucleotide sequence within exon 20 of ALK, of SEQ ID NO:7).

In another embodiment, the nucleic acid fragments can be used to identify, e.g., by hybridization, a TPM3-ALK fusion. In one embodiment, the nucleic acid fragment hybridizes to a nucleotide sequence that includes a fusion junction between the TPM3 transcript and the ALK transcript.

In other embodiments, the nucleic acid fragment includes a bait that comprises a nucleotide sequence that hybridizes to a TPM3-ALK fusion nucleic acid molecule described herein, and thereby allows the capture or isolation said nucleic acid molecule. In one embodiment, a bait is suitable for solution phase hybridization. In other embodiments, a bait includes a binding entity, e.g., an affinity tag, that allows capture and separation, e.g., by binding to a binding entity, of a hybrid formed by a bait and a nucleic acid hybridized to the bait.

In other embodiments, the nucleic acid fragment includes a library member comprising a TPM3-ALK nucleic acid molecule described herein. In one embodiment, the library member includes a rearrangement that results in a TPM3-ALK fusion described herein.

The nucleic acid fragment can be detectably labeled with, e.g., a radiolabel, a fluorescent label, a bioluminescent label, a chemiluminescent label, an enzyme label, a binding pair label, or can include an affinity tag; a tag, or identifier (e.g., an adaptor, barcode or other sequence identifier).

### TPM3-ALK Fusion Polypeptides

In another embodiment, the TPM3-ALK fusion comprises an amino acid sequence (e.g., a fragment of the amino acid sequence) shown in SEQ ID NO:10 and an amino acid sequence (e.g., a fragment of the amino acid sequence) shown in SEQ ID NO:7, or a fragment of the fusion. In one embodiment, the TPM3-ALK fusion comprises an amino acid sequence substantially identical to the amino acid sequence (e.g., a fragment of the amino acid sequence) shown in SEQ ID NO: 10 and the amino acid sequence (e.g., a fragment of the amino acid sequence) shown in SEQ ID NO:7, or a fragment thereof. In one embodiment, the TPM3-ALK fusion comprises an amino acid sequence at least 70%, at least 75%, at least 80%, at least 85%, at least 90%, at least 95%, at least 99%, at least 99.5 or greater, identical to the amino acid sequence (e.g., a fragment of the amino acid sequence) shown in SEQ ID NO:10 and the amino acid sequence (e.g., a fragment of the amino acid sequence) shown in SEQ ID NO:7. In one embodiment, the TPM3-ALK fusion comprises a sequence containing at least 10, 20, 50, 100, 500, 600, 700, 800, 900, 1000, or more amino acids of the amino acid sequence shown in SEQ ID NO: 10 and SEQ ID NO:7. In one embodiment, the TPM3-ALK fusion comprises an amino acid sequence containing at least 5, 10, 20, 50, 100, 500, 600, 700, 800, 900, 1000, or more contiguous amino acids of the amino acid sequence shown in SEQ ID NO:10 and at least 5, 10, 20, 50, 100, 500, 600, 700, 800, 900, 1000, or more contiguous amino acids of the amino acid sequence shown in SEQ ID NO:7. In one embodiment, the 5′ TPM3-3′ ALK fusion polypeptide includes a receptor tyrosine kinase domain or a functional fragment thereof. In an embodiment, the 5′TPM3-3′ALK fusion polypeptide comprises sufficient ALK and sufficient TPM3 sequence such that it has kinase activity, e.g., has elevated activity.

In another aspect, the invention features a TPM3-ALK fusion polypeptide (e.g., a purified TPM3-ALK fusion polypeptide), a biologically active or antigenic fragment thereof, as well as reagents (e.g., antibody molecules that bind to a TPM3-ALK fusion polypeptide), methods for modulating a TPM3-ALK polypeptide activity and detection of a TPM3-ALK polypeptide.

In one embodiment, the TPM3-ALK fusion polypeptide has at least one biological activity. In one embodiment, at least one biological activity of the TPM3-ALK fusion polypeptide is reduced or inhibited by an anti-cancer drug, e.g., a TPM3 inhibitor, an ALK inhibitor. In one embodiment, at least one biological activity of the TPM3-ALK fusion polypeptide is reduced or inhibited by an ALK inhibitor. In one embodiment, at least one biological activity of the TPM3-ALK fusion polypeptide is reduced or inhibited by a TPM3 inhibitor. In one embodiment, at least one biological activity of the TPM3-ALK fusion polypeptide is reduced or inhibited by an ALK inhibitor, e.g., TAE-684 (also referred to herein as “NVP-TAE694”), PF02341066 (also referred to herein as “crizotinib” or “1066”), AF-802, LDK-378, ASP-3026, CEP-37440, CEP-28122, CEP-108050. MK-2206, perifosine, sorafenib and AP26113; and additional examples of ALK kinase inhibitors are described in examples 3-39 of WO 2005016894 by Garcia-Echeverria C, et al.

In yet other embodiments, the TPM3-ALK fusion polypeptide is encoded by a nucleic acid molecule described herein. In one embodiment, the TPM3-ALK fusion polypeptide is encoded by an in-frame fusion of intron 8 of TPM3 with intron 19 of ALK (e.g., a sequence on chromosome 1 and a sequence on chromosome 2. In another embodiment, the TPM3-ALK fusion polypeptide includes an amino acid sequence encoded by a nucleotide sequence comprising a fusion junction between the TPM3 transcript and the ALK transcript.

In certain embodiments, the TPM3-ALK fusion polypeptide comprises one or more of encoded exons 1-8 from TPM3 and one or more of encoded exons 20-29 of ALK. In certain embodiments, the TPM3-ALK fusion polypeptide comprises at least 1, 2, 3, 4, 5, 6, 7, 8 or more encoded exons of TPM3 and at least at least 1, 2, 3, 4, 5, 6, 7, 8, 4, 5, 6, 7, 8, 9, 10 or more encoded exons of ALK. In certain embodiments, the TPM3-ALK fusion polypeptide comprises a fusion of encoded exon 8 from TPM3 and encoded exon 20 from ALK (or a fragment thereof). In other embodiments, the fusion comprises at least 1, 2, 3, 4, 5, 6, 7, 8 or more encoded exons of TPM3; and at least 1, 2, 3, 4, 5, 6, 7, 8, 4, 5, 6, 7, 8, 9, 10 or more encoded exons of ALK. In certain embodiments, the TPM3-ALK fusion polypeptide comprises encoded exons 1-8 from TPM3 and exons 20-29 of ALK. In certain embodiments, the 5′ TPM3-3′ ALK fusion polypeptide comprises a fusion junction of the sequence of exon 8 from TPM3 and the sequence of exon 20 from ALK.

In certain embodiments, the TPM3-ALK fusion comprises the amino acid sequence corresponding to exon 8 or a fragment thereof from TPM3, and the amino acid sequence corresponding to exon 20 or a fragment thereof from ALK (e.g., as shown in SEQ ID NO:10 and SEQ ID NO:7). In one embodiment, the TPM3-ALK fusion comprises at least 5, 10, 15, 20 or more amino acids from exon 8 of TPM3 (e.g., from the amino acid sequence of TPM3 preceding the fusion junction with ALK, e.g., of the TPM3 sequence shown in SEQ ID NO:10), and at least 5, 10, 15, 20 or more amino acids from exon 20 of ALK (e.g., from the amino acid sequence of ALK following the fusion junction with TPM3, e.g., of the ALK sequence shown in SEQ ID NO:7).

In one embodiment, the TPM3-ALK fusion polypeptide includes a tyrosine kinase domain or a functional fragment thereof. In a related aspect, the invention features TPM3-ALK fusion polypeptide or fragments operatively linked to heterologous polypeptides to form fusion proteins.

In another embodiment, the TPM3-ALK fusion polypeptide or fragment is a peptide, e.g., an immunogenic peptide or protein that contains a fusion junction described herein. Such immunogenic peptides or proteins can be used to raise antibodies specific to the fusion protein. In other embodiments, such immunogenic peptides or proteins can be used for vaccine preparation. The vaccine preparation can include other components, e.g., an adjuvant.

In another aspect, the invention features antibody molecules that bind to a TPM3-ALK fusion polypeptide or fragment described herein. In embodiments, the antibody can distinguish wild type ALK (or TPM3) from TPM3-ALK.

### Detection Reagents and Detection of Mutations

In another aspect, the invention features a detection reagent, e.g., a purified or an isolated preparation thereof. Detection reagents can distinguish a nucleic acid, or protein sequence, having a breakpoint, e.g., a TPM3-ALK breakpoint; from a reference sequence. In one embodiment, the detection reagent detects (e.g., specifically detects) a TPM3-ALK fusion nucleic acid or a polypeptide, e.g., distinguishes a wild type ALK or another ALK fusion (or TPM3) from a TPM3-ALK nucleic acid (e.g., as described herein in SEQ ID NO:9 and SEQ ID NO:7); or a TPM3-ALK polypeptide (e.g., as described herein in SEQ ID NO: 10 and SEQ ID NO:7).

Detection reagents, e.g., nucleic acid-based detection reagents, can be used to identify mutations in a target nucleic acid, e.g., DNA, e.g., genomic DNA or cDNA, or RNA, e.g., in a sample, e.g., a sample of nucleic acid derived from a neoplastic or tumor cell, e.g., a melanocytic neoplasm, melanoma or metastatic cell. Detection reagents, e.g., antibody-based detection reagents, can be used to identify mutations in a target protein, e.g., in a sample, e.g., a sample of protein derived from, or produced by, a neoplastic or tumor cell, e.g., a melanocytic neoplasm, melanoma or metastatic cell.

### Method of Treatment

In a related aspect, a method of inhibiting, reducing, or treating a hyperproliferative disorder, e.g., a neoplasm (including benign, pre-malignant or malignant (e.g., a cancer), in a subject is provided. The method includes administering to the subject a preselected therapeutic agent, e.g., an anti-cancer agent (e.g., a kinase inhibitor), as a single agent, or in combination, in an amount sufficient to reduce, inhibit or treat the activity or expression of TPM3-ALK (e.g., a TPM3-ALK fusion described herein), thereby inhibiting, reducing, or treating the hyperproliferative disorder in the subject.

In one embodiment, the subject treated has a TPM3-ALK fusion; e.g., the subject has a tumor or cancer harboring a TPM3-ALK fusion. In other embodiments, the subject has been previously identified as having a TPM3-ALK fusion. In yet other embodiments, the subject has been previously identified as being likely or unlikely to respond to treatment with a protein kinase inhibitor, e.g., a subject that has previously participated in a clinical trial. In other embodiments, the subject has been previously identified as being likely or unlikely to respond to treatment with a protein kinase inhibitor, based on the presence of the TPM3-ALK fusion. In one embodiment, the subject is a mammal, e.g., a human. In one embodiment, the subject has, or at risk of having a cancer at any stage of disease. In other embodiments, the subject is a patient, e.g., a cancer patient.

In certain embodiments, the neoplasm or neoplastic cell is a benign, pre-malignant, malignant (cancer) or metastasis. In certain embodiments, the cancer is a solid tumor, a soft tissue tumor, or a metastatic lesion.

In one embodiment, the neoplasm or neoplastic cell is a benign, pre-malignant, malignant (cancer) or metastasis. In certain embodiments, the cancer is a solid tumor, a soft tissue tumor, or a metastatic lesion. In one embodiment, the cancer is a carcinoma. In one embodiment, the cancer is an adenocarcinoma. In an embodiment, the cancer is a lung adenocarcinoma. In one embodiment, the cancer is chosen from a lung cancer, a pancreatic cancer, melanoma, a colorectal cancer, an esophageal-gastric cancer, a thyroid cancer, or an adenocarcinoma. In one embodiment, the cancer is a lymphoma. In one embodiment, the cancer is an anaplastic large cell lymphoma. In one embodiment, the cancer is an inflammatory myofibrotic tumor. In one embodiment, the cancer is a lung adenocarcinoma. In other embodiment, the lung cancer is chosen from one or more of the following: non-small cell lung cancer (NSCLC), small cell lung cancer (SCLC), squamous cell carcinoma (SCC), adenocarcinoma of the lung, bronchogenic carcinoma, or a combination thereof. In one embodiment, the lung cancer is NSCLC or SCC.

In one embodiment, the anti-cancer agent is an ALK inhibitor. In one embodiment, the anti-cancer agent is a TPM3 inhibitor. In one embodiment, the anti-cancer agent is a kinase inhibitor. In one embodiment, the anti-cancer agent is an ALK inhibitor, e.g., TAE-684 (also referred to herein as “NVP-TAE694”), PF02341066 (also referred to herein as “crizotinib” or “1066”), AF-802, LDK-378, ASP-3026, CEP-37440, CEP-28122, CEP-108050, MK-2206, perifosine, sorafenib and AP26113; and additional examples of ALK kinase inhibitors are described in examples 3-39 of WO 2005016894 by Garcia-Echeverria C, et al.

### GOLGA5-RET Fusions

In one embodiment, a fusion includes an in-frame fusion of an exon of golgin A5 (GOLGA5), e.g., one more exons of GOLGA5 (e.g., one or more of exons 1-7 of GOLGA5) or a fragment thereof, and an exon of ret proto-oncogene (RET), e.g., one or more exons of a RET (e.g., one or more of exons 12-19 of RET) or a fragment thereof. For example, the GOLGA5-RET fusion can include an in-frame fusion within an intron of GOLGA5 (e.g., intron 7) or a fragment thereof, with an intron of RET (e.g., intron 1) or a fragment thereof. In one embodiment, the fusion of the GOLGA5-RET fusion comprises the nucleotide sequence of: chromosome 14 at one or more of a nucleotide (plus or minus 10, 20, 30, 50, 60, 70, 80, 100 or more nucleotides) and chromosome 10 at one or more of a nucleotide (plus or minus 10, 20, 30, 50, 60, 70, 80, 100 or more nucleotides). In one embodiment, the GOLGA5-RET fusion is a translocation, e.g., a translocation of a portion of chromosome 14 and a portion of chromosome 10.

In certain embodiments, the GOLGA5-RET fusion is in a 5′-GOLGA5 to 3′-RET configuration (also referred to herein as “5′-GOLGA5-RET-3′).” The term “fusion” or “fusion molecule” can refer to a polypeptide or a nucleic acid fusion, depending on the context. It may include a full-length sequence of a fusion or a fragment thereof, e.g., a fusion junction (e.g., a fragment including a portion of GOLGA5 and a portion of RET, e.g., a portion of the GOLGA5-RET fusion described herein). In one embodiment, the GOLGA5-RET fusion polypeptide includes a fragment of the amino acid sequence shown in SEQ ID NO:48 and a fragment of the amino acid sequence shown in SEQ ID NO:6, or an amino acid sequence substantially identical thereto. In another embodiment, the GOLGA5-RET fusion nucleic acid includes a fragment of the nucleotide sequence shown in SEQ ID NO:47 and a fragment of the nucleotide sequence shown in SEQ ID NO:5, or a nucleotide sequence substantially identical thereto. In one embodiment, the GOLGA5-RET fusion polypeptide comprises sufficient GOLGA5 and sufficient RET sequence such that the 5′ GOLGA5-3′ RET fusion has kinase activity, e.g., has elevated activity, e.g., tyrosine kinase activity. In any event, the fusion causes activation in the cells harboring it of oncogenic signaling pathways.

In certain embodiments, the GOLGA5-RET fusion comprises one or more (or all of) exons 1-7 from GOLGA5 and one or more (or all of) exons 12-19 of RET (e.g., one or more of the exons shown in SEQ ID NO:47 and SEQ ID NO:5. In another embodiment, the GOLGA5-RET fusion comprises one or more (or all of) exons 1-7 of GOLGA5 and one or more (or all of) exons 12-19 of RET. In certain embodiments, the GOLGA5-RET fusion comprises at least 1, 2, 3, 4, 5, 6, 7 or more exons (or encoded exons) from GOLGA5 and at least 1, 2, 3, 4, 5, 6, 7, 8 or more exons (or encoded exons) from RET (e.g., from the GOLGA5 and RET sequences shown in SEQ ID NO:47 and SEQ ID NO:48 and SEQ ID NO:5 and SEQ ID NO:6.

In certain embodiments, the GOLGA5-RET fusion comprises exons 1-7 or a fragment thereof from GOLGA5, and exons 12-19 or a fragment thereof from RET (e.g., as shown in SEQ ID NO:47 and SEQ ID NO:5). In one embodiment, the GOLGA5-RET fusion comprises at least 5, 10, 15, 20, 30, 40, 50 or more amino acids from exons 1-7 of GOLGA5 (e.g., from the amino acid sequence of GOLGA5 as shown in SEQ ID NO:48 (e.g., from the amino acid sequence of GOLGA5 preceding the fusion junction with RET, and at least 5, 10, 15, 20, 30, 40, 50 or more amino acids from exons 12-19 of RET (e.g., from the amino acid sequence of RET as shown in SEQ ID NO:6). In another embodiment, the GOLGA5-RET fusion comprises at least 6, 12, 15, 20, 25, 50, 75, 100 or more nucleotides from exons 1-7 of GOLGA5 (e.g., from the nucleotide sequence of GOLGA5 as shown in SEQ ID NO:47 (e.g., from the nucleotide sequence of GOLGA5 preceding the fusion junction with RET); and at least 6, 12, 15, 20, 25, 50, 75, 100 or more nucleotides from exons 12-19 of RET (e.g., from the nucleotide sequence of RET as shown in SEQ ID NO:5).

In one embodiment, the GOLGA5-RET fusion includes the full sequence or a fragment of the nucleotide sequence shown in SEQ ID NO:45 and SEQ ID NO:102, or a nucleotide sequence substantially identical thereto. In another embodiment, the GOLGA5-RET fusion polypeptide includes the full sequence or a fragment of the amino acid sequence shown in SEQ ID NO:46 and SEQ ID NO: 103, or an amino acid sequence substantially identical thereto, for example, at least 70% or at least 80% or at least 90% identical or even more.

### GOLGA5-RET Nucleic Acid Molecules

In one aspect, the invention features a nucleic acid molecule (e.g., an isolated or purified) nucleic acid molecule that includes a fragment of a GOLGA5 gene and a fragment of a RET gene. In one embodiment, the nucleotide sequence encodes a GOLGA5-RET fusion polypeptide that includes a tyrosine kinase domain or a functional fragment thereof. In another embodiment, the nucleotide sequence encodes a fragment of the RET polypeptide including the amino acid sequence of SEQ ID NO:6 or a fragment thereof, or a sequence substantially identical thereto. In other embodiments, the nucleic acid molecule includes a fragment of the GOLGA5 gene encoding the amino acid sequence of SEQ ID NO:48 or a fragment thereof, or a sequence substantially identical thereto. In yet other embodiments, the nucleic acid molecule includes a nucleotide sequence encoding the amino acid sequence shown in SEQ ID NO:48, or a fragment thereof, and the amino acid sequence shown in SEQ ID NO:6 or a fragment thereof, or a sequence substantially identical thereto.

In one embodiment, the nucleic acid molecule includes a fusion. e.g., an in-frame fusion, between an intron of GOLGA5 (e.g., intron 7, or a fragment thereof), and an intron of RET (e.g., intron 11, or a fragment thereof). The GOLGA5-RET fusion can comprise a fusion of the nucleotide sequence of: chromosome 14 at one or more of a nucleotide (plus or minus 10, 20, 30, 50, 60, 70, 80, 100 nucleotides) and chromosome 10 at one or more of a nucleotide (plus or minus 10, 20, 30, 50, 60, 70, 80, 100 nucleotides), or a fragment thereof. In one embodiment, the GOLGA5-RET fusion comprises a fusion of the nucleotide sequence of: chromosome 14 at one or more of a nucleotide (plus or minus 10, 20, 30, 50, 60, 70, 80, 100 nucleotides) and chromosome 10 at one or more of a nucleotide (plus or minus 10, 20, 30, 50, 60, 70, 80, 100 nucleotides), or a fragment thereof.

In another embodiment, the GOLGA5-RET fusion comprises a nucleotide sequence (e.g., a fragment of a nucleotide sequence) shown in SEQ ID NO:47 and a nucleotide sequence (e.g., a fragment of a nucleotide sequence) shown in SEQ ID NO:5, or a fragment of the fusion. In one embodiment, the GOLGA5-RET fusion comprises a nucleotide sequence substantially identical to the nucleotide sequence (e.g., a fragment of a nucleotide sequence) shown in SEQ ID NO:47 and the nucleotide sequence (e.g., a fragment of a nucleotide sequence) shown SEQ ID NO:5, or a fragment of the fusion. In one embodiment, the GOLGA5-RET fusion comprises a nucleotide sequence at least 70%, at least 75%, at least 80%, at least 85%, at least 90%, at least 95%, at least 99%, at least 99.5 or greater, identical to the nucleotide sequence (e.g., a fragment of a nucleotide sequence) shown in SEQ ID NO:47 and to the nucleotide sequence (e.g., a fragment of a nucleotide sequence) shown in SEQ ID NO:5. In one embodiment, the GOLGA5-RET fusion comprises a nucleotide sequence containing at least 25, 50, 100, 150, 200, 500, 1000, 1500, 2000, 2500, 3000, or more nucleotides of the nucleotide sequence shown in SEQ ID NO:47 and a nucleotide sequence containing at least 25, 50, 100, 150, 200, 500, 1000, 1500, 2000, 2500, 3000, or more nucleotides of the nucleotide sequence shown in SEQ ID NO:5. In one embodiment, the GOLGA5-RET fusion comprises a nucleotide sequence containing at least 25, 50, 100, 150, 200, 500, 1000, 1500, 2000, 2500, 3000, or more contiguous nucleotides of the nucleotide sequence shown in SEQ ID NO:47 and a nucleotide sequence containing at least 25, 50, 100, 150, 200, 500, 1000, 1500, 2000, 2500, 3000, or more nucleotides of the nucleotide sequence shown in SEQ ID NO:5.

In another embodiment, the nucleic acid molecule includes a fusion, e.g., an in-frame fusion, of at least 1, 2, 3, 4, 5, 6, 7 or more exons of GOLGA5 or a fragment thereof (e.g., one or more of exons 1-7 of GOLGA5 or a fragment thereof), and at least 1, 2, 3, 4, 5, 6, 7, 8 or more exons of RET or a fragment thereof (e.g., one or more of exons 12-19 of RET or a fragment thereof). In yet other embodiments, the nucleic acid molecule includes a fragment the nucleotide sequence shown in SEQ ID NO:47 and a fragment of the nucleotide sequence shown in SEQ ID NO:5 or a fragment of the fusion, or a sequence substantially identical thereto.

In one embodiment, the nucleic acid molecule is complementary to at least a portion of a nucleotide sequence disclosed herein, e.g., is capable of hybridizing under a stringency condition described herein to SEQ ID NO:47 and/or SEQ ID NO:5, or a fragment thereof. In yet another embodiment, the nucleic acid molecule hybridizes to a nucleotide sequence that is complementary to at least a portion of a nucleotide sequence disclosed herein, e.g., is capable of hybridizing under a stringency condition to a nucleotide sequence complementary to SEQ ID NO:47 and/or SEQ ID NO:5, or a fragment thereof. The nucleotide sequence of a cDNA encoding an exemplary 5′ GOLGA5-3′ RET fusion is shown in at least exon 7 (e.g., exons 1-7) of SEQ ID NO:47 and at least exon 12 (e.g., exons 12-19) of SEQ ID NO:5, and the predicted amino acid sequence is shown in the corresponding encoded exons of SEQ ID NO:48 and the corresponding encoded exons of SEQ ID NO:6, respectively.

In an embodiment the GOLGA5-RET nucleic acid molecule comprises sufficient GOLGA5 and sufficient RET sequence such that the encoded 5′ GOLGA5-3′ RET fusion has kinase activity, e.g., has elevated activity. In certain embodiments, the 5′ GOLGA5-3′ RET fusion comprises exons 1-7 from GOLGA5 and exons 12-19 from RET. In certain embodiments, the GOLGA5-RET fusion comprises at least 1, 2, 3, 4, 5, 6, 7 or more exons from GOLGA5 and at least at least 1, 2, 3, 4, 5, 6, 7, 8 or more exons of RET. In certain embodiments, the GOLGA5-RET fusion comprises a fusion of exon 7 from GOLGA5 and exon 12 from RET. In another embodiment, the GOLGA5-RET fusion comprises 1, 2, 3, 4, 5, 6, 7 or more exons of GOLGA5; and at least 1, 2, 3, 4, 5, 6, 7, 8 or more exons of RET.

In one embodiment, the nucleic acid molecule includes a nucleotide sequence that has an in-frame fusion of intron 7 of GOLGA5 (e.g., NM_{—}005113) with intron 11 of RET (e.g., NM_{—}020630). In another embodiment, the nucleic acid molecule includes a nucleotide sequence that includes a breakpoint. For example, the nucleic acid molecule includes a nucleotide sequence that includes the fusion junction between the GOLGA5 gene and the RET gene, e.g., the breakpoint between intron 7 of GOLGA5 and intron 11 of RET. In other embodiments, the nucleic acid molecules includes a nucleotide sequence of one or more of nucleotide of chromosome 14 coupled to (e.g., directly or indirectly juxtaposed to) one or more of nucleotide of chromosome 10. In one embodiment, the nucleic acid molecule includes the nucleotide sequence of: chromosome 14 at one or more of a nucleotide plus or minus 10, 20, 30, 40 50, 60, 80, 100, 150 nucleotides and chromosome 10 at one or more of a nucleotide plus or minus 10, 20, 30, 40 50, 60, 80, 100, 150 nucleotides (corresponding to the breakpoint of a GOLGA5-RET fusion), or a fragment thereof, or a sequence substantially identical thereto. In one embodiment, the nucleic acid molecule is complementary to at least a portion of a nucleotide sequence disclosed herein, e.g., is capable of hybridizing under a stringency condition described herein to SEQ ID NO:47 and/or SEQ ID NO:5 or a fragment thereof. In yet other embodiment, the nucleic acid molecule hybridizes to a nucleotide sequence that is complementary to at least a portion of a nucleotide sequence disclosed herein, e.g., is capable of hybridizing under a stringency condition described herein to a nucleotide sequence complementary to SEQ ID NO:47 or SEQ ID NO:5 or a fragment thereof.

In another embodiment, the GOLGA5-RET fusion nucleic acid comprises at least 6, 12, 15, 20, 25, 50, 75, 100 or more nucleotides from exon 7 of GOLGA5 (e.g., from the nucleotide sequence of GOLGA5 preceding the fusion junction with RET, e.g., of the GOLGA5 sequence shown in SEQ ID NO:47), and at least 6, 12, 15, 20, 25, 50, 75, 100 or more nucleotides from exon 12 of RET (e.g., from the nucleotide sequence of RET following the fusion junction with GOLGA5, e.g., of the RET sequence shown in SEQ ID NO:5).

In other embodiments, the nucleic acid molecule includes a nucleotide sequence encoding a GOLGA5-RET fusion polypeptide that includes a fragment of a GOLGA5 gene and a fragment of a RET gene. In one embodiment, the nucleotide sequence encodes a GOLGA5-RET fusion polypeptide that includes e.g., a tyrosine kinase domain or a functional fragment thereof. In yet other embodiments, the nucleic acid molecule includes a nucleotide sequence encoding the amino acid sequence (e.g., a fragment of the amino acid sequence) shown in SEQ ID NO:48 and a nucleotide sequence encoding the amino acid sequence (e.g., a fragment of the amino acid sequence) shown in SEQ ID NO:6, or a fragment of the fusion, or a sequence substantially identical thereto. In one embodiment, the encoded GOLGA5-RET fusion polypeptide includes a tyrosine kinase domain or a functional fragment thereof.

In a related aspect, the invention features nucleic acid constructs that include the GOLGA5-RET nucleic acid molecules described herein. In certain embodiments, the nucleic acid molecules are operatively linked to a native or a heterologous regulatory sequence. Also included are vectors and host cells that include the GOLGA5-RET nucleic acid molecules described herein. e.g., vectors and host cells suitable for producing the nucleic acid molecules and polypeptides described herein.

In a related aspect, methods of producing the nucleic acid molecules and polypeptides described herein are also described.

In another aspect, the invention features nucleic acid molecules that reduce or inhibit the expression of a nucleic acid molecule that encodes a GOLGA5-RET fusion described herein. Examples of such nucleic acid molecules include, for example, antisense molecules, ribozymes, RNAi, triple helix molecules that hybridize to a nucleic acid encoding GOLGA5-RET, or a transcription regulatory region of GOLGA5-RET, and blocks or reduces mRNA expression of GOLGA5-RET.

### Nucleic Acid Detection and Capturing Reagents

The invention also features a nucleic acid molecule, e.g., nucleic acid fragment, suitable as probe, primer, bait or library member that includes, flanks, hybridizes to, which are useful for identifying, or are otherwise based on, the GOLGA5-RET fusions described herein. In certain embodiments, the probe, primer or bait molecule is an oligonucleotide that allows capture, detection or isolation of a GOLGA5-RET fusion nucleic acid molecule described herein. The oligonucleotide can comprise a nucleotide sequence substantially complementary to a fragment of the GOLGA5-RET fusion nucleic acid molecules described herein. The sequence identity between the nucleic acid fragment, e.g., the oligonucleotide, and the target GOLGA5-RET sequence need not be exact, so long as the sequences are sufficiently complementary to allow the capture, detection or isolation of the target sequence. In one embodiment, the nucleic acid fragment is a probe or primer that includes an oligonucleotide between about 5 and 25, e.g., between 10 and 20, or 10 and 15 nucleotides in length. In other embodiments, the nucleic acid fragment is a bait that includes an oligonucleotide between about 100 to 300 nucleotides, 130 and 230 nucleotides, or 150 and 200 nucleotides, in length.

In one embodiment, the nucleic acid fragment can be used to identify or capture, e.g., by hybridization, a GOLGA5-RET fusion. For example, the nucleic acid fragment can be a probe, a primer, or a bait, for use in identifying or capturing, e.g., by hybridization, a GOLGA5-RET fusion described herein. In one embodiment, the nucleic acid fragment can be useful for identifying or capturing a GOLGA5-RET breakpoint, e.g., the nucleotide sequence of: chromosome 14 at nucleotide plus or minus 10, 20, 30, 40, 50, 60, 80, 100, 150 nucleotides and chromosome 10 at nucleotide plus or minus 10, 20, 30, 40 50, 60, 80, 100, 150 nucleotides.

In one embodiment, the nucleic acid fragment hybridizes to a nucleotide sequence within a chromosomal rearrangement that creates an in-frame fusion of intron 7 of GOLGA5 with intron 11 of RPET. In one embodiment, the nucleic acid fragment hybridizes to a nucleotide sequence in the region In other embodiments, the nucleic acid molecules includes a nucleotide sequence in the region of a nucleotide sequence of chromosome 14 coupled to (e.g., juxtaposed to) nucleotides in the region of a nucleotide sequence Y of chromosome 10. In one embodiment, the nucleic acid fragment hybridizes to a nucleotide sequence that includes a breakpoint, e.g., the nucleotide sequence of: chromosome 14 at nucleotide plus or minus 10, 20, 30, 40 50, 60, 80, 100, 150 or more nucleotides and chromosome 10 at nucleotide plus or minus 10, 20, 30, 40 50, 60, 80, 100, 150 or more nucleotides. For example, the nucleic acid fragment can hybridize to a nucleotide sequence that includes the fusion junction between the GOLGA5 gene and the RET gene, e.g., a nucleotide sequence that includes a portion of a nucleotide sequence within intron 7 of a GOLGA5 gene and intron 11 of a RET gene.

In another embodiment, the nucleic acid fragment hybridizes to a nucleotide sequence that comprises at least 6, 12, 15, 20, 25, 50, 75, 100, 150 or more nucleotides from exon 7 of GOLGA5 (e.g., from the nucleotide sequence of GOLGA5 preceding the fusion junction with RET, e.g., of the GOLGA5 sequence shown in SEQ ID NO:47), and at least 6, 12, 15, 20, 25, 50, 75, 100, 150 or more nucleotides from exon 12 of RET (e.g., from the nucleotide sequence of RET following the fusion junction with GOLGA5, e.g., of the RET sequence shown in SEQ ID NO:5).

The probes or primers described herein can be used, for example, for FISH detection or PCR amplification. In one exemplary embodiment where detection is based on PCR, amplification of the GOLGA5-RET fusion junction fusion junction can be performed using a primer or a primer pair, e.g., for amplifying a sequence flanking the fusion junctions described herein, e.g., the mutations or the junction of a chromosomal rearrangement described herein, e.g., GOLGA5-RET.

In one embodiment, a pair of isolated oligonucleotide primers can amplify a region containing or adjacent to a position in the GOLGA5-RET fusion. For example, forward primers can be designed to hybridize to a nucleotide sequence within GOLGA5 genomic or mRNA sequence (e.g., a nucleotide sequence within exon 7 of GOLGA5 of SEQ ID NO:47), and the reverse primers can be designed to hybridize to a nucleotide sequence of RET (e.g., a nucleotide sequence within exon 12 of RET, of SEQ ID NO:5).

In another embodiment, the nucleic acid fragments can be used to identify, e.g., by hybridization, a GOLGA5-RET fusion. In one embodiment, the nucleic acid fragment hybridizes to a nucleotide sequence that includes a fusion junction between the GOLGA5 transcript and the RET transcript.

In other embodiments, the nucleic acid fragment includes a bait that comprises a nucleotide sequence that hybridizes to a GOLGA5-RET fusion nucleic acid molecule described herein, and thereby allows the capture or isolation said nucleic acid molecule. In one embodiment, a bait is suitable for solution phase hybridization. In other embodiments, a bait includes a binding entity, e.g., an affinity tag, that allows capture and separation, e.g., by binding to a binding entity, of a hybrid formed by a bait and a nucleic acid hybridized to the bait.

In other embodiments, the nucleic acid fragment includes a library member comprising a GOLGA5-RET nucleic acid molecule described herein. In one embodiment, the library member includes a rearrangement that results in a GOLGA5-RET fusion described herein.

The nucleic acid fragment can be detectably labeled with, e.g., a radiolabel, a fluorescent label, a bioluminescent label, a chemiluminescent label, an enzyme label, a binding pair label, or can include an affinity tag; a tag, or identifier (e.g., an adaptor, barcode or other sequence identifier).

### GOLGA5-RET Fusion Polypeptides

In another embodiment, the GOLGA5-RET fusion comprises an amino acid sequence (e.g., a fragment of the amino acid sequence) shown in SEQ ID NO:48 and an amino acid sequence (e.g., a fragment of the amino acid sequence) shown in SEQ ID NO:6, or a fragment of the fusion. In one embodiment, the GOLGA5-RET fusion comprises an amino acid sequence substantially identical to the amino acid sequence (e.g., a fragment of the amino acid sequence) shown in SEQ ID NO:48 and the amino acid sequence (e.g., a fragment of the amino acid sequence) shown in SEQ ID NO:6, or a fragment thereof. In one embodiment, the GOLGA5-RET fusion comprises an amino acid sequence at least 70%, at least 75%, at least 80%, at least 85%, at least 90%, at least 95%, at least 99%, at least 99.5 or greater, identical to the amino acid sequence (e.g., a fragment of the amino acid sequence) shown in SEQ ID NO:48 and the amino acid sequence (e.g., a fragment of the amino acid sequence) shown in SEQ ID NO:6. In one embodiment, the GOLGA5-RET fusion comprises a sequence containing at least 10, 20, 50, 100, 500, 600, 700, 800, 900, 1000, or more amino acids of the amino acid sequence shown in SEQ ID NO:48 and SEQ ID NO:6. In one embodiment, the GOLGA5-RET fusion comprises an amino acid sequence containing at least 5, 10, 20, 50, 100, 500, 600, 700, 800, 900, 1000, or more contiguous amino acids of the amino acid sequence shown in SEQ ID NO:48 and at least 5, 10, 20, 50, 100, 500, 600, 700, 800, 900, 1000, or more contiguous amino acids of the amino acid sequence shown in SEQ ID NO:6. In one embodiment, the 5′ GOLGA5-3′ RET fusion polypeptide includes a receptor tyrosine kinase domain or a functional fragment thereof. In an embodiment, the 5′GOLGA5-3′RET fusion polypeptide comprises sufficient RET and sufficient GOLGA5 sequence such that it has kinase activity, e.g., has elevated activity.

In another aspect, the invention features a GOLGA5-RET fusion polypeptide (e.g., a purified GOLGA5-RET fusion polypeptide), a biologically active or antigenic fragment thereof, as well as reagents (e.g., antibody molecules that bind to a GOLGA5-RET fusion polypeptide), methods for modulating a GOLGA5-RET polypeptide activity and detection of a GOLGA5-RET polypeptide.

In one embodiment, the GOLGA5-RET fusion polypeptide has at least one biological activity. In one embodiment, at least one biological activity of the GOLGA5-RET fusion polypeptide is reduced or inhibited by an anti-cancer drug, e.g., a GOLGA5 inhibitor, a RET inhibitor. In one embodiment, at least one biological activity of the GOLGA5-RET fusion polypeptide is reduced or inhibited by a RET inhibitor. In one embodiment, at least one biological activity of the GOLGA5-RET fusion polypeptide is reduced or inhibited by a GOLGA5 inhibitor. In one embodiment, at least one biological activity of the GOLGA5-RET fusion polypeptide is reduced or inhibited by a RET inhibitor, e.g., CEP-701 and CEP-751; 2-indolinone, e.g., RPI-1; and quinazoline, e.g., ZD6474; or TG101209.

In yet other embodiments, the GOLGA5-RET fusion polypeptide is encoded by a nucleic acid molecule described herein. In one embodiment, the GOLGA5-RET fusion polypeptide is encoded by an in-frame fusion of intron 7 of GOLGA5 with intron 11 of RET (e.g., a sequence on chromosome 14 and a sequence on chromosome 10). In another embodiment, the GOLGA5-RET fusion polypeptide includes an amino acid sequence encoded by a nucleotide sequence comprising a fusion junction between the GOLGA5 transcript and the RET transcript.

In certain embodiments, the GOLGA5-RET fusion polypeptide comprises one or more of encoded exons 1-7 from GOLGA5 and one or more of encoded exons 12-19 of RET. In certain embodiments, the GOLGA5-RET fusion polypeptide comprises at least 1, 2, 3, 4, 5, 6, 7 or more encoded exons of GOLGA5 and at least at least 1, 2, 3, 4, 5, 6, 7, 8 or more encoded exons of RET. In certain embodiments, the GOLGA5-RET fusion polypeptide comprises a fusion of encoded exon 7 from GOLGA5 and encoded exon 12 from RET (or a fragment thereof). In other embodiments, the fusion comprises at least 1, 2, 3, 4, 5, 6, 7 or more encoded exons of GOLGA5; and at least 1, 2, 3, 4, 5, 6, 7, 8 or more encoded exons of RET. In certain embodiments, the GOLGA5-RET fusion polypeptide comprises encoded exons 1-7 from GOLGA5 and exons 12-19 of RET. In certain embodiments, the 5′ GOLGA5-3′ RET fusion polypeptide comprises a fusion junction of the sequence of exon 7 from GOLGA5 and the sequence of exon 12 from RET.

In certain embodiments, the GOLGA5-RET fusion comprises the amino acid sequence corresponding to exon 7 or a fragment thereof from GOLGA5, and the amino acid sequence corresponding to exon 12 or a fragment thereof from RET (e.g., as shown in SEQ ID NO:48 and 30 SEQ ID NO:6). In one embodiment, the GOLGA5-RET fusion comprises at least 5, 10, 15, 20 or more amino acids from exon 7 of GOLGA5 (e.g., from the amino acid sequence of GOLGA5 preceding the fusion junction with RET, e.g., of the GOLGA5 sequence shown in SEQ ID NO:48), and at least 5, 10, 15, 20 or more amino acids from exon 12 of RET (e.g., from the amino acid sequence of RET following the fusion junction with GOLGA5, e.g., of the RET sequence shown in SEQ ID NO:6).

In one embodiment, the GOLGA5-RET fusion polypeptide includes a tyrosine kinase domain or a functional fragment thereof. In a related aspect, the invention features GOLGA5-RET fusion polypeptide or fragments operatively linked to heterologous polypeptides to form fusion proteins.

In another embodiment, the GOLGA5-RET fusion polypeptide or fragment is a peptide, e.g., an immunogenic peptide or protein. Such a protein or peptide contains a fusion junction described herein. Such immunogenic peptides or proteins can be used to raise antibodies specific to the fusion protein. In other embodiments, such immunogenic peptides or proteins can be used for vaccine preparation. The vaccine preparation can include other components, e.g., an adjuvant.

In another aspect, the invention features antibody molecules that bind to a GOLGA5-RET fusion polypeptide or fragment described herein. In embodiments, the antibody can distinguish wild type RET (or GOLGA5) from GOLGA5-RET.

### Detection Reagents and Detection of Mutations

In another aspect, the invention features a detection reagent, e.g., a purified or an isolated preparation thereof. Detection reagents can distinguish a nucleic acid, or protein sequence, having a breakpoint, e.g., a GOLGA5-RET breakpoint; from a reference sequence. In one embodiment, the detection reagent detects (e.g., specifically detects) a GOLGA5-RET fusion nucleic acid or a polypeptide, e.g., distinguishes a wild type RET or another RET fusion (or GOLGA5) from a GOLGA5-RET nucleic acid (e.g., as described herein in SEQ ID NO:47 and SEQ ID NO:5); or a GOLGA5-RET polypeptide (e.g., as described herein in SEQ ID NO:48 and SEQ ID NO:6).

Detection reagents, e.g., nucleic acid-based detection reagents, can be used to identify mutations in a target nucleic acid, e.g., DNA, e.g., genomic DNA or cDNA, or RNA, e.g., in a sample, e.g., a sample of nucleic acid derived from a neoplastic or tumor cell, e.g., a melanocytic neoplasm, melanoma or metastatic cell. Detection reagents, e.g., antibody-based detection reagents, can be used to identify mutations in a target protein, e.g., in a sample, e.g., a sample of protein derived from, or produced by, a neoplastic or tumor cell, e.g., a melanocytic neoplasm, melanoma or metastatic cell.

### Method of Treatment

In a related aspect, a method of inhibiting, reducing, or treating a hyperproliferative disorder, e.g., a neoplasm (including benign, pre-malignant or malignant (e.g., a cancer), in a subject is provided. The method includes administering to the subject a preselected therapeutic agent, e.g., an anti-cancer agent (e.g., a kinase inhibitor), as a single agent, or in combination, in an amount sufficient to reduce, inhibit or treat the activity or expression of GOLGA5-RET (e.g., a GOLGA5-RET fusion described herein), thereby inhibiting, reducing, or treating the hyperproliferative disorder in the subject.

In one embodiment, the subject treated has a GOLGA5-RET fusion; e.g., the subject has a tumor or cancer harboring a GOLGA5-RET fusion. In other embodiments, the subject has been previously identified as having a GOLGA5-RET fusion. In yet other embodiments, the subject has been previously identified as being likely or unlikely to respond to treatment with a protein kinase inhibitor, e.g., a subject that has previously participated in a clinical trial. In other embodiments, the subject has been previously identified as being likely or unlikely to respond to treatment with a protein kinase inhibitor, based on the presence of the GOLGA5-RET fusion. In one embodiment, the subject is a mammal, e.g., a human. In one embodiment, the subject has, or at risk of having a cancer at any stage of disease. In other embodiments, the subject is a patient, e.g., a cancer patient.

In certain embodiments, the neoplasm or neoplastic cell is a benign, pre-malignant, malignant (cancer) or metastasis. In certain embodiments, the cancer is a solid tumor, a soft tissue tumor, or a metastatic lesion.

In one embodiment, the neoplasm or neoplastic cell is a benign, pre-malignant, malignant (cancer) or metastasis. In certain embodiments, the cancer is a solid tumor, a soft tissue tumor, or a metastatic lesion. In one embodiment, the cancer is a carcinoma. In one embodiment, the cancer is an adenocarcinoma. In an embodiment, the cancer is a lung adenocarcinoma. In one embodiment, the cancer is chosen from a lung cancer, a pancreatic cancer, melanoma, a colorectal cancer, an esophageal-gastric cancer, a thyroid cancer, or an adenocarcinoma. In one embodiment, the cancer is thyroid cancer. In one embodiment, the cancer is a papillary thyroid carcinoma. In one embodiment, the cancer is a lung adenocarcinoma. In other embodiment, the lung cancer is chosen from one or more of the following: non-small cell lung cancer (NSCLC), small cell lung cancer (SCLC), squamous cell carcinoma (SCC), adenocarcinoma of the lung, bronchogenic carcinoma, or a combination thereof. In one embodiment, the lung cancer is NSCLC or SCC.

In one embodiment, the anti-cancer agent is a RET inhibitor. In one embodiment, the anti-cancer agent is a GOLGA5 inhibitor. In one embodiment, the anti-cancer agent is a kinase inhibitor. In one embodiment, the anti-cancer agent is a RET inhibitor, e.g., CEP-701 and CEP-751; 2-indolinone, e.g., RPI-1; and quinazoline, e.g., ZD6474; or TG101209.

### KIF5B-RET Fusions

In one embodiment, a fusion includes an in-frame fusion of an exon of kinesin family member 5B (KIF5B), e.g., one more exons of KIF5B (e.g., one or more of exons 1-16 of KIF5B) or a fragment thereof, and an exon of ret proto-oncogene (RET), e.g., one or more exons of a RET (e.g., one or more of exons 12-19 of RET) or a fragment thereof. For example, the KIF5B-RET fusion can include an in-frame fusion within an intron of KIF5B (e.g., intron 16) or a fragment thereof, with an intron of RET (e.g., intron 11) or a fragment thereof. In one embodiment, the fusion of the KIF5B-RET fusion comprises the nucleotide sequence of: chromosome 10 at one or more of a nucleotide (plus or minus 10, 20, 30, 50, 60, 70, 80, 100 or more nucleotides) and chromosome 10 at one or more of a nucleotide (plus or minus 10, 20, 30, 50, 60, 70, 80, 100 or more nucleotides). In one embodiment, the KIF5B-RET fusion is a translocation, e.g., a translocation of a portion of chromosome 10 and a portion of chromosome 10.

In certain embodiments, the KIF5B-RET fusion is in a 5′-KIF5B to 3′-RET configuration (also referred to herein as “5′-KIF5B-RET-3′).” The term “fusion” or “fusion molecule” can refer to a polypeptide or a nucleic acid fusion, depending on the context. It may include a full-length sequence of a fusion or a fragment thereof, e.g., a fusion junction (e.g., a fragment including a portion of KIF5B and a portion of RET, e.g., a portion of the KIF5B-RET fusion described herein). In one embodiment, the KIF5B-RET fusion polypeptide includes a fragment of the amino acid sequence shown in SEQ ID NO:56 and a fragment of the amino acid sequence shown in SEQ ID NO:6, or an amino acid sequence substantially identical thereto. In another embodiment, the KIF5B-RET fusion nucleic acid includes a fragment of the nucleotide sequence shown in SEQ ID NO:55 and a fragment of the nucleotide sequence shown in SEQ ID NO:5, or a nucleotide sequence substantially identical thereto. In one embodiment, the KIF5B-RET fusion polypeptide comprises sufficient KIF5B and sufficient RET sequence such that the 5′ KIF5B-3′ RET fusion has kinase activity. e.g., has elevated activity. In any event, the fusion causes activation in the cells harboring it of oncogenic signaling pathways.

In certain embodiments, the KIF5B-RET fusion comprises one or more (or all of) exons 1-16 from KIF5B and one or more (or all of) exons 12-19 of RET (e.g., one or more of the exons shown in SEQ ID NO:55 and SEQ ID NO:5. In another embodiment, the KIF5B-RET fusion comprises one or more (or all of) exons 1-16 of KIF5B and one or more (or all of) exons 12-19 of RET. In certain embodiments, the KIF5B-RET fusion comprises at least 1, 2, 3, 4, 5, 6, 7, 8, 9, 10, 11, 12, 13, 14, 15, 16 or more exons (or encoded exons) from KIF5B and at least 1, 2, 3, 4, 5, 6, 7, 8 or more exons (or encoded exons) from RET (e.g., from the KIF5B and RET sequences shown in SEQ ID NO:55 and SEQ ID NO:56 and SEQ ID NO:5 and SEQ ID NO:6.

In certain embodiments, the KIF5B-RET fusion comprises exons 1-16 or a fragment thereof from KIF5B, and exons 12-19 or a fragment thereof from RET (e.g., as shown in SEQ ID NO:55 and SEQ ID NO:5). In one embodiment, the KIF5B-RET fusion comprises at least 5, 10, 15, 20, 30, 40, 50 or more amino acids from exons 1-16 of KIF5B (e.g., from the amino acid sequence of KIF5B as shown in SEQ ID NO:56 (e.g., from the amino acid sequence of KIF5B preceding the fusion junction with RET, and at least 5, 10, 15, 20, 30, 40, 50 or more amino acids from exons 12-19 of RET (e.g., from the amino acid sequence of RET as shown in SEQ ID NO:6). In another embodiment, the KIF5B-RET fusion comprises at least 6, 12, 15, 20, 25, 50, 75, 100 or more nucleotides from exons 1-16 of KIF5B (e.g., from the nucleotide sequence of KIF5B as shown in SEQ ID NO: 55 (e.g., from the nucleotide sequence of KIF5B preceding the fusion junction with RET); and at least 6, 12, 15, 20, 25, 50, 75, 100 or more nucleotides from exons 12-19 of RET (e.g., from the nucleotide sequence of RET as shown in SEQ ID NO:5).

### KIF5B-RET Nucleic Acid Molecules

In one aspect, the invention features a nucleic acid molecule (e.g., an isolated or purified) nucleic acid molecule that includes a fragment of a KIF5B gene and a fragment of a RET gene. In one embodiment, the nucleotide sequence encodes a KIF5B-RET fusion polypeptide that includes a tyrosine kinase domain or a functional fragment thereof. In another embodiment, the nucleotide sequence encodes a fragment of the RET polypeptide including the amino acid sequence of SEQ ID NO:6 or a fragment thereof, or a sequence substantially identical thereto. In other embodiments, the nucleic acid molecule includes a fragment of the KIF5B gene encoding the amino acid sequence of SEQ ID NO:56 or a fragment thereof, or a sequence substantially identical thereto. In yet other embodiments, the nucleic acid molecule includes a nucleotide sequence encoding the amino acid sequence shown in SEQ ID NO:56, or a fragment thereof, and the amino acid sequence shown in SEQ ID NO:6 or a fragment thereof, or a sequence substantially identical thereto.

In one embodiment, the nucleic acid molecule includes a fusion, e.g., an in-frame fusion, between an intron of KIF5B (e.g., intron 16, or a fragment thereof), and an intron of RET (e.g., intron 11, or a fragment thereof). The KIF5B-RET fusion can comprise a fusion of the nucleotide sequence of: chromosome 10 at one or more of a nucleotide (plus or minus 10, 20, 30, 50, 60, 70, 80, 100 nucleotides) and chromosome 10 at one or more of a nucleotide (plus or minus 10, 20, 30, 50, 60, 70, 80, 100 nucleotides), or a fragment thereof. In one embodiment, the KIF5B-RET fusion comprises a fusion of the nucleotide sequence of: chromosome 10 at one or more of a nucleotide (plus or minus 10, 20, 30, 50, 60, 70, 80, 100 nucleotides) and chromosome 10 at one or more of a nucleotide (plus or minus 10, 20, 30, 50, 60, 70, 80, 100 nucleotides), or a fragment thereof.

In another embodiment, the KIF5B-RET fusion comprises a nucleotide sequence (e.g., a fragment of a nucleotide sequence) shown in SEQ ID NO:55 and a nucleotide sequence (e.g., a fragment of a nucleotide sequence) shown in SEQ ID NO:5, or a fragment of the fusion. In one embodiment, the KIF5B-RET fusion comprises a nucleotide sequence substantially identical to the nucleotide sequence (e.g., a fragment of a nucleotide sequence) shown in SEQ ID NO:55 and the nucleotide sequence (e.g., a fragment of a nucleotide sequence) shown SEQ ID NO:5, or a fragment of the fusion. In one embodiment, the KIF5B-RET fusion comprises a nucleotide sequence at least 70%, at least 75%, at least 80%, at least 85%, at least 90%, at least 95%, at least 99%, at least 99.5 or greater, identical to the nucleotide sequence (e.g., a fragment of a nucleotide sequence) shown in SEQ ID NO:55 and to the nucleotide sequence (e.g., a fragment of a nucleotide sequence) shown in SEQ ID NO:5. In one embodiment, the KIF5B-RET fusion comprises a nucleotide sequence containing at least 25, 50, 100, 150, 200, 500, 1000, 1500, 2000, 2500, 3000, or more nucleotides of the nucleotide sequence shown in SEQ ID NO:55 and a nucleotide sequence containing at least 25, 50, 100, 150, 200, 500, 1000, 1500, 2000, 2500, 3000, or more nucleotides of the nucleotide sequence shown in SEQ ID NO:5. In one embodiment, the KIF5B-RET fusion comprises a nucleotide sequence containing at least 25, 50, 100, 150, 200, 500, 1000, 1500, 2000, 2500, 3000, or more contiguous nucleotides of the nucleotide sequence shown in SEQ ID NO:55 and a nucleotide sequence containing at least 25, 50, 100, 150, 200, 500, 1000, 1500, 2000, 2500, 3000, or more nucleotides of the nucleotide sequence shown in SEQ ID NO:5.

In another embodiment, the nucleic acid molecule includes a fusion, e.g., an in-frame fusion, of at least 1, 2, 3, 4, 5, 6, 7, 8, 9, 10, 11, 12, 13, 14, 15, 16 or more exons of KIF5B or a fragment thereof (e.g., one or more of exons 1-16 of KIF5B or a fragment thereof), and at least 1, 2, 3, 4, 5, 6, 7, 8 or more exons of RET or a fragment thereof (e.g., one or more of exons 12-19 of RET or a fragment thereof). In yet other embodiments, the nucleic acid molecule includes a fragment the nucleotide sequence shown in SEQ ID NO:55 and a fragment of the nucleotide sequence shown in SEQ ID NO:5 or a fragment of the fusion, or a sequence substantially identical thereto.

In one embodiment, the nucleic acid molecule is complementary to at least a portion of a nucleotide sequence disclosed herein, e.g., is capable of hybridizing under a stringency condition described herein to SEQ ID NO:55 and/or SEQ ID NO:5, or a fragment thereof. In yet another embodiment, the nucleic acid molecule hybridizes to a nucleotide sequence that is complementary to at least a portion of a nucleotide sequence disclosed herein, e.g., is capable of hybridizing under a stringency condition to a nucleotide sequence complementary to SEQ ID NO:55 and/or SEQ ID NO:5, or a fragment thereof. The nucleotide sequence of a cDNA encoding an exemplary 5′ KIF5B-3′ RET fusion is shown in at least exon 16 (e.g., exons 1-16) of SEQ ID NO:55 and at least exon 12 (e.g., exons 12-19) of SEQ ID NO:5, and the predicted amino acid sequence is shown in the corresponding encoded exons of SEQ ID NO:56 and the corresponding encoded exons of SEQ ID NO:6, respectively.

In an embodiment the KIF5B-RET nucleic acid molecule comprises sufficient KIF5B and sufficient RET sequence such that the encoded 5′ KIF5B-3′ RET fusion has kinase activity, e.g., has elevated activity. In certain embodiments, the 5′ KIF5B-3′ RET fusion comprises exons 1-16 from KIF5B and exons 12-19 from RET. In certain embodiments, the KIF5B-RET fusion comprises at least 1, 2, 3, 4, 5, 6, 7, 8, 9, 10, 11, 12, 13, 14, 15, 16 or more exons from KIF5B and at least at least 1, 2, 3, 4, 5, 6, 7, 8 or more exons of RET. In certain embodiments, the KIF5B-RET fusion comprises a fusion of exon 16 from KIF5B and exon 12 from RET. In another embodiment, the KIF5B-RET fusion comprises 1, 2, 3, 4, 5, 6, 7, 8, 9, 10, 11, 12, 13, 14, 15, 16 or more exons of KIF5B; and at least 1, 2, 3, 4, 5, 6, 7, 8 or more exons of RET.

In one embodiment, the nucleic acid molecule includes a nucleotide sequence that has an in-frame fusion of intron 16 of KIF5B (e.g., NM_{—}004521.2) with intron 11 of RET (e.g., NM_{—}020630). In another embodiment, the nucleic acid molecule includes a nucleotide sequence that includes a breakpoint. For example, the nucleic acid molecule includes a nucleotide sequence that includes the fusion junction between the KIF5B gene and the RET gene, e.g., the breakpoint between intron 16 of KIF5B and intron 11 of RET. In other embodiments, the nucleic acid molecules includes a nucleotide sequence of one or more of nucleotide of chromosome 10 coupled to (e.g., directly or indirectly juxtaposed to) one or more of nucleotide of chromosome 10. In one embodiment, the nucleic acid molecule includes the nucleotide sequence of: chromosome 10 at one or more of a nucleotide plus or minus 10, 20, 30, 40 50, 60, 80, 100, 150 nucleotides and chromosome 10 at one or more of a nucleotide plus or minus 10, 20, 30, 40 50, 60, 80, 100, 150 nucleotides (corresponding to the breakpoint of a KIF5B-RET fusion), or a fragment thereof, or a sequence substantially identical thereto. In one embodiment, the nucleic acid molecule is complementary to at least a portion of a nucleotide sequence disclosed herein, e.g., is capable of hybridizing under a stringency condition described herein to SEQ ID NO:55 and/or SEQ ID NO:5 or a fragment thereof. In yet other embodiment, the nucleic acid molecule hybridizes to a nucleotide sequence that is complementary to at least a portion of a nucleotide sequence disclosed herein, e.g., is capable of hybridizing under a stringency condition described herein to a nucleotide sequence complementary to SEQ ID NO:55 or SEQ ID NO:5 or a fragment thereof.

In another embodiment, the KIF5B-RET fusion nucleic acid comprises at least 6, 12, 15, 20, 25, 50, 75, 100 or more nucleotides from exon 16 of KIF5B (e.g., from the nucleotide sequence of KIF5B preceding the fusion junction with RET, e.g., of the KIF5B sequence shown in SEQ ID NO:55), and at least 6, 12, 15, 20, 25, 50, 75, 100 or more nucleotides from exon 12 of RET (e.g., from the nucleotide sequence of RET following the fusion junction with KIF5B, e.g., of the RET sequence shown in SEQ ID NO:5).

In one embodiment, the KIF5B-RET fusion includes the full sequence or a fragment of the nucleotide sequence shown in SEQ ID NO:53 and SEQ ID NO:100, or a nucleotide sequence substantially identical thereto. In another embodiment, the KIF5B-RET fusion polypeptide includes the full sequence or a fragment of the amino acid sequence shown in SEQ ID NO:54 and SEQ ID NO:101, or an amino acid sequence substantially identical thereto, for example at least 70% or at least 80% or at least 90% identical or even more.

In other embodiments, the nucleic acid molecule includes a nucleotide sequence encoding a KIF5B-RET fusion polypeptide that includes a fragment of a KIF5B gene and a fragment of a RET gene. In one embodiment, the nucleotide sequence encodes a KIF5B-RET fusion polypeptide that includes e.g., a tyrosine kinase domain or a functional fragment thereof. In yet other embodiments, the nucleic acid molecule includes a nucleotide sequence encoding the amino acid sequence (e.g., a fragment of the amino acid sequence) shown in SEQ ID NO:56 and a nucleotide sequence encoding the amino acid sequence (e.g., a fragment of the amino acid sequence) shown in SEQ ID NO:6, or a fragment of the fusion, or a sequence substantially identical thereto. In one embodiment, the encoded KIF5B-RET fusion polypeptide includes a tyrosine kinase domain or a functional fragment thereof.

In a related aspect, the invention features nucleic acid constructs that include the KIF5B-RET nucleic acid molecules described herein. In certain embodiments, the nucleic acid molecules are operatively linked to a native or a heterologous regulatory sequence. Also included are vectors and host cells that include the KIF5B-RET nucleic acid molecules described herein, e.g., vectors and host cells suitable for producing the nucleic acid molecules and polypeptides described herein.

In a related aspect, methods of producing the nucleic acid molecules and polypeptides described herein are also described.

In another aspect, the invention features nucleic acid molecules that reduce or inhibit the expression of a nucleic acid molecule that encodes a KIF5B-RET fusion described herein. Examples of such nucleic acid molecules include, for example, antisense molecules, ribozymes, RNAi, triple helix molecules that hybridize to a nucleic acid encoding KIF5B-RET, or a transcription regulatory region of KIF5B-RET, and blocks or reduces mRNA expression of KIF5B-RET.

### Nucleic Acid Detection and Capturing Reagents

The invention also features a nucleic acid molecule, e.g., nucleic acid fragment, suitable as probe, primer, bait or library member that includes, flanks, hybridizes to, which are useful for identifying, or are otherwise based on, the KIF5B-RET fusions described herein. In certain embodiments, the probe, primer or bait molecule is an oligonucleotide that allows capture, detection or isolation of a KIF5B-RET fusion nucleic acid molecule described herein. The oligonucleotide can comprise a nucleotide sequence substantially complementary to a fragment of the KIF5B-RET fusion nucleic acid molecules described herein. The sequence identity between the nucleic acid fragment, e.g., the oligonucleotide, and the target KIF5B-RET sequence need not be exact, so long as the sequences are sufficiently complementary to allow the capture, detection or isolation of the target sequence. In one embodiment, the nucleic acid fragment is a probe or primer that includes an oligonucleotide between about 5 and 25, e.g., between 10 and 20, or 10 and 15 nucleotides in length. In other embodiments, the nucleic acid fragment is a bait that includes an oligonucleotide between about 100 to 300 nucleotides, 130 and 230 nucleotides, or 150 and 200 nucleotides, in length.

In one embodiment, the nucleic acid fragment can be used to identify or capture, e.g., by hybridization, a KIF5B-RET fusion. For example, the nucleic acid fragment can be a probe, a primer, or a bait, for use in identifying or capturing, e.g., by hybridization, a KIF5B-RET fusion described herein. In one embodiment, the nucleic acid fragment can be useful for identifying or capturing a KIF5B-RET breakpoint, e.g., the nucleotide sequence of: chromosome 10 at nucleotide plus or minus 10, 20, 30, 40, 50, 60, 80, 100, 150 nucleotides and chromosome 10 at nucleotide plus or minus 10, 20, 30, 40 50, 60, 80, 100, 150 nucleotides.

In one embodiment, the nucleic acid fragment hybridizes to a nucleotide sequence within a chromosomal rearrangement that creates an in-frame fusion of intron 16 of KIF5B with intron 11 of RET. In one embodiment, the nucleic acid fragment hybridizes to a nucleotide sequence in the region In other embodiments, the nucleic acid molecules includes a nucleotide sequence in the region of a nucleotide sequence of chromosome 10 coupled to (e.g., juxtaposed to) nucleotides in the region of a nucleotide sequence Y of chromosome 10. In one embodiment, the nucleic acid fragment hybridizes to a nucleotide sequence that includes a breakpoint, e.g., the nucleotide sequence of: chromosome 10 at nucleotide plus or minus 10, 20, 30, 40 50, 60, 80, 100, 150 or more nucleotides and chromosome 10 at nucleotide plus or minus 10, 20, 30, 40 50, 60, 80, 100, 150 or more nucleotides. For example, the nucleic acid fragment can hybridize to a nucleotide sequence that includes the fusion junction between the KIF5B gene and the RET gene, e.g., a nucleotide sequence that includes a portion of a nucleotide sequence within intron 16 of a KIF5B gene and intron 11 of a RET gene.

In another embodiment, the nucleic acid fragment hybridizes to a nucleotide sequence that comprises at least 6, 12, 15, 20, 25, 50, 75, 100, 150 or more nucleotides from exon 16 of KIF5B (e.g., from the nucleotide sequence of KIF5B preceding the fusion junction with RET, e.g., of the KIF5B sequence shown in SEQ ID NO:55), and at least 6, 12, 15, 20, 25, 50, 75, 100, 150 or more nucleotides from exon 12 of RET (e.g., from the nucleotide sequence of RET following the fusion junction with KIF5B, e.g., of the RET sequence shown in SEQ ID NO:5).

The probes or primers described herein can be used, for example, for FISH detection or PCR amplification. In one exemplary embodiment where detection is based on PCR, amplification of the KIF5B-RET fusion junction fusion junction can be performed using a primer or a primer pair, e.g., for amplifying a sequence flanking the fusion junctions described herein, e.g., the mutations or the junction of a chromosomal rearrangement described herein, e.g., KIF5B-RET.

In one embodiment, a pair of isolated oligonucleotide primers can amplify a region containing or adjacent to a position in the KIF5B-RET fusion. For example, forward primers can be designed to hybridize to a nucleotide sequence within KIF5B genomic or mRNA sequence (e.g., a nucleotide sequence within exon 16 of KIF5B of SEQ ID NO:55, and the reverse primers can be designed to hybridize to a nucleotide sequence of RET (e.g., a nucleotide sequence within exon 12 of RET, of SEQ ID NO:5.

In another embodiment, the nucleic acid fragments can be used to identify, e.g., by hybridization, a KIF5B-RET fusion. In one embodiment, the nucleic acid fragment hybridizes to a nucleotide sequence that includes a fusion junction between the KIF5B transcript and the RET transcript.

In other embodiments, the nucleic acid fragment includes a bait that comprises a nucleotide sequence that hybridizes to a KIF5B-RET fusion nucleic acid molecule described herein, and thereby allows the capture or isolation said nucleic acid molecule. In one embodiment, a bait is suitable for solution phase hybridization. In other embodiments, a bait includes a binding entity, e.g., an affinity tag, that allows capture and separation, e.g., by binding to a binding entity, of a hybrid formed by a bait and a nucleic acid hybridized to the bait.

In other embodiments, the nucleic acid fragment includes a library member comprising a KIF5B-RET nucleic acid molecule described herein. In one embodiment, the library member includes a rearrangement that results in a KIF5B-RET fusion described herein.

The nucleic acid fragment can be detectably labeled with, e.g., a radiolabel, a fluorescent label, a bioluminescent label, a chemiluminescent label, an enzyme label, a binding pair label, or can include an affinity tag; a tag, or identifier (e.g., an adaptor, barcode or other sequence identifier).

### KIF5B-RET Fusion Polypeptides

In another embodiment, the KIF5B-RET fusion comprises an amino acid sequence (e.g., a fragment of the amino acid sequence) shown in SEQ ID NO:56 and an amino acid sequence (e.g., a fragment of the amino acid sequence) shown in SEQ ID NO:6, or a fragment of the fusion. In one embodiment, the KIF5B-RET fusion comprises an amino acid sequence substantially identical to the amino acid sequence (e.g., a fragment of the amino acid sequence) shown in SEQ ID NO:56 and the amino acid sequence (e.g., a fragment of the amino acid sequence) shown in SEQ ID NO:6, or a fragment thereof. In one embodiment, the KIF5B-RET fusion comprises an amino acid sequence at least 70%, at least 75%, at least 80%, at least 85%, at least 90%, at least 95%, at least 99%, at least 99.5 or greater, identical to the amino acid sequence (e.g., a fragment of the amino acid sequence) shown in SEQ ID NO:56 and the amino acid sequence (e.g., a fragment of the amino acid sequence) shown in SEQ ID NO:6. In one embodiment, the KIF5B-RET fusion comprises a sequence containing at least 10, 20, 50, 100, 500, 600, 700, 800, 900, 1000, or more amino acids of the amino acid sequence shown in SEQ ID NO:56 and SEQ ID NO:6. In one embodiment, the KIF5B-RET fusion comprises an amino acid sequence containing at least 5, 10, 20, 50, 100, 500, 600, 700, 800, 900, 1000, or more contiguous amino acids of the amino acid sequence shown in SEQ ID NO:56 and at least 5, 10, 20, 50, 100, 500, 600, 700, 800, 900, 1000, or more contiguous amino acids of the amino acid sequence shown in SEQ ID NO:6. In one embodiment, the 5′ KIF5B-3′ RET fusion polypeptide includes a receptor tyrosine kinase domain or a functional fragment thereof. In an embodiment, the 5′KIF5B-3′RET fusion polypeptide comprises sufficient RET and sufficient KIF5B sequence such that it has kinase activity, e.g., has elevated activity or in any event promotes activation of oncogenic signaling pathways.

In another aspect, the disclosure features a KIF5B-RET fusion polypeptide (e.g., a purified KIF5B-RET fusion polypeptide), a biologically active or antigenic fragment thereof, as well as reagents (e.g., antibody molecules that bind to a KIF5B-RET fusion polypeptide), methods for modulating a KIF5B-RET polypeptide activity and detection of a KIF5B-RET polypeptide.

In one embodiment, the KIF5B-RET fusion polypeptide has at least one biological activity. In one embodiment, at least one biological activity of the KIF5B-RET fusion polypeptide is reduced or inhibited by an anti-cancer drug, e.g., a KIF5B inhibitor, a RET inhibitor. In one embodiment, at least one biological activity of the KIF5B-RET fusion polypeptide is reduced or inhibited by a RET inhibitor. In one embodiment, at least one biological activity of the KIF5B-RET fusion polypeptide is reduced or inhibited by a KIF5B inhibitor. In one embodiment, at least one biological activity of the KIF5B-RET fusion polypeptide is reduced or inhibited by a RET inhibitor, e.g., CEP-701 and CEP-751; 2-indolinone, e.g., RPI-1; and quinazoline, e.g., ZD6474; or TG101209.

In yet other embodiments, the KIF5B-RET fusion polypeptide is encoded by a nucleic acid molecule described herein. In one embodiment, the KIF5B-RET fusion polypeptide is encoded by an in-frame fusion of intron 16 of KIF5B with intron 11 of RET (e.g., a sequence on chromosome 10 and a sequence on chromosome 10). In another embodiment, the KIF5B-RET fusion polypeptide includes an amino acid sequence encoded by a nucleotide sequence comprising a fusion junction between the KIF5B transcript and the RET transcript.

In certain embodiments, the KIF5B-RET fusion polypeptide comprises one or more of encoded exons 1-16 from KIF5B and one or more of encoded exons 12-19 of RET. In certain embodiments, the KIF5B-RET fusion polypeptide comprises at least 1, 2, 3, 4, 5, 6, 7, 8, 9, 10, 11, 12, 13, 14, 15, 16 or more encoded exons of KIF5B and at least at least 1, 2, 3, 4, 5, 6, 7, 8 or more encoded exons of RET. In certain embodiments, the KIF5B-RET fusion polypeptide comprises a fusion of encoded exon 16 from KIF5B and encoded exon 12 from RET (or a fragment thereof). In other embodiments, the fusion comprises at least 1, 2, 3, 4, 5, 6, 7, 8, 9, 10, 11, 12, 13, 14, 15, 16 or more encoded exons of KIF5B; and at least 1, 2, 3, 4, 5, 6, 7, 8 or more encoded exons of RET. In certain embodiments, the KIF5B-RET fusion polypeptide comprises encoded exons 1-16 from KIF5B and exons 12-19 of RET. In certain embodiments, the 5′ KIF5B-3′ RET fusion polypeptide comprises a fusion junction of the sequence of exon 16 from KIF5B and the sequence of exon 12 from RET.

In certain embodiments, the KIF5B-RET fusion comprises the amino acid sequence corresponding to exon 16 or a fragment thereof from KIF5B, and the amino acid sequence corresponding to exon 12 or a fragment thereof from RET (e.g., as shown in SEQ ID NO:56 and SEQ ID NO:6). In one embodiment, the KIF5B-RET fusion comprises at least 5, 10, 15, 20 or more amino acids from exon 16 of KIF5B (e.g., from the amino acid sequence of KIF5B preceding the fusion junction with RET, e.g., of the KIF5B sequence shown in SEQ ID NO:56), and at least 5, 10, 15, 20 or more amino acids from exon 12 of RET (e.g., from the amino acid sequence of RET following the fusion junction with KIF5B, e.g., of the RET sequence shown in SEQ ID NO:6).

In one embodiment, the KIF5B-RET fusion polypeptide includes a tyrosine kinase domain or a functional fragment thereof. In a related aspect, the invention features KIF5B-RET fusion polypeptide or fragments operatively linked to heterologous polypeptides to form fusion proteins.

In another embodiment, the KIF5B-RET fusion polypeptide or fragment is a peptide, e.g., an immunogenic peptide or protein containing a fusion junction described herein. Such immunogenic peptides or proteins can be used to raise antibodies specific to the fusion protein. In other embodiments, such immunogenic peptides or proteins can be used for vaccine preparation. The vaccine preparation can include other components, e.g., an adjuvant.

In another aspect, the invention features antibody molecules that bind to a KIF5B-RET fusion polypeptide or fragment described herein. In embodiments, the antibody can distinguish wild type RET (or KIF5B) from KIF5B-RET.

### Detection Reagents and Detection of Mutations

In another aspect, the invention features a detection reagent, e.g., a purified or an isolated preparation thereof. Detection reagents can distinguish a nucleic acid, or protein sequence, having a breakpoint, e.g., a KIF5B-RET breakpoint; from a reference sequence. In one embodiment, the detection reagent detects (e.g., specifically detects) a KIF5B-RET fusion nucleic acid or a polypeptide, e.g., distinguishes a wild type RET or another RET fusion (or KIF5B) from a KIF5B-RET nucleic acid (e.g., as described herein in SEQ ID NO:55 and SEQ ID NO:5); or a KIF5B-RET polypeptide (e.g., as described herein in SEQ ID NO:56 and SEQ ID NO:6).

Detection reagents, e.g., nucleic acid-based detection reagents, can be used to identify mutations in a target nucleic acid, e.g., DNA, e.g., genomic DNA or cDNA, or RNA. e.g., in a sample, e.g., a sample of nucleic acid derived from a neoplastic or tumor cell, e.g., a melanocytic neoplasm, melanoma or metastatic cell. Detection reagents, e.g., antibody-based detection reagents, can be used to identify mutations in a target protein, e.g., in a sample, e.g., a sample of protein derived from, or produced by, a neoplastic or tumor cell, e.g., a melanocytic neoplasm, melanoma or metastatic cell.

### Method of Treatment

In a related aspect, a method of inhibiting, reducing, or treating a hyperproliferative disorder, e.g., a neoplasm (including benign, pre-malignant or malignant (e.g., a cancer), in a subject is provided. The method includes administering to the subject a preselected therapeutic agent, e.g., an anti-cancer agent (e.g., a kinase inhibitor), as a single agent, or in combination, in an amount sufficient to reduce, inhibit or treat the activity or expression of KIF5B-RET (e.g., a KIF5B-RET fusion described herein), thereby inhibiting, reducing, or treating the hyperproliferative disorder in the subject.

In one embodiment, the subject treated has a KIF5B-RET fusion; e.g., the subject has a tumor or cancer harboring a KIF5B-RET fusion. In other embodiments, the subject has been previously identified as having a KIF5B-RET fusion. In yet other embodiments, the subject has been previously identified as being likely or unlikely to respond to treatment with a protein kinase inhibitor, e.g., a subject that has previously participated in a clinical trial. In other embodiments, the subject has been previously identified as being likely or unlikely to respond to treatment with a protein kinase inhibitor, based on the presence of the KIF5B-RET fusion. In one embodiment, the subject is a mammal, e.g., a human. In one embodiment, the subject has, or at risk of having a cancer at any stage of disease. In other embodiments, the subject is a patient, e.g., a cancer patient.

In certain embodiments, the neoplasm or neoplastic cell is a benign, pre-malignant, malignant (cancer) or metastasis. In certain embodiments, the cancer is a solid tumor, a soft tissue tumor, or a metastatic lesion.

In one embodiment, the neoplasm or neoplastic cell is a benign, pre-malignant, malignant (cancer) or metastasis. In certain embodiments, the cancer is a solid tumor, a soft tissue tumor, or a metastatic lesion. In one embodiment, the cancer is a carcinoma. In one embodiment, the cancer is an adenocarcinoma. In an embodiment, the cancer is a lung adenocarcinoma. In one embodiment, the cancer is chosen from a lung cancer, a pancreatic cancer, melanoma, a colorectal cancer, an esophageal-gastric cancer, a thyroid cancer, or an adenocarcinoma. In one embodiment, the cancer is thyroid cancer. In one embodiment, the cancer is a papillary thyroid carcinoma. In one embodiment, the cancer is a lung adenocarcinoma. In other embodiment, the lung cancer is chosen from one or more of the following: non-small cell lung cancer (NSCLC), small cell lung cancer (SCLC), squamous cell carcinoma (SCC), adenocarcinoma of the lung, bronchogenic carcinoma, or a combination thereof. In one embodiment, the lung cancer is NSCLC or SCC.

In one embodiment, the anti-cancer agent is a RET inhibitor. In one embodiment, the anti-cancer agent is a KIF5B inhibitor. In one embodiment, the anti-cancer agent is a kinase inhibitor. In one embodiment, the anti-cancer agent is a RET inhibitor, e.g., CEP-701 and CEP-751; 2-indolinone, e.g., RPI-1; and quinazoline, e.g., ZD6474; or TG101209.

### TP53-NTRK1 Fusions

In one embodiment, a fusion includes an in-frame fusion of an exon of tumor protein 53 (TP53), e.g., one more exons of TP53 (e.g., one or more of exons 1-8 or exons 1-9 or exons 1-11 or exons 1-12 of TP53) or a fragment thereof, and an exon of neurotrophic tyrosine kinase receptor type 1 (NTRK1), e.g., one or more exons of NTRK1 (e.g., one or more of exons 9-17 of NTRK1) or a fragment thereof. For example, the TP53-NTRK1 fusion can include an in-frame fusion within an intron of TP53 (e.g., intron 8 or intron 9 or intron 11 or intron 12) or a fragment thereof, with an intron of NTRK1 (e.g., intron 8) or a fragment thereof. In one embodiment, the fusion of the TP53-NTRK1 fusion comprises the nucleotide sequence of: chromosome 17 at one or more of a nucleotide (plus or minus 10, 20, 30, 50, 60, 70, 80, 100 or more nucleotides) and chromosome 1 at one or more of a nucleotide (plus or minus 10, 20, 30, 50, 60, 70, 80, 100 or more nucleotides). In one embodiment, the TP53-NTRK1 fusion is a translocation. e.g., a translocation of a portion of chromosome 17 and a portion of chromosome 1.

In certain embodiments, the TP53-NTRK1 fusion is in a 5′-TP53 to 3′-NTRK1 configuration (also referred to herein as “5′-TP53-NTRK1-3′).” The term “fusion” or “fusion molecule” can refer to a polypeptide or a nucleic acid fusion, depending on the context. It may include a full-length sequence of a fusion or a fragment thereof, e.g., a fusion junction (e.g., a fragment including a portion of TP53 and a portion of NTRK1, e.g., a portion of the TP53-NTRK1 fusion described herein). In one embodiment, the TP53-NTRK1 fusion polypeptide includes a fragment of the amino acid sequence shown in SEQ ID NO:63 and a fragment of the amino acid sequence shown in SEQ ID NO:4, or an amino acid sequence substantially identical thereto. In another embodiment, the TP53-NTRK1 fusion nucleic acid includes a fragment of the nucleotide sequence shown in SEQ ID NO:62 and a fragment of the nucleotide sequence shown in SEQ ID NO:3, or a nucleotide sequence substantially identical thereto. In one embodiment, the TP53-NTRK1 fusion polypeptide comprises sufficient TP53 and sufficient NTRK1 sequence such that the 5′ TP53-3′ NTRK1 fusion has kinase activity, e.g., has elevated activity, e.g., tyrosine kinase activity, as compared with either wild type polypeptide. In any event, the fusion causes activation in the cells harboring it of oncogenic signaling pathways.

In certain embodiments, the TP53-NTRK1 fusion comprises one or more (or all of) exons 1-8 or exons 1-9 or exons 1-11 or exons 1-12 from TP53 and one or more (or all of) exons 9-17 of NTRK1 (e.g., one or more of the exons shown in SEQ ID NO:62 and SEQ ID NO:3. In another embodiment, the TP53-NTRK1 fusion comprises one or more (or all of) exons 1-8 or exons 1-9 or exons 1-1 or exons 1-12 of TP53 and one or more (or all of) exons 9-17 of NTRK1. In certain embodiments, the TP53-NTRK1 fusion comprises at least 1, 2, 3, 4, 5, 6, 7, 8, 9, 11, 12 or more exons (or encoded exons) from TP53 and at least 1, 2, 3, 4, 5, 6, 7, 8, 9 or more exons (or encoded exons) from NTRK1 (e.g., from the TP53 and NTRK1 sequences shown in SEQ ID NO:62 and SEQ ID NO:63 and SEQ ID NO:3 and SEQ ID NO:4.

In certain embodiments, the TP53-NTRK1 fusion comprises exons 1-8 or exons 1-9 or exons 1-11 or exons 1-12 or a fragment thereof from TP53, and exons 9-17 or a fragment thereof from NTRK1 (e.g., as shown in SEQ ID NO:62 and SEQ ID NO:3). In one embodiment, the TP53-NTRK1 fusion comprises at least 5, 10, 15, 20, 30, 40, 50 or more amino acids from exons 1-8 or exons 1-9 or exons 1-11 or exons 1-12 of TP53 (e.g., from the amino acid sequence of TP53 as shown in SEQ ID NO:63 (e.g., from the amino acid sequence of TP53 preceding the fusion junction with NTRK1, and at least 5, 10, 15, 20, 30, 40, 50 or more amino acids from exons 9-17 of NTRK1 (e.g., from the amino acid sequence of NTRK1 as shown SEQ ID NO:4). In another embodiment, the TP53-NTRK1 fusion comprises at least 6, 12, 15, 20, 25, 50, 75, 100 or more nucleotides from exons 1-8 or exons 1-9 or exons 1-11 or exons 1-12 of TP53 (e.g., from the nucleotide sequence of TP53 as shown in SEQ ID NO:62 (e.g., from the nucleotide sequence of TP53 preceding the fusion junction with NTRK1); and at least 6, 12, 15, 20, 25, 50, 75, 100 or more nucleotides from exons 9-17 of NTRK1 (e.g., from the nucleotide sequence of NTRK1 as shown in SEQ ID NO:3).

### TP53-NTRK1 Nucleic Acid Molecules

In one aspect, the invention features a nucleic acid molecule (e.g., an isolated or purified) nucleic acid molecule that includes a fragment of a TP53 gene and a fragment of a NTRK1 gene. In one embodiment, the nucleotide sequence encodes a TP53-NTRK1 fusion polypeptide that includes a tyrosine kinase domain or a functional fragment thereof. In another embodiment, the nucleotide sequence encodes a fragment of the NTRK1 polypeptide including the amino acid sequence of SEQ ID NO:4 or a fragment thereof, or a sequence substantially identical thereto. In other embodiments, the nucleic acid molecule includes a fragment of the TP53 gene encoding the amino acid sequence of SEQ ID NO:63 or a fragment thereof, or a sequence substantially identical thereto. In yet other embodiments, the nucleic acid molecule includes a nucleotide sequence encoding the amino acid sequence shown in SEQ ID NO:63, or a fragment thereof, and the amino acid sequence shown in SEQ ID NO:4 or a fragment thereof, or a sequence substantially identical thereto.

In one embodiment, the nucleic acid molecule includes a fusion, e.g., an in-frame fusion, between an intron of TP53 (e.g., intron 8 or intron 9 or intron 11 or intron 12, or a fragment thereof), and an intron of NTRK1 (e.g., intron 8, or a fragment thereof). The TP53-NTRK1 fusion can comprise a fusion of the nucleotide sequence of: chromosome 17 at one or more of a nucleotide (plus or minus 10, 20, 30, 50, 60, 70, 80, 100 nucleotides) and chromosome 1 at one or more of a nucleotide (plus or minus 10, 20, 30, 50, 60, 70, 80, 100 nucleotides), or a fragment thereof. In one embodiment, the TP53-NTRK1 fusion comprises a fusion of the nucleotide sequence of: chromosome 17 at one or more of a nucleotide (plus or minus 10, 20, 30, 50, 60, 70, 80, 100 nucleotides) and chromosome 1 at one or more of a nucleotide (plus or minus 10, 20, 30, 50, 60, 70, 80, 100 nucleotides), or a fragment thereof.

In another embodiment, the TP53-NTRK1 fusion comprises a nucleotide sequence (e.g., a fragment of a nucleotide sequence) shown in SEQ ID NO:62 and a nucleotide sequence (e.g., a fragment of a nucleotide sequence) shown in SEQ ID NO:3, or a fragment of the fusion. In one embodiment, the TP53-NTRK1 fusion comprises a nucleotide sequence substantially identical to the nucleotide sequence (e.g., a fragment of a nucleotide sequence) shown in SEQ ID NO:62 and the nucleotide sequence (e.g., a fragment of a nucleotide sequence) shown SEQ ID NO:3, or a fragment of the fusion. In one embodiment, the TP53-NTRK1 fusion comprises a nucleotide sequence at least 70%, at least 75%, at least 80%, at least 85%, at least 90%, at least 95%, at least 99%, at least 99.5 or greater, identical to the nucleotide sequence (e.g., a fragment of a nucleotide sequence) shown in SEQ ID NO:62 and to the nucleotide sequence (e.g., a fragment of a nucleotide sequence) shown in SEQ ID NO:3. In one embodiment, the TP53-NTRK1 fusion comprises a nucleotide sequence containing at least 25, 50, 100, 150, 200, 500, 1000, 1500, 2000, 2500, 3000, or more nucleotides of the nucleotide sequence shown in SEQ ID NO:62 and a nucleotide sequence containing at least 25, 50, 100, 150, 200, 500, 1000, 1500, 2000, 2500, 3000, or more nucleotides of the nucleotide sequence shown in SEQ ID NO:3. In one embodiment, the TP53-NTRK1 fusion comprises a nucleotide sequence containing at least 25, 50, 100, 150, 200, 500, 1000, 1500, 2000, 2500, 3000, or more contiguous nucleotides of the nucleotide sequence shown in 155 SEQ ID NO:62 and a nucleotide sequence containing at least 25, 50, 100, 150, 200, 500, 1000, 1500, 2000, 2500, 3000, or more nucleotides of the nucleotide sequence shown in SEQ ID NO:3.

In another embodiment, the nucleic acid molecule includes a fusion, e.g., an in-frame fusion, of at least 1, 2, 3, 4, 5, 6, 7, 8, 9, 11, 12 or more exons of TP53 or a fragment thereof (e.g., one or more of exons 1-8 or exons 1-9 or exons 1-11 or exons 1-12 of TP53 or a fragment thereof), and at least 1, 2, 3, 4, 5, 6, 7, 8, 9 or more exons of NTRK1 or a fragment thereof (e.g., one or more of exons 9-17 of NTRK1 or a fragment thereof). In yet other embodiments, the nucleic acid molecule includes a fragment the nucleotide sequence shown in SEQ ID NO:62 and a fragment of the nucleotide sequence shown in SEQ ID NO:3 or a fragment of the fusion, or a sequence substantially identical thereto.

In one embodiment, the nucleic acid molecule is complementary to at least a portion of a nucleotide sequence disclosed herein, e.g., is capable of hybridizing under a stringency condition described herein to SEQ ID NO:62 and/or SEQ ID NO:3, or a fragment thereof. In yet another embodiment, the nucleic acid molecule hybridizes to a nucleotide sequence that is complementary to at least a portion of a nucleotide sequence disclosed herein, e.g., is capable of hybridizing under a stringency condition to a nucleotide sequence complementary to SEQ ID NO:62 and/or SEQ ID NO:3, or a fragment thereof. The nucleotide sequence of a cDNA encoding an exemplary 5′ TP53-3′ NTRK1 fusion is shown in at least exon 8 or exon 9 or exon 11 or exon 12 (e.g., exons 1-8 or exons 1-9 or exons 1-11 or exons 1-12) of SEQ ID NO:62 and at least exon 9 (e.g., exons 9-17) of SEQ ID NO:3, and the predicted amino acid sequence is shown in the corresponding encoded exons of SEQ ID NO:63 and the corresponding encoded exons of SEQ ID NO:4, respectively.

In an embodiment the TP53-NTRK1 nucleic acid molecule comprises sufficient TP53 and sufficient NTRK1 sequence such that the encoded 5′ TP53-3′ NTRK1 fusion has kinase activity, e.g., has elevated activity. In certain embodiments, the 5′ TP53-3′ NTRK1 fusion comprises exons 1-8 or exons 1-9 or exons 1-11 or exons 1-12 from TP53 and exons 9-17 from NTRK1. In certain embodiments, the TP53-NTRK1 fusion comprises at least 1, 2, 3, 4, 5, 6, 7, 8, 9, 11, 12 or more exons from TP53 and at least at least 1, 2, 3, 4, 5, 6, 7, 8, 9 or more exons of NTRK1. In certain embodiments, the TP53-NTRK1 fusion comprises a fusion of exon 8 or exon 9 or exon 11 or exon 12 from TP53 and exon 9 from NTRK1. In another embodiment, the TP53-NTRK1 fusion comprises 1, 2, 3, 4, 5, 6, 7, 8, 9, 11, 12 or more exons of TP53; and at least 1, 2, 3, 4, 5, 6, 7, 8, 9 or more exons of NTRK1.

In one embodiment, the nucleic acid molecule includes a nucleotide sequence that has an in-frame fusion of intron 8 or intron 9 or intron 11 or intron 12 of TP53 (e.g., NM_{—}001126113) with intron 8 of NTRK1 (e.g., NM_{—}002529). In another embodiment, the nucleic acid molecule includes a nucleotide sequence that includes a breakpoint. For example, the nucleic acid molecule includes a nucleotide sequence that includes the fusion junction between the TP53 gene and the NTRK1 gene, e.g., the breakpoint between intron 8 or intron 9 or intron 11 or intron 12 of TP53 and intron 8 of NTRK1. In other embodiments, the nucleic acid molecules includes a nucleotide sequence of one or more of nucleotide of chromosome 17 coupled to (e.g., directly or indirectly juxtaposed to) one or more of nucleotide of chromosome 1. In one embodiment, the nucleic acid molecule includes the nucleotide sequence of: chromosome 17 at one or more of a nucleotide plus or minus 10, 20, 30, 40 50, 60, 80, 100, 150 nucleotides and chromosome 1 at one or more of a nucleotide plus or minus 10, 20, 30, 40 50, 60, 80, 100, 150 nucleotides (corresponding to the breakpoint of a TP53-NTRK1 fusion), or a fragment thereof, or a sequence substantially identical thereto. In one embodiment, the nucleic acid molecule is complementary to at least a portion of a nucleotide sequence disclosed herein, e.g., is capable of hybridizing under a stringency condition described herein to SEQ ID NO:62 and/or SEQ ID NO:3 or a fragment thereof. In yet other embodiment, the nucleic acid molecule hybridizes to a nucleotide sequence that is complementary to at least a portion of a nucleotide sequence disclosed herein, e.g., is capable of hybridizing under a stringency condition described herein to a nucleotide sequence complementary to SEQ ID NO:62 or SEQ ID NO:3 or a fragment thereof.

In another embodiment, the TP53-NTRK1 fusion nucleic acid comprises at least 6, 12, 15, 20, 25, 50, 75, 100 or more nucleotides from exon 8 or exon 9 or exon 11 or exon 12 of TP53 (e.g., from the nucleotide sequence of TP53 preceding the fusion junction with NTRK1, e.g., of the TP53 sequence shown in SEQ ID NO:62), and at least 6, 12, 15, 20, 25, 50, 75, 100 or more nucleotides from exon 9 of NTRK1 (e.g., from the nucleotide sequence of NTRK1 following the fusion junction with TP53, e.g., of the NTRK1 sequence shown in SEQ ID NO:3).

In other embodiments, the nucleic acid molecule includes a nucleotide sequence encoding a TP53-NTRK1 fusion polypeptide that includes a fragment of a TP53 gene and a fragment of a NTRK1 gene. In one embodiment, the nucleotide sequence encodes a TP53-NTRK1 fusion polypeptide that includes e.g., a tyrosine kinase domain or a functional fragment thereof. In yet other embodiments, the nucleic acid molecule includes a nucleotide sequence encoding the amino acid sequence (e.g., a fragment of the amino acid sequence) shown in SEQ ID NO:63 and a nucleotide sequence encoding the amino acid sequence (e.g., a fragment of the amino acid sequence) shown in SEQ ID NO:4, or a fragment of the fusion, or a sequence substantially identical thereto. In one embodiment, the encoded TP53-NTRK1 fusion polypeptide includes a tyrosine kinase domain or a functional fragment thereof.

In one embodiment, the TP53-NTRK1 fusion includes the full sequence or a fragment of the nucleotide sequence shown in SEQ ID NO:57, SEQ ID NO:59, SEQ ID NO:60, SEQ ID NO:61, SEQ ID NO: 106, SEQ ID NO: 107, SEQ ID NO: 108 and SEQ ID NO: 109, or a nucleotide sequence substantially identical thereto. In another embodiment, the TP53-NTRK11 fusion polypeptide includes the full sequence or a fragment of the amino acid sequence shown in SEQ ID NO:58, or an amino acid sequence substantially identical thereto, for example at least 70% or at least 80% or at least 90% identical or even more.

In a related aspect, the invention features nucleic acid constructs that include the TP53-NTRK1 nucleic acid molecules described herein. In certain embodiments, the nucleic acid molecules are operatively linked to a native or a heterologous regulatory sequence. Also included are vectors and host cells that include the TP53-NTRK1 nucleic acid molecules described herein, e.g., vectors and host cells suitable for producing the nucleic acid molecules and polypeptides described herein.

In a related aspect, methods of producing the nucleic acid molecules and polypeptides described herein are also described.

In another aspect, the invention features nucleic acid molecules that reduce or inhibit the expression of a nucleic acid molecule that encodes a TP53-NTRK1 fusion described herein. Examples of such nucleic acid molecules include, for example, antisense molecules, ribozymes, RNAi, triple helix molecules that hybridize to a nucleic acid encoding TP53-NTRK1, or a transcription regulatory region of TP53-NTRK1, and blocks or reduces mRNA expression of TP53-NTRK1.

### Nucleic Acid Detection and Capturing Reagents

The invention also features a nucleic acid molecule, e.g., nucleic acid fragment, suitable as probe, primer, bait or library member that includes, flanks, hybridizes to, which are useful for identifying, or are otherwise based on, the TP53-NTRK1 fusions described herein. In certain embodiments, the probe, primer or bait molecule is an oligonucleotide that allows capture, detection or isolation of a TP53-NTRK1 fusion nucleic acid molecule described herein. The oligonucleotide can comprise a nucleotide sequence substantially complementary to a fragment of the TP53-NTRK1 fusion nucleic acid molecules described herein. The sequence identity between the nucleic acid fragment, e.g., the oligonucleotide, and the target TP53-NTRK1 sequence need not be exact, so long as the sequences are sufficiently complementary to allow the capture, detection or isolation of the target sequence. In one embodiment, the nucleic acid fragment is a probe or primer that includes an oligonucleotide between about 5 and 25, e.g., between 10 and 20, or 10 and 15 nucleotides in length. In other embodiments, the nucleic acid fragment is a bait that includes an oligonucleotide between about 100 to 300 nucleotides, 130 and 230 nucleotides, or 150 and 200 nucleotides, in length.

In one embodiment, the nucleic acid fragment can be used to identify or capture, e.g., by hybridization, a TP53-NTRK1 fusion. For example, the nucleic acid fragment can be a probe, a primer, or a bait, for use in identifying or capturing, e.g., by hybridization, a TP53-NTRK11 fusion described herein. In one embodiment, the nucleic acid fragment can be useful for identifying or capturing a TP53-NTRK1 breakpoint, e.g., the nucleotide sequence of: chromosome 17 at nucleotide plus or minus 10, 20, 30, 40, 50, 60, 80, 100, 150 nucleotides and chromosome 1 at nucleotide plus or minus 10, 20, 30, 40 50, 60, 80, 100, 150 nucleotides.

In one embodiment, the nucleic acid fragment hybridizes to a nucleotide sequence within a chromosomal rearrangement that creates an in-frame fusion of intron 8 or intron 9 or intron 11 or intron 12 of TP53 with intron 8 of NTRK1. In one embodiment, the nucleic acid fragment hybridizes to a nucleotide sequence in the region In other embodiments, the nucleic acid molecules includes a nucleotide sequence in the region of a nucleotide sequence of chromosome 17 coupled to (e.g., juxtaposed to) nucleotides in the region of a nucleotide sequence Y of chromosome 17. In one embodiment, the nucleic acid fragment hybridizes to a nucleotide sequence that includes a breakpoint, e.g., the nucleotide sequence of: chromosome 17 at nucleotide plus or minus 10, 20, 30, 40 50, 60, 80, 100, 150 or more nucleotides and chromosome 1 at nucleotide plus or minus 10, 20, 30, 40 50, 60, 80, 100, 150 or more nucleotides. For example, the nucleic acid fragment can hybridize to a nucleotide sequence that includes the fusion junction between the TP53 gene and the NTRK1 gene, e.g., a nucleotide sequence that includes a portion of a nucleotide sequence within intron 8 or intron 9 or intron 11 or intron 12 of a TP53 gene and intron 8 of a NTRK1 gene.

In another embodiment, the nucleic acid fragment hybridizes to a nucleotide sequence that comprises at least 6, 12, 15, 20, 25, 50, 75, 100, 150 or more nucleotides from exon 8 or exon 9 or exon 11 or exon 12 of TP53 (e.g., from the nucleotide sequence of TP53 preceding the fusion junction with NTRK1, e.g., of the TP53 sequence shown in SEQ ID NO:62), and at least 6, 12, 15, 20, 25, 50, 75, 100, 150 or more nucleotides from exon 9 of NTRK1 (e.g., from the nucleotide sequence of NTRK1 following the fusion junction with TP53, e.g., of the NTRK1 sequence shown in SEQ ID NO:3).

The probes or primers described herein can be used, for example, for FISH detection or PCR amplification. In one exemplary embodiment where detection is based on PCR, amplification of the TP53-NTRK1 fusion junction fusion junction can be performed using a primer or a primer pair, e.g., for amplifying a sequence flanking the fusion junctions described herein, e.g., the mutations or the junction of a chromosomal rearrangement described herein, e.g., TP53-NTRK1.

In one embodiment, a pair of isolated oligonucleotide primers can amplify a region containing or adjacent to a position in the TP53-NTRK1 fusion. For example, forward primers can be designed to hybridize to a nucleotide sequence within TP53 genomic or mRNA sequence (e.g., a nucleotide sequence within exon 8 or exon 9 or exon 11 or exon 12 of TP53 of SEQ ID NO:62, and the reverse primers can be designed to hybridize to a nucleotide sequence of NTRK1 (e.g., a nucleotide sequence within exon 9 of NTRK1, of SEQ ID NO:3.

In another embodiment, the nucleic acid fragments can be used to identify. e.g., by hybridization, a TP53-NTRK1 fusion. In one embodiment, the nucleic acid fragment hybridizes to a nucleotide sequence that includes a fusion junction between the TP53 transcript and the NTRK1 transcript.

In other embodiments, the nucleic acid fragment includes a bait that comprises a nucleotide sequence that hybridizes to a TP53-NTRK1 fusion nucleic acid molecule described herein, and thereby allows the capture or isolation said nucleic acid molecule. In one embodiment, a bait is suitable for solution phase hybridization. In other embodiments, a bait includes a binding entity, e.g., an affinity tag, that allows capture and separation, e.g., by binding to a binding entity, of a hybrid formed by a bait and a nucleic acid hybridized to the bait.

In other embodiments, the nucleic acid fragment includes a library member comprising a TP53-NTRK1 nucleic acid molecule described herein. In one embodiment, the library member includes a rearrangement that results in a TP53-NTRK1 fusion described herein.

The nucleic acid fragment can be detectably labeled with, e.g., a radiolabel, a fluorescent label, a bioluminescent label, a chemiluminescent label, an enzyme label, a binding pair label, or can include an affinity tag; a tag, or identifier (e.g., an adaptor, barcode or other sequence identifier).

### TP53-NTRK1 Fusion Polypeptides

In another embodiment, the TP53-NTRK1 fusion comprises an amino acid sequence (e.g., a fragment of the amino acid sequence) shown in SEQ ID NO:63 and an amino acid sequence (e.g., a fragment of the amino acid sequence) shown in SEQ ID NO:4, or a fragment of the fusion. In one embodiment, the TP53-NTRK1 fusion comprises an amino acid sequence substantially identical to the amino acid sequence (e.g., a fragment of the amino acid sequence) shown in SEQ ID NO:63 and the amino acid sequence (e.g., a fragment of the amino acid sequence) shown in SEQ ID NO:4, or a fragment thereof. In one embodiment, the TP53-NTRK1 fusion comprises an amino acid sequence at least 70%, at least 75%, at least 80%, at least 85%, at least 90%, at least 95%, at least 99%, at least 99.5 or greater, identical to the amino acid sequence (e.g., a fragment of the amino acid sequence) shown in SEQ ID NO:63 and the amino acid sequence (e.g., a fragment of the amino acid sequence) shown in SEQ ID NO:4. In one embodiment, the TP53-NTRK1 fusion comprises a sequence containing at least 10, 20, 50, 100, 500, 600, 700, 800, 900, 1000, or more amino acids of the amino acid sequence shown in SEQ ID NO:63 and SEQ ID NO:4. In one embodiment, the TP53-NTRK1 fusion comprises an amino acid sequence containing at least 5, 10, 20, 50, 100, 500, 600, 700, 800, 900, 1000, or more contiguous amino acids of the amino acid sequence shown in 156 SEQ ID NO:63 and at least 5, 10, 20, 50, 100, 500, 600, 700, 800, 900, 1000, or more contiguous amino acids of the amino acid sequence shown in SEQ ID NO:4. In one embodiment, the 5′ TP53-3′ NTRK1 fusion polypeptide includes a receptor tyrosine kinase domain or a functional fragment thereof. In an embodiment, the 5′TP53-3′NTRK1 fusion polypeptide comprises sufficient NTRK1 and sufficient TP53 sequence such that it has kinase activity, e.g., has elevated activity.

In another aspect, the invention features a TP53-NTRK1 fusion polypeptide (e.g., a purified TP53-NTRK1 fusion polypeptide), a biologically active or antigenic fragment thereof, as well as reagents (e.g., antibody molecules that bind to a TP53-NTRK1 fusion polypeptide), methods for modulating a TP53-NTRK1 polypeptide activity and detection of a TP53-NTRK1 polypeptide.

In one embodiment, the TP53-NTRK1 fusion polypeptide has at least one biological activity. In one embodiment, at least one biological activity of the TP53-NTRK1 fusion polypeptide is reduced or inhibited by an anti-cancer drug, e.g., a TP53 inhibitor, a NTRK1 inhibitor. In one embodiment, at least one biological activity of the TP53-NTRK1 fusion polypeptide is reduced or inhibited by a NTRK1 inhibitor. In one embodiment, at least one biological activity of the TP53-NTRK1 fusion polypeptide is reduced or inhibited by a TP53 inhibitor. In one embodiment, at least one biological activity of the TP53-NTRK1 fusion polypeptide is reduced or inhibited by a NTRK1 inhibitor, e.g., lestaurtinib (CEP-701); AZ-23; indenopyrrolocarbazole 12a; oxindole 3; isothiazole 5n; thiazole 20h.

In yet other embodiments, the TP53-NTRK1 fusion polypeptide is encoded by a nucleic acid molecule described herein. In one embodiment, the TP53-NTRK1 fusion polypeptide is encoded by an in-frame fusion of intron 8 or intron 9 or intron 11 or intron 12 of TP53 with intron 8 of NTRK1 (e.g., a sequence on chromosome 17 and a sequence on chromosome 1). In another embodiment, the TP53-NTRK1 fusion polypeptide includes an amino acid sequence encoded by a nucleotide sequence comprising a fusion junction between the TP53 transcript and the NTRK1 transcript.

In certain embodiments, the TP53-NTRK1 fusion polypeptide comprises one or more of encoded exons 1-8 or exons 1-9 or exons 1-11 or exons 1-12 from TP53 and one or more of encoded exons 9-17 of NTRK1. In certain embodiments, the TP53-NTRK1 fusion polypeptide comprises at least 1, 2, 3, 4, 5, 6, 7, 8, 9, 11, 12 or more encoded exons of TP53 and at least at least 1, 2, 3, 4, 5, 6, 7, 8, 9 or more encoded exons of NTRK1. In certain embodiments, the TP53-NTRK1 fusion polypeptide comprises a fusion of encoded exon 8 or exon 9 or exon 11 or exon 12 from TP53 and encoded exon 9 from NTRK1 (or a fragment thereof). In other embodiments, the fusion comprises at least 1, 2, 3, 4, 5, 6, 7, 8, 9, 11, 12 or more encoded exons of TP53; and at least 1, 2, 3, 4, 5, 6, 7, 8, 9 or more encoded exons of NTRK1. In certain embodiments, the TP53-NTRK1 fusion polypeptide comprises encoded exons 1-8 or exons 1-9 or exons 1-11 or exons 1-12 from TP53 and exons 9-17 of NTRK1. In certain embodiments, the 5′ TP53-3′ NTRK1 fusion polypeptide comprises a fusion junction of the sequence of exon 8 or exon 9 or exon 11 or exon 12 from TP53 and the sequence of exon 9 from NTRK1.

In certain embodiments, the TP53-NTRK1 fusion comprises the amino acid sequence corresponding to exon 8 or exon 9 or exon 11 or exon 12 or a fragment thereof from TP53, and the amino acid sequence corresponding to exon 9 or a fragment thereof from NTRK1 (e.g., as shown in SEQ ID NO:63 and SEQ ID NO:4). In one embodiment, the TP53-NTRK1 fusion comprises at least 5, 10, 15, 20 or more amino acids from exon 8 or exon 9 or exon 11 or exon 12 of TP53 (e.g., from the amino acid sequence of TP53 preceding the fusion junction with NTRK1, e.g., of the TP53 sequence shown in SEQ ID NO:63), and at least 5, 10, 15, 20 or more amino acids from exon 9 of NTRK1 (e.g., from the amino acid sequence of NTRK1 following the fusion junction with TP53, e.g., of the NTRK1 sequence shown in SEQ ID NO:4).

In one embodiment, the TP53-NTRK1 fusion polypeptide includes a tyrosine kinase domain or a functional fragment thereof. In a related aspect, the invention features TP53-NTRK1 fusion polypeptide or fragments operatively linked to heterologous polypeptides to form fusion proteins.

In another embodiment, the TP53-NTRK1 fusion polypeptide or fragment is a peptide, e.g., an immunogenic peptide or protein, containing a fusion junction described herein. Such immunogenic peptides or proteins can be used to raise antibodies specific to the fusion protein. In other embodiments, such immunogenic peptides or proteins can be used for vaccine preparation. The vaccine preparation can include other components, e.g., an adjuvant.

In another aspect, the invention features antibody molecules that bind to a TP53-NTRK fusion polypeptide or fragment described herein. In embodiments, the antibody can distinguish wild type NTRK1 (or TP53) from TP53-NTRK1.

### Detection Reagents and Detection of Mutations

In another aspect, the invention features a detection reagent, e.g., a purified or an isolated preparation thereof. Detection reagents can distinguish a nucleic acid, or protein sequence, having a breakpoint, e.g., a TP53-NTRK1 breakpoint; from a reference sequence. In one embodiment, the detection reagent detects (e.g., specifically detects) a TP53-NTRK1 fusion nucleic acid or a polypeptide, e.g., distinguishes a wild type NTRK1 or another NTRK1 fusion (or TP53) from a TP53-NTRK1 nucleic acid (e.g., as described herein in SEQ ID NO:62 and SEQ ID NO:3); or a TP53-NTRK1 polypeptide (e.g., as described herein in SEQ ID NO:63 and SEQ ID NO:4).

Detection reagents, e.g., nucleic acid-based detection reagents, can be used to identify mutations in a target nucleic acid, e.g., DNA, e.g., genomic DNA or cDNA, or RNA, e.g., in a sample, e.g., a sample of nucleic acid derived from a neoplastic or tumor cell, e.g., a melanocytic neoplasm, melanoma or metastatic cell. Detection reagents, e.g., antibody-based detection reagents, can be used to identify mutations in a target protein, e.g., in a sample, e.g., a sample of protein derived from, or produced by, a neoplastic or tumor cell, e.g., a melanocytic neoplasm, melanoma or metastatic cell.

### Method of Treatment

In a related aspect, a method of inhibiting, reducing, or treating a hyperproliferative disorder, e.g., a neoplasm (including benign, pre-malignant or malignant (e.g., a cancer), in a subject is provided. The method includes administering to the subject a preselected therapeutic agent, e.g., an anti-cancer agent (e.g., a kinase inhibitor), as a single agent, or in combination, in an amount sufficient to reduce, inhibit or treat the activity or expression of TP53-NTRK1 (e.g., a TP53-NTRK1 fusion described herein), thereby inhibiting, reducing, or treating the hyperproliferative disorder in the subject.

In one embodiment, the subject treated has a TP53-NTRK1 fusion; e.g., the subject has a tumor or cancer harboring a TP53-NTRK1 fusion. In other embodiments, the subject has been previously identified as having a TP53-NTRK1 fusion. In yet other embodiments, the subject has been previously identified as being likely or unlikely to respond to treatment with a protein kinase inhibitor, e.g., a subject that has previously participated in a clinical trial. In other embodiments, the subject has been previously identified as being likely or unlikely to respond to treatment with a protein kinase inhibitor, based on the presence of the TP53-NTRK1 fusion. In one embodiment, the subject is a mammal, e.g., a human. In one embodiment, the subject has, or at risk of having a cancer at any stage of disease. In other embodiments, the subject is a patient, e.g., a cancer patient.

In certain embodiments, the neoplasm or neoplastic cell is a benign, pre-malignant, malignant (cancer) or metastasis. In certain embodiments, the cancer is a solid tumor, a soft tissue tumor, or a metastatic lesion.

In one embodiment, the neoplasm or neoplastic cell is a benign, pre-malignant, malignant (cancer) or metastasis. In certain embodiments, the cancer is a solid tumor, a soft tissue tumor, or a metastatic lesion. In one embodiment, the cancer is a carcinoma. In one embodiment, the cancer is an adenocarcinoma. In an embodiment, the cancer is a lung adenocarcinoma. In one embodiment, the cancer is chosen from a lung cancer, a pancreatic cancer, melanoma, a colorectal cancer, an esophageal-gastric cancer, a thyroid cancer, or an adenocarcinoma. In one embodiment, the cancer is a thyroid cancer. In one embodiment, the cancer is a papillary thyroid carcinoma. In one embodiment, the cancer is a lung adenocarcinoma. In other embodiment, the lung cancer is chosen from one or more of the following: non-small cell lung cancer (NSCLC), small cell lung cancer (SCLC), squamous cell carcinoma (SCC), adenocarcinoma of the lung, bronchogenic carcinoma, or a combination thereof. In one embodiment, the lung cancer is NSCLC or SCC.

In one embodiment, the anti-cancer agent is a NTRK1 inhibitor. In one embodiment, the anti-cancer agent is a TP53 inhibitor. In one embodiment, the anti-cancer agent is a kinase inhibitor. In one embodiment, the anti-cancer agent is a NTRK1 inhibitor, e.g., lestaurtinib (CEP-701); AZ-23; indenopyrrolocarboazole 12a; oxindole 3; isothiazole 5n; thiazole 20h.

### CEP89-BRAF Fusions

In one embodiment, a fusion includes an in-frame fusion of an exon of centrosomal protein 89 kDa (CEP89), e.g., one more exons of CEP89 (e.g., one or more of exons 1-16 of CEP89) or a fragment thereof, and an exon of v-raf murine sarcoma viral oncogene homolog B1 (BRAF), e.g., one or more exons of a BRAF (e.g., one or more of exons 9-18 of BRAF) or a fragment thereof. For example, the CEP89-BRAF fusion can include an in-frame fusion within an intron of CEP89 (e.g., intron 16) or a fragment thereof, with an intron of BRAF (e.g., intron 8) or a fragment thereof. In one embodiment, the fusion of the CEP89-BRAF fusion comprises the nucleotide sequence of: chromosome 19 at one or more of a nucleotide (plus or minus 10, 20, 30, 50, 60, 70, 80, 100 or more nucleotides) and chromosome 7 at one or more of a nucleotide (plus or minus 10, 20, 30, 50, 60, 70, 80, 100 or more nucleotides). In one embodiment, the CEP89-BRAF fusion is a translocation, e.g., a translocation of a portion of chromosome 19 and a portion of chromosome 7.

In certain embodiments, the CEP89-BRAF fusion is in a 5′-CEP89 to 3′-BRAF configuration (also referred to herein as “5′-CEP89-BRAF-3′).” The term “fusion” or “fusion molecule” can refer to a polypeptide or a nucleic acid fusion, depending on the context. It may include a full-length sequence of a fusion or a fragment thereof, e.g., a fusion junction (e.g., a fragment including a portion of CEP89 and a portion of BRAF, e.g., a portion of the CEP89-BRAF fusion described herein). In one embodiment, the CEP89-BRAF fusion polypeptide includes a fragment of the amino acid sequence shown in SEQ ID NO:52 and a fragment of the amino acid sequence shown in SEQ ID NO:2, or an amino acid sequence substantially identical thereto. In another embodiment, the CEP89-BRAF fusion nucleic acid includes a fragment of the nucleotide sequence shown in SEQ ID NO:51 and a fragment of the nucleotide sequence shown in SEQ ID NO: 1, or a nucleotide sequence substantially identical thereto. In one embodiment, the CEP89-BRAF fusion polypeptide comprises sufficient CEP89 and sufficient BRAF sequence such that the 5′ CEP89-3′ BRAF fusion has kinase activity, e.g., has elevated activity, e.g., tyrosine kinase activity, as compared with either wild type polypeptide. In any event, the fusion causes activation in the cells harboring it of oncogenic signaling pathways.

In certain embodiments, the CEP89-BRAF fusion comprises one or more (or all of) exons 1-16 from CEP89 and one or more (or all of) exons 9-18 of BRAF (e.g., one or more of the exons shown in SEQ ID NO:51 and SEQ ID NO: 1. In another embodiment, the CEP89-BRAF fusion comprises one or more (or all of) exons 1-16 of CEP89 and one or more (or all of) exons 9-18 of BRAF. In certain embodiments, the CEP89-BRAF fusion comprises at least 1, 2, 3, 4, 5, 6, 7, 8, 9, 10, 11, 12, 13, 14, 15, 16 or more exons (or encoded exons) from CEP89 and at least 1, 2, 3, 4, 5, 6, 7, 8, 9, 10 or more exons (or encoded exons) from BRAF (e.g., from the CEP89 and BRAF sequences shown in SEQ ID NO:51 and SEQ ID NO:52 and SEQ ID NO:1 and SEQ ID NO:2.

In certain embodiments, the CEP89-BRAF fusion comprises exons 1-16 or a fragment thereof from CEP89, and exons 9-18 or a fragment thereof from BRAF (e.g., as shown in SEQ ID NO:51 and SEQ ID NO:1). In one embodiment, the CEP89-BRAF fusion comprises at least 5, 10, 15, 20, 30, 40, 50 or more amino acids from exons 1-16 of CEP89 (e.g., from the amino acid sequence of CEP89 as shown in SEQ ID NO:52 (e.g., from the amino acid sequence of CEP89 preceding the fusion junction with BRAF, and at least 5, 10, 15, 20, 30, 40, 50 or more amino acids from exons 9-18 of BRAF (e.g., from the amino acid sequence of BRAF as shown in SEQ ID NO:2). In another embodiment, the CEP89-BRAF fusion comprises at least 6, 12, 15, 20, 25, 50, 75, 100 or more nucleotides from exons 1-16 of CEP89 (e.g., from the nucleotide sequence of CEP89 as shown in SEQ ID NO:51 (e.g., from the nucleotide sequence of CEP89 preceding the fusion junction with BRAF); and at least 6, 12, 15, 20, 25, 50, 75, 100 or more nucleotides from exons 9-18 of BRAF (e.g., from the nucleotide sequence of BRAF as shown in SEQ ID NO:1).

### CEP89-BRAF Nucleic Acid Molecules

In one aspect, the invention features a nucleic acid molecule (e.g., an isolated or purified) nucleic acid molecule that includes a fragment of a CEP89 gene and a fragment of a BRAF gene. In one embodiment, the nucleotide sequence encodes a CEP89-BRAF fusion polypeptide that includes a tyrosine kinase domain or a functional fragment thereof. In another embodiment, the nucleotide sequence encodes a fragment of the BRAF polypeptide including the amino acid sequence of SEQ ID NO:2 or a fragment thereof, or a sequence substantially identical thereto. In other embodiments, the nucleic acid molecule includes a fragment of the CEP89 gene encoding the amino acid sequence of SEQ ID NO:52 or a fragment thereof, or a sequence substantially identical thereto. In yet other embodiments, the nucleic acid molecule includes a nucleotide sequence encoding the amino acid sequence shown in SEQ ID NO:52, or a fragment thereof, and the amino acid sequence shown in SEQ ID NO:2 or a fragment thereof, or a sequence substantially identical thereto.

In one embodiment, the nucleic acid molecule includes a fusion, e.g., an in-frame fusion, between an intron of CEP89 (e.g., intron 16, or a fragment thereof), and an intron of BRAF (e.g., intron 8, or a fragment thereof). The CEP89-BRAF fusion can comprise a fusion of the nucleotide sequence of: chromosome 19 at one or more of a nucleotide (plus or minus 10, 20, 30, 50, 60, 70, 80, 100 nucleotides) and chromosome 7 at one or more of a nucleotide (plus or minus 10, 20, 30, 50, 60, 70, 80, 100 nucleotides), or a fragment thereof. In one embodiment, the CEP89-BRAF fusion comprises a fusion of the nucleotide sequence of: chromosome 19 at one or more of a nucleotide (plus or minus 10, 20, 30, 50, 60, 70, 80, 100 nucleotides) and chromosome 7 at one or more of a nucleotide (plus or minus 10, 20, 30, 50, 60, 70, 80, 100 nucleotides), or a fragment thereof.

In another embodiment, the CEP89-BRAF fusion comprises a nucleotide sequence (e.g., a fragment of a nucleotide sequence) shown in SEQ ID NO:51 and a nucleotide sequence (e.g., a fragment of a nucleotide sequence) shown in SEQ ID NO:1, or a fragment of the fusion. In one embodiment, the CEP89-BRAF fusion comprises a nucleotide sequence substantially identical to the nucleotide sequence (e.g., a fragment of a nucleotide sequence) shown in SEQ ID NO:51 and the nucleotide sequence (e.g., a fragment of a nucleotide sequence) shown SEQ ID NO: 1, or a fragment of the fusion. In one embodiment, the CEP89-BRAF fusion comprises a nucleotide sequence at least 70%, at least 75%, at least 80%, at least 85%, at least 90%, at least 95%, at least 99%, at least 99.5 or greater, identical to the nucleotide sequence (e.g., a fragment of a nucleotide sequence) shown in SEQ ID NO:51 and to the nucleotide sequence (e.g., a fragment of a nucleotide sequence) shown in SEQ ID NO:1. In one embodiment, the CEP89-BRAF fusion comprises a nucleotide sequence containing at least 25, 50, 100, 150, 200, 500, 1000, 1500, 2000, 2500, 3000, or more nucleotides of the nucleotide sequence shown in SEQ ID NO:51 and a nucleotide sequence containing at least 25, 50, 100, 150, 200, 500, 1000, 1500, 2000, 2500, 3000, or more nucleotides of the nucleotide sequence shown in SEQ ID NO: 1. In one embodiment, the CEP89-BRAF fusion comprises a nucleotide sequence containing at least 25, 50, 100, 150, 200, 500, 1000, 1500, 2000, 2500, 3000, or more contiguous nucleotides of the nucleotide sequence shown in SEQ ID NO:51 and a nucleotide sequence containing at least 25, 50, 100, 150, 200, 500, 1000, 1500, 2000, 2500, 3000, or more nucleotides of the nucleotide sequence shown in SEQ ID NO: 1.

In another embodiment, the nucleic acid molecule includes a fusion, e.g., an in-frame fusion, of at least 1, 2, 3, 4, 5, 6, 7, 8, 9, 10, 11, 12, 13, 14, 15, 16 or more exons of CEP89 or a fragment thereof (e.g., one or more of exons 1-16 of CEP89 or a fragment thereof), and at least 1, 2, 3, 4, 5, 6, 7, 8, 9, 10 or more exons of BRAF or a fragment thereof (e.g., one or more of exons 9-18 of BRAF or a fragment thereof). In yet other embodiments, the nucleic acid molecule includes a fragment the nucleotide sequence shown in SEQ ID NO:51 and a fragment of the nucleotide sequence shown in SEQ ID NO:1 or a fragment of the fusion, or a sequence substantially identical thereto.

In one embodiment, the nucleic acid molecule is complementary to at least a portion of a nucleotide sequence disclosed herein, e.g., is capable of hybridizing under a stringency condition described herein to SEQ ID NO:51 and/or SEQ ID NO: 1, or a fragment thereof. In yet another embodiment, the nucleic acid molecule hybridizes to a nucleotide sequence that is complementary to at least a portion of a nucleotide sequence disclosed herein, e.g., is capable of hybridizing under a stringency condition to a nucleotide sequence complementary to SEQ ID NO:51 and/or SEQ ID NO: 1, or a fragment thereof. The nucleotide sequence of a cDNA encoding an exemplary 5′ CEP89-3′ BRAF fusion is shown in at least exon 16 (e.g., exons 1-16) of SEQ ID NO:51 and at least exon 9 (e.g., exons 9-18) of SEQ ID NO: 1, and the predicted amino acid sequence is shown in the corresponding encoded exons of SEQ ID NO:52 and the corresponding encoded exons of SEQ ID NO:2, respectively.

In an embodiment the CEP89-BRAF nucleic acid molecule comprises sufficient CEP89 and sufficient BRAF sequence such that the encoded 5′ CEP89-3′ BRAF fusion has kinase activity, e.g., has elevated activity. In certain embodiments, the 5′ CEP89-3′ BRAF fusion comprises exons 1-16 from CEP89 and exons 9-18 from BRAF. In certain embodiments, the CEP89-BRAF fusion comprises at least 1, 2, 3, 4, 5, 6, 7, 8, 9, 10, 11, 12, 13, 14, 15, 16 or more exons from CEP89 and at least at least 1, 2, 3, 4, 5, 6, 7, 8, 9, 10 or more exons of BRAF. In certain embodiments, the CEP89-BRAF fusion comprises a fusion of exon 16 from CEP89 and exon 9 from BRAF. In another embodiment, the CEP89-BRAF fusion comprises 1, 2, 3, 4, 5, 6, 7, 8, 9, 10, 11, 12, 13, 14, 15, 16 or more exons of CEP89; and at least 1, 2, 3, 4, 5, 6, 7, 8, 9, 10 or more exons of BRAF.

In one embodiment, the nucleic acid molecule includes a nucleotide sequence that has an in-frame fusion of intron 16 of CEP89 (e.g., NM 032816) with intron 8 of BRAF (e.g., NM 004333). In another embodiment, the nucleic acid molecule includes a nucleotide sequence that includes a breakpoint. For example, the nucleic acid molecule includes a nucleotide sequence that includes the fusion junction between the CEP89 gene and the BRAF gene, e.g., the breakpoint between intron 16 of CEP89 and intron 8 of BRAF. In other embodiments, the nucleic acid molecules includes a nucleotide sequence of one or more of nucleotide of chromosome 19 coupled to (e.g., directly or indirectly juxtaposed to) one or more of nucleotide of chromosome 7. In one embodiment, the nucleic acid molecule includes the nucleotide sequence of: chromosome 19 at one or more of a nucleotide plus or minus 10, 20, 30, 40 50, 60, 80, 100, 150 nucleotides and chromosome 7 at one or more of a nucleotide plus or minus 10, 20, 30, 40 50, 60, 80, 100, 150 nucleotides (corresponding to the breakpoint of a CEP89-BRAF fusion), or a fragment thereof, or a sequence substantially identical thereto. In one embodiment, the nucleic acid molecule is complementary to at least a portion of a nucleotide sequence disclosed herein, e.g., is capable of hybridizing under a stringency condition described herein to SEQ ID NO:51 and/or SEQ ID NO: 11 or a fragment thereof. In yet other embodiment, the nucleic acid molecule hybridizes to a nucleotide sequence that is complementary to at least a portion of a nucleotide sequence disclosed herein, e.g., is capable of hybridizing under a stringency condition described herein to a nucleotide sequence complementary to SEQ ID NO:51 or SEQ ID NO:1 or a fragment thereof.

In another embodiment, the CEP89-BRAF fusion nucleic acid comprises at least 6, 12, 15, 20, 25, 50, 75, 100 or more nucleotides from exon 16 of CEP89 (e.g., from the nucleotide sequence of CEP89 preceding the fusion junction with BRAF, e.g., of the CEP89 sequence shown in SEQ ID NO:51), and at least 6, 12, 15, 20, 25, 50, 75, 100 or more nucleotides from exon 9 of BRAF (e.g., from the nucleotide sequence of BRAF following the fusion junction with CEP89, e.g., of the BRAF sequence shown in SEQ ID NO:1).

In other embodiments, the nucleic acid molecule includes a nucleotide sequence encoding a CEP89-BRAF fusion polypeptide that includes a fragment of a CEP89 gene and a fragment of a BRAF gene. In one embodiment, the nucleotide sequence encodes a CEP89-BRAF fusion polypeptide that includes e.g., a tyrosine kinase domain or a functional fragment thereof. In yet other embodiments, the nucleic acid molecule includes a nucleotide sequence encoding the amino acid sequence (e.g., a fragment of the amino acid sequence) shown in SEQ ID NO:52 and a nucleotide sequence encoding the amino acid sequence (e.g., a fragment of the amino acid sequence) shown in SEQ ID NO:2, or a fragment of the fusion, or a sequence substantially identical thereto. In one embodiment, the encoded CEP89-BRAF fusion polypeptide includes a tyrosine kinase domain or a functional fragment thereof.

In one embodiment, the CEP89-BRAF fusion includes the full sequence or a fragment of the nucleotide sequence shown in SEQ ID NO:49 and SEQ ID NO:104, or a nucleotide sequence substantially identical thereto. In another embodiment, the CEP89-BRAF fusion polypeptide includes the full sequence or a fragment of the amino acid sequence shown in SEQ ID NO:50 and SEQ ID NO: 105, or an amino acid sequence substantially identical thereto, for example at least 70% or at least 80% or at least 90% identical or even more.

In a related aspect, the invention features nucleic acid constructs that include the CEP89-BRAF nucleic acid molecules described herein. In certain embodiments, the nucleic acid molecules are operatively linked to a native or a heterologous regulatory sequence. Also included are vectors and host cells that include the CEP89-BRAF nucleic acid molecules described herein, e.g., vectors and host cells suitable for producing the nucleic acid molecules and polypeptides described herein.

In a related aspect, methods of producing the nucleic acid molecules and polypeptides described herein are also described.

In another aspect, the invention features nucleic acid molecules that reduce or inhibit the expression of a nucleic acid molecule that encodes a CEP89-BRAF fusion described herein. Examples of such nucleic acid molecules include, for example, antisense molecules, ribozymes, RNAi, triple helix molecules that hybridize to a nucleic acid encoding CEP89-BRAF, or a transcription regulatory region of CEP89-BRAF, and blocks or reduces mRNA expression of CEP89-BRAF.

### Nucleic Acid Detection and Capturing Reagents

The invention also features a nucleic acid molecule, e.g., nucleic acid fragment, suitable as probe, primer, bait or library member that includes, flanks, hybridizes to, which are useful for identifying, or are otherwise based on, the CEP89-BRAF fusions described herein. In certain embodiments, the probe, primer or bait molecule is an oligonucleotide that allows capture, detection or isolation of a CEP89-BRAF fusion nucleic acid molecule described herein. The oligonucleotide can comprise a nucleotide sequence substantially complementary to a fragment of the CEP89-BRAF fusion nucleic acid molecules described herein. The sequence identity between the nucleic acid fragment, e.g., the oligonucleotide, and the target CEP89-BRAF sequence need not be exact, so long as the sequences are sufficiently complementary to allow the capture, detection or isolation of the target sequence. In one embodiment, the nucleic acid fragment is a probe or primer that includes an oligonucleotide between about 5 and 25, e.g., between 10 and 20, or 10 and 15 nucleotides in length. In other embodiments, the nucleic acid fragment is a bait that includes an oligonucleotide between about 100 to 300 nucleotides, 130 and 230 nucleotides, or 150 and 200 nucleotides, in length.

In one embodiment, the nucleic acid fragment can be used to identify or capture, e.g., by hybridization, a CEP89-BRAF fusion. For example, the nucleic acid fragment can be a probe, a primer, or a bait, for use in identifying or capturing, e.g., by hybridization, a CEP89-BRAF fusion described herein. In one embodiment, the nucleic acid fragment can be useful for identifying or capturing a CEP89-BRAF breakpoint, e.g., the nucleotide sequence of: chromosome 19 at nucleotide plus or minus 10, 20, 30, 40, 50, 60, 80, 100, 150 nucleotides and chromosome 7 at nucleotide plus or minus 10, 20, 30, 40 50, 60, 80, 100, 150 nucleotides.

In one embodiment, the nucleic acid fragment hybridizes to a nucleotide sequence within a chromosomal rearrangement that creates an in-frame fusion of intron 16 of CEP89 with intron 8 of BRAF. In one embodiment, the nucleic acid fragment hybridizes to a nucleotide sequence in the region In other embodiments, the nucleic acid molecules includes a nucleotide sequence in the region of a nucleotide sequence of chromosome 19 coupled to (e.g., juxtaposed to) nucleotides in the region of a nucleotide sequence Y of chromosome 17. In one embodiment, the nucleic acid fragment hybridizes to a nucleotide sequence that includes a breakpoint, e.g., the nucleotide sequence of: chromosome 19 at nucleotide plus or minus 10, 20, 30, 40 50, 60, 80, 100, 150 or more nucleotides and chromosome 7 at nucleotide plus or minus 10, 20, 30, 40 50, 60, 80, 100, 150 or more nucleotides. For example, the nucleic acid fragment can hybridize to a nucleotide sequence that includes the fusion junction between the CEP89 gene and the BRAF gene, e.g., a nucleotide sequence that includes a portion of a nucleotide sequence within intron 16 of a CEP89 gene and intron 8 of a BRAF gene.

In another embodiment, the nucleic acid fragment hybridizes to a nucleotide sequence that comprises at least 6, 12, 15, 20, 25, 50, 75, 100, 150 or more nucleotides from exon 16 of CEP89 (e.g., from the nucleotide sequence of CEP89 preceding the fusion junction with BRAF, e.g., of the CEP89 sequence shown in SEQ ID NO:51), and at least 6, 12, 15, 20, 25, 50, 75, 100, 150 or more nucleotides from exon 9 of BRAF (e.g., from the nucleotide sequence of BRAF following the fusion junction with CEP89, e.g., of the BRAF sequence shown in SEQ ID NO: 1).

The probes or primers described herein can be used, for example, for FISH detection or PCR amplification. In one exemplary embodiment where detection is based on PCR, amplification of the CEP89-BRAF fusion junction fusion junction can be performed using a primer or a primer pair, e.g., for amplifying a sequence flanking the fusion junctions described herein, e.g., the mutations or the junction of a chromosomal rearrangement described herein, e.g., CEP89-BRAF.

In one embodiment, a pair of isolated oligonucleotide primers can amplify a region containing or adjacent to a position in the CEP89-BRAF fusion. For example, forward primers can be designed to hybridize to a nucleotide sequence within CEP89 genomic or mRNA sequence (e.g., a nucleotide sequence within exon 16 of CEP89 of SEQ ID NO:51, and the reverse primers can be designed to hybridize to a nucleotide sequence of BRAF (e.g., a nucleotide sequence within exon 9 of BRAF, of SEQ ID NO:1.

In another embodiment, the nucleic acid fragments can be used to identify, e.g., by hybridization, a CEP89-BRAF fusion. In one embodiment, the nucleic acid fragment hybridizes to a nucleotide sequence that includes a fusion junction between the CEP89 transcript and the BRAF transcript.

In other embodiments, the nucleic acid fragment includes a bait that comprises a nucleotide sequence that hybridizes to a CEP89-BRAF fusion nucleic acid molecule described herein, and thereby allows the capture or isolation said nucleic acid molecule. In one embodiment, a bait is suitable for solution phase hybridization. In other embodiments, a bait includes a binding entity, e.g., an affinity tag, that allows capture and separation, e.g., by binding to a binding entity, of a hybrid formed by a bait and a nucleic acid hybridized to the bait.

In other embodiments, the nucleic acid fragment includes a library member comprising a CEP89-BRAF nucleic acid molecule described herein. In one embodiment, the library member includes a rearrangement that results in a CEP89-BRAF fusion described herein.

The nucleic acid fragment can be detectably labeled with, e.g., a radiolabel, a fluorescent label, a bioluminescent label, a chemiluminescent label, an enzyme label, a binding pair label, or can include an affinity tag; a tag, or identifier (e.g., an adaptor, barcode or other sequence identifier).

### CEP89-BRAF Fusion Polypeptides

In another embodiment, the CEP89-BRAF fusion comprises an amino acid sequence (e.g., a fragment of the amino acid sequence) shown in SEQ ID NO:52 and an amino acid sequence (e.g., a fragment of the amino acid sequence) shown in SEQ ID NO:2, or a fragment of the fusion. In one embodiment, the CEP89-BRAF fusion comprises an amino acid sequence substantially identical to the amino acid sequence (e.g., a fragment of the amino acid sequence) shown in SEQ ID NO:52 and the amino acid sequence (e.g., a fragment of the amino acid sequence) shown in SEQ ID NO:2, or a fragment thereof. In one embodiment, the CEP89-BRAF fusion comprises an amino acid sequence at least 70%, at least 75%, at least 80%, at least 85%, at least 90%, at least 95%, at least 99%, at least 99.5 or greater, identical to the amino acid sequence (e.g., a fragment of the amino acid sequence) shown in 142 SEQ ID NO:52 and the amino acid sequence (e.g., a fragment of the amino acid sequence) shown in SEQ ID NO:2. In one embodiment, the CEP89-BRAF fusion comprises a sequence containing at least 10, 20, 50, 100, 500, 600, 700, 800, 900, 1000, or more amino acids of the amino acid sequence shown in SEQ ID NO:52 and SEQ ID NO:2. In one embodiment, the CEP89-BRAF fusion comprises an amino acid sequence containing at least 5, 10, 20, 50, 100, 500, 600, 700, 800, 900, 1000, or more contiguous amino acids of the amino acid sequence shown in SEQ ID NO:52 and at least 5, 10, 20, 50, 100, 500, 600, 700, 800, 900, 1000, or more contiguous amino acids of the amino acid sequence shown in SEQ ID NO:2. In one embodiment, the 5′ CEP89-3′ BRAF fusion polypeptide includes a receptor tyrosine kinase domain or a functional fragment thereof. In an embodiment, the 5′CEP89-3′BRAF fusion polypeptide comprises sufficient BRAF and sufficient CEP89 sequence such that it has kinase activity, e.g., has elevated activity.

In another aspect, the invention features a CEP89-BRAF fusion polypeptide (e.g., a purified CEP89-BRAF fusion polypeptide), a biologically active or antigenic fragment thereof, as well as reagents (e.g., antibody molecules that bind to a CEP89-BRAF fusion polypeptide), methods for modulating a CEP89-BRAF polypeptide activity and detection of a CEP89-BRAF polypeptide.

In one embodiment, the CEP89-BRAF fusion polypeptide has at least one biological activity. In one embodiment, at least one biological activity of the CEP89-BRAF fusion polypeptide is reduced or inhibited by an anti-cancer drug, e.g., a CEP89 inhibitor, a BRAF inhibitor. In one embodiment, at least one biological activity of the CEP89-BRAF fusion polypeptide is reduced or inhibited by a BRAF inhibitor. In one embodiment, at least one biological activity of the CEP89-BRAF fusion polypeptide is reduced or inhibited by a CEP89 inhibitor. In one embodiment, at least one biological activity of the CEP89-BRAF fusion polypeptide is reduced or inhibited by a BRAF inhibitor, e.g., vemurafenib (also known as RG7204; or PLX4032; or Zelboraf); GDC-0879; PLX-4702; AZ628; dabrafenib (GSK2118346A); or Sorafenib Tosylate.

In yet other embodiments, the CEP89-BRAF fusion polypeptide is encoded by a nucleic acid molecule described herein. In one embodiment, the CEP89-BRAF fusion polypeptide is encoded by an in-frame fusion of intron 16 of CEP89 with intron 8 of BRAF (e.g., a sequence on chromosome 19 and a sequence on chromosome 7). In another embodiment, the CEP89-BRAF fusion polypeptide includes an amino acid sequence encoded by a nucleotide sequence comprising a fusion junction between the CEP89 transcript and the BRAF transcript.

In certain embodiments, the CEP89-BRAF fusion polypeptide comprises one or more of encoded exons 1-16 from CEP89 and one or more of encoded exons 9-18 of BRAF. In certain embodiments, the CEP89-BRAF fusion polypeptide comprises at least 1, 2, 3, 4, 5, 6, 7, 8, 9, 10, 11, 12, 13, 14, 15, 16 or more encoded exons of CEP89 and at least at least 1, 2, 3, 4, 5, 6, 7, 8, 9, 10 or more encoded exons of BRAF. In certain embodiments, the CEP89-BRAF fusion polypeptide comprises a fusion of encoded exon 16 from CEP89 and encoded exon 9 from BRAF (or a fragment thereof). In other embodiments, the fusion comprises at least 1, 2, 3, 4, 5, 6, 7, 8, 9, 10, 11, 12, 13, 14, 15, 16 or more encoded exons of CEP89; and at least 1, 2, 3, 4, 5, 6, 7, 8, 9, 10 or more encoded exons of BRAF. In certain embodiments, the CEP89-BRAF fusion polypeptide comprises encoded exons 1-16 from CEP89 and exons 9-18 of BRAF. In certain embodiments, the 5′ CEP89-3′ BRAF fusion polypeptide comprises a fusion junction of the sequence of exon 16 from CEP89 and the sequence of exon 9 from BRAF.

In certain embodiments, the CEP89-BRAF fusion comprises the amino acid sequence corresponding to exon 16 or a fragment thereof from CEP89, and the amino acid sequence corresponding to exon 9 or a fragment thereof from BRAF (e.g., as shown in SEQ ID NO:52 and SEQ ID NO:2). In one embodiment, the CEP89-BRAF fusion comprises at least 5, 10, 15, 20 or more amino acids from exon 16 of CEP89 (e.g., from the amino acid sequence of CEP89 preceding the fusion junction with BRAF, e.g., of the CEP89 sequence shown in SEQ ID NO:52), and at least 5, 10, 15, 20 or more amino acids from exon 9 of BRAF (e.g., from the amino acid sequence of BRAF following the fusion junction with CEP89, e.g., of the BRAF sequence shown in SEQ ID NO:2).

In one embodiment, the CEP89-BRAF fusion polypeptide includes a tyrosine kinase domain or a functional fragment thereof. In a related aspect, the invention features CEP89-BRAF fusion polypeptide or fragments operatively linked to heterologous polypeptides to form fusion proteins.

In another embodiment, the CEP89-BRAF fusion polypeptide or fragment is a peptide, e.g., an immunogenic peptide or protein containing a fusion junction described herein. Such immunogenic peptides or proteins can be used to raise antibodies specific to the fusion protein. In other embodiments, such immunogenic peptides or proteins can be used for vaccine preparation. The vaccine preparation can include other components, e.g., an adjuvant.

In another aspect, the invention features antibody molecules that bind to a CEP89-BRAF fusion polypeptide or fragment described herein. In embodiments, the antibody can distinguish wild type BRAF (or CEP89) from CEP89-BRAF.

### Detection Reagents and Detection of Mutations

In another aspect, the invention features a detection reagent, e.g., a purified or an isolated preparation thereof. Detection reagents can distinguish a nucleic acid, or protein sequence, having a breakpoint, e.g., a CEP89-BRAF breakpoint; from a reference sequence. In one embodiment, the detection reagent detects (e.g., specifically detects) a CEP89-BRAF fusion nucleic acid or a polypeptide, e.g., distinguishes a wild type BRAF or another BRAF fusion (or CEP89) from a CEP89-BRAF nucleic acid (e.g., as described herein in SEQ ID NO:51 and SEQ ID NO: 1); or a CEP89-BRAF polypeptide (e.g., as described herein in SEQ ID NO:52 and SEQ ID NO:2).

Detection reagents, e.g., nucleic acid-based detection reagents, can be used to identify mutations in a target nucleic acid, e.g., DNA, e.g., genomic DNA or cDNA, or RNA. e.g., in a sample, e.g., a sample of nucleic acid derived from a neoplastic or tumor cell, e.g., a melanocytic neoplasm, melanoma or metastatic cell. Detection reagents, e.g., antibody-based detection reagents, can be used to identify mutations in a target protein, e.g., in a sample, e.g., a sample of protein derived from, or produced by, a neoplastic or tumor cell, e.g., a melanocytic neoplasm, melanoma or metastatic cell.

### Method of Treatment

In a related aspect, a method of inhibiting, reducing, or treating a hyperproliferative disorder, e.g., a neoplasm (including benign, pre-malignant or malignant (e.g., a cancer), in a subject is provided. The method includes administering to the subject a preselected therapeutic agent, e.g., an anti-cancer agent (e.g., a kinase inhibitor), as a single agent, or in combination, in an amount sufficient to reduce, inhibit or treat the activity or expression of CEP89-BRAF (e.g., a CEP89-BRAF fusion described herein), thereby inhibiting, reducing, or treating the hyperproliferative disorder in the subject.

In one embodiment, the subject treated has a CEP89-BRAF fusion; e.g., the subject has a tumor or cancer harboring a CEP89-BRAF fusion. In other embodiments, the subject has been previously identified as having a CEP89-BRAF fusion. In yet other embodiments, the subject has been previously identified as being likely or unlikely to respond to treatment with a protein kinase inhibitor, e.g., a subject that has previously participated in a clinical trial. In other embodiments, the subject has been previously identified as being likely or unlikely to respond to treatment with a protein kinase inhibitor, based on the presence of the CEP89-BRAF fusion. In one embodiment, the subject is a mammal, e.g., a human. In one embodiment, the subject has, or at risk of having a cancer at any stage of disease. In other embodiments, the subject is a patient, e.g., a cancer patient.

In certain embodiments, the neoplasm or neoplastic cell is a benign, pre-malignant, malignant (cancer) or metastasis. In certain embodiments, the cancer is a solid tumor, a soft tissue tumor, or a metastatic lesion.

In one embodiment, the neoplasm or neoplastic cell is a benign, pre-malignant, malignant (cancer) or metastasis. In certain embodiments, the cancer is a solid tumor, a soft tissue tumor, or a metastatic lesion. In one embodiment, the cancer is a carcinoma. In one embodiment the cancer is a papillary thyroid carcinoma. In one embodiment the cancer is a pilocytic astrocytomas. In one embodiment, the cancer is a melanocytic tumor. In one embodiment, the cancer is an adenocarcinoma. In an embodiment, the cancer is a lung adenocarcinoma. In one embodiment, the cancer is chosen from a lung cancer, a pancreatic cancer, melanoma, a colorectal cancer, an esophageal-gastric cancer, a thyroid cancer, or an adenocarcinoma. In one embodiment, the cancer is a lung adenocarcinoma. In other embodiment, the lung cancer is chosen from one or more of the following: non-small cell lung cancer (NSCLC), small cell lung cancer (SCLC), squamous cell carcinoma (SCC), adenocarcinoma of the lung, bronchogenic carcinoma, or a combination thereof. In one embodiment, the lung cancer is NSCLC or SCC.

In one embodiment, the anti-cancer agent is a BRAF inhibitor. In one embodiment, the anti-cancer agent is a CEP89 inhibitor. In one embodiment, the anti-cancer agent is a kinase inhibitor. In one embodiment, the anti-cancer agent is a BRAF inhibitor, e.g., vemurafenib (also known as RG7204; or PLX4032; or Zelboraf); GDC-0879; PLX-4702; AZ628; dabrafenib (GSK2118346A); or Sorafenib Tosylate.

### HLA-A-ROS1 Fusions

In one embodiment, a fusion includes an in-frame fusion of an exon of human leukocyte antigens A (HLA-A), e.g., one more exons of HLA-A (e.g., one or more of exons 1-7 of HLA-A) or a fragment thereof, and an exon of C-Ros oncogene 1 (ROS1. e.g., one or more exons of a ROS1 (such as, one or more of exons 34-43 of ROS1) or a fragment thereof. For example, the HLA-A-ROS1 fusion can include an in-frame fusion within an intron of HLA-A (e.g., intron 7 or a fragment thereof, with an intron of ROS1 (for example, intron 33) or a fragment thereof. In one embodiment, the fusion of the HLA-A-ROS1 fusion comprises the nucleotide sequence of: chromosome 6 at one or more of a nucleotide (plus or minus 10, 20, 30, 50, 60, 70, 80, 100 or more nucleotides) and chromosome 6 at one or more of a nucleotide (plus or minus 10, 20, 30, 50, 60, 70, 80, 100 or more nucleotides). In one embodiment, the HLA-A-ROS1 fusion is a translocation. e.g., a translocation of a portion of chromosome 6 and a portion of chromosome 6.

In certain embodiments, the HLA-A-ROS1 fusion is in a 5′-HLA-A to 3′-ROS1 configuration (also referred to herein as “5′-HLA-A-ROS1-3′).” The term “fusion” or “fusion molecule” can refer to a polypeptide or a nucleic acid fusion, depending on the context. It may include a full-length sequence of a fusion or a fragment thereof, e.g., a fusion junction (e.g., a fragment including a portion of HLA-A and a portion of ROS1, e.g., a portion of the HLA-A-ROS fusion described herein). In one embodiment, the HLA-A-ROS1 fusion polypeptide includes a fragment of the amino acid sequence shown in SEQ ID NO:38 and a fragment of the amino acid sequence shown in SEQ ID NO:12, or an amino acid sequence substantially identical thereto. In another embodiment, the HLA-A-ROS1 fusion nucleic acid includes a fragment of the nucleotide sequence shown in SEQ ID NO:37 and a fragment of the nucleotide sequence shown in SEQ ID NO: 1, or a nucleotide sequence substantially identical thereto. In one embodiment, the HLA-A-ROS1 fusion polypeptide comprises sufficient HLA-A and sufficient ROS1 sequence such that the 5′ HLA-A-3′ ROS1 fusion has kinase activity, e.g., has elevated activity, e.g., tyrosine kinase activity. In any event, the fusion causes activation in the cells harboring it of oncogenic signaling pathways.

In certain embodiments, the HLA-A-ROS1 fusion comprises one or more (or all of) exons 1-7 from HLA-A and one or more (or all of) exons 34-43 of ROS1 (e.g., one or more of the exons shown in SEQ ID NO:37 and SEQ ID NO: 11. In another embodiment, the HLA-A-ROS1 fusion comprises one or more (or all of) exons 1-7 of HLA-A and one or more (or all of) exons 34-43 of ROS1. In certain embodiments, the HLA-A-ROS1 fusion comprises at least 1, 2, 3, 4, 5, 6, 7 or more exons (or encoded exons) from HLA-A and at least 1, 2, 3, 4, 5, 6, 7, 8, 9, 10 or more exons (or encoded exons) from ROS1 (e.g., from the HLA-A and ROS1 sequences shown in SEQ ID NO:37 and SEQ ID NO:38 and SEQ ID NO: 11 and SEQ ID NO:12.

In certain embodiments, the HLA-A-ROS1 fusion comprises exons 1-7 or a fragment thereof from HLA-A, and exons 34-43 or a fragment thereof from ROS1 (e.g., as shown in SEQ ID NO:37 and SEQ ID NO:11. In one embodiment, the HLA-A-ROS1 fusion comprises at least 5, 10, 15, 20, 30, 40, 50 or more amino acids from exons 1-7 of HLA-A (e.g., from the amino acid sequence of HLA-A as shown in SEQ ID NO:38 (e.g., from the amino acid sequence of HLA-A preceding the fusion junction with ROS1, and at least 5, 10, 15, 20, 30, 40, 50 or more amino acids from exons 34-43 of ROS1 (e.g., from the amino acid sequence of ROS1 as shown in SEQ ID NO:12. In another embodiment, the HLA-A-ROS1 fusion comprises at least 6, 12, 15, 20, 25, 50, 75, 100 or more nucleotides from exons 1-7 of HLA-A (e.g., from the nucleotide sequence of HLA-A as shown in SEQ ID NO:37 (e.g., from the nucleotide sequence of HLA-A preceding the fusion junction with ROS1; and at least 6, 12, 15, 20, 25, 50, 75, 100 or more nucleotides from exons 34-43 of ROS1 (e.g., from the nucleotide sequence of ROS1 as shown in SEQ ID NO: 11.

### HLA-A-ROS1 Nucleic Acid Molecules

In one aspect, the invention features a nucleic acid molecule (e.g., an isolated or purified) nucleic acid molecule that includes a fragment of a HLA-A gene and a fragment of a ROS1 gene. In one embodiment, the nucleotide sequence encodes a HLA-A-ROS1 fusion polypeptide that includes a tyrosine kinase domain or a functional fragment thereof. In another embodiment, the nucleotide sequence encodes a fragment of the ROS1 polypeptide including the amino acid sequence of SEQ ID NO: 12 or a fragment thereof, or a sequence substantially identical thereto. In other embodiments, the nucleic acid molecule includes a fragment of the HLA-A gene encoding the amino acid sequence of SEQ ID NO:38 or a fragment thereof, or a sequence substantially identical thereto. In yet other embodiments, the nucleic acid molecule includes a nucleotide sequence encoding the amino acid sequence shown in SEQ ID NO:38, or a fragment thereof, and the amino acid sequence shown in SEQ ID NO: 12 or a fragment thereof, or a sequence substantially identical thereto.

In one embodiment, the nucleic acid molecule includes a fusion, e.g., an in-frame fusion, between an intron of HLA-A (e.g., intron 7, or a fragment thereof), and an intron of ROS1 (e.g., intron 33, or a fragment thereof). The HLA-A-ROS1 fusion can comprise a fusion of the nucleotide sequence of: chromosome 6 at one or more of a nucleotide (plus or minus 10, 20, 30, 50, 60, 70, 80, 100 nucleotides) and chromosome 6 at one or more of a nucleotide (plus or minus 10, 20, 30, 50, 60, 70, 80, 100 nucleotides), or a fragment thereof. In one embodiment, the HLA-A-ROS1 fusion comprises a fusion of the nucleotide sequence of: chromosome 6 at one or more of a nucleotide (plus or minus 10, 20, 30, 50, 60, 70, 80, 100 nucleotides) and chromosome 6 at one or more of a nucleotide (plus or minus 10, 20, 30, 50, 60, 70, 80, 100 nucleotides), or a fragment thereof.

In another embodiment, the HLA-A-ROS1 fusion comprises a nucleotide sequence (e.g., a fragment of a nucleotide sequence) shown in SEQ ID NO:37 and a nucleotide sequence (e.g., a fragment of a nucleotide sequence) shown in SEQ ID NO: 11, or a fragment of the fusion. In one embodiment, the HLA-A-ROS1 fusion comprises a nucleotide sequence substantially identical to the nucleotide sequence (e.g., a fragment of a nucleotide sequence) shown in SEQ ID NO:37 and the nucleotide sequence (e.g., a fragment of a nucleotide sequence) shown SEQ ID NO: 11, or a fragment of the fusion. In one embodiment, the HLA-A-ROS1 fusion comprises a nucleotide sequence at least 70%, at least 75%, at least 80%, at least 85%, at least 90%, at least 95%, at least 99%, at least 99.5 or greater, identical to the nucleotide sequence (e.g., a fragment of a nucleotide sequence) shown in SEQ ID NO:37 and to the nucleotide sequence (e.g., a fragment of a nucleotide sequence) shown in SEQ ID NO: 11. In one embodiment, the HLA-A-ROS1 fusion comprises a nucleotide sequence containing at least 25, 50, 100, 150, 200, 500, 1000, 1500, 2000, 2500, 3000, or more nucleotides of the nucleotide sequence shown in SEQ ID NO:37 and a nucleotide sequence containing at least 25, 50, 100, 150, 200, 500, 1000, 1500, 2000, 2500, 3000, or more nucleotides of the nucleotide sequence shown in SEQ ID NO:11. In one embodiment, the HLA-A-ROS1 fusion comprises a nucleotide sequence containing at least 25, 50, 100, 150, 200, 500, 1000, 1500, 2000, 2500, 3000, or more contiguous nucleotides of the nucleotide sequence shown in SEQ ID NO:37 and a nucleotide sequence containing at least 25, 50, 100, 150, 200, 500, 1000, 1500, 2000, 2500, 3000, or more nucleotides of the nucleotide sequence shown in SEQ ID NO:11.

In another embodiment, the nucleic acid molecule includes a fusion, e.g., an in-frame fusion, of at least 1, 2, 3, 4, 5, 6, 7 or more exons of HLA-A or a fragment thereof (e.g., one or more of exons 1-7 of HLA-A or a fragment thereof), and at least 1, 2, 3, 4, 5, 6, 7, 8, 9, 10 or more exons of ROS1 or a fragment thereof (e.g., one or more of exons 34-43 of ROS1 or a fragment thereof). In yet other embodiments, the nucleic acid molecule includes a fragment the nucleotide sequence shown in SEQ ID NO:37 and a fragment of the nucleotide sequence shown in SEQ ID NO: 11 or a fragment of the fusion, or a sequence substantially identical thereto.

In one embodiment, the nucleic acid molecule is complementary to at least a portion of a nucleotide sequence disclosed herein, e.g., is capable of hybridizing under a stringency condition described herein to SEQ ID NO:37 and/or SEQ ID NO: 11, or a fragment thereof. In yet another embodiment, the nucleic acid molecule hybridizes to a nucleotide sequence that is complementary to at least a portion of a nucleotide sequence disclosed herein, e.g., is capable of hybridizing under a stringency condition to a nucleotide sequence complementary to SEQ ID NO:37 and/or SEQ ID NO: 11, or a fragment thereof. The nucleotide sequence of a cDNA encoding an exemplary 5′ HLA-A-3′ ROS1 fusion is shown in at least exon 7 (e.g., exons 1-7 of SEQ ID NO:37 and at least exon 34 (e.g., exons 34-43 of SEQ ID NO: 11, and the predicted amino acid sequence is shown in the corresponding encoded exons of SEQ ID NO: 38 and the corresponding encoded exons of SEQ ID NO: 12, respectively.

In an embodiment the HLA-A-ROS1 nucleic acid molecule comprises sufficient HLA-A and sufficient ROS1 sequence such that the encoded 5′ HLA-A-3′ ROS1 fusion has kinase activity, e.g., has elevated activity. In certain embodiments, the 5′ HLA-A-3′ ROS1 fusion comprises exons 1-7 from HLA-A and exons 36-43 from ROS1. In certain embodiments, the HLA-A-ROS1 fusion comprises at least 1, 2, 3, 4, 5, 6, 7 or more exons from HLA-A and at least at least 1, 2, 3, 4, 5, 6, 7, 8, 9, 10 or more exons of ROS1. In certain embodiments, the HLA-A-ROS1 fusion comprises a fusion of exon 7 from HLA-A and exon 34 from ROS1. In another embodiment, the HLA-A-ROS1 fusion comprises 1, 2, 3, 4, 5, 6, 7 or more exons of HLA-A; and at least 1, 2, 3, 4, 5, 6, 7, 8, 9, 10 or more exons of ROS1.

In one embodiment, the nucleic acid molecule includes a nucleotide sequence that has an in-frame fusion of intron 7 of HLA-A (e.g., NM_{—}002116 with intron 34 of ROS1 (e.g., NM_{—}002944. In another embodiment, the nucleic acid molecule includes a nucleotide sequence that includes a breakpoint. For example, the nucleic acid molecule includes a nucleotide sequence that includes the fusion junction between the HLA-A gene and the ROS1 gene. e.g., the breakpoint between intron 7 of HLA-A and intron 33 of ROS1. In other embodiments, the nucleic acid molecules includes a nucleotide sequence of one or more of nucleotide of chromosome 6 coupled to (e.g., directly or indirectly juxtaposed to) one or more of nucleotide of chromosome 6. In one embodiment, the nucleic acid molecule includes the nucleotide sequence of: chromosome 6 at one or more of a nucleotide plus or minus 10, 20, 30, 40 50, 60, 80, 100, 150 nucleotides and chromosome 6 at one or more of a nucleotide plus or minus 10, 20, 30, 40 50, 60, 80, 100, 150 nucleotides (corresponding to the breakpoint of a HLA-A-ROS1 fusion), or a fragment thereof, or a sequence substantially identical thereto. In one embodiment, the nucleic acid molecule is complementary to at least a portion of a nucleotide sequence disclosed herein, e.g., is capable of hybridizing under a stringency condition described herein to SEQ ID NO:37 and/or SEQ ID NO: 11 or a fragment thereof. In yet other embodiment, the nucleic acid molecule hybridizes to a nucleotide sequence that is complementary to at least a portion of a nucleotide sequence disclosed herein, e.g., is capable of hybridizing under a stringency condition described herein to a nucleotide sequence complementary to SEQ ID NO:37 or SEQ ID NO:11 or a fragment thereof.

In another embodiment, the HLA-A-ROS1 fusion nucleic acid comprises at least 6, 12, 15, 20, 25, 50, 75, 100 or more nucleotides from exon 7 of HLA-A (e.g., from the nucleotide sequence of HLA-A preceding the fusion junction with ROS1, e.g., of the HLA-A sequence shown in SEQ ID NO:37, and at least 6, 12, 15, 20, 25, 50, 75, 100 or more nucleotides from exon 34 of ROS1 (e.g., from the nucleotide sequence of ROS1 following the fusion junction with HLA-A, e.g., of the ROS1 sequence shown in SEQ ID NO:11.

In other embodiments, the nucleic acid molecule includes a nucleotide sequence encoding a HLA-A-ROS1 fusion polypeptide that includes a fragment of a HLA-A gene and a fragment of a ROS1 gene. In one embodiment, the nucleotide sequence encodes a HLA-A-ROS1 fusion polypeptide that includes e.g., a tyrosine kinase domain or a functional fragment thereof. In yet other embodiments, the nucleic acid molecule includes a nucleotide sequence encoding the amino acid sequence (e.g., a fragment of the amino acid sequence) shown in SEQ ID NO:38 and a nucleotide sequence encoding the amino acid sequence (e.g., a fragment of the amino acid sequence) shown in SEQ ID NO:12, or a fragment of the fusion, or a sequence substantially identical thereto. In one embodiment, the encoded HLA-A-ROS1 fusion polypeptide includes a tyrosine kinase domain or a functional fragment thereof.

In one embodiment, the HLA-A-ROS1 fusion includes the full sequence or a fragment of the nucleotide sequence shown in SEQ ID NO:35 and SEQ ID NO:82, or a nucleotide sequence substantially identical thereto. In another embodiment, the HLA-A-ROS1 fusion polypeptide includes the full sequence or a fragment of the amino acid sequence shown in SEQ ID NO:36 and SEQ ID NO:83, or an amino acid sequence substantially identical thereto.

In a related aspect, the invention features nucleic acid constructs that include the HLA-A-ROS1 nucleic acid molecules described herein. In certain embodiments, the nucleic acid molecules are operatively linked to a native or a heterologous regulatory sequence. Also included are vectors and host cells that include the HLA-A-ROS1 nucleic acid molecules described herein, e.g., vectors and host cells suitable for producing the nucleic acid molecules and polypeptides described herein.

In a related aspect, methods of producing the nucleic acid molecules and polypeptides described herein are also described.

In another aspect, the invention features nucleic acid molecules that reduce or inhibit the expression of a nucleic acid molecule that encodes a HLA-A-ROS1 fusion described herein. Examples of such nucleic acid molecules include, for example, antisense molecules, ribozymes, RNAi, triple helix molecules that hybridize to a nucleic acid encoding HLA-A-ROS1, or a transcription regulatory region of HLA-A-ROS1, and blocks or reduces mRNA expression of HLA-A-ROS1.

### Nucleic Acid Detection and Capturing Reagents

The invention also features a nucleic acid molecule, e.g., nucleic acid fragment, suitable as probe, primer, bait or library member that includes, flanks, hybridizes to, which are useful for identifying, or are otherwise based on, the HLA-A-ROS1 fusions described herein. In certain embodiments, the probe, primer or bait molecule is an oligonucleotide that allows capture, detection or isolation of a HLA-A-ROS1 fusion nucleic acid molecule described herein. The oligonucleotide can comprise a nucleotide sequence substantially complementary to a fragment of the HLA-A-ROS1 fusion nucleic acid molecules described herein. The sequence identity between the nucleic acid fragment, e.g., the oligonucleotide, and the target HLA-A-ROS1 sequence need not be exact, so long as the sequences are sufficiently complementary to allow the capture, detection or isolation of the target sequence. In one embodiment, the nucleic acid fragment is a probe or primer that includes an oligonucleotide between about 5 and 25, e.g., between 10 and 20, or 10 and 15 nucleotides in length. In other embodiments, the nucleic acid fragment is a bait that includes an oligonucleotide between about 100 to 300 nucleotides, 152 and 230 nucleotides, or 150 and 200 nucleotides, in length.

In one embodiment, the nucleic acid fragment can be used to identify or capture, e.g., by hybridization, a HLA-A-ROS1 fusion. For example, the nucleic acid fragment can be a probe, a primer, or a bait, for use in identifying or capturing, e.g., by hybridization, a HLA-A-ROS1 fusion described herein. In one embodiment, the nucleic acid fragment can be useful for identifying or capturing a HLA-A-ROS1 breakpoint, e.g., the nucleotide sequence of: chromosome 6 at nucleotide plus or minus 10, 20, 30, 40, 50, 60, 80, 100, 150 nucleotides and chromosome 6 at nucleotide plus or minus 10, 20, 30, 40 50, 60, 80, 100, 150 nucleotides.

In one embodiment, the nucleic acid fragment hybridizes to a nucleotide sequence within a chromosomal rearrangement that creates an in-frame fusion of intron 7 of HLA-A with intron 33 of ROS1. In one embodiment, the nucleic acid fragment hybridizes to a nucleotide sequence in the region In other embodiments, the nucleic acid molecules includes a nucleotide sequence in the region of a nucleotide sequence of chromosome 6 coupled to (e.g., juxtaposed to) nucleotides in the region of a nucleotide sequence of chromosome 6. In one embodiment, the nucleic acid fragment hybridizes to a nucleotide sequence that includes a breakpoint, e.g., the nucleotide sequence of: chromosome 6 at nucleotide plus or minus 10, 20, 30, 40 50, 60, 80, 100, 150 or more nucleotides and chromosome 6 at nucleotide plus or minus 10, 20, 30, 40 50, 60, 80, 100, 150 or more nucleotides. For example, the nucleic acid fragment can hybridize to a nucleotide sequence that includes the fusion junction between the HLA-A gene and the ROS1 gene, e.g., a nucleotide sequence that includes a portion of a nucleotide sequence within intron 7 of a HLA-A gene and intron 33 of a ROS1 gene.

In another embodiment, the nucleic acid fragment hybridizes to a nucleotide sequence that comprises at least 6, 12, 15, 20, 25, 50, 75, 100, 150 or more nucleotides from exon 7 of HLA-A (e.g., from the nucleotide sequence of HLA-A preceding the fusion junction with ROS1, e.g., of the HLA-A sequence shown in SEQ ID NO:37, and at least 6, 12, 15, 20, 25, 50, 75, 100, 150 or more nucleotides from exon 34 of ROS1 (e.g., from the nucleotide sequence of ROS1 following the fusion junction with HLA-A, e.g., of the ROS1 sequence shown in SEQ ID NO:11.

The probes or primers described herein can be used, for example, for FISH detection or PCR amplification. In one exemplary embodiment where detection is based on PCR, amplification of the HLA-A-ROS1 fusion junction fusion junction can be performed using a primer or a primer pair, e.g., for amplifying a sequence flanking the fusion junctions described herein, e.g., the mutations or the junction of a chromosomal rearrangement described herein, e.g., HLA-A-ROS1.

In one embodiment, a pair of isolated oligonucleotide primers can amplify a region containing or adjacent to a position in the HLA-A-ROS1 fusion. For example, forward primers can be designed to hybridize to a nucleotide sequence within HLA-A genomic or mRNA sequence (e.g., a nucleotide sequence within exon 7 of HLA-A of SEQ ID NO:37, and the reverse primers can be designed to hybridize to a nucleotide sequence of ROS1 (e.g., a nucleotide sequence within exon 34 of ROS1, of SEQ ID NO:11.

In another embodiment, the nucleic acid fragments can be used to identify, e.g., by hybridization, a HLA-A-ROS1 fusion. In one embodiment, the nucleic acid fragment hybridizes to a nucleotide sequence that includes a fusion junction between the HLA-A transcript and the ROS1 transcript.

In other embodiments, the nucleic acid fragment includes a bait that comprises a nucleotide sequence that hybridizes to a HLA-A-ROS1 fusion nucleic acid molecule described herein, and thereby allows the capture or isolation said nucleic acid molecule. In one embodiment, a bait is suitable for solution phase hybridization. In other embodiments, a bait includes a binding entity, e.g., an affinity tag, that allows capture and separation, e.g., by binding to a binding entity, of a hybrid formed by a bait and a nucleic acid hybridized to the bait.

In other embodiments, the nucleic acid fragment includes a library member comprising a HLA-A-ROS1 nucleic acid molecule described herein. In one embodiment, the library member includes a rearrangement that results in a HLA-A-ROS1 fusion described herein.

The nucleic acid fragment can be detectably labeled with, e.g., a radiolabel, a fluorescent label, a bioluminescent label, a chemiluminescent label, an enzyme label, a binding pair label, or can include an affinity tag; a tag, or identifier (e.g., an adaptor, barcode or other sequence identifier).

### HLA-A-ROS1 Fusion Polypeptides

In another embodiment, the HLA-A-ROS1 fusion comprises an amino acid sequence (e.g., a fragment of the amino acid sequence) shown in SEQ ID NO:38 and an amino acid sequence (e.g., a fragment of the amino acid sequence) shown in SEQ ID NO:12, or a fragment of the fusion. In one embodiment, the HLA-A-ROS1 fusion comprises an amino acid sequence substantially identical to the amino acid sequence (e.g., a fragment of the amino acid sequence) shown in SEQ ID NO:38 and the amino acid sequence (e.g., a fragment of the amino acid sequence) shown in SEQ ID NO:12, or a fragment thereof. In one embodiment, the HLA-A-ROS1 fusion comprises an amino acid sequence at least 70%, at least 75%, at least 80%, at least 85%, at least 90%, at least 95%, at least 99%, at least 99.5 or greater, identical to the amino acid sequence (e.g., a fragment of the amino acid sequence) shown in SEQ ID NO:38 and the amino acid sequence (e.g., a fragment of the amino acid sequence) shown in SEQ ID NO:12. In one embodiment, the HLA-A-ROS1 fusion comprises a sequence containing at least 10, 20, 50, 100, 500, 600, 700, 800, 900, 1000, or more amino acids of the amino acid sequence shown in SEQ ID NO:38 and SEQ ID NO: 12. In one embodiment, the HLA-A-ROS1 fusion comprises an amino acid sequence containing at least 5, 10, 20, 50, 100, 500, 600, 700, 800, 900, 1000, or more contiguous amino acids of the amino acid sequence shown in SEQ ID NO:38 and at least 5, 10, 20, 50, 100, 500, 600, 700, 800, 900, 1000, or more contiguous amino acids of the amino acid sequence shown in SEQ ID NO:12. In one embodiment, the 5′ HLA-A-3′ ROS1 fusion polypeptide includes a receptor tyrosine kinase domain or a functional fragment thereof. In an embodiment, the 5′HLA-A-3′ROS1 fusion polypeptide comprises sufficient ROS1 and sufficient HLA-A sequence such that it has kinase activity. e.g., has elevated activity. In any event, the fusion causes activation in the cells harboring it of oncogenic signaling pathways.

In another aspect, the invention features a HLA-A-ROS1 fusion polypeptide (e.g., a purified HLA-A-ROS1 fusion polypeptide), a biologically active or antigenic fragment thereof, as well as reagents (e.g., antibody molecules that bind to a HLA-A-ROS1 fusion polypeptide), methods for modulating a HLA-A-ROS1 polypeptide activity and detection of a HLA-A-ROS1 polypeptide.

In one embodiment, the HLA-A-ROS1 fusion polypeptide has at least one biological activity. In one embodiment, at least one biological activity of the HLA-A-ROS1 fusion polypeptide is reduced or inhibited by an anti-cancer drug, e.g., a HLA-A inhibitor, a ROS1 inhibitor. In one embodiment, at least one biological activity of the HLA-A-ROS1 fusion polypeptide is reduced or inhibited by a ROS1 inhibitor. In one embodiment, at least one biological activity of the HLA-A-ROS1 fusion polypeptide is reduced or inhibited by a HLA-A inhibitor. In one embodiment, at least one biological activity of the HLA-A-ROS1 fusion polypeptide is reduced or inhibited by a ROS1 inhibitor, e.g., Ganetespib; Crizotinib; TAE684; a dual ALK and ROS1 inhibitor.

In yet other embodiments, the HLA-A-ROS1 fusion polypeptide is encoded by a nucleic acid molecule described herein. In one embodiment, the HLA-A-ROS1 fusion polypeptide is encoded by an in-frame fusion of intron 7 of HLA-A with intron 33 of ROS1 (e.g., a sequence on chromosome 12 and a sequence on chromosome 6. In another embodiment, the HLA-A-ROS1 fusion polypeptide includes an amino acid sequence encoded by a nucleotide sequence comprising a fusion junction between the HLA-A transcript and the ROS1 transcript.

In certain embodiments, the HLA-A-ROS1 fusion polypeptide comprises one or more of encoded exons 1-7 from HLA-A and one or more of encoded exons 34-43 of ROS1. In certain embodiments, the HLA-A-ROS1 fusion polypeptide comprises at least 1, 2, 3, 4, 5, 6, 7 or more encoded exons of HLA-A and at least at least 1, 2, 3, 4, 5, 6, 7, 8, 9, 10 or more encoded exons of ROS1. In certain embodiments, the HLA-A-ROS1 fusion polypeptide comprises a fusion of encoded exon 7 from HLA-A and encoded exon 34 from ROS1 (or a fragment thereof). In other embodiments, the fusion comprises at least 1, 2, 3, 4, 5, 6, 7 or more encoded exons of HLA-A; and at least 1, 2, 3, 4, 5, 6, 7, 8, 9, 10 or more encoded exons of ROS1. In certain embodiments, the HLA-A-ROS1 fusion polypeptide comprises encoded exons 1-7 from HLA-A and exons 34-43 of ROS1. In certain embodiments, the 5′ HLA-A-3′ ROS1 fusion polypeptide comprises a fusion junction of the sequence of exon 7 from HLA-A and the sequence of exon 34 from ROS1.

In certain embodiments, the HLA-A-ROS1 fusion comprises the amino acid sequence corresponding to exon 7 or a fragment thereof from HLA-A, and the amino acid sequence corresponding to exon 34 or a fragment thereof from ROS1 (e.g., as shown in SEQ ID NO:38 and SEQ ID NO:12. In one embodiment, the HLA-A-ROS1 fusion comprises at least 5, 10, 15, 20 or more amino acids from exon 7 of HLA-A (e.g., from the amino acid sequence of HLA-A preceding the fusion junction with ROS1, e.g., of the HLA-A sequence shown in EQ ID NO:38, and at least 5, 10, 15, 20 or more amino acids from exon 34 of ROS1 (e.g., from the amino acid sequence of ROS1 following the fusion junction with HLA-A, e.g., of the ROS1 sequence shown in SEQ ID NO: 12.

In one embodiment, the HLA-A-ROS1 fusion polypeptide includes a tyrosine kinase domain or a functional fragment thereof. In a related aspect, the invention features HLA-A-ROS1 fusion polypeptide or fragments operatively linked to heterologous polypeptides to form fusion proteins.

In another embodiment, the HLA-A-ROS1 fusion polypeptide or fragment is a peptide, e.g., an immunogenic peptide or protein containing a fusion junction described herein. Such immunogenic peptides or proteins can be used to raise antibodies specific to the fusion protein. In other embodiments, such immunogenic peptides or proteins can be used for vaccine preparation. The vaccine preparation can include other components, e.g., an adjuvant.

In another aspect, the invention features antibody molecules that bind to a HLA-A-ROS1 fusion polypeptide or fragment described herein. In embodiments, the antibody can distinguish wild type ROS1 (or HLA-A) from HLA-A-ROS1.

### Detection Reagents and Detection of Mutations

In another aspect, the invention features a detection reagent, e.g., a purified or an isolated preparation thereof. Detection reagents can distinguish a nucleic acid, or protein sequence, having a breakpoint, e.g., a HLA-A-ROS1 breakpoint; from a reference sequence. In one embodiment, the detection reagent detects (e.g., specifically detects) a HLA-A-ROS1 fusion nucleic acid or a polypeptide, e.g., distinguishes a wild type ROS1 or another ROS1 fusion (or HLA-A) from a HLA-A-ROS1 nucleic acid (e.g., as described herein in SEQ ID NO:37 and SEQ ID NO: 12); or a HLA-A-ROS1 polypeptide (e.g., as described herein in SEQ ID NO:38 and SEQ ID NO:12).

Detection reagents, e.g., nucleic acid-based detection reagents, can be used to identify mutations in a target nucleic acid, e.g., DNA, e.g., genomic DNA or cDNA, or RNA, e.g., in a sample, e.g., a sample of nucleic acid derived from a neoplastic or tumor cell, e.g., a melanocytic neoplasm, melanoma or metastatic cell. Detection reagents, e.g., antibody-based detection reagents, can be used to identify the foregoing fusion mutations in a target protein, e.g., in a sample, e.g., a sample of protein derived from, or produced by, a neoplastic or tumor cell, e.g., a melanocytic neoplasm, melanoma or metastatic cell.

### Method of Treatment

In a related aspect, a method of inhibiting, reducing, or treating a hyperproliferative disorder, e.g., a neoplasm (including benign, pre-malignant or malignant (e.g., a cancer), in a subject is provided. The method includes administering to the subject a preselected therapeutic agent, e.g., an anti-cancer agent (e.g., a kinase inhibitor), as a single agent, or in combination, in an amount sufficient to reduce, inhibit or treat the activity or expression of HLA-A-ROS1 (e.g., a HLA-A-ROS1 fusion described herein), thereby inhibiting, reducing, or treating the hyperproliferative disorder in the subject.

In one embodiment, the subject treated has a HLA-A-ROS1 fusion; e.g., the subject has a tumor or cancer harboring a HLA-A-ROS1 fusion. In other embodiments, the subject has been previously identified as having a HLA-A-ROS1 fusion. In yet other embodiments, the subject has been previously identified as being likely or unlikely to respond to treatment with a protein kinase inhibitor, e.g., a subject that has previously participated in a clinical trial. In other embodiments, the subject has been previously identified as being likely or unlikely to respond to treatment with a protein kinase inhibitor, based on the presence of the HLA-A-ROS1 fusion. In one embodiment, the subject is a mammal, e.g., a human. In one embodiment, the subject has, or at risk of having a cancer at any stage of disease. In other embodiments, the subject is a patient, e.g., a cancer patient.

In certain embodiments, the neoplasm or neoplastic cell is a benign, pre-malignant, malignant (cancer) or metastasis. In certain embodiments, the cancer is a solid tumor, a soft tissue tumor, or a metastatic lesion.

In one embodiment, the neoplasm or neoplastic cell is a benign, pre-malignant, malignant (cancer) or metastasis. In certain embodiments, the cancer is a solid tumor, a soft tissue tumor, or a metastatic lesion. In one embodiment, the cancer is a carcinoma. In one embodiment, the cancer is chosen from a lung cancer, a pancreatic cancer, melanoma, a colorectal cancer, an esophageal-gastric cancer, a thyroid cancer, or an adenocarcinoma. In one embodiment, the cancer is a lung adenocarcinoma. In other embodiment, the lung cancer is chosen from one or more of the following: non-small cell lung cancer (NSCLC), small cell lung cancer SCLC), squamous cell carcinoma SCC), adenocarcinoma of the lung, bronchogenic carcinoma, or a combination thereof. In one embodiment, the lung cancer is NSCLC or SCC.

In one embodiment, the anti-cancer agent is a ROS1 inhibitor. In one embodiment, the anti-cancer agent is a HLA-A inhibitor. In one embodiment, the anti-cancer agent is a kinase inhibitor. In one embodiment, the anti-cancer agent is a ROS1 inhibitor, e.g., Ganetespib; Crizotinib; TAE684; a dual ALK and ROS1 inhibitor.

### ERC1-ROS1 Fusions

In one embodiment, a fusion includes an in-frame fusion of an exon of ELKS/RAB6-interacting/CAST family member 1 (ERC1, e.g., one more exons of ERC1 (e.g., one or more of exons 1-111 of ERC1 or a fragment thereof, and an exon of C-Ros oncogene 1 (ROS1, e.g., one or more exons of a ROS1 (e.g., one or more of exons 36-43 of ROS1 or a fragment thereof. For example, the ERC1-ROS1 fusion can include an in-frame fusion within an intron of ERC1 (e.g., intron 11 or a fragment thereof, with an intron of ROS1 (e.g., intron 35 or a fragment thereof. In one embodiment, the fusion of the ERC1-ROS1 fusion comprises the nucleotide sequence of: chromosome 12 at one or more of a nucleotide (plus or minus 10, 20, 30, 50, 60, 70, 80, 100 or more nucleotides) and chromosome 6 at one or more of a nucleotide (plus or minus 10, 20, 30, 50, 60, 70, 80, 100 or more nucleotides). In one embodiment, the ERC1-ROS1 fusion is a translocation, e.g., a translocation of a portion of chromosome 6 and a portion of chromosome 6.

In certain embodiments, the ERC1-ROS1 fusion is in a 5′-ERC1 to 3′-ROS1 configuration (also referred to herein as “5′-ERC1-ROS1-3′).” The term “fusion” or “fusion molecule” anywhere in this specification can refer to a polypeptide or a nucleic acid fusion, depending on the context. It may include a full-length sequence of a fusion or a fragment thereof, e.g., a fusion junction (e.g., a fragment including a portion of ERC1 and a portion of ROS1, e.g., a portion of the ERC1-ROS1 fusion described herein). In one embodiment, the ERC1-ROS1 fusion polypeptide includes a fragment of the amino acid sequence shown in SEQ ID NO:42 and a fragment of the amino acid sequence shown in SEQ ID NO: 12, or an amino acid sequence substantially identical thereto. In another embodiment, the ERC1-ROS1 fusion nucleic acid includes a fragment of the nucleotide sequence shown in SEQ ID NO:41 and a fragment of the nucleotide sequence shown in SEQ ID NO: 11, or a nucleotide sequence substantially identical thereto. In one embodiment, the ERC1-ROS fusion polypeptide comprises sufficient ERC1 and sufficient ROS1 sequence such that the 5′ ERC1-3′ ROS1 fusion has kinase activity, e.g., has elevated activity, e.g., tyrosine kinase activity. In any event, the fusion causes activation in the cells harboring it of oncogenic signaling pathways.

In certain embodiments, the ERC1-ROS1 fusion comprises one or more (or all of) exons 1-11 from ERC1 and one or more (or all of) exons 36-43 of ROS1 (e.g., one or more of the exons shown in SEQ ID NO:41 and SEQ ID NO: 11. In another embodiment, the ERC1-ROS1 fusion comprises one or more (or all of) exons 1-11 of ERC1 and one or more (or all of) exons 36-43 of ROS1. In certain embodiments, the ERC1-ROS1 fusion comprises at least 1, 2, 3, 4, 5, 6, 7, 8, 9, 10, 11 or more exons (or encoded exons) from ERC1 and at least 1, 2, 3, 4, 5, 6, 7, 8 or more exons (or encoded exons) from ROS1 (e.g., from the ERC1 and ROS1 sequences shown in SEQ ID NO:41 and SEQ ID NO:42 and SEQ ID NO:11 and SEQ ID NO:12.

In certain embodiments, the ERC1-ROS1 fusion comprises exons 1-11 or a fragment thereof from ERC1, and exons 36-43 or a fragment thereof from ROS1 (e.g., as shown in SEQ ID NO:41 and SEQ ID NO: 11. In one embodiment, the ERC1-ROS1 fusion comprises at least 5, 10, 15, 20, 30, 40, 50 or more amino acids from exons 1-11 of ERC1 (e.g., from the amino acid sequence of ERC1 as shown in SEQ ID NO:42 (e.g., from the amino acid sequence of ERC1 preceding the fusion junction with ROS1, and at least 5, 10, 15, 20, 30, 40, 50 or more amino acids from exons 36-43 of ROS1 (e.g., from the amino acid sequence of ROS1 as shown in SEQ ID NO:12. In another embodiment, the ERC1-ROS1 fusion comprises at least 6, 12, 15, 20, 25, 50, 75, 100 or more nucleotides from exons 1-11of ERC1 (e.g., from the nucleotide sequence of ERC1 as shown in SEQ ID NO:41 (e.g., from the nucleotide sequence of ERC1 preceding the fusion junction with ROS1; and at least 6, 12, 15, 20, 25, 50, 75, 100 or more nucleotides from exons 36-43 of ROS1 (e.g., from the nucleotide sequence of ROS1 as shown in SEQ ID NO:11.

### ERC1-ROS1 Nucleic Acid Molecules

In one aspect, the invention features a nucleic acid molecule (e.g., an isolated or purified) nucleic acid molecule that includes a fragment of an ERC1 gene and a fragment of a ROS1 gene. In one embodiment, the nucleotide sequence encodes an ERC1-ROS1 fusion polypeptide that includes a tyrosine kinase domain or a functional fragment thereof. In another embodiment, the nucleotide sequence encodes a fragment of the ROS1 polypeptide including the amino acid sequence of SEQ ID NO: 12 or a fragment thereof, or a sequence substantially identical thereto. In other embodiments, the nucleic acid molecule includes a fragment of the ERC1 gene encoding the amino acid sequence of SEQ ID NO:42 or a fragment thereof, or a sequence substantially identical thereto. In yet other embodiments, the nucleic acid molecule includes a nucleotide sequence encoding the amino acid sequence shown in SEQ ID NO:42, or a fragment thereof, and the amino acid sequence shown in SEQ ID NO: 12 or a fragment thereof, or a sequence substantially identical thereto.

In one embodiment, the nucleic acid molecule includes a fusion, e.g., an in-frame fusion, between an intron of ERC1 (e.g., intron 11, or a fragment thereof), and an intron of ROS1 (e.g., intron 35, or a fragment thereof). The ERC1-ROS1 fusion can comprise a fusion of the nucleotide sequence of: chromosome 12 at one or more of a nucleotide (plus or minus 10, 20, 30, 50, 60, 70, 80, 100 nucleotides) and chromosome 6 at one or more of a nucleotide (plus or minus 10, 20, 30, 50, 60, 70, 80, 100 nucleotides), or a fragment thereof. In one embodiment, the ERC1-ROS1 fusion comprises a fusion of the nucleotide sequence of: chromosome 6 at one or more of a nucleotide (plus or minus 10, 20, 30, 50, 60, 70, 80, 100 nucleotides) and chromosome 6 at one or more of a nucleotide (plus or minus 10, 20, 30, 50, 60, 70, 80, 100 nucleotides), or a fragment thereof.

In another embodiment, the ERC1-ROS1 fusion comprises a nucleotide sequence (e.g., a fragment of a nucleotide sequence) shown in SEQ ID NO:41 and a nucleotide sequence (e.g., a fragment of a nucleotide sequence) shown in SEQ ID NO: 11, or a fragment of the fusion. In one embodiment, the ERC1-ROS1 fusion comprises a nucleotide sequence substantially identical to the nucleotide sequence (e.g., a fragment of a nucleotide sequence) shown in SEQ ID NO:41 and the nucleotide sequence (e.g., a fragment of a nucleotide sequence) shown SEQ ID NO: 11, or a fragment of the fusion. In one embodiment, the ERC1-ROS1 fusion comprises a nucleotide sequence at least 70%, at least 75%, at least 80%, at least 85%, at least 90%, at least 95%, at least 99%, at least 99.5 or greater, identical to the nucleotide sequence (e.g., a fragment of a nucleotide sequence) shown in SEQ ID NO:41 and to the nucleotide sequence (e.g., a fragment of a nucleotide sequence) shown in SEQ ID NO: 11. In one embodiment, the ERC1-ROS1 fusion comprises a nucleotide sequence containing at least 25, 50, 100, 150, 200, 500, 1000, 1500, 2000, 2500, 3000, or more nucleotides of the nucleotide sequence shown in SEQ ID NO:41 and a nucleotide sequence containing at least 25, 50, 100, 150, 200, 500, 1000, 1500, 2000, 2500, 3000, or more nucleotides of the nucleotide sequence shown in SEQ ID NO:11. In one embodiment, the ERC1-ROS1 fusion comprises a nucleotide sequence containing at least 25, 50, 100, 150, 200, 500, 1000, 1500, 2000, 2500, 3000, or more contiguous nucleotides of the nucleotide sequence shown in SEQ ID NO:41 and a nucleotide sequence containing at least 25, 50, 100, 150, 200, 500, 10(00), 1500, 2000, 2500, 3000, or more nucleotides of the nucleotide sequence shown in SEQ ID NO: 11.

In another embodiment, the nucleic acid molecule includes a fusion, e.g., an in-frame fusion, of at least 1, 2, 3, 4, 5, 6, 7, 8, 9, 10, 11 or more exons of ERC1 or a fragment thereof (e.g., one or more of exons 1-11 of ERC1 or a fragment thereof), and at least 1, 2, 3, 4, 5, 6, 7, 8 or more exons of ROS1 or a fragment thereof (e.g., one or more of exons 36-43 of ROS1 or a fragment thereof). In yet other embodiments, the nucleic acid molecule includes a fragment the nucleotide sequence shown in SEQ ID NO:41 and a fragment of the nucleotide sequence shown in SEQ ID NO:11 or a fragment of the fusion, or a sequence substantially identical thereto.

In one embodiment, the nucleic acid molecule is complementary to at least a portion of a nucleotide sequence disclosed herein, e.g., is capable of hybridizing under a stringency condition described herein to SEQ ID NO:41 and/or SEQ ID NO: 11, or a fragment thereof. In yet another embodiment, the nucleic acid molecule hybridizes to a nucleotide sequence that is complementary to at least a portion of a nucleotide sequence disclosed herein, e.g., is capable of hybridizing under a stringency condition to a nucleotide sequence complementary to SEQ ID NO:41 and/or SEQ ID NO: 11, or a fragment thereof. The nucleotide sequence of a cDNA encoding an exemplary 5′ ERC1-3′ ROS1 fusion is shown in at least exon 11 (e.g., exons 1-11 of SEQ ID NO:41 and at least exon 36 (e.g., exons 36-43 of SEQ ID NO: 11, and the predicted amino acid sequence is shown in the corresponding encoded exons of SEQ ID NO:42 and the corresponding encoded exons of SEQ ID NO: 12, respectively.

In an embodiment the ERC1-ROS1 nucleic acid molecule comprises sufficient ERC1 and sufficient ROS1 sequence such that the encoded 5′ ERC1-3′ ROS1 fusion has kinase activity, e.g., has elevated activity. For example, in this embodiment, ERC1 has no kinase activity before fusion and ROS1 is not constitutively activated. In certain embodiments, the 5′ ERC1-3′ ROS1 fusion comprises exons 1-11 from ERC1 and exons 36-43 from ROS1. In certain embodiments, the ERC1-ROS1 fusion comprises at least 1, 2, 3, 4, 5, 6, 7, 8, 9, 10, 11 or more exons from ERC1 and at least at least 1, 2, 3, 4, 5, 6, 7, 8 or more exons of ROS1. In certain embodiments, the ERC1-ROS1 fusion comprises a fusion of exon 11 from ERC1 and exon 36 from ROS1. In another embodiment, the ERC1-ROS1 fusion comprises 1, 2, 3, 4, 5, 6, 7, 8, 9, 10, 11 or more exons of ERC1; and at least 1, 2, 3, 4, 5, 6, 7, 8 or more exons of ROS1.

In one embodiment, the nucleic acid molecule includes a nucleotide sequence that has an in-frame fusion of intron 11 of ERC1 (e.g., NM_{—}178039 with intron 36 of ROS1 (e.g., NM_{—}002944. In another embodiment, the nucleic acid molecule includes a nucleotide sequence that includes a breakpoint. For example, the nucleic acid molecule includes a nucleotide sequence that includes the fusion junction between the ERC1 gene and the ROS1 gene, e.g., the breakpoint between intron 11 of ERC1 and intron 35 of ROS1. In other embodiments, the nucleic acid molecules includes a nucleotide sequence of one or more of nucleotide of chromosome 6 coupled to (e.g., directly or indirectly juxtaposed to) one or more of nucleotide of chromosome 6. In one embodiment, the nucleic acid molecule includes the nucleotide sequence of: chromosome 6 at one or more of a nucleotide plus or minus 10, 20, 30, 40 50, 60, 80, 100, 150 nucleotides and chromosome 6 at one or more of a nucleotide plus or minus 10, 20, 30, 40 50, 60, 80, 100, 150 nucleotides (corresponding to the breakpoint of a ERC1-ROS1 fusion), or a fragment thereof, or a sequence substantially identical thereto. In one embodiment, the nucleic acid molecule is complementary to at least a portion of a nucleotide sequence disclosed herein, e.g., is capable of hybridizing under a stringency condition described herein to SEQ ID NO:41 and/or SEQ ID NO:11 or a fragment thereof. In yet other embodiment, the nucleic acid molecule hybridizes to a nucleotide sequence that is complementary to at least a portion of a nucleotide sequence disclosed herein, e.g., is capable of hybridizing under a stringency condition described herein to a nucleotide sequence complementary to SEQ ID NO:41 or SEQ ID NO:11 or a fragment thereof.

In another embodiment, the ERC1-ROS1 fusion nucleic acid comprises at least 6, 12, 15, 20, 25, 50, 75, 100 or more nucleotides from exon 11 of ERC1 (e.g., from the nucleotide sequence of ERC1 preceding the fusion junction with ROS1, e.g., of the ERC1 sequence shown in SEQ ID NO:41, and at least 6, 12, 15, 20, 25, 50, 75, 100 or more nucleotides from exon 36 of ROS1 (e.g., from the nucleotide sequence of ROS1 following the fusion junction with ERC1, e.g., of the ROS1 sequence shown in SEQ ID NO:11.

In other embodiments, the nucleic acid molecule includes a nucleotide sequence encoding a ERC1-ROS1 fusion polypeptide that includes a fragment of a ERC1 gene and a fragment of a ROS gene. In one embodiment, the nucleotide sequence encodes an ERC1-ROS1 fusion polypeptide that includes e.g., a tyrosine kinase domain or a functional fragment thereof. In yet other embodiments, the nucleic acid molecule includes a nucleotide sequence encoding the amino acid sequence (e.g., a fragment of the amino acid sequence) shown in SEQ ID NO:42 and a nucleotide sequence encoding the amino acid sequence (e.g., a fragment of the amino acid sequence) shown in SEQ ID NO:12, or a fragment of the fusion, or a sequence substantially identical thereto. In one embodiment, the encoded ERC1-ROS1 fusion polypeptide includes a tyrosine kinase domain or a functional fragment thereof.

In one embodiment, the ERC1-ROS1 fusion includes the full sequence or a fragment of the nucleotide sequence shown in SEQ ID NO:39 and SEQ ID NO:88, or a nucleotide sequence substantially identical thereto. In another embodiment, the ERC1-ROS1 fusion polypeptide includes the full sequence or a fragment of the amino acid sequence shown in SEQ ID NO:40 and SEQ ID NO:89, or an amino acid sequence substantially identical thereto.

In a related aspect, the invention features nucleic acid constructs that include the ERC1-ROS1 nucleic acid molecules described herein. In certain embodiments, the nucleic acid molecules are operatively linked to a native or a heterologous regulatory sequence. Also included are vectors and host cells that include the ERC1-ROS1 nucleic acid molecules described herein, e.g., vectors and host cells suitable for producing the nucleic acid molecules and polypeptides described herein.

In a related aspect, methods of producing the nucleic acid molecules and polypeptides described herein are also described.

In another aspect, the invention features nucleic acid molecules that reduce or inhibit the expression of a nucleic acid molecule that encodes an ERC1-ROS1 fusion described herein. Examples of such nucleic acid molecules include, for example, antisense molecules, ribozymes, RNAi, triple helix molecules that hybridize to a nucleic acid encoding ERC1-ROS1, or a transcription regulatory region of ERC1-ROS1, and blocks or reduces mRNA expression of ERC1-ROS1.

### Nucleic Acid Detection and Capturing Reagents

The invention also features a nucleic acid molecule, e.g., nucleic acid fragment, suitable as probe, primer, bait or library member that includes, flanks, hybridizes to, which are useful for identifying, or are otherwise based on, the ERC1-ROS1 fusions described herein. In certain embodiments, the probe, primer or bait molecule is an oligonucleotide that allows capture, detection or isolation of a ERC1-ROS1 fusion nucleic acid molecule described herein. The oligonucleotide can comprise a nucleotide sequence substantially complementary to a fragment of the ERC1-ROS1 fusion nucleic acid molecules described herein. The sequence identity between the nucleic acid fragment. e.g., the oligonucleotide, and the target ERC1-ROS1 sequence need not be exact, so long as the sequences are sufficiently complementary to allow the capture, detection or isolation of the target sequence. In one embodiment, the nucleic acid fragment is a probe or primer that includes an oligonucleotide between about 5 and 25, e.g., between 10 and 20, or 10 and 15 nucleotides in length. In other embodiments, the nucleic acid fragment is a bait that includes an oligonucleotide between about 100 to 300 nucleotides, 156 and 230 nucleotides, or 150 and 200 nucleotides, in length.

In one embodiment, the nucleic acid fragment can be used to identify or capture, e.g., by hybridization, an ERC1-ROS1 fusion. For example, the nucleic acid fragment can be a probe, a primer, or a bait, for use in identifying or capturing, e.g., by hybridization, a ERC1-ROS1 fusion described herein. In one embodiment, the nucleic acid fragment can be useful for identifying or capturing a ERC1-ROS1 breakpoint, e.g., the nucleotide sequence of: chromosome 12 at nucleotide plus or minus 10, 20, 30, 40, 50, 60, 80, 100, 150 nucleotides and chromosome 6 at nucleotide plus or minus 10, 20, 30, 40 50, 60, 80, 100, 150 nucleotides.

In one embodiment, the nucleic acid fragment hybridizes to a nucleotide sequence within a chromosomal rearrangement that creates an in-frame fusion of intron 11 of ERC1 with intron 35 of ROS1. In one embodiment, the nucleic acid fragment hybridizes to a nucleotide sequence in the region In other embodiments, the nucleic acid molecules includes a nucleotide sequence in the region of a nucleotide sequence of chromosome 6 coupled to (e.g., juxtaposed to) nucleotides in the region of a nucleotide sequence of chromosome 6. In one embodiment, the nucleic acid fragment hybridizes to a nucleotide sequence that includes a breakpoint, e.g., the nucleotide sequence of: chromosome 12 at nucleotide plus or minus 10, 20, 30, 40, 50, 60, 80, 100, 150 or more nucleotides and chromosome 6 at nucleotide plus or minus 10, 20, 30, 40, 50, 60, 80, 100, 150 or more nucleotides. For example, the nucleic acid fragment can hybridize to a nucleotide sequence that includes the fusion junction between the ERC1 gene and the ROS1 gene, e.g., a nucleotide sequence that includes a portion of a nucleotide sequence within intron 11 of an ERC1 gene and intron 35 of a ROS1 gene.

In another embodiment, the nucleic acid fragment hybridizes to a nucleotide sequence that comprises at least 6, 12, 15, 20, 25, 50, 75, 100, 150 or more nucleotides from exon 11 of ERC1 (e.g., from the nucleotide sequence of ERC1 preceding the fusion junction with ROS1, e.g., of the ERC1 sequence shown in SEQ ID NO:41, and at least 6, 12, 15, 20, 25, 50, 75, 100, 150 or more nucleotides from exon 36 of ROS1 (e.g., from the nucleotide sequence of ROS1 following the fusion junction with ERC1, e.g., of the ROS1 sequence shown in SEQ ID NO: 11.

The probes or primers described herein can be used, for example, for FISH detection or PCR amplification. In one exemplary embodiment where detection is based on PCR, amplification of the ERC1-ROS1 fusion junction fusion junction can be performed using a primer or a primer pair, e.g., for amplifying a sequence flanking the fusion junctions described herein, e.g., the mutations or the junction of a chromosomal rearrangement described herein, e.g., ERC1-ROS1.

In one embodiment, a pair of isolated oligonucleotide primers can amplify a region containing or adjacent to a position in the ERC1-ROS1 fusion. For example, forward primers can be designed to hybridize to a nucleotide sequence within ERC1 genomic or mRNA sequence (e.g., a nucleotide sequence within exon 11 of ERC1 of SEQ ID NO:41, and the reverse primers can be designed to hybridize to a nucleotide sequence of ROS1 (e.g., a nucleotide sequence within exon 36 of ROS1, of SEQ ID NO: 11.

In another embodiment, the nucleic acid fragments can be used to identify, e.g., by hybridization, a ERC1-ROS1 fusion. In one embodiment, the nucleic acid fragment hybridizes to a nucleotide sequence that includes a fusion junction between the ERC1 transcript and the ROS1 transcript.

In other embodiments, the nucleic acid fragment includes a bait that comprises a nucleotide sequence that hybridizes to a ERC1-ROS1 fusion nucleic acid molecule described herein, and thereby allows the capture or isolation said nucleic acid molecule. In one embodiment, a bait is suitable for solution phase hybridization. In other embodiments, a bait includes a binding entity, e.g., an affinity tag, that allows capture and separation, e.g., by binding to a binding entity, of a hybrid formed by a bait and a nucleic acid hybridized to the bait.

In other embodiments, the nucleic acid fragment includes a library member comprising a ERC1-ROS1 nucleic acid molecule described herein. In one embodiment, the library member includes a rearrangement that results in the ERC1-ROS1 fusion described herein.

The nucleic acid fragment can be detectably labeled with, e.g., a radiolabel, a fluorescent label, a bioluminescent label, a chemiluminescent label, an enzyme label, a binding pair label, or can include an affinity tag; a tag, or identifier (e.g., an adaptor, barcode or other sequence identifier).

### ERC1-ROS1 Fusion Polypeptides

In another embodiment, the ERC1-ROS1 fusion comprises an amino acid sequence (e.g., a fragment of the amino acid sequence) shown in SEQ ID NO:42 and an amino acid sequence (e.g., a fragment of the amino acid sequence) shown in SEQ ID NO: 12, or a fragment of the fusion. In one embodiment, the ERC1-ROS1 fusion comprises an amino acid sequence substantially identical to the amino acid sequence (e.g., a fragment of the amino acid sequence) shown in SEQ ID NO:42 and the amino acid sequence (e.g., a fragment of the amino acid sequence) shown in SEQ ID NO:12, or a fragment thereof. In one embodiment, the ERC1-ROS1 fusion comprises an amino acid sequence at least 70%, at least 75%, at least 80%, at least 85%, at least 90%, at least 95%, at least 99%, at least 99.5 or greater, identical to the amino acid sequence (e.g., a fragment of the amino acid sequence) shown in SEQ ID NO:42 and the amino acid sequence (e.g., a fragment of the amino acid sequence) shown in SEQ ID NO:12. In one embodiment, the ERC11-ROS1 fusion comprises a sequence containing at least 10, 20, 50, 100, 500, 600, 700, 800, 900, 1000, or more amino acids of the amino acid sequence shown in SEQ ID NO:42 and SEQ ID NO: 12. In one embodiment, the ERC1-ROS1 fusion comprises an amino acid sequence containing at least 5, 10, 20, 50, 100, 500, 600, 700, 800, 900, 1000, or more contiguous amino acids of the amino acid sequence shown in SEQ ID NO:42 and at least 5, 10, 20, 50, 100, 500, 600, 700, 800, 900, 1000, or more contiguous amino acids of the amino acid sequence shown in SEQ ID NO:12. In one embodiment, the 5′ ERC1-3′ ROS1 fusion polypeptide includes a receptor tyrosine kinase domain or a functional fragment thereof. In an embodiment, the 5′ERC1-3′ROS1 fusion polypeptide comprises sufficient ROS1 and sufficient ERC1 sequence such that it has kinase activity. e.g., has elevated activity compared to a polypeptide that does not encode a kinase sequence or to a polypeptide wherein the kinase sequence is not constitutively “on.” In any event, the fusion causes activation in the cells harboring it of oncogenic signaling pathways.

In another aspect, the invention features an ERC1-ROS1 fusion polypeptide (e.g., a purified ERC1-ROS1 fusion polypeptide), a biologically active or antigenic fragment thereof, as well as reagents (e.g., antibody molecules that bind to a ERC1-ROS1 fusion polypeptide), methods for modulating a ERC1-ROS1 polypeptide activity and detection of a ERC1-ROS1 polypeptide.

In one embodiment, the ERC1-ROS1 fusion polypeptide has at least one biological activity. In one embodiment, at least one biological activity of the ERC1-ROS1 fusion polypeptide is reduced or inhibited by an anti-cancer drug, e.g., an ERC1 inhibitor, a ROS1 inhibitor. In one embodiment, at least one biological activity of the ERC1-ROS1 fusion polypeptide is reduced or inhibited by a ROS1 inhibitor. In one embodiment, at least one biological activity of the ERC1-ROS1 fusion polypeptide is reduced or inhibited by an ERC1 inhibitor. In one embodiment, at least one biological activity of the ERC1-ROS1 fusion polypeptide is reduced or inhibited by a ROS1 inhibitor, e.g., Ganetespib; Crizotinib; TAE684; a dual ALK and ROS1 inhibitor.

In yet other embodiments, the ERC1-ROS1 fusion polypeptide is encoded by a nucleic acid molecule described herein. In one embodiment, the ERC1-ROS1 fusion polypeptide is encoded by an in-frame fusion of intron 11 of ERC1 with intron 35 of ROS1 (e.g., a sequence on chromosome 12 and a sequence on chromosome 6. In another embodiment, the ERC1-ROS1 fusion polypeptide includes an amino acid sequence encoded by a nucleotide sequence comprising a fusion junction between the ERC1 transcript and the ROS1 transcript.

In certain embodiments, the ERC1-ROS1 fusion polypeptide comprises one or more of encoded exons 1-11 from ERC1 and one or more of encoded exons 36-43 of ROS1. In certain embodiments, the ERC1-ROS1 fusion polypeptide comprises at least 1, 2, 3, 4, 5, 6, 7, 8, 9, 10, 1 or more encoded exons of ERC1 and at least at least 1, 2, 3, 4, 5, 6, 7, 8 or more encoded exons of ROS1. In certain embodiments, the ERC1-ROS1 fusion polypeptide comprises a fusion of encoded exon 1 from ERC1 and encoded exon 36 from ROS1 (or a fragment thereof). In other embodiments, the fusion comprises at least 1, 2, 3, 4, 5, 6, 7, 8, 9, 10, 11 or more encoded exons of ERC1; and at least 1, 2, 3, 4, 5, 6, 7, 8 or more encoded exons of ROS1. In certain embodiments, the ERC1-ROS1 fusion polypeptide comprises encoded exons 1-11 from ERC1 and exons 36-43 of ROS1. In certain embodiments, the 5′ ERC1-3′ ROS1 fusion polypeptide comprises a fusion junction of the sequence of exon 11 from ERC1 and the sequence of exon 36 from ROS1.

In certain embodiments, the ERC1-ROS1 fusion comprises the amino acid sequence corresponding to exon 11 or a fragment thereof from ERC1 and the amino acid sequence corresponding to exon 36 or a fragment thereof from ROS1 (e.g., as shown in SEQ ID NO:42 and SEQ ID NO:12. In one embodiment, the ERC1-ROS1 fusion comprises at least 5, 10, 15, 20 or more amino acids from exon 11 of ERC1 (e.g., from the amino acid sequence of ERC1 preceding the fusion junction with ROS1, e.g., of the ERC1 sequence shown in EQ ID NO:42, and at least 5, 10, 15, 20 or more amino acids from exon 36 of ROS1 (e.g., from the amino acid sequence of ROS1 following the fusion junction with ERC1, e.g., of the ROS1 sequence shown in SEQ ID NO:12.

In one embodiment, the ERC1-ROS1 fusion polypeptide includes a tyrosine kinase domain or a functional fragment thereof. In a related aspect, the invention features ERC1-ROS1 fusion polypeptide or fragments operatively linked to heterologous polypeptides to form fusion proteins.

In another embodiment, the ERC1-ROS1 fusion polypeptide or fragment is a peptide, e.g., an immunogenic peptide or protein containing a fusion junction described herein. Such immunogenic peptides or proteins can be used to raise antibodies specific to the fusion protein. In other embodiments, such immunogenic peptides or proteins can be used for vaccine preparation. The vaccine preparation can include other components, e.g., an adjuvant.

In another aspect, the invention features antibody molecules that bind to a ERC1-ROS1 fusion polypeptide or fragment described herein. In embodiments, the antibody can distinguish wild type ROS1 or ERC1 from ERC1-ROS1.

### Detection Reagents and Detection of Mutations

In another aspect, the invention features a detection reagent, e.g., a purified or an isolated preparation thereof. Detection reagents can distinguish a nucleic acid, or protein sequence, having a breakpoint, e.g., an ERC1-ROS1 breakpoint; from a reference sequence. In one embodiment, the detection reagent detects (e.g., specifically detects) a ERC1-ROS1 fusion nucleic acid or a polypeptide, e.g., distinguishes a wild type ROS1 or another ROS1 fusion (or ERC1) from a ERC1-ROS1 nucleic acid (e.g., as described herein in SEQ ID NO:41 and SEQ ID NO:12); or a ERC1-ROS1 polypeptide (e.g., as described herein in SEQ ID NO:42 and SEQ ID NO:12).

Detection reagents, e.g., nucleic acid-based detection reagents, can be used to identify mutations in a target nucleic acid, e.g., DNA, e.g., genomic DNA or cDNA, or RNA. e.g., in a sample, e.g., a sample of nucleic acid derived from a neoplastic or tumor cell, e.g., a melanocytic neoplasm, melanoma or metastatic cell. Detection reagents, e.g., antibody-based detection reagents, can be used to identify mutations in a target protein, e.g., in a sample, e.g., a sample of protein derived from, or produced by, a neoplastic or tumor cell, e.g., a melanocytic neoplasm, melanoma or metastatic cell.

### Method of Treatment

In a related aspect, a method of inhibiting, reducing, or treating a hyperproliferative disorder, e.g., a neoplasm (including benign, pre-malignant or malignant (e.g., a cancer), in a subject is provided. The method includes administering to the subject a preselected therapeutic agent, e.g., an anti-cancer agent (e.g., a kinase inhibitor), as a single agent, or in combination, in an amount sufficient to reduce, inhibit or treat the activity or expression of ERC1-ROS1 (e.g., an ERC1-ROS1 fusion described herein), thereby inhibiting, reducing, or treating the hyperproliferative disorder in the subject.

In one embodiment, the subject treated has an ERC1-ROS1 fusion; e.g., the subject has a tumor or cancer harboring a ERC1-ROS1 fusion. In other embodiments, the subject has been previously identified as having an ERC1-ROS1 fusion. In yet other embodiments, the subject has been previously identified as being likely or unlikely to respond to treatment with a protein kinase inhibitor, e.g., a subject that has previously participated in a clinical trial. In other embodiments, the subject has been previously identified as being likely or unlikely to respond to treatment with a protein kinase inhibitor, based on the presence of the ERC1-ROS1 fusion. In one embodiment, the subject is a mammal, e.g., a human. In one embodiment, the subject has, or at risk of having a cancer at any stage of disease. In other embodiments, the subject is a patient, e.g., a cancer patient.

In certain embodiments, the neoplasm or neoplastic cell is a benign, pre-malignant, malignant (cancer) or metastasis. In certain embodiments, the cancer is a solid tumor, a soft tissue tumor, or a metastatic lesion.

In one embodiment, the neoplasm or neoplastic cell is a benign, pre-malignant, malignant (cancer) or metastasis. In certain embodiments, the cancer is a solid tumor, a soft tissue tumor, or a metastatic lesion. In one embodiment, the cancer is a carcinoma. In one embodiment, the cancer is chosen from a lung cancer, a pancreatic cancer, melanoma, a colorectal cancer, an esophageal-gastric cancer, a thyroid cancer, or an adenocarcinoma. In one embodiment, the cancer is a lung adenocarcinoma. In other embodiment, the lung cancer is chosen from one or more of the following: non-small cell lung cancer (NSCLC), small cell lung cancer SCLC), squamous cell carcinoma SCC), adenocarcinoma of the lung, bronchogenic carcinoma, or a combination thereof. In one embodiment, the lung cancer is NSCLC or SCC.

In one embodiment, the anti-cancer agent is a ROS1 inhibitor. In one embodiment, the anti-cancer agent is an ERC1 inhibitor. In one embodiment, the anti-cancer agent is a kinase inhibitor. In one embodiment, the anti-cancer agent is a ROS1 inhibitor, e.g., Ganetespib; Crizotinib; TAE684; a dual ALK and ROS1 inhibitor.

### KIAA1598-ROS1

In one embodiment, a fusion includes an in-frame fusion of an exon of Shootin-1 (KIAA1598, e.g., one more exons of KIAA1598 (e.g., one or more of exons 1-1111 of KIAA1598 or a fragment thereof, and an exon of C-Ros oncogene 1 (ROS1, e.g., one or more exons of a ROS1 (e.g., one or more of exons 36-43 of ROS1 or a fragment thereof. For example, the KIAA1598-ROS1 fusion can include an in-frame fusion within an intron of KIAA1598 (e.g., intron 11 or a fragment thereof, with an intron of ROS1 (e.g., intron 35 or a fragment thereof. In one embodiment, the fusion of the KIAA1598-ROS1 fusion comprises the nucleotide sequence of: chromosome 10 at one or more of a nucleotide (plus or minus 10, 20, 30, 50, 60, 70, 80, 100 or more nucleotides) and chromosome 6 at one or more of a nucleotide (plus or minus 10, 20, 30, 50, 60, 70, 80, 100 or more nucleotides). In one embodiment, the KIAA1598-ROS1 fusion is a translocation, e.g., a translocation of a portion of chromosome 6 and a portion of chromosome 6.

In certain embodiments, the KIAA1598-ROS1 fusion is in a 5′-KIAA1598 to 3′-ROS1 configuration (also referred to herein as “5′-KIAA1598-ROS1-3′).” The term “fusion” or “fusion molecule” can refer to a polypeptide or a nucleic acid fusion, depending on the context. It may include a full-length sequence of a fusion or a fragment thereof, e.g., a fusion junction (e.g., a fragment including a portion of KIAA1598 and a portion of ROS1, e.g., a portion of the KIAA1598-ROS1 fusion described herein). In one embodiment, the KIAA1598-ROS1 fusion polypeptide includes a fragment of the amino acid sequence shown in SEQ ID NO:65 and a fragment of the amino acid sequence shown in SEQ ID NO:12, or an amino acid sequence substantially identical thereto. In another embodiment, the KIAA1598-ROS1 fusion nucleic acid includes a fragment of the nucleotide sequence shown in SEQ ID NO:64 and a fragment of the nucleotide sequence shown in SEQ ID NO: 11, or a nucleotide sequence substantially identical thereto. In one embodiment, the KIAA1598-ROS1 fusion polypeptide comprises sufficient KIAA1598 and sufficient ROS1 sequence such that the 5′ KIAA1598-3′ ROS1 fusion has kinase activity, e.g., has elevated activity. e.g., tyrosine kinase activity. In any event, the fusion causes activation in the cells harboring it of oncogenic signaling pathways.

In certain embodiments, the KIAA1598-ROS1 fusion comprises one or more (or all of) exons 1-11 from KIAA1598 and one or more (or all of) exons 36-43 of ROS1 (e.g., one or more of the exons shown in SEQ ID NO:64 and SEQ ID NO:11. In another embodiment, the KIAA1598-ROS1 fusion comprises one or more (or all of) exons 1-11 of KIAA1598 and one or more (or all of) exons 36-43 of ROS1. In certain embodiments, the KIAA1598-ROS1 fusion comprises at least 1, 2, 3, 4, 5, 6, 7, 8, 9, 10, 11 or more exons (or encoded exons) from KIAA1598 and at least 1, 2, 3, 4, 5, 6, 7, 8 or more exons (or encoded exons) from ROS1 (e.g., from the KIAA1598 and ROS1 sequences shown in SEQ ID NO:64 and SEQ ID NO:65 and SEQ ID NO: 11 and SEQ ID NO: 12.

In certain embodiments, the KIAA1598-ROS1 fusion comprises exons 1-11 or a fragment thereof from KIAA1598, and exons 36-43 or a fragment thereof from ROS1 (e.g., as shown in SEQ ID NO:64 and SEQ ID NO:11. In one embodiment, the KIAA1598-ROS1 fusion comprises at least 5, 10, 15, 20, 30, 40, 50 or more amino acids from exons 1-11 of KIAA1598 (e.g., from the amino acid sequence of KIAA1598 as shown in SEQ ID NO:65 (e.g., from the amino acid sequence of KIAA1598 preceding the fusion junction with ROS1, and at least 5, 10, 15, 20, 30, 40, 50 or more amino acids from exons 36-43 of ROS1 (e.g., from the amino acid sequence of ROS1 as shown in SEQ ID NO:12. In another embodiment, the KIAA1598-ROS1 fusion comprises at least 6, 12, 15, 20, 25, 50, 75, 100 or more nucleotides from exons 1-11 of KIAA1598 (e.g., from the nucleotide sequence of KIAA1598 as shown in SEQ ID NO:64 (e.g., from the nucleotide sequence of KIAA1598 preceding the fusion junction with ROS1; and at least 6, 12, 15, 20, 25, 50, 75, 100 or more nucleotides from exons 36-43 of ROS1 (e.g., from the nucleotide sequence of ROS1 as shown in SEQ ID NO: 11.

### KIAA1598-ROS1 Nucleic Acid Molecules

In one aspect, the invention features a nucleic acid molecule (e.g., an isolated or purified) nucleic acid molecule that includes a fragment of a KIAA1598 gene and a fragment of a ROS1 gene. In one embodiment, the nucleotide sequence encodes a KIAA1598-ROS1 fusion polypeptide that includes a tyrosine kinase domain or a functional fragment thereof. In another embodiment, the nucleotide sequence encodes a fragment of the ROS1 polypeptide including the amino acid sequence of SEQ ID NO: 12 or a fragment thereof, or a sequence substantially identical thereto. In other embodiments, the nucleic acid molecule includes a fragment of the KIAA1598 gene encoding the amino acid sequence of SEQ ID NO:65 or a fragment thereof, or a sequence substantially identical thereto. In yet other embodiments, the nucleic acid molecule includes a nucleotide sequence encoding the amino acid sequence shown in SEQ ID NO:65, or a fragment thereof, and the amino acid sequence shown in SEQ ID NO:12 or a fragment thereof, or a sequence substantially identical thereto.

In one embodiment, the nucleic acid molecule includes a fusion, e.g., an in-frame fusion, between an intron of KIAA1598 (e.g., intron 11, or a fragment thereof), and an intron of ROS1 (e.g., intron 35, or a fragment thereof). The KIAA1598-ROS1 fusion can comprise a fusion of the nucleotide sequence of: chromosome 10 at one or more of a nucleotide (plus or minus 10, 20, 30, 50, 60, 70, 80, 100 nucleotides) and chromosome 6 at one or more of a nucleotide (plus or minus 10, 20, 30, 50, 60, 70, 80, 100 nucleotides), or a fragment thereof. In one embodiment, the KIAA1598-ROS1 fusion comprises a fusion of the nucleotide sequence of: chromosome 6 at one or more of a nucleotide (plus or minus 10, 20, 30, 50, 60, 70, 80, 100 nucleotides) and chromosome 6 at one or more of a nucleotide (plus or minus 10, 20, 30, 50, 60, 70, 80, 100 nucleotides), or a fragment thereof.

In another embodiment, the KIAA1598-ROS1 fusion comprises a nucleotide sequence (e.g., a fragment of a nucleotide sequence) shown in SEQ ID NO:64 and a nucleotide sequence (e.g., a fragment of a nucleotide sequence) shown in SEQ ID NO: 11, or a fragment of the fusion. In one embodiment, the KIAA1598-ROS1 fusion comprises a nucleotide sequence substantially identical to the nucleotide sequence (e.g., a fragment of a nucleotide sequence) shown in SEQ ID NO:64 and the nucleotide sequence (e.g., a fragment of a nucleotide sequence) shown SEQ ID NO: 11, or a fragment of the fusion. In one embodiment, the KIAA1598-ROS1 fusion comprises a nucleotide sequence at least 70%, at least 75%, at least 80%, at least 85%, at least 90%, at least 95%, at least 99%, at least 99.5 or greater, identical to the nucleotide sequence (e.g., a fragment of a nucleotide sequence) shown in SEQ ID NO:64 and to the nucleotide sequence (e.g., a fragment of a nucleotide sequence) shown in SEQ ID NO: 11. In one embodiment, the KIAA1598-ROS1 fusion comprises a nucleotide sequence containing at least 25, 50, 100, 150, 200, 500, 1000, 1500, 2000, 2500, 3000, or more nucleotides of the nucleotide sequence shown in SEQ ID NO:64 and a nucleotide sequence containing at least 25, 50, 100, 150, 200, 500, 1000, 1500, 2000, 2500, 3000, or more nucleotides of the nucleotide sequence shown in SEQ ID NO:11. In one embodiment, the KIAA1598-ROS1 fusion comprises a nucleotide sequence containing at least 25, 50, 100, 150, 200, 500, 1000, 1500, 2000, 2500, 3000, or more contiguous nucleotides of the nucleotide sequence shown in SEQ ID NO:64 and a nucleotide sequence containing at least 25, 50, 100, 150, 200, 500, 1000, 1500, 2000, 2500, 3000, or more nucleotides of the nucleotide sequence shown in SEQ ID NO: 11.

In another embodiment, the nucleic acid molecule includes a fusion, e.g., an in-frame fusion, of at least 1, 2, 3, 4, 5, 6, 7, 8, 9, 10, 11 or more exons of KIAA1598 or a fragment thereof (e.g., one or more of exons 1-11 of KIAA1598 or a fragment thereof), and at least 1, 2, 3, 4, 5, 6, 7, 8 or more exons of ROS1 or a fragment thereof (e.g., one or more of exons 36-43 of ROS1 or a fragment thereof). In yet other embodiments, the nucleic acid molecule includes a fragment the nucleotide sequence shown in SEQ ID NO:64 and a fragment of the nucleotide sequence shown in SEQ ID NO: 11 or a fragment of the fusion, or a sequence substantially identical thereto.

In one embodiment, the nucleic acid molecule is complementary to at least a portion of a nucleotide sequence disclosed herein, e.g., is capable of hybridizing under a stringency condition described herein to SEQ ID NO:64 and/or SEQ ID NO: 11, or a fragment thereof. In yet another embodiment, the nucleic acid molecule hybridizes to a nucleotide sequence that is complementary to at least a portion of a nucleotide sequence disclosed herein, e.g., is capable of hybridizing under a stringency condition to a nucleotide sequence complementary to SEQ ID NO:64 and/or SEQ ID NO: 11, or a fragment thereof. The nucleotide sequence of a cDNA encoding an exemplary 5′ KIAA1598-3′ ROS1 fusion is shown in at least exon 11 (e.g., exons 1-11 of SEQ ID NO:64 and at least exon 36 (e.g., exons 36-43 of SEQ ID NO: 11, and the predicted amino acid sequence is shown in the corresponding encoded exons of SEQ ID NO:65 and the corresponding encoded exons of SEQ ID NO: 12, respectively.

In an embodiment the KIAA1598-ROS1 nucleic acid molecule comprises sufficient KIAA1598 and sufficient ROS1 sequence such that the encoded 5′ KIAA1598-3′ ROS1 fusion has kinase activity, e.g., has elevated activity. In any event, the fusion causes activation in the cells harboring it of oncogenic signaling pathways. In certain embodiments, the 5′ KIAA1598-3′ ROS1 fusion comprises exons 1-11 from KIAA1598 and exons 36-43 from ROS1. In certain embodiments, the KIAA1598-ROS1 fusion comprises at least 1, 2, 3, 4, 5, 6, 7, 8, 9, 10, 11 or more exons from KIAA1598 and at least at least 1, 2, 3, 4, 5, 6, 7, 8 or more exons of ROS1. In certain embodiments, the KIAA1598-ROS1 fusion comprises a fusion of exon 11 from KIAA1598 and exon 36 from ROS1. In another embodiment, the KIAA1598-ROS1 fusion comprises 1, 2, 3, 4, 5, 6, 7, 8, 9, 10, 11 or more exons of KIAA1598; and at least 1, 2, 3, 4, 5, 6, 7, 8 or more exons of ROS1.

In one embodiment, the nucleic acid molecule includes a nucleotide sequence that has an in-frame fusion of intron 11 of KIAA1598 (e.g., NM 178039 with intron 36 of ROS1 (e.g., NM 002944. In another embodiment, the nucleic acid molecule includes a nucleotide sequence that includes a breakpoint. For example, the nucleic acid molecule includes a nucleotide sequence that includes the fusion junction between the KIAA1598 gene and the ROS1 gene, e.g., the breakpoint between intron 11 of KIAA1598 and intron 35 of ROS1. In other embodiments, the nucleic acid molecules includes a nucleotide sequence of one or more of nucleotide of chromosome 6 coupled to (e.g., directly or indirectly juxtaposed to) one or more of nucleotide of chromosome 6. In one embodiment, the nucleic acid molecule is complementary to at least a portion of a nucleotide sequence disclosed herein, e.g., is capable of hybridizing under a stringency condition described herein to SEQ ID NO:64 and/or SEQ ID NO: 11 or a fragment thereof. In yet other embodiment, the nucleic acid molecule hybridizes to a nucleotide sequence that is complementary to at least a portion of a nucleotide sequence disclosed herein, e.g., is capable of hybridizing under a stringency condition described herein to a nucleotide sequence complementary to SEQ ID NO:64 or SEQ ID NO: 11 or a fragment thereof.

In another embodiment, the KIAA1598-ROS1 fusion nucleic acid comprises at least 6, 12, 15, 20, 25, 50, 75, 100 or more nucleotides from exon 11 of KIAA1598 (e.g., from the nucleotide sequence of KIAA1598 preceding the fusion junction with ROS1, e.g., of the KIAA1598 sequence shown in SEQ ID NO:64, and at least 6, 12, 15, 20, 25, 50, 75, 100 or more nucleotides from exon 36 of ROS1 (e.g., from the nucleotide sequence of ROS1 following the fusion junction with KIAA1598, e.g., of the ROS1 sequence shown in SEQ ID NO: 11.

In other embodiments, the nucleic acid molecule includes a nucleotide sequence encoding a KIAA1598-ROS1 fusion polypeptide that includes a fragment of a KIAA1598 gene and a fragment of a ROS1 gene. In one embodiment, the nucleotide sequence encodes an KIAA1598-ROS1 fusion polypeptide that includes e.g., a tyrosine kinase domain or a functional fragment thereof. In yet other embodiments, the nucleic acid molecule includes a nucleotide sequence encoding the amino acid sequence (e.g., a fragment of the amino acid sequence) shown in SEQ ID NO:65 and a nucleotide sequence encoding the amino acid sequence (e.g., a fragment of the amino acid sequence) shown in SEQ ID NO: 12, or a fragment of the fusion, or a sequence substantially identical thereto. In one embodiment, the encoded KIAA1598-ROS11 fusion polypeptide includes a tyrosine kinase domain or a functional fragment thereof.

In a related aspect, the invention features nucleic acid constructs that include the KIAA1598-ROS1 nucleic acid molecules described herein. In certain embodiments, the nucleic acid molecules are operatively linked to a native or a heterologous regulatory sequence. Also included are vectors and host cells that include the KIAA1598-ROS1 nucleic acid molecules described herein, e.g., vectors and host cells suitable for producing the nucleic acid molecules and polypeptides described herein.

In a related aspect, methods of producing the nucleic acid molecules and polypeptides described herein are also described.

In another aspect, the invention features nucleic acid molecules that reduce or inhibit the expression of a nucleic acid molecule that encodes a KIAA1598-ROS11 fusion described herein. Examples of such nucleic acid molecules include, for example, antisense molecules, ribozymes, RNAi, triple helix molecules that hybridize to a nucleic acid encoding KIAA1598-ROS1, or a transcription regulatory region of KIAA1598-ROS1, and blocks or reduces mRNA expression of KIAA1598-ROS1.

### Nucleic Acid Detection and Capturing Reagents

The invention also features a nucleic acid molecule, e.g., nucleic acid fragment, suitable as probe, primer, bait or library member that includes, flanks, or hybridizes to the KIAA1598-ROS1 fusions described herein, which is useful for identifying, or is otherwise based on, the KIAA1598-ROS1 fusions described herein. In certain embodiments, the probe, primer or bait molecule is an oligonucleotide that allows capture, detection or isolation of a KIAA1598-ROS1 fusion nucleic acid molecule described herein. The oligonucleotide can comprise a nucleotide sequence substantially complementary to a fragment of the KIAA1598-ROS1 fusion nucleic acid molecules described herein. The sequence identity between the nucleic acid fragment, e.g., the oligonucleotide, and the target KIAA1598-ROS1 sequence need not be exact, so long as the sequences are sufficiently complementary to allow the capture, detection or isolation of the target sequence. In one embodiment, the nucleic acid fragment is a probe or primer that includes an oligonucleotide between about 5 and 25, e.g., between 10 and 20, or 10 and 15 nucleotides in length. In other embodiments, the nucleic acid fragment is a bait that includes an oligonucleotide between about 100 to 300 nucleotides, 204 and 230 nucleotides, or 150 and 200 nucleotides, in length.

In one embodiment, the nucleic acid fragment can be used to identify or capture. e.g., by hybridization, a KIAA1598-ROS1 fusion. For example, the nucleic acid fragment can be a probe, a primer, or a bait, for use in identifying or capturing, e.g., by hybridization, a KIAA1598-ROS1 fusion described herein. In one embodiment, the nucleic acid fragment can be useful for identifying or capturing a KIAA1598-ROS1 breakpoint, e.g., the nucleotide sequence of: chromosome 10 at nucleotide plus or minus 10, 20, 30, 40, 50, 60, 80, 100, 150 nucleotides and chromosome 6 at nucleotide plus or minus 10, 20, 30, 40 50, 60, 80, 100, 150 nucleotides.

In one embodiment, the nucleic acid fragment hybridizes to a nucleotide sequence within a chromosomal rearrangement that creates an in-frame fusion of intron 11 of KIAA1598 with intron 35 of ROS1. In one embodiment, the nucleic acid fragment hybridizes to a nucleotide sequence in the fusion region. In other embodiments, the nucleic acid molecules includes a nucleotide sequence in the region of one nucleotide sequence of chromosome 6 coupled to (e.g., juxtaposed to) nucleotides in the region of another nucleotide sequence of chromosome 6. In one embodiment, the nucleic acid fragment hybridizes to a nucleotide sequence that includes a breakpoint, e.g., the nucleotide sequence of: chromosome 10 at nucleotide plus or minus 10, 20, 30, 40, 50, 60, 80, 100, 150 or more nucleotides and chromosome 6 at nucleotide plus or minus 10, 20, 30, 40, 50, 60, 80, 100, 150 or more nucleotides. For example, the nucleic acid fragment can hybridize to a nucleotide sequence that includes the fusion junction between the KIAA1598 gene and the ROS1 gene, e.g., a nucleotide sequence that includes a portion of a nucleotide sequence within intron 11 of an KIAA1598 gene and intron 35 of a ROS1 gene.

In another embodiment, the nucleic acid fragment hybridizes to a nucleotide sequence that comprises at least 6, 12, 15, 20, 25, 50, 75, 100, 150 or more nucleotides from exon 11 of KIAA1598 (e.g., from the nucleotide sequence of KIAA1598 preceding the fusion junction with ROS1, e.g., of the KIAA1598 sequence shown in SEQ ID NO:64, and at least 6, 12, 15, 20, 25, 50, 75, 100, 150 or more nucleotides from exon 36 of ROS1 (e.g., from the nucleotide sequence of ROS1 following the fusion junction with KIAA1598, e.g., of the ROS1 sequence shown in SEQ ID NO: 11.

In one embodiment, the KIAA1598-ROS1 fusion includes the full sequence or a fragment of the nucleotide sequence shown in SEQ ID NO:74, or a nucleotide sequence substantially identical thereto. In another embodiment, the KIAA1598-ROS1 fusion polypeptide includes the full sequence or a fragment of the amino acid sequence shown in SEQ ID NO:75, or an amino acid sequence substantially identical thereto.

The probes or primers described herein can be used, for example, for FISH detection or PCR amplification. In one exemplary embodiment where detection is based on PCR, amplification of the KIAA1598-ROS1 fusion junction fusion junction can be performed using a primer or a primer pair, e.g., for amplifying a sequence flanking the fusion junctions described herein, e.g., the mutations or the junction of a chromosomal rearrangement described herein, e.g., KIAA1598-ROS1.

In one embodiment, a pair of isolated oligonucleotide primers can amplify a region containing or adjacent to a position in the KIAA1598-ROS1 fusion. For example, forward primers can be designed to hybridize to a nucleotide sequence within KIAA1598 genomic or mRNA sequence (e.g., a nucleotide sequence within exon 11 of KIAA1598 of SEQ ID NO:64, and the reverse primers can be designed to hybridize to a nucleotide sequence of ROS1 (e.g., a nucleotide sequence within exon 36 of ROS1, of SEQ ID NO:11.

In another embodiment, the nucleic acid fragments can be used to identify, e.g., by hybridization, a KIAA1598-ROS1 fusion. In one embodiment, the nucleic acid fragment hybridizes to a nucleotide sequence that includes a fusion junction between the KIAA1598 transcript and the ROS1 transcript.

In other embodiments, the nucleic acid fragment includes a bait that comprises a nucleotide sequence that hybridizes to a KIAA1598-ROS1 fusion nucleic acid molecule described herein, and thereby allows the capture or isolation of said nucleic acid molecule. In one embodiment, a bait is suitable for solution phase hybridization. In other embodiments, a bait includes a binding entity, e.g., an affinity tag, that allows capture and separation, e.g., by binding to a binding entity, of a hybrid formed by a bait and a nucleic acid hybridized to the bait.

In other embodiments, the nucleic acid fragment includes a library member comprising a KIAA1598-ROS1 nucleic acid molecule described herein. In one embodiment, the library member includes a rearrangement that results in the KIAA1598-ROS1 fusion described herein.

The nucleic acid fragment can be detectably labeled with, e.g., a radiolabel, a fluorescent label, a bioluminescent label, a chemiluminescent label, an enzyme label, a binding pair label, or can include an affinity tag; a tag, or identifier (e.g., an adaptor, barcode or other sequence identifier).

### KIAA1598-ROS1 Fusion Polypeptides

In another embodiment, the KIAA1598-ROS1 fusion comprises an amino acid sequence (e.g., a fragment of the amino acid sequence) shown in SEQ ID NO:65 and an amino acid sequence (e.g., a fragment of the amino acid sequence) shown in SEQ ID NO: 12, or a fragment of the fusion. In one embodiment, the KIAA1598-ROS1 fusion comprises an amino acid sequence substantially identical to the amino acid sequence (e.g., a fragment of the amino acid sequence) shown in SEQ ID NO:65 and the amino acid sequence (e.g., a fragment of the amino acid sequence) shown in SEQ ID NO:12, or a fragment thereof. In one embodiment, the KIAA1598-ROS1 fusion comprises an amino acid sequence at least 70%, at least 75%, at least 80%, at least 85%, at least 90%, at least 95%, at least 99%, or at least 99.5 identical to the amino acid sequence (e.g., a fragment of the amino acid sequence) shown in SEQ ID NO:65 and the amino acid sequence (e.g., a fragment of the amino acid sequence) shown in SEQ ID NO:12. In one embodiment, the KIAA1598-ROS1 fusion comprises a sequence containing at least 10, 20, 50, 100, 500, 600, 700, 800, 900, 1000, or more amino acids of the amino acid sequence shown in SEQ ID NO:65 and SEQ ID NO: 12. In one embodiment, the KIAA1598-ROS1 fusion comprises an amino acid sequence containing at least 5, 10, 20, 50, 100, 500, 600, 700, 800, 900, 1000, or more contiguous amino acids of the amino acid sequence shown in SEQ ID NO:65 and at least 5, 10, 20, 50, 100, 500, 600, 700, 800, 900, 1000, or more contiguous amino acids of the amino acid sequence shown in SEQ ID NO:12. In one embodiment, the 5′ KIAA1598-3′ ROS1 fusion polypeptide includes a receptor tyrosine kinase domain or a functional fragment thereof. In an embodiment, the 5′KIAA1598-3′ROS1 fusion polypeptide comprises sufficient ROS1 and sufficient KIAA1598 sequence such that it has kinase activity, e.g., has elevated activity. In any event, the fusion causes activation in the cells harboring it of oncogenic signaling pathways.

In another aspect, the invention features a KIAA598-ROS1 fusion polypeptide (e.g., a purified KIAA1598-ROS1 fusion polypeptide), a biologically active or antigenic fragment thereof, as well as reagents (e.g., antibody molecules that bind to a KIAA1598-ROS1 fusion polypeptide), methods for modulating a KIAA1598-ROS1 polypeptide activity and detection of a KIAA1598-ROS1 polypeptide.

In one embodiment, the KIAA1598-ROS1 fusion polypeptide has at least one biological activity. In one embodiment, at least one biological activity of the KIAA1598-ROS1 fusion polypeptide is reduced or inhibited by an anti-cancer drug, e.g., a KIAA1598 inhibitor, a ROS1 inhibitor. In one embodiment, at least one biological activity of the KIAA1598-ROS1 fusion polypeptide is reduced or inhibited by a ROS1 inhibitor. In one embodiment, at least one biological activity of the KIAA1598-ROS1 fusion polypeptide is reduced or inhibited by a KIAA1598 inhibitor. In one embodiment, at least one biological activity of the KIAA1598-ROS1 fusion polypeptide is reduced or inhibited by a ROS1 inhibitor, e.g., Ganetespib; Crizotinib; TAE684; a dual ALK and ROS1 inhibitor.

In yet other embodiments, the KIAA1598-ROS1 fusion polypeptide is encoded by a nucleic acid molecule described herein. In one embodiment, the KIAA1598-ROS1 fusion polypeptide is encoded by an in-frame fusion of intron 11 of KIAA1598 with intron 35 of ROS1 (e.g., a sequence on chromosome 10 and a sequence on chromosome 6. In another embodiment, the KIAA1598-ROS1 fusion polypeptide includes an amino acid sequence encoded by a nucleotide sequence comprising a fusion junction between the KIAA1598 transcript and the ROS1 transcript.

In certain embodiments, the KIAA1598-ROS1 fusion polypeptide comprises one or more of encoded exons 1-11 from KIAA1598 and one or more of encoded exons 36-43 of ROS1. In certain embodiments, the KIAA1598-ROS1 fusion polypeptide comprises at least 1, 2, 3, 4, 5, 6, 7, 8, 9, 10, 11 or more encoded exons of KIAA1598 and at least at least 1, 2, 3, 4, 5, 6, 7, 8 or more encoded exons of ROS1. In certain embodiments, the KIAA1598-ROS1 fusion polypeptide comprises a fusion of encoded exon 11 from KIAA1598 and encoded exon 36 from ROS1 (or a fragment thereof). In other embodiments, the fusion comprises at least 1, 2, 3, 4, 5, 6, 7, 8, 9, 10, 11 or more encoded exons of KIAA1598; and at least 1, 2, 3, 4, 5, 6, 7, 8 or more encoded exons of ROS1. In certain embodiments, the KIAA1598-ROS1 fusion polypeptide comprises encoded exons 1-11 from KIAA1598 and exons 36-43 of ROS1. In certain embodiments, the 5′ KIAA1598-3′ ROS1 fusion polypeptide comprises a fusion junction of the sequence of exon 11 from KIAA1598 and the sequence of exon 36 from ROS1.

In certain embodiments, the KIAA1598-ROS1 fusion comprises the amino acid sequence corresponding to exon 11 or a fragment thereof from KIAA1598, and the amino acid sequence corresponding to exon 36 or a fragment thereof from ROS1 (e.g., as shown in SEQ ID NO:65 and SEQ ID NO:12. In one embodiment, the KIAA1598-ROS1 fusion comprises at least 5, 10, 15, 20 or more amino acids from exon 11 of KIAA1598 (e.g., from the amino acid sequence of KIAA1598 preceding the fusion junction with ROS1, e.g., of the KIAA1598 sequence shown in EQ ID NO:65, and at least 5, 10, 15, 20 or more amino acids from exon 36 of ROS1 (e.g., from the amino acid sequence of ROS1 following the fusion junction with KIAA1598, e.g., of the ROS1 sequence shown in SEQ ID NO: 12.

In one embodiment, the KIAA1598-ROS1 fusion polypeptide includes a tyrosine kinase domain or a functional fragment thereof. In a related aspect, the invention features KIAA1598-ROS1 fusion polypeptide or fragments operatively linked to heterologous polypeptides to form fusion proteins.

In another embodiment, the KIAA1598-ROS1 fusion polypeptide or fragment is a peptide, e.g., an immunogenic peptide or protein. The peptide contains a fusion junction described herein. Such immunogenic peptides or proteins can be used to raise antibodies specific to the fusion protein. In other embodiments, such immunogenic peptides or proteins can be used for vaccine preparation. The vaccine preparation can include other components, e.g., an adjuvant.

In another aspect, the invention features antibody molecules that bind to a KIAA1598-ROS1 fusion polypeptide or fragment described herein. In such embodiments, the antibody can be used as a reagent to distinguish wild type ROS1 (or KIAA1598 from KIAA1598-ROS1.

### Detection Reagents and Detection of Mutations

In another aspect, the invention features a detection reagent, e.g., a purified or an isolated preparation thereof. Detection reagents can distinguish a nucleic acid, or protein sequence, having a breakpoint, e.g., a KIAA1598-ROS1 breakpoint; from a reference sequence. In one embodiment, the detection reagent detects (e.g., specifically detects) a KIAA1598-ROS1 fusion nucleic acid or a polypeptide, e.g., distinguishes a wild type ROS1 or another ROS1 fusion (or KIAA1598) from a KIAA1598-ROS1 nucleic acid (e.g., as described herein in SEQ ID NO:64 and SEQ ID NO:12); or a KIAA1598-ROS1 polypeptide (e.g., as described herein in SEQ ID NO:65 and SEQ ID NO:12).

Detection reagents, e.g., nucleic acid-based detection reagents, can be used to identify mutations in a target nucleic acid, e.g., DNA, e.g., genomic DNA or cDNA, or RNA, e.g., in a sample, e.g., a sample of nucleic acid derived from a neoplastic or tumor cell, e.g., a melanocytic neoplasm, melanoma or metastatic cell. Detection reagents, e.g., antibody-based detection reagents, can be used to identify mutations in a target protein, e.g., in a sample, e.g., a sample of protein derived from, or produced by, a neoplastic or tumor cell, e.g., a melanocytic neoplasm, melanoma or metastatic cell.

### Method of Treatment

In a related aspect, a method of inhibiting, reducing, or treating a hyperproliferative disorder, e.g., a neoplasm (including benign, pre-malignant or malignant (e.g., a cancer), in a subject is provided. The method includes administering to the subject a preselected therapeutic agent. e.g., an anti-cancer agent (e.g., a kinase inhibitor), as a single agent, or in combination with other active agents, in an amount sufficient to reduce, inhibit or treat the activity or expression of KIAA1598-ROS1 (e.g., an KIAA1598-ROS1 fusion described herein), thereby inhibiting, reducing, or treating the hyperproliferative disorder in the subject.

In one embodiment, the subject treated has a KIAA1598-ROS1 fusion; e.g., the subject has a tumor or cancer harboring a KIAA1598-ROS1 fusion. In other embodiments, the subject has been previously identified as having an KIAA1598-ROS1 fusion. In yet other embodiments, the subject has been previously identified as being likely or unlikely to respond to treatment with a protein kinase inhibitor, e.g., a subject that has previously participated in a clinical trial. In other embodiments, the subject has been previously identified as being likely or unlikely to respond to treatment with a protein kinase inhibitor, based on the presence of the KIAA1598-ROS1 fusion. In one embodiment, the subject is a mammal, e.g., a human. In one embodiment, the subject has, or at risk of having a cancer at any stage of disease. In other embodiments, the subject is a patient, e.g., a cancer patient.

In certain embodiments, the neoplasm or neoplastic cell is a benign, pre-malignant, malignant (cancer) or metastasis. In certain embodiments, the cancer is a solid tumor, a soft tissue tumor, or a metastatic lesion.

In one embodiment, the neoplasm or neoplastic cell is a benign, pre-malignant, malignant (cancer) or metastasis. In certain embodiments, the cancer is a solid tumor, a soft tissue tumor, or a metastatic lesion. In one embodiment, the cancer is a carcinoma. In one embodiment, the cancer is chosen from a lung cancer, a pancreatic cancer, melanoma, a colorectal cancer, an esophageal-gastric cancer, a thyroid cancer, or an adenocarcinoma. In one embodiment, the cancer is a lung adenocarcinoma. In other embodiment, the lung cancer is chosen from one or more of the following: non-small cell lung cancer (NSCLC), small cell lung cancer SCLC), squamous cell carcinoma SCC), adenocarcinoma of the lung, bronchogenic carcinoma, or a combination thereof. In one embodiment, the lung cancer is NSCLC or SCC.

In one embodiment, the anti-cancer agent is a ROS1 inhibitor. In one embodiment, the anti-cancer agent is a KIAA1598 inhibitor. In one embodiment, the anti-cancer agent is a kinase inhibitor. In one embodiment, the anti-cancer agent is a ROS1 inhibitor, e.g., Ganetespib; Crizotinib; TAE684; a dual ALK and ROS1 inhibitor.

### DCTN1-ALK Fusions

In one embodiment, a fusion includes an in-frame fusion of an exon of Dynactin subunit 1 (DCTN1), e.g., one more exons of DCTN1, particularly one or more of exons 1-26 of DCTN1 or a fragment thereof, and an exon of anaplastic lymphoma receptor tyrosine kinase (ALK), e.g., one or more exons of an ALK such as one or more of exons 20-29 of ALK or a fragment thereof. For example, the DCTN1-ALK fusion can include an in-frame fusion within an intron of DCTN1 (e.g., intron 26 or a fragment thereof, with an intron of ALK (e.g., intron 19 or a fragment thereof. In one embodiment, the fusion of the DCTN1-ALK fusion comprises a nucleotide sequence of chromosome 2 at one or more of a nucleotide (plus or minus 10, 20, 30, 50, 60, 70, 80, 100 or more nucleotides) and another nucleotide sequence of chromosome 2 at one or more of a nucleotide (plus or minus 10, 20, 30, 50, 60, 70, 80, 100 or more nucleotides). In one embodiment, the DCTN1-ALK fusion is a translocation, e.g., a translocation of one portion of chromosome 2 and another portion of chromosome 2.

In certain embodiments, the DCTN1-ALK fusion is in a 5′-DCTN1 to 3′-ALK configuration (also referred to herein as “5′-DCTN1-ALK-3′).” The term “fusion” or “fusion molecule” can refer to a polypeptide or a nucleic acid fusion, depending on the context. It may include a full-length sequence of a fusion or a fragment thereof, e.g., a fusion junction (e.g., a fragment including a portion of DCTN1 and a portion of ALK, e.g., a portion of the DCTN1-ALK fusion described herein). In one embodiment, the DCTN1-ALK fusion polypeptide includes a fragment of the amino acid sequence shown in SEQ ID NO:67 and a fragment of the amino acid sequence shown in SEQ ID NO:7, or an amino acid sequence substantially identical thereto. In another embodiment, the DCTN1-ALK fusion nucleic acid includes a fragment of the nucleotide sequence shown in SEQ ID NO:66 and a fragment of the nucleotide sequence shown in SEQ ID NO:7, or a nucleotide sequence substantially identical thereto. In one embodiment, the DCTN1-ALK fusion polypeptide comprises sufficient DCTN1 and sufficient ALK sequence such that the 5′ DCTN1-3′ ALK fusion has kinase activity, e.g., has elevated activity, e.g., tyrosine kinase activity. In any event, the fusion causes activation in the cells harboring it of oncogenic signaling pathways.

In certain embodiments, the DCTN1-ALK fusion comprises one or more (or all of) exons 1-26 from DCTN1 and one or more (or all of) exons 20-29 of ALK (e.g., one or more of the exons shown in SEQ ID NO:66 and SEQ ID NO:7). In another embodiment, the DCTN1-ALK fusion comprises one or more (or all of) exons 1-26 of DCTN1 and one or more (or all of) exons 20-29 of ALK. In certain embodiments, the DCTN1-ALK fusion comprises at least 1, 2, 3, 4, 5, 6, 7, 8, 9, 10, 11, 12, 13, 14, 15, 16, 17, 18, 19, 20, 21, 22, 23, 24, 25, 26 or more exons (or encoded exons) from DCTN1 and at least 1, 2, 3, 4, 5, 6, 7, 8, 4, 5, 6, 7, 8, 9, 10 or more exons (or encoded exons) from ALK (e.g., from the DCTN1 and ALK sequences shown in SEQ ID NO:66 and SEQ ID NO:67 and SEQ ID NO:7 and SEQ ID NO:8).

In certain embodiments, the DCTN1-ALK fusion comprises exons 1-26 or a fragment thereof from DCTN1, and exons 20-29 or a fragment thereof from ALK (e.g., as shown in SEQ ID NO:66 and SEQ ID NO:7). In one embodiment, the DCTN1-ALK fusion comprises at least 5, 10, 15, 20, 30, 40, 50 or more amino acids from exons 1-26 of DCTN1 (e.g., from the amino acid sequence of DCTN1 as shown in SEQ ID NO:67, particularly from the amino acid sequence of DCTN1 preceding the fusion junction with ALK), and at least 5, 10, 15, 20, 30, 40, 50 or more amino acids from exons 20-29 of ALK (e.g., from the amino acid sequence of ALK as shown in SEQ ID NO:7). In another embodiment, the DCTN1-ALK fusion comprises at least 6, 12, 15, 20, 25, 50, 75, 100 or more nucleotides from exons 1-26 of DCTN1 (e.g., from the nucleotide sequence of DCTN1 as shown in SEQ ID NO:66, particularly from the nucleotide sequence of DCTN1 preceding the fusion junction with ALK); and at least 6, 12, 15, 20, 25, 50, 75, 100 or more nucleotides from exons 20-29 of ALK (e.g., from the nucleotide sequence of ALK as shown in SEQ ID NO:7).

### DCTN1-ALK Nucleic Acid Molecules

In one aspect, the invention features a nucleic acid molecule (e.g., an isolated or purified) nucleic acid molecule that includes a fragment of a DCTN1 gene and a fragment of an ALK gene. In one embodiment, the nucleotide sequence encodes a DCTN1-ALK fusion polypeptide that includes a tyrosine kinase domain or a functional fragment thereof. In another embodiment, the nucleotide sequence encodes a fragment of the ALK polypeptide including the amino acid sequence of SEQ ID NO:7 or a fragment thereof, or a sequence substantially identical thereto. In other embodiments, the nucleic acid molecule includes a fragment of the DCTN1 polynucleotide encoding the amino acid sequence of SEQ ID NO:67 or a fragment thereof, or a sequence substantially identical thereto. In yet other embodiments, the nucleic acid molecule includes a nucleotide sequence encoding the amino acid sequence shown in 205 SEQ ID NO:67, or a fragment thereof, and the amino acid sequence shown in 62 SEQ ID NO:7 or a fragment thereof, or a sequence substantially identical thereto.

In one embodiment, the nucleic acid molecule includes a fusion. e.g., an in-frame fusion, between an intron of DCTN1 (e.g., intron 26, or a fragment thereof), and an intron of ALK (e.g., intron 19, or a fragment thereof). The DCTN1-ALK fusion can comprise a fusion of a nucleotide sequence of chromosome 2 at one or more of a nucleotide (plus or minus 10, 20, 30, 50, 60, 70, 80, 100 nucleotides) and another nucleotide sequence of chromosome 2 at one or more of a nucleotide (plus or minus 10, 20, 30, 50, 60, 70, 80, 100 nucleotides), or a fragment thereof. In one embodiment, the DCTN1-ALK fusion comprises a fusion of a nucleotide sequence of chromosome 2 at one or more of a nucleotide (plus or minus 10, 20, 30, 50, 60, 206, 80, 100 nucleotides) and another nucleotide sequence of chromosome 2 at one or more of a nucleotide (plus or minus 10, 20, 30, 50, 60, 70, 80, 100 nucleotides), or a fragment thereof.

In another embodiment, the DCTN1-ALK fusion comprises a nucleotide sequence (e.g., a fragment of a nucleotide sequence) shown in SEQ ID NO:66 and a nucleotide sequence (e.g., a fragment of a nucleotide sequence) shown in SEQ ID NO:7, or a fragment of the fusion itself. In one embodiment, the DCTN1-ALK fusion comprises a nucleotide sequence substantially identical to the nucleotide sequence (e.g., a fragment of a nucleotide sequence) shown in SEQ ID NO:66 and the nucleotide sequence (e.g., a fragment of a nucleotide sequence) shown SEQ ID NO:7, or a fragment of the fusion itself. In one embodiment, the DCTN1-ALK fusion comprises a nucleotide sequence at least 70%, at least 75%, at least 80%, at least 85%, at least 90%, at least 95%, at least 99%, at least 99.5 or greater, identical to the nucleotide sequence (e.g., a fragment of a nucleotide sequence) shown in 68 SEQ ID NO:66 and to the nucleotide sequence (e.g., a fragment of a nucleotide sequence) shown in 62 SEQ ID NO:7. In one embodiment, the DCTN1-ALK fusion comprises a nucleotide sequence containing at least 25, 50, 100, 150, 200, 500, 1000, 1500, 2000, 2500, 3000, or more nucleotides of the nucleotide sequence shown in SEQ ID NO:66 and a nucleotide sequence containing at least 25, 50, 100, 150, 200, 500, 1000, 1500, 2000, 2500, 3000, or more nucleotides of the nucleotide sequence shown in SEQ ID NO:7. In one embodiment, the DCTN1-ALK fusion comprises a nucleotide sequence containing at least 25, 50, 100, 150, 200, 500, 1000, 1500, 2000, 2500, 3000, or more contiguous nucleotides of the nucleotide sequence shown in SEQ ID NO:66 and a nucleotide sequence containing at least 25, 50, 100, 150, 200, 500, 1000, 1500, 2000, 2500, 3000, or more nucleotides of the nucleotide sequence shown in SEQ ID NO:7.

In another embodiment, the nucleic acid molecule includes a fusion, e.g., an in-frame fusion, of at least 1, 2, 3, 4, 5, 6, 7, 8, 9, 10, 11, 12, 13, 14, 15, 16, 17, 18, 19, 20, 21, 22, 23, 24, 25, 26 or more exons of DCTN1 or a fragment thereof (e.g., one or more of exons 1-26 of DCTN1 or a fragment thereof), and at least 1, 2, 3, 4, 5, 6, 7, 8, 4, 5, 6, 7, 8, 9, 10 or more exons of ALK or a fragment thereof (e.g., one or more of exons 20-29 of ALK or a fragment thereof). In yet other embodiments, the nucleic acid molecule includes a fragment of the nucleotide sequence shown in SEQ ID NO:66 and a fragment of the nucleotide sequence shown in SEQ ID NO:7 or a fragment of the fusion, or a sequence substantially identical thereto.

In one embodiment, the nucleic acid molecule is complementary to at least a portion of a nucleotide sequence disclosed herein, e.g., is capable of hybridizing under stringent conditions described herein to SEQ ID NO:66 and/or SEQ ID NO:7, or a fragment thereof. In yet another embodiment, the nucleic acid molecule hybridizes to a nucleotide sequence that is complementary to at least a portion of a nucleotide sequence disclosed herein, e.g., is capable of hybridizing under a stringent condition to a nucleotide sequence complementary to SEQ ID NO:66 and/or SEQ ID NO:7, or a fragment thereof. The nucleotide sequence of a cDNA encoding an exemplary 5′ DCTN1-3′ ALK fusion is shown in at least exons 26 (e.g., exons 1-26 of SEQ ID NO:66 and at least exon 20 (e.g., exons 20-29 of SEQ ID NO:7, and the predicted amino acid sequence is shown in the corresponding encoded exons of SEQ ID NO:67 and the corresponding encoded exons of SEQ ID NO:7, respectively.

In an embodiment the DCTN1-ALK nucleic acid molecule comprises sufficient DCTN1 and sufficient ALK sequence such that the encoded 5′ DCTN1-3′ ALK fusion has kinase activity, e.g., has elevated activity. In certain embodiments, the 5′ DCTN1-3′ ALK fusion comprises exons 1-26 from DCTN1 and exons 20-29 from ALK. In certain embodiments, the DCTN1-ALK fusion comprises at least 1, 2, 3, 4, 5, 6, 7, 8, 9, 10, 11, 12, 13, 14, 15, 16, 17, 18, 19, 20, 21, 22, 23, 24, 25, 26 or more exons from DCTN1 and at least at least 1, 2, 3, 4, 5, 6, 7, 8, 4, 5, 6, 7, 8, 9, 10 or more exons of ALK. In certain embodiments, the DCTN1-ALK fusion comprises a fusion of exons 26 from DCTN1 and exon 20 from ALK. In another embodiment, the DCTN1-ALK fusion comprises 1, 2, 3, 4, 5, 6, 7, 8, 9, 10, 11, 12, 13, 14, 15, 16, 17, 18, 19, 20, 21, 22, 23, 24, 25, 26 or more exons of DCTN1; and at least 1, 2, 3, 4, 5, 6, 7, 8, 4, 5, 6, 7, 8, 9, 10 or more exons of ALK.

In one embodiment, the nucleic acid molecule includes a nucleotide sequence that has an in-frame fusion of intron 26 of DCTN1 (e.g., NM_{—}004082 with intron 19 of ALK, e.g., NM_{—}004304). In another embodiment, the nucleic acid molecule includes a nucleotide sequence that includes a breakpoint. For example, the nucleic acid molecule includes a nucleotide sequence that includes the fusion junction between the DCTN1 gene and the ALK gene, e.g., the breakpoint between intron 26 of DCTN1 and intron 19 of ALK. In other embodiments, the nucleic acid molecules includes a nucleotide sequence of one or more of nucleotide of chromosome 2 coupled to (e.g., directly or indirectly juxtaposed to) one or more of nucleotide of chromosome 2. In one embodiment, the nucleic acid molecule includes the nucleotide sequence of: chromosome 2 at one or more of a nucleotide plus or minus 10, 20, 30, 40 50, 60, 80, 100, 150 nucleotides and chromosome 2 at one or more of a nucleotide plus or minus 10, 20, 30, 40 50, 60, 80, 100, 150 nucleotides (corresponding to the breakpoint of a DCTN1-ALK fusion), or a fragment thereof, or a sequence substantially identical thereto. In one embodiment, the nucleic acid molecule is complementary to at least a portion of a nucleotide sequence disclosed herein, e.g., is capable of hybridizing under stringent conditions described herein to SEQ ID NO:66 and/or SEQ ID NO:7 or a fragment thereof. In yet other embodiment, the nucleic acid molecule hybridizes to a nucleotide sequence that is complementary to at least a portion of a nucleotide sequence disclosed herein, e.g., is capable of hybridizing under a stringency condition described herein to a nucleotide sequence complementary to SEQ ID NO:66 or SEQ ID NO:7 or a fragment thereof.

In another embodiment, the DCTN1-ALK fusion nucleic acid comprises at least 6, 12, 15, 20, 25, 50, 75, 100 or more nucleotides from exons 26 of DCTN1 (e.g., from the nucleotide sequence of DCTN1 preceding the fusion junction with ALK, e.g., of the DCTN1 sequence shown in SEQ ID NO:66), and at least 6, 12, 15, 20, 25, 50, 75, 100 or more nucleotides from exon 20 of ALK (e.g., from the nucleotide sequence of ALK following the fusion junction with DCTN1, e.g., of the ALK sequence shown in SEQ ID NO:7).

In other embodiments, the nucleic acid molecule includes a nucleotide sequence encoding a DCTN1-ALK fusion polypeptide that includes a fragment of a DCTN1 gene and a fragment of an ALK gene. In one embodiment, the nucleotide sequence encodes a DCTN1-ALK fusion polypeptide that includes e.g., a tyrosine kinase domain or a functional fragment thereof. In yet other embodiments, the nucleic acid molecule includes a nucleotide sequence encoding the amino acid sequence (e.g., a fragment of the amino acid sequence) shown in SEQ ID NO:67 and a nucleotide sequence encoding the amino acid sequence (e.g., a fragment of the amino acid sequence) shown in SEQ ID NO:7, or a fragment of the fusion, or a sequence substantially identical thereto. In one embodiment, the encoded DCTN1-ALK fusion polypeptide includes a tyrosine kinase domain or a functional fragment thereof.

In a related aspect, the invention features nucleic acid constructs that include the DCTN1-ALK nucleic acid molecules described herein. In certain embodiments, the nucleic acid molecules are operatively linked to a native or a heterologous regulatory sequence. Also included are vectors and host cells that include the DCTN1-ALK nucleic acid molecules described herein, e.g., vectors and host cells suitable for producing the nucleic acid molecules and polypeptides described herein.

In a related aspect, methods of producing the nucleic acid molecules and polypeptides described herein are also described.

In another aspect, the invention features nucleic acid molecules that reduce or inhibit the expression of a nucleic acid molecule that encodes a DCTN1-ALK fusion described herein. Examples of such nucleic acid molecules include, for example, antisense molecules, ribozymes, RNAi, triple helix molecules that hybridize to a nucleic acid encoding DCTN1-ALK, or a transcription regulatory region of DCTN1-ALK, and blocks or reduces mRNA expression of DCTN1-ALK.

### Nucleic Acid Detection and Capturing Reagents

The invention also features a nucleic acid molecule, e.g., nucleic acid fragment, suitable as probe, primer, bait or library member that includes, flanks, or hybridizes to the DCTN1-ALK fusions described herein. Such probe primer bait or library member is useful for identifying, or is otherwise based on, the DCTN1-ALK fusions described herein. In certain embodiments, the probe, primer or bait molecule is an oligonucleotide that allows capture, detection or isolation of a DCTN1-ALK fusion nucleic acid molecule described herein. The oligonucleotide can comprise a nucleotide sequence substantially complementary to a fragment of the DCTN1-ALK fusion nucleic acid molecules described herein. The sequence identity between the nucleic acid fragment, e.g., the oligonucleotide, and the target DCTN1-ALK sequence need not be exact, so long as the sequences are sufficiently complementary to allow the capture, detection or isolation of the target sequence. In one embodiment, the nucleic acid fragment is a probe or primer that includes an oligonucleotide between about 5 and 25, e.g., between 10 and 20, or 10 and 15 nucleotides in length. In other embodiments, the nucleic acid fragment is a bait that includes an oligonucleotide between about 100 to 300 nucleotides, 130 and 230 nucleotides, or 150 and 200 nucleotides, in length.

In one embodiment, the nucleic acid fragment can be used to identify or capture, e.g., by hybridization, a DCTN1-ALK fusion. For example, the nucleic acid fragment can be a probe, a primer, or a bait, for use in identifying or capturing, e.g., by hybridization, a DCTN1-ALK fusion described herein. In one embodiment, the nucleic acid fragment can be useful for identifying or capturing a DCTN1-ALK breakpoint. e.g., the nucleotide sequence of: chromosome 2 at nucleotide plus or minus 10, 20, 30, 40, 50, 60, 80, 100, 150 nucleotides and chromosome 2 at nucleotide plus or minus 10, 20, 30, 40 50, 60, 80, 100, 150 nucleotides.

In one embodiment, the nucleic acid fragment hybridizes to a nucleotide sequence within a chromosomal rearrangement that creates an in-frame fusion of intron 26 of DCTN1 with intron 19 of ALK. In one embodiment, the nucleic acid fragment hybridizes to a nucleotide sequence in the fusion region. In other embodiments, the nucleic acid molecules includes a nucleotide sequence of chromosome 2 coupled to (e.g., juxtaposed to) nucleotides in another nucleotide sequence of chromosome 2. In one embodiment, the nucleic acid fragment hybridizes to a nucleotide sequence that includes a breakpoint, e.g., a nucleotide sequence of chromosome 2 at nucleotide plus or minus 10, 20, 30, 40, 50, 60, 80, 100, 150 or more nucleotides and chromosome 2 at nucleotide plus or minus 10, 20, 30, 40, 50, 60, 80, 100, 150 or more nucleotides. For example, the nucleic acid fragment can hybridize to a nucleotide sequence that includes the fusion junction between the DCTN1 gene and the ALK gene, e.g., a nucleotide sequence that includes a portion of a nucleotide sequence within intron 26 of a DCTN1 gene and intron 19 of an ALK gene.

In another embodiment, the nucleic acid fragment hybridizes to a nucleotide sequence that comprises at least 6, 12, 15, 20, 25, 50, 75, 100, 150 or more nucleotides from exons 26 of DCTN1 (e.g., from the nucleotide sequence of DCTN1 preceding the fusion junction with ALK, e.g., of the DCTN1 sequence shown in SEQ ID NO:66), and at least 6, 12, 15, 20, 25, 50, 75, 100, 150 or more nucleotides from exon 20 of ALK (e.g., from the nucleotide sequence of ALK following the fusion junction with DCTN1, e.g., of the ALK sequence shown in SEQ ID NO:7).

The probes or primers described herein can be used, for example, for FISH detection or PCR amplification. In one exemplary embodiment where detection is based on PCR, amplification of the DCTN1-ALK fusion junction fusion junction can be performed using a primer or a primer pair, e.g., for amplifying a sequence flanking the fusion junctions described herein, e.g., the mutations or the junction of a chromosomal rearrangement described herein, e.g., DCTN1-ALK.

In one embodiment, a pair of isolated oligonucleotide primers can amplify a region containing or adjacent to a position in the DCTN1-ALK fusion. For example, forward primers can be designed to hybridize to a nucleotide sequence within DCTN1 genomic or mRNA sequence (e.g., a nucleotide sequence within exons 26 of DCTN1 of SEQ ID NO:66), and the reverse primers can be designed to hybridize to a nucleotide sequence of ALK (e.g., a nucleotide sequence within exon 20 of ALK, of SEQ ID NO:7).

In another embodiment, the nucleic acid fragments can be used to identify. e.g., by hybridization, a DCTN1-ALK fusion. In one embodiment, the nucleic acid fragment hybridizes to a nucleotide sequence that includes a fusion junction between the DCTN1 transcript and the ALK transcript.

In other embodiments, the nucleic acid fragment includes a bait that comprises a nucleotide sequence that hybridizes to a DCTN1-ALK fusion nucleic acid molecule described herein, and thereby allows the capture or isolation said nucleic acid molecule. In one embodiment, a bait is suitable for solution phase hybridization. In other embodiments, a bait includes a binding entity, e.g., an affinity tag, that allows capture and separation, e.g., by binding to a binding entity, of a hybrid formed by a bait and a nucleic acid hybridized to the bait.

In other embodiments, the nucleic acid fragment includes a library member comprising a DCTN1-ALK nucleic acid molecule described herein. In one embodiment, the library member includes a rearrangement that results in a DCTN1-ALK fusion described herein.

The nucleic acid fragment can be detectably labeled with, e.g., a radiolabel, a fluorescent label, a bioluminescent label, a chemiluminescent label, an enzyme label, a binding pair label, or can include an affinity tag; a tag, or identifier (e.g., an adaptor, barcode or other sequence identifier).

### DCTN1-ALK Fusion Polypeptides

In another embodiment, the DCTN1-ALK fusion comprises an amino acid sequence (e.g., a fragment of the amino acid sequence) shown in SEQ ID NO:67 and an amino acid sequence (e.g., a fragment of the amino acid sequence) shown in SEQ ID NO:7, or a fragment of the fusion. In one embodiment, the DCTN1-ALK fusion comprises an amino acid sequence substantially identical to the amino acid sequence (e.g., a fragment of the amino acid sequence) shown in SEQ ID NO:67 and the amino acid sequence (e.g., a fragment of the amino acid sequence) shown in SEQ ID NO:7, or a fragment thereof. In one embodiment, the DCTN1-ALK fusion comprises an amino acid sequence at least 70%, at least 75%, at least 80%, at least 85%, at least 90%, at least 95%, at least 99%, at least 99.5 or greater, identical to the amino acid sequence (e.g., a fragment of the amino acid sequence) shown in SEQ ID NO:67 and the amino acid sequence (e.g., a fragment of the amino acid sequence) shown in 62 SEQ ID NO:7. In one embodiment, the DCTN1-ALK fusion comprises a sequence containing at least 10, 20, 50, 100, 500, 600, 700, 800, 900, 1000, or more amino acids of the amino acid sequence shown in SEQ ID NO:67 and SEQ ID NO:7. In one embodiment, the DCTN1-ALK fusion comprises an amino acid sequence containing at least 5, 10, 20, 50, 100, 500, 600, 700, 800, 900, 1000, or more contiguous amino acids of the amino acid sequence shown in SEQ ID NO:67 and at least 5, 10, 20, 50, 100, 500, 600, 700, 800, 900, 1000, or more contiguous amino acids of the amino acid sequence shown in SEQ ID NO:7. In one embodiment, the 5′ DCTN1-3′ ALK fusion polypeptide includes a receptor tyrosine kinase domain or a functional fragment thereof. In an embodiment, the 5′DCTN1-3′ALK fusion polypeptide comprises sufficient ALK and sufficient DCTN1 sequence such that it has kinase activity, e.g., has elevated activity.

In another aspect, the invention features a DCTN1-ALK fusion polypeptide (e.g., a purified DCTN1-ALK fusion polypeptide), a biologically active or antigenic fragment thereof, as well as reagents (e.g., antibody molecules that bind to a DCTN1-ALK fusion polypeptide), methods for modulating a DCTN1-ALK polypeptide activity and detection of a DCTN1-ALK polypeptide.

In one embodiment, the DCTN1-ALK fusion polypeptide has at least one biological activity. In one embodiment, at least one biological activity of the DCTN1-ALK fusion polypeptide is reduced or inhibited by an anti-cancer drug, e.g., a DCTN1 inhibitor, an ALK inhibitor. In one embodiment, at least one biological activity of the DCTN1-ALK fusion polypeptide is reduced or inhibited by an ALK inhibitor. In one embodiment, at least one biological activity of the DCTN1-ALK fusion polypeptide is reduced or inhibited by a DCTN1 inhibitor. In one embodiment, at least one biological activity of the DCTN1-ALK fusion polypeptide is reduced or inhibited by an ALK inhibitor, e.g., TAE-684 (also referred to herein as “NVP-TAE694”), PF02341066 (also referred to herein as “crizotinib” or “1066”), AF-802, LDK-378, ASP-3026, CEP-37440, CEP-28122, CEP-108050 and AP26113. Additional examples of ALK kinase inhibitors are described in examples 3-39 of WO 2005016894 by Garcia-Echeverria C, et al.

In yet other embodiments, the DCTN1-ALK fusion polypeptide is encoded by a nucleic acid molecule described herein. In one embodiment, the DCTN1-ALK fusion polypeptide is encoded by an in-frame fusion of intron 26 of DCTN1 with intron 19 of ALK (e.g., a sequence on chromosome 2 and a sequence on chromosome 2. In another embodiment, the DCTN1-ALK fusion polypeptide includes an amino acid sequence encoded by a nucleotide sequence comprising a fusion junction between the DCTN1 transcript and the ALK transcript.

In certain embodiments, the DCTN1-ALK fusion polypeptide comprises one or more of encoded exons 1-26 from DCTN1 and one or more of encoded exons 20-29 of ALK. In certain embodiments, the DCTN1-ALK fusion polypeptide comprises at least 1, 2, 3, 4, 5, 6, 7, 8, 9, 10, 11, 12, 13, 14, 15, 16, 17, 18, 19, 20, 21, 22, 23, 24, 25, 26 or more encoded exons of DCTN1 and at least at least 1, 2, 3, 4, 5, 6, 7, 8, 4, 5, 6, 7, 8, 9, 10 or more encoded exons of ALK. In certain embodiments, the DCTN1-ALK fusion polypeptide comprises a fusion of encoded exons 26 from DCTN1 and encoded exon 20 from ALK (or a fragment thereof). In other embodiments, the fusion comprises at least 1, 2, 3, 4, 5, 6, 7, 8, 9, 10, 11, 12, 13, 14, 15, 16, 17, 18, 19, 20, 21, 22, 23, 24, 25, 26 or more encoded exons of DCTN1; and at least 1, 2, 3, 4, 5, 6, 7, 8, 4, 5, 6, 7, 8, 9, 10 or more encoded exons of ALK. In certain embodiments, the DCTN1-ALK fusion polypeptide comprises encoded exons 1-26 from DCTN1 and exons 20-29 of ALK. In certain embodiments, the 5′ DCTN1-3′ ALK fusion polypeptide comprises a fusion junction of the sequence of exons 26 from DCTN1 and the sequence of exon 20 from ALK.

In certain embodiments, the DCTN1-ALK fusion comprises the amino acid sequence corresponding to exons 26 or a fragment thereof from DCTN1, and the amino acid sequence corresponding to exon 20 or a fragment thereof from ALK (e.g., as shown in SEQ ID NO:67 and SEQ ID NO:7). In one embodiment, the DCTN1-ALK fusion comprises at least 5, 10, 15, 20 or more amino acids from exons 26 of DCTN1 (e.g., from the amino acid sequence of DCTN1 preceding the fusion junction with ALK, e.g., of the DCTN11 sequence shown in SEQ ID NO:67), and at least 5, 10, 15, 20 or more amino acids from exon 20 of ALK (e.g., from the amino acid sequence of ALK following the fusion junction with DCTN1, e.g., of the ALK sequence shown in SEQ ID NO:7).

In one embodiment, the DCTN1-ALK fusion includes the full sequence or a fragment of the nucleotide sequence shown in SEQ ID NO:76, or a nucleotide sequence substantially identical thereto. In another embodiment, the DCTN1-ALK fusion polypeptide includes the full sequence or a fragment of the amino acid sequence shown in SEQ ID NO:77, or an amino acid sequence substantially identical thereto.

In one embodiment, the DCTN1-ALK fusion polypeptide includes a tyrosine kinase domain or a functional fragment thereof. In a related aspect, the invention features DCTN1-ALK fusion polypeptide or fragments operatively linked to heterologous polypeptides to form fusion proteins.

In another embodiment, the DCTN1-ALK fusion polypeptide or fragment is a peptide, e.g., an immunogenic peptide or protein. The peptide contains a fusion junction described herein. Such immunogenic peptides or proteins can be used to raise antibodies specific to the fusion protein. In other embodiments, such immunogenic peptides or proteins can be used for vaccine preparation. The vaccine preparation can include other components, e.g., an adjuvant.

In another aspect, the invention features antibody molecules that bind to a DCTN1-ALK fusion polypeptide or fragment described herein. In embodiments, the antibody can distinguish wild type ALK (or DCTN1 from DCTN1-ALK.

### Detection Reagents and Detection of Mutations

In another aspect, the invention features a detection reagent, e.g., a purified or an isolated preparation thereof. Detection reagents can distinguish a nucleic acid, or protein sequence, having a breakpoint, e.g., a DCTN1-ALK breakpoint; from a reference sequence. In one embodiment, the detection reagent detects (e.g., specifically detects) a DCTN1-ALK fusion nucleic acid or a polypeptide, e.g., distinguishes a wild type ALK or another ALK fusion (or DCTN1) from a DCTN1-ALK nucleic acid (e.g., as described herein in SEQ ID NO:66 and SEQ ID NO:7); or a DCTN1-ALK polypeptide (e.g., as described herein in SEQ ID NO:67 and SEQ ID NO:7).

Detection reagents, e.g., nucleic acid-based detection reagents, can be used to identify mutations in a target nucleic acid. e.g., DNA, e.g., genomic DNA or cDNA, or RNA, e.g., in a sample, e.g., a sample of nucleic acid derived from a neoplastic or tumor cell, e.g., a melanocytic neoplasm, melanoma or metastatic cell. Detection reagents, e.g., antibody-based detection reagents, can be used to identify mutations in a target protein, e.g., in a sample, e.g., a sample of protein derived from, or produced by, a neoplastic or tumor cell, e.g., a melanocytic neoplasm, melanoma or metastatic cell.

### Method of Treatment

In a related aspect, a method of inhibiting, reducing, or treating a hyperproliferative disorder, e.g., a neoplasm (including benign, pre-malignant or malignant (e.g., a cancer), in a subject is provided. The method includes administering to the subject a preselected therapeutic agent, e.g., an anti-cancer agent (e.g., a kinase inhibitor), as a single agent, or in combination, in an amount sufficient to reduce, inhibit or treat the activity or expression of DCTN1-ALK (e.g., a DCTN1-ALK fusion described herein), thereby inhibiting, reducing, or treating the hyperproliferative disorder in the subject.

In one embodiment, the subject treated has a DCTN1-ALK fusion; e.g., the subject has a tumor or cancer harboring a DCTN1-ALK fusion. In other embodiments, the subject has been previously identified as having a DCTN1-ALK fusion. In yet other embodiments, the subject has been previously identified as being likely or unlikely to respond to treatment with a protein kinase inhibitor, e.g., a subject that has previously participated in a clinical trial. In other embodiments, the subject has been previously identified as being likely or unlikely to respond to treatment with a protein kinase inhibitor, based on the presence of the DCTN1-ALK fusion. In one embodiment, the subject is a mammal, e.g., a human. In one embodiment, the subject has, or at risk of having a cancer at any stage of disease. In other embodiments, the subject is a patient, e.g., a cancer patient.

In certain embodiments, the neoplasm or neoplastic cell is a benign, pre-malignant, malignant (cancer) or metastasis. In certain embodiments, the cancer is a solid tumor, a soft tissue tumor, or a metastatic lesion.

In one embodiment, the neoplasm or neoplastic cell is a benign, pre-malignant, malignant (cancer) or metastasis. In certain embodiments, the cancer is a solid tumor, a soft tissue tumor, or a metastatic lesion. In one embodiment, the cancer is a carcinoma. In one embodiment, the cancer is an adenocarcinoma. In an embodiment, the cancer is a lung adenocarcinoma. In one embodiment, the cancer is chosen from a lung cancer, a pancreatic cancer, melanoma, a colorectal cancer, an esophageal-gastric cancer, a thyroid cancer, or an adenocarcinoma. In one embodiment, the cancer is a lymphoma. In one embodiment, the cancer is an anaplastic large cell lymphoma. In one embodiment, the cancer is an inflammatory myofibrotic tumor. In one embodiment, the cancer is a lung adenocarcinoma. In other embodiment, the lung cancer is chosen from one or more of the following: non-small cell lung cancer (NSCLC), small cell lung cancer SCLC), squamous cell carcinoma SCC), adenocarcinoma of the lung, bronchogenic carcinoma, or a combination thereof. In one embodiment, the lung cancer is NSCLC or SCC.

In one embodiment, the anti-cancer agent is an ALK inhibitor. In one embodiment, the anti-cancer agent is a DCTN1 inhibitor. In one embodiment, the anti-cancer agent is a kinase inhibitor. In one embodiment, the anti-cancer agent is an ALK inhibitor, e.g., TAE-684 (also referred to herein as “NVP-TAE694”), PF02341066 (also referred to herein as “crizotinib” or “1066”), AF-802, LDK-378, ASP-3026, CEP-37440, CEP-28122, CEP-108050 and AP26113. Additional examples of ALK kinase inhibitors are described in examples 3-39 of WO 2005016894 by Garcia-Echeverria C, et al.

### LSM14A-BRAF Fusions

In one embodiment, a fusion includes an in-frame fusion of an exon of SCD6 Homolog A (S. cerevisiae), or RNA-Associated Protein 55A (LSM14A), e.g., one more exons of LSM14A (e.g., one or more of exons 1-9 of LSM14A) or a fragment thereof, and an exon of v-raf murine sarcoma viral oncogene homolog B1 (BRAF), e.g., one or more exons of a BRAF (e.g., one or more of exons 9-18 of BRAF) or a fragment thereof. For example, the LSM14A-BRAF fusion can include an in-frame fusion within an intron of LSM14A (e.g., intron 9) or a fragment thereof, with an intron of BRAF (e.g., intron 8) or a fragment thereof. In one embodiment, the fusion of the LSM14A-BRAF fusion comprises the nucleotide sequence of: chromosome 19 at one or more of a nucleotide (plus or minus 10, 20, 30, 50, 60, 70, 80, 100 or more nucleotides) and chromosome 7 at one or more of a nucleotide (plus or minus 10, 20, 30, 50, 60, 70, 80, 100 or more nucleotides). In one embodiment, the LSM14A-BRAF fusion is a translocation, e.g., a translocation of a portion of chromosome 19 and a portion of chromosome 7.

In certain embodiments, the LSM14A-BRAF fusion is in a 5′-LSM14A to 3′-BRAF configuration (also referred to herein as “5′-LSM14A-BRAF-3′).” The term “fusion” or “fusion molecule” can refer to a polypeptide or a nucleic acid fusion, depending on the context. It may include a full-length sequence of a fusion or a fragment thereof, e.g., a fusion junction (e.g., a fragment including a portion of LSM14A and a portion of BRAF, e.g., a portion of the LSM14A-BRAF fusion described herein). In one embodiment, the LSM14A-BRAF fusion polypeptide includes a fragment of the amino acid sequence shown in SEQ ID NO:69 and a fragment of the amino acid sequence shown in SEQ ID NO:2, or an amino acid sequence substantially identical thereto. In another embodiment, the LSM14A-BRAF fusion nucleic acid includes a fragment of the nucleotide sequence shown in SEQ ID NO:68 and a fragment of the nucleotide sequence shown in SEQ ID NO: 1, or a nucleotide sequence substantially identical thereto. In one embodiment, the LSM14A-BRAF fusion polypeptide comprises sufficient LSM14A and sufficient BRAF sequence such that the 5′ LSM14A-3′ BRAF fusion has kinase activity, e.g., has elevated activity, e.g., tyrosine kinase activity. In any event, the fusion causes activation in the cells harboring it of oncogenic signaling pathways.

In certain embodiments, the LSM14A-BRAF fusion comprises one or more (or all of) exons 1-9 from LSM14A and one or more (or all of) exons 9-18 of BRAF (e.g., one or more of the exons shown in SEQ ID NO:68 and SEQ ID NO: 1. In another embodiment, the LSM14A-BRAF fusion comprises one or more (or all of) exons 1-9 of LSM14A and one or more (or all of) exons 9-18 of BRAF. In certain embodiments, the LSM14A-BRAF fusion comprises at least 1, 2, 3, 4, 5, 6, 7, 8, 9 or more exons (or encoded exons) from LSM14A and at least 1, 2, 3, 4, 5, 6, 7, 8, 9, 10 or more exons (or encoded exons) from BRAF (e.g., from the LSM14A and BRAF sequences shown in SEQ ID NO:68 and SEQ ID NO:69 and SEQ ID NO: 1 and SEQ ID NO:2.

In certain embodiments, the LSM14A-BRAF fusion comprises exons 1-9 or a fragment thereof from LSM14A, and exons 9-18 or a fragment thereof from BRAF (e.g., as shown in SEQ ID NO:68 and SEQ ID NO:1). In one embodiment, the LSM14A-BRAF fusion comprises at least 5, 10, 15, 20, 30, 40, 50 or more amino acids from exons 1-9 of LSM14A (e.g., from the amino acid sequence of LSM14A as shown in SEQ ID NO:69 (e.g., from the amino acid sequence of LSM14A preceding the fusion junction with BRAF, and at least 5, 10, 15, 20, 30, 40, 50 or more amino acids from exons 9-18 of BRAF (e.g., from the amino acid sequence of BRAF as shown in SEQ ID NO:2). In another embodiment, the LSM14A-BRAF fusion comprises at least 6, 12, 15, 20, 25, 50, 75, 100) or more nucleotides from exons 1-9 of LSM14A (e.g., from the nucleotide sequence of LSM14A as shown in SEQ ID NO:68 (e.g., from the nucleotide sequence of LSM14A preceding the fusion junction with BRAF); and at least 6, 12, 15, 20, 25, 50, 75, 100 or more nucleotides from exons 9-18 of BRAF (e.g., from the nucleotide sequence of BRAF as shown in SEQ ID NO: 1).

### LSM14A-BRAF Nucleic Acid Molecules

In one aspect, the invention features a nucleic acid molecule (e.g., an isolated or purified) nucleic acid molecule that includes a fragment of a LSM14A gene and a fragment of a BRAF gene. In one embodiment, the nucleotide sequence encodes a LSM14A-BRAF fusion polypeptide that includes a tyrosine kinase domain or a functional fragment thereof. In another embodiment, the nucleotide sequence encodes a fragment of the BRAF polypeptide including the amino acid sequence of SEQ ID NO:2 or a fragment thereof, or a sequence substantially identical thereto. In other embodiments, the nucleic acid molecule includes a fragment of the LSM14A gene encoding the amino acid sequence of SEQ ID NO:69 or a fragment thereof, or a sequence substantially identical thereto. In yet other embodiments, the nucleic acid molecule includes a nucleotide sequence encoding the amino acid sequence shown in SEQ ID NO:69, or a fragment thereof, and the amino acid sequence shown in SEQ ID NO:2 or a fragment thereof, or a sequence substantially identical thereto.

In one embodiment, the nucleic acid molecule includes a fusion. e.g., an in-frame fusion, between an intron of LSM14A (e.g., intron 9, or a fragment thereof), and an intron of BRAF (e.g., intron 8, or a fragment thereof). The LSM14A-BRAF fusion can comprise a fusion of the nucleotide sequence of: chromosome 19 at one or more of a nucleotide (plus or minus 10, 20, 30, 50, 60, 70, 80, 100 nucleotides) and chromosome 7 at one or more of a nucleotide (plus or minus 10, 20, 30, 50, 60, 70, 80, 100 nucleotides), or a fragment thereof. In another embodiment, the LSM14A-BRAF fusion comprises a fusion of the nucleotide sequence of: chromosome 19 at one or more of a nucleotide (plus or minus 10, 20, 30, 50, 60, 70, 80, 100 nucleotides) and chromosome 7 at one or more of a nucleotide (plus or minus 10, 20, 30, 50, 60, 70, 80, 100 nucleotides), or a fragment thereof.

In another embodiment, the LSM14A-BRAF fusion comprises a nucleotide sequence (e.g., a fragment of a nucleotide sequence) shown in SEQ ID NO:68 and a nucleotide sequence (e.g., a fragment of a nucleotide sequence) shown in SEQ ID NO:1, or a fragment of the fusion. In one embodiment, the LSM14A-BRAF fusion comprises a nucleotide sequence substantially identical to the nucleotide sequence (e.g., a fragment of a nucleotide sequence) shown in SEQ ID NO:68 and the nucleotide sequence (e.g., a fragment of a nucleotide sequence) shown SEQ ID NO: 1, or a fragment of the fusion. In one embodiment, the LSM14A-BRAF fusion comprises a nucleotide sequence at least 70%, at least 75%, at least 80%, at least 85%, at least 90%, at least 95%, at least 99%, at least 99.5 or greater, identical to the nucleotide sequence (e.g., a fragment of a nucleotide sequence) shown in SEQ ID NO:68 and to the nucleotide sequence (e.g., a fragment of a nucleotide sequence) shown in SEQ ID NO:1. In one embodiment, the LSM14A-BRAF fusion comprises a nucleotide sequence containing at least 25, 50, 100, 150, 200, 500, 1000, 1500, 2000, 2500, 3000, or more nucleotides of the nucleotide sequence shown in SEQ ID NO:68 and a nucleotide sequence containing at least 25, 50, 100, 150, 200, 500, 1000, 1500, 2000, 2500, 3000, or more nucleotides of the nucleotide sequence shown in SEQ ID NO:1. In one embodiment, the LSM14A-BRAF fusion comprises a nucleotide sequence containing at least 25, 50, 100, 150, 200, 500, 1000, 1500, 2000, 2500, 3000, or more contiguous nucleotides of the nucleotide sequence shown in SEQ ID NO:68 and a nucleotide sequence containing at least 25, 50, 100, 150, 200, 500, 1000, 1500, 2000, 2500, 3000, or more nucleotides of the nucleotide sequence shown in SEQ ID NO:1.

In another embodiment, the nucleic acid molecule includes a fusion, e.g., an in-frame fusion, of at least 1, 2, 3, 4, 5, 6, 7, 8, 9 or more exons of LSM14A or a fragment thereof (e.g., one or more of exons 1-9 of LSM14A or a fragment thereof), and at least 1, 2, 3, 4, 5, 6, 7, 8, 9, 10 or more exons of BRAF or a fragment thereof (e.g., one or more of exons 9-18 of BRAF or a fragment thereof). In yet other embodiments, the nucleic acid molecule includes a fragment the nucleotide sequence shown in SEQ ID NO:68 and a fragment of the nucleotide sequence shown in SEQ ID NO: 1 or a fragment of the fusion, or a sequence substantially identical thereto.

In one embodiment, the nucleic acid molecule is complementary to at least a portion of a nucleotide sequence disclosed herein, e.g., is capable of hybridizing under a stringency condition described herein to SEQ ID NO:68 and/or SEQ ID NO: 1, or a fragment thereof. In yet another embodiment, the nucleic acid molecule hybridizes to a nucleotide sequence that is complementary to at least a portion of a nucleotide sequence disclosed herein, e.g., is capable of hybridizing under a stringency condition to a nucleotide sequence complementary to SEQ ID NO:68 and/or SEQ ID NO: 1, or a fragment thereof. The nucleotide sequence of a cDNA encoding an exemplary 5′ LSM14A-3′ BRAF fusion is shown in at least exon 9 (e.g., exons 1-9) of SEQ ID NO:68 and at least exon 9 (e.g., exons 9-18) of SEQ ID NO: 1, and the predicted amino acid sequence is shown in the corresponding encoded exons of SEQ ID NO:69 and the corresponding encoded exons of SEQ ID NO:2, respectively.

In an embodiment the LSM14A-BRAF nucleic acid molecule comprises sufficient LSM14A and sufficient BRAF sequence such that the encoded 5′ LSM14A-3′ BRAF fusion has kinase activity, e.g., has elevated activity. In certain embodiments, the 5′ LSM14A-3′ BRAF fusion comprises exons 1-9 from LSM14A and exons 9-18 from BRAF. In certain embodiments, the LSM14A-BRAF fusion comprises at least 1, 2, 3, 4, 5, 6, 7, 8, 9 or more exons from LSM14A and at least at least 1, 2, 3, 4, 5, 6, 7, 8, 9, 10 or more exons of BRAF. In certain embodiments, the LSM14A-BRAF fusion comprises a fusion of exon 9 from LSM14A and exon 9 from BRAF. In another embodiment, the LSM14A-BRAF fusion comprises 1, 2, 3, 4, 5, 6, 7, 8, 9 or more exons of LSM14A; and at least 1, 2, 3, 4, 5, 6, 7, 8, 9, 10 or more exons of BRAF.

In one embodiment, the nucleic acid molecule includes a nucleotide sequence that has an in-frame fusion of intron 9 of LSM14A (e.g., NM_{—}001114093) with intron 8 of BRAF (e.g., NM_{—}004333). In another embodiment, the nucleic acid molecule includes a nucleotide sequence that includes a breakpoint. For example, the nucleic acid molecule includes a nucleotide sequence that includes the fusion junction between the LSM14A gene and the BRAF gene, e.g., the breakpoint between intron 9 of LSM14A and intron 8 of BRAF. In other embodiments, the nucleic acid molecules includes a nucleotide sequence of one or more of nucleotide of chromosome 19 coupled to (e.g., directly or indirectly juxtaposed to) one or more of nucleotide of chromosome 7. In one embodiment, the nucleic acid molecule includes the nucleotide sequence of: chromosome 19 at one or more of a nucleotide plus or minus 10, 20, 30, 40 50, 60, 80, 100, 150 nucleotides and chromosome 7 at one or more of a nucleotide plus or minus 10, 20, 30, 40 50, 60, 80, 100, 150 nucleotides (corresponding to the breakpoint of a LSM14A-BRAF fusion), or a fragment thereof, or a sequence substantially identical thereto. In one embodiment, the nucleic acid molecule is complementary to at least a portion of a nucleotide sequence disclosed herein, e.g., is capable of hybridizing under a stringency condition described herein to SEQ ID NO:68 and/or SEQ ID NO: 1 or a fragment thereof. In yet other embodiment, the nucleic acid molecule hybridizes to a nucleotide sequence that is complementary to at least a portion of a nucleotide sequence disclosed herein, e.g., is capable of hybridizing under a stringency condition described herein to a nucleotide sequence complementary to SEQ ID NO:68 or SEQ ID NO:1 or a fragment thereof.

In another embodiment, the LSM14A-BRAF fusion nucleic acid comprises at least 6, 12, 15, 20, 25, 50, 75, 100 or more nucleotides from exon 9 of LSM4A (e.g., from the nucleotide sequence of LSM14A preceding the fusion junction with BRAF, e.g., of the LSM14A sequence shown in SEQ ID NO:68), and at least 6, 12, 15, 20, 25, 50, 75, 100 or more nucleotides from exon 9 of BRAF (e.g., from the nucleotide sequence of BRAF following the fusion junction with LSM14A, e.g., of the BRAF sequence shown in SEQ ID NO: 1).

In other embodiments, the nucleic acid molecule includes a nucleotide sequence encoding a LSM14A-BRAF fusion polypeptide that includes a fragment of a LSM14A gene and a fragment of a BRAF gene. In one embodiment, the nucleotide sequence encodes a LSM14A-BRAF fusion polypeptide that includes e.g., a tyrosine kinase domain or a functional fragment thereof. In yet other embodiments, the nucleic acid molecule includes a nucleotide sequence encoding the amino acid sequence (e.g., a fragment of the amino acid sequence) shown in SEQ ID NO:69 and a nucleotide sequence encoding the amino acid sequence (e.g., a fragment of the amino acid sequence) shown in SEQ ID NO:2, or a fragment of the fusion, or a sequence substantially identical thereto. In one embodiment, the encoded LSM14A-BRAF fusion polypeptide includes a tyrosine kinase domain or a functional fragment thereof.

In a related aspect, the invention features nucleic acid constructs that include the LSM14A-BRAF nucleic acid molecules described herein. In certain embodiments, the nucleic acid molecules are operatively linked to a native or a heterologous regulatory sequence. Also included are vectors and host cells that include the LSM14A-BRAF nucleic acid molecules described herein, e.g., vectors and host cells suitable for producing the nucleic acid molecules and polypeptides described herein.

In a related aspect, methods of producing the nucleic acid molecules and polypeptides described herein are also described.

In another aspect, the invention features nucleic acid molecules that reduce or inhibit the expression of a nucleic acid molecule that encodes a LSM14A-BRAF fusion described herein. Examples of such nucleic acid molecules include, for example, antisense molecules, ribozymes, RNAi, triple helix molecules that hybridize to a nucleic acid encoding LSM14A-BRAF, or a transcription regulatory region of LSM14A-BRAF, and blocks or reduces mRNA expression of LSM14A-BRAF.

### Nucleic Acid Detection and Capturing Reagents

The invention also features a nucleic acid molecule, e.g., nucleic acid fragment, suitable as probe, primer, bait or library member that includes, flanks, or hybridizes to the LSM14A-BRAF fusions described herein. Such nucleic acid molecules are useful for identifying, or are otherwise based on, the LSM14A-BRAF fusions described herein. In certain embodiments, the probe, primer or bait molecule is an oligonucleotide that allows capture, detection or isolation of a LSM14A-BRAF fusion nucleic acid molecule described herein. The oligonucleotide can comprise a nucleotide sequence substantially complementary to a fragment of the LSM14A-BRAF fusion nucleic acid molecules described herein. The sequence identity between the nucleic acid fragment. e.g., the oligonucleotide, and the target LSM14A-BRAF sequence need not be exact, so long as the sequences are sufficiently complementary to allow the capture, detection or isolation of the target sequence. In one embodiment, the nucleic acid fragment is a probe or primer that includes an oligonucleotide between about 5 and 25, e.g., between 10 and 20, or 10 and 15 nucleotides in length. In other embodiments, the nucleic acid fragment is a bait that includes an oligonucleotide between about 100 to 300 nucleotides, 130 and 230 nucleotides, or 150 and 200 nucleotides, in length.

In one embodiment, the nucleic acid fragment can be used to identify or capture, e.g., by hybridization, a LSM14A-BRAF fusion. For example, the nucleic acid fragment can be a probe, a primer, or a bait, for use in identifying or capturing, e.g., by hybridization, a LSM14A-BRAF fusion described herein. In one embodiment, the nucleic acid fragment can be useful for identifying or capturing a LSM14A-BRAF breakpoint, e.g., the nucleotide sequence of: chromosome 19 at nucleotide plus or minus 10, 20, 30, 40, 50, 60, 80, 100, 150 nucleotides and chromosome 7 at nucleotide plus or minus 10, 20, 30, 40 50, 60, 80, 100, 150 nucleotides.

In one embodiment, the nucleic acid fragment hybridizes to a nucleotide sequence within a chromosomal rearrangement that creates an in-frame fusion of intron 9 of LSM14A with intron 8 of BRAF. In one embodiment, the nucleic acid fragment hybridizes to a nucleotide sequence in the region In other embodiments, the nucleic acid molecules includes a nucleotide sequence in the region of a nucleotide sequence of chromosome 19 coupled to (e.g., juxtaposed to) nucleotides in the region of a nucleotide sequence of chromosome 17. In one embodiment, the nucleic acid fragment hybridizes to a nucleotide sequence that includes a breakpoint, e.g., the nucleotide sequence of: chromosome 19 at nucleotide plus or minus 10, 20, 30, 40 50, 60, 80, 100, 150 or more nucleotides and chromosome 7 at nucleotide plus or minus 10, 20, 30, 40 50, 60, 80, 100, 150 or more nucleotides. For example, the nucleic acid fragment can hybridize to a nucleotide sequence that includes the fusion junction between the LSM14A gene and the BRAF gene, e.g., a nucleotide sequence that includes a portion of a nucleotide sequence within intron 9 of a LSM14A gene and intron 8 of a BRAF gene.

In another embodiment, the nucleic acid fragment hybridizes to a nucleotide sequence that comprises at least 6, 12, 15, 20, 25, 50, 75, 100, 150 or more nucleotides from exon 9 of LSM14A (e.g., from the nucleotide sequence of LSM14A preceding the fusion junction with BRAF, e.g., of the LSM14A sequence shown in SEQ ID NO:68), and at least 6, 12, 15, 20, 25, 50, 75, 100, 150 or more nucleotides from exon 9 of BRAF (e.g., from the nucleotide sequence of BRAF following the fusion junction with LSM14A, e.g., of the BRAF sequence shown in SEQ ID NO: 1).

The probes or primers described herein can be used, for example, for FISH detection or PCR amplification. In one exemplary embodiment where detection is based on PCR, amplification of the LSM14A-BRAF fusion junction fusion junction can be performed using a primer or a primer pair, e.g., for amplifying a sequence flanking the fusion junctions described herein, e.g., the mutations or the junction of a chromosomal rearrangement described herein, e.g., LSM14A-BRAF.

In one embodiment, a pair of isolated oligonucleotide primers can amplify a region containing or adjacent to a position in the LSM14A-BRAF fusion. For example, forward primers can be designed to hybridize to a nucleotide sequence within LSM14A genomic or mRNA sequence (e.g., a nucleotide sequence within exon 9 of LSM14A of SEQ ID NO:68, and the reverse primers can be designed to hybridize to a nucleotide sequence of BRAF (e.g., a nucleotide sequence within exon 9 of BRAF, of SEQ ID NO:1.

In another embodiment, the nucleic acid fragments can be used to identify, e.g., by hybridization, a LSM14A-BRAF fusion. In one embodiment, the nucleic acid fragment hybridizes to a nucleotide sequence that includes a fusion junction between the LSM14A transcript and the BRAF transcript.

In other embodiments, the nucleic acid fragment includes a bait that comprises a nucleotide sequence that hybridizes to a LSM14A-BRAF fusion nucleic acid molecule described herein, and thereby allows the capture or isolation said nucleic acid molecule. In one embodiment, a bait is suitable for solution phase hybridization. In other embodiments, a bait includes a binding entity, e.g., an affinity tag, that allows capture and separation, e.g., by binding to a binding entity, of a hybrid formed by a bait and a nucleic acid hybridized to the bait.

In other embodiments, the nucleic acid fragment includes a library member comprising a LSM14A-BRAF nucleic acid molecule described herein. In one embodiment, the library member includes a rearrangement that results in a LSM14A-BRAF fusion described herein.

The nucleic acid fragment can be detectably labeled with, e.g., a radiolabel, a fluorescent label, a bioluminescent label, a chemiluminescent label, an enzyme label, a binding pair label, or can include an affinity tag; a tag, or identifier (e.g., an adaptor, barcode or other sequence identifier).

### LSM14A-BRAF Fusion Polypeptides

In another embodiment, the LSM14A-BRAF fusion comprises an amino acid sequence (e.g., a fragment of the amino acid sequence) shown in SEQ ID NO:69 and an amino acid sequence (e.g., a fragment of the amino acid sequence) shown in SEQ ID NO:2, or a fragment of the fusion. In one embodiment, the LSM14A-BRAF fusion comprises an amino acid sequence substantially identical to the amino acid sequence (e.g., a fragment of the amino acid sequence) shown in SEQ ID NO:69 and the amino acid sequence (e.g., a fragment of the amino acid sequence) shown in SEQ ID NO:2, or a fragment thereof. In one embodiment, the LSM14A-BRAF fusion comprises an amino acid sequence at least 70%, at least 75%, at least 80%, at least 85%, at least 90%, at least 95%, at least 99%, at least 99.5 or greater, identical to the amino acid sequence (e.g., a fragment of the amino acid sequence) shown in 142 SEQ ID NO:69 and the amino acid sequence (e.g., a fragment of the amino acid sequence) shown in SEQ ID NO:2. In one embodiment, the LSM14A-BRAF fusion comprises a sequence containing at least 10, 20, 50, 100, 500, 600, 700, 800, 900, 1000, or more amino acids of the amino acid sequence shown in SEQ ID NO:69 and SEQ ID NO:2. In one embodiment, the LSM14A-BRAF fusion comprises an amino acid sequence containing at least 5, 10, 20, 50, 100, 500, 600, 700, 800, 900, 1000, or more contiguous amino acids of the amino acid sequence shown in SEQ ID NO:69 and at least 5, 10, 20, 50, 100, 500, 600, 700, 800, 900, 1000, or more contiguous amino acids of the amino acid sequence shown in SEQ ID NO:2. In one embodiment, the 5′ LSM14A-3′ BRAF fusion polypeptide includes a receptor tyrosine kinase domain or a functional fragment thereof. In an embodiment, the 5′LSM14A-3′BRAF fusion polypeptide comprises sufficient BRAF and sufficient LSM14A sequence such that it has kinase activity, e.g., has elevated activity. In any event, the fusion causes activation in the cells harboring it of oncogenic signaling pathways.

In another aspect, the invention features a LSM14A-BRAF fusion polypeptide (e.g., a purified LSM14A-BRAF fusion polypeptide), a biologically active or antigenic fragment thereof, as well as reagents (e.g., antibody molecules that bind to a LSM14A-BRAF fusion polypeptide), methods for modulating a LSM14A-BRAF polypeptide activity and detection of a LSM14A-BRAF polypeptide.

In one embodiment, the LSM14A-BRAF fusion polypeptide has at least one biological activity. In one embodiment, at least one biological activity of the LSM14A-BRAF fusion polypeptide is reduced or inhibited by an anti-cancer drug, e.g., a LSM14A inhibitor, a BRAF inhibitor. In one embodiment, at least one biological activity of the LSM14A-BRAF fusion polypeptide is reduced or inhibited by a BRAF inhibitor. In one embodiment, at least one biological activity of the LSM14A-BRAF fusion polypeptide is reduced or inhibited by a LSM14A inhibitor. In one embodiment, at least one biological activity of the LSM14A-BRAF fusion polypeptide is reduced or inhibited by a BRAF inhibitor, e.g., vemurafenib (also known as RG7204; or PLX4032; or Zelboraf); GDC-0879; PLX-4702; AZ628; dabrafenib (GSK2118346A); or Sorafenib Tosylate.

In yet other embodiments, the LSM14A-BRAF fusion polypeptide is encoded by a nucleic acid molecule described herein. In one embodiment, the LSM14A-BRAF fusion polypeptide is encoded by an in-frame fusion of intron 9 of LSM14A with intron 8 of BRAF (e.g., a sequence on chromosome 19 and a sequence on chromosome 7). In another embodiment, the LSM14A-BRAF fusion polypeptide includes an amino acid sequence encoded by a nucleotide sequence comprising a fusion junction between the LSM14A transcript and the BRAF transcript.

In certain embodiments, the LSM14A-BRAF fusion polypeptide comprises one or more of encoded exons 1-9 from LSM14A and one or more of encoded exons 9-18 of BRAF. In certain embodiments, the LSM14A-BRAF fusion polypeptide comprises at least 1, 2, 3, 4, 5, 6, 7, 8, 9 or more encoded exons of LSM14A and at least at least 1, 2, 3, 4, 5, 6, 7, 8, 9, 10 or more encoded exons of BRAF. In certain embodiments, the LSM14A-BRAF fusion polypeptide comprises a fusion of encoded exon 9 from LSM14A and encoded exon 9 from BRAF (or a fragment thereof). In other embodiments, the fusion comprises at least 1, 2, 3, 4, 5, 6, 7, 8, 9 or more encoded exons of LSM14A; and at least 1, 2, 3, 4, 5, 6, 7, 8, 9, 10 or more encoded exons of BRAF. In certain embodiments, the LSM14A-BRAF fusion polypeptide comprises encoded exons 1-9 from LSM14A and exons 9-18 of BRAF. In certain embodiments, the 5′ LSM14A-3′ BRAF fusion polypeptide comprises a fusion junction of the sequence of exon 9 from LSM14A and the sequence of exon 9 from BRAF.

In certain embodiments, the LSM14A-BRAF fusion comprises the amino acid sequence corresponding to exon 9 or a fragment thereof from LSM14A, and the amino acid sequence corresponding to exon 9 or a fragment thereof from BRAF (e.g., as shown in SEQ ID NO:69 and SEQ ID NO:2). In one embodiment, the LSM14A-BRAF fusion comprises at least 5, 10, 15, 20 or more amino acids from exon 9 of LSM14A (e.g., from the amino acid sequence of LSM14A preceding the fusion junction with BRAF, e.g., of the LSM14A sequence shown in SEQ ID NO:69), and at least 5, 10, 15, 20 or more amino acids from exon 9 of BRAF (e.g., from the amino acid sequence of BRAF following the fusion junction with LSM14A, e.g., of the BRAF sequence shown in SEQ ID NO:2).

In one embodiment, the LSM14A-BRAF fusion includes the full sequence or a fragment of the nucleotide sequence shown in SEQ ID NO:78, or a nucleotide sequence substantially identical thereto. In another embodiment, the LSM14A-BRAF fusion polypeptide includes the full sequence or a fragment of the amino acid sequence shown in SEQ ID NO:79, or an amino acid sequence substantially identical thereto.

In one embodiment, the LSM14A-BRAF fusion polypeptide includes a tyrosine kinase domain or a functional fragment thereof. In a related aspect, the invention features LSM14A-BRAF fusion polypeptide or fragments operatively linked to heterologous polypeptides to form fusion proteins.

In another embodiment, the LSM14A-BRAF fusion polypeptide or fragment is a peptide, e.g., an immunogenic peptide or protein containing a fusion junction described herein. Such immunogenic peptides or proteins can be used to raise antibodies specific to the fusion protein. In other embodiments, such immunogenic peptides or proteins can be used for vaccine preparation. The vaccine preparation can include other components, e.g., an adjuvant.

In another aspect, the invention features antibody molecules that bind to a LSM14A-BRAF fusion polypeptide or fragment described herein. In embodiments, the antibody can distinguish wild type BRAF (or LSM14A) from LSM14A-BRAF.

### Detection Reagents and Detection of Mutations

In another aspect, the invention features a detection reagent, e.g., a purified or an isolated preparation thereof. Detection reagents can distinguish a nucleic acid, or protein sequence, having a breakpoint, e.g., a LSM14A-BRAF breakpoint; from a reference sequence. In one embodiment, the detection reagent detects (e.g., specifically detects) a LSM14A-BRAF fusion nucleic acid or a polypeptide, e.g., distinguishes a wild type BRAF or another BRAF fusion (or LSM14A) from a LSM14A-BRAF nucleic acid (e.g., as described herein in SEQ ID NO:68 and SEQ ID NO:1); or a LSM14A-BRAF polypeptide (e.g., as described herein in SEQ ID NO:69 and SEQ ID NO:2).

Detection reagents, e.g., nucleic acid-based detection reagents, can be used to identify mutations in a target nucleic acid, e.g., DNA, e.g., genomic DNA or cDNA, or RNA, e.g., in a sample, e.g., a sample of nucleic acid derived from a neoplastic or tumor cell, e.g., a melanocytic neoplasm, melanoma or metastatic cell. Detection reagents, e.g., antibody-based detection reagents, can be used to identify mutations in a target protein. e.g., in a sample, e.g., a sample of protein derived from, or produced by, a neoplastic or tumor cell, e.g., a melanocytic neoplasm, melanoma or metastatic cell.

### Method of Treatment

In a related aspect, a method of inhibiting, reducing, or treating a hyperproliferative disorder, e.g., a neoplasm (including benign, pre-malignant or malignant (e.g., a cancer), in a subject is provided. The method includes administering to the subject a preselected therapeutic agent, e.g., an anti-cancer agent (e.g., a kinase inhibitor), as a single agent, or in combination, in an amount sufficient to reduce, inhibit or treat the activity or expression of LSM14A-BRAF (e.g., a LSM14A-BRAF fusion described herein), thereby inhibiting, reducing, or treating the hyperproliferative disorder in the subject.

In one embodiment, the subject treated has a LSM14A-BRAF fusion; e.g., the subject has a tumor or cancer harboring a LSM14A-BRAF fusion. In other embodiments, the subject has been previously identified as having a LSM14A-BRAF fusion. In yet other embodiments, the subject has been previously identified as being likely or unlikely to respond to treatment with a protein kinase inhibitor. e.g., a subject that has previously participated in a clinical trial. In other embodiments, the subject has been previously identified as being likely or unlikely to respond to treatment with a protein kinase inhibitor, based on the presence of the LSM14A-BRAF fusion. In one embodiment, the subject is a mammal, e.g., a human. In one embodiment, the subject has, or at risk of having a cancer at any stage of disease. In other embodiments, the subject is a patient, e.g., a cancer patient.

In certain embodiments, the neoplasm or neoplastic cell is a benign, pre-malignant, malignant (cancer) or metastasis. In certain embodiments, the cancer is a solid tumor, a soft tissue tumor, or a metastatic lesion.

In one embodiment, the neoplasm or neoplastic cell is a benign, pre-malignant, malignant (cancer) or metastasis. In certain embodiments, the cancer is a solid tumor, a soft tissue tumor, or a metastatic lesion. In one embodiment, the cancer is a carcinoma. In one embodiment the cancer is a papillary thyroid carcinoma. In one embodiment the cancer is a pilocytic astrocytomas. In one embodiment, the cancer is a melanocytic tumor. In one embodiment, the cancer is an adenocarcinoma. In an embodiment, the cancer is a lung adenocarcinoma. In one embodiment, the cancer is chosen from a lung cancer, a pancreatic cancer, melanoma, a colorectal cancer, an esophageal-gastric cancer, a thyroid cancer, or an adenocarcinoma. In one embodiment, the cancer is a lung adenocarcinoma. In other embodiment, the lung cancer is chosen from one or more of the following: non-small cell lung cancer (NSCLC), small cell lung cancer (SCLC), squamous cell carcinoma (SCC), adenocarcinoma of the lung, bronchogenic carcinoma, or a combination thereof. In one embodiment, the lung cancer is NSCLC or SCC.

In one embodiment, the anti-cancer agent is a BRAF inhibitor. In one embodiment, the anti-cancer agent is a LSM14A inhibitor. In one embodiment, the anti-cancer agent is a kinase inhibitor. In one embodiment, the anti-cancer agent is a BRAF inhibitor, e.g., vemurafenib (also known as RG7204; or PLX4032; or Zelboraf); GDC-0879; PLX-4702; AZ628; dabrafenib (GSK2118346A); or Sorafenib Tosylate.

### LMNA-NTRK1 Fusions

In one embodiment, a fusion includes an in-frame fusion of an exon of Lamin A/C (LMNA), e.g., one more exons of LMNA (e.g., one or more of exons 1-2 LMNA) or a fragment thereof, and an exon of neurotrophic tyrosine kinase receptor type 1 (NTRK1), e.g., one or more exons of NTRK1 (e.g., one or more of exons 9-17 of NTRK1) or a fragment thereof. For example, the LMNA-NTRK1 fusion can include an in-frame fusion within an intron of LMNA (e.g., intron 2) or a fragment thereof, with an intron of NTRK1 (e.g., intron 8) or a fragment thereof. In one embodiment, the fusion of the LMNA-NTRK1 fusion comprises the nucleotide sequence of: chromosome 1 at one or more of a nucleotide (plus or minus 10, 20, 30, 50, 60, 70, 80, 100 or more nucleotides) and chromosome 1 at one or more of a nucleotide (plus or minus 10, 20, 30, 50, 60, 70, 80, 100 or more nucleotides). In one embodiment, the LMNA-NTRK1 fusion is a translocation, e.g., a translocation of a portion of chromosome 1 and a portion of chromosome 1.

In certain embodiments, the LMNA-NTRK1 fusion is in a 5′-LMNA to 3′-NTRK1 configuration (also referred to herein as “5′-LMNA-NTRK1-3′).” The term “fusion” or “fusion molecule” can refer to a polypeptide or a nucleic acid fusion, depending on the context. It may include a full-length sequence of a fusion or a fragment thereof, e.g., a fusion junction (e.g., a fragment including a portion of LMNA and a portion of NTRK1, e.g., a portion of the LMNA-NTRK1 fusion described herein). In one embodiment, the LMNA-NTRK1 fusion polypeptide includes a fragment of the amino acid sequence shown in SEQ ID NO:71 and a fragment of the amino acid sequence shown in SEQ ID NO:4, or an amino acid sequence substantially identical thereto. In another embodiment, the LMNA-NTRK1 fusion nucleic acid includes a fragment of the nucleotide sequence shown in SEQ ID NO:70 and a fragment of the nucleotide sequence shown in SEQ ID NO:3, or a nucleotide sequence substantially identical thereto. In one embodiment, the LMNA-NTRK1 fusion polypeptide comprises sufficient LMNA and sufficient NTRK1 sequence such that the 5′ LMNA-3′ NTRK1 fusion has kinase activity, e.g., has elevated activity, e.g., tyrosine kinase activity. In any event, the fusion causes activation in the cells harboring it of oncogenic signaling pathways.

In certain embodiments, the LMNA-NTRK1 fusion comprises one or more (or all of) exons 1-2 from LMNA and one or more (or all of) exons 9-17 of NTRK1 (e.g., one or more of the exons shown in SEQ ID NO:70 and SEQ ID NO:3). In another embodiment, the LMNA-NTRK fusion comprises one or more (or all of) exons 1-2 of LMNA and one or more (or all of) exons 9-17 of NTRK1. In certain embodiments, the LMNA-NTRK1 fusion comprises at least 1, 2 or more exons (or encoded exons) from LMNA and at least 1, 2, 3, 4, 5, 6, 7, 8, 9 or more exons (or encoded exons) from NTRK1 (e.g., from the LMNA and NTRK1 sequences shown in SEQ ID NO:70 and SEQ ID NO:71 and SEQ ID NO:3 and SEQ ID NO:4.

In certain embodiments, the LMNA-NTRK1 fusion comprises exons 1-2 or a fragment thereof from LMNA, and exons 9-17 or a fragment thereof from NTRK1 (e.g., as shown in SEQ ID NO:70 and SEQ ID NO:3). In one embodiment, the LMNA-NTRK1 fusion comprises at least 5, 10, 15, 20, 30, 40, 50 or more amino acids from exons 1-2 of LMNA (e.g., from the amino acid sequence of LMNA as shown in SEQ ID NO:71 (e.g., from the amino acid sequence of LMNA preceding the fusion junction with NTRK1, and at least 5, 10, 15, 20, 30, 40, 50 or more amino acids from exons 9-17 of NTRK1 (e.g., from the amino acid sequence of NTRK1 as shown SEQ ID NO:4). In another embodiment, the LMNA-NTRK1 fusion comprises at least 6, 12, 15, 20, 25, 50, 75, 100 or more nucleotides from exons 1-2 of LMNA (e.g., from the nucleotide sequence of LMNA as shown in SEQ ID NO:70 (e.g., from the nucleotide sequence of LMNA preceding the fusion junction with NTRK1); and at least 6, 12, 15, 20, 25, 50, 75, 100 or more nucleotides from exons 9-17 of NTRK1 (e.g., from the nucleotide sequence of NTRK1 as shown in SEQ ID NO:3).

### LMNA-NTRK1 Nucleic Acid Molecules

In one aspect, the invention features a nucleic acid molecule (e.g., an isolated or purified) nucleic acid molecule that includes a fragment of a LMNA gene and a fragment of a NTRK1 gene. In one embodiment, the nucleotide sequence encodes a LMNA-NTRK1 fusion polypeptide that includes a tyrosine kinase domain or a functional fragment thereof. In another embodiment, the nucleotide sequence encodes a fragment of the NTRK1 polypeptide including the amino acid sequence of SEQ ID NO:4 or a fragment thereof, or a sequence substantially identical thereto. In other embodiments, the nucleic acid molecule includes a fragment of the LMNA gene encoding the amino acid sequence of SEQ ID NO:71 or a fragment thereof, or a sequence substantially identical thereto. In yet other embodiments, the nucleic acid molecule includes a nucleotide sequence encoding the amino acid sequence shown in SEQ ID NO:71, or a fragment thereof, and the amino acid sequence shown in SEQ ID NO:4 or a fragment thereof, or a sequence substantially identical thereto.

In one embodiment, the nucleic acid molecule includes a fusion, e.g., an in-frame fusion, between an intron of LMNA (e.g., intron 2 or a fragment thereof), and an intron of NTRK1 (e.g., intron 8, or a fragment thereof). The LMNA-NTRK1 fusion can comprise a fusion of the nucleotide sequence of: chromosome 1 at one or more of a nucleotide (plus or minus 10, 20, 30, 50, 60, 70, 80, 100 nucleotides) and chromosome 1 at one or more of a nucleotide (plus or minus 10, 20, 30, 50, 60, 70, 80, 100 nucleotides), or a fragment thereof. In one embodiment, the LMNA-NTRK1 fusion comprises a fusion of the nucleotide sequence of: chromosome 1 at one or more of a nucleotide (plus or minus 10, 20, 30, 50, 60, 70, 80, 100 nucleotides) and chromosome 1 at one or more of a nucleotide (plus or minus 10, 20, 30, 50, 60, 70, 80, 100 nucleotides), or a fragment thereof.

In another embodiment, the LMNA-NTRK1 fusion comprises a nucleotide sequence (e.g., a fragment of a nucleotide sequence) shown in SEQ ID NO:70 and a nucleotide sequence (e.g., a fragment of a nucleotide sequence) shown in SEQ ID NO:3, or a fragment of the fusion. In one embodiment, the LMNA-NTRK1 fusion comprises a nucleotide sequence substantially identical to the nucleotide sequence (e.g., a fragment of a nucleotide sequence) shown in SEQ ID NO:70 and the nucleotide sequence (e.g., a fragment of a nucleotide sequence) shown SEQ ID NO:3, or a fragment of the fusion. In one embodiment, the LMNA-NTRK1 fusion comprises a nucleotide sequence at least 70%, at least 75%, at least 80%, at least 85%, at least 90%, at least 95%, at least 99%, at least 99.5 or greater, identical to the nucleotide sequence (e.g., a fragment of a nucleotide sequence) shown in SEQ ID NO:70 and to the nucleotide sequence (e.g., a fragment of a nucleotide sequence) shown in SEQ ID NO:3. In one embodiment, the LMNA-NTRK1 fusion comprises a nucleotide sequence containing at least 25, 50, 100, 150, 200, 500, 1000, 1500, 2000, 2500, 3000, or more nucleotides of the nucleotide sequence shown in SEQ ID NO:70 and a nucleotide sequence containing at least 25, 50, 100, 150, 200, 500, 1000, 1500, 2000, 2500, 3000, or more nucleotides of the nucleotide sequence shown in SEQ ID NO:3. In one embodiment, the LMNA-NTRK1 fusion comprises a nucleotide sequence containing at least 25, 50, 100, 150, 200, 500, 1000, 1500, 2000, 2500, 3000, or more contiguous nucleotides of the nucleotide sequence shown in 155 SEQ ID NO:70 and a nucleotide sequence containing at least 25, 50, 100, 150, 200, 500, 1000, 1500, 2000, 2500, 3000, or more nucleotides of the nucleotide sequence shown in SEQ ID NO:3.

In another embodiment, the nucleic acid molecule includes a fusion, e.g., an in-frame fusion, of at least 1, 2 or more exons of LMNA or a fragment thereof (e.g., one or more of exons 1-2 of LMNA or a fragment thereof), and at least 1, 2, 3, 4, 5, 6, 7, 8, 9 or more exons of NTRK1 or a fragment thereof (e.g., one or more of exons 9-17 of NTRK1 or a fragment thereof). In yet other embodiments, the nucleic acid molecule includes a fragment the nucleotide sequence shown in SEQ ID NO:70 and a fragment of the nucleotide sequence shown in SEQ ID NO:3 or a fragment of the fusion, or a sequence substantially identical thereto.

In one embodiment, the nucleic acid molecule is complementary to at least a portion of a nucleotide sequence disclosed herein, e.g., is capable of hybridizing under a stringency condition described herein to SEQ ID NO:70 and/or SEQ ID NO:3, or a fragment thereof. In yet another embodiment, the nucleic acid molecule hybridizes to a nucleotide sequence that is complementary to at least a portion of a nucleotide sequence disclosed herein, e.g., is capable of hybridizing under a stringency condition to a nucleotide sequence complementary to SEQ ID NO:70 and/or SEQ ID NO:3, or a fragment thereof. The nucleotide sequence of a cDNA encoding an exemplary 5′ LMNA-3′ NTRK1 fusion is shown in at least exon 2 (e.g., exons 1-2) of SEQ ID NO:70 and at least exon 9 (e.g., exons 9-17) of SEQ ID NO:3, and the predicted amino acid sequence is shown in the corresponding encoded exons of SEQ ID NO:71 and the corresponding encoded exons of SEQ ID NO:4, respectively.

In an embodiment the LMNA-NTRK1 nucleic acid molecule comprises sufficient LMNA and sufficient NTRK1 sequence such that the encoded 5′ LMNA-3′ NTRK1 fusion has kinase activity, e.g., has elevated activity. In certain embodiments, the 5′ LMNA-3′ NTRK1 fusion comprises exons 1-2 from LMNA and exons 9-17 from NTRK1. In certain embodiments, the LMNA-NTRK fusion comprises at least 1, 2 or more exons from LMNA and at least at least 1, 2, 3, 4, 5, 6, 7, 8, 9 or more exons of NTRK1. In certain embodiments, the LMNA-NTRK1 fusion comprises a fusion of exon 2 from LMNA and exon 9 from NTRK1. In another embodiment, the LMNA-NTRK1 fusion comprises 1, 2 or more exons of LMNA; and at least 1, 2, 3, 4, 5, 6, 7, 8, 9 or more exons of NTRK1.

In one embodiment, the nucleic acid molecule includes a nucleotide sequence that has an in-frame fusion of intron 2 of LMNA (e.g., NM_{—}001126113) with intron 8 of NTRK1 (e.g., NM_{—}002529). In another embodiment, the nucleic acid molecule includes a nucleotide sequence that includes a breakpoint. For example, the nucleic acid molecule includes a nucleotide sequence that includes the fusion junction between the LMNA gene and the NTRK1 gene, e.g., the breakpoint between intron 2 or intron 9 or intron 11 or intron 12 of LMNA and intron 2 of NTRK1. In other embodiments, the nucleic acid molecules includes a nucleotide sequence of one or more of nucleotide of chromosome 1 coupled to (e.g., directly or indirectly juxtaposed to) one or more of nucleotide of chromosome 1. In one embodiment, the nucleic acid molecule includes the nucleotide sequence of: chromosome 1 at one or more of a nucleotide plus or minus 10, 20, 30, 40 50, 60, 80, 100, 150 nucleotides and chromosome 1 at one or more of a nucleotide plus or minus 10, 20, 30, 40 50, 60, 80, 100, 150 nucleotides (corresponding to the breakpoint of a LMNA-NTRK fusion), or a fragment thereof, or a sequence substantially identical thereto. In one embodiment, the nucleic acid molecule is complementary to at least a portion of a nucleotide sequence disclosed herein, e.g., is capable of hybridizing under a stringency condition described herein to SEQ ID NO:70 and/or SEQ ID NO:3 or a fragment thereof. In yet other embodiment, the nucleic acid molecule hybridizes to a nucleotide sequence that is complementary to at least a portion of a nucleotide sequence disclosed herein, e.g., is capable of hybridizing under a stringency condition described herein to a nucleotide sequence complementary to SEQ ID NO:70 or SEQ ID NO:3 or a fragment thereof.

In another embodiment, the LMNA-NTRK1 fusion nucleic acid comprises at least 6, 12, 15, 20, 25, 50, 75, 100 or more nucleotides from exon 2 or exon 9 or exon 11 or exon 12 of LMNA (e.g., from the nucleotide sequence of LMNA preceding the fusion junction with NTRK1, e.g., of the LMNA sequence shown in SEQ ID NO:70), and at least 6, 12, 15, 20, 25, 50, 75, 100 or more nucleotides from exon 9 of NTRK1 (e.g., from the nucleotide sequence of NTRK1 following the fusion junction with LMNA, e.g., of the NTRK1 sequence shown in SEQ ID NO:3).

In other embodiments, the nucleic acid molecule includes a nucleotide sequence encoding a LMNA-NTRK1 fusion polypeptide that includes a fragment of a LMNA gene and a fragment of a NTRK1 gene. In one embodiment, the nucleotide sequence encodes a LMNA-NTRK1 fusion polypeptide that includes e.g., a tyrosine kinase domain or a functional fragment thereof. In yet other embodiments, the nucleic acid molecule includes a nucleotide sequence encoding the amino acid sequence (e.g., a fragment of the amino acid sequence) shown in SEQ ID NO:71 and a nucleotide sequence encoding the amino acid sequence (e.g., a fragment of the amino acid sequence) shown in SEQ ID NO:4, or a fragment of the fusion, or a sequence substantially identical thereto. In one embodiment, the encoded LMNA-NTRK11 fusion polypeptide includes a tyrosine kinase domain or a functional fragment thereof.

In a related aspect, the invention features nucleic acid constructs that include the LMNA-NTRK1 nucleic acid molecules described herein. In certain embodiments, the nucleic acid molecules are operatively linked to a native or a heterologous regulatory sequence. Also included are vectors and host cells that include the LMNA-NTRK1 nucleic acid molecules described herein, e.g., vectors and host cells suitable for producing the nucleic acid molecules and polypeptides described herein.

In a related aspect, methods of producing the nucleic acid molecules and polypeptides described herein are also described.

In another aspect, the invention features nucleic acid molecules that reduce or inhibit the expression of a nucleic acid molecule that encodes a LMNA-NTRK1 fusion described herein. Examples of such nucleic acid molecules include, for example, antisense molecules, ribozymes, RNAi, triple helix molecules that hybridize to a nucleic acid encoding LMNA-NTRK1, or a transcription regulatory region of LMNA-NTRK1, and blocks or reduces mRNA expression of LMNA-NTRK1.

### Nucleic Acid Detection and Capturing Reagents

The invention also features a nucleic acid molecule, e.g., nucleic acid fragment, suitable as probe, primer, bait or library member that includes, flanks, or hybridizes to the LMNA-NTRK1 fusions described herein. Such nucleic acid molecules are useful for identifying, or are otherwise based on, the LMNA-NTRK1 fusions described herein. In certain embodiments, the probe, primer or bait molecule is an oligonucleotide that allows capture, detection or isolation of a LMNA-NTRK1 fusion nucleic acid molecule described herein. The oligonucleotide can comprise a nucleotide sequence substantially complementary to a fragment of the LMNA-NTRK1 fusion nucleic acid molecules described herein. The sequence identity between the nucleic acid fragment, e.g., the oligonucleotide, and the target LMNA-NTRK1 sequence need not be exact, so long as the sequences are sufficiently complementary to allow the capture, detection or isolation of the target sequence. In one embodiment, the nucleic acid fragment is a probe or primer that includes an oligonucleotide between about 5 and 25, e.g., between 10 and 20, or 10 and 15 nucleotides in length. In other embodiments, the nucleic acid fragment is a bait that includes an oligonucleotide between about 100 to 300 nucleotides, 130 and 230 nucleotides, or 150 and 200 nucleotides, in length.

In one embodiment, the nucleic acid fragment can be used to identify or capture, e.g., by hybridization, a LMNA-NTRK1 fusion. For example, the nucleic acid fragment can be a probe, a primer, or a bait, for use in identifying or capturing, e.g., by hybridization, a LMNA-NTRK1 fusion described herein. In one embodiment, the nucleic acid fragment can be useful for identifying or capturing a LMNA-NTRK1 breakpoint, e.g., the nucleotide sequence of: chromosome 1 at nucleotide plus or minus 10, 20, 30, 40, 50, 60, 80, 100, 150 nucleotides and chromosome 1 at nucleotide plus or minus 10, 20, 30, 40 50, 60, 80, 100, 150 nucleotides.

In one embodiment, the nucleic acid fragment hybridizes to a nucleotide sequence within a chromosomal rearrangement that creates an in-frame fusion of intron 2 or intron 9 or intron 11 or intron 12 of LMNA with intron 8 of NTRK1. In one embodiment, the nucleic acid fragment hybridizes to a nucleotide sequence in the region In other embodiments, the nucleic acid molecules includes a nucleotide sequence in the region of a nucleotide sequence of chromosome 1 coupled to (e.g., juxtaposed to) nucleotides in the region of a nucleotide sequence Y of chromosome 1. In one embodiment, the nucleic acid fragment hybridizes to a nucleotide sequence that includes a breakpoint. e.g., the nucleotide sequence of: chromosome 1 at nucleotide plus or minus 10, 20, 30, 40, 50, 60, 80, 100, 150 or more nucleotides and chromosome 1 at nucleotide plus or minus 10, 20, 30, 40 50, 60, 80, 100, 150 or more nucleotides. For example, the nucleic acid fragment can hybridize to a nucleotide sequence that includes the fusion junction between the LMNA gene and the NTRK1 gene, e.g., a nucleotide sequence that includes a portion of a nucleotide sequence within intron 2 or intron 9 or intron 11 or intron 12 of a LMNA gene and intron 2 of a NTRK1 gene.

In another embodiment, the nucleic acid fragment hybridizes to a nucleotide sequence that comprises at least 6, 12, 15, 20, 25, 50, 75, 100, 150 or more nucleotides from exon 2 or exon 9 or exon 11 or exon 12 of LMNA (e.g., from the nucleotide sequence of LMNA preceding the fusion junction with NTRK1, e.g., of the LMNA sequence shown in SEQ ID NO:70), and at least 6, 12, 15, 20, 25, 50, 75, 100, 150 or more nucleotides from exon 9 of NTRK1 (e.g., from the nucleotide sequence of NTRK1 following the fusion junction with LMNA, e.g., of the NTRK1 sequence shown in SEQ ID NO:3).

The probes or primers described herein can be used, for example, for FISH detection or PCR amplification. In one exemplary embodiment where detection is based on PCR, amplification of the LMNA-NTRK1 fusion junction fusion junction can be performed using a primer or a primer pair, e.g., for amplifying a sequence flanking the fusion junctions described herein, e.g., the mutations or the junction of a chromosomal rearrangement described herein, e.g., LMNA-NTRK1.

In one embodiment, a pair of isolated oligonucleotide primers can amplify a region containing or adjacent to a position in the LMNA-NTRK1 fusion. For example, forward primers can be designed to hybridize to a nucleotide sequence within LMNA genomic or mRNA sequence (e.g., a nucleotide sequence within exon 2 of LMNA of SEQ ID NO:70, and the reverse primers can be designed to hybridize to a nucleotide sequence of NTRK1 (e.g., a nucleotide sequence within exon 9 of NTRK1, of SEQ ID NO:3.

In another embodiment, the nucleic acid fragments can be used to identify, e.g., by hybridization, a LMNA-NTRK1 fusion. In one embodiment, the nucleic acid fragment hybridizes to a nucleotide sequence that includes a fusion junction between the LMNA transcript and the NTRK1 transcript.

In other embodiments, the nucleic acid fragment includes a bait that comprises a nucleotide sequence that hybridizes to a LMNA-NTRK1 fusion nucleic acid molecule described herein, and thereby allows the capture or isolation said nucleic acid molecule. In one embodiment, a bait is suitable for solution phase hybridization. In other embodiments, a bait includes a binding entity, e.g., an affinity tag, that allows capture and separation, e.g., by binding to a binding entity, of a hybrid formed by a bait and a nucleic acid hybridized to the bait.

In other embodiments, the nucleic acid fragment includes a library member comprising a LMNA-NTRK1 nucleic acid molecule described herein. In one embodiment, the library member includes a rearrangement that results in a LMNA-NTRK fusion described herein.

The nucleic acid fragment can be detectably labeled with, e.g., a radiolabel, a fluorescent label, a bioluminescent label, a chemiluminescent label, an enzyme label, a binding pair label, or can include an affinity tag; a tag, or identifier (e.g., an adaptor, barcode or other sequence identifier).

### LMNA-NTRK1 Fusion Polypeptides

In another embodiment, the LMNA-NTRK1 fusion comprises an amino acid sequence (e.g., a fragment of the amino acid sequence) shown in SEQ ID NO:71 and an amino acid sequence (e.g., a fragment of the amino acid sequence) shown in SEQ ID NO:4, or a fragment of the fusion. In one embodiment, the LMNA-NTRK1 fusion comprises an amino acid sequence substantially identical to the amino acid sequence (e.g., a fragment of the amino acid sequence) shown in SEQ ID NO:71 and the amino acid sequence (e.g., a fragment of the amino acid sequence) shown in SEQ ID NO:4, or a fragment thereof. In one embodiment, the LMNA-NTRK1 fusion comprises an amino acid sequence at least 70%, at least 75%, at least 80%, at least 85%, at least 90%, at least 95%, at least 99%, or at least 99.5, identical to the amino acid sequence (e.g., a fragment of the amino acid sequence) shown in SEQ ID NO:71 and the amino acid sequence (e.g., a fragment of the amino acid sequence) shown in SEQ ID NO:4. In one embodiment, the LMNA-NTRK1 fusion comprises a sequence containing at least 10, 20, 50, 100, 500, 600, 700, 800, 900, 1000, or more amino acids of the amino acid sequence shown in SEQ ID NO:71 and SEQ ID NO:4. In one embodiment, the LMNA-NTRK1 fusion comprises an amino acid sequence containing at least 5, 10, 20, 50, 100, 500, 600, 700, 800, 900, 1000, or more contiguous amino acids of the amino acid sequence shown in 156 SEQ ID NO:71 and at least 5, 10, 20, 50, 100, 500, 600, 700, 800, 900, 1000, or more contiguous amino acids of the amino acid sequence shown in SEQ ID NO:4. In one embodiment, the 5′ LMNA-3′ NTRK1 fusion polypeptide includes a receptor tyrosine kinase domain or a functional fragment thereof. In an embodiment, the 5′LMNA-3′NTRK1 fusion polypeptide comprises sufficient NTRK1 and sufficient LMNA sequence such that it has kinase activity, e.g., has elevated activity.

In one embodiment, the LMNA-NTRK1 fusion includes the full sequence or a fragment of the nucleotide sequence shown in SEQ ID NO:80, or a nucleotide sequence substantially identical thereto. In another embodiment, the LMNA-NTRK1 fusion polypeptide includes the full sequence or a fragment of the amino acid sequence shown in SEQ ID NO:81, or an amino acid sequence substantially identical thereto, for example at least 700% or at least 80% or at least 90% or more identical to this sequence or fragment.

In another aspect, the invention features a LMNA-NTRK1 fusion polypeptide (e.g., a purified LMNA-NTRK1 fusion polypeptide), a biologically active or antigenic fragment thereof, as well as reagents (e.g., antibody molecules that bind to a LMNA-NTRK1 fusion polypeptide), methods for modulating a LMNA-NTRK1 polypeptide activity and detection of a LMNA-NTRK1 polypeptide.

In one embodiment, the LMNA-NTRK1 fusion polypeptide has at least one biological activity. In one embodiment, at least one biological activity of the LMNA-NTRK1 fusion polypeptide is reduced or inhibited by an anti-cancer drug, e.g., a LMNA inhibitor, a NTRK1 inhibitor. In one embodiment, at least one biological activity of the LMNA-NTRK1 fusion polypeptide is reduced or inhibited by a NTRK1 inhibitor. In one embodiment, at least one biological activity of the LMNA-NTRK1 fusion polypeptide is reduced or inhibited by a LMNA inhibitor. In one embodiment, at least one biological activity of the LMNA-NTRK1 fusion polypeptide is reduced or inhibited by a NTRK1 inhibitor, e.g., lestaurtinib (CEP-701); AZ-23; indenopyrrolocarboazole 12a; oxindole 3; isothiazole 5n; thiazole 20h.

In yet other embodiments, the LMNA-NTRK1 fusion polypeptide is encoded by a nucleic acid molecule described herein. In one embodiment, the LMNA-NTRK1 fusion polypeptide is encoded by an in-frame fusion of intron 2 of LMNA with intron 8 of NTRK1 (e.g., a sequence on chromosome 1 and a sequence on chromosome 1). In another embodiment, the LMNA-NTRK1 fusion polypeptide includes an amino acid sequence encoded by a nucleotide sequence comprising a fusion junction between the LMNA transcript and the NTRK1 transcript.

In certain embodiments, the LMNA-NTRK1 fusion polypeptide comprises one or more of encoded exons 1-2 from LMNA and one or more of encoded exons 9-17 of NTRK1. In certain embodiments, the LMNA-NTRK1 fusion polypeptide comprises at least 1, 2 or more encoded exons of LMNA and at least at least 1, 2, 3, 4, 5, 6, 7, 8, 9 or more encoded exons of NTRK1. In certain embodiments, the LMNA-NTRK fusion polypeptide comprises a fusion of encoded exon 2 from LMNA and encoded exon 9 from NTRK1 (or a fragment thereof). In other embodiments, the fusion comprises at least 1, 2 or more encoded exons of LMNA; and at least 1, 2, 3, 4, 5, 6, 7, 8, 9 or more encoded exons of NTRK1. In certain embodiments, the LMNA-NTRK1 fusion polypeptide comprises encoded exons 1-2 from LMNA and exons 9-17 of NTRK1. In certain embodiments, the 5′ LMNA-3′ NTRK1 fusion polypeptide comprises a fusion junction of the sequence of exon 2 from LMNA and the sequence of exon 9 from NTRK1.

In certain embodiments, the LMNA-NTRK11 fusion comprises the amino acid sequence corresponding to exon 2 or a fragment thereof from LMNA, and the amino acid sequence corresponding to exon 9 or a fragment thereof from NTRK1 (e.g., as shown in SEQ ID NO:71 and SEQ ID NO:4). In one embodiment, the LMNA-NTRK1 fusion comprises at least 5, 10, 15, 20 or more amino acids from exon 2 of LMNA (e.g., from the amino acid sequence of LMNA preceding the fusion junction with NTRK1, e.g., of the LMNA sequence shown in SEQ ID NO:71), and at least 5, 10, 15, 20 or more amino acids from exon 9 of NTRK1 (e.g., from the amino acid sequence of NTRK1 following the fusion junction with LMNA, e.g., of the NTRK1 sequence shown in SEQ ID NO:4).

In one embodiment, the LMNA-NTRK1 fusion polypeptide includes a tyrosine kinase domain or a functional fragment thereof. In a related aspect, the invention features LMNA-NTRK1 fusion polypeptide or fragments operatively linked to heterologous polypeptides to form fusion proteins.

In another embodiment, the LMNA-NTRK1 fusion polypeptide or fragment is a peptide, e.g., an immunogenic peptide or protein containing a fusion junction described herein. Such immunogenic peptides or proteins can be used to raise antibodies specific to the fusion protein. In other embodiments, such immunogenic peptides or proteins can be used for vaccine preparation. The vaccine preparation can include other components, e.g., an adjuvant.

In another aspect, the invention features antibody molecules that bind to a LMNA-NTRK1 fusion polypeptide or fragment described herein. In embodiments, the antibody can distinguish wild type NTRK1 (or LMNA) from LMNA-NTRK1.

### Detection Reagents and Detection of Mutations

In another aspect, the invention features a detection reagent, e.g., a purified or an isolated preparation thereof. Detection reagents can distinguish a nucleic acid, or protein sequence, having a breakpoint, e.g., a LMNA-NTRK1 breakpoint; from a reference sequence. In one embodiment, the detection reagent detects (e.g., specifically detects) a LMNA-NTRK1 fusion nucleic acid or a polypeptide, e.g., distinguishes a wild type NTRK1 or another NTRK1 fusion (or LMNA) from a LMNA-NTRK1 nucleic acid (e.g., as described herein in SEQ ID NO:70 and SEQ ID NO:3); or a LMNA-NTRK1 polypeptide (e.g., as described herein in SEQ ID NO:71 and SEQ ID NO:4).

Detection reagents, e.g., nucleic acid-based detection reagents, can be used to identify mutations in a target nucleic acid, e.g., DNA, e.g., genomic DNA or cDNA, or RNA, e.g., in a sample, e.g., a sample of nucleic acid derived from a neoplastic or tumor cell, e.g., a melanocytic neoplasm, melanoma or metastatic cell. Detection reagents, e.g., antibody-based detection reagents, can be used to identify mutations in a target protein, e.g., in a sample, e.g., a sample of protein derived from, or produced by, a neoplastic or tumor cell, e.g., a melanocytic neoplasm, melanoma or metastatic cell.

### Method of Treatment

In a related aspect, a method of inhibiting, reducing, or treating a hyperproliferative disorder, e.g., a neoplasm (including benign, pre-malignant or malignant (e.g., a cancer), in a subject is provided. The method includes administering to the subject a preselected therapeutic agent, e.g., an anti-cancer agent (e.g., a kinase inhibitor), as a single agent, or in combination, in an amount sufficient to reduce, inhibit or treat the activity or expression of LMNA-NTRK1 (e.g., a LMNA-NTRK1 fusion described herein), thereby inhibiting, reducing, or treating the hyperproliferative disorder in the subject.

In one embodiment, the subject treated has a LMNA-NTRK1 fusion; e.g., the subject has a tumor or cancer harboring a LMNA-NTRK1 fusion. In other embodiments, the subject has been previously identified as having a LMNA-NTRK1 fusion. In yet other embodiments, the subject has been previously identified as being likely or unlikely to respond to treatment with a protein kinase inhibitor, e.g., a subject that has previously participated in a clinical trial. In other embodiments, the subject has been previously identified as being likely or unlikely to respond to treatment with a protein kinase inhibitor, based on the presence of the LMNA-NTRK1 fusion. In one embodiment, the subject is a mammal, e.g., a human. In one embodiment, the subject has, or at risk of having a cancer at any stage of disease. In other embodiments, the subject is a patient, e.g., a cancer patient.

In certain embodiments, the neoplasm or neoplastic cell is a benign, pre-malignant, malignant (cancer) or metastasis. In certain embodiments, the cancer is a solid tumor, a soft tissue tumor, or a metastatic lesion.

In one embodiment, the neoplasm or neoplastic cell is a benign, pre-malignant, malignant (cancer) or metastasis. In certain embodiments, the cancer is a solid tumor, a soft tissue tumor, or a metastatic lesion. In one embodiment, the cancer is a carcinoma. In one embodiment, the cancer is an adenocarcinoma. In an embodiment, the cancer is a lung adenocarcinoma. In one embodiment, the cancer is chosen from a lung cancer, a pancreatic cancer, melanoma, a colorectal cancer, an esophageal-gastric cancer, a thyroid cancer, or an adenocarcinoma. In one embodiment, the cancer is a thyroid cancer. In one embodiment, the cancer is a papillary thyroid carcinoma. In one embodiment, the cancer is a lung adenocarcinoma. In other embodiment, the lung cancer is chosen from one or more of the following: non-small cell lung cancer (NSCLC), small cell lung cancer (SCLC), squamous cell carcinoma (SCC), adenocarcinoma of the lung, bronchogenic carcinoma, or a combination thereof. In one embodiment, the lung cancer is NSCLC or SCC.

In one embodiment, the anti-cancer agent is a NTRK1 inhibitor. In one embodiment, the anti-cancer agent is a LMNA inhibitor. In one embodiment, the anti-cancer agent is a kinase inhibitor. In one embodiment, the anti-cancer agent is a NTRK1 inhibitor, e.g., lestaurtinib (CEP-701); AZ-23; indenopyrrolocarboazole 12a; oxindole 3; isothiazole 5n; thiazole 20h.

### LMNA-RET Fusions

In one embodiment, a fusion includes an in-frame fusion of an exon of Lamin A/C (LMNA), e.g., one more exons of LMNA (e.g., one or more of exons 1-2 LMNA) or a fragment thereof, and an exon of ret proto-oncogene (RET). e.g., one or more exons of a RET (e.g., one or more of exons 12-19 of RET) or a fragment thereof. For example, the LMNA-RET fusion can include an in-frame fusion within an intron of LMNA (e.g., intron 2) or a fragment thereof, with an intron of RET (e.g., intron 8) or a fragment thereof. In one embodiment, the fusion of the LMNA-RET fusion comprises the nucleotide sequence of: chromosome 1 at one or more of a nucleotide (plus or minus 10, 20, 30, 50, 60, 70, 80, 100 or more nucleotides) and chromosome 1 at one or more of a nucleotide (plus or minus 10, 20, 30, 50, 60, 70, 80, 100 or more nucleotides). In one embodiment, the LMNA-RET fusion is a translocation, e.g., a translocation of a portion of chromosome 1 and a portion of chromosome 10.

In certain embodiments, the LMNA-RET fusion is in a 5′-LMNA to 3′-RET configuration (also referred to herein as “5′-LMNA-RET-3′).” The term “fusion” or “fusion molecule” can refer to a polypeptide or a nucleic acid fusion, depending on the context. It may include a full-length sequence of a fusion or a fragment thereof, e.g., a fusion junction (e.g., a fragment including a portion of LMNA and a portion of RET, e.g., a portion of the LMNA-RET fusion described herein). In one embodiment, the LMNA-RET fusion polypeptide includes a fragment of the amino acid sequence shown in SEQ ID NO:71 and a fragment of the amino acid sequence shown in SEQ ID NO:6, or an amino acid sequence substantially identical thereto. In another embodiment, the LMNA-RET fusion nucleic acid includes a fragment of the nucleotide sequence shown in SEQ ID NO:70 and a fragment of the nucleotide sequence shown in SEQ ID NO:5, or a nucleotide sequence substantially identical thereto. In one embodiment, the LMNA-RET fusion polypeptide comprises sufficient LMNA and sufficient RET sequence such that the 5′ LMNA-3′ RET fusion has kinase activity, e.g., has elevated activity, e.g., tyrosine kinase activity. In any event, the fusion causes activation in the cells harboring it of oncogenic signaling pathways.

In certain embodiments, the LMNA-RET fusion comprises one or more (or all of) exons 1-2 from LMNA and one or more (or all of) exons 12-19 of RET (e.g., one or more of the exons shown in SEQ ID NO:70 and SEQ ID NO:5. In another embodiment, the LMNA-RET fusion comprises one or more (or all of) exons 1-2 of LMNA and one or more (or all of) exons 12-19 of RET. In certain embodiments, the LMNA-RET fusion comprises at least 1, 2 or more exons (or encoded exons) from LMNA and at least 1, 2, 3, 4, 5, 6, 7, 8, 9 or more exons (or encoded exons) from RET (e.g., from the LMNA and RET sequences shown in SEQ ID NO:70 and SEQ ID NO:71 and SEQ ID NO:5 and SEQ ID NO:6.

In certain embodiments, the LMNA-RET fusion comprises exons 1-2 or a fragment thereof from LMNA, and exons 12-19 or a fragment thereof from RET (e.g., as shown in SEQ ID NO:70 and SEQ ID NO:5). In one embodiment, the LMNA-RET fusion comprises at least 5, 10, 15, 20, 30, 40, 50 or more amino acids from exons 1-2 of LMNA (e.g., from the amino acid sequence of LMNA as shown in SEQ ID NO:71 (e.g., from the amino acid sequence of LMNA preceding the fusion junction with RET, and at least 5, 10, 15, 20, 30, 40, 50 or more amino acids from exons 12-19 of RET (e.g., from the amino acid sequence of RET as shown SEQ ID NO:6). In another embodiment, the LMNA-RET fusion comprises at least 6, 12, 15, 20, 25, 50, 75, 100 or more nucleotides from exons 1-2 of LMNA (e.g., from the nucleotide sequence of LMNA as shown in SEQ ID NO:70 (e.g., from the nucleotide sequence of LMNA preceding the fusion junction with RET); and at least 6, 12, 15, 20, 25, 50, 75, 100 or more nucleotides from exons 12-19 of RET (e.g., from the nucleotide sequence of RET as shown in SEQ ID NO:5).

In one embodiment, the LMNA-RET fusion includes the full sequence or a fragment of the nucleotide sequence shown in SEQ ID NO:110, or a nucleotide sequence substantially identical thereto. In another embodiment, the LMNA-RET fusion polypeptide includes the full sequence or a fragment of the amino acid sequence shown in SEQ ID NO: 111, or an amino acid sequence substantially identical thereto. The sequences SEQ ID NO:110 and SEQ ID NO: 111 have been predicted from sequencing information by reference to NM_{—}005572 for protein LMNA and NM_{—}020975 for kinase RET.

### LMNA-RET Nucleic Acid Molecules

In one aspect, the invention features a nucleic acid molecule (e.g., an isolated or purified) nucleic acid molecule that includes a fragment of a LMNA gene and a fragment of a RET gene. In one embodiment, the nucleotide sequence encodes a LMNA-RET fusion polypeptide that includes a tyrosine kinase domain or a functional fragment thereof. In another embodiment, the nucleotide sequence encodes a fragment of the RET polypeptide including the amino acid sequence of SEQ ID NO:6 or a fragment thereof, or a sequence substantially identical thereto.

In one embodiment, the nucleic acid molecule includes a fusion. e.g., an in-frame fusion, between an intron of LMNA (e.g., intron 2 or a fragment thereof), and an intron of RET (e.g., intron 8, or a fragment thereof). The LMNA-RET fusion can comprise a fusion of the nucleotide sequence of: chromosome 1 at one or more of a nucleotide (plus or minus 10, 20, 30, 50, 60, 70, 80, 100 nucleotides) and chromosome 1 at one or more of a nucleotide (plus or minus 10, 20, 30, 50, 60, 70, 80, 100 nucleotides), or a fragment thereof. In one embodiment, the LMNA-RET fusion comprises a fusion of the nucleotide sequence of: chromosome 1 at one or more of a nucleotide (plus or minus 10, 20, 30, 50, 60, 70, 80, 100 nucleotides) and chromosome 10 at one or more of a nucleotide (plus or minus 10, 20, 30, 50, 60, 70, 80, 100 nucleotides), or a fragment thereof.

In another embodiment, the LMNA-RET fusion comprises a nucleotide sequence (e.g., a fragment of a nucleotide sequence) shown in SEQ ID NO:70 and a nucleotide sequence (e.g., a fragment of a nucleotide sequence) shown in SEQ ID NO:5, or a fragment of the fusion. In one embodiment, the LMNA-RET fusion comprises a nucleotide sequence substantially identical to the nucleotide sequence (e.g., a fragment of a nucleotide sequence) shown in SEQ ID NO:70 and the nucleotide sequence (e.g., a fragment of a nucleotide sequence) shown SEQ ID NO:5, or a fragment of the fusion. In one embodiment, the LMNA-RET fusion comprises a nucleotide sequence at least 70%, at least 75%, at least 80%, at least 85%, at least 90%, at least 95%, at least 99%, at least 99.5 or greater, identical to the nucleotide sequence (e.g., a fragment of a nucleotide sequence) shown in SEQ ID NO:70 and to the nucleotide sequence (e.g., a fragment of a nucleotide sequence) shown in SEQ ID NO:5. In one embodiment, the LMNA-RET fusion comprises a nucleotide sequence containing at least 25, 50, 100, 150, 200, 500, 1000, 1500, 2000, 2500, 3000, or more nucleotides of the nucleotide sequence shown in SEQ ID NO:70 and a nucleotide sequence containing at least 25, 50, 100, 150, 200, 500, 1000, 1500, 2000, 2500, 3000, or more nucleotides of the nucleotide sequence shown in SEQ ID NO:5. In one embodiment, the LMNA-RET fusion comprises a nucleotide sequence containing at least 25, 50, 100, 150, 200, 500, 1000, 1500, 2000, 2500, 3000, or more contiguous nucleotides of the nucleotide sequence shown in 155 SEQ ID NO:70 and a nucleotide sequence containing at least 25, 50, 100, 150, 200, 500, 1000, 1500, 2000, 2500, 3000, or more nucleotides of the nucleotide sequence shown in SEQ ID NO:5.

In another embodiment, the nucleic acid molecule includes a fusion, e.g., an in-frame fusion, of at least 1, 2 or more exons of LMNA or a fragment thereof (e.g., one or more of exons 1-2 of LMNA or a fragment thereof), and at least 1, 2, 3, 4, 5, 6, 7, 8, 9 or more exons of RET or a fragment thereof (e.g., one or more of exons 12-19 of RET or a fragment thereof). In yet other embodiments, the nucleic acid molecule includes a fragment the nucleotide sequence shown in SEQ ID NO:70 and a fragment of the nucleotide sequence shown in SEQ ID NO:5 or a fragment of the fusion, or a sequence substantially identical thereto.

In one embodiment, the nucleic acid molecule is complementary to at least a portion of a nucleotide sequence disclosed herein, e.g., is capable of hybridizing under a stringency condition described herein to SEQ ID NO:70 and/or SEQ ID NO:5, or a fragment thereof. In yet another embodiment, the nucleic acid molecule hybridizes to a nucleotide sequence that is complementary to at least a portion of a nucleotide sequence disclosed herein, e.g., is capable of hybridizing under a stringency condition to a nucleotide sequence complementary to SEQ ID NO:70 and/or SEQ ID NO:5, or a fragment thereof. The nucleotide sequence of a cDNA encoding an exemplary 5′ LMNA-3′ RET fusion is shown in at least exon 2 (e.g., exons 1-2) of SEQ ID NO:70 and at least exon 9 (e.g., exons 12-19) of SEQ ID NO:5, and the predicted amino acid sequence is shown in the corresponding encoded exons of SEQ ID NO:71 and the corresponding encoded exons of SEQ ID NO:6, respectively.

In an embodiment the LMNA-RET nucleic acid molecule comprises sufficient LMNA and sufficient RET sequence such that the encoded 5′ LMNA-3′ RET fusion has kinase activity, e.g., has elevated activity. In certain embodiments, the 5′ LMNA-3′ RET fusion comprises exons 1-2 from LMNA and exons 12-19 from RET. In certain embodiments, the LMNA-RET fusion comprises at least 1, 2 or more exons from LMNA and at least at least 1, 2, 3, 4, 5, 6, 7, 8, 9 or more exons of RET. In certain embodiments, the LMNA-RET fusion comprises a fusion of exon 2 from LMNA and exon 9 from RET. In another embodiment, the LMNA-RET fusion comprises 1, 2 or more exons of LMNA; and at least 1, 2, 3, 4, 5, 6, 7, 8, 9 or more exons of RET.

In one embodiment, the nucleic acid molecule includes a nucleotide sequence that has an in-frame fusion of intron 2 of LMNA (e.g., NM_{—}001126113) with intron 8 of RET (e.g., NM_{—}020630). In another embodiment, the nucleic acid molecule includes a nucleotide sequence that includes a breakpoint. For example, the nucleic acid molecule includes a nucleotide sequence that includes the fusion junction between the LMNA gene and the RET gene, e.g., the breakpoint between intron 2 or intron 9 or intron 11 or intron 12 of LMNA and intron 2 of RET. In other embodiments, the nucleic acid molecules includes a nucleotide sequence of one or more of nucleotide of chromosome 1 coupled to (e.g., directly or indirectly juxtaposed to) one or more of nucleotide of chromosome 1. In one embodiment, the nucleic acid molecule includes the nucleotide sequence of: chromosome 1 at one or more of a nucleotide plus or minus 10, 20, 30, 40 50, 60, 80, 100, 150 nucleotides and chromosome 1 at one or more of a nucleotide plus or minus 10, 20, 30, 40 50, 60, 80, 100, 150 nucleotides (corresponding to the breakpoint of a LMNA-RET fusion), or a fragment thereof, or a sequence substantially identical thereto. In one embodiment, the nucleic acid molecule is complementary to at least a portion of a nucleotide sequence disclosed herein, e.g., is capable of hybridizing under a stringency condition described herein to SEQ ID NO:70 and/or SEQ ID NO:5 or a fragment thereof. In yet other embodiment, the nucleic acid molecule hybridizes to a nucleotide sequence that is complementary to at least a portion of a nucleotide sequence disclosed herein, e.g., is capable of hybridizing under a stringency condition described herein to a nucleotide sequence complementary to SEQ ID NO:70 or SEQ ID NO:5 or a fragment thereof.

In another embodiment, the LMNA-RET fusion nucleic acid comprises at least 6, 12, 15, 20, 25, 50, 75, 100 or more nucleotides from exon 2 or exon 9 or exon 11 or exon 12 of LMNA (e.g., from the nucleotide sequence of LMNA preceding the fusion junction with RET, e.g., of the LMNA sequence shown in SEQ ID NO:70), and at least 6, 12, 15, 20, 25, 50, 75, 100 or more nucleotides from exon 9 of RET (e.g., from the nucleotide sequence of RET following the fusion junction with LMNA. e.g., of the RET sequence shown in SEQ ID NO:5).

In other embodiments, the nucleic acid molecule includes a nucleotide sequence encoding a LMNA-RET fusion polypeptide that includes a fragment of a LMNA gene and a fragment of a RET gene. In one embodiment, the nucleotide sequence encodes a LMNA-RET fusion polypeptide that includes e.g., a tyrosine kinase domain or a functional fragment thereof. In yet other embodiments, the nucleic acid molecule includes a nucleotide sequence encoding the amino acid sequence (e.g., a fragment of the amino acid sequence) shown in SEQ ID NO:71 and a nucleotide sequence encoding the amino acid sequence (e.g., a fragment of the amino acid sequence) shown in SEQ ID NO:6, or a fragment of the fusion, or a sequence substantially identical thereto. In one embodiment, the encoded LMNA-RET fusion polypeptide includes a tyrosine kinase domain or a functional fragment thereof.

In a related aspect, the invention features nucleic acid constructs that include the LMNA-RET nucleic acid molecules described herein. In certain embodiments, the nucleic acid molecules are operatively linked to a native or a heterologous regulatory sequence. Also included are vectors and host cells that include the LMNA-RET nucleic acid molecules described herein, e.g., vectors and host cells suitable for producing the nucleic acid molecules and polypeptides described herein.

In a related aspect, methods of producing the nucleic acid molecules and polypeptides described herein are also described.

In another aspect, the invention features nucleic acid molecules that reduce or inhibit the expression of a nucleic acid molecule that encodes a LMNA-RET fusion described herein. Examples of such nucleic acid molecules include, for example, antisense molecules, ribozymes, RNAi, triple helix molecules that hybridize to a nucleic acid encoding LMNA-RET, or a transcription regulatory region of LMNA-RET, and blocks or reduces mRNA expression of LMNA-RET.

### Nucleic Acid Detection and Capturing Reagents

The invention also features a nucleic acid molecule, e.g., nucleic acid fragment, suitable as probe, primer, bait or library member that includes, flanks, or hybridizes to the LMNA-RET fusions described herein. Such nucleic acid molecules are useful for identifying, or are otherwise based on, the LMNA-RET fusions described herein. In certain embodiments, the probe, primer or bait molecule is an oligonucleotide that allows capture, detection or isolation of a LMNA-RET fusion nucleic acid molecule described herein. The oligonucleotide can comprise a nucleotide sequence substantially complementary to a fragment of the LMNA-RET fusion nucleic acid molecules described herein. The sequence identity between the nucleic acid fragment, e.g., the oligonucleotide, and the target LMNA-RET sequence need not be exact, so long as the sequences are sufficiently complementary to allow the capture, detection or isolation of the target sequence. In one embodiment, the nucleic acid fragment is a probe or primer that includes an oligonucleotide between about 5 and 25, e.g., between 10 and 20, or 10 and 15 nucleotides in length. In other embodiments, the nucleic acid fragment is a bait that includes an oligonucleotide between about 100 to 300 nucleotides, 130 and 230 nucleotides, or 150 and 200 nucleotides, in length.

In one embodiment, the nucleic acid fragment can be used to identify or capture, e.g., by hybridization, a LMNA-RET fusion. For example, the nucleic acid fragment can be a probe, a primer, or a bait, for use in identifying or capturing, e.g., by hybridization, a LMNA-RET fusion described herein. In one embodiment, the nucleic acid fragment can be useful for identifying or capturing a LMNA-RET breakpoint, e.g., the nucleotide sequence of: chromosome 1 at nucleotide plus or minus 10, 20, 30, 40, 50, 60, 80, 100, 150 nucleotides and chromosome 1 at nucleotide plus or minus 10, 20, 30, 40 50, 60, 80, 100, 150 nucleotides.

In one embodiment, the nucleic acid fragment hybridizes to a nucleotide sequence within a chromosomal rearrangement that creates an in-frame fusion of intron 2 or intron 9 or intron 1 or intron 12 of LMNA with intron 8 of RET. In one embodiment, the nucleic acid fragment hybridizes to a nucleotide sequence in the region In other embodiments, the nucleic acid molecules includes a nucleotide sequence in the region of a nucleotide sequence of chromosome 1 coupled to (e.g., juxtaposed to) nucleotides in the region of a nucleotide sequence of chromosome 10. In one embodiment, the nucleic acid fragment hybridizes to a nucleotide sequence that includes a breakpoint, e.g., the nucleotide sequence of: chromosome 1 at nucleotide plus or minus 10, 20, 30, 40, 50, 60, 80, 100, 150 or more nucleotides and chromosome 1 at nucleotide plus or minus 10, 20, 30, 40 50, 60, 80, 100, 150 or more nucleotides. For example, the nucleic acid fragment can hybridize to a nucleotide sequence that includes the fusion junction between the LMNA gene and the RET gene, e.g., a nucleotide sequence that includes a portion of a nucleotide sequence within intron 2 or intron 9 or intron 11 or intron 12 of a LMNA gene and intron 2 of a RET gene.

In another embodiment, the nucleic acid fragment hybridizes to a nucleotide sequence that comprises at least 6, 12, 15, 20, 25, 50, 75, 100, 150 or more nucleotides from exon 2 or exon 9 or exon 11 or exon 12 of LMNA (e.g., from the nucleotide sequence of LMNA preceding the fusion junction with RET, e.g., of the LMNA sequence shown in SEQ ID NO:70), and at least 6, 12, 15, 20, 25, 50, 75, 100, 150 or more nucleotides from exon 9 of RET (e.g., from the nucleotide sequence of RET following the fusion junction with LMNA, e.g., of the RET sequence shown in SEQ ID NO:5).

The probes or primers described herein can be used, for example, for FISH detection or PCR amplification. In one exemplary embodiment where detection is based on PCR, amplification of the LMNA-RET fusion junction fusion junction can be performed using a primer or a primer pair, e.g., for amplifying a sequence flanking the fusion junctions described herein, e.g., the mutations or the junction of a chromosomal rearrangement described herein, e.g., LMNA-RET.

In one embodiment, a pair of isolated oligonucleotide primers can amplify a region containing or adjacent to a position in the LMNA-RET fusion. For example, forward primers can be designed to hybridize to a nucleotide sequence within LMNA genomic or mRNA sequence (e.g., a nucleotide sequence within exon 2 of LMNA of SEQ ID NO:70, and the reverse primers can be designed to hybridize to a nucleotide sequence of RET (e.g., a nucleotide sequence within exon 9 of RET, of SEQ ID NO:5.

In another embodiment, the nucleic acid fragments can be used to identify. e.g., by hybridization, a LMNA-RET fusion. In one embodiment, the nucleic acid fragment hybridizes to a nucleotide sequence that includes a fusion junction between the LMNA transcript and the RET transcript.

In other embodiments, the nucleic acid fragment includes a bait that comprises a nucleotide sequence that hybridizes to a LMNA-RET fusion nucleic acid molecule under stringent conditions as described herein, and thereby allows the capture or isolation said nucleic acid molecule. In one embodiment, a bait is suitable for solution phase hybridization. In other embodiments, a bait includes a binding entity, e.g., an affinity tag, that allows capture and separation, e.g., by binding to a binding entity, of a hybrid formed by a bait and a nucleic acid hybridized to the bait.

In other embodiments, the nucleic acid fragment includes a library member comprising a LMNA-RET nucleic acid molecule described herein. In one embodiment, the library member includes a rearrangement that results in a LMNA-RET fusion described herein.

The nucleic acid fragment can be detectably labeled with, e.g., a radiolabel, a fluorescent label, a bioluminescent label, a chemiluminescent label, an enzyme label, a binding pair label, or can include an affinity tag; a tag, or identifier (e.g., an adaptor, barcode or other sequence identifier).

### LMNA-RET Fusion Polypeptides

In another embodiment, the LMNA-RET fusion comprises an amino acid sequence (e.g., a fragment of the amino acid sequence) shown in SEQ ID NO:71 and an amino acid sequence (e.g., a fragment of the amino acid sequence) shown in SEQ ID NO:6, or a fragment of the fusion. In one embodiment, the LMNA-RET fusion comprises an amino acid sequence substantially identical to the amino acid sequence (e.g., a fragment of the amino acid sequence) shown in SEQ ID NO:71290 and the amino acid sequence (e.g., a fragment of the amino acid sequence) shown in SEQ ID NO:6, or a fragment thereof. In one embodiment, the LMNA-RET fusion comprises an amino acid sequence at least 70%, at least 75%, at least 80%, at least 85%, at least 90%, at least 95%, at least 99%, at least 99.5 or greater, identical to the amino acid sequence (e.g., a fragment of the amino acid sequence) shown in SEQ ID NO:71 and the amino acid sequence (e.g., a fragment of the amino acid sequence) shown in SEQ ID NO:6. In one embodiment, the LMNA-RET fusion comprises a sequence containing at least 10, 20, 50, 100, 500, 600, 700, 800, 900, 1000, or more amino acids of the amino acid sequence shown in SEQ ID NO:71 and SEQ ID NO:6. In one embodiment, the LMNA-RET fusion comprises an amino acid sequence containing at least 5, 10, 20, 50, 100, 500, 600, 700, 800, 900, 1000, or more contiguous amino acids of the amino acid sequence shown in 156 SEQ ID NO:71 and at least 5, 10, 20, 50, 100, 500, 600, 700, 800, 900, 1000, or more contiguous amino acids of the amino acid sequence shown in SEQ ID NO:6. In one embodiment, the 5′ LMNA-3′ RET fusion polypeptide includes a receptor tyrosine kinase domain or a functional fragment thereof. In an embodiment, the 5′LMNA-3′RET fusion polypeptide comprises sufficient RET and sufficient LMNA sequence such that it has kinase activity, e.g., has elevated activity.

In one embodiment, the LMNA-RET fusion includes the full sequence or a fragment of the nucleotide sequence shown in SEQ ID NO:110 or a nucleotide sequence substantially identical thereto. In another embodiment, the LMNA-RET fusion polypeptide includes the full sequence or a fragment of the amino acid sequence shown in SEQ ID NO: 111, or an amino acid sequence substantially identical thereto.

In another aspect, the invention features a LMNA-RET fusion polypeptide (e.g., a purified LMNA-RET fusion polypeptide), a biologically active or antigenic fragment thereof, as well as reagents (e.g., antibody molecules that bind to a LMNA-RET fusion polypeptide), methods for modulating a LMNA-RET polypeptide activity and detection of a LMNA-RET polypeptide.

In one embodiment, the LMNA-RET fusion polypeptide has at least one biological activity. In one embodiment, at least one biological activity of the LMNA-RET fusion polypeptide is reduced or inhibited by an anti-cancer drug, e.g., a LMNA inhibitor, a RET inhibitor. In one embodiment, at least one biological activity of the LMNA-RET fusion polypeptide is reduced or inhibited by a RET inhibitor. In one embodiment, at least one biological activity of the LMNA-RET fusion polypeptide is reduced or inhibited by a LMNA inhibitor. In one embodiment, at least one biological activity of the LMNA-RET fusion polypeptide is reduced or inhibited by a RET inhibitor. e.g., lestaurtinib (CEP-701); AZ-23; indenopyrrolocarboazole 12a; oxindole 3; isothiazole 5n; thiazole 20h.

In yet other embodiments, the LMNA-RET fusion polypeptide is encoded by a nucleic acid molecule described herein. In one embodiment, the LMNA-RET fusion polypeptide is encoded by an in-frame fusion of intron 2 of LMNA with intron 8 of RET (e.g., a sequence on chromosome 1 and a sequence on chromosome 1). In another embodiment, the LMNA-RET fusion polypeptide includes an amino acid sequence encoded by a nucleotide sequence comprising a fusion junction between the LMNA transcript and the RET transcript.

In certain embodiments, the LMNA-RET fusion polypeptide comprises one or more of encoded exons 1-2 from LMNA and one or more of encoded exons 12-19 of RET. In certain embodiments, the LMNA-RET fusion polypeptide comprises at least 1, 2 or more encoded exons of LMNA and at least at least 1, 2, 3, 4, 5, 6, 7, 8, 9 or more encoded exons of RET. In certain embodiments, the LMNA-RET fusion polypeptide comprises a fusion of encoded exon 2 from LMNA and encoded exon 9 from RET (or a fragment thereof). In other embodiments, the fusion comprises at least 1, 2 or more encoded exons of LMNA; and at least 1, 2, 3, 4, 5, 6, 7, 8, 9 or more encoded exons of RET. In certain embodiments, the LMNA-RET fusion polypeptide comprises encoded exons 1-2 from LMNA and exons 12-19 of RET. In certain embodiments, the 5′ LMNA-3′ RET fusion polypeptide comprises a fusion junction of the sequence of exon 2 from LMNA and the sequence of exon 9 from RET.

In certain embodiments, the LMNA-RET fusion comprises the amino acid sequence corresponding to exon 2 or a fragment thereof from LMNA, and the amino acid sequence corresponding to exon 9 or a fragment thereof from RET (e.g., as shown in SEQ ID NO:71 and SEQ ID NO:6). In one embodiment, the LMNA-RET fusion comprises at least 5, 10, 15, 20 or more amino acids from exon 2 of LMNA (e.g., from the amino acid sequence of LMNA preceding the fusion junction with RET, e.g., of the LMNA sequence shown in SEQ ID NO:71), and at least 5, 10, 15, 20 or more amino acids from exon 9 of RET (e.g., from the amino acid sequence of RET following the fusion junction with LMNA, e.g., of the RET sequence shown in SEQ ID NO:6).

In one embodiment, the LMNA-RET fusion polypeptide includes a tyrosine kinase domain or a functional fragment thereof. In a related aspect, the invention features LMNA-RET fusion polypeptides or fragments operatively linked to heterologous polypeptides to form fusion proteins.

In another embodiment, the LMNA-RET fusion polypeptide or fragment is a peptide, e.g., an immunogenic peptide or protein. The peptide contains a fusion junction described herein. Such immunogenic peptides or proteins can be used to raise antibodies specific to the fusion protein. Such antibodies can serve to identify the fusion. In other embodiments, such immunogenic peptides or proteins can be used for vaccine preparation. The vaccine preparation can include other components, e.g., an adjuvant.

In another aspect, the invention features antibody molecules that bind to a LMNA-RET fusion polypeptide or fragment described herein. In some embodiments, the antibody can distinguish wild type RET (or LMNA) from LMNA-RET.

### Detection Reagents and Detection of Mutations

In another aspect, the invention features a detection reagent, e.g., a purified or an isolated preparation thereof. Detection reagents can distinguish a nucleic acid, or protein sequence, having a breakpoint, e.g., a LMNA-RET breakpoint; from a reference sequence. In one embodiment, the detection reagent detects (e.g., specifically detects) a LMNA-RET fusion nucleic acid or a polypeptide, e.g., distinguishes a wild type RET or another RET fusion (or LMNA) from a LMNA-RET nucleic acid (e.g., as described herein in SEQ ID NO:70 and SEQ ID NO:5); or a LMNA-RET polypeptide (e.g., as described herein in SEQ ID NO:71 and SEQ ID NO:6).

Detection reagents, e.g., nucleic acid-based detection reagents, can be used to identify mutations in a target nucleic acid, e.g., DNA, e.g., genomic DNA or cDNA, or RNA. e.g., in a sample, e.g., a sample of nucleic acid derived from a neoplastic or tumor cell, e.g., a melanocytic neoplasm, melanoma or metastatic cell. Detection reagents, e.g., antibody-based detection reagents, can be used to identify mutations in a target protein, e.g., in a sample, e.g., a sample of protein derived from, or produced by, a neoplastic or tumor cell, e.g., a melanocytic neoplasm, melanoma or metastatic cell.

### Method of Treatment

In a related aspect, a method of inhibiting, reducing, or treating a hyperproliferative disorder, e.g., a neoplasm (including benign, pre-malignant or malignant (e.g., a cancer), in a subject is provided. The method includes administering to the subject a preselected therapeutic agent, e.g., an anti-cancer agent (e.g., a kinase inhibitor), as a single agent, or in combination, in an amount sufficient to reduce, inhibit or treat the activity or expression of LMNA-RET (e.g., a LMNA-RET fusion described herein), thereby inhibiting, reducing, or treating the hyperproliferative disorder in the subject.

In one embodiment, the subject treated has a LMNA-RET fusion; e.g., the subject has a tumor or cancer harboring a LMNA-RET fusion. In other embodiments, the subject has been previously identified as having a LMNA-RET fusion. In yet other embodiments, the subject is or has been previously identified as being likely or unlikely to respond to treatment with a protein kinase inhibitor, e.g., a subject that has previously participated in a clinical trial or a subject that is being tested for the presence of a fusion as described herein. In other embodiments, the subject is or has been previously identified as being likely or unlikely to respond to treatment with a protein kinase inhibitor, based on the presence of the LMNA-RET fusion. In one embodiment, the subject is a mammal, e.g., a human. In one embodiment, the subject has, or is at risk of having a cancer at any stage of disease. In other embodiments, the subject is a patient, e.g., a cancer patient.

In certain embodiments, the neoplasm or neoplastic cell is a benign, pre-malignant, malignant (cancer) or metastasis. In certain embodiments, the cancer is a solid tumor, a soft tissue tumor, or a metastatic lesion.

In one embodiment, the neoplasm or neoplastic cell is a benign, pre-malignant, malignant (cancer) or metastasis. In certain embodiments, the cancer is a solid tumor, a soft tissue tumor, or a metastatic lesion. In one embodiment, the cancer is a carcinoma. In one embodiment, the cancer is an adenocarcinoma. In an embodiment, the cancer is a lung adenocarcinoma. In one embodiment, the cancer is chosen from a lung cancer, a pancreatic cancer, melanoma, a colorectal cancer, an esophageal-gastric cancer, a thyroid cancer, or an adenocarcinoma. In one embodiment, the cancer is a thyroid cancer. In one embodiment, the cancer is a papillary thyroid carcinoma. In one embodiment, the cancer is a lung adenocarcinoma. In other embodiment, the lung cancer is chosen from one or more of the following: non-small cell lung cancer (NSCLC), small cell lung cancer (SCLC), squamous cell carcinoma (SCC), adenocarcinoma of the lung, bronchogenic carcinoma, or a combination thereof. In one embodiment, the lung cancer is NSCLC or SCC.

In one embodiment, the anti-cancer agent is a RET inhibitor. In one embodiment, the anti-cancer agent is a LMNA inhibitor. In one embodiment, the anti-cancer agent is a kinase inhibitor. In one embodiment, the anti-cancer agent is a RET inhibitor, e.g., lestaurtinib (CEP-701); AZ-23; indenopyrrolocarboazole 12a; oxindole 3; isothiazole 5n; thiazole 20h.

### FMN1-ROS1 Fusions

In one embodiment, a fusion includes an in-frame fusion of an exon of formin 1 (FMN1), e.g., one more exons of FMN1 (e.g., one or more of exons 1-23 of FMN1) or a fragment thereof, and an exon of C-Ros oncogene 1 (ROS1), e.g., one or more exons of a ROS1 (e.g., one or more of exons 35-43 of ROS1) or a fragment thereof. For example, the FMN1-ROS1 fusion can include an in-frame fusion within an intron of FMN1 (e.g., intron 23) or a fragment thereof, with an intron of ROS1 (e.g., intron 34) or a fragment thereof. In one embodiment, the fusion of the FMN1-ROS1 fusion comprises the nucleotide sequence of: chromosome 15 at one or more of a nucleotide (plus or minus 10, 20, 30, 50, 60, 70, 80, 100 or more nucleotides) and chromosome 6 at one or more of a nucleotide (plus or minus 10, 20, 30, 50, 60, 70, 80, 100 or more nucleotides). In one embodiment, the FMN1-ROS1 fusion is a translocation, e.g., a translocation of a portion of chromosome 15 and a portion of chromosome 6.

In certain embodiments, the FMN1-ROS1 fusion is in a 5′-FMN1 to 3′-ROS1 configuration (also referred to herein as “5′-FMN1-ROS1-3′).” The term “fusion” or “fusion molecule” can refer to a polypeptide or a nucleic acid fusion, depending on the context. It may include a full-length sequence of a fusion or a fragment thereof, e.g., a fusion junction (e.g., a fragment including a portion of FMN1 and a portion of ROS1, e.g., a portion of the FMN1-ROS1 fusion described herein). In one embodiment, the FMN1-ROS1 fusion polypeptide includes a fragment of the amino acid sequence shown in SEQ ID NO:73 and a fragment of the amino acid sequence shown in SEQ ID NO: 12, or an amino acid sequence substantially identical thereto. In another embodiment, the FMN1-ROS1 fusion nucleic acid includes a fragment of the nucleotide sequence shown in SEQ ID NO:72 and a fragment of the nucleotide sequence shown in SEQ ID NO: 11, or a nucleotide sequence substantially identical thereto. In one embodiment, the FMN1-ROS1 fusion polypeptide comprises sufficient FMN1 and sufficient ROS1 sequence such that the 5′ FMN1-3′ ROS1 fusion has kinase activity, e.g., has elevated activity, e.g., tyrosine kinase activity. In any event, the fusion causes activation in the cells harboring it of oncogenic signaling pathways.

In certain embodiments, the FMN1-ROS1 fusion comprises one or more (or all of) exons 1-23 from FMN1 and one or more (or all of) exons 35-43 of ROS1 (e.g., one or more of the exons shown in SEQ ID NO:72 and SEQ ID NO: 11. In another embodiment, the FMN1-ROS1 fusion comprises one or more (or all of) exons 1-23 of FMN1 and one or more (or all of) exons 35-43 of ROS1. In certain embodiments, the FMN1-ROS1 fusion comprises at least 1, 2, 3, 4, 5, 6, 7, 8, 9, 10, 11, 12, 13, 14, 15, 16, 17, 18, 19, 20, 21, 22, 23 or more exons (or encoded exons) from FMN1 and at least 1, 2, 3, 4, 5, 6, 7, 8, 9 or more exons (or encoded exons) from ROS1 (e.g., from the FMN1 and ROS1 sequences shown in SEQ ID NO:72 and SEQ ID NO:73 and SEQ ID NO:11 and SEQ ID NO: 12.

In certain embodiments, the FMN1-ROS1 fusion comprises exons 1-23 or a fragment thereof from FMN1, and exons 35-43 or a fragment thereof from ROS1 (e.g., as shown in SEQ ID NO:72 and SEQ ID NO: 11). In one embodiment, the FMN1-ROS1 fusion comprises at least 5, 10, 15, 20, 30, 40, 50 or more amino acids from exons 1-23 of FMN1 (e.g., from the amino acid sequence of FMN1 as shown in SEQ ID NO:73 (e.g., from the amino acid sequence of FMN1 preceding the fusion junction with ROS1, and at least 5, 10, 15, 20, 30, 40, 50 or more amino acids from exons 35-43 of ROS1 (e.g., from the amino acid sequence of ROS1 as shown in SEQ ID NO: 12). In another embodiment, the FMN1-ROS1 fusion comprises at least 6, 12, 15, 20, 25, 50, 75, 100 or more nucleotides from exons 1-23 of FMN1 (e.g., from the nucleotide sequence of FMN1 as shown in 125 (SEQ ID NO:72) (e.g., from the nucleotide sequence of FMN1 preceding the fusion junction with ROS1); and at least 6, 12, 15, 20, 25, 50, 75, 100 or more nucleotides from exons 35-43 of ROS1 (e.g., from the nucleotide sequence of ROS1 as shown in 112 (SEQ ID NO:11)).

In one embodiment, the FMN1-ROS1 fusion includes the full sequence or a fragment of the nucleotide sequence shown in SEQ ID NO: 112, or a nucleotide sequence substantially identical thereto. In another embodiment, the FMN1-ROS1 fusion polypeptide includes the full sequence or a fragment of the amino acid sequence shown in SEQ ID NO: 113, or an amino acid sequence substantially identical thereto. The sequences SEQ ID NO: 112 and SEQ ID NO: 113 have been deduced from sequencing information by reference to NM_{—}001277313 for protein FMN1 and NM_{—}002944 for kinase ROS1.

### FMN1-ROS1 Nucleic Acid Molecules

In one aspect, the invention features a nucleic acid molecule (e.g., an isolated or purified) nucleic acid molecule that includes a fragment of a FMN1 gene and a fragment of a ROS1 gene. In one embodiment, the nucleotide sequence encodes a FMN1-ROS1 fusion polypeptide that includes a tyrosine kinase domain or a functional fragment thereof. In another embodiment, the nucleotide sequence encodes a fragment of the ROS1 polypeptide including the amino acid sequence of SEQ ID NO: 12 or a fragment thereof, or a sequence substantially identical thereto. In other embodiments, the nucleic acid molecule includes a fragment of the FMN1 gene encoding the amino acid sequence of SEQ ID NO:73 or a fragment thereof, or a sequence substantially identical thereto, for example 90% identical. In yet other embodiments, the nucleic acid molecule includes a nucleotide sequence encoding the amino acid sequence shown in 126 SEQ ID NO:73, or a fragment thereof, and the amino acid sequence shown in 113 SEQ ID NO:12 or a fragment thereof, or a sequence substantially identical thereto, for example a sequence at least 70%, or at least 80% or at least 90% or an even higher percentage identical.

In one embodiment, the nucleic acid molecule includes a fusion, e.g., an in-frame fusion, between an intron of FMN1 (e.g., intron 23, or a fragment thereof), and an intron of ROS1 (e.g., intron 34, or a fragment thereof). The FMN1-ROS1 fusion can comprise a fusion of the nucleotide sequence of one or more introns and/or exons with a nucleic acid encoding a protein partner on chromosome 15. The insertion of the nucleic acid encoding the kinase domain results in a fusion junction between the nucleic acid encoding the kinase domain or introns surrounding the kinase domain and a fusion junction between the nucleic acid encoding the kinase domain and the remainder of the chromosome (for example, at the 3′ end of the kinase domain). Examples of the locations of the junctions on chromosome 15 are set forth in additional disclosure Paragraphs JJ-LL. The fusion junctions can be found on chromosome 15 at one or more of a nucleotide (plus or minus 10, 20, 30, 50, 60, 70, 80, 100 nucleotides) and chromosome 6 at one or more of a nucleotide (plus or minus 10, 20, 30, 50, 60, 70, 80, 100 nucleotides), or a fragment thereof. In one embodiment, the HLA-A-ROS1 fusion comprises a fusion of the nucleotide sequence of: chromosome 15 at one or more of a nucleotide (plus or minus 10, 20, 30, 50, 60, 70, 80, 100 nucleotides) and chromosome 6 at one or more of a nucleotide (plus or minus 10, 20, 30, 50, 60, 70, 80, 100 nucleotides), or a fragment thereof.

In another embodiment, the FMN1-ROS1 fusion comprises a nucleotide sequence (e.g., a fragment of a nucleotide sequence) shown in SEQ ID NO:72 and a nucleotide sequence (e.g., a fragment of a nucleotide sequence) shown in SEQ ID NO:11, or a fragment of the fusion. In one embodiment, the FMN1-ROS1 fusion comprises a nucleotide sequence substantially identical to the nucleotide sequence (e.g., a fragment of a nucleotide sequence) shown in SEQ ID NO:72 and the nucleotide sequence (e.g., a fragment of a nucleotide sequence) shown SEQ ID NO: 11, or a fragment of the fusion. In one embodiment, the FMN1-ROS1 fusion comprises a nucleotide sequence at least 70%, at least 75%, at least 80%, at least 85%, at least 90%, at least 95%, at least 99%, at least 99.5 or greater, identical to the nucleotide sequence (e.g., a fragment of a nucleotide sequence) shown in SEQ ID NO:72 and to the nucleotide sequence (e.g., a fragment of a nucleotide sequence) shown in SEQ ID NO: 1. In one embodiment, the FMN1-ROS1 fusion comprises a nucleotide sequence containing at least 25, 50, 100, 150, 200, 500, 1000, 1500, 2000, 2500, 3000, or more nucleotides of the nucleotide sequence shown in SEQ ID NO:72 and a nucleotide sequence containing at least 25, 50, 100, 150, 200, 500, 1000, 1500, 2000, 2500, 3000, or more nucleotides of the nucleotide sequence shown in SEQ ID NO:11. In one embodiment, the FMN1-ROS1 fusion comprises a nucleotide sequence containing at least 25, 50, 100, 150, 200, 500, 1000, 1500, 2000, 2500, 3000, or more contiguous nucleotides of the nucleotide sequence shown in SEQ ID NO:72 and a nucleotide sequence containing at least 25, 50, 100, 150, 200, 500, 1000, 1500, 2000, 2500, 3000, or more nucleotides of the nucleotide sequence shown in SEQ ID NO: 11.

In another embodiment, the nucleic acid molecule includes a fusion, e.g., an in-frame fusion, of at least 1, 2, 3, 4, 5, 6, 7, 8, 9, 10, 11, 12, 13, 14, 15, 16, 17, 18, 19, 20, 21, 22, 23 or more exons of FMN1 or a fragment thereof (e.g., one or more of exons 1-23 of FMN1 or a fragment thereof), and at least 1, 2, 3, 4, 5, 6, 7, 8, 9 or more exons of ROS1 or a fragment thereof (e.g., one or more of exons 35-43 of ROS1 or a fragment thereof). In yet other embodiments, the nucleic acid molecule includes a fragment the nucleotide sequence shown in SEQ ID NO:72 and a fragment of the nucleotide sequence shown in SEQ ID NO:11 or a fragment of the fusion, or a sequence substantially identical thereto.

In one embodiment, the nucleic acid molecule is complementary to at least a portion of a nucleotide sequence disclosed herein, e.g., is capable of hybridizing under a stringency condition described herein to SEQ ID NO:72 and/or SEQ ID NO: 11, or a fragment thereof. In yet another embodiment, the nucleic acid molecule hybridizes to a nucleotide sequence that is complementary to at least a portion of a nucleotide sequence disclosed herein, e.g., is capable of hybridizing under a stringency condition to a nucleotide sequence complementary to SEQ ID NO:72 and/or SEQ ID NO: 11, or a fragment thereof. The nucleotide sequence of a cDNA encoding an exemplary 5′ FMN1-3′ ROS1 fusion is shown in at least exon 23 (e.g., exons 1-23) of SEQ ID NO:72 and at least exon 35 (e.g., exons 35-43) of SEQ ID NO: 11, and the predicted amino acid sequence is shown in the corresponding encoded exons of SEQ ID NO:73 and the corresponding encoded exons of SEQ ID NO: 12, respectively.

In an embodiment the FMN1-ROS1 nucleic acid molecule comprises sufficient FMN1 and sufficient ROS1 sequence such that the encoded 5′ FMN1-3′ ROS1 fusion has kinase activity, e.g., has elevated activity. In certain embodiments, the 5′ FMN1-3′ ROS1 fusion comprises exons 1-23 from FMN1 and exons 35-43 from ROS1. In certain embodiments, the FMN1-ROS1 fusion comprises at least 1, 2, 3, 4, 5, 6, 7, 8, 9, 10, 11, 12, 13, 14, 15, 16, 17, 18, 19, 20, 21, 22, 23 or more exons from FMN1 and at least at least 1, 2, 3, 4, 5, 6, 7, 8, or more exons of ROS1. In certain embodiments, the FMN1-ROS1 fusion comprises a fusion of exon 23 from FMN1 and exon 35 from ROS1. In another embodiment, the FMN1-ROS1 fusion comprises 1, 2, 3, 4, 5, 6, 7, 8, 9, 10, 11, 12, 13, 14, 15, 16, 17, 18, 19, 20, 21, 22, 23 or more exons of FMN1; and at least 1, 2, 3, 4, 5, 6, 7, 8, 9 or more exons of ROS1.

In one embodiment, the nucleic acid molecule includes a nucleotide sequence that has an in-frame fusion of intron 23 of FMN1 (e.g., NM_{—}000259) with intron 34 of ROS1 (e.g., NM_{—}002944). In another embodiment, the nucleic acid molecule includes a nucleotide sequence that includes a breakpoint. For example, the nucleic acid molecule includes a nucleotide sequence that includes the fusion junction between the FMN1 gene and the ROS1 gene, e.g., the breakpoint between intron 23 of FMN1 and intron 34 of ROS1. In other embodiments, the nucleic acid molecules includes a nucleotide sequence of one or more of nucleotide of chromosome 15 coupled to (e.g., directly or indirectly juxtaposed to) one or more of nucleotide of chromosome 6. In one embodiment, the nucleic acid molecule includes the nucleotide sequence of: chromosome 15 at one or more of a nucleotide plus or minus 10, 20, 30, 40 50, 60, 80, 100, 150 nucleotides and chromosome 6 at one or more of a nucleotide plus or minus 10, 20, 30, 40 50, 60, 80, 100, 150 nucleotides (corresponding to the breakpoint of a FMN1-ROS1 fusion), or a fragment thereof, or a sequence substantially identical thereto. In one embodiment, the nucleic acid molecule is complementary to at least a portion of a nucleotide sequence disclosed herein, e.g., is capable of hybridizing under a stringency condition described herein to SEQ ID NO:72 and/or SEQ ID NO: 11 or a fragment thereof. In yet other embodiments, the nucleic acid molecule hybridizes to a nucleotide sequence that is complementary to at least a portion of a nucleotide sequence disclosed herein, e.g., is capable of hybridizing under a stringency condition described herein to a nucleotide sequence complementary to SEQ ID NO:72 or SEQ ID NO: 11 or a fragment thereof.

In another embodiment, the FMN1-ROS1 fusion nucleic acid comprises at least 6, 12, 15, 20, 25, 50, 75, 100 or more nucleotides from exon 23 of FMN1 (e.g., from the nucleotide sequence of FMN1 preceding the fusion junction with ROS1, e.g., of the FMN1 sequence shown in SEQ ID NO:72), and at least 6, 12, 15, 20, 25, 50, 75, 100 or more nucleotides from exon 35 of ROS1 (e.g., from the nucleotide sequence of ROS1 following the fusion junction with FMN1, e.g., of the ROS1 sequence shown in SEQ ID NO:11).

In other embodiments, the nucleic acid molecule includes a nucleotide sequence encoding a FMN1-ROS1 fusion polypeptide that includes a fragment of a FMN1 gene and a fragment of a ROS1 gene. In one embodiment, the nucleotide sequence encodes a FMN1-ROS1 fusion polypeptide that includes e.g., a tyrosine kinase domain or a functional fragment thereof. In yet other embodiments, the nucleic acid molecule includes a nucleotide sequence encoding the amino acid sequence (e.g., a fragment of the amino acid sequence) shown in SEQ ID NO:73 and a nucleotide sequence encoding the amino acid sequence (e.g., a fragment of the amino acid sequence) shown in SEQ ID NO: 12, or a fragment of the fusion, or a sequence substantially identical thereto. In one embodiment, the encoded FMN1-ROS1 fusion polypeptide includes a tyrosine kinase domain or a functional fragment thereof.

In a related aspect, the invention features nucleic acid constructs that include the FMN1-ROS nucleic acid molecules described herein. In certain embodiments, the nucleic acid molecules are operatively linked to a native or a heterologous regulatory sequence. Also included are vectors and host cells that include the FMN1-ROS1 nucleic acid molecules described herein, e.g., vectors and host cells suitable for producing the nucleic acid molecules and polypeptides described herein.

In a related aspect, methods of producing the nucleic acid molecules and polypeptides described herein are also described.

In another aspect, the invention features nucleic acid molecules that reduce or inhibit the expression of a nucleic acid molecule that encodes a FMN1-ROS1 fusion described herein. Examples of such nucleic acid molecules include, for example, antisense molecules, ribozymes, RNAi, triple helix molecules that hybridize to a nucleic acid encoding FMN1-ROS1, or a transcription regulatory region of FMN1-ROS1, and blocks or reduces mRNA expression of FMN1-ROS.

### Nucleic Acid Detection and Capturing Reagents

The invention also features a nucleic acid molecule, e.g., nucleic acid fragment, suitable as probe, primer, bait or library member that includes, flanks, or hybridizes to the FMN1-ROS1 fusions described herein. Such nucleic acid molecules are useful for identifying, or are otherwise based on, the FMN1-ROS1 fusions described herein. In certain embodiments, the probe, primer or bait molecule is an oligonucleotide that allows capture, detection or isolation of a FMN1-ROS1 fusion nucleic acid molecule described herein. The oligonucleotide can comprise a nucleotide sequence substantially complementary to a fragment of the FMN1-ROS1 fusion nucleic acid molecules described herein. The sequence identity between the nucleic acid fragment, e.g., the oligonucleotide, and the target FMN1-ROS1 sequence need not be exact, so long as the sequences are sufficiently complementary to allow the capture, detection or isolation of the target sequence. In one embodiment, the nucleic acid fragment is a probe or primer that includes an oligonucleotide between about 5 and 25, e.g., between 10 and 20, or 10 and 15 nucleotides in length. In other embodiments, the nucleic acid fragment is a bait that includes an oligonucleotide between about 100 to 300 nucleotides, 130 and 230 nucleotides, or 150 and 200 nucleotides, in length.

In one embodiment, the nucleic acid fragment can be used to identify or capture. e.g., by hybridization, a FMN1-ROS1 fusion. For example, the nucleic acid fragment can be a probe, a primer, or a bait, for use in identifying or capturing, e.g., by hybridization, a FMN1-ROS1 fusion described herein. In one embodiment, the nucleic acid fragment can be useful for identifying or capturing a FMN1-ROS1 breakpoint, e.g., the nucleotide sequence of: chromosome 15 at nucleotide plus or minus 10, 20, 30, 40, 50, 60, 80, 100, 150 nucleotides and chromosome 6 at nucleotide plus or minus 10, 20, 30, 40 50, 60, 80, 100, 150 nucleotides.

In one embodiment, the nucleic acid fragment hybridizes to a nucleotide sequence within a chromosomal rearrangement that creates an in-frame fusion of intron 23 of FMN1 with intron 34 of ROS1. In one embodiment, the nucleic acid fragment hybridizes to a nucleotide sequence in the region In other embodiments, the nucleic acid molecules includes a nucleotide sequence in the region of a nucleotide sequence of chromosome 15 coupled to (e.g., juxtaposed to) nucleotides in the region of a nucleotide sequence Y of chromosome 6. In one embodiment, the nucleic acid fragment hybridizes to a nucleotide sequence that includes a breakpoint, e.g., the nucleotide sequence of: chromosome 15 at nucleotide plus or minus 10, 20, 30, 40 50, 60, 80, 100, 150 or more nucleotides and chromosome 6 at nucleotide plus or minus 10, 20, 30, 40 50, 60, 80, 100, 150 or more nucleotides. For example, the nucleic acid fragment can hybridize to a nucleotide sequence that includes the fusion junction between the FMN11 gene and the ROS1 gene, e.g., a nucleotide sequence that includes a portion of a nucleotide sequence within intron 23 of a FMN1 gene and intron 34 of a ROS1 gene.

In another embodiment, the nucleic acid fragment hybridizes to a nucleotide sequence that comprises at least 6, 12, 15, 20, 25, 50, 75, 100, 150 or more nucleotides from exon 23 of FMN1 (e.g., from the nucleotide sequence of FMN1 preceding the fusion junction with ROS1, e.g., of the FMN1 sequence shown in SEQ ID NO:72), and at least 6, 12, 15, 20, 25, 50, 75, 100, 150 or more nucleotides from exon 35 of ROS1 (e.g., from the nucleotide sequence of ROS1 following the fusion junction with FMN1, e.g., of the ROS1 sequence shown in SEQ ID NO: 11).

The probes or primers described herein can be used, for example, for FISH detection or PCR amplification. In one exemplary embodiment where detection is based on PCR, amplification of the FMN1-ROS1 fusion junction fusion junction can be performed using a primer or a primer pair, e.g., for amplifying a sequence flanking the fusion junctions described herein, e.g., the mutations or the junction of a chromosomal rearrangement described herein, e.g., FMN1-ROS1.

In one embodiment, a pair of isolated oligonucleotide primers can amplify a region containing or adjacent to a position in the FMN1-ROS1 fusion. For example, forward primers can be designed to hybridize to a nucleotide sequence within FMN1 genomic or mRNA sequence (e.g., a nucleotide sequence within exon 23 of FMN1 of SEQ ID NO:72), and the reverse primers can be designed to hybridize to a nucleotide sequence of ROS1 (e.g., a nucleotide sequence within exon 35 of ROS1, of SEQ ID NO: 11).

In another embodiment, the nucleic acid fragments can be used to identify, e.g., by hybridization, a FMN1-ROS1 fusion. In one embodiment, the nucleic acid fragment hybridizes to a nucleotide sequence that includes a fusion junction between the FMN1 transcript and the ROS1 transcript.

In other embodiments, the nucleic acid fragment includes a bait that comprises a nucleotide sequence that hybridizes to a FMN1-ROS1 fusion nucleic acid molecule described herein, and thereby allows the capture or isolation said nucleic acid molecule. In one embodiment, a bait is suitable for solution phase hybridization. In other embodiments, a bait includes a binding entity, e.g., an affinity tag, that allows capture and separation, e.g., by binding to a binding entity, of a hybrid formed by a bait and a nucleic acid hybridized to the bait.

In other embodiments, the nucleic acid fragment includes a library member comprising a FMN1-ROS1 nucleic acid molecule described herein. In one embodiment, the library member includes a rearrangement that results in a FMN1-ROS1 fusion described herein.

The nucleic acid fragment can be detectably labeled with, e.g., a radiolabel, a fluorescent label, a bioluminescent label, a chemiluminescent label, an enzyme label, a binding pair label, or can include an affinity tag; a tag, or identifier (e.g., an adaptor, barcode or other sequence identifier).

### FMN1-ROS1 Fusion Polypeptides

In another embodiment, the FMN1-ROS1 fusion comprises an amino acid sequence (e.g., a fragment of the amino acid sequence) shown in SEQ ID NO:73 and an amino acid sequence (e.g., a fragment of the amino acid sequence) shown in SEQ ID NO: 12, or a fragment of the fusion. In one embodiment, the FMN1-ROS1 fusion comprises an amino acid sequence substantially identical to the amino acid sequence (e.g., a fragment of the amino acid sequence) shown in SEQ ID NO:73 and the amino acid sequence (e.g., a fragment of the amino acid sequence) shown in SEQ ID NO:12, or a fragment thereof. In one embodiment, the FMN1-ROS1 fusion comprises an amino acid sequence at least 70%, at least 75%, at least 80%, at least 85%, at least 90%, at least 95%, at least 99%, at least 99.5 or greater, identical to the amino acid sequence (e.g., a fragment of the amino acid sequence) shown in SEQ ID NO:73 and the amino acid sequence (e.g., a fragment of the amino acid sequence) shown in SEQ ID NO:12. In one embodiment, the FMN1-ROS1 fusion comprises a sequence containing at least 10, 20, 50, 100, 500, 600, 700, 800, 900, 1000, or more amino acids of the amino acid sequence shown in SEQ ID NO:73 and SEQ ID NO: 12. In one embodiment, the FMN1-ROS1 fusion comprises an amino acid sequence containing at least 5, 10, 20, 50, 100, 500, 600, 700, 800, 900, 1000, or more contiguous amino acids of the amino acid sequence shown in SEQ ID NO:73 and at least 5, 10, 20, 50, 100, 500, 600, 700, 800, 900, 1000, or more contiguous amino acids of the amino acid sequence shown in SEQ ID NO:12. In one embodiment, the 5′ FMN1-3′ ROS1 fusion polypeptide includes a receptor tyrosine kinase domain or a functional fragment thereof. In an embodiment, the 5′FMN1-3′ROS1 fusion polypeptide comprises sufficient ROS1 and sufficient FMN1 sequence such that it has kinase activity, e.g., has elevated activity.

In another aspect, the invention features a FMN1-ROS1 fusion polypeptide (e.g., a purified FMN1-ROS1 fusion polypeptide), a biologically active or antigenic fragment thereof, as well as reagents (e.g., antibody molecules that bind to a FMN1-ROS1 fusion polypeptide), methods for modulating a FMN1-ROS1 polypeptide activity and detection of a FMN1-ROS1 polypeptide.

In one embodiment, the FMN1-ROS1 fusion polypeptide has at least one biological activity. In one embodiment, at least one biological activity of the FMN1-ROS1 fusion polypeptide is reduced or inhibited by an anti-cancer drug, e.g., a FMN1 inhibitor, a ROS1 inhibitor. In one embodiment, at least one biological activity of the FMN1-ROS1 fusion polypeptide is reduced or inhibited by a ROS1 inhibitor. In one embodiment, at least one biological activity of the FMN1-ROS1 fusion polypeptide is reduced or inhibited by a FMN1 inhibitor. In one embodiment, at least one biological activity of the FMN1-ROS1 fusion polypeptide is reduced or inhibited by a ROS1 inhibitor. e.g., Ganetespib; Crizotinib; TAE684, a dual ALK and ROS1 inhibitor.

In yet other embodiments, the FMN1-ROS1 fusion polypeptide is encoded by a nucleic acid molecule described herein. In one embodiment, the FMN1-ROS1 fusion polypeptide is encoded by an in-frame fusion of intron 23 of FMN1 with intron 34 of ROS1 (e.g., a sequence on chromosome 15 and a sequence on chromosome 6). In another embodiment, the FMN1-ROS1 fusion polypeptide includes an amino acid sequence encoded by a nucleotide sequence comprising a fusion junction between the FMN1 transcript and the ROS1 transcript.

In certain embodiments, the FMN1-ROS1 fusion polypeptide comprises one or more of encoded exons 1-23 from FMN1 and one or more of encoded exons 35-43 of ROS1. In certain embodiments, the FMN1-ROS1 fusion polypeptide comprises at least 1, 2, 3, 4, 5, 6, 7, 8, 9, 10, 11, 12, 13, 14, 15, 16, 17, 18, 19, 20, 21, 22, 23 or more encoded exons of FMN1 and at least at least 1, 2, 3, 4, 5, 6, 7, 8, 9 or more encoded exons of ROS1. In certain embodiments, the FMN1-ROS1 fusion polypeptide comprises a fusion of encoded exon 23 from FMN1 and encoded exon 35 from ROS1 (or a fragment thereof). In other embodiments, the fusion comprises at least 1, 2, 3, 4, 5, 6, 7, 8, 9, 10, 11, 12, 13, 14, 15, 16, 17, 18, 19, 20, 21, 22, 23 or more encoded exons of FMN1; and at least 1, 2, 3, 4, 5, 6, 7, 8, 9 or more encoded exons of ROS1. In certain embodiments, the FMN1-ROS1 fusion polypeptide comprises encoded exons 1-23 from FMN1 and exons 35-43 of ROS1. In certain embodiments, the 5′ FMN1-3′ ROS1 fusion polypeptide comprises a fusion junction of the sequence of exon 23 from FMN1 and the sequence of exon 35 from ROS1.

In certain embodiments, the FMN1-ROS1 fusion comprises the amino acid sequence corresponding to exon 23 or a fragment thereof from FMN1, and the amino acid sequence corresponding to exon 35 or a fragment thereof from ROS1 (e.g., as shown in SEQ ID NO:73 and SEQ ID NO:12). In one embodiment, the FMN1-ROS1 fusion comprises at least 5, 10, 15, 20 or more amino acids from exon 23 of FMN1 (e.g., from the amino acid sequence of FMN1 preceding the fusion junction with ROS1, e.g., of the FMN1 sequence shown in SEQ ID NO:73), and at least 5, 10, 15, 20 or more amino acids from exon 35 of ROS1 (e.g., from the amino acid sequence of ROS1 following the fusion junction with FMN1, e.g., of the ROS1 sequence shown in SEQ ID NO: 12).

In one embodiment, the FMN1-ROS1 fusion polypeptide includes a tyrosine kinase domain or a functional fragment thereof. In a related aspect, the invention features FMN1-ROS1 fusion polypeptide or fragments operatively linked to heterologous polypeptides to form fusion proteins.

In another embodiment, the FMN1-ROS1 fusion polypeptide or fragment is a peptide, e.g., an immunogenic peptide or protein containing a fusion junction described herein. Such immunogenic peptides or proteins can be used to raise antibodies specific to the fusion protein. In other embodiments, such immunogenic peptides or proteins can be used for vaccine preparation. The vaccine preparation can include other components, e.g., an adjuvant.

In another aspect, the invention features antibody molecules that bind to a FMN1-ROS1 fusion polypeptide or fragment described herein. In embodiments, the antibody can distinguish wild type ROS1 (or FMN1) from FMN1-ROS1.

### Detection Reagents and Detection of Mutations

In another aspect, the invention features a detection reagent, e.g., a purified or an isolated preparation thereof. Detection reagents can distinguish a nucleic acid, or protein sequence, having a breakpoint, e.g., a FMN1-ROS1 breakpoint; from a reference sequence. In one embodiment, the detection reagent detects (e.g., specifically detects) a FMN1-ROS1 fusion nucleic acid or a polypeptide, e.g., distinguishes a wild type ROS1 or another ROS1 fusion (or FMN1) from a FMN1-ROS1 nucleic acid (e.g., as described herein in SEQ ID NO:72 and SEQ ID NO: 11); or a FMN1-ROS1 polypeptide (e.g., as described herein in SEQ ID NO:73 and SEQ ID NO:12).

Detection reagents, e.g., nucleic acid-based detection reagents, can be used to identify mutations in a target nucleic acid, e.g., DNA, e.g., genomic DNA or cDNA, or RNA, e.g., in a sample, e.g., a sample of nucleic acid derived from a neoplastic or tumor cell, e.g., a melanocytic neoplasm, melanoma or metastatic cell. Detection reagents, e.g., antibody-based detection reagents, can be used to identify mutations in a target protein, e.g., in a sample, e.g., a sample of protein derived from, or produced by, a neoplastic or tumor cell, e.g., a melanocytic neoplasm, melanoma or metastatic cell.

### Method of Treatment

In a related aspect, a method of inhibiting, reducing, or treating a hyperproliferative disorder, e.g., a neoplasm (including benign, pre-malignant or malignant (e.g., a cancer), in a subject is provided. The method includes administering to the subject a preselected therapeutic agent, e.g., an anti-cancer agent (e.g., a kinase inhibitor), as a single agent, or in combination, in an amount sufficient to reduce, inhibit or treat the activity or expression of FMN1-ROS1 (e.g., a FMN1-ROS1 fusion described herein), thereby inhibiting, reducing, or treating the hyperproliferative disorder in the subject.

In one embodiment, the subject treated has a FMN1-ROS1 fusion; e.g., the subject has a tumor or cancer harboring a FMN1-ROS1 fusion. In other embodiments, the subject has been previously identified as having a FMN1-ROS1 fusion. In yet other embodiments, the subject has been previously identified as being likely or unlikely to respond to treatment with a protein kinase inhibitor, e.g., a subject that has previously participated in a clinical trial. In other embodiments, the subject has been previously identified as being likely or unlikely to respond to treatment with a protein kinase inhibitor, based on the presence of the FMN1-ROS1 fusion. In one embodiment, the subject is a mammal, e.g., a human. In one embodiment, the subject has, or is at risk of having a cancer at any stage of disease. In other embodiments, the subject is a patient, e.g., a cancer patient.

In certain embodiments, the neoplasm or neoplastic cell is a benign, pre-malignant, malignant (cancer) or metastasis. In certain embodiments, the cancer is a solid tumor, a soft tissue tumor, or a metastatic lesion.

In one embodiment, the neoplasm or neoplastic cell is a benign, pre-malignant, malignant (cancer) or metastasis. In certain embodiments, the cancer is a solid tumor, a soft tissue tumor, or a metastatic lesion. In one embodiment, the cancer is a carcinoma. In one embodiment, the cancer is an adenocarcinoma. In an embodiment, the cancer is a lung adenocarcinoma. In one embodiment, the cancer is chosen from a lung cancer, a pancreatic cancer, melanoma, a colorectal cancer, an esophageal-gastric cancer, a thyroid cancer, or an adenocarcinoma. In one embodiment, the cancer is a lung adenocarcinoma. In other embodiment, the lung cancer is chosen from one or more of the following: non-small cell lung cancer (NSCLC), small cell lung cancer (SCLC), squamous cell carcinoma (SCC), adenocarcinoma of the lung, bronchogenic carcinoma, or a combination thereof. In one embodiment, the lung cancer is NSCLC or SCC.

In one embodiment, the anti-cancer agent is a ROS1 inhibitor. In one embodiment, the anti-cancer agent is a FMN1 inhibitor. In one embodiment, the anti-cancer agent is a kinase inhibitor. In one embodiment, the anti-cancer agent is a ROS1 inhibitor, e.g., Ganetespib; Crizotinib; TAE684; a dual ALK and ROS1 inhibitor.

### Nucleic Acid Molecules

In one aspect, the invention features, an isolated nucleic acid molecule, or an isolated preparation of nucleic acid molecules, that includes a genetic alteration or mutation, e.g., a rearrangement, disclosed herein, e.g., in this section entitled Nucleic Acid Molecules, or in FIG. 1A. 1B or 1C. Such nucleic acid molecules or preparations thereof can be used to detect, e.g., sequence, a genetic alteration or mutation disclosed herein and to characterize a sample in which they are contained. The isolated nucleic acid can be a genomic or a transcribed sequence, e.g., cDNA or mRNA sequence.

In another aspect, the invention features, a nucleic acid molecule (e.g., an isolated or purified) nucleic acid molecule that includes a fragment of a first gene, and a fragment of a second gene, the latter typically a gene that encodes a kinase. In embodiments, the first gene is a gene from FIG. 1A, 1B or 1C and the second gene is a gene, e.g., a kinase from FIG. 1A, 1B or 1C. In an embodiment the fusion protein has the fusion partners of a fusion protein described in FIG. 1A, 1B or 1C.

The isolated nucleic acid molecule can comprise the entire sequence of the first fragment and the entire sequence of the second fragment, e.g., as shown in FIG. 1A, 1B or 1C.

In embodiments the isolated nucleic acid is a genomic nucleic acid molecule comprises sequence encoding the entire sequence, e.g., from the control region or beginning of the open reading frame, through the breakpoint, which may be in an intron or an exon, of the first gene, fused to the a sequence for the second gene which begins at its breakpoint and extends to the end of the gene, e.g., through the end of the open reading frame of that gene. In other embodiments the isolated nucleic acid will include the fusion junction but only a portion of the fragment of the first or second gene present in the rearrangement.

In embodiments the isolated nucleic acid is a transcribed nucleic acid, e.g., a cDNA or mRNA, and comprises sequence encoding the entire sequence, e.g., from the beginning of the mRNA through the breakpoint of the first gene fused to the a sequence for the second gene which begins at its breakpoint and extends to the end of the mRNA of the second gene. In other embodiments the isolated nucleic acid will include the fusion junction but only a portion of the fragment of the first or second gene present in the rearrangement. In embodiments a transcribed nucleic acid will have one or more exon from the first gene fused, in frame, to one or more exons of the second or “host” gene. In embodiments a transcribed nucleic acid will have comprise the fusion of the C terminus of the C terminal exon of the first gene fragment with the N terminus of the N terminal exon of the second gene.

In embodiments the fusion puts the kinase activity of the second gene under the control of the first gene.

In embodiments the isolated nucleic acid, e.g., a genomic or transcribed nucleic acid, e.g., a cDNA or RNA, comprises the fusion junction, e.g., a fusion junction from FIG. 1A, 1B or 1C, and is at least 10, 20, 30, 40, 50, 60, 70, 80, 100, 125, 150, 200, 250, 300, 350, or 400 nucleotides in length, but optionally less than 1,000, 1,500, or 2,000 nucleotides in length. In embodiments, the isolated nucleic acid, e.g., a genomic or transcribed nucleic acid, e.g., a cDNA or RNA, comprises the fusion junction, e.g., a fusion junction from FIG. 1A, 1B or 1C, and is between 10 and 2,000, 10 and 1,500, 10 and 1,000, 10 and 500, 10 and 400, 10 and 300, 10 and 200, 10 and 100, 20 and 2,000, 20 and 1,500, 20 and 1,000, 20 and 500, 20 and 400, 20 and 300, 20 and 200, 20 and 100, 30 and 2,000, 30 and 1,500, 30 and 1,000, 30 and 500, 30 and 400, 30 and 300, 30 and 200, 30 and 100 nucleotides in length.

In one embodiment, the isolated nucleic acid, e.g., a transcribed nucleic acid, e.g., a cDNA or RNA, comprises a fusion, e.g., an in-frame fusion, from FIG. 1B or a fusion transcribed from a genomic fusion from FIG. 1A.

In an embodiment, the isolated nucleic acid, e.g., a transcribed nucleic acid, e.g., a cDNA or RNA, comprises a fusion, e.g., an in-frame fusion, of the 3′ terminus of an exon of a fragment of the first gene of FIG. 1B to the 5′ terminus of an exon of a fragment of the second gene of FIG. 1B. In an embodiment the fusion is between the exons listed in FIG. 1B. In embodiments, fusion is not be between the specific exons found in FIG. 1B but is between other exons of the first gene to other exons of the second gene of a fusion from FIG. 1B.

In an embodiment, the isolated nucleic acid, e.g., a transcribed nucleic acid, e.g., a cDNA or RNA, comprises a fusion, e.g., an in-frame fusion, of the C terminal exon of a fragment of first gene of FIG. 1B to the N terminus of an exon a fragment of the second gene other than the second gene exon shown in FIG. 1B. By way of example, an exon, e.g., exon 16 of CEP89 is fused to an exon, of BRAF other than the exon listed in FIG. X1, e.g., it is fused to an exon other than exon 9.

In an embodiment, the isolated nucleic acid, e.g., a transcribed nucleic acid, e.g., a cDNA or RNA, comprises a fusion, e.g., an in-frame fusion, of the N terminal exon of a fragment of the second gene of FIG. 1B to the C terminus of an exon of a fragment of the first gene other than the first-gene exon shown in FIG. 1B. By way of example, exon 9 of BRAF is fused to an exon of CEP89 other than the exon listed in FIG. 1B (exon 16)

In an embodiment of the isolated nucleic acid, e.g., a genomic or transcribed nucleic acid, e.g., a cDNA or RNA, the second gene is a kinase and sufficient exonic sequence is present to confer kinase activity. In an embodiment of the isolated nucleic acid, e.g., a genomic or transcribed nucleic acid, e.g., a cDNA or mRNA, sufficient sequence of the first gene is present to allow expression of kinase activity of the fusion partner.

In an embodiment of the isolated nucleic acid, e.g., a transcribed nucleic acid, e.g., a cDNA or RNA, comprises a fusion junction between:

CLIP1 and ROS1;

PPFIBP1 and ROS1;

TPM3 and ROS1;

ZCCHC8 and ROS1;

MYO5A and ROS1;

PWWP2A and ROS1;

HLA-A and ROS1;

ERC1 and ROS1;

TPM3 and ALK;

GOLGA5 and RET;

CEP89 and BRAF;

KIF5B and RET;

TP53 and NTRK1;

KIAA1598 and ROS1:

DCTN1 and ALK;

LSM14A and BRAF; or

LMNA and NTRK1.

wherein sufficient exonic sequence from the kinase is present to confer kinase activity and sufficient sequence of the other gene is present to allow expression of kinase activity of the fusion partner, in some embodiments rendering the kinase portion constitutively active and in any event enhancing its activity compared to the same kinase activity of either wild type gene.

Also included are genomic fusion that can be transcribed to provide a transcribed nucleic acid, e.g., a cDNA or RNA, described herein.

In one embodiment, the isolated nucleic acid, e.g., a genomic nucleic acid, comprises a fusion of a first and second gene from FIG. 1A.

In embodiments, the fusion is between genes that are fusion partners in a fusion described in FIG. 1A or 1B. In an embodiment sufficient sequence from the second gene is present to confer kinase activity on an encoded protein and sufficient sequence is present from the first gene to provide for expression of the kinase activity of the fusion partner in an encoded protein.

In an embodiment, the isolated nucleic acid, e.g., a genomic sequence, comprises a fusion of the 3′ terminus of a fragment of a first gene to the 5′ terminus of a fragment of a second gene, shown in FIG. 1A. In an embodiment, the 3′ terminus of the fragment of the first gene is within 10, 20, 30, 40, 50 60, 70, 80, 90, or 100 nucleotides (in either direction) of the 3-terminus provided in FIG. 1A for the first gene. In an embodiment, the 5′ terminus of the fragment of the second gene is within 10, 20, 30, 40, 50 60, 70, 80, 90, or 100 nucleotides (in either direction) of the 5′ terminus provided in FIG. 1 for the second gene. By way of example, for the CEP89-BRAF fusion from a cancer sample, the 3′ terminus can be a nucleotide sequence of chr19, +/−N nucleotides and the 5′ terminus is a nucleotide sequence of chr7, wherein N, independently is 10, 20, 30, 40, 50, 60, 70, 80, 90, or 100 nucleotides. In embodiments, N is 50 nucleotides.

The fusion need not be between the specific exons found in FIG. 1A or 1B but can be fusions of other exons of the first gene to other exons of the second gene, provided that sufficient sequence from the second gene is present to confer kinase activity on an encoded protein and sufficient sequence is present from the first gene to provide for expression of the kinase activity of the fusion partner in an encoded protein.

In another aspect, methods of producing the nucleic acid molecules and polypeptides described herein are also described.

### Detection Reagents and Detection of Mutations

In another aspect, the invention features a detection reagent, e.g., a purified or an isolated preparation thereof. Detection reagents can distinguish a nucleic acid, e.g., a genomic or transcribed nucleic acid, e.g., a cDNA or RNA, or protein sequence, having a breakpoint or fusion junction described herein, e.g., in FIG. 1A or 1B, or in the section herein entitled Nucleic Acid Molecules, from a reference sequence, e.g., a sequence not having the breakpoint or fusion junction.

In one embodiment, the detection reagent detects (e.g., specifically detects) a fusion nucleic acid or a polypeptide, e.g., distinguishes a wild type or another fusion from a fusion described herein, e.g., in FIG. 1A or 1B or in the section herein entitled Nucleic Acid Molecules.

Detection reagents, e.g., nucleic acid-based detection reagents, can be used to identify mutations, e.g., rearrangements or fusion junctions described herein, e.g., in FIG. 1A or 1B or in the section herein entitled Nucleic Acid Molecules, in a target nucleic acid, e.g., DNA, e.g., genomic DNA or a transcribed nucleic acid, cDNA, or RNA, e.g., in a sample, e.g., a sample of nucleic acid derived from a neoplastic or tumor cell, e.g., a primary or metastatic cell. In an embodiment a rearrangement or fusion junction described in FIG. 1A or 1B or in the section herein entitled Nucleic Acid Molecules, is detected in a sample of the corresponding cancer listed in FIG. 1A. Detection reagents, e.g., antibody-based detection reagents, can be used to identify, mutations described herein, e.g., in FIG. 1A or 1B or in the section herein entitled Nucleic Acid Molecules, in a target protein, e.g., in a sample, e.g., a sample of protein derived from, or produced by, a primary or metastatic cell.

### Nucleic Acid-Based Detection Reagents

In an embodiment, the detection reagent comprises a nucleic acid molecule, e.g., a DNA, RNA or mixed DNA/RNA molecule, comprising sequence which is complementary with a nucleic acid sequence on a target nucleic acid, e.g., a nucleic acid that includes the rearrangement or fusion junction, (the sequence on the target nucleic acid that is bound by the detection reagent is referred to herein as the “detection reagent binding site” and the portion of the detection reagent that corresponds to the detection reagent binding site is referred to as the “target binding site”). In an embodiment, the detection reagent binding site is disposed in relationship to the interrogation position, e.g., one or both nucleotides flanking the fusion junction, such that binding (or in embodiments, lack of binding) of the detection reagent to the detection reagent binding site, or the proximity of binding to probes of a detection reagent to their detection binding sites, allows differentiation of mutant and reference sequences for a mutant described herein (e.g., a rearrangement having a breakpoint described herein, e.g., in FIG. 1A or 1B or in the section herein entitled Nucleic Acid Molecules, from a reference sequence. The detection reagent can be modified, e.g., with a label or other moiety, e.g., a moiety that allows capture.

In embodiments, a mutation described herein, e.g., in FIG. 1A or 1B or in the section herein entitled Nucleic Acid Molecules, is distinguished from reference by binding or lack of binding of a detection reagent.

In embodiments, e.g., with proximity based probes, e.g., FISH probes, a mutation described herein, e.g., in FIG. 1A or 1B or in the section herein entitled Nucleic Acid Molecules, and a reference are distinguished by the proximity of the binding of two probes of the detection reagent. E.g., a genomic rearrangement that alters the distance between two binding sites can be detected with proximity based probes, e.g., FISH probes.

In an embodiment, the detection reagent comprises a nucleic acid molecule, e.g., a DNA, RNA or mixed DNA/RNA molecule, which, e.g., in its target binding site, includes the interrogation position, e.g., one or more of the nucleotides that flank a fusion junction, and which can distinguish (e.g., by affinity of binding of the detection reagent to a target nucleic acid, e.g., a genomic or transcribed nucleic acid, e.g., a cDNA or RNA, or the ability for a reaction, e.g., a ligation or extension reaction with the detection reagent) between a mutation, e.g., a translocation described herein, and a reference sequence. In embodiments, the interrogation position, e.g., one or both nucleotides flanking the fusion junction can correspond to a terminal, e.g., to a 3′ or 5′ terminal nucleotide, a nucleotide immediately adjacent to a 3′ or 5′ terminal nucleotide, or to another internal nucleotide, of the detection reagent or target binding site.

In embodiments, the difference in the affinity of the detection reagent for a target nucleic acid, e.g., a genomic or transcribed nucleic acid, e.g., a cDNA or RNA, comprising the mutant, e.g., a rearrangement or fusion junction, described in FIG. 1A or 1B or in the section herein entitled Nucleic Acid Molecules, and that for a target nucleic acid comprising the reference sequence allows determination of the presence or absence of the mutation (or reference) sequence. Typically, such detection reagents, under assay conditions, will exhibit substantially higher levels of binding only to the mutant or only to the reference sequence.

In embodiments, binding allows (or inhibits) a subsequent reaction, e.g., a subsequent reaction involving the detection reagent or the target nucleic acid. E.g., binding can allow ligation, or the addition of one or more nucleotides to a nucleic acid, e.g., the detection reagent, e.g., by DNA polymerase, which can be detected and used to distinguish mutant from reference. In embodiments, the interrogation position, e.g., one or both nucleotides flanking the fusion junction is located at the terminus, or sufficiently close to the terminus, of the detection reagent or its target binding site, such that hybridization, or a chemical reaction, e.g., the addition of one or more nucleotides to the detection reagent, e.g., by DNA polymerase, only occurs, or occurs at a substantially higher rate, when there is a perfect match between the detection reagent and the target nucleic acid at the interrogation position, e.g., one or both nucleotides flanking the fusion junction or at a nucleotide position within 1, 2, or 3 nucleotides of the interrogation position, e.g., one or both nucleotides flanking the fusion junction.

In an embodiment, the detection reagent comprises a nucleic acid, e.g., a DNA, RNA or mixed DNA/RNA molecule wherein the molecule, or its target binding site, is adjacent (or flanks), e.g., directly adjacent, to the interrogation position, e.g., one or more of the nucleotides that flank a fusion junction, and which can distinguish between a mutation, e.g., a mutant, e.g., a rearrangement or fusion junction, described in FIG. 1A or 1B or in the section herein entitled Nucleic Acid Molecules, and a reference sequence, in a target nucleic acid, e.g., a genomic or transcribed nucleic acid, e.g., a cDNA or RNA.

In embodiments, the detection reagent binding site is adjacent to the interrogation position, e.g., one or both nucleotides flanking the fusion junction, e.g., the 5′ or 3′ terminal nucleotide of the detection reagent, or its target binding site, is adjacent, e.g., between 0 (directly adjacent) and 1,000, 500, 400, 200, 100, 50, 10, 5, 4, 3, 2, or 1 nucleotides from the interrogation position, e.g., one or both nucleotides flanking the fusion junction. In embodiments, the outcome of a reaction will vary with the identity of the nucleotide at the interrogation position, e.g., one or both nucleotides flanking the fusion junction, allowing one to distinguish between mutant and reference sequences. E.g., in the presence of a first nucleotide at the interrogation position, e.g., one or both nucleotides flanking the fusion junction, a first reaction will be favored over a second reaction. E.g., in a ligation or primer extension reaction, the product will differ, e.g., in charge, sequence, size, or susceptibility to a further reaction (e.g., restriction cleavage) depending on the identity of the nucleotide at the interrogation position, e.g., one or both nucleotides flanking the fusion junction. In embodiments the detection reagent comprises a set of reagents, for example paired molecules (e.g., forward and reverse primers), allowing for amplification, e.g., by PCR amplification, of a duplex containing the interrogation position, e.g., one or both nucleotides flanking the fusion junction. In such embodiments, the presence of the mutation can be determined by a difference in the property of the amplification product, e.g., size, sequence, charge, or susceptibility to a reaction, resulting from a sequence comprising the interrogation position, e.g., one or both nucleotides flanking the fusion junction, and a corresponding sequence having a reference nucleotide at the interrogation position, e.g., one or both nucleotides flanking the fusion junctions. In embodiments, the presence or absence of a characteristic amplification product is indicative of the identity of the nucleotide at the interrogation site and thus allows detection of the mutation.

In embodiments, the detection reagent, or its target binding site, is directly adjacent to the interrogation position, e.g., one or both nucleotides flanking the fusion junction, e.g., the 5′ or 3′ terminal nucleotide of the detection reagent is directly adjacent to the interrogation position, e.g., one or both nucleotides flanking the fusion junction. In embodiments, the identity of the nucleotide at the interrogation position, e.g., one or both nucleotides flanking the fusion junction, will determine the nature of a reaction, e.g., a reaction involving the detection reagent, e.g., the modification of one end of the detection reagent. E.g., in the presence of a first nucleotide at the interrogation position, e.g., one or both nucleotides flanking the fusion junction, a first reaction will be favored over a second reaction. By way of example, the presence of a first nucleotide at the interrogation position, e.g., one or both nucleotides flanking the fusion junction, e.g., a nucleotide associated with a mutation, can promote a first reaction, e.g., the addition of a complementary nucleotide to the detection reagent. By way of example, the presence of an A at the interrogation position, e.g., one or both nucleotides flanking the fusion junction, will cause the incorporation of a T, having, e.g., a first colorimetric label, while the presence of a G and the interrogation position, e.g., one or both nucleotides flanking the fusion junction, will cause the incorporation for a C, having, e.g., a second colorimetric label. In an embodiment, the presence of a first nucleotide at the nucleotide will result in ligation of the detection reagent to a second nucleic acid. E.g., a third nucleic acid can be hybridized to the target nucleic acid sufficiently close to the interrogation site that if the third nucleic acid has an exact match at the interrogation site it will be ligated to the detection reagent. Detection of the ligation product, or its absence, is indicative of the identity of the nucleotide at the interrogation site and thus allows detection of the mutation.

A variety of readouts can be employed. E.g., binding of the detection reagent to the mutant or reference sequence can be followed by a moiety, e.g., a label, associated with the detection reagent, e.g., a radioactive or enzymatic label. In embodiments the label comprises a quenching agent and a signaling agent and hybridization results in altering the distance between those two elements, e.g., increasing the distance and un-quenching the signaling agent. In embodiments, the detection reagent can include a moiety that allows separation from other components of a reaction mixture. In embodiments, binding allows cleavage of the bound detection reagent, e.g., by an enzyme, e.g., by the nuclease activity of the DNA polymerase or by a restriction enzyme. The cleavage can be detected by the appearance or disappearance of a nucleic acid or by the separation of a quenching agent and a signaling agent associated with the detection reagent. In embodiments, binding protects, or renders the target susceptible, to further chemical reaction, e.g., labeling or degradation, e.g., by restriction enzymes. In embodiments binding with the detection reagent allows capture separation or physical manipulation of the target nucleic acid to thereby allow for identification. In embodiments binding can result in a detect localization of the detection reagent or target, e.g., binding could capture the target nucleic acid or displace a third nucleic acid. Binding can allow for determination of the presence of mutant or reference sequences with FISH, particularly in the case of rearrangements. Binding can allow for the extension or other size change in a component, e.g., the detection reagent, allowing distinction between mutant and reference sequences. Binding can allow for the production, e.g., by PCR, of an amplicon that distinguishes mutant from reference sequence.

In an embodiment the detection reagent, or the target binding site, is between 5 and 2000, 5 and 1000, 5 and 500, 5 and 300, 5 and 250, 5 and 200, 5 and 150, 5 and 100, 5 and 50, 5 and 25, 5 and 20, 5 and 15, or 5 and 10 nucleotides in length. In an embodiment the detection reagent, or the target binding site, is between 10 and 2000, 10 and 1000, 10 and 500, 10 and 300, 10 and 250, 10 and 200, 10 and 150, 10 and 100, 10 and 50, 10 and 25, 10 and 20, or 10 and 15, nucleotides in length. In an embodiment the detection reagent, or the target binding site, is between 10 and 2000, 10 and 1000, 20 and 500, 20 and 300, 20 and 250, 20 and 200, 20 and 150, 20 and 100, 20 and 50, or 20 and 25 nucleotides in length. In an embodiment the detection reagent, or the target binding site, is sufficiently long to distinguish between mutant and reference sequences and is less than 100, 200, 300, 400, 500, 1,000, 1,500, and 2,000 nucleotides in length.

In embodiments, the detection reagent comprises two probes which will bind with a first proximity to one another if a mutation described herein, e.g., a rearrangement or fusion junction, described in FIG. 1A or 1B or in the section herein entitled Nucleic Acid Molecules, is present and with a second proximity if the mutation is not present. Typically, one of the proximities will result in production of a signal and the other will not. E.g., one probe can comprise a signal generator and the other can comprise a signal quencher. If the proximity is close there will be no signal and if the proximity is less close then signal will be produced.

### Preparations of Mutant Nucleic Acid and Uses Thereof

In another aspect, the invention features purified or isolated preparations of a neoplastic or tumor cell nucleic acid, e.g., DNA, e.g., genomic DNA or cDNA, or RNA, containing an interrogation position described herein, useful for determining if a mutation disclosed herein is present. The nucleic acid includes the interrogation position, and typically additional fusion sequence on one or both sides of the interrogation position. In addition the nucleic acid can contain heterologous sequences, e.g., adaptor or priming sequences, typically attached to one or both terminus of the nucleic acid. The nucleic acid also includes a label or other moiety. e.g., a moiety that allows separation or localization.

In embodiments, the nucleic acid is between 20 and 1,000, 30 and 900, 40 and 800, 50 and 700, 60 and 600, 70 and 500, 80 and 400, 90 and 300, or 100 and 200 nucleotides in length (with or without heterologous sequences). In one embodiment, the nucleic acid is between 40 and 1,000, 50 and 900, 60 and 800, 70 and 700, 80 and 600, 90 and 500, 100 and 400, 110 and 300, or 120 and 200 nucleotides in length (with or without heterologous sequences). In another embodiment, the nucleic acid is between 50 and 1.000, 50 and 900, 50 and 800, 50 and 700, 50 and 600, 50 and 500, 50 and 400, 50 and 300, or 50 and 200 nucleotides in length (with or without heterologous sequences). In embodiments, the nucleic acid is of sufficient length to allow sequencing (e.g., by chemical sequencing or by determining a difference in Tₘ between mutant and reference preparations) but is optionally less than 100, 200, 300, 400, or 500 nucleotides in length (with or without heterologous sequences).

Such preparations can be used to sequence nucleic acid from a sample, e.g., a neoplastic or tumor sample. In an embodiment the purified preparation is provided by in situ amplification of a nucleic acid provided on a substrate. In embodiments the purified preparation is spatially distinct from other nucleic acids, e.g., other amplified nucleic acids, on a substrate.

In an embodiment, the purified or isolated preparation of nucleic acid is derived from a neoplasm or tumor of a type described herein, e.g., neoplasm and/or cancer, e.g., a melanocytic neoplasm, melanoma or metastatic cancer. In one embodiment, the fusion nucleic acid is derived from a histiocytosis, e.g., a non-Langerhans cell histiocytosis.

Such preparations can be used to determine if a sample comprises mutant sequence, e.g., a translocation as described herein. In one embodiment, the translocation includes a breakpoint. Nucleic acids that include the aforesaid breakpoint, e.g., a breakpoint described herein, are collectively referred to herein as fusion nucleic acids.

In another aspect, the invention features, a method of determining the sequence of an interrogation position for a mutation described herein, comprising:

providing a purified or isolated preparations of nucleic acid or fusion nucleic acid, e.g., DNA, e.g., genomic DNA or cDNA, or RNA, containing an interrogation position described herein,

sequencing, by a method that breaks or forms a chemical bond, e.g., a covalent or non-covalent chemical bond, e.g., in a detection reagent or a target sequence, the nucleic acid so as to determine the identity of the nucleotide at an interrogation position. The method allows determining if a mutation described herein is present.

In an embodiment, sequencing comprises contacting the fusion nucleic acid with a detection reagent described herein.

In an embodiment, sequencing comprises determining a physical property, e.g., stability of a duplex form of the fusion nucleic acid (e.g., Tₘ) that can distinguish mutant from reference sequence.

In an embodiment, the fusion nucleic acid is derived from a neoplasm or a tumor of a type described herein, e.g., a melanocytic neoplasm, melanoma or metastatic cancer. In one embodiment, the fusion nucleic acid is derived from a histiocytosis, e.g., a non-Langerhans cell histiocytosis.

### Reaction Mixtures and Devices

In another aspect, the invention features, a reaction mixture comprising:

a) a sample, or nucleic acid. e.g., DNA, e.g., genomic DNA or cDNA, or RNA, e.g., from a cancer, containing:

an interrogation position for a mutation, e.g., a rearrangement or fusion junction, described in FIG. 1A, 1B or 1C or in the section herein entitled Nucleic Acid Molecules; or

a mutation, e.g., a rearrangement or fusion junction, described in FIG. 1A or 1B or in the section herein entitled Nucleic Acid Molecules; and

b) a detection reagent described herein, e.g., a detection reagent described in the section herein entitled, Detection Reagents and Detection of Mutations, e.g., in the section herein entitled, Nucleic Acid-based Detection Reagents.

In an embodiment, the sample comprises nucleic acid from a cancer.

In an embodiment the sample, or nucleic acid in the sample, is from a cancer listed in FIG. 1A, and the detection reagent detects a mutant, e.g., a rearrangement or fusion junction disclosed in FIG. 1A, 1B or 1C or in the section herein entitled Nucleic Acid Molecules.

In an embodiment, the sample, or nucleic acid in the sample, is from a cancer listed in FIG. 1A, and the detection reagent detects a mutant, e.g., a rearrangement or fusion junction disclosed in FIG. 1A, 1B or 1C or in the section herein entitled Nucleic Acid Molecules, in a fusion of the two genes in the fusion associated with that cancer in FIG. 1A.

In an embodiment:

the sample, or nucleic acid in the sample, is from a cancer, e.g., a cancer as described herein, and the detection reagent is one that detects a fusion of the CLIP1 and ROS1 genes, e.g., a detection reagent that detects a mutant, e.g., a rearrangement or fusion junction described in FIG. 1A, 1B or 1C or in the section herein entitled Nucleic Acid Molecules, for a fusion of CLIP1 and ROS1;

the sample, or nucleic acid in the sample, is from a cancer, e.g., a cancer as described herein, and the detection reagent is one that detects a fusion of the PPFIBP1 and ROS1 genes, e.g., a detection reagent that detects a mutant, e.g., a rearrangement or fusion junction described in FIG. 1A, 1B or 1C or in the section herein entitled Nucleic Acid Molecules, for a fusion of PPFIBP1 and ROS1;

the sample, or nucleic acid in the sample, is from a cancer, e.g., a cancer as described herein, and the detection reagent is one that detects a fusion of the TPM3 and ROS1 genes, e.g., a detection reagent that detects a mutant, e.g., a rearrangement or fusion junction described in FIG. 1A, 1B or 1C or in the section herein entitled Nucleic Acid Molecules, for a fusion of TPM3 and ROS1;

the sample, or nucleic acid in the sample, is from a cancer, e.g., a cancer as described herein, and the detection reagent is one that detects a fusion of the ZCCHC8 and ROS1 genes, e.g., a detection reagent that detects a mutant, e.g., a rearrangement or fusion junction described in FIG. 1A, 1B or 1C or in the section herein entitled Nucleic Acid Molecules, for a fusion of ZCCHC8 and ROS1;

the sample, or nucleic acid in the sample, is from a cancer. e.g., a cancer as described herein, and the detection reagent is one that detects a fusion of the MYO5A and ROS1 genes, e.g., a detection reagent that detects a mutant, e.g., a rearrangement or fusion junction described in FIG. 1A, 1B or 1C or in the section herein entitled Nucleic Acid Molecules, for a fusion of MYO5A and ROS1;

the sample, or nucleic acid in the sample, is from a cancer, e.g., a cancer as described herein, and the detection reagent is one that detects a fusion of the PWWP2A and ROS1 genes, e.g., a detection reagent that detects a mutant, e.g., a rearrangement or fusion junction described in FIG. 1A, 1B or 1C or in the section herein entitled Nucleic Acid Molecules, for a fusion of PWWP2A and ROS1;

the sample, or nucleic acid in the sample, is from a cancer, e.g., a cancer as described herein, and the detection reagent is one that detects a fusion of the HLA-A and ROS1 genes. e.g., a detection reagent that detects a mutant, e.g., a rearrangement or fusion junction described in FIG. 1A, 1B or 1C or in the section herein entitled Nucleic Acid Molecules, for a fusion of HLA-A and ROS1;

the sample, or nucleic acid in the sample, is from a cancer, e.g., a cancer as described herein, and the detection reagent is one that detects a fusion of the ERC1 and ROS1 genes, e.g., a detection reagent that detects a mutant, e.g., a rearrangement or fusion junction described in FIG. 1A, 1B or 1C or in the section herein entitled Nucleic Acid Molecules, for a fusion of ERC1 and ROS1;

the sample, or nucleic acid in the sample, is from a cancer, e.g., a cancer as described herein, and the detection reagent is one that detects a fusion of the TPM3 and ALK genes, e.g., a detection reagent that detects a mutant, e.g., a rearrangement or fusion junction described in FIG. 1A, 1B or 1C or in the section herein entitled Nucleic Acid Molecules, for a fusion of TPM3 and ALK:

the sample, or nucleic acid in the sample, is from a cancer, e.g., a cancer as described herein, and the detection reagent is one that detects a fusion of the GOLGA5 and RET genes, e.g., a detection reagent that detects a mutant, e.g., a rearrangement or fusion junction described in FIG. 1A, 1B or 1C or in the section herein entitled Nucleic Acid Molecules, for a fusion of GOLGA5 and RET;

the sample, or nucleic acid in the sample, is from a cancer, e.g., a cancer as described herein, and the detection reagent is one that detects a fusion of the CEP89 and BRAF genes, e.g., a detection reagent that detects a mutant, e.g., a rearrangement or fusion junction described in FIG. 1A, 1B or 1C or in the section herein entitled Nucleic Acid Molecules, for a fusion of CEP89 and BRAF;

the sample, or nucleic acid in the sample, is from a cancer, e.g., a cancer as described herein, and the detection reagent is one that detects a fusion of the KIF5B and RET genes, e.g., a detection reagent that detects a mutant, e.g., a rearrangement or fusion junction described in FIG. 1A, 1B or 1C or in the section herein entitled Nucleic Acid Molecules, for a fusion of KIF5B and RET;

the sample, or nucleic acid in the sample, is from a cancer, e.g., a cancer as described herein, and the detection reagent is one that detects a fusion of the TP53 and NTRK1 genes, e.g., a detection reagent that detects a mutant, e.g., a rearrangement or fusion junction described in FIG. 1A, 1B or 1C or in the section herein entitled Nucleic Acid Molecules, for a fusion of TP53 and NTRK1;

the sample, or nucleic acid in the sample, is from a cancer. e.g., a cancer as described herein, and the detection reagent is one that detects a fusion of the KIAA1598 and ROS1 genes, e.g., a detection reagent that detects a mutant, e.g., a rearrangement or fusion junction described in FIG. 1A, 1B or 1C or in the section herein entitled Nucleic Acid Molecules, for a fusion of KIAA1598 and ROS1;

the sample, or nucleic acid in the sample, is from a cancer, e.g., a cancer as described herein, and the detection reagent is one that detects a fusion of the DCTN1 and ALK genes, e.g., a detection reagent that detects a mutant, e.g., a rearrangement or fusion junction described in FIG. 1A, 1B or 1C or in the section herein entitled Nucleic Acid Molecules, for a fusion of DCTN1 and ALK:

the sample, or nucleic acid in the sample, is from a cancer, e.g., a cancer as described herein, and the detection reagent is one that detects a fusion of the LSM14A and BRAF genes, e.g., a detection reagent that detects a mutant, e.g., a rearrangement or fusion junction described in FIG. 1A, 1B or 1C or in the section herein entitled Nucleic Acid Molecules, for a fusion of LSM14A and BRAF:

the sample, or nucleic acid in the sample, is from a cancer, e.g., a cancer as described herein, and the detection reagent is one that detects a fusion of the LMNA and NTRK1 genes, e.g., a detection reagent that detects a mutant, e.g., a rearrangement or fusion junction described in FIG. 1A, 1B or 1C or in the section herein entitled Nucleic Acid Molecules, for a fusion of LMNA and NTRK1. In another aspect, the invention features, purified or isolated preparations of a fusion nucleic acid, e.g., DNA, e.g., genomic DNA or cDNA, or RNA, containing an interrogation position, e.g., one or both nucleotides flanking the fusion junction, described herein or a mutation, e.g., a rearrangement or fusion junction, described in FIG. 1A, 1B or 1C or in the section herein entitled Nucleic Acid Molecules. In embodiments the preparation is useful for determining if a mutation disclosed herein is present. In embodiments the preparation is disposed in a device, e.g., a sequencing device, or a sample holder for use in such a device. In an embodiment, the fusion nucleic acid is derived from a neoplasm or a tumor of a type described herein, for example melanoma, particularly melanoma with spitzoid histopathological features. In an embodiment the nucleic acid is from a translocation listed in FIG. 1A. In an embodiment the nucleic acid is from a translocation listed in FIG. 1A and the device also includes a detection reagent such as one that detects a fusion of the genes associate with that cancer and listed in FIG. 1A, e.g., a detection reagent that detects a mutant, e.g., a rearrangement or fusion junction described in FIG. 1A, 1B or 1C or in the section herein entitled Nucleic Acid Molecules, for a fusion of the genes that are the fusion partners forming the fusion associated with the cancer and listed in FIG. 1A.

In another aspect, the invention features, purified or isolated preparations of a fusion nucleic acid, e.g., DNA, e.g., genomic DNA or cDNA, or RNA, containing an interrogation position, e.g., one or both nucleotides flanking the fusion junction, described herein or a mutation, e.g., a rearrangement or fusion junction, described in FIG. 1A or 1B or in the section herein entitled Nucleic Acid Molecules, useful for determining if a mutation disclosed herein is present, disposed in a device for determining a physical or chemical property, e.g., stability of a duplex, e.g., Tₘ or a sample holder for use in such a device. In an embodiment, the device is a calorimeter. In an embodiment the fusion nucleic acid is derived from a neoplasm or a tumor of a type described herein, e.g., in FIG. 1A.

The detection reagents described herein can be used to determine if a mutation described herein is present in a sample. In embodiments, the sample comprises a nucleic acid that is derived from a neoplastic or a tumor cell, e.g. a cancer described in FIG. 1A. The cell can be from a neoplastic or a tumor sample, e.g., a biopsy taken from the neoplasm or the tumor; from circulating tumor cells, e.g., from peripheral blood; or from a blood or plasma sample.

In another aspect, the invention features, a method of making a reaction mixture by combining:

a) a sample, or nucleic acid. e.g., DNA, e.g., genomic DNA or cDNA, or RNA, e.g., from a cancer, containing:

an interrogation position for a mutation, e.g., a rearrangement or fusion junction, described in FIG. 1A, 1B or 1C or in the section herein entitled Nucleic Acid Molecules; or

a mutation, e.g., a rearrangement or fusion junction, described in FIG. 1A, 1B or 1C or in the section herein entitled Nucleic Acid Molecules; and

b) a detection reagent described herein, e.g., a detection reagent described in the section herein entitled, Detection Reagents and Detection of Mutations, e.g., in the section herein entitled, Nucleic Acid-based Detection Reagents.

A mutation described herein, can be distinguished from a reference, e.g., a non-mutant or wild type sequence, by reaction with an enzyme that reacts differentially with the mutation and the reference. E.g., they can be distinguished by cleavage with a restriction enzyme that has differing activity for the mutant and reference sequences. E.g., the invention includes a method of contacting a nucleic acid comprising a mutation described herein with such an enzyme and determining if a product of that cleavage which can distinguish mutant form reference sequence is present.

In one aspect the inventions provides, a purified preparation of a restriction enzyme cleavage product which can distinguish between mutant and reference sequence, wherein one end of the cleavage product is defined by an enzyme that cleaves differentially between mutant and reference sequence. In an embodiment, the cleavage product includes the interrogation position, e.g., one or both nucleotides flanking the fusion junction.

Protein-Based Detection Reagents, Methods. Reaction Mixtures and Devices

A mutant protein described herein can be distinguished from a reference, e.g., a non-mutant or wild-type protein, by reaction with a reagent, e.g., a substrate, e.g., a substrate for catalytic activity, e.g., phosphorylation or other fusion protein activity, or an antibody that reacts differentially with the mutant and reference protein. In one aspect, the invention includes a method of contacting a sample comprising a mutant protein described herein with such reagent and determining if the mutant protein is present in the sample.

Accordingly, in another aspect, the invention features, a reaction mixture comprising:

a) a sample, e.g., a cancer sample, comprising a fusion protein having fusion partners described in FIG. 1A, 1B or 1C, e.g., a fusion protein encoded by a mutation described in FIG. 1A, 1B or 1C or in the section herein entitled Nucleic Acid Molecules; and

b) a detection reagent, e.g., a substrate, e.g., a substrate for catalytic activity, e.g., phosphorylation or other fusion protein activity, or an antibody that reacts differentially with the mutant and reference protein.

In another aspect, the invention features, a method of making a reaction mixture comprising combining:

a) a sample, e.g., a cancer sample, comprising a fusion protein having fusion partners described in FIG. 1A, 1B or 1C, e.g., a fusion protein encoded by a mutation described in FIG. 1A, 1B or 1C or in the section herein entitled Nucleic Acid Molecules; and

b) a detection reagent, e.g., a substrate, e.g., a substrate for catalytic activity, e.g., phosphorylation or other fusion protein activity, or an antibody that reacts differentially with the mutant and reference protein.

### Kits

In another aspect, the invention features a kit comprising one or more detection reagents or sets of detection reagents as described herein.

### Methods Reducing a Fusion Molecule Activity

In another aspect, the invention features a method of reducing an activity of a fusion molecule described herein. The method includes contacting the fusion molecule, or a fusion molecule-expressing cell, with an agent that inhibits an activity or expression of the fusion molecule (e.g., an inhibitor, e.g., a kinase inhibitor). In one embodiment, the contacting step can be effected in vitro, e.g., in a cell lysate or in a reconstituted system. Alternatively, the method can be performed on cells in culture, e.g., in vitro or ex vivo. In other embodiments, the method can be performed on fusion molecule-expressing cells present in a subject, e.g., as part of an in vivo (e.g., therapeutic or prophylactic) protocol. In an embodiment the method is practiced on an animal subject (e.g., an in vivo animal model). In certain embodiments, the fusion molecule is a nucleic acid molecule or a polypeptide as described herein.

In a related aspect, a method of inhibiting, reducing, or treating a hyperproliferative disorder, e.g., a neoplasm (including benign, pre-malignant or malignant (e.g., a cancer), in a subject is provided. The method includes administering to the subject a preselected therapeutic agent. e.g., an anti-cancer agent (e.g., a kinase inhibitor), as a single agent, or in combination, in an amount sufficient to reduce, inhibit or treat the activity or expression of a fusion molecule described herein), thereby inhibiting, reducing, or treating the hyperproliferative disorder in the subject. “Treatment” as used herein includes, but is not limited to, inhibiting tumor growth, reducing tumor mass, reducing size or number of metastatic lesions, inhibiting the development of new metastatic lesions, prolonging survival, prolonging progression-free survival, prolonging time to progression, and/or enhancing quality of life.

In one embodiment, a kinase inhibitor is administered based on a determination that a fusion molecule described herein is present in a subject, e.g., based on its presence in a subject's sample. Thus, treatment can be combined with fusion molecule detection or evaluation method, e.g., as described herein, or administered in response to a determination made by a fusion molecule detection or evaluation method, e.g., as described herein. In certain embodiments, the kinase inhibitor is administered responsive to acquiring knowledge or information of the presence of the fusion molecule in a subject. In one embodiment, the kinase inhibitor is administered responsive to acquiring knowledge or information on the subject's genotype, e.g., acquiring knowledge or information that the patient's genotype has a fusion molecule. In other embodiments, the kinase inhibitor is administered responsive to receiving a communication (e.g., a report) of the presence of the fusion molecule in a subject (e.g., a subject's sample). In yet other embodiments, the kinase inhibitor is administered responsive to information obtained from a collaboration with another party that identifies the presence of the fusion molecule in a subject (e.g., a subject's sample). In other embodiments, the kinase inhibitor is administered responsive to a determination that the fusion molecule is present in a subject. In one embodiment, the determination of the presence of the fusion molecule is carried out using one or more of the methods, e.g., the sequencing methods, described herein. In other embodiments, the determination of the presence of the fusion molecule includes receiving information on the subject's fusion molecule genotype, e.g., from another party or source.

The methods can, optionally, further include the step(s) of identifying (e.g., evaluating, diagnosing, screening, and/or selecting) a subject at risk of having, or having, a fusion molecule described herein. In one embodiment, the method further includes one or more of: acquiring knowledge or information of the presence of the fusion molecule in a subject (e.g., a subject's sample); acquiring knowledge or information on the subject's genotype, e.g., acquiring knowledge or information that the patient's genotype has a fusion molecule; receiving a communication (e.g., a report) of the presence of the fusion molecule in a subject (e.g., a subject's sample); or collaborating with another party that identifies the presence of the fusion molecule in a subject.

In one embodiment, the subject treated has a fusion molecule described herein; e.g., the subject has a tumor or cancer harboring a fusion molecule described herein. In other embodiments, the subject has been previously identified as having a fusion molecule described herein. In yet other embodiments, the subject has been previously identified as being likely or unlikely to respond to treatment with a protein kinase inhibitor, e.g., a subject that has previously participated in a clinical trial. In other embodiments, the subject has been previously identified as being likely or unlikely to respond to treatment with a protein kinase inhibitor, based on the presence of the fusion molecule described herein. In one embodiment, the subject is a mammal, e.g., a human. In one embodiment, the subject has, or is at risk of having a cancer at any stage of disease. In other embodiments, the subject is a patient, e.g., a cancer patient.

In other embodiments, the subject treated is a cancer patient who has participated in a clinical trial. For example, the subject participated in a clinical trial that evaluated a kinase inhibitor (e.g., a multikinase inhibitor, a specific kinase inhibitor). In other embodiment, the subject participated in a clinical trial that evaluates upstream or downstream targets of the specific kinase. In one embodiment, said cancer patient responded to the kinase inhibitor evaluated.

In certain embodiments, the neoplasm or neoplastic cell is a benign, pre-malignant, malignant (cancer) or metastasis. In certain embodiments, the cancer is a solid tumor, a soft tissue tumor, or a metastatic lesion. In one embodiment, the cancer is chosen from lung adenocarcinoma, cervical adenocarcinoma, uterus endometrial adenocarcinoma, glioblastoma, melanoma, spindle cell sarcoma, ameloblastic fibrosarcoma, adenocarcinoma, cholangiocarcinoma, urothelial (transitional cell) carcinoma, ovarian epithelial carcinoma, colorectal adenocarcinoma, breast carcinoma, prostate carcinoma, or pancreas ductal adenocarcinoma. In one embodiment, the cancer is chosen from a lung cancer, a pancreatic cancer, melanoma, a colorectal cancer, an esophageal-gastric cancer, a thyroid cancer, or an adenocarcinoma. In other embodiment, the lung cancer is chosen from one or more of the following: non-small cell lung cancer (NSCLC), small cell lung cancer (SCLC), squamous cell carcinoma (SCC), adenocarcinoma of the lung, bronchogenic carcinoma, or a combination thereof. In one embodiment, the lung cancer is NSCLC or SCC.

In one embodiment, the anti-cancer agent is a kinase inhibitor. For example, the kinase inhibitor is a multi-kinase inhibitor or a specific inhibitor.

In other embodiments, the anti-cancer agent is an antagonist of a fusion molecule described herein which inhibits the expression of nucleic acid encoding the fusion molecule. Examples of such fusion molecule antagonists include nucleic acid molecules, for example, antisense molecules, ribozymes, RNAi, triple helix molecules that hybridize to a nucleic acid encoding a fusion molecule described herein, or a transcription regulatory region, and blocks or reduces mRNA expression of the fusion molecule.

In other embodiments, the inhibitor, e.g., kinase inhibitor, is administered in combination with a second therapeutic agent or a different therapeutic modality, e.g., anti-cancer agents, and/or in combination with surgical and/or radiation procedures. For example, the second therapeutic agent can be a cytotoxic or a cytostatic agent. Exemplary cytotoxic agents include antimicrotubule agents, topoisomerase inhibitors, or taxanes, antimetabolites, mitotic inhibitors, alkylating agents, intercalating agents, agents capable of interfering with a signal transduction pathway, agents that promote apoptosis and radiation. In yet other embodiments, the methods can be used in combination with immunomodulatory agents, e.g., IL-1, 2, 4, 6, or 12, or interferon alpha or gamma, or immune cell growth factors such as GM-CSF.

### Screening Methods

In another aspect, the invention features a method, or assay, for screening for agents that modulate, e.g., inhibit, the expression or activity of a fusion molecule described herein. The method includes contacting a fusion molecule described herein, or a cell expressing a fusion molecule described herein, with a candidate agent; and detecting a change in a parameter associated with a fusion molecule described herein, e.g., a change in the expression or an activity of the fusion molecule. The method can, optionally, include comparing the treated parameter to a reference value, e.g., a control sample (e.g., comparing a parameter obtained from a sample with the candidate agent to a parameter obtained from a sample without the candidate agent). In one embodiment, if a decrease in expression or activity of the fusion molecule is detected, the candidate agent is identified as an inhibitor. In another embodiment, if an increase in expression or activity of the fusion molecule is detected, the candidate agent is identified as an activator. In certain embodiments, the fusion molecule is a nucleic acid molecule or a polypeptide as described herein.

In one embodiment, the contacting step is carried out in a cell-free system. e.g., a cell lysate or in a reconstituted system. In other embodiments, the contacting step is effected in a cell in culture, e.g., a cell expressing a fusion molecule described herein (e.g., a mammalian cell, a tumor cell or cell line, a recombinant cell). In yet other embodiments, the contacting step is effected in a cell in vivo (a fusion molecule-expressing cell present in a subject, e.g., an animal subject (e.g., an in vivo animal model).

Exemplary parameters evaluated include one or more of:

(i) a change in binding activity, e.g., direct binding of the candidate agent to a fusion polypeptide described herein; a binding competition between a known ligand and the candidate agent to a fusion polypeptide described herein;

(ii) a change in kinase activity, e.g., phosphorylation levels of a fusion polypeptide described herein (e.g., an increased or decreased autophosphorylation); or a change in phosphorylation of a target of an kinase. In certain embodiments, a change in kinase activity, e.g., phosphorylation, is detected by any of Western blot (e.g., using an antibody specific for either of the genes associated with a fusion molecule described herein; a phosphor-specific antibody, detecting a shift in the molecular weight of a fusion polypeptide described herein), mass spectrometry, immunoprecipitation, immunohistochemistry, immunomagnetic beads, among others;

(iii) a change in an activity of a cell containing a fusion molecule described herein (e.g., a tumor cell or a recombinant cell), e.g., a change in proliferation, morphology or tumorigenicity of the cell;

(iv) a change in tumor present in an animal subject, e.g., size, appearance, proliferation, of the tumor; or

(v) a change in the level, e.g., expression level, of a fusion polypeptide or nucleic acid molecule described herein.

In one embodiment, a change in a cell free assay in the presence of a candidate agent is evaluated. For example, an activity of a fusion molecule described herein, or interaction of a fusion molecule described herein with a downstream ligand can be detected. In one embodiment, a fusion polypeptide described herein is contacted with a ligand, e.g., in solution, and a candidate agent is monitored for an ability to modulate, e.g., inhibit, an interaction, e.g., binding, between the fusion polypeptide and the ligand.

In other embodiments, a change in an activity of a cell is detected in a cell in culture, e.g., a cell expressing a fusion molecule described herein (e.g., a mammalian cell, a tumor cell or cell line, a recombinant cell). In one embodiment, the cell is a recombinant cell that is modified to express a fusion nucleic acid described herein, e.g., is a recombinant cell transfected with a fusion nucleic acid described herein. The transfected cell can show a change in response to the expressed fusion molecule, e.g., increased proliferation, changes in morphology, increased tumorigenicity, and/or acquired a transformed phenotype. A change in any of the activities of the cell, e.g., the recombinant cell, in the presence of the candidate agent can be detected. For example, a decrease in one or more of: proliferation, tumorigenicity, transformed morphology, in the presence of the candidate agent can be indicative of an inhibitor of a fusion molecule described herein. In other embodiments, a change in binding activity or phosphorylation as described herein is detected.

In yet other embodiment, a change in a tumor present in an animal subject (e.g., an in vivo animal model) is detected. In one embodiment, the animal model is a tumor containing animal or a xenograft comprising cells expressing a fusion molecule described herein (e.g., tumorigenic cells expressing a fusion molecule described herein). The candidate agent can be administered to the animal subject and a change in the tumor is detected. In one embodiment, the change in the tumor includes one or more of a tumor growth, tumor size, tumor burden, survival, is evaluated. A decrease in one or more of tumor growth, tumor size, tumor burden, or an increased survival is indicative that the candidate agent is an inhibitor.

In other embodiments, a change in expression of a fusion molecule described herein can be monitored by detecting the nucleic acid or protein levels, e.g., using the methods described herein.

In certain embodiments, the screening methods described herein can be repeated and/or combined. In one embodiment, a candidate agent that is evaluated in a cell-free or cell-based described herein can be further tested in an animal subject.

In one embodiment, the candidate agent is a small molecule compound, e.g., a kinase inhibitor, a nucleic acid (e.g., antisense, siRNA, aptamer, ribozymes, microRNA), an antibody molecule (e.g., a full antibody or antigen binding fragment thereof that binds to a gene of a fusion molecule described herein). The candidate agent can be obtained from a library (e.g., a commercial library of kinase inhibitors) or rationally designed (e.g., based on the kinase domain of a fusion described herein).

### Methods for Detecting Fusions

In another aspect, the invention features a method of determining the presence of a fusion as described herein. In one embodiment, the fusion is detected in a nucleic acid molecule or a polypeptide. The method includes detecting whether a fusion nucleic acid molecule or polypeptide is present in a cell (e.g., a circulating cell), a tissue (e.g., a tumor), or a sample, e.g., a tumor sample, from a subject. In one embodiment, the sample is a nucleic acid sample. In one embodiment, the nucleic acid sample comprises DNA, e.g., genomic DNA or cDNA, or RNA, e.g., mRNA. In other embodiments, the sample is a protein sample.

In one embodiment, the sample is, or has been, classified as non-malignant using other diagnostic techniques, e.g., immunohistochemistry.

In one embodiment, the sample is acquired from a subject (e.g., a subject having or at risk of having a cancer, e.g., a patient), or alternatively, the method further includes acquiring a sample from the subject. The sample can be chosen from one or more of: tissue, e.g., cancerous tissue (e.g., a tissue biopsy), whole blood, serum, plasma, buccal scrape, sputum, saliva, cerebrospinal fluid, urine, stool, circulating tumor cells, circulating nucleic acids, or bone marrow. In certain embodiments, the sample is a tissue (e.g., a tumor biopsy), a circulating tumor cell or nucleic acid.

In one embodiment, the cancer is chosen from lung adenocarcinoma, cervical adenocarcinoma, uterus endometrial adenocarcinoma, glioblastoma, melanoma, spindle cell sarcoma, ameloblastic fibrosarcoma, adenocarcinoma, cholangiocarcinoma, urothelial (transitional cell) carcinoma, ovarian epithelial carcinoma, colorectal adenocarcinoma, breast carcinoma, prostate carcinoma, or pancreas ductal adenocarcinoma. In embodiments, the tumor is from a cancer described herein, e.g., is chosen from a lung cancer, a colorectal cancer, an esophageal-gastric cancer, a thyroid cancer, an adenocarcinoma or a melanoma. In one embodiment, the tumor is from a lung cancer, e.g., a NSCLC, a SCLC, a SCC, or a combination thereof.

In one embodiment, the subject is at risk of having, or has a cancer (e.g., a patient with a cancer described herein).

In other embodiments, the fusion molecule is detected in a nucleic acid molecule by a method chosen from one or more of: nucleic acid hybridization assay, amplification-based assays (e.g., polymerase chain reaction (PCR)), PCR-RFLP assay, real-time PCR, sequencing, screening analysis (including metaphase cytogenetic analysis by standard karyotype methods, FISH (e.g., break away FISH), spectral karyotyping or MFISH, comparative genomic hybridization), in situ hybridization, SSP, HPLC or mass-spectrometric genotyping.

In one embodiment, the method includes: contacting a nucleic acid sample, e.g., a genomic DNA sample (e.g., a chromosomal sample or a fractionated, enriched or otherwise pre-treated sample) or a gene product (mRNA, cDNA), obtained from the subject, with a nucleic acid fragment (e.g., a probe or primer as described herein (e.g., an exon-specific probe or primer) under conditions suitable for hybridization, and determining the presence or absence of the fusion nucleic acid molecule. The method can, optionally, include enriching a sample for the gene or gene product.

In a related aspect, a method for determining the presence of a fusion nucleic acid molecule described herein is provided. The method includes: acquiring a sequence for a position in a nucleic acid molecule, e.g., by sequencing at least one nucleotide of the nucleic acid molecule (e.g., sequencing at least one nucleotide in the nucleic acid molecule that comprises the fusion), thereby determining that the fusion molecule is present in the nucleic acid molecule. Optionally, the sequence acquired is compared to a reference sequence, or a wild type reference sequence. In one embodiment, the nucleic acid molecule is from a cell (e.g., a circulating cell), a tissue (e.g., a tumor), or any sample from a subject (e.g., blood or plasma sample). In other embodiments, the nucleic acid molecule from a tumor sample (e.g., a tumor or cancer sample) is sequenced. In one embodiment, the sequence is determined by a next generation sequencing method. The method further can further include acquiring, e.g., directly or indirectly acquiring, a sample, e.g., a tumor or cancer sample, from a subject (e.g., a patient). In certain embodiments, the cancer is chosen from a lung cancer, colorectal cancer, esophageal-gastric cancer or melanoma.

In another aspect, the invention features a method of analyzing a tumor or a circulating tumor cell. The method includes acquiring a nucleic acid sample from the tumor or the circulating cell; and sequencing, e.g., by a next generation sequencing method, a nucleic acid molecule, e.g., a nucleic acid molecule that includes a fusion molecule as described herein.

In yet other embodiment, a fusion polypeptide is detected. The method includes: contacting a protein sample with a reagent which specifically binds to a fusion polypeptide described herein; and detecting the formation of a complex of the fusion polypeptide and the reagent. In one embodiment, the reagent is labeled with a detectable group to facilitate detection of the bound and unbound reagent. In one embodiment, the reagent is an antibody molecule, e.g., is selected from the group consisting of an antibody, and antibody derivative, and an antibody fragment.

In yet another embodiment, the level (e.g., expression level) or activity the fusion molecule is evaluated. For example, the level (e.g., expression level) or activity of the fusion molecule (e.g., mRNA or polypeptide) is detected and (optionally) compared to a pre-determined value, e.g., a reference value (e.g., a control sample).

In yet another embodiment, the fusion molecule is detected prior to initiating, during, or after, a treatment, e.g., treatment with a kinase inhibitor, in a subject having a fusion described herein.

In one embodiment, the fusion molecule is detected at the time of diagnosis with a cancer. In other embodiment, the fusion molecule is detected at a pre-determined interval, e.g., a first point in time and at least at a subsequent point in time.

In certain embodiments, responsive to a determination of the presence of the fusion molecule, the method further includes one or more of:

(1) stratifying a patient population (e.g., assigning a subject, e.g., a patient, to a group or class);

(2) identifying or selecting the subject as likely or unlikely to respond to a treatment, e.g., a kinase inhibitor treatment as described herein;

(3) selecting a treatment option, e.g., administering or not administering a preselected therapeutic agent, e.g., a kinase inhibitor as described herein; or

(4) prognosticating the time course of the disease in the subject (e.g., evaluating the likelihood of increased or decreased patient survival).

In certain embodiments, the kinase inhibitor is a multi-kinase inhibitor or a specific inhibitor.

In certain embodiments, responsive to the determination of the presence of a fusion molecule described herein, the subject is classified as a candidate to receive treatment with a kinase inhibitor, e.g., a kinase inhibitor as described herein. In one embodiment, responsive to the determination of the presence of a fusion molecule described herein, the subject, e.g., a patient, can further be assigned to a particular class if a fusion is identified in a sample of the patient. For example, a patient identified as having a fusion molecule described herein can be classified as a candidate to receive treatment with a kinase inhibitor, e.g., a specific kinase inhibitor as described herein. In one embodiment, the subject, e.g., a patient, is assigned to a second class if the mutation is not present. For example, a patient who has a lung tumor that does not contain a fusion molecule described herein, may be determined as not being a candidate to receive a kinase inhibitor, e.g., a specific kinase inhibitor as described herein.

In another embodiment, responsive to the determination of the presence of the fusion molecule, the subject is identified as likely to respond to a treatment that comprises a kinase inhibitor e.g., a kinase inhibitor as described herein.

In yet another embodiment, responsive to the determination of the presence of the fusion molecule, the method includes administering a kinase inhibitor. e.g., a kinase inhibitor as described herein, to the subject.

### Method of Evaluating a Tumor or a Subject

In another aspect, the invention features a method of evaluating a subject (e.g., a patient), e.g., for risk of having or developing a cancer, e.g., a lung cancer, colorectal cancer or skin cancer. The method includes: acquiring information or knowledge of the presence of a fusion as described herein in a subject (e.g., acquiring genotype information of the subject that identifies a fusion as being present in the subject); acquiring a sequence for a nucleic acid molecule identified herein (e.g., a nucleic acid molecule that includes a fusion molecule sequence described herein); or detecting the presence of a fusion nucleic acid or polypeptide in the subject), wherein the presence of the fusion is positively correlated with increased risk for, or having, a cancer associated with such a fusion.

The method can further include acquiring, e.g., directly or indirectly, a sample from a patient and evaluating the sample for the present of a fusion molecule described herein.

The method can further include the step(s) of identifying (e.g., evaluating, diagnosing, screening, and/or selecting) the subject as being positively correlated with increased risk for, or having, a cancer associated with the fusion molecule.

In another embodiment, a subject identified has having a fusion molecule described herein is identified or selected as likely or unlikely to respond to a treatment, e.g., a kinase inhibitor treatment as described herein. The method can further include treating the subject with a kinase inhibitor, e.g., a kinase inhibitor as described herein.

In certain embodiments, the subject is a patient or patient population that has participated in a clinical trial. In one embodiment, the subject has participated in a clinical trial for evaluating a kinase inhibitor (e.g., a multi-kinase inhibitor or a specific kinase inhibitor). In one embodiment, the clinical trial is discontinued or terminated. In one embodiment, the subject responded favorably to the clinical trial, e.g., experience an improvement in at least one symptom of a cancer (e.g., decreased in tumor size, rate of tumor growth, increased survival). In other embodiments, the subject did not respond in a detectable way to the clinical trial.

In a related aspect, a method of evaluating a patient or a patient population is provided. The method includes: identifying, selecting, or obtaining information or knowledge that the patient or patient population has participated in a clinical trial; acquiring information or knowledge of the presence of a fusion molecule described herein in the patient or patient population (e.g., acquiring genotype information of the subject that identifies a fusion molecule described herein as being present in the subject); acquiring a sequence for a nucleic acid molecule identified herein (e.g., a nucleic acid molecule that includes a fusion sequence); or detecting the presence of a fusion nucleic acid or polypeptide described herein, in the subject), wherein the presence of the fusion identifies the patient or patient population as having an increased risk for, or having, a cancer associated with the fusion molecule.

In some embodiments, the method further includes treating the subject with a kinase inhibitor, e.g., a kinase inhibitor as described herein.

### Reporting

Methods described herein can include providing a report, such as, in electronic, web-based, or paper form, to the patient or to another person or entity, e.g., a caregiver, e.g., a physician, e.g., an oncologist, a hospital, clinic, third-party payor, insurance company or government office. The report can include output from the method, e.g., the identification of nucleotide values, the indication of presence or absence of a fusion molecule described herein, or wild type sequence. In one embodiment, a report is generated, such as in paper or electronic form, which identifies the presence or absence of an alteration described herein, and optionally includes an identifier for the patient from whom the sequence was obtained.

The report can also include information on the role of a fusion molecule described herein, or wild type sequence, in disease. Such information can include information on prognosis, resistance, or potential or suggested therapeutic options. The report can include information on the likely effectiveness of a therapeutic option, the acceptability of a therapeutic option, or the advisability of applying the therapeutic option to a patient, e.g., a patient having a sequence, alteration or mutation identified in the test, and in embodiments, identified in the report. For example, the report can include information, or a recommendation on, the administration of a drug, e.g., the administration at a preselected dosage or in a preselected treatment regimen, e.g., in combination with other drugs, to the patient. In an embodiment, not all mutations identified in the method are identified in the report. For example, the report can be limited to mutations in genes having a preselected level of correlation with the occurrence, prognosis, stage, or susceptibility of the cancer to treatment, e.g., with a preselected therapeutic option. The report can be delivered, e.g., to an entity described herein, within 7, 14, or 21 days from receipt of the sample by the entity practicing the method.

In another aspect, the invention features a method for generating a report, e.g., a personalized cancer treatment report, by obtaining a sample, e.g., a tumor sample, from a subject, detecting a fusion molecule described herein in the sample, and selecting a treatment based on the mutation identified. In one embodiment, a report is generated that annotates the selected treatment, or that lists, e.g., in order of preference, two or more treatment options based on the mutation identified. In another embodiment, the subject, e.g., a patient, is further administered the selected method of treatment.

Unless otherwise defined, all technical and scientific terms used herein have the same meaning as commonly understood by one of ordinary skill in the art to which this invention belongs. Although methods and materials similar or equivalent to those described herein can be used in the practice or testing the invention, suitable methods and materials are described below. All publications, patent applications, patents, and other references mentioned herein are incorporated by reference in their entirety. In case of conflict, the present specification, including definitions, will control. In addition, the materials, methods, and the example are illustrative only and not intended to be limiting.

The details of one or more embodiments featured in the invention are set forth in the accompanying drawings and the description below. Other features, objects, and advantages featured in the invention will be apparent from the description and drawings, and from the claims.

### BRIEF DESCRIPTION OF DRAWINGS

FIGS. 1A-1C are tables summarizing the fusion molecules and the rearrangement events described herein.

FIG. 1A summarizes the following: the name of the fusion (referred to as “fusion”); and the type of rearrangement (referred to as “rearrangement”).

FIG. 1B summarizes the following: the name of the fusion (referred to as “fusion”); the accession number of the full length sequences that contain the 5′- and the 3′-exon sequences (referred to as “5′ Transcript ID” and “3′ Transcript ID,” respectively); and the identity of the last exon of the 5′ transcript and the first exon of the 3′ transcript. The sequences corresponding to the accession numbers provided in FIG. 1B are set forth in the figures appended herein. Alternatively, the sequences can be found by searching the RefSeq Gene as databased at UCSC Genome Browser (genome.ucsc.edu). For example, the following link can be used: http://genome.ucsc.edu/cgi-bin/hgc?hgsid=309144129&c=chr4 &o=1795038&t=1810599&g=reGene&i=NM_{—}000142 to search for Accession Number=NM_{—}000142.

FIG. 1C summarizes the following: the name of the fusion; the SEQ ID NOs. of the nucleotide (Nt) and amino acid (Aa) sequences of the fusion (if shown), the 5′ partner, and the 3′ partner; and the figure in which the sequence is shown. For example, Nt and Aa sequences of the CLIP1-ROS1 fusion have SEQ ID NOs: 13 and 14, respectively. The Nt and Aa sequences of CLIP1 have SEQ ID NOs: 15 and 16, respectively. The Nt and Aa sequences of ROS1 have SEQ ID NOs: 11 and 12, respectively.

FIG. 2 depicts genomic aberrations identified in 38 spitzoid neoplasms by targeted sequencing. (a) The columns denote the samples, the rows denote genes, brown squares represent gene fusions, red squares symbolize point mutations and indels, green squares denote gene amplifications and purple squares indicate truncating mutations. The identified fusion genes and HRAS mutations were mutually exclusive in 38 spitzoid neoplasms. Mutations in PKHD1, ERBB4, LRP1B, and amplifications of MCL1 and CCNE1 are of unknown significance and co-occurred with kinase fusions and HRAS mutations. (b) Illustration of the distinct fusion genes for the ROS1, ALK, NTRK1, RET and BRAF rearrangements. The grey bars represent the exons of the genes, the numbers below the bars the exon number and the blue line the predicted breakpoints. The green shaded areas indicate the kinase domain and the blue shaded areas the coiled-coil domain of the fusion gene product. In the TP53-NTRK1 fusion transcript, multiple breakpoints spanning exon 8-12 of TP53 were predicted.

FIG. 3 depicts ROS1 fusions. (a) Histological section of an atypical Spitz tumor with a PWWP2A ROS1 fusion from the gluteal region of a 55-year-old female (haematoxylin and eosin stain). Scale bar, 500 mm. Scale bar magnification, 50 mm. (b) Immunohistochemistry for ROS shows expression in the melanocytes; stromal cells serve as internal negative controls. Scale bar, 500 mm. Scale bar magnification, 50 mm. The FISH inset confirms the gene rearrangements using breakpoint flanking FISH probes. The rearranged ROS1 locus appears as individual green and red signals, and the wild-type kinase allele with juxtaposed green/red signals. Scale bar, 10 mm. (c) Illustration of the PWWP2A-ROS1 kinase fusion. ROS1 is located on chromosome 1 q21, and PWWP2A on chromosome 5q33. Owing to genomic rearrangements, exon 1 of PWWP2A is fused with exon 36-43 of ROS1, which contains the tyrosine kinase domain. The in-frame fusion junction of the transcript was confirmed by Sanger sequencing. (d) The PWWP2A-ROS1 fusion, but not the control-GFP construct, induces p-AKT, p-ERK, p-S6 and p-SHP2 in melan-a cells. Crizotinib inhibited at least partially the phosphorylation of the chimeric PWWP2A-ROS1 fusion protein, p-AKT, p-S6 and p-SHP2. The indicated protein weight markers in kDa are estimated from molecular weight standards. Results are representative of three independent experiments.

FIG. 4 depicts ALK fusions. (a) Histologic section of an atypical Spitz tumor excised from the upper arm of a 19-year-old male with a DCTN1-ALK fusion (hematoxylin and eosin stain). Scale bar, 500 μm. Scale bar magnification, 50 μm. (b) Immunohistochemistry shows ALK expression in the melanocytes; stromal cells serve as internal negative controls. Scale bar, 500 μm. Scale bar magnification, 50 μm. (c) FISH demonstrates the ALK gene rearrangement by the individual green and orange signals using breakpoint flanking probes. Scale bar, 10 μm. (d) Illustration of the DCTN1-ALK kinase fusion. ALK is located on chromosome 1p23 and DCTN1 on chromosome 2p13. Due to genomic rearrangements, exon 1-26 of DCTN1 is fused with exon 20 to 29 of ALK, which contains the tyrosine kinase domain. The in-frame junction of the fusion transcript was confirmed with Sanger sequencing. (e) The DCTN1-ALK fusion construct, but not the control-GFP construct, induces p-AKT, p-ERK and p-S6 in melan-a cells. These effects and the phosphorylation of chimeric DCTN1-ALK protein can be inhibited with crizotinib. The indicated protein weight markers in kDa are estimated from molecular weight standards. Results are representative of three independent experiments.

FIG. 5 depicts NTRK1 fusions. (a) Histologic section of a spitzoid melanoma excised from the left knee of a 39-year-old woman with an LMNA-NTRK1 fusion (hematoxylin and eosin stain). Scale bar, 500 μm. Scale bar magnification, 50 μm. (b) Immunohistochemistry demonstrates the NTRK1 expression in melanocytes; stromal cells serve as internal negative controls. Scale bar, 500 μm. Scale bar magnification, 50 μm. The FISH inset confirms the gene rearrangements using breakpoint flanking FISH probes by the individual green and red signals. Scale bar, 10 μm. (c) The LMNA-NTRK1 kinase fusion is caused by a 743 kb deletion on chromosome 1q, joining the first 2 exons of LMNA with exon 11 to 17 of NTRK1. The in-frame junction of the fusion transcript was confirmed with Sanger sequencing. (d) The LMNA-NTRK1 fusion construct, but not the full-length, wild-type NTRK1 or the control-GFP constructs induce p-AKT, p-ERK, pS6 and p-PLCγ1 in melan-a cells. A small molecule kinase inhibitor, AZ-23, inhibited the phosphorylation of LMNA-NTRK1 and the activation of the oncogenic signaling pathways. The indicated protein weight markers in kDa are estimated from molecular weight standards. Results are representative of three independent experiments.

FIG. 6 depicts RET fusions. (a) Histologic section of a pigmented spindle cell nevus (a morphologic variant of Spitz nevus) from a 50-year-old woman with a GOLGA5-RET fusion (hematoxylin and eosin stain). Scale bar, 500 μm. Scale bar magnification, 50 μm. (b) RET expression in melanocytes; keratinocytes serve as internal negative controls. Scale bar, 100 μm. The individual green and red signals in FISH confirm the gene rearrangements using breakpoint flanking FISH probes. Scale bar, 10 μm. (c, d) The GOLGA5-RET construct, but not the wild-type, full-length RET or the control-GFP constructs, induces p-AKT, p-ERK, p-S6, and p-PLCγ1 in melan-a cells. The activation of these pathways and the phosphorylation of GOLGA5-RET can be inhibited with (c) vandetanib and (d) cabozantinib. The indicated protein weight markers in kDa are estimated from molecular weight standards. Results are representative of three independent experiments.

FIG. 7 depicts in vivo tumorigenic potential of kinase fusion in melanocytic cells. All tested kinase fusions stably expressed in melan-a cells formed tumors in NOD/SCID/interleukin 2 receptor γ null mice within 40 days. As expected melan-a stably transduced with NRASG12V and HRASG12V also formed tumors while control GFP did not.

FIG. 8 depicts full western blots of FIG. 3d. (a) p-ROS1, (b) t-ROS1, (c) p-AKT, (d) t-AKT, (e) p-ERK, (f) t-ERK, (g) p-S6, (h) t-S6, (i) p-SHP2, (j) t-SHP2, (k) Hsp60. All molecular weight standards are indicated in kDa.

FIG. 9 depicts full western blots of FIG. 4e. (a) p-ALK, (b) t-ALK, (c) p-AKT, (d) t-AKT, (e) p-ERK, (f) t-ERK, (g) p-S6, (h) t-S6, (i) Hsp60. All molecular weight standards are indicated in kDa.

FIG. 10 depicts full western blots of FIG. 5d. (a) p-NTRK1, (b) t-NTRK1, (c) p-AKT, (d) t-AKT, (e) p-ERK, (f) t-ERK, (g) p-S6, (h) t-S6, (i) p-PLCγ1, (j) t-PLCγ1, (k) Hsp60. All molecular weight standards are indicated in kDa.

FIG. 11 depicts full western blots of FIG. 6c. (a) p-RET, (b) t-RET, (c) p-AKT (d) t-AKT and p-ERK, (e) t-ERK, (f) p-S6, (g) t-S6, (h) p-PLCγ1, (i) t-PLCγ1, (j) Hsp60. All molecular weight standards are indicated in kDa.

FIG. 12 depicts full western blots of FIG. 6d. (a) p-RET, (b) t-RET, (c) p-AKT and p-ERK (lower, overexposed band), (d) t-AKT, (e) p-ERK, (f) t-ERK, (g) p-S6, (h) t-S6, (i) p-PLCγ1, (j) t-PLCγ1, (k) Hsp60. All molecular weight standards are indicated in kDa.

### DETAILED DESCRIPTION

The invention is based, at least in part, on the discovery of novel fusion events, and their association with cancer.

Certain terms are first defined. Additional terms are defined throughout the specification.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

“Acquire” or “acquiring” as the terms are used herein, refer to obtaining possession of a physical entity, or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance. e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

“Acquiring a sequence” as the term is used herein, refers to obtaining possession of a nucleotide sequence or amino acid sequence, by “directly acquiring” or “indirectly acquiring” the sequence. “Directly acquiring a sequence” means performing a process (e.g., performing a synthetic or analytical method) to obtain the sequence, such as performing a sequencing method (e.g., a Next Generation Sequencing (NGS) method). “Indirectly acquiring a sequence” refers to receiving information or knowledge of, or receiving, the sequence from another party or source (e.g., a third party laboratory that directly acquired the sequence). The sequence acquired need not be a full sequence, e.g., sequencing of at least one nucleotide, or obtaining information or knowledge that identifies a fusion molecule disclosed herein as being present in a subject constitutes acquiring a sequence.

Directly acquiring a sequence includes performing a process that includes a physical change in a physical substance, e.g., a starting material, such as a tissue sample, e.g., a biopsy, or an isolated nucleic acid (e.g., DNA or RNA) sample. Exemplary changes include making a physical entity from two or more starting materials, shearing or fragmenting a substance, such as a genomic DNA fragment; separating or purifying a substance (e.g., isolating a nucleic acid sample from a tissue); combining two or more separate entities into a mixture, performing a chemical reaction that includes breaking or forming a covalent or non-covalent bond. Directly acquiring a value includes performing a process that includes a physical change in a sample or another substance as described above.

“Acquiring a sample” as the term is used herein, refers to obtaining possession of a sample, e.g., a tissue sample or nucleic acid sample, by “directly acquiring” or “indirectly acquiring” the sample. “Directly acquiring a sample” means performing a process (e.g., performing a physical method such as a surgery or extraction) to obtain the sample. “Indirectly acquiring a sample” refers to receiving the sample from another party or source (e.g., a third party laboratory that directly acquired the sample). Directly acquiring a sample includes performing a process that includes a physical change in a physical substance, e.g., a starting material, such as a tissue, e.g., a tissue in a human patient or a tissue that has was previously isolated from a patient. Exemplary changes include making a physical entity from a starting material, dissecting or scraping a tissue; separating or purifying a substance (e.g., a sample tissue or a nucleic acid sample); combining two or more separate entities into a mixture; performing a chemical reaction that includes breaking or forming a covalent or non-covalent bond. Directly acquiring a sample includes performing a process that includes a physical change in a sample or another substance, e.g., as described above.

“Binding entity” means any molecule to which molecular tags can be directly or indirectly attached that is capable of specifically binding to an analyte. The binding entity can be an affinity tag on a nucleic acid sequence. In certain embodiments, the binding entity allows for separation of the nucleic acid from a mixture, such as an avidin molecule, or an antibody that binds to the hapten or an antigen-binding fragment thereof. Exemplary binding entities include, but are not limited to, a biotin molecule, a hapten, an antibody, an antibody binding fragment, a peptide, and a protein.

“Complementary” refers to sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In certain embodiments, the first region comprises a first portion and the second region comprises a second portion, whereby, when the first and second portions are arranged in an antiparallel fashion, at least about 50%, at least about 75%, at least about 90%, or at least about 95% of the nucleotide residues of the first portion are capable of base pairing with nucleotide residues in the second portion. In other embodiments, all nucleotide residues of the first portion are capable of base pairing with nucleotide residues in the second portion.

The term “cancer” or “tumor” is used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers. In certain embodiments, the cancer is a solid tumor, a soft tissue tumor, or a metastatic lesion.

The term “neoplasm” or “neoplastic” cell refers to an abnormal proliferative stage, e.g., a hyperproliferative stage, in a cell or tissue that can include a benign, pre-malignant, malignant (cancer) or metastatic stage.

The term “fusion” or “fusion molecule” can refer to a polypeptide or a nucleic acid fusion, depending on the context.

The terms “Spitz neoplasia”, “Spitz neoplasm”, “Spitz tumor”, “Spitz lesion”, “Spitzoid neoplasia”, or “Spitzoid neoplasm”, or “Spitzoid tumor”, or “Spitzoid lesion” are used interchangeably herein. These terms refer to a neoplasm belonging to one of three categories (1) Spitz nevi, or tumors without appreciable abnormality, (2) Spitz tumors with one or more atypical features (atypical Spitz tumor) including those with indeterminate biological malignant potential, and (3) malignant melanoma with histopathological features reminiscent of a Spitz nevus such as large epithelioid or spindled melanocytes (See e.g., Barnhill, R. L. et al., Modern Pathology (2006) 19, S21-S33).

Cancer is “inhibited” if at least one symptom of the cancer is alleviated, terminated, slowed, or prevented. As used herein, cancer is also “inhibited” if recurrence or metastasis of the cancer is reduced, slowed, delayed, or prevented.

“Chemotherapeutic agent” means a chemical substance, such as a cytotoxic or cytostatic agent, that is used to treat a condition, particularly cancer.

As used herein, “cancer therapy” and “cancer treatment” are synonymous terms.

As used herein, “chemotherapy” and “chemotherapeutic” and “chemotherapeutic agent” are synonymous terms.

The terms “homology” or “identity,” as used interchangeably herein, refer to sequence similarity between two polynucleotide sequences or between two polypeptide sequences, with identity being a more strict comparison. The phrases “percent identity or homology” and “% identity or homology” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value there between. Identity or similarity can be determined by comparing a position in each sequence that can be aligned for purposes of comparison. When a position in the compared sequence is occupied by the same nucleotide base or amino acid, then the molecules are identical at that position. A degree of similarity or identity between polynucleotide sequences is a function of the number of identical or matching nucleotides at positions shared by the polynucleotide sequences. A degree of identity of polypeptide sequences is a function of the number of identical amino acids at positions shared by the polypeptide sequences. A degree of homology or similarity of polypeptide sequences is a function of the number of amino acids at positions shared by the polypeptide sequences. The term “substantially identical,” as used herein, refers to an identity or homology of at least 75%, at least 80%, at least 85%, at least 90%, 91%, 92%, 93%, 94%, 95%, 96%, 97%, 98%, 99%, or more.

“Likely to” or “increased likelihood,” as used herein, refers to an increased probability that an item, object, thing or person will occur. Thus, in one example, a subject that is likely to respond to treatment with a kinase inhibitor, alone or in combination, has an increased probability of responding to treatment with the inhibitor alone or in combination, relative to a reference subject or group of subjects.

“Unlikely to” refers to a decreased probability that an event, item, object, thing or person will occur with respect to a reference. Thus, a subject that is unlikely to respond to treatment with a kinase inhibitor, alone or in combination, has a decreased probability of responding to treatment with a kinase inhibitor, alone or in combination, relative to a reference subject or group of subjects.

“Sequencing” a nucleic acid molecule requires determining the identity of at least 1 nucleotide in the molecule. In embodiments, the identities of less than all of the nucleotides in a molecule are determined. In other embodiments, the identities of a majority or all of the nucleotides in the molecule are determined.

“Next-generation sequencing or next-gen sequencing or NGS or NG sequencing” as used herein, refers to any sequencing method that determines the nucleotide sequence of either individual nucleic acid molecules (e.g., in single molecule sequencing) or clonally expanded proxies for individual nucleic acid molecules in a highly parallel fashion (e.g., greater than 10⁵ molecules are sequenced simultaneously). In one embodiment, the relative abundance of the nucleic acid species in the library can be estimated by counting the relative number of occurrences of their cognate sequences in the data generated by the sequencing experiment. Next generation sequencing methods are known in the art, and are described, e.g., in Metzker, M. (2010) Nature Biotechnology Reviews 11:31-46, incorporated herein by reference. Next generation sequencing can detect a variant present in less than 5% of the nucleic acids in a sample.

“Sample,” “tissue sample,” “patient sample,” “patient cell or tissue sample” or “specimen” each refers to a collection of similar cells obtained from a tissue of a subject or patient. The source of the tissue sample can be solid tissue as from a fresh, frozen and/or preserved organ, tissue sample, biopsy, or aspirate; blood or any blood constituents; bodily fluids such as cerebral spinal fluid, amniotic fluid, peritoneal fluid or interstitial fluid; or cells from any time in gestation or development of the subject. The tissue sample can contain compounds that are not naturally intermixed with the tissue in nature such as preservatives, anticoagulants, buffers, fixatives, nutrients, antibiotics or the like. In one embodiment, the sample is preserved as a frozen sample or as formaldehyde- or paraformaldehyde-fixed paraffin-embedded (FFPE) tissue preparation. For example, the sample can be embedded in a matrix, e.g., an FFPE block or a frozen sample.

A “tumor nucleic acid sample” as used herein, refers to nucleic acid molecules from a tumor or cancer sample. Typically, it is DNA, e.g., genomic DNA, or cDNA derived from RNA, from a tumor or cancer sample. In certain embodiments, the tumor nucleic acid sample is purified or isolated (e.g., it is removed from its natural state).

A “control” or “reference” “nucleic acid sample” as used herein, refers to nucleic acid molecules from a control or reference sample. Typically, it is DNA, e.g., genomic DNA, or cDNA derived from RNA, not containing the alteration or variation in the gene or gene product, e.g., not containing a fusion molecule described herein. In certain embodiments, the reference or control nucleic acid sample is a wild type or a non-mutated sequence. In certain embodiments, the reference nucleic acid sample is purified or isolated (e.g., it is removed from its natural state). In other embodiments, the reference nucleic acid sample is from a non-tumor sample, e.g., a blood control, a normal adjacent tumor (NAT), or any other non-cancerous sample from the same or a different subject.

“Adjacent to the interrogation position,” as used herein, means that a site sufficiently close such that a detection reagent complementary with the site can be used to distinguish between a mutation, e.g., a mutation described herein, and a reference sequence, e.g., a non-mutant or wild-type sequence, in a target nucleic acid. Directly adjacent, as used herein, is where 2 nucleotides have no intervening nucleotides between them.

“Associated mutation,” as used herein, refers to a mutation within a preselected distance, in terms of nucleotide or primary amino acid sequence, from a definitional mutation, e.g., a mutant as described herein, e.g., a translocation, breakpoint or fusion molecule described herein. In embodiments, the associated mutation is within n, wherein n is 2, 5, 10, 20, 30, 50, 100, or 200 nucleotides from the definitional mutation (n does not include the nucleotides defining the associated and definitional mutations). In embodiments, the associated mutation is a translocation mutation.

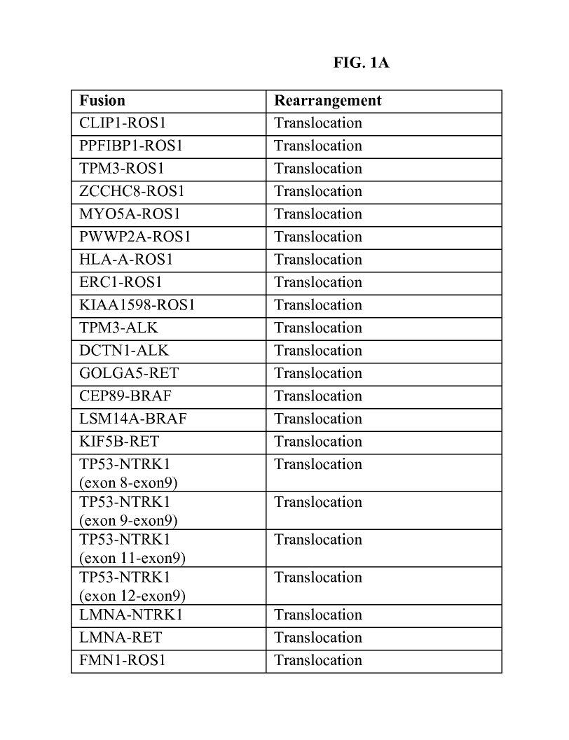

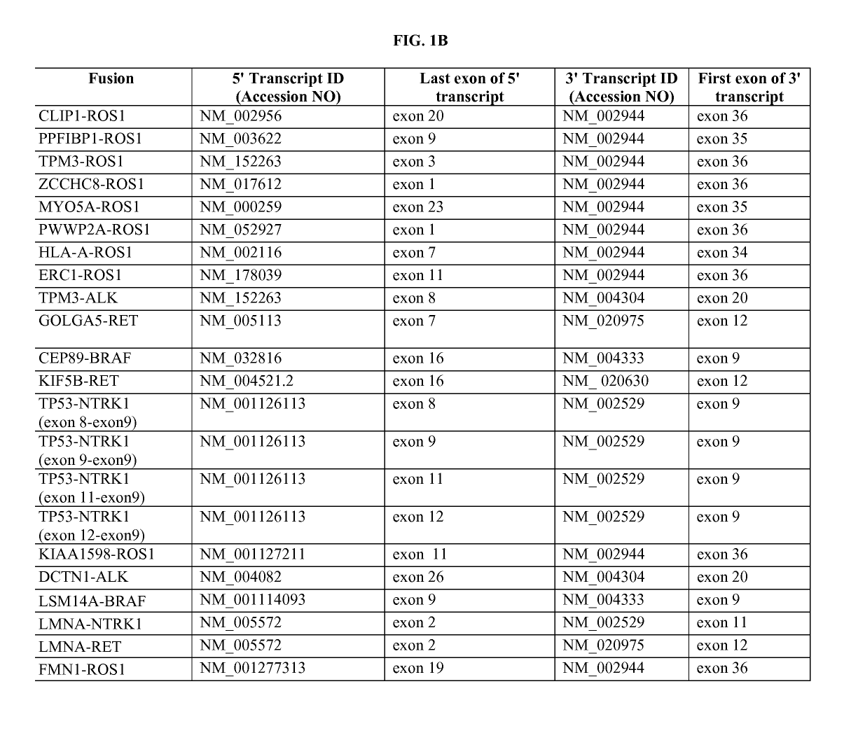

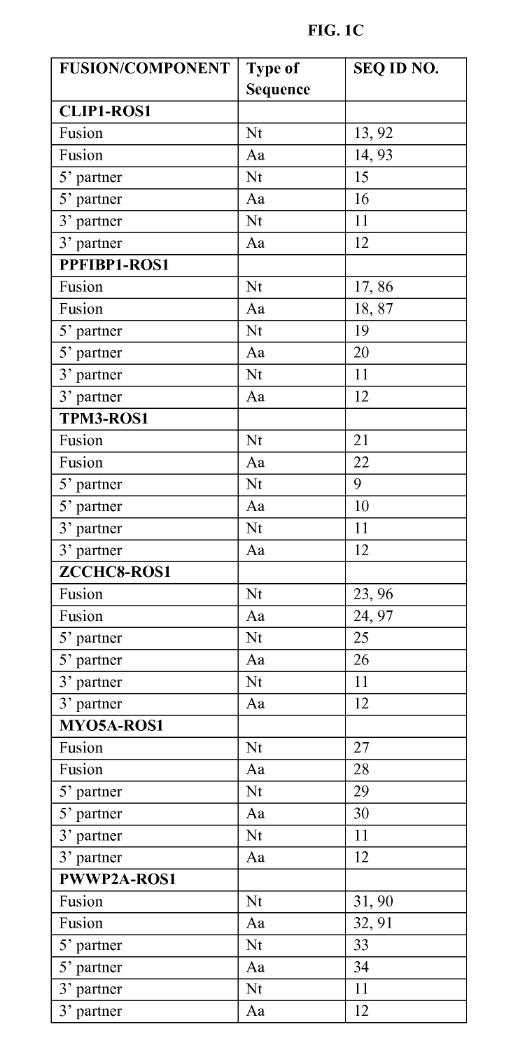

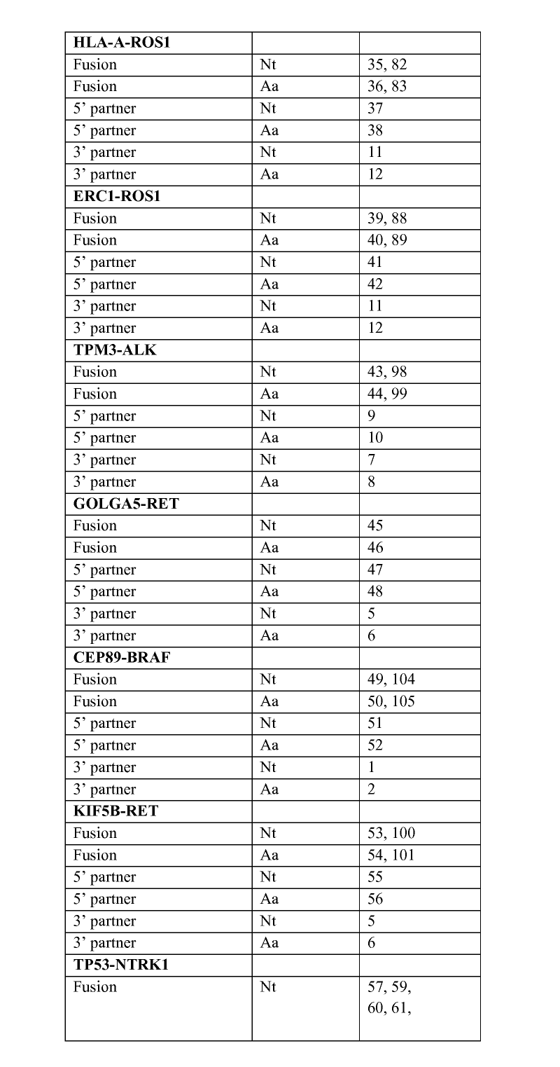

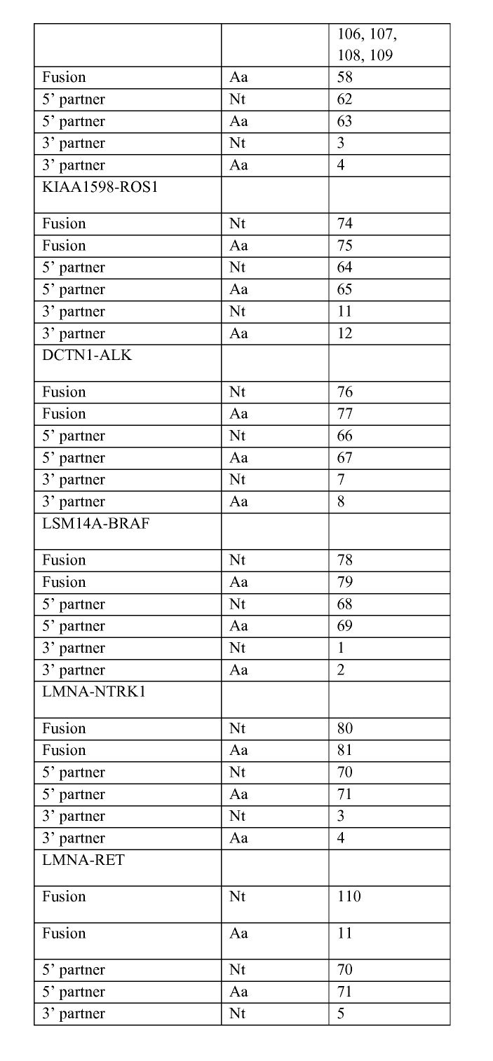

“Interrogation position,” as used herein, comprises at least one nucleotide (or, in the case of polypeptides, an amino acid residue) which corresponds to a nucleotide (or amino acid residue) that is mutated in a mutation, including, e.g., in the case of a rearrangement, one or both of the nucleotides (or amino acid residues) flanking the breakpoint, or other residue which can be used to distinguish the mutation, of interest, e.g., a mutation being identified, or in a nucleic acid (or protein) being analyzed, e.g., sequenced, or recovered. By way of example, the interrogation position in the breakpoint shown in FIG. 1A, 1B or 1C, includes one, two, or more nucleotide positions at the junction site.

A “reference sequence,” as used herein, e.g., as a comparator for a mutant sequence, is a sequence which has a different nucleotide or amino acid at an interrogation position than does the mutant(s) being analyzed. In an embodiment, the reference sequence is wild-type for at least the interrogation position.

Headings, e.g., (a), (b), (i) etc., are presented merely for ease of reading the specification and claims. The use of headings in the specification or claims does not require the steps or elements be performed in alphabetical or numerical order or the order in which they are presented.

Various aspects featured in the invention are described in further detail below. Additional definitions are set out throughout the specification.

### Isolated Nucleic Acid Molecules

One aspect of the present disclosure pertains to isolated nucleic acid molecules that include a fusion molecule described herein, including nucleic acids which encode a fusion polypeptide or a portion of such a polypeptide. The nucleic acid molecules include those nucleic acid molecules which reside in genomic regions identified herein. As used herein, the term “nucleic acid molecule” includes DNA molecules (e.g., cDNA or genomic DNA) and RNA molecules (e.g., mRNA) and analogs of the DNA or RNA generated using nucleotide analogs. The nucleic acid molecule can be single-stranded or double-stranded; in certain embodiments the nucleic acid molecule is double-stranded DNA.

Isolated nucleic acid molecules also include nucleic acid molecules sufficient for use as hybridization probes or primers to identify nucleic acid molecules that correspond to a fusion molecule described herein, e.g., those suitable for use as PCR primers for the amplification or mutation of nucleic acid molecules.

An “isolated” nucleic acid molecule is one which is separated from other nucleic acid molecules which are present in the natural source of the nucleic acid molecule. In certain embodiments, an “isolated” nucleic acid molecule is free of sequences (such as protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, less than about 4 kB, less than about 3 kB, less than about 2 kB, less than about 1 kB, less than about 0.5 kB or less than about 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

The language “substantially free of other cellular material or culture medium” includes preparations of nucleic acid molecule in which the molecule is separated from cellular components of the cells from which it is isolated or recombinantly produced. Thus, nucleic acid molecule that is substantially free of cellular material includes preparations of nucleic acid molecule having less than about 30%, less than about 20%, less than about 10%, or less than about 5% (by dry weight) of other cellular material or culture medium.

A fusion nucleic acid molecule can be isolated using standard molecular biology techniques and the sequence information in the database records described herein. Using all or a portion of such nucleic acid sequences, fusion nucleic acid molecules as described herein can be isolated using standard hybridization and cloning techniques (e.g., as described in Sambrook et al., ed., Molecular Cloning: A Laboratory Manual, 2nd ed. Cold Spring Harbor Laboratory Press, Cold Spring Harbor, N. Y., 1989).

A fusion nucleic acid molecule (e.g., fusion molecule described herein) can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, oligonucleotides corresponding to all or a portion of a nucleic acid molecule featured in the invention can be prepared by standard synthetic techniques, e.g., using an automated DNA synthesizer.

In another embodiment, a fusion nucleic acid molecule (e.g., fusion molecule described herein) comprises a nucleic acid molecule which has a nucleotide sequence complementary to the nucleotide sequence of the fusion nucleic acid molecule or to the nucleotide sequence of a nucleic acid encoding a fusion protein. A nucleic acid molecule which is complementary to a given nucleotide sequence is one which is sufficiently complementary to the given nucleotide sequence that it can hybridize to the given nucleotide sequence thereby forming a stable duplex.

Moreover, a fusion nucleic acid molecule can comprise only a portion of a nucleic acid sequence, wherein the full length nucleic acid sequence or which encodes a fusion polypeptide. Such nucleic acid molecules can be used, for example, as a probe or primer. The probe/primer typically is used as one or more substantially purified oligonucleotides. The oligonucleotide typically comprises a region of nucleotide sequence that hybridizes under stringent conditions to at least about 7, at least about 15, at least about 25, at least about 50, at least about 75, at least about 100, at least about 125, at least about 150, at least about 175, at least about 200, at least about 250, at least about 300, at least about 350, at least about 400, at least about 500, at least about 600, at least about 700, at least about 800, at least about 900, at least about 1 kb, at least about 2 kb, at least about 3 kb, at least about 4 kb, at least about 5 kb, at least about 6 kb, at least about 7 kb, at least about 8 kb, at least about 9 kb, at least about 10 kb, at least about 15 kb, at least about 20 kb, at least about 25 kb, at least about 30 kb, at least about 35 kb, at least about 40 kb, at least about 45 kb, at least about 50 kb, at least about 60 kb, at least about 70 kb, at least about 80 kb, at least about 90 kb, at least about 100 kb, at least about 200 kb, at least about 300 kb, at least about 400 kb, at least about 500 kb, at least about 600 kb, at least about 700 kb, at least about 800 kb, at least about 900 kb, at least about 1 mb, at least about 2 mb, at least about 3 mb, at least about 4 mb, at least about 5 mb, at least about 6 mb, at least about 7 mb, at least about 8 mb, at least about 9 mb, at least about 10 mb or more consecutive nucleotides of a fusion nucleic acid described herein.

The disclosure further describes nucleic acid molecules that are substantially identical to the gene mutations and/or gene products described herein, such that they are at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or greater. The disclosure further describes nucleic acid molecules that are substantially identical to the gene mutations and/or gene products described herein, such that they are at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or greater.

In other embodiments, the invention further encompasses nucleic acid molecules that are substantially homologous to fusion gene mutations and/or gene products described herein, such that they differ by only or at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, at least 500, at least 600 nucleotides or any range in between.

In another embodiment, an isolated fusion nucleic acid molecule described herein is at least 7, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 250, at least 300, at least 350, at least 400, at least 450, at least 550, at least 650, at least 700, at least 800, at least 900, at least 1000, at least 1200, at least 1400, at least 1600, at least 1800, at least 2000, at least 2200, at least 2400, at least 2600, at least 2800, at least 3000, or more nucleotides in length and hybridizes under stringent conditions to a fusion nucleic acid molecule or to a nucleic acid molecule encoding a protein corresponding to a marker featured in the invention.

As used herein, the term “hybridizes under stringent conditions” is intended to describe conditions for hybridization and washing under which nucleotide sequences at least 60%, at least 65%, at least 70%, at least 75%, at least 80%, or at least 85% identical to each other typically remain hybridized to each other. Such stringent conditions are known to those skilled in the art and can be found in sections 6.3.1-6.3.6 of Current Protocols in Molecular Biology, John Wiley & Sons, N.Y. (1989), incorporated herein by reference. Another, non-limiting example of stringent hybridization conditions are hybridization in 6× sodium chloride/sodium citrate (SSC) at about 45° C., followed by one or more washes in 0.2×SSC, 0.1% SDS at 50-65° C.

The disclosure is also directed to molecular beacon nucleic acid molecules having at least one region which is complementary to a fusion nucleic acid molecule described herein, such that the molecular beacon is useful for quantitating the presence of the nucleic acid molecule featured in the invention in a sample. A “molecular beacon” nucleic acid is a nucleic acid molecule comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid molecules are not annealed with one another, fluorescence of the fluorophore is quenched to a lesser degree. Molecular beacon nucleic acid molecules are described, for example, in U.S. Pat. No. 5,876,930, incorporated herein by reference.

### Probes

The invention also provides isolated nucleic acid molecules useful as probes. Such nucleic acid probes can be designed based on the sequence of a fusion molecule described herein.

Probes based on the sequence of a fusion nucleic acid molecule as described herein can be used to detect transcripts or genomic sequences corresponding to one or more markers featured in the invention. The probe comprises a label group attached thereto, e.g., a radioisotope, a fluorescent compound, an enzyme, or an enzyme co-factor. Such probes can be used as part of a test kit for identifying cells or tissues which express the fusion protein (e.g., a fusion described herein), such as by measuring levels of a nucleic acid molecule encoding the protein in a sample of cells from a subject, e.g., detecting mRNA levels or determining whether a gene encoding the protein has been mutated or deleted.

Probes featured in the invention include those that will specifically hybridize to a gene sequence described in the Examples, e.g., fusion molecule described herein. Typically these probes are 12 to 20. e.g., 17 to 20 nucleotides in length (longer for large insertions) and have the nucleotide sequence corresponding to the region of the mutations at their respective nucleotide locations on the gene sequence. Such molecules can be labeled according to any technique known in the art, such as with radiolabels, fluorescent labels, enzymatic labels, sequence tags, biotin, other ligands, etc. As used herein, a probe that “specifically hybridizes” to a fusion gene sequence will hybridize under high stringency conditions.

A probe will typically contain one or more of the specific mutations described herein. Typically, a nucleic acid probe will encompass only one mutation. Such molecules may be labeled and can be used as allele-specific probes to detect the mutation of interest.

In one aspect, the invention features a probe or probe set that specifically hybridizes to a nucleic acid comprising an inversion resulting in a fusion molecule described herein. In another aspect, the invention features a probe or probe set that specifically hybridizes to a nucleic acid comprising a deletions resulting in a fusion molecule described herein.

Isolated pairs of allele specific oligonucleotide probes are also provided, where the first probe of the pair specifically hybridizes to the mutant allele, and the second probe of the pair specifically hybridizes to the wild type allele. For example, in one exemplary probe pair, one probe will recognize the fusion junction in the CEP89-BRAF fusion, and the other probe will recognize a sequence downstream or upstream of CEP89 or BRAF, neither of which includes the fusion junction. These allele-specific probes are useful in detecting a BRAF somatic mutation in a tumor sample, e.g., lung adenocarcinoma sample. In a similar manner, probe pairs can be designed and produced for any of the fusion molecule described herein, and are useful in detecting an somatic mutation in a tumor sample.

### Primers

The invention also provides isolated nucleic acid molecules useful as primers.

The term “primer” as used herein refers to a sequence comprising two or more deoxyribonucleotides or ribonucleotides, e.g., more than three, and more than eight, or at least 20 nucleotides of a gene described in the Example, where the sequence corresponds to a sequence flanking one of the mutations or a wild type sequence of a gene identified in the Example, e.g., any gene described herein involved in a fusion described herein. Primers may be used to initiate DNA synthesis via the PCR (polymerase chain reaction) or a sequencing method. Primers featured in the invention include the sequences recited and complementary sequences which would anneal to the opposite DNA strand of the sample target. Since both strands of DNA are complementary and mirror images of each other, the same segment of DNA will be amplified.

Primers can be used to sequence a nucleic acid, e.g., an isolated nucleic acid described herein, such as by an NGS method, or to amplify a gene described in the Examples, such as by PCR. The primers can specifically hybridize, for example, to the ends of the exons or to the introns flanking the exons. The amplified segment can then be further analyzed for the presence of the mutation such as by a sequencing method. The primers are useful in directing amplification of a target polynucleotide prior to sequencing. In another aspect, the present disclosure features a pair of oligonucleotide primers that amplify a region that contains or is adjacent to a fusion junction identified in the Examples. Such primers are useful in directing amplification of a target region that includes a fusion junction identified in the Example, e.g., prior to sequencing. The primer typically contains 12 to 20, or 17 to 20, or more nucleotides, although a primer may contain fewer nucleotides.

A primer is typically single stranded, e.g., for use in sequencing or amplification methods, but may be double stranded. If double stranded, the primer may first be treated to separate its strands before being used to prepare extension products. A primer must be sufficiently long to prime the synthesis of extension products in the presence of the inducing agent for polymerization. The exact length of primer will depend on many factors, including applications (e.g., amplification method), temperature, buffer, and nucleotide composition. A primer typically contains 12-20 or more nucleotides, although a primer may contain fewer nucleotides.

Primers are typically designed to be “substantially” complementary to each strand of a genomic locus to be amplified. Thus, the primers must be sufficiently complementary to specifically hybridize with their respective strands under conditions which allow the agent for polymerization to perform. In other words, the primers should have sufficient complementarity with the 5′ and 3′ sequences flanking the mutation to hybridize therewith and permit amplification of the genomic locus.

The term “substantially complementary to” or “substantially the sequence” refers to sequences that hybridize to the sequences provided under stringent conditions and/or sequences having sufficient homology with a sequence comprising a fusion junction identified in the Example, or the wild type counterpart sequence, such that the allele specific oligonucleotides hybridize to the sequence. In one embodiment, a sequence is substantially complementary to a fusion junction in an inversion event, e.g., to a fusion junction in any fusion molecule described herein. “Substantially the same” as it refers to oligonucleotide sequences also refers to the functional ability to hybridize or anneal with sufficient specificity to distinguish between the presence or absence of the mutation. This is measurable by the temperature of melting being sufficiently different to permit easy identification of whether the oligonucleotide is binding to the normal or mutant gene sequence identified in the Example.

In one aspect, the invention features a primer or primer set for amplifying a nucleic acid comprising an inversion resulting in a fusion described herein. In another aspect, the invention features a primer or primer set for amplifying a nucleic acid comprising a deletion resulting in fusion described herein.

Isolated pairs of allele specific oligonucleotide primer are also provided, where the first primer of the pair specifically hybridizes to the mutant allele, and the second primer of the pair specifically hybridizes to a sequence upstream or downstream of a mutation, or a fusion junction resulting from, e.g., an inversion, duplication, deletion, insertion or translocation. In one exemplary primer pair, one probe will recognize a CEP89-BRAF fusion, such as by hybridizing to a sequence at the fusion junction between the CEP89 and BRAF transcripts, and the other primer will recognize a sequence upstream or downstream of the fusion junction. These allele-specific primers are useful for amplifying a CEP89-BRAF fusion sequence from a tumor sample, e.g., a biopsy, e.g., a biopsy from a suspected cancer.

In another exemplary primer pair, one primer can recognize an CEP89-BRAF translocation (e.g., the reciprocal of the CEP89-BRAF translocation), such as by hybridizing to a sequence at the fusion junction between the CEP89 and BRAF transcripts, and the other primer will recognize a sequence upstream or downstream of the fusion junction. These allele-specific primers are useful for amplifying a CEP89-BRAF fusion sequence from a tumor sample, e.g., a cancer sample or biopsy or biopsy sample.

Primers can be prepared using any suitable method, such as conventional phosphotriester and phosphodiester methods or automated embodiments thereof. In one such automated embodiment, diethylphosphoramidites are used as starting materials and may be synthesized as described by Beaucage, et al., Tetrahedron Letters, 22:1859-1862, (1981), incorporated herein by reference. One method for synthesizing oligonucleotides on a modified solid support is described in U.S. Pat. No. 4,458,066, incorporated herein by reference.

An oligonucleotide probe or primer that hybridizes to a mutant or wild type allele is said to be the complement of the allele. As used herein, a probe exhibits “complete complementarity” when every nucleotide of the probe is complementary to the corresponding nucleotide of the allele. Two polynucleotides are said to be “minimally complementary” if they can hybridize to one another with sufficient stability to permit them to remain annealed to one another under at least conventional “low-stringency” conditions. Similarly, the polynucleotides are said to be “complementary” if they can hybridize to one another with sufficient stability to permit them to remain annealed to one another under conventional “high-stringency” conditions. Conventional stringency conditions are known to those skilled in the art and can be found, for example in Molecular Cloning: A Laboratory Manual, 3rd edition Volumes 1, 2, and 3. J. F. Sambrook, D. W. Russell, and N. Irwin, Cold Spring Harbor Laboratory Press, 2000, incorporated herein by reference.

Departures from complete complementarity are therefore permissible, as long as such departures do not completely preclude the capacity of a probe to hybridize to an allele. Thus, in order for a polynucleotide to serve as a primer or probe it need only be sufficiently complementary in sequence to be able to form a stable double-stranded structure under the particular solvent and salt concentrations employed. Appropriate stringency conditions which promote DNA hybridization are, for example, 6.0× sodium chloride/sodium citrate (SSC) at about 45° C., followed by a wash of 2.0×SSC at 50° C. Such conditions are known to those skilled in the art and can be found, for example in Current Protocols in Molecular Biology, John Wiley & Sons, N.Y. (1989), incorporated herein by reference. Salt concentration and temperature in the wash step can be adjusted to alter hybridization stringency. For example, conditions may vary from low stringency of about 2.0×SSC at 40° C. to moderately stringent conditions of about 2.0×SSC at 50° C. to high stringency conditions of about 0.2×SSC at 50° C.

### Fusion Proteins and Antibodies

One aspect featured in the invention pertains to purified fusion polypeptides, and biologically active portions thereof. The fusion polypeptide can be any fusion molecule described herein. In one embodiment, the native fusion polypeptide can be isolated from cells or tissue sources by an appropriate purification scheme using standard protein purification techniques. In another embodiment, a fusion polypeptide is produced by recombinant DNA techniques. Alternative to recombinant expression, a fusion polypeptide described herein can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, less than about 20%, less than about 10%, or less than about 5% (by dry weight) of heterologous protein (also referred to herein as a “contaminating protein”). When the protein or biologically active portion thereof is recombinantly produced, it can be substantially free of culture medium, i.e., culture medium represents less than about 20%, less than about 10%, or less than about 5% of the volume of the protein preparation. When the protein is produced by chemical synthesis, it can substantially be free of chemical precursors or other chemicals, i.e., it is separated from chemical precursors or other chemicals which are involved in the synthesis of the protein. Accordingly such preparations of the protein have less than about 30%, less than about 20%, less than about 10%, less than about 5% (by dry weight) of chemical precursors or compounds other than the polypeptide of interest.

Biologically active portions of a fusion polypeptide include polypeptides comprising amino acid sequences sufficiently identical to or derived from the amino acid sequence of the fusion protein, which include fewer amino acids than the full length protein, and exhibit at least one activity of the corresponding full-length protein, e.g., a kinase activity e.g., a BRAF kinase activity. A biologically active portion of a protein featured in the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide.

In certain embodiments, the fusion polypeptide described herein has an amino acid sequence of a protein encoded by a nucleic acid molecule disclosed herein. Other useful proteins are substantially identical (e.g., at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, at least 99, at least 99.5% or greater) to one of these sequences and retain the functional activity of the protein of the corresponding full-length protein yet differ in amino acid sequence.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. Another, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877, incorporated herein by reference. Such an algorithm is incorporated into the NBLAST and XBLAST programs of Altschul, et al. (1990) J. Mol. Biol. 215:403-410, incorporated herein by reference. BLAST nucleotide searches can be performed with the NBLAST program, score=100, word length=12 to obtain nucleotide sequences homologous to a nucleic acid molecules featured in the invention. BLAST protein searches can be performed with the XBLAST program, score=50, word length=3 to obtain amino acid sequences homologous to protein molecules featured in the invention. To obtain gapped alignments for comparison purposes, Gapped BLAST can be utilized as described in Altschul et al. (1997) Nucleic Acids Res. 25:3389-3402, incorporated herein by reference. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See http://www.ncbi.nlm.nih.gov. Another non-limiting example of a mathematical algorithm utilized for the comparison of sequences is the algorithm of Myers and Miller, (1988) Comput Appl Biosci, 4:11-7, incorporated herein by reference. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448, incorporated herein by reference. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

An isolated fusion polypeptide (e.g., a fusion described herein), or a fragment thereof, can be used as an immunogen to generate antibodies using standard techniques for polyclonal and monoclonal antibody preparation. The full-length fusion polypeptide can be used or, alternatively, the invention provides antigenic peptide fragments for use as immunogens. The antigenic peptide of a protein featured in the invention comprises at least 8 (or at least 10, at least 15, at least 20, or at least 30 or more) amino acid residues of the amino acid sequence of one of the polypeptides featured in the invention, and encompasses an epitope of the protein such that an antibody raised against the peptide forms a specific immune complex with a marker featured in the invention to which the protein corresponds. Exemplary epitopes encompassed by the antigenic peptide are regions that are located on the surface of the protein, e.g., hydrophilic regions. Hydrophobicity sequence analysis, hydrophilicity sequence analysis, or similar analyses can be used to identify hydrophilic regions.

An immunogen typically is used to prepare antibodies by immunizing a suitable (i.e., immunocompetent) subject such as a rabbit, goat, mouse, or other mammal or vertebrate. An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

Accordingly, another aspect featured in the invention pertains to antibodies directed against a fusion polypeptide described herein. In one embodiment, the antibody molecule specifically binds to fusion molecule described herein, e.g., specifically binds to an epitope formed by the fusion. In embodiments the antibody can distinguish wild type genes that make up the fusion, from the fusion of the genes, e.g., the antibody can distinguish wild type genes, e.g., BRAF (or CEP89) from CEP89-BRAF.

The terms “antibody” and “antibody molecule” as used interchangeably herein refer to immunoglobulin molecules and immunologically active portions of immunoglobulin molecules, i.e., molecules that contain an antigen binding site which specifically binds an antigen, such as a polypeptide featured in the invention. A molecule which specifically binds to a given polypeptide featured in the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition,” as used herein, refers to a population of antibody molecules that contain only one species of an antigen binding site capable of immunoreaction with a particular epitope.

Polyclonal antibodies can be prepared as described above by immunizing a suitable subject with a fusion polypeptide as an immunogen. Antibody-producing cells can be obtained from the subject and used to prepare monoclonal antibodies by standard techniques, such as the hybridoma technique originally described by Kohler and Milstein (1975) Nature 256:495-497, the human B cell hybridoma technique (see Kozbor et al., 1983, Immunol. Today, 4:72), the EBV-hybridoma technique (see Cole et. al., pp. 77-96 In Monoclonal Antibodies and Cancer Therapy, Alan R. Liss, Inc., 1985) or trioma techniques, all incorporated herein by reference. The technology for producing hybridomas is well known (see generally Current Protocols in Immunology, Coligan et al. ed., John Wiley & Sons, New York, 1994, incorporated herein by reference). Hybridoma cells producing a monoclonal antibody are detected by screening the hybridoma culture supernatants for antibodies that bind the polypeptide of interest, e.g., using a standard ELISA assay.

Alternative to preparing monoclonal antibody-secreting hybridomas, a monoclonal antibody can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734, all incorporated herein by reference.

Additionally, recombinant antibodies, such as chimeric and humanized monoclonal antibodies, comprising both human and non-human portions can be made using standard recombinant DNA techniques. Such chimeric and humanized monoclonal antibodies can be produced by recombinant DNA techniques known in the art, for example using methods described in PCT Publication No. WO 87/02671; European Patent Application 184, 187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559; Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060, all incorporated herein by reference.

Completely human antibodies can be produced using transgenic mice which are incapable of expressing endogenous immunoglobulin heavy and light chains genes, but which can express human heavy and light chain genes. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

An antibody directed against a fusion polypeptide described herein (e.g., a monoclonal antibody) can be used to isolate the polypeptide by standard techniques, such as affinity chromatography or immunoprecipitation. Moreover, such an antibody can be used to detect the marker (e.g., in a cellular lysate or cell supernatant) in order to evaluate the level and pattern of expression of the marker. Detection can be facilitated by coupling the antibody to a detectable substance. Examples of detectable substances include, but are not limited to, various enzymes, prosthetic groups, fluorescent materials, luminescent materials, bioluminescent materials, and radioactive materials. Examples of suitable enzymes include, but are not limited to, horseradish peroxidase, alkaline phosphatase, β-galactosidase, or acetylcholinesterase; examples of suitable prosthetic group complexes include, but are not limited to, streptavidin/biotin and avidin/biotin; examples of suitable fluorescent materials include, but are not limited to, umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, but are not limited to, luciferase, luciferin, and aequorin, and examples of suitable radioactive materials include, but are not limited to, ¹²⁵I, ¹³¹I, ³⁵S or ³H.

An antibody directed against a fusion polypeptide described herein, can also be used diagnostically to monitor protein levels in tissues or body fluids (e.g., in a tumor cell-containing body fluid) as part of a clinical testing procedure, e.g., to, for example, determine the efficacy of a given treatment regimen.

### Antigens and Vaccines

Embodiments featured in the invention include preparations, e.g., antigenic preparations, of the entire fusion or a fragment thereof, e.g., a fragment capable of raising antibodies specific to the fusion protein, e.g., a fusion junction containing fragment (collectively referred to herein as a “fusion-specific polypeptides” or FSP). The preparation can include an adjuvant or other component.

An FSP can be used as an antigen or vaccine. For example, an FSP can be used as an antigen to immunize an animal, e.g., a rodent, e.g., a mouse or rat, rabbit, horse, goat, dog, or non-human primate, to obtain antibodies, e.g., fusion protein specific antibodies. In an embodiment a fusion specific antibody molecule is an antibody molecule described herein, e.g., a polyclonal. In other embodiments a fusion specific antibody molecule is monospecific, e.g., monoclonal, human, humanized, chimeric or other monospecific antibody molecule. An anti-fusion protein specific antibody molecule can be used to treat a subject having a cancer, e.g., a cancer described herein.

Embodiments featured include vaccine preparations that comprise an FSP capable of stimulating an immune response in a subject, e.g., by raising, in the subject, antibodies specific to the fusion protein. The vaccine preparation can include other components, e.g., an adjuvant. The vaccine preparations can be used to treat a subject having cancer, e.g., a cancer described herein.

### Rearrangement Based Cancer Vaccines

Embodiments featured in the invention include preparations of a fusion polypeptide described herein. The fusion polypeptide can be derived from, but is not limited to, any fusion molecule described herein.

A fusion junction polypeptide can be used as an antigen or vaccine, for the treatment of a disease. e.g., a cancer, e.g., a cancer described herein. For example, antigen presenting cells (APCs) derived from a patient with a disease, e.g., cancer, e.g., a cancer described herein; can be incubated with a fusion junction polypeptide, wherein the disease from which the patient's APCs are derived is known, has been determined, or is suspected of expressing the fusion molecule from which the fusion junction polypeptide is derived. In certain embodiments, the APCs are also incubated with one or more cytokines. In certain embodiments, the cytokine induces maturation of the APCs. In certain embodiments, the cytokine is one or more of GMCSF, TNF-alpha, IL-4, IL-2, IL-6, IL-7, IL-13, IL-15, HGF. In certain embodiments, the cytokine is GMCSF. The APCs are incubated with the fusion polypeptide under conditions which allow the APCs to uptake or endocytose the fusion polypeptide, and process the polypeptide for presentation on a cell surface molecule, e.g., major histocompatibility class MHC class I molecules. The cell culture conditions are known to one of skill in the art. The APCs can then be infused back into the same patient from whom the cells were derived.

In certain embodiments the APCs are purified prior to incubation with a fusion polypeptide. In certain embodiments, the APCs are dendritic cells. In certain embodiments, the APCs include one or more of dendritic cells, macrophages, and B cells. In certain embodiments, the APCs are incubated with one, two, three, four, or more fusion polypeptides.

In certain embodiments, the disclosure includes preparations of or a vaccine preparation of mature APCs which have been incubated with a fusion polypeptide described herein.

In certain embodiments, the method includes determining or acquiring a determination of whether a patient expresses a fusion molecule described herein. In certain embodiments, the method includes selecting a fusion polypeptide based on the determination of whether a patient expresses a fusion molecule described herein. In some embodiments, the method further comprises the incubation of APCs derived from the patient with the selected fusion polypeptide. In some embodiments, the method further comprises the infusion of the APCs back into the patient from which they were derived.

### Expression Vectors, Host Cells and Recombinant Cells

In another aspect, the invention includes vectors (e.g., expression vectors), containing a nucleic acid encoding a fusion polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a fusion nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors can be introduced into host cells to thereby produce a fusion polypeptide, including fusion proteins or polypeptides encoded by nucleic acids as described herein, mutant forms thereof, and the like).

The term “recombinant host cell” (or simply “host cell” or “recombinant cell”), as used herein, is intended to refer to a cell into which a recombinant expression vector has been introduced. It should be understood that such terms are intended to refer not only to the particular subject cell, but to the progeny of such a cell. Because certain modifications may occur in succeeding generations due to either mutation or environmental influences, such progeny may not, in fact, be identical to the parent cell, but are still included within the scope of the term “host cell” as used herein.

The recombinant expression vectors can be designed for expression of a fusion polypeptide (e.g., a fusion described herein) in prokaryotic or eukaryotic cells. For example, polypeptides featured in the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif., incorporated herein by reference. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B, and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion polypeptides described herein can be used in activity assays (e.g., direct assays or competitive assays described in detail below), or to generate antibodies specific for fusion polypeptides described herein.

To maximize recombinant protein expression in E. coli is to express the protein in a host bacteria with an impaired capacity to proteolytically cleave the recombinant protein (Gottesman, S., (1990) Gene Expression Technology: Methods in Enzymology 185, Academic Press, San Diego, Calif. 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences can be carried out by standard DNA synthesis techniques.

The fusion polypeptide expression vector can be a yeast expression vector, a vector for expression in insect cells, e.g., a baculovirus expression vector or a vector suitable for expression in mammalian cells.

When used in mammalian cells, the expression vector's control functions can be provided by viral regulatory elements. For example, commonly used promoters are derived from polyoma, Adenovirus 2, cytomegalovirus and Simian Virus 40.

In another embodiment, the promoter is an inducible promoter, e.g., a promoter regulated by a steroid hormone, by a polypeptide hormone (e.g., by means of a signal transduction pathway), or by a heterologous polypeptide (e.g., the tetracycline-inducible systems, “Tet-On” and “Tet-Off”; see, e.g., Clontech Inc., CA, Gossen and Bujard (1992) Proc. Natl. Acad. Sci. USA 89:5547, and Paillard (1989) Human Gene Therapy 9:983).

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the fetoprotein promoter (Campes and Tilghman (1989) Genes Dev. 3:537-546), all incorporated herein by reference.

The invention further provides a recombinant expression vector comprising a DNA molecule featured in the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus.

Another aspect the invention provides a host cell which includes a nucleic acid molecule described herein, e.g., a fusion nucleic acid molecule described herein within a recombinant expression vector or a fusion nucleic acid molecule described herein containing sequences which allow it to homologous recombination into a specific site of the host cell's genome.

A host cell can be any prokaryotic or eukaryotic cell. For example, a fusion polypeptide can be expressed in bacterial cells (such as E. coli), insect cells, yeast or mammalian cells (such as Chinese hamster ovary cells (CHO) or COS cells, e.g., COS-7 cells, CV-1 origin SV40 cells; Gluzman (1981) Cell 23:175-182). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell can be used to produce (e.g., express) a fusion polypeptide (e.g., a fusion molecule described herein). Accordingly, the invention further provides methods for producing a fusion polypeptide using the host cells. In one embodiment, the method includes culturing the host cell (into which a recombinant expression vector encoding a polypeptide has been introduced) in a suitable medium such that the fusion polypeptide is produced. In another embodiment, the method further includes isolating a fusion polypeptide from the medium or the host cell.

In another aspect, the invention features, a cell or purified preparation of cells which include a fusion molecule described herein transgene, or which otherwise misexpress the fusion. For example, a cell or purified preparation of cells which include a FGFR3-TACC3 fusion transgene, or which otherwise misexpress FGFR3-TACC3 fusion.

The cell preparation can consist of human or non-human cells, e.g., rodent cells, e.g., mouse or rat cells, rabbit cells, or pig cells. In embodiments, the cell or cells include a fusion transgene, e.g., a heterologous form of a fusion described herein, e.g., a gene derived from humans (in the case of a non-human cell) or a fusion transgene, e.g., a heterologous form of a fusion described herein. The fusion transgene can be misexpressed, e.g., overexpressed or under expressed. In other preferred embodiments, the cell or cells include a gene that mis-expresses an endogenous fusion, e.g., a gene the expression of which is disrupted, e.g., a knockout. Such cells can serve as a model for studying disorders that are related to mutated or misexpressed fusion alleles (e.g., cancers) or for use in drug screening, as described herein.

### Therapeutic Methods

Alternatively, or in combination with the methods described herein, the invention features a method of treating a neoplasm, a cancer or a tumor harboring a fusion moelcule described herein. The methods include administering an anti-cancer agent, e.g., a kinase inhibitor, alone or in combination, e.g., in combination with other chemotherapeutic agents or procedures, in an amount sufficient to reduce or inhibit the tumor cell growth, and/or treat or prevent the cancer(s), in the subject.

“Treat,” “treatment,” and other forms of this word refer to the administration of a kinase inhibitor, alone or in combination with a second agent to impede growth of a cancer, to cause a cancer to shrink by weight or volume, to extend the expected survival time of the subject and or time to progression of the tumor or the like. In those subjects, treatment can include, but is not limited to, inhibiting tumor growth, reducing tumor mass, reducing size or number of metastatic lesions, inhibiting the development of new metastatic lesions, prolonged survival, prolonged progression-free survival, prolonged time to progression, and/or enhanced quality of life.

As used herein, unless otherwise specified, the terms “prevent,” “preventing” and “prevention” contemplate an action that occurs before a subject begins to suffer from the re-growth of the cancer and/or which inhibits or reduces the severity of the cancer.

As used herein, and unless otherwise specified, a “therapeutically effective amount” of a compound is an amount sufficient to provide a therapeutic benefit in the treatment or management of the cancer, or to delay or minimize one or more symptoms associated with the cancer. A therapeutically effective amount of a compound means an amount of therapeutic agent, alone or in combination with other therapeutic agents, which provides a therapeutic benefit in the treatment or management of the cancer. The term “therapeutically effective amount” can encompass an amount that improves overall therapy, reduces or avoids symptoms or causes of the cancer, or enhances the therapeutic efficacy of another therapeutic agent.

As used herein, and unless otherwise specified, a “prophylactically effective amount” of a compound is an amount sufficient to prevent re-growth of the cancer, or one or more symptoms associated with the cancer, or prevent its recurrence. A prophylactically effective amount of a compound means an amount of the compound, alone or in combination with other therapeutic agents, which provides a prophylactic benefit in the prevention of the cancer. The term “prophylactically effective amount” can encompass an amount that improves overall prophylaxis or enhances the prophylactic efficacy of another prophylactic agent.

As used herein, the term “patient” or “subject” refers to an animal, typically a human (i.e., a male or female of any age group, e.g., a pediatric patient (e.g., infant, child, adolescent) or adult patient (e.g., young adult, middle-aged adult or senior adult) or other mammal, such as a primate (e.g., cynomolgus monkey, rhesus monkey); commercially relevant mammals such as cattle, pigs, horses, sheep, goats, cats, and/or dogs; and/or birds, including commercially relevant birds such as chickens, ducks, geese, and/or turkeys, that will be or has been the object of treatment, observation, and/or experiment. When the term is used in conjunction with administration of a compound or drug, then the patient has been the object of treatment, observation, and/or administration of the compound or drug.

In certain embodiments, the cancer includes, but is not limited to, a solid tumor, a soft tissue tumor, and a metastatic lesion (e.g., a cancer as described herein). In one embodiment, the cancer is chosen from lung adenocarcinoma, cervical adenocarcinoma, uterus endometrial adenocarcinoma, glioblastoma, melanoma (such as spitzoid or Spitz melanoma), spindle cell sarcoma, ameloblastic fibrosarcoma, adenocarcinoma, cholangiocarcinoma, urothelial (transitional cell) carcinoma, ovarian epithelial carcinoma, colorectal adenocarcinoma, breast carcinoma, prostate carcinoma, or pancreas ductal adenocarcinoma. In one embodiment, the cancer is chosen from a lung cancer, a pancreatic cancer, melanoma, a colorectal cancer, an esophageal-gastric cancer, a thyroid cancer, or an adenocarcinoma. In other embodiment, the lung cancer is chosen from one or more of the following: non-small cell lung cancer (NSCLC), small cell lung cancer (SCLC), squamous cell carcinoma (SCC), adenocarcinoma of the lung, bronchogenic carcinoma, or a combination thereof. In one embodiment, the lung cancer is NSCLC or SCC.

In other embodiments, the cancer is chosen from lung cancer, thyroid cancer, colorectal cancer, adenocarcinoma, melanoma, B cell cancer, breast cancer, bronchus cancer, cancer of the oral cavity or pharynx, cancer of hematological tissues, cervical cancer, colon cancer, esophageal cancer, esophageal-gastric cancer, gastric cancer, kidney cancer, liver cancer, multiple myeloma, ovarian cancer, pancreatic cancer, prostate cancer, salivary gland cancer, small bowel or appendix cancer, stomach cancer, testicular cancer, urinary bladder cancer, uterine or endometrial cancer, inflammatory myofibroblastic tumors, gastrointestinal stromal tumor (GIST), and the like.

In certain embodiments, the neoplasm or neoplastic cell is a benign, pre-malignant, malignant (cancer) or metastasis.

### Kinase Inhibitors

In one embodiment, the anti-cancer agent is a kinase inhibitor. For example, the kinase inhibitor is a multi-kinase inhibitor or a specific inhibitor. Exemplary kinase inhibitors include, but are not limited to, alisertib (MLN8237), axitinib (AG013736), bosutinib (SKI-606), cediranib (RECENTIN™, AZD2171), crizotinib (PF-02341066, Xalkori), dasatinib (SPRYCEL®, BMS-354825), deforolimus (AP23573/MK-8669), dovitinib lactate (TK1258, CHIR-258), enzastaurin (LY317615), everolimus (RAD001), erlotinib (TARCEVA®), fostamatinib (FosD/R788), gefitinib (IRESSA®), imatinib (Gleevec®, CGP57148B, STI-571), ibrutinib (PCI-32765), lapatinib (TYKERB®, TYVERB®), lestaurtinib (CEP-701), neratinib (HKI-272), nilotinib (TASIGNA®), pacritinib (SB1518), ponatinib (Iclusig), semaxanib (semaxinib, SU5416), sorafenib (NEXAVAR®), sunitinib (SUTENT®, SU11248), temsirolimus (CCI-779/Torisel), tipifarnib (Zamestra, R115777), tivozanib (AV-951), toceranib (PALLADIA®), vandetanib, vatalanib (PTK787, PTK/ZK), ENMD-2076, PCI-32765, AC220, BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, OSI-930, MM-121, XL-184, XL-647, LDK378, GS-1101 (CAL-101), MK-2206, perifosine, LGX818, BMS-908662, PLX3603, RAF265, RO5185426, trametinib, cabozantinib, AZ64, AP26113, X-276, X-376, X-396, CH5424802 (AF-802), GSK1838705, ASP3026, PHA-E429, CRL151104A and XL228. Table 10 describes some kinase inhibitors against BRAF, BRAF, NTRK1, ALK and ROS1.

For these inhibitors, a pharmaceutically effective dose amount is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients (i.e., kinase inhibitors) is administered.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient. It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

For example, the dosing of the antitumor efficacy of crizotinib was initially demonstrated to be 250 mg, orally, given twice a day, based on results from the initial phase I dose escalation studies for a variety of cancers (Sahu, A. et al., South Asian J Cancer. 2013 2(2): 91-97). Another kinase inhibitor, vemurafenib has a maximum tolerated dose of 60 mg twice daily as established by a phase I trial and is shown to be effective against melanoma patients at this dose (Chapman, P. et al, N Engl J Med. 2011; 364(26): 2507-2516).

TABLE 10
Kinase inhibitors against certain tyrosine
and serine/threonine protein kinases

| Kinase | Inhibitors |
|---|---|
| BRAF | vemurafenib (also known as RG7204; or PLX4032; or Zelboraf); |
| | GDC-0879; PLX-4702; AZ628; dabrafenib (GSK2118346); |
| | LGX818; BMS-908662, PLX3603, RAF265, RO5185426, |
| | trametinib; or Sorafenib Tosylate |
| RET | pyrazolo-pyrimidines, e.g., PP1 and PP2; indocarbazole |
| | derivatives, e.g., CEP-701 and CEP-751; 2-indolinone, e.g., |
| | RPI-1; and quinazoline, e.g., ZD6474; or TG101209; |
| | vandetanib, cabozantinib |
| NTRK1 | lestaurtinib (CEP-701); AZ-23; indenopyrrolocarboazole |
| | 12a; oxindole 3; isothiazole 5n thiazole 20h; |
| | dasatinib; AZ64 |
| ALK | TAE-684 (also referred to herein as “NVP-TAE694”), |
| | PF02341066 (also referred to herein as “crizotinib” or |
| | “1066”), AF-802, LDK-378, ASP-3026, CEP-37440, |
| | CEP-28122, CEP-108050, MK-2206, perifosine, sorafertib and |
| | AP26113. Additional examples of AL kinase inhibitors are |
| | described in examples 3-39 of WO 2005016894 by Garcia- |
| | Echeverria C, et al. |
| ROS1 | Ganetespib; Crizotinib; TAE684; AP26113, X-276, X-376, |
| | X-396, CH5424802 (AF-802), GSK1838705, ASP3026, |
| | PHA-E429, CRL151104A Additional examples of kinase |
| | inhibitors are described in de la Bellacasa R. P. et al, |
| | Transl Lung Cancer Res 2013; 2(2): 72-86. |

In other embodiments, the anti-cancer agent is a fusion antagonist inhibits the expression of nucleic acid encoding a fusion described herein. Examples of such fusion antagonists include nucleic acid molecules, for example, antisense molecules, ribozymes, RNAi, triple helix molecules that hybridize to a nucleic acid encoding a fusion described herein, or a transcription regulatory region, and blocks or reduces mRNA expression of a fusion described herein.

In other embodiments, the kinase inhibitor is administered in combination with a second therapeutic agent or a different therapeutic modality, e.g., anti-cancer agents, and/or in combination with surgical and/or radiation procedures.

By “in combination with,” it is not intended to imply that the therapy or the therapeutic agents must be administered at the same time and/or formulated for delivery together, although these methods of delivery are within the scope of the invention. The pharmaceutical compositions can be administered concurrently with, prior to, or subsequent to, one or more other additional therapies or therapeutic agents. In general, each agent will be administered at a dose and/or on a time schedule determined for that agent. In will further be appreciated that the additional therapeutic agent utilized in this combination can be administered together in a single composition or administered separately in different compositions. The particular combination to employ in a regimen will take into account compatibility of the inventive pharmaceutical composition with the additional therapeutically active agent and/or the desired therapeutic effect to be achieved.

For example, the second therapeutic agent can be a cytotoxic or a cytostatic agent. Exemplary cytotoxic agents include antimicrotubule agents, topoisomerase inhibitors, or taxanes, antimetabolites, mitotic inhibitors, alkylating agents, intercalating agents, agents capable of interfering with a signal transduction pathway, agents that promote apoptosis and radiation. In yet other embodiments, the methods can be used in combination with immunomodulatory agents, e.g., IL-1, 2, 4, 6, or 12, or interferon alpha or gamma, or immune cell growth factors such as GM-CSF.

Anti-cancer agents, e.g., kinase inhibitors, used in therapeutic methods can be evaluated using the screening assays described herein. In one embodiment, the anti-cancer agents are evaluated in a cell-free system, e.g., a cell lysate or in a reconstituted system. In other embodiments, the anti-cancer agents are evaluated in a cell in culture, e.g., a cell expressing fusion molecule described herein (e.g., a mammalian cell, a tumor cell or cell line, a recombinant cell). In yet other embodiments, the anti-cancer agents are evaluated cell in vivo (a fusion molecule-expressing cell present in a subject, e.g., an animal subject (e.g., an in vivo animal model).

Exemplary parameters evaluated include one or more of:

(i) a change in binding activity, e.g., direct binding of the candidate agent to a fusion polypeptide described herein; a binding competition between a known ligand and the candidate agent to a fusion polypeptide described herein;

(ii) a change in kinase activity, e.g., phosphorylation levels of a fusion polypeptide described herein (e.g., an increased or decreased autophosphorylation); or a change in phosphorylation of a target of an kinase;

(iii) a change in an activity of a cell containing a fusion described herein (e.g., a tumor cell or a recombinant cell), e.g., a change in proliferation, morphology or tumorigenicity of the cell;

(iv) a change in tumor present in an animal subject, e.g., size, appearance, proliferation, of the tumor; or

(v) a change in the level, e.g., expression level, of a fusion polypeptide described herein or nucleic acid molecule described herein.

In one embodiment, a change in a cell free assay in the presence of a candidate agent is evaluated. For example, an activity of a fusion molecule described herein, or interaction of a fusion molecule described herein with a downstream ligand can be detected.

In other embodiments, a change in an activity of a cell is detected in a cell in culture, e.g., a cell expressing a fusion molecule described herein (e.g., a mammalian cell, a tumor cell or cell line, a recombinant cell). In one embodiment, the cell is a recombinant cell that is modified to express a fusion nucleic acid described herein, e.g., is a recombinant cell transfected with a fusion nucleic acid described herein. The transfected cell can show a change in response to the expressed fusion molecule described herein, e.g., increased proliferation, changes in morphology, increased tumorigenicity, and/or acquired a transformed phenotype. A change in any of the activities of the cell, e.g., the recombinant cell, in the presence of the candidate agent can be detected. For example, a decrease in one or more of: proliferation, tumorigenicity, transformed morphology, in the presence of the candidate agent can be indicative of an inhibitor of a fusion molecule described herein. In other embodiments, a change in binding activity or phosphorylation as described herein is detected.

In yet other embodiment a change in a tumor present in an animal subject (e.g., an in vivo animal model) is detected. In one embodiment, the animal model is a tumor containing animal or a xenograft comprising cells expressing a fusion molecule described herein (e.g., tumorigenic cells expressing a fusion molecule described herein). The anti-cancer agents can be administered to the animal subject and a change in the tumor is detected. In one embodiment, the change in the tumor includes one or more of a tumor growth, tumor size, tumor burden, survival, is evaluated. A decrease in one or more of tumor growth, tumor size, tumor burden, or an increased survival is indicative that the candidate agent is an inhibitor.

Therapeutic compositions may include a pharmaceutically effective amount of the desired compounds in a pharmaceutically acceptable carrier or diluent. Acceptable carriers or diluents for therapeutic use are well known in the pharmaceutical art, and are described, for example, in REMINGTON'S PHARMACEUTICAL SCIENCES, Mack Publishing Co. (A. R. Gennaro, Ed. 1985). For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient. It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

An inhibiting therapeutic against ROS1, ALK, BRAF, RET, or NTRK1 useful in the practice of the invention may comprise a single compound as described above, or a combination of multiple compounds, whether in the same class of inhibitor (i.e. antibody inhibitor), or in different classes (i.e., antibody inhibitors and small-molecule inhibitors). Such combination of compounds may increase the overall therapeutic effect in inhibiting the progression of a fusion protein-expressing cancer. For example, the therapeutic composition may a small molecule inhibitor, or in combination with other inhibitors targeting ROS1, ALK, BRAF, RET, or NTRK1 activity and/or other small molecule inhibitors. The therapeutic composition may also comprise one or more non-specific chemotherapeutic agent in addition to one or more targeted inhibitor. Such combinations have recently been shown to provide a synergistic tumor killing effect in many cancers. The screening methods and assays are described in more detail herein below.

### Screening Methods

In another aspect, the invention features a method, or assay, for screening for agents that modulate, e.g., inhibit, the expression or activity of a fusion molecule described herein. The method includes contacting a fusion molecule described herein, or a cell expressing a fusion molecule described herein, with a candidate agent; and detecting a change in a parameter associated with a fusion molecule described herein, e.g., a change in the expression or an activity of the fusion molecule described herein. The method can, optionally, include comparing the treated parameter to a reference value, e.g., a control sample (e.g., comparing a parameter obtained from a sample with the candidate agent to a parameter obtained from a sample without the candidate agent). In one embodiment, if a decrease in expression or activity of the fusion molecule described herein is detected, the candidate agent is identified as an inhibitor. In another embodiment, if an increase in expression or activity of the fusion molecule described herein is detected, the candidate agent is identified as an activator. In certain embodiments, the fusion molecule described herein is a nucleic acid molecule or a polypeptide as described herein.

In one embodiment, the contacting step is effected in a cell-free system, e.g., a cell lysate or in a reconstituted system. In other embodiments, the contacting step is effected in a cell in culture, e.g., a cell expressing a fusion molecule described herein (e.g., a mammalian cell, a tumor cell or cell line, a recombinant cell). In yet other embodiments, the contacting step is effected in a cell in vivo (a fusion molecule described herein-expressing cell present in a subject, e.g., an animal subject (e.g., an in vivo animal model).

Exemplary parameters evaluated include one or more of:

(i) a change in binding activity, e.g., direct binding of the candidate agent to a fusion polypeptide described herein; a binding competition between a known ligand and the candidate agent to a fusion polypeptide described herein;

(ii) a change in kinase activity, e.g., phosphorylation levels of a fusion polypeptide described herein (e.g., an increased or decreased autophosphorylation); or a change in phosphorylation of a target of an kinase. In certain embodiments, a change in kinase activity, e.g., phosphorylation, is detected by any of Western blot (e.g., using an anti-BRAF or anti-CEP89 antibody; a phosphor-specific antibody, detecting a shift in the molecular weight of a CEP89-BRAF fusion polypeptide), mass spectrometry, immunoprecipitation, immunohistochemistry, immunomagnetic beads, among others;

(iii) a change in an activity of a cell containing a fusion molecule described herein (e.g., a tumor cell or a recombinant cell), e.g., a change in proliferation, morphology or tumorigenicity of the cell;

(iv) a change in tumor present in an animal subject, e.g., size, appearance, proliferation, of the tumor; or

(v) a change in the level, e.g., expression level, of a fusion polypeptide described herein or nucleic acid molecule described herein.

In one embodiment, a change in a cell free assay in the presence of a candidate agent is evaluated. For example, an activity of a fusion molecule described herein, or interaction of a fusion molecule described herein with a downstream ligand can be detected. In one embodiment, a fusion polypeptide described herein is contacted with a ligand, e.g., in solution, and a candidate agent is monitored for an ability to modulate, e.g., inhibit, an interaction, e.g., binding, between the fusion polypeptide described herein and the ligand. In one exemplary assay, purified fusion protein described herein is contacted with a ligand, e.g., in solution, and a candidate agent is monitored for an ability to inhibit interaction of the fusion protein with the ligand, or to inhibit phosphorylation of the ligand by the fusion protein. An effect on an interaction between the fusion protein and a ligand can be monitored by methods known in the art, such as by absorbance, and an effect on phosphorylation of the ligand can be assayed. e.g., by Western blot, immunoprecipitation, or immunomagnetic beads.

In other embodiments, a change in an activity of a cell is detected in a cell in culture, e.g., a cell expressing a fusion molecule described herein (e.g., a mammalian cell, a tumor cell or cell line, a recombinant cell). In one embodiment, the cell is a recombinant cell that is modified to express a fusion nucleic acid described herein, e.g., is a recombinant cell transfected with a fusion nucleic acid described herein. The transfected cell can show a change in response to the expressed fusion molecule, e.g., increased proliferation, changes in morphology, increased tumorigenicity, and/or acquired a transformed phenotype. A change in any of the activities of the cell, e.g., the recombinant cell, in the presence of the candidate agent can be detected. For example, a decrease in one or more of: proliferation, tumorigenicity, transformed morphology, in the presence of the candidate agent can be indicative of an inhibitor of a fusion molecule described herein. In other embodiments, a change in binding activity or phosphorylation as described herein is detected.

In an exemplary cell-based assay, a nucleic acid comprising a fusion molecule described herein can be expressed in a cell, such as a cell (e.g., a mammalian cell) in culture. The cell containing a nucleic acid expressing the fusion molecule can be contacted with a candidate agent, and the cell is monitored for an effect of the candidate agent. A candidate agent that causes decreased cell proliferation or cell death can be determined to be a candidate for treating a tumor (e.g., a cancer) that carries a fusion described herein.

In one embodiment, a cell containing a nucleic acid expressing a fusion molecule described herein can be monitored for expression of the fusion protein. Protein expression can be monitored by methods known in the art, such as by, e.g., mass spectrometry (e.g., tandem mass spectrometry), a reporter assay (e.g., a fluorescence-based assay), Western blot, and immunohistochemistry. By one method, decreased fusion expression is detected. A candidate agent that causes decreased expression of the fusion protein as compared to a cell that does not contain the nucleic acid fusion can be determined to be a candidate for treating a tumor (e.g., a cancer) that carries a fusion described herein.

A cell containing a nucleic acid expressing a fusion molecule described herein can be monitored for altered kinase activity. Kinase activity can be assayed by measuring the effect of a candidate agent on a known kinase target protein.

In yet other embodiment a change in a tumor present in an animal subject (e.g., an in vivo animal model) is detected. In one embodiment, the animal model is a tumor containing animal or a xenograft comprising cells expressing a fusion molecule described herein (e.g., tumorigenic cells expressing a fusion molecule described herein). The candidate agent can be administered to the animal subject and a change in the tumor is detected. In one embodiment, the change in the tumor includes one or more of a tumor growth, tumor size, tumor burden, survival, is evaluated. A decrease in one or more of tumor growth, tumor size, tumor burden, or an increased survival is indicative that the candidate agent is an inhibitor.

In one exemplary animal model, a xenograft is created by injecting cells into mouse. A candidate agent is administered to the mouse, e.g., by injection (such as subcutaneous, intraperitoneal, or tail vein injection, or by injection directly into the tumor) or oral delivery, and the tumor is observed to determine an effect of the candidate anti-cancer agent. The health of the animal is also monitored, such as to determine if an animal treated with a candidate agent survives longer. A candidate agent that causes growth of the tumor to slow or stop, or causes the tumor to shrink in size, or causes decreased tumor burden, or increases survival time, can be considered to be a candidate for treating a tumor (e.g., a cancer) that carries a fusion described herein.

In another exemplary animal assay, cells expressing a fusion described herein are injected into the tail vein. e.g., of a mouse, to induce metastasis. A candidate agent is administered to the mouse, e.g., by injection (such as subcutaneous, intraperitoneal, or tail vein injection, or by injection directly into the tumor) or oral delivery, and the tumor is observed to determine an effect of the candidate anti-cancer agent. A candidate agent that inhibits or prevents or reduces metastasis, or increases survival time, can be considered to be a candidate for treating a tumor (e.g., a cancer) that carries a fusion described herein.

Cell proliferation can be measured by methods known in the art, such as PCNA (Proliferating cell nuclear antigen) assay, 5-bromodeoxyuridine (BrdUrd) incorporation, Ki-67 assay, mitochondrial respiration, or propidium iodide staining. Cells can also be measured for apoptosis, such as by use of a TUNEL (Terminal Deoxynucleotide Transferase dUTP Nick End Labeling) assay. Cells can also be assayed for presence of angiogenesis using methods known in the art, such as by measuring endothelial tube formation or by measuring the growth of blood vessels from subcutaneous tissue, such as into a solid gel of basement membrane.

In other embodiments, a change in expression of a fusion molecule described herein can be monitored by detecting the nucleic acid or protein levels, e.g., using the methods described herein.

In certain embodiments, the screening methods described herein can be repeated and/or combined. In one embodiment, a candidate agent that is evaluated in a cell-free or cell-based described herein can be further tested in an animal subject.

In one embodiment, the candidate agent is identified and re-tested in the same or a different assay. For example, a test compound is identified in an in vitro or cell-free system, and re-tested in an animal model or a cell-based assay. Any order or combination of assays can be used. For example, a high throughput assay can be used in combination with an animal model or tissue culture.

Candidate agents suitable for use in the screening assays described herein include, e.g., small molecule compounds, nucleic acids (e.g., siRNA, aptamers, short hairpin RNAs, antisense oligonucleotides, ribozymes, antagomirs, microRNA mimics or DNA, e.g., for gene therapy) or polypeptides, e.g., antibodies (e.g., full length antibodies or antigen-binding fragments thereof, Fab fragments, or scFv fragments). The candidate anti-cancer agents can be obtained from a library (e.g., a commercial library), or can be rationally designed, such as to target an active site in a functional domain (e.g., a kinase domain).

In other embodiments, the method, or assay, includes providing a step based on proximity-dependent signal generation, e.g., a two-hybrid assay that includes a first fusion protein (e.g., a fusion protein described herein), and a second fusion protein (e.g., a ligand), contacting the two-hybrid assay with a test compound, under conditions wherein said two hybrid assay detects a change in the formation and/or stability of the complex, e.g., the formation of the complex initiates transcription activation of a reporter gene.

In one non-limiting example, the three-dimensional structure of the active site of fusion molecule described herein is determined by crystallizing the complex formed by the fusion molecule and a known inhibitor. Rational drug design is then used to identify new test agents by making alterations in the structure of a known inhibitor or by designing small molecule compounds that bind to the active site of the fusion.

The candidate agents can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann. R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad. Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

The interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means known in the art (e.g., using a fluorimeter).

In another embodiment, determining the ability of the fusion protein to bind to a target molecule can be accomplished using real-time Biomolecular Interaction Analysis (BIA) (see, e.g., Sjolander, S. and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

### Nucleic Acid Inhibitors

In another embodiment, a fusion inhibitor inhibits the expression of a nucleic acid encoding a fusion described herein. Examples of such fusion inhibitors include nucleic acid molecules, for example, antisense molecules, dsRNA, siRNA, ribozymes, or triple helix molecules, which hybridize to a nucleic acid encoding a fusion described herein, or a transcription regulatory region, and blocks or reduces mRNA expression of the fusion. Accordingly, isolated nucleic acid molecules that are nucleic acid inhibitors, e.g., antisense, siRNA, RNAi, to a fusion-encoding nucleic acid molecule are provided.

### Antisense

In some embodiments, the nucleic acid fusion inhibitor is an antisense nucleic acid molecule. An “antisense” nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire fusion coding strand, or to only a portion thereof. In another embodiment, the antisense nucleic acid molecule is antisense to a “noncoding region” of the coding strand of a nucleotide sequence encoding fusion (e.g., the 5′ and 3′ untranslated regions). Anti-sense agents can include, for example, from about 8 to about 80 nucleobases (i.e., from about 8 to about 80 nucleotides), e.g., about 8 to about 50 nucleobases, or about 12 to about 30 nucleobases. Antisense compounds include ribozymes, external guide sequence (EGS) oligonucleotides (oligozymes), and other short catalytic RNAs or catalytic oligonucleotides which hybridize to the target nucleic acid and modulate its expression. Antisense compounds can include a stretch of at least eight consecutive nucleobases that are complementary to a sequence in the target gene. An oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

Hybridization of antisense oligonucleotides with mRNA can interfere with one or more of the normal functions of mRNA. The functions of mRNA to be interfered with include all key functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA.

Exemplary antisense compounds include DNA or RNA sequences that specifically hybridize to the target nucleic acid, e.g., the mRNA encoding a fusion described herein. The complementary region can extend for between about 8 to about 80 nucleobases. The compounds can include one or more modified nucleobases. Modified nucleobases may include, e.g., 5-substituted pyrimidines such as 5-iodouracil, 5-iodocytosine, and C5-propynyl pyrimidines such as C5-propynylcytosine and C5-propynyluracil. Other suitable modified nucleobases include N⁴—(C₁-C₁₂) alkylaminocytosines and N⁴,N⁴—(C₁-C₁₂) dialkylaminocytosines. Modified nucleobases may also include 7-substituted-8-aza-7-deazapurines and 7-substituted-7-deazapurines such as, for example, 7-iodo-7-deazapurines, 7-cyano-7-deazapurines, 7-aminocarbonyl-7-deazapurines. Examples of these include 6-amino-7-iodo-7-deazapurines, 6-amino-7-cyano-7-deazapurines, 6-amino-7-aminocarbonyl-7-deazapurines, 2-amino-6-hydroxy-7-iodo-7-deazapurines, 2-amino-6-hydroxy-7-cyano-7-deazapurines, and 2-amino-6-hydroxy-7-aminocarbonyl-7-deazapurines. Furthermore, N⁶—(C₁-C₁₂) alkylaminopurines and N⁶,N⁶—(C₁-C₁₂) dialkylaminopurines, including N⁶-methylaminoadenine and N⁶,N⁶-dimethylaminoadenine, are also suitable modified nucleobases. Similarly, other 6-substituted purines including, for example, 6-thioguanine may constitute appropriate modified nucleobases. Other suitable nucleobases include 2-thiouracil, 8-bromoadenine, 8-bromoguanine, 2-fluoroadenine, and 2-fluoroguanine. Derivatives of any of the aforementioned modified nucleobases are also appropriate. Substituents of any of the preceding compounds may include C₁-C₃₀ alkyl, C₂-C₃a alkenyl, C₂-C₃₀ alkynyl, aryl, aralkyl, heteroaryl, halo, amino, amido, nitro, thio, sulfonyl, carboxyl, alkoxy, alkylcarbonyl, alkoxycarbonyl, and the like. Descriptions of other types of nucleic acid agents are also available. See, e.g., U.S. Pat. Nos. 4,987,071; 5,116,742; and 5,093,246; Woolf et al. (1992) Proc Natl Acad. Sci USA; Antisense RNA and DNA, D. A. Melton, Ed., Cold Spring Harbor Laboratory, Cold Spring Harbor, N.Y. (1988); 89:7305-9; Haselhoff and Gerlach (1988) Nature 334:585-59; Helene. C. (1991) Anticancer Drug Des. 6:569-84; Helene (1992) Ann. N. Y Acad. Sci. 660:27-36; and Maher (1992) Bioassays 14:807-15.

In yet another embodiment, the antisense nucleic acid molecule is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The antisense nucleic acid molecules are typically administered to a subject (e.g., by direct injection at a tissue site), or generated in situ such that they hybridize with or bind to cellular mRNA and/or genomic DNA encoding a fusion described herein to thereby inhibit expression of the protein, e.g., by inhibiting transcription and/or translation. Alternatively, antisense nucleic acid molecules can be modified to target selected cells and then be administered systemically. For systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

### Ribozymes

In another embodiment, an antisense nucleic acid featured in the invention is a ribozyme. A ribozyme having specificity for a fusion-encoding nucleic acid can include one or more sequences complementary to the nucleotide sequence of a fusion cDNA disclosed herein, and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a fusion-encoding mRNA. See, e.g., Cech et al. U.S. Pat. No. 4,987,071; and Cech et al. U.S. Pat. No. 5,116,742. Alternatively, fusion mRNA can be used to select a catalytic RNA having a specific ribonuclease activity from a pool of RNA molecules. See, e.g., Bartel, D, and Szostak, J. W. (1993) Science 261:1411-1418.

### Triple Helix Molecules

Inhibition of a fusion gene described herein can be accomplished by targeting nucleotide sequences complementary to the regulatory region of the fusion to form triple helical structures that prevent transcription of the fusion gene in target cells. See generally, Helene, C. (1991) Anticancer Drug Des. 6:569-84; Helene, C. i (1992) Ann. N.Y. Acad. Sci. 660:27-36; and Maher, L. J. (1992) Bioassays 14:807-15. The potential sequences that can be targeted for triple helix formation can be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

dsRNAs

In some embodiments, the nucleic acid fusion inhibitor is a dsRNA molecule, dsRNAs having a duplex structure of between about 20 and 23 base pairs, e.g., 21, base pairs are effective at inducing RNA interference (RNAi) (Elbashir et al., EMBO 2001, 20:6877-6888). However, others have found that shorter or longer RNA duplex structures can also be effective (Chu and Rana (2007) RNA 14:1714-1719; Kim et al. (2005) Nat Biotech 23:222-226).

In one embodiment, the dsRNA, is un-modified, and does not comprise, e.g., chemical modifications and/or conjugations known in the art or described herein. In another embodiment, the dsRNA, is chemically modified to enhance stability or other beneficial characteristics. The dsRNA can be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. While a target sequence of a dsRNA can be generally about 15-30 nucleotides in length, there is wide variation in the suitability of particular sequences in this range for directing cleavage of any given target RNA. Various software packages and the guidelines set out herein provide guidance for the identification of optimal target sequences for any given gene target, but an empirical approach can also be taken in which a “window” or “mask” of a given size (as a non-limiting example, 21 nucleotides) is literally or figuratively (including, e.g., in silico) placed on the target RNA sequence to identify sequences in the size range that can serve as target sequences. By moving the sequence “window” progressively one nucleotide upstream or downstream of an initial target sequence location, the next potential target sequence can be identified, until the complete set of possible sequences is identified for any given target size selected. This process, coupled with systematic synthesis and testing of the identified sequences (using assays as described herein or as known in the art) to identify those sequences that perform optimally can identify those RNA sequences that, when targeted with a dsRNA molecule, mediate the best inhibition of target gene expression. Thus, while the sequences identified herein represent effective target sequences, it is contemplated that further optimization of inhibition efficiency can be achieved by progressively “walking the window” one nucleotide upstream or downstream of the given sequences to identify sequences with equal or better inhibition characteristics.

In some embodiments, the nucleic acid fusion inhibitor is a siRNA molecule. siRNAs are small double stranded RNAs (dsRNAs) that optionally include overhangs. For example, the duplex region of an siRNA is about 18 to 25 nucleotides in length, e.g., about 19, 20, 21, 22, 23, or 24 nucleotides in length. Typically, the siRNA sequences are exactly complementary to the target mRNA. dsRNAs and siRNAs in particular can be used to silence gene expression in mammalian cells (e.g., human cells). siRNAs also include short hairpin RNAs (shRNAs) with 29-base-pair stems and 2-nucleotide 3′ overhangs. See, e.g., Clemens et al. (2000) Proc. Natl. Acad. Sci. USA 97:6499-6503; Billy et al. (2001) Proc. Natl. Sci. USA 98:14428-14433; Elbashir et al. (2001) Nature, 411:494-8; Yang et al. (2002) Proc. Natl. Acad. Sci. USA 99:9942-9947; Siolas et al. (2005), Nat. Biotechnol. 23(2):227-31; 20040086884; U.S. 20030166282; 20030143204; 20040038278; and 20030224432.

### Modifications of Nucleic Acid Fusion Inhibitor Molecules

A nucleic acid fusion inhibitor can be modified to enhance or obtain beneficial characteristics. For example, a nucleic acid fusion inhibitor can be modified at the base moiety, sugar moiety or phosphate backbone to improve, e.g., the stability, hybridization, or solubility of the molecule. For non-limiting examples of synthetic oligonucleotides with modifications see Toulmé (2001) Nature Biotech. 19:17 and Faria et al. (2001) Nature Biotech. 19:40-44. Such phosphoramidite oligonucleotides can be effective antisense agents.

A nucleic acid fusion inhibitor molecule can be modified to include one or more bridged nucleic acids (BNAs). A bridged nucleic acid is a nucleotide bearing a conformationally restricted sugar moiety. Oligonucleotides containing BNAs show high binding affinity with RNA complementary strands, and are more tolerant to endonucleolytic and exonucleolytic degradation (Roongjang, S. et al., (2007) Nucleic Acids Symp Ser (Oxf) 51:113-114). Exemplary BNAs include, but are not limited to 2′4′-BNA (also known as LNA (see below); 3′-amino2′,4′-BNA, 3′,4′-BNA; BNA^{COC}; BNA^{NC}, and BNA^{(ME)}. The structure of the BNA will influence the binding affinity of the nucleic acid molecule with complementary single stranded DNA and double stranded DNA, as well as its enzymatic stability against nuclease degradation. The synthesis and purification of BNA molecules can be performed using standard protocols, (e.g., see Imanishi T, et al., (2002) Chem. Commun. 16: 1653-1659).

In some embodiments, the nucleic acid can be modified to generate peptide nucleic acids (see Hyrup B. et al. (1996) Bioorganic & Medicinal Chemistry 4: 5-23). As used herein, the terms “peptide nucleic acid” or “PNA” refers to a nucleic acid mimic, e.g., a DNA or RNA mimic, in which the deoxyribose or ribose phosphate backbone is replaced by a pseudopeptide backbone and only the four natural nucleobases are retained. The neutral backbone of a PNA can allow for specific hybridization to DNA and RNA under conditions of low ionic strength. PNAs of nucleic acid fusion inhibitor molecules can be used in therapeutic and diagnostic applications. For example, PNAs can be used as antisense, antigene, siRNA, or RNAi agents for sequence-specific modulation of gene expression by, for example, inducing transcription or translation arrest or inhibiting replication. PNAs of nucleic acid fusion inhibitor molecules can also be used in the analysis of single base pair mutations in a gene, (e.g., by PNA-directed PCR clamping); as ‘artificial restriction enzymes’ when used in combination with other enzymes, (e.g., SI nucleases (Hyrup B. et al. (1996) supra)); or as probes or primers for DNA sequencing or hybridization (Hyrup B. et al. (1996) supra; Perry-O'Keefe supra).

The synthesis of PNA oligomers can be performed using standard solid phase peptide synthesis protocols as described in Hyrup B. et al. (1996) supra and Perry-O'Keefe et al. Proc. Natl. Acad. Sci. 93: 14670-675. Representative U.S. patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in RNA molecules are described in, for example, in Nielsen et al., Science, 1991, 254, 1497-1500.

The nucleic acid fusion inhibitor molecules can also be modified to include one or more locked nucleic acids (LNA). A locked nucleic acid is a nucleotide having a modified sugar moiety in which the sugar moiety comprises an extra bridge connecting the 2′ and 4′ carbons. This structure effectively “locks” the ribose in the 3′-endo structural conformation. LNA containing nucleic acid molecules possess high affinity to complementary DNA and RNA and improved mismatch discrimination relative to unmodified nucleic acid molecules (Jepson. J., et al., (2004) Oligonucleotides 14:130-146). The addition of locked nucleic acids to siRNAs has been shown to increase siRNA stability in serum, and to reduce off-target effects (Elmen, J. et al., (2005) Nucleic Acids Research 33(1):439-447; Mook. O R. et al., (2007) Mol Canc Ther 6(3):833-843; Grunweller, A. et al., (2003) Nucleic Acids Research 31(12):3185-3193). Representative U.S. Patents that teach the preparation of locked nucleic acid nucleotides include, but are not limited to, the following: U.S. Pat. Nos. 6,268,490; 6,670,461; 6,794,499; 6,998,484; 7,053,207; 7,084,125; and 7,399,845, the entire contents of each of which are hereby incorporated herein by reference.

A nucleic acid fusion inhibitor molecule can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T, and Lebleu. B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine, 5-methylcytosine substitutions have been shown to increase nucleic acid duplex stability by 0.6-1.2° C. (Sanghvi, Y. S., Crooke, S. T, and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681.941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

Potentially stabilizing modifications to the ends of nucleic acid fusion inhibitor molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-0-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

In other embodiments, the nucleic acid fusion inhibitor molecule may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; WO88/09810) or the blood-brain barrier (see, e.g., WO 89/10134). In addition, oligonucleotides can be modified with hybridization-triggered cleavage agents (see, e.g., Krol et al. (1988) Bio-Techniques 6:958-976) or intercalating agents (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

In some embodiment, modifications to the fusion nucleic acid molecules can include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; and/or backbone modifications, including modification or replacement of the phosphodiester linkages. Specific examples include, but are not limited to fusion nucleic acid molecules containing modified backbones or no natural internucleoside linkages, fusion nucleic acid molecules having modified backbones include, among others, those that do not have a phosphorus atom in the backbone.

Modified nucleic acid backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′.

Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and US Pat RE39464, the entire contents of each of which are hereby incorporated herein by reference.

Modified nucleic acid backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, the entire contents of each of which are hereby incorporated herein by reference.

Some embodiments include nucleic acid fusion inhibitor molecules with phosphorothioate backbones and oligonucleosides with heteroatom backbones, and in particular —CH₂—NH—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂— and —N(CH₃)—CH₂—CH₂—[wherein the native phosphodiester backbone is represented as —O—P—O—CH₂—] of the above-referenced U.S. Pat. No. 5,489,677, and the amide backbones of the above-referenced U.S. Pat. No. 5,602,240.

Modified nucleic acid fusion inhibitor molecules can also contain one or more substituted sugar moieties. The nucleic acid, e.g., RNA, molecules can include one of the following at the 2′-position: OIH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an RNA molecule, or a group for improving the pharmacodynamic properties of an RNA molecule, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH—N(CH₂)₂.

Other modifications can include 2′-methoxy (2′-OCH₃), 2′-aminopropoxy (2′-OCH₂Cl₂CH₂NH₂) and 2′-fluoro (2′-F). Similar modifications can also be made at other positions on the RNA of an RNA molecule, particularly the 3′ position of the sugar on the 3′ terminal nucleotide or in 2′-5′ linked dsRNAs and the 5′ position of 5′ terminal nucleotide. RNA molecules can also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application. The entire contents of each of the foregoing are hereby incorporated herein by reference. Evaluation of Subjects

Subjects, e.g., patients, can be evaluated for the presence of a fusion molecule described herein. A patient can be evaluated, for example, by determining the genomic sequence of the patient, e.g., by an NGS method. Alternatively, or in addition, evaluation of a patient can include directly assaying for the presence of a fusion described herein, in the patient, such as by an assay to detect a fusion nucleic acid (e.g., DNA or RNA), such as by, Southern blot, Northern blot, or RT-PCR, e.g., qRT-PCR. Alternatively, or in addition, a patient can be evaluated for the presence of a protein fusion, such as by immunohistochemistry, Western blot, immunoprecipitation, or immunomagnetic bead assay.

Evaluation of a patient can also include a cytogenetic assay, such as by fluorescence in situ hybridization (FISH), to identify the chromosomal rearrangement resulting in the fusion. For example, to perform FISH, at least a first probe tagged with a first detectable label can be designed to target CEP89, such as in one or more exons of CEP89 and at least a second probe tagged with a second detectable label can be designed to target BRAF, such as in one or more exons of BRAF (e.g., the exons containing the part of the protein that includes the tyrosine kinase domain). The at least one first probe and the at least one second probe will be closer together in patients who carry the CEP89-BRAF fusion than in patients who do not carry the CEP89-BRAF fusion. These methods can be utilized in a similar manner for any fusion described herein.

Additional methods for fusion detection are provided below.

In one aspect, the results of a clinical trial, e.g., a successful or unsuccessful clinical trial, can be repurposed to identify agents that target a fusion described herein. By one exemplary method, a candidate agent used in a clinical trial can be reevaluated to determine if the agent in the trial targets a fusion, or is effective to treat a tumor containing a particular fusion. For example, subjects who participated in a clinical trial for an agent, such as a kinase inhibitor, can be identified. Patients who experienced an improvement in symptoms, e.g., cancer (e.g., lung cancer) symptoms, such as decreased tumor size, or decreased rate of tumor growth, can be evaluated for the presence of a fusion described herein. Patients who did not experience an improvement in cancer symptoms can also be evaluated for the presence of a fusion described herein. Where patients carrying a fusion described herein are found to have been more likely to respond to the test agent than patients who did not carry such a fusion, then the agent is determined to be an appropriate treatment option for a patient carrying the fusion.

“Reevaluation” of patients can include, for example, determining the genomic sequence of the patients, or a subset of the clinical trial patients, e.g., by an NGS method. Alternatively, or in addition, reevaluation of the patients can include directly assaying for the presence of a fusion described herein, in the patient, such as by an assay to detect a fusion nucleic acid (e.g., RNA), such as by RT-PCR, e.g., qRT-PCR. Alternatively, or in addition, a patient can be evaluated for the presence of a protein fusion, such as by immunohistochemistry, Western blot, immunoprecipitation, or immunomagnetic bead assay.

Clinical trials suitable for repurposing as described above include trials that tested tyrosine kinase inhibitors, and multikinase inhibitors.

### Methods for Detection of Fusion Nucleic Acids and Polypeptides

Methods for evaluating a fusion gene, mutations and/or gene products are known to those of skill in the art. In one embodiment, the fusion is detected in a nucleic acid molecule by a method chosen from one or more of: nucleic acid hybridization assay, amplification-based assays (e.g., polymerase chain reaction (PCR)), PCR-RFLP assay, real-time PCR, sequencing, screening analysis (including metaphase cytogenetic analysis by standard karyotype methods, FISH (e.g., break away FISH), spectral karyotyping or MFISH, comparative genomic hybridization), in situ hybridization, SSP, HPLC or mass-spectrometric genotyping.

Additional exemplary methods include, traditional “direct probe” methods such as Southern blots or in situ hybridization (e.g., fluorescence in situ hybridization (FISH) and FISH plus SKY), and “comparative probe” methods such as comparative genomic hybridization (CGH), e.g., cDNA-based or oligonucleotide-based CGH, can be used. The methods can be used in a wide variety of formats including, but not limited to, substrate (e.g., membrane or glass) bound methods or array-based approaches.

In certain embodiments, the evaluation methods include the probes/primers described herein. In one embodiment, probes/primers can be designed to detect a fusion molecule described herein or a reciprocal thereof. Probes/primers are suitable, e.g., for FISH or PCR amplification. For PCR, e.g., to amply a region including a fusion junction described herein, forward primers can be designed to hybridize to a gene sequence from nucleotides corresponding to one of the genes of a fusion described herein, and reverse primers can be designed to hybridize to a sequence from nucleotides corresponding to the second gene involved in the fusion.

For example, probes/primers can be designed to detect a CEP89-BRAF fusion or a reciprocal thereof. The BRAF probes/primers can hybridize to the nucleotides encoding one or more exons of the FGFR3 protein. The CEP89 probes/primers can hybridize to the nucleotides encoding one or more exons of the CEP89 protein). These probes/primers are suitable, e.g., for FISH or PCR amplification.

The probes/primers described above use CEP89-BRAF as an example, and such methods can be readily applied to any of the fusions described herein by one of skill in the art.

In one embodiment, FISH analysis is used to identify the chromosomal rearrangement resulting in the fusions as described above. For example, to perform FISH, at least a first probe tagged with a first detectable label can be designed to target a first gene of a fusion described herein, such as in one or more exons of the gene and at least a second probe tagged with a second detectable label can be designed to target a second gene of the fusion, such as in one or more exons of genes (e.g., the exons containing the part of the protein that includes the tyrosine kinase domain). The at least one first probe and the at least one second probe will be closer together in a subject who carries the fusion compared to a subject who does not carry the fusion.

In one approach, a variation of a FISH assay, e.g., “break-away FISH”, is used to evaluate a patient. By this method, at least one probe targeting the fusion junction and at least one probe targeting an individual gene of the fusion, e.g., at one or more exons and or introns of the gene, are utilized. In normal cells, both probes will be observed (or a secondary color will be observed due to the close proximity of the two genes of the gene fusion), and only the single gene probe will be observed when the translocation occurs. Other variations of the FISH method known in the art are suitable for evaluating a patient.

For example, by this method, at least one probe targeting the BRAF intron 8/CEP89 intron 16 junction and at least one probe targeting CEP89 (or BRAF), e.g., at one or more exons and or introns of CEP89 or BRAF, are utilized. In normal cells, both probes will be observed (or a secondary color will be observed due to the close proximity of the CEP89 and BRAF genes), and only the CEP89 probe will be observed when the translocation occurs. Other variations of the FISH method known in the art are suitable for evaluating a patient.

The FISH methods described herein above use CEP89-BRAF as an example, and such methods can be readily applied to any of the fusions described herein by one of skill in the art.

Probes are used that contain DNA segments that are essentially complementary to DNA base sequences existing in different portions of chromosomes. Examples of probes useful according to the invention, and labeling and hybridization of probes to samples are described in two U.S. patents to Vysis, Inc. U.S. Pat. Nos. 5,491,224 and 6,277,569 to Bittner, et al.

Additional protocols for FISH detection are described below.

Chromosomal probes are typically about 50 to about 10³ nucleotides in length. Longer probes typically comprise smaller fragments of about 100 to about 500 nucleotides in length. Probes that hybridize with centromeric DNA and locus-specific DNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, chromosome (e.g., human chromosome) along with the normal chromosome complement of the host, and chromosomes purified by flow cytometry or microdissection. The region of interest can be isolated through cloning, or by site-specific amplification via the polymerase chain reaction (PCR). See, for example, Nath and Johnson, Biotechnic Histochem., 1998, 73 (1):6-22, Wheeless et al., Cytometry 1994, 17:319-326, and U.S. Pat. No. 5,491,224.

The probes to be used hybridize to a specific region of a chromosome to determine whether a cytogenetic abnormality is present in this region. One type of cytogenetic abnormality is a deletion. Although deletions can be of one or more entire chromosomes, deletions normally involve loss of part of one or more chromosomes. If the entire region of a chromosome that is contained in a probe is deleted from a cell, hybridization of that probe to the DNA from the cell will normally not occur and no signal will be present on that chromosome. If the region of a chromosome that is partially contained within a probe is deleted from a cell, hybridization of that probe to the DNA from the cell can still occur, but less of a signal can be present. For example, the loss of a signal is compared to probe hybridization to DNA from control cells that do not contain the genetic abnormalities which the probes are intended to detect. In some embodiments, at least 1, 5, 10, 20, 30, 40, 50, 60, 70, 80, 90, 100, 110, 120, 130, 140, 150, 160, 170, 180, 190, 200, or more cells are enumerated for presence of the cytogenetic abnormality.

Cytogenetic abnormalities to be detected can include, but are not limited to, non-reciprocal translocations, balanced translocations, intra-chromosomal inversions, point mutations, deletions, gene copy number changes, gene expression level changes, and germ line mutations. In particular, one type of cytogenetic abnormality is a duplication. Duplications can be of entire chromosomes, or of regions smaller than an entire chromosome. If the region of a chromosome that is contained in a probe is duplicated in a cell, hybridization of that probe to the DNA from the cell will normally produce at least one additional signal as compared to the number of signals present in control cells with no abnormality of the chromosomal region contained in the probe.

Chromosomal probes are labeled so that the chromosomal region to which they hybridize can be detected. Probes typically are directly labeled with a fluorophore, an organic molecule that fluoresces after absorbing light of lower wavelength/higher energy. The fluorophore allows the probe to be visualized without a secondary detection molecule. After covalently attaching a fluorophore to a nucleotide, the nucleotide can be directly incorporated into the probe with standard techniques such as nick translation, random priming, and PCR labeling. Alternatively, deoxycytidine nucleotides within the probe can be transaminated with a linker. The fluorophore then is covalently attached to the transaminated deoxycytidine nucleotides. See, U.S. Pat. No. 5,491,224.

U.S. Pat. No. 5,491,224 describes probe labeling as a number of the cytosine residues having a fluorescent label covalently bonded thereto. The number of fluorescently labeled cytosine bases is sufficient to generate a detectable fluorescent signal while the individual so labeled DNA segments essentially retain their specific complementary binding (hybridizing) properties with respect to the chromosome or chromosome region to be detected. Such probes are made by taking the unlabeled DNA probe segment, transaminating with a linking group a number of deoxycytidine nucleotides in the segment, covalently bonding a fluorescent label to at least a portion of the transaminated deoxycytidine bases.

Probes can also be labeled by nick translation, random primer labeling or PCR labeling. Labeling is done using either fluorescent (direct)- or haptene (indirect)-labeled nucleotides. Representative, non-limiting examples of labels include: AMCA-6-dUTP, CascadeBlue-4-dUTP, Fluorescein-12-dUTP, Rhodamine-6-dUTP, TexasRed-6-dUTP, Cy3-6-dUTP, Cy5-dUTP, Biotin(BIO)-11-dUTP, Digoxygenin(DIG)-11-dUTP or Dinitrophenyl (DNP)-11-dUTP.

Probes also can be indirectly labeled with biotin or digoxygenin, or labeled with radioactive isotopes such as ³²p and ³H, although secondary detection molecules or further processing then is required to visualize the probes. For example, a probe labeled with biotin can be detected by avidin conjugated to a detectable marker. For example, avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Probes can also be prepared such that a fluorescent or other label is not part of the DNA before or during the hybridization, and is added after hybridization to detect the probe hybridized to a chromosome. For example, probes can be used that have antigenic molecules incorporated into the DNA. After hybridization, these antigenic molecules are detected using specific antibodies reactive with the antigenic molecules. Such antibodies can themselves incorporate a fluorochrome, or can be detected using a second antibody with a bound fluorochrome.

However treated or modified, the probe DNA is commonly purified in order to remove unreacted, residual products (e.g., fluorochrome molecules not incorporated into the DNA) before use in hybridization.

Prior to hybridization, chromosomal probes are denatured according to methods well known in the art. Probes can be hybridized or annealed to the chromosomal DNA under hybridizing conditions. “Hybridizing conditions” are conditions that facilitate annealing between a probe and target chromosomal DNA. Since annealing of different probes will vary depending on probe length, base concentration and the like, annealing is facilitated by varying probe concentration, hybridization temperature, salt concentration and other factors well known in the art.

Hybridization conditions are facilitated by varying the concentrations, base compositions, complexities, and lengths of the probes, as well as salt concentrations, temperatures, and length of incubation. For example, in situ hybridizations are typically performed in hybridization buffer containing 1-2×SSC, 50-65% formamide and blocking DNA to suppress non-specific hybridization. In general, hybridization conditions, as described above, include temperatures of about 25° C. to about 55° C., and incubation lengths of about 0.5 hours to about 96 hours.

Non-specific binding of chromosomal probes to DNA outside of the target region can be removed by a series of washes. Temperature and concentration of salt in each wash are varied to control stringency of the washes. For example, for high stringency conditions, washes can be carried out at about 65° C. to about 80° C., using 0.2× to about 2×SSC, and about 0.1% to about 1% of a non-ionic detergent such as Nonidet P-40 (NP40). Stringency can be lowered by decreasing the temperature of the washes or by increasing the concentration of salt in the washes. In some applications it is necessary to block the hybridization capacity of repetitive sequences. Thus, in some embodiments, tRNA, human genomic DNA, or Cot-I DNA is used to block non-specific hybridization. After washing, the slide is allowed to drain and air dry, then mounting medium, a counterstain such as DAPI, and a coverslip are applied to the slide. Slides can be viewed immediately or stored at −20° C., before examination.

For fluorescent probes used in fluorescence in situ hybridization (FISH) techniques, fluorescence can be viewed with a fluorescence microscope equipped with an appropriate filter for each fluorophore, or by using dual or triple band-pass filter sets to observe multiple fluorophores. See, for example, U.S. Pat. No. 5,776,688. Alternatively, techniques such as flow cytometry can be used to examine the hybridization pattern of the chromosomal probes.

In CGH methods, a first collection of nucleic acids (e.g., from a sample, e.g., a possible tumor) is labeled with a first label, while a second collection of nucleic acids (e.g., a control, e.g., from a healthy cell/tissue) is labeled with a second label. The ratio of hybridization of the nucleic acids is determined by the ratio of the two (first and second) labels binding to each fiber in the array. Where there are chromosomal deletions or multiplications, differences in the ratio of the signals from the two labels will be detected and the ratio will provide a measure of the copy number. Array-based CGH can also be performed with single-color labeling (as opposed to labeling the control and the possible tumor sample with two different dyes and mixing them prior to hybridization, which will yield a ratio due to competitive hybridization of probes on the arrays). In single color CGH, the control is labeled and hybridized to one array and absolute signals are read, and the possible tumor sample is labeled and hybridized to a second array (with identical content) and absolute signals are read. Copy number difference is calculated based on absolute signals from the two arrays.

Hybridization protocols suitable for use with the methods featured in the invention are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In situ Hybridization Protocols, Choo, ed., Humana Press, Totowa, N.J. (1994), etc. In one embodiment, the hybridization protocol of Pinkel, et al. (1998) Nature Genetics 20: 207-211, or of Kallioniemi (1992) Proc. Natl Acad. Sci USA 89:5321-5325 (1992) is used. Array-based CGH is described in U.S. Pat. No. 6,455,258, the contents of each of which are incorporated herein by reference.

In still another embodiment, amplification-based assays can be used to measure presence/absence and copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g., healthy tissue, provides a measure of the copy number.

Methods of “quantitative” amplification are well known to those of skill in the art. For example, quantitative PCR involves simultaneously co-amplifying a known quantity of a control sequence using the same primers. This provides an internal standard that can be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR can also be used. In fluorogenic quantitative PCR, quantitation is based on amount of fluorescence signals. e.g., TaqMan and sybr green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc.

### Nucleic Acid Samples

A variety of tissue samples can be the source of the nucleic acid samples used in the present methods. Genomic or subgenomic DNA fragments can be isolated from a subject's sample (e.g., a tumor sample, a normal adjacent tissue (NAT), a blood sample or any normal control)). In certain embodiments, the tissue sample is preserved as a frozen sample or as formaldehyde- or paraformaldehyde-fixed paraffin-embedded (FFPE) tissue preparation. For example, the sample can be embedded in a matrix, e.g., an FFPE block or a frozen sample. The isolating step can include flow-sorting of individual chromosomes; and/or micro-dissecting a subject's sample (e.g., a tumor sample, a NAT, a blood sample).

Protocols for DNA isolation from a tissue sample are known in the art. Additional methods to isolate nucleic acids (e.g., DNA) from formaldehyde- or paraformaldehyde-fixed, paraffin-embedded (FFPE) tissues are disclosed, e.g., in Cronin M. et al., (2004) Am J Pathol. 164(1):35-42; Masuda N. et al., (1999) Nucleic Acids Res. 27(22):4436-4443; Specht K. et al., (2001) Am J Pathol. 158(2):419-429, Ambion RecoverAll™ Total Nucleic Acid Isolation Protocol (Ambion, Cat. No. AM1975, September 2008), and QIAamp® DNA FFPE Tissue Handbook (Qiagen, Cat. No. 37625, October 2007). RecoverAll™ Total Nucleic Acid Isolation Kit uses xylene at elevated temperatures to solubilize paraffin-embedded samples and a glass-fiber filter to capture nucleic acids. QIAamp® DNA FFPE Tissue Kit uses QIAamp® DNA Micro technology for purification of genomic and mitochondrial DNA.

The isolated nucleic acid samples (e.g., genomic DNA samples) can be fragmented or sheared by practicing routine techniques. For example, genomic DNA can be fragmented by physical shearing methods, enzymatic cleavage methods, chemical cleavage methods, and other methods well known to those skilled in the art. The nucleic acid library can contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library is a portion of the genome, i.e., where the complexity of the genome is reduced by design. In some embodiments, any selected portion of the genome can be used with the methods described herein. In certain embodiments, the entire exome or a subset thereof is isolated.

Methods can further include isolating a nucleic acid sample to provide a library (e.g., a nucleic acid library). In certain embodiments, the nucleic acid sample includes whole genomic, subgenomic fragments, or both. The isolated nucleic acid samples can be used to prepare nucleic acid libraries. Thus, in one embodiment, the methods featured in the invention further include isolating a nucleic acid sample to provide a library (e.g., a nucleic acid library as described herein). Protocols for isolating and preparing libraries from whole genomic or subgenomic fragments are known in the art (e.g., Illumina's genomic DNA sample preparation kit). In certain embodiments, the genomic or subgenomic DNA fragment is isolated from a subject's sample (e.g., a tumor sample, a normal adjacent tissue (NAT), a blood sample or any normal control)). In one embodiment, the sample (e.g., the tumor or NAT sample) is a preserved. For example, the sample is embedded in a matrix, e.g., an FFPE block or a frozen sample. In certain embodiments, the isolating step includes flow-sorting of individual chromosomes; and/or microdissecting a subject's sample (e.g., a tumor sample, a NAT, a blood sample). In certain embodiments, the nucleic acid sample used to generate the nucleic acid library is less than 5, less than 1 microgram, less than 500 ng, less than 200 ng, less than 100 ng, less than 50 ng or less than 20 ng (e.g., 10 ng or less).

In still other embodiments, the nucleic acid sample used to generate the library includes RNA or cDNA derived from RNA. In some embodiments, the RNA includes total cellular RNA. In other embodiments, certain abundant RNA sequences (e.g., ribosomal RNAs) have been depleted. In some embodiments, the poly(A)-tailed mRNA fraction in the total RNA preparation has been enriched. In some embodiments, the cDNA is produced by random-primed cDNA synthesis methods. In other embodiments, the cDNA synthesis is initiated at the poly(A) tail of mature mRNAs by priming by oligo(dT)-containing oligonucleotides. Methods for depletion, poly(A) enrichment, and cDNA synthesis are well known to those skilled in the art.

The method can further include amplifying the nucleic acid sample (e.g., DNA or RNA sample) by specific or non-specific nucleic acid amplification methods that are well known to those skilled in the art. In some embodiments, certain embodiments, the nucleic acid sample is amplified, e.g., by whole-genome amplification methods such as random-primed strand-displacement amplification.

In other embodiments, the nucleic acid sample is fragmented or sheared by physical or enzymatic methods and ligated to synthetic adapters, size-selected (e.g., by preparative gel electrophoresis) and amplified (e.g., by PCR). In other embodiments, the fragmented and adapter-ligated group of nucleic acids is used without explicit size selection or amplification prior to hybrid selection.

In other embodiments, the isolated DNA (e.g., the genomic DNA) is fragmented or sheared. In some embodiments, the library includes less than 50% of genomic DNA, such as a subfraction of genomic DNA that is a reduced representation or a defined portion of a genome, e.g., that has been subfractionated by other means. In other embodiments, the library includes all or substantially all genomic DNA.

In some embodiments, the library includes less than 50% of genomic DNA, such as a subfraction of genomic DNA that is a reduced representation or a defined portion of a genome, e.g., that has been subfractionated by other means. In other embodiments, the library includes all or substantially all genomic DNA. Protocols for isolating and preparing libraries from whole genomic or subgenomic fragments are known in the art (e.g., Illumina's genomic DNA sample preparation kit). Alternative DNA shearing methods can be more automatable and/or more efficient (e.g., with degraded FFPE samples). Alternatives to DNA shearing methods can also be used to avoid a ligation step during library preparation.

The methods described herein can be performed using a small amount of nucleic acids, e.g., when the amount of source DNA is limiting (e.g., even after whole-genome amplification). In one embodiment, the nucleic acid comprises less than about 5 μg, 4 μg, 3 μg, 2 μg, 1 μg, 0.8 μg, 0.7 μg, 0.6 μg, 0.5 μg, or 400 ng, 300 ng, 200 ng, 100 ng, 50 ng, or 20 ng or less of nucleic acid sample. For example, to prepare 500 ng of hybridization-ready nucleic acids, one typically begins with 3 μg of genomic DNA. One can start with less, however, if one amplifies the genomic DNA (e.g., using PCR) before the step of solution hybridization. Thus it is possible, but not essential, to amplify the genomic DNA before solution hybridization.

In some embodiments, a library is generated using DNA (e.g., genomic DNA) from a sample tissue, and a corresponding library is generated with RNA (or cDNA) isolated from the same sample tissue.

### Design of Baits

A bait can be a nucleic acid molecule, e.g., a DNA or RNA molecule, which can hybridize to (e.g., be complementary to), and thereby allow capture of a target nucleic acid. In one embodiment, a bait is an RNA molecule. In other embodiments, a bait includes a binding entity, e.g., an affinity tag, that allows capture and separation, e.g., by binding to a binding entity, of a hybrid formed by a bait and a nucleic acid hybridized to the bait. In one embodiment, a bait is suitable for solution phase hybridization.

Baits can be produced and used by methods and hybridization conditions as described in US 2010/0029498 and Gnirke, A. et al. (2009) Nat Biotechnol. 27(2):182-189, and U.S. Ser. No. 61/428,568, filed Dec. 30, 2010, incorporated herein by reference. For example, biotinylated RNA baits can be produced by obtaining a pool of synthetic long oligonucleotides, originally synthesized on a microarray, and amplifying the oligonucleotides to produce the bait sequences. In some embodiments, the baits are produced by adding an RNA polymerase promoter sequence at one end of the bait sequences, and synthesizing RNA sequences using RNA polymerase. In one embodiment, libraries of synthetic oligodeoxynucleotides can be obtained from commercial suppliers, such as Agilent Technologies, Inc., and amplified using known nucleic acid amplification methods.

Each bait sequence can include a target-specific (e.g., a member-specific) bait sequence and universal tails on each end. As used herein, the term “bait sequence” can refer to the target-specific bait sequence or the entire oligonucleotide including the target-specific “bait sequence” and other nucleotides of the oligonucleotide. In one embodiment, a target-specific bait hybridizes to a nucleic acid sequence comprising a nucleic acid sequence in an intron of one gene of a fusion described herein, in an intron of the other gene of a fusion described herein, or a fusion junction joining the introns. In one embodiment, the bait is an oligonucleotide about 200 nucleotides in length, of which 170 nucleotides are target-specific “bait sequence”. The other 30 nucleotides (e.g., 15 nucleotides on each end) are universal arbitrary tails used for PCR amplification. The tails can be any sequence selected by the user.

The bait sequences described herein can be used for selection of exons and short target sequences. In one embodiment, the bait is between about 100 nucleotides and 300 nucleotides in length. In another embodiment, the bait is between about 130 nucleotides and 230 nucleotides in length. In yet another embodiment, the bait is between about 150 nucleotides and 200 nucleotides in length. The target-specific sequences in the baits, e.g., for selection of exons and short target sequences, are between about 40 nucleotides and 1000 nucleotides in length. In one embodiment, the target-specific sequence is between about 70 nucleotides and 300 nucleotides in length. In another embodiment, the target-specific sequence is between about 100 nucleotides and 200 nucleotides in length. In yet another embodiment, the target-specific sequence is between about 120 nucleotides and 170 nucleotides in length.

### Sequencing

The invention also includes methods of sequencing nucleic acids. In one embodiment, any of a variety of sequencing reactions known in the art can be used to directly sequence at least a portion of a fusion molecule described herein. In one embodiment, the fusion sequence is compared to a corresponding reference (control) sequence.

In one embodiment, the sequence of the fusion nucleic acid molecule is determined by a method that includes one or more of: hybridizing an oligonucleotide, e.g., an allele specific oligonucleotide for one alteration described herein to said nucleic acid, hybridizing a primer, or a primer set (e.g., a primer pair), that amplifies a region comprising the mutation or a fusion junction of the allele; amplifying, e.g., specifically amplifying, a region comprising the mutation or a fusion junction of the allele; attaching an adapter oligonucleotide to one end of a nucleic acid that comprises the mutation or a fusion junction of the allele; generating an optical, e.g., a colorimetric signal, specific to the presence of the one of the mutation or fusion junction; hybridizing a nucleic acid comprising the mutation or fusion junction to a second nucleic acid, e.g., a second nucleic acid attached to a substrate; generating a signal, e.g., an electrical or fluorescent signal, specific to the presence of the mutation or fusion junction; and incorporating a nucleotide into an oligonucleotide that is hybridized to a nucleic acid that contains the mutation or fusion junction.

In another embodiment, the sequence is determined by a method that comprises one or more of: determining the nucleotide sequence from an individual nucleic acid molecule, e.g., where a signal corresponding to the sequence is derived from a single molecule as opposed, e.g., from a sum of signals from a plurality of clonally expanded molecules; determining the nucleotide sequence of clonally expanded proxies for individual nucleic acid molecules; massively parallel short-read sequencing: template-based sequencing; pyrosequencing; real-time sequencing comprising imaging the continuous incorporation of dye-labeling nucleotides during DNA synthesis; nanopore sequencing; sequencing by hybridization; nano-transistor array based sequencing; polony sequencing; scanning tunneling microscopy (STM) based sequencing; or nanowire-molecule sensor based sequencing.

Any method of sequencing known in the art can be used. Exemplary sequencing reactions include those based on techniques developed by Maxam and Gilbert (Proc. Natl Acad. Sci USA (1977) 74:560) or Sanger (Sanger et al. (1977) Proc. Nat. Acad. Sci 74:5463). Any of a variety of automated sequencing procedures can be utilized when performing the assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Köster; U.S. Pat. No. 5,547,835 and international patent application Publication Number WO 94/21822 entitled DNA Sequencing by Mass Spectrometry Via Exonuclease Degradation by H. Köster), and U.S. Pat. No. 5,605,798 and International Patent Application No. PCT/US96/03651 entitled DNA Diagnostics Based on Mass Spectrometry by H. Köster; Cohen et al. (1996) Adv Chromatogr 36:127-162; and Griffin et al. (1993) Appl Biochem Biotechnol 38: 147-159).

Sequencing of nucleic acid molecules can also be carried out using next-generation sequencing (NGS). Next-generation sequencing includes any sequencing method that determines the nucleotide sequence of either individual nucleic acid molecules or clonally expanded proxies for individual nucleic acid molecules in a highly parallel fashion (e.g., greater than 10³ molecules are sequenced simultaneously). In one embodiment, the relative abundance of the nucleic acid species in the library can be estimated by counting the relative number of occurrences of their cognate sequences in the data generated by the sequencing experiment. Next generation sequencing methods are known in the art, and are described, e.g., in Metzker, M. (2010) Nature Biotechnology Reviews 11:31-46, incorporated herein by reference.

In one embodiment, the next-generation sequencing allows for the determination of the nucleotide sequence of an individual nucleic acid molecule (e.g., Helicos BioSciences' HeliScope Gene Sequencing system, and Pacific Biosciences' PacBio RS system). In other embodiments, the sequencing method determines the nucleotide sequence of clonally expanded proxies for individual nucleic acid molecules (e.g., the Solexa sequencer, Illumina Inc., San Diego, Calif.; 454 Life Sciences (Branford, Conn.), and Ion Torrent). e.g., massively parallel short-read sequencing (e.g., the Solexa sequencer, Illumina Inc., San Diego, Calif.), which generates more bases of sequence per sequencing unit than other sequencing methods that generate fewer but longer reads. Other methods or machines for next-generation sequencing include, but are not limited to, the sequencers provided by 454 Life Sciences (Branford, Conn.), Applied Biosystems (Foster City, Calif.; SOLiD sequencer), and Helicos BioSciences Corporation (Cambridge, Mass.).

Platforms for next-generation sequencing include, but are not limited to, Roche/454's Genome Sequencer (GS) FLX System, Illumina/Solexa's Genome Analyzer (GA), Life/APG's Support Oligonucleotide Ligation Detection (SOLiD) system, Polonator's G.007 system, Helicos BioSciences' HeliScope Gene Sequencing system, and Pacific Biosciences' PacBio RS system.

NGS technologies can include one or more of steps, e.g., template preparation, sequencing and imaging, and data analysis.

### Template Preparation

Methods for template preparation can include steps such as randomly breaking nucleic acids (e.g., genomic DNA or cDNA) into smaller sizes and generating sequencing templates (e.g., fragment templates or mate-pair templates). The spatially separated templates can be attached or immobilized to a solid surface or support, allowing massive amounts of sequencing reactions to be performed simultaneously. Types of templates that can be used for NGS reactions include, e.g., clonally amplified templates originating from single DNA molecules, and single DNA molecule templates.

Methods for preparing clonally amplified templates include, e.g., emulsion PCR (emPCR) and solid-phase amplification.

EmPCR can be used to prepare templates for NGS. Typically, a library of nucleic acid fragments is generated, and adapters containing universal priming sites are ligated to the ends of the fragment. The fragments are then denatured into single strands and captured by beads. Each bead captures a single nucleic acid molecule. After amplification and enrichment of emPCR beads, a large amount of templates can be attached or immobilized in a polyacrylamide gel on a standard microscope slide (e.g., Polonator), chemically crosslinked to an amino-coated glass surface (e.g., Life/APG; Polonator), or deposited into individual PicoTiterPlate (PTP) wells (e.g., Roche/454), in which the NGS reaction can be performed.

Solid-phase amplification can also be used to produce templates for NGS. Typically, forward and reverse primers are covalently attached to a solid support. The surface density of the amplified fragments is defined by the ratio of the primers to the templates on the support. Solid-phase amplification can produce hundreds of millions spatially separated template clusters (e.g., Illumina/Solexa). The ends of the template clusters can be hybridized to universal sequencing primers for NGS reactions.

Other methods for preparing clonally amplified templates also include, e.g., Multiple Displacement Amplification (MDA) (Lasken R. S. Curr Opin Microbiol. 2007; 10(5):510-6). MDA is a non-PCR based DNA amplification technique. The reaction involves annealing random hexamer primers to the template and DNA synthesis by high fidelity enzyme, typically (29 at a constant temperature. MDA can generate large sized products with lower error frequency.

Template amplification methods such as PCR can be coupled with NGS platforms to target or enrich specific regions of the genome (e.g., exons). Exemplary template enrichment methods include, e.g., microdroplet PCR technology (Tewhey R. et al., Nature Biotech. 2009, 27:1025-1031), custom-designed oligonucleotide microarrays (e.g., Roche/NimbleGen oligonucleotide microarrays), and solution-based hybridization methods (e.g., molecular inversion probes (MIPs) (Porreca G. J. et al., Nature Methods, 2007, 4:931-936; Krishnakumar S. et al., Proc. Natl. Acad. Sci. USA, 2008, 105:9296-9310; Turner E. H. et al., Nature Methods, 2009, 6:315-316), and biotinylated RNA capture sequences (Gnirke A. et al., Nat. Biotechnol. 2009; 27(2): 182-9)

Single-molecule templates are another type of templates that can be used for NGS reaction. Spatially separated single molecule templates can be immobilized on solid supports by various methods. In one approach, individual primer molecules are covalently attached to the solid support. Adapters are added to the templates and templates are then hybridized to the immobilized primers. In another approach, single-molecule templates are covalently attached to the solid support by priming and extending single-stranded, single-molecule templates from immobilized primers. Universal primers are then hybridized to the templates. In yet another approach, single polymerase molecules are attached to the solid support, to which primed templates are bound.

### Sequencing and Imaging

Exemplary sequencing and imaging methods for NGS include, but are not limited to, cyclic reversible termination (CRT), sequencing by ligation (SBL), single-molecule addition (pyrosequencing), and real-time sequencing.

CRT uses reversible terminators in a cyclic method that minimally includes the steps of nucleotide incorporation, fluorescence imaging, and cleavage. Typically, a DNA polymerase incorporates a single fluorescently modified nucleotide corresponding to the complementary nucleotide of the template base to the primer. DNA synthesis is terminated after the addition of a single nucleotide and the unincorporated nucleotides are washed away. Imaging is performed to determine the identity of the incorporated labeled nucleotide. Then in the cleavage step, the terminating/inhibiting group and the fluorescent dye are removed. Exemplary NGS platforms using the CRT method include, but are not limited to, Illumina/Solexa Genome Analyzer (GA), which uses the clonally amplified template method coupled with the four-color CRT method detected by total internal reflection fluorescence (TIRF); and Helicos BioSciences/HeliScope, which uses the single-molecule template method coupled with the one-color CRT method detected by TIRF.

SBL uses DNA ligase and either one-base-encoded probes or two-base-encoded probes for sequencing. Typically, a fluorescently labeled probe is hybridized to its complementary sequence adjacent to the primed template. DNA ligase is used to ligate the dye-labeled probe to the primer. Fluorescence imaging is performed to determine the identity of the ligated probe after non-ligated probes are washed away. The fluorescent dye can be removed by using cleavable probes to regenerate a 5′-PO₄ group for subsequent ligation cycles. Alternatively, a new primer can be hybridized to the template after the old primer is removed. Exemplary SBL platforms include, but are not limited to, Life/APG/SOLiD (support oligonucleotide ligation detection), which uses two-base-encoded probes.

Pyrosequencing method is based on detecting the activity of DNA polymerase with another chemiluminescent enzyme. Typically, the method allows sequencing of a single strand of DNA by synthesizing the complementary strand along it, one base pair at a time, and detecting which base was actually added at each step. The template DNA is immobile, and solutions of A, C, G, and T nucleotides are sequentially added and removed from the reaction. Light is produced only when the nucleotide solution complements the first unpaired base of the template. The sequence of solutions which produce chemiluminescent signals allows the determination of the sequence of the template. Exemplary pyrosequencing platforms include, but are not limited to, Roche/454, which uses DNA templates prepared by emPCR with 1-2 million beads deposited into PTP wells.

Real-time sequencing involves imaging the continuous incorporation of dye-labeled nucleotides during DNA synthesis. Exemplary real-time sequencing platforms include, but are not limited to, Pacific Biosciences platform, which uses DNA polymerase molecules attached to the surface of individual zero-mode waveguide (ZMW) detectors to obtain sequence information when phospholinked nucleotides are being incorporated into the growing primer strand; Life/VisiGen platform, which uses an engineered DNA polymerase with an attached fluorescent dye to generate an enhanced signal after nucleotide incorporation by fluorescence resonance energy transfer (FRET); and LI-COR Biosciences platform, which uses dye-quencher nucleotides in the sequencing reaction.

Other sequencing methods for NGS include, but are not limited to, nanopore sequencing, sequencing by hybridization, nano-transistor array based sequencing, polony sequencing, scanning tunneling microscopy (STM) based sequencing, and nanowire-molecule sensor based sequencing.

Nanopore sequencing involves electrophoresis of nucleic acid molecules in solution through a nano-scale pore which provides a highly confined space within which single-nucleic acid polymers can be analyzed. Exemplary methods of nanopore sequencing are described, e.g., in Branton D. et al., Nat Biotechnol. 2008; 26(10):1146-53.

Sequencing by hybridization is a non-enzymatic method that uses a DNA microarray. Typically, a single pool of DNA is fluorescently labeled and hybridized to an array containing known sequences. Hybridization signals from a given spot on the array can identify the DNA sequence. The binding of one strand of DNA to its complementary strand in the DNA double-helix is sensitive to even single-base mismatches when the hybrid region is short or is specialized mismatch detection proteins are present. Exemplary methods of sequencing by hybridization are described, e.g., in Hanna G. J. et al., J. Clin. Microbiol. 2000; 38 (7): 2715-21; and Edwards J. R. et al., Mut. Res. 2005; 573 (1-2): 3-12.

Polony sequencing is based on polony amplification and sequencing-by-synthesis via multiple single-base-extensions (FISSEQ). Polony amplification is a method to amplify DNA in situ on a polyacrylamide film. Exemplary polony sequencing methods are described, e.g., in US Patent Application Publication No. 2007/0087362.

Nano-transistor array based devices, such as Carbon NanoTube Field Effect Transistor (CNTFET), can also be used for NGS. For example, DNA molecules are stretched and driven over nanotubes by micro-fabricated electrodes. DNA molecules sequentially come into contact with the carbon nanotube surface, and the difference in current flow from each base is produced due to charge transfer between the DNA molecule and the nanotubes. DNA is sequenced by recording these differences. Exemplary Nano-transistor array based sequencing methods are described, e.g., in U.S. Patent Application Publication No. 2006/0246497.

Scanning tunneling microscopy (STM) can also be used for NGS. STM uses a piezo-electric-controlled probe that performs a raster scan of a specimen to form images of its surface. STM can be used to image the physical properties of single DNA molecules, e.g., generating coherent electron tunneling imaging and spectroscopy by integrating scanning tunneling microscope with an actuator-driven flexible gap. Exemplary sequencing methods using STM are described, e.g., in U.S. Patent Application Publication No. 2007/0194225.

A molecular-analysis device which is comprised of a nanowire-molecule sensor can also be used for NGS. Such device can detect the interactions of the nitrogenous material disposed on the nanowires and nucleic acid molecules such as DNA. A molecule guide is configured for guiding a molecule near the molecule sensor, allowing an interaction and subsequent detection. Exemplary sequencing methods using nanowire-molecule sensor are described, e.g., in U.S. Patent Application Publication No. 2006/0275779.

Double ended sequencing methods can be used for NGS. Double ended sequencing uses blocked and unblocked primers to sequence both the sense and antisense strands of DNA. Typically, these methods include the steps of annealing an unblocked primer to a first strand of nucleic acid; annealing a second blocked primer to a second strand of nucleic acid; elongating the nucleic acid along the first strand with a polymerase; terminating the first sequencing primer; deblocking the second primer; and elongating the nucleic acid along the second strand. Exemplary double ended sequencing methods are described, e.g., in U.S. Pat. No. 7,244.567.

### Data Analysis

After NGS reads have been generated, they can be aligned to a known reference sequence or assembled de novo.

For example, identifying genetic variations such as single-nucleotide polymorphism and structural variants in a sample (e.g., a tumor sample) can be accomplished by aligning NGS reads to a reference sequence (e.g., a wild-type sequence). Methods of sequence alignment for NGS are described e.g., in Trapnell C, and Salzberg S. L. Nature Biotech., 2009, 27:455-457.

Examples of de novo assemblies are described, e.g., in Warren R. et al., Bioinformatics, 2007, 23:500-501; Butler J. et al., Genome Res., 2008, 18:810-820; and Zerbino D. R, and Bimey E., Genome Res., 2008, 18:821-829.

Sequence alignment or assembly can be performed using read data from one or more NGS platforms, e.g., mixing Roche/454 and Illumina/Solexa read data.

Algorithms and methods for data analysis are described in U.S. Ser. No. 61/428,568, filed Dec. 30, 2010, incorporated herein by reference.

### Fusion Expression Level

In certain embodiments, expression level of a fusion described herein can also be assayed. Fusion expression can be assessed by any of a wide variety of methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In certain embodiments, activity of a particular gene is characterized by a measure of gene transcript (e.g., mRNA), by a measure of the quantity of translated protein, or by a measure of gene product activity. Fusion expression can be monitored in a variety of ways, including by detecting mRNA levels, protein levels, or protein activity, any of which can be measured using standard techniques. Detection can involve quantification of the level of gene expression (e.g., genomic DNA, cDNA. mRNA, protein, or enzyme activity), or, alternatively, can be a qualitative assessment of the level of gene expression, in particular in comparison with a control level. The type of level being detected will be clear from the context.

Methods of detecting and/or quantifying the fusion gene transcript (mRNA or cDNA made therefrom) using nucleic acid hybridization techniques are known to those of skill in the art (see Sambrook et al. supra). For example, one method for evaluating the presence, absence, or quantity of cDNA involves a Southern transfer as described above. Briefly, the mRNA is isolated (e.g., using an acid guanidinium-phenol-chloroform extraction method, Sambrook et al. supra.) and reverse transcribed to produce cDNA. The cDNA is then optionally digested and run on a gel in buffer and transferred to membranes. Hybridization is then carried out using the nucleic acid probes specific for the cDNA of a fusion described herein, e.g., using the probes and primers described herein.

In other embodiments, expression of a fusion molecule described herein is assessed by preparing genomic DNA or mRNA/cDNA (i.e., a transcribed polynucleotide) from cells in a subject sample, and by hybridizing the genomic DNA or mRNA/cDNA with a reference polynucleotide which is a complement of a polynucleotide comprising the fusion, and fragments thereof. cDNA can, optionally, be amplified using any of a variety of polymerase chain reaction methods prior to hybridization with the reference polynucleotide. Expression of a fusion as described herein can likewise be detected using quantitative PCR (QPCR) to assess the level of expression.

### Detection of Fusion Polypeptide

The activity or level of a fusion polypeptide described herein can also be detected and/or quantified by detecting or quantifying the expressed polypeptide. The fusion polypeptide can be detected and quantified by any of a number of means known to those of skill in the art. These can include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, immunohistochemistry (IHC) and the like. A skilled artisan can adapt known protein/antibody detection methods.

Another agent for detecting a fusion polypeptide is an antibody molecule capable of binding to a polypeptide corresponding to a marker, e.g., an antibody with a detectable label. Techniques for generating antibodies are described herein. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

In another embodiment, the antibody is labeled, e.g., a radio-labeled, chromophore-labeled, fluorophore-labeled, or enzyme-labeled antibody. In another embodiment, an antibody derivative (e.g., an antibody conjugated with a substrate or with the protein or ligand of a protein-ligand pair {e.g., biotin-streptavidin}), or an antibody fragment (e.g., a single-chain antibody, an isolated antibody hypervariable domain, etc.) which binds specifically with a fusion protein described herein, is used.

Fusion polypeptides from cells can be isolated using techniques that are known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, New York).

Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another embodiment, Western blot (immunoblot) analysis is used to detect and quantify the presence of a polypeptide in the sample.

In another embodiment, the polypeptide is detected using an immunoassay. As used herein, an immunoassay is an assay that utilizes an antibody to specifically bind to the analyte. The immunoassay is thus characterized by detection of specific binding of a polypeptide to an anti-antibody as opposed to the use of other physical or chemical properties to isolate, target, and quantify the analyte.

The fusion polypeptide is detected and/or quantified using any of a number of immunological binding assays (see, e.g., U.S. Pat. Nos. 4,366,241; 4,376,110; 4,517,288; and 4,837,168). For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

### Kits

In one aspect, the invention features, a kit, e.g., containing an oligonucleotide having a mutation described herein, e.g., a fusion molecule described herein. Optionally, the kit can also contain an oligonucleotide that is the wild type counterpart of the mutant oligonucleotide.

A kit featured in the invention can include a carrier, e.g., a means being compartmentalized to receive in close confinement one or more container means. In one embodiment the container contains an oligonucleotide, e.g., a primer or probe as described above. The components of the kit are useful, for example, to diagnose or identify a mutation in a tumor sample in a patient. The probe or primer of the kit can be used in any sequencing or nucleotide detection assay known in the art, e.g., a sequencing assay, e.g., an NGS method, RT-PCR, or in situ hybridization.

In some embodiments, the components of the kit are useful, for example, to diagnose or identify a fusion described herein in a tumor sample in a patient, and to accordingly identify an appropriate therapeutic agent to treat the cancer.

A kit featured in the invention can include, e.g., assay positive and negative controls, nucleotides, enzymes (e.g., RNA or DNA polymerase or ligase), solvents or buffers, a stabilizer, a preservative, a secondary antibody, e.g., an anti-HRP antibody (IgG) and a detection reagent.

An oligonucleotide can be provided in any form, e.g., liquid, dried, semi-dried, or lyophilized, or in a form for storage in a frozen condition.

Typically, an oligonucleotide, and other components in a kit are provided in a form that is sterile. An oligonucleotide, e.g., an oligonucleotide that contains a mutation, e.g., a fusion described herein, or an oligonucleotide complementary to a fusion described herein, is provided in a liquid solution, the liquid solution generally is an aqueous solution, e.g., a sterile aqueous solution. When the oligonucleotide is provided as a dried form, reconstitution generally is accomplished by the addition of a suitable solvent. The solvent, e.g., sterile buffer, can optionally be provided in the kit.

The kit can include one or more containers for the composition containing an oligonucleotide in a concentration suitable for use in the assay or with instructions for dilution for use in the assay. In some embodiments, the kit contains separate containers, dividers or compartments for the oligonucleotide and assay components, and the informational material. For example, the oligonucleotides can be contained in a bottle or vial, and the informational material can be contained in a plastic sleeve or packet. In other embodiments, the separate elements of the kit are contained within a single, undivided container. For example, an oligonucleotide composition is contained in a bottle or vial that has attached thereto the informational material in the form of a label. In some embodiments, the kit includes a plurality (e.g., a pack) of individual containers, each containing one or more unit forms (e.g., for use with one assay) of an oligonucleotide. For example, the kit includes a plurality of ampoules, foil packets, or blister packs, each containing a single unit of oligonucleotide for use in sequencing or detecting a mutation in a tumor sample. The containers of the kits can be air tight and/or waterproof. The container can be labeled for use.

For antibody-based kits, the kit can include: (1) a first antibody (e.g., attached to a solid support) which binds to a fusion polypeptide; and, optionally, (2) a second, different antibody which binds to either the polypeptide or the first antibody and is conjugated to a detectable agent.

In one embodiment, the kit can include informational material for performing and interpreting the sequencing or diagnostic. In another embodiment, the kit can provide guidance as to where to report the results of the assay, e.g., to a treatment center or healthcare provider. The kit can include forms for reporting the results of a sequencing or diagnostic assay described herein, and address and contact information regarding where to send such forms or other related information; or a URL (Uniform Resource Locator) address for reporting the results in an online database or an online application (e.g., an app). In another embodiment, the informational material can include guidance regarding whether a patient should receive treatment with a particular chemotherapeutic drug, depending on the results of the assay.

The informational material of the kits is not limited in its form. In many cases, the informational material, e.g., instructions, is provided in printed matter, e.g., a printed text, drawings, and/or photographs, e.g., a label or printed sheet. However, the informational material can also be provided in other formats, such as computer readable material, video recording, or audio recording. In another embodiment, the informational material of the kit is contact information, e.g., a physical address, email address, website, or telephone number, where a user of the kit can obtain substantive information about the sequencing or diagnostic assay and/or its use in the methods described herein. The informational material can also be provided in any combination of formats.

In some embodiments, a biological sample is provided to an assay provider, e.g., a service provider (such as a third party facility) or a healthcare provider, who evaluates the sample in an assay and provides a read out. For example, in one embodiment, an assay provider receives a biological sample from a subject, such as a blood or tissue sample, e.g., a biopsy sample, and evaluates the sample using an assay described herein, e.g., a sequencing assay or in situ hybridization assay, and determines that the sample contains a fusion described herein. The assay provider, e.g., a service provider or healthcare provider, can then conclude that the subject is, or is not, a candidate for a particular drug or a particular cancer treatment regimen.

The assay provider can provide the results of the evaluation, and optionally, conclusions regarding one or more of diagnosis, prognosis, or appropriate therapy options to, for example, a healthcare provider, or patient, or an insurance company, in any suitable format, such as by mail or electronically, or through an online database. The information collected and provided by the assay provider can be stored in a database.

Incorporated by reference herein in their entirety are any polynucleotide and polypeptide sequences which reference an accession number correlating to an entry in a public database, such as those maintained by the COSMIC database, available on the worldwide web at sanger.ac.uk/genetics/CGP/cosmic/; and the Institute for Genomic Research (TIGR) on the world wide web at tigr.org and/or the National Center for Biotechnology Information (NCBI) on the world wide web at ncbi.nlm.nih.gov.

### EXAMPLES

### Example 1

### Kinase Fusion is a Common Feature in Spitz Neoplasms

Spitzoid neoplasms are a group of melanocytic tumors with distinctive histopathologic features. They include benign tumors (Spitz nevi), malignant tumors (spitzoid melanomas), and tumors with borderline histopathologic features and uncertain clinical outcome (atypical Spitz tumors). Their genetic underpinnings are poorly understood, and alterations in common melanoma-associated oncogenes are typically absent.

This invention discovers that spitzoid neoplasms harbor kinase fusions of ROS1 (17%), NTRK1 (16%), ALK (10%), BRAF (5%), and RET (3%) in a mutually exclusive pattern. The chimeric proteins are constitutively active, stimulate oncogenic signaling pathways, are tumorigenic, and are found in the entire biologic spectrum of spitzoid neoplasms, including 55% of Spitz nevi, 56% of atypical Spitz tumors, and 39% of spitzoid melanomas. Kinase inhibitors suppress the oncogenic signaling of the fusion proteins in vitro. In summary, kinase fusions account for the majority of oncogenic aberrations in spitzoid neoplasms, and may serve as therapeutic targets for metastatic spitzoid melanomas.

Melanocytic neoplasms comprise several tumor types that are characterized by distinct clinical, pathologic, and genetic features. The clinical course of melanocytic tumors may be indolent (benign nevi), aggressive (malignant melanomas), or intermediate (melanocytic tumors of uncertain malignant potential). In 1948, Sophie Spitz, a pathologist from Memorial Sloan-Kettering Cancer Center, coined the term ‘melanoma of childhood’ for a group of melanocytic skin tumors composed of spindled or epithelioid melanocytes that developed predominantly in children and adolescents (Spitz, S. Am J Pathol 24, 591-609 (1948)). It later became clear that these tumors could also arise later in life, and that the majority of these neoplasms behaved in an indolent fashion, which led to the introduction of the term ‘Spitz nevus’ to indicate their benign nature. Malignant tumors with spitzoid histologic features were termed ‘spitzoid melanomas’, and these tumors often showed aggressive clinical behavior with widespread metastasis, similar to conventional melanomas. Tumors with histologic features overlapping those of Spitz nevi and spitzoid melanoma have been termed ‘atypical Spitz tumors’. Atypical Spitz tumors have the capacity to metastasize, but this is usually limited to the regional lymph nodes, and has little effect on patient survival (Ludgate, M. W. et al. Cancer 115, 631-41 (2009); Murali, R. et al. Ann Surg Oncol 15, 302-9 (2008)).

Genetic alterations in the majority of spitzoid neoplasms are not known, and they lack mutations in melanoma-associated oncogenes such as NRAS, KIT, GNAQ or GNA11 (Flaherty, K. T. et al., Cancer 12, 349-61 (2012)). However, subsets of spitzoid neoplasms characterized by distinct histopathologic features show HRAS mutations (Bastian, B. C. et al., Am J Pathol 157, 967-72 (2000)), or BRAF mutations combined with bi-allelic BAP1 loss (Wiesner, T. et al. Nat Genet 43, 1018-21 (2011); Wiesner, T. et al., Am J Surg Pathol 36, 818-30 (2012)), suggesting that activation of kinase pathways plays an important role in the pathogenesis of these tumors. It is anticipated that identification of additional genetic events in kinase pathways will contribute to a better understanding of the pathogenesis of spitzoid neoplasms, and facilitate the development of effective targeted therapies for primary and metastasizing tumors, as illustrated by the success of small molecule kinase inhibitors in prolonging the lives of patients with a broad range of malignancies (Chapman, P. B. et al., N Engl J Med 364, 2507-16 (2011); Kwak, E. L. et al. N Engl J Med 363, 1693-703 (2010); Druker, B. J. et al., N Engl J Med 344, 1031-7 (2001)).

For diagnostic reasons, spitzoid neoplasms are usually formalin-fixed and paraffin-embedded (FFPE) in their entirety, and nucleic acids isolated from FFPE tissue are typically degraded and of suboptimal quality. In addition, the percentage of neoplastic cells in spitzoid neoplasms is frequently low, due to the presence of varying numbers of admixed non-neoplastic lymphocytes, fibroblasts, and keratinocytes. The suboptimal quality of extracted nucleic acids and the low proportion of neoplastic melanocytic cells makes the identification of genetic aberrations in spitzoid neoplasms challenging and therefore a high sequencing coverage is necessary. For these reasons, a massively parallel sequencing approach was chosen with high sequencing coverage for previously described cancer genes to investigate genetic aberrations in spitzoid neoplasms.

In a discovery cohort of 30 Spitz nevi and 8 atypical Spitz tumors, genomic alterations were investigated by targeted massively parallel sequencing. Table 2 summarizes the clinical, histopathologic and genetic data. The findings were further validated in an independent cohort of 102 spitzoid neoplasms using interphase FISH (Table 3 summarizes the clinical, histopathologic and genetic data).

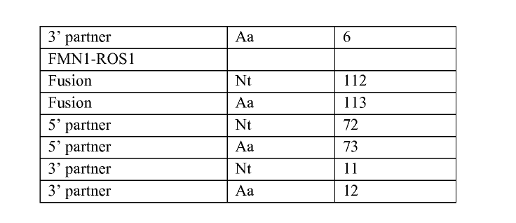

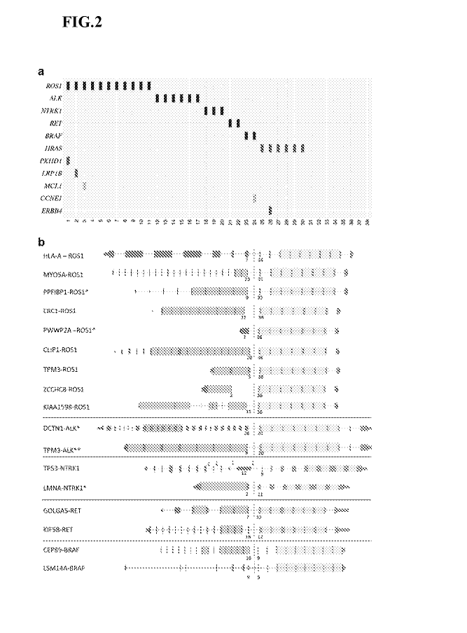

For targeted DNA sequencing, 3230 exons from 182 cancer-related genes and 37 introns of 14 genes commonly rearranged genes in cancer were sequenced. An average unique coverage of 997×, with 99.96% of exons being sequenced at ≧100× coverage. In addition, targeted transcriptome sequencing for 612 transcripts of kinases and kinase-related genes achieved an average of 56,588,548 unique read pairs per spitzoid neoplasm. Six tumors (16%) harbored HRAS c.182A>T (p.Q61L) mutations and exhibited histopathologic features characteristic of the previously-described HRAS-mutant Spitz nevus variant (Bastian, B. C. et al., Am J Pathol 157, 967-72 (2000)). Additionally, mutations in PKHD1, ERBB4, LRP1B, and amplifications of MCL1 and CCNE1 were identified in one case each (FIG. 2a). No sequence alterations were found in the known melanoma oncogenes BRAF, NRAS, KIT, GNAQ, or GNA11 or in other cancer-related genes listed in Table 4.

Kinase fusions were identified in 18 of 30 (60%) Spitz nevi and in 6 of 8 (75%) atypical Spitz tumors (FIGS. 2a and 2b). The rearrangements included gene fusions of the membrane-bound receptor tyrosine kinases ROS1 (n=11, 29%), ALK (n=6, 16%), NTRK1 (n=3, 8%), and RET (n=2, 5%). Two spitzoid neoplasms harbored t(7; 19(q34; q13) translocations that involved the serine/threonine kinase BRAF, but with different 5′-partners (CEP89 and LSM14A) in each tumor. RT-PCR with breakpoint flanking primers were used to verify that the fusion genes were expressed and produced in-frame transcripts. Interphase FISH with breakpoint-flanking probes was used to confirm the disruptions at the ROS1, ALK, NTRK1, RET, and BRAF loci.

An independent cohort of 102 spitzoid neoplasms was used to validate the foregoing findings using interphase FISH (Table 3 summarizes the clinical, histopathologic and genetic data). In total, 140 spitzoid neoplasms were analyzed leading to the identification of fusions in 41 of 75 (55%) Spitz nevi, in 18 of 32 (56%) atypical Spitz tumors, and in 13 of 33 (39%) spitzoid melanomas (Table 1). Expression of the chimeric ROS1, NTRK1, ALK, and RET proteins was confirmed using immunohistochemistry. Cases harboring translocations showed moderate to strong staining for the corresponding fusion kinase, which was not observed in the cases lacking gene rearrangements. Patients with translocation-positive spitzoid neoplasms were younger (median 21 years) than patients whose tumors did not harbor translocations (median 32.5 years; Mann Whitney test P<0.001).

The methodology for identifying the fusion proteins is described below. The excised skin lesions were fixed in 4% neutral buffered formalin, embedded in paraffin, processed using routine histologic methods and stained with hematoxylin-eosin. Specimens were collected over a time period of 6 months and the histopathologic diagnosis of Spitz nevus, atypical Spitz tumor, and spitzoid melanoma was confirmed by at least three dermatopathologists. Specimens with insufficient tissue amount or severely degraded nucleic acids were excluded. In total, specimens from 140 patients were analyzed and the clinical, histologic, and genetic characteristics are summarized in Table 2 and Table 3.

3230 exons from 182 cancer-related genes (Table 4) and 37 introns of 14 genes (Table 5) commonly rearranged genes in cancer were sequenced with average depth-of-coverage of greater than 500×. Prior to DNA extraction, FFPE samples from all cases were reviewed to confirm that the tissue was of sufficient size to generate a minimum of 50 ng of DNA and that this DNA would be derived from areas that contained a minimum of 20% melanocytic nuclei. DNA was isolated from 40 μm thick sections of formalin-fixed, paraffin-embedded (FFPE) tissue. DNA sequencing was performed on indexed, adaptor ligation, hybridization-captured libraries (Agilent SureSelect custom kit). Sequencing was performed on the HiSeq-2000 instrument (Illumina), with 49×49 paired reads to an average depth of 997× (Table 6).

Total RNA extracted from 40 μm thick sections of FFPE tumor was reverse-transcribed with random hexamer primers using the SuperScript® III First-Strand Synthesis System (Invitrogen). Double stranded cDNA was synthesized with the NEBNext® mRNA Second Strand Synthesis Module (New England Biolabs) (D'Alessio, J. M. & Gerard, G. F. Nucleic Acids Res 16, 1999-2014 (1988)). Hybrid selection of indexed, adaptor-ligated libraries was performed using the cDNA Kinome hybridization kit with 612 transcripts of kinases and kinase-related genes (Agilent SureSelect Human Kinome Kit) (Levin, J. Z. et al. Genome Biol 10, R115 (2009)). Selected libraries were sequenced on the HiSeq-2000 instrument (Illumina) with 49×49 paired reads. For RNA sequencing, a sequencing approach targeting 612 transcripts of kinases and kinase-related genes was used. A high number of unique read pairs (≧50,000,000) was sought per sample (Table 7).

Sequence data from gDNA and cDNA was mapped to the reference human genome (hg19) using the BWA aligner and processed using publically available SAMtools (Li, H. et al. Bioinformatics 25, 2078-9 (2009)), Picard (http://picard.sourceforge.net) and GATK (McKenna, A. et al. Genome Res 20, 1297-303 (2010)). Genomic base substitutions and indels were detected using custom tools optimized for mutation calling in heterogeneous tumor samples, based on detecting common sequence variations and local sequence assembly. Variations were filtered using dbSNP and a custom artifact database, then annotated for known and likely somatic mutations using COSMIC. Copy number alterations were detected by comparing targeted genomic DNA sequence coverage with a process-matched normal control sample. Genomic rearrangements were detected by clustering chimeric reads mapping to targeted introns/exons. Expression levels were determined by analyzing cDNA sequence coverage of targeted exons. Table 11 summarizes the predicted genomic coordinates of the fusion genes.

All gene fusions were validated by qRT-PCR from cDNA using the primers listed in Table 8 and analysis on a Bioanalyzer (Agilent). Specific PCR amplicons were only detected with the appropriate combination of primers and template, and not with negative controls. The nucleotide sequence at the fusion site was confirmed by Sanger sequencing.

These results show that kinase fusions are important mechanisms of oncogene activation in spitzoid neoplasms. In aggregate, 72 of 140 (51.4%) spitzoid neoplasms harbored fusions involving the receptor tyrosine kinases ROS1 (17%), NTRK1 (16%), ALK (10%), RET (3%), and the serine/threonine kinase BRAF (5%). All fusions occurred in a mutually exclusive pattern (FIG. 2a), and no fusions were detected in tumors with HRAS mutations. All of the sequenced kinase fusions created chimeric proteins that retained the intact kinase domain at the 3′ end of the fusion transcript (FIG. 2b), a pattern similar to that observed in other cancers that harbor rearrangements involving these kinases (Lipson, D. et al., Nat Med 18, 382-4 (2012); Soda, M. et al., Nature 448, 561-6 (2007); Takeuchi, K. et al., Nat Med 18, 378-81 (2012)). All recombination sites between kinases and translocation partners involved the canonical intronic recombination sites, similar to those described in other types of cancers (ROS1: intron 33-35; ALK: intron 19; NTRK1: intron 8-10; RET: intron 11; BRAF: intron 8) (Forbes, S. A. et al., Nucleic Acids Res 39, D945-50 (2011)). The majority of the novel 5′ fusion partners identified in our study, namely those of ROS1 (PWWP2A, PPFIBP1, ERC1, MYO5A, CLIP1, HLA-A, KIAA1598 and ZCCHC8), ALK (DCTN1), NTRK (LMNA), and BRAF (CEP89 and LSM14A) contributed coiled-coil domains to the fusion proteins. The coiled-coil domains may play similar roles to the established 5′ fusion partners (TPM3 (Lamant, L. et al., Blood 93, 3088-95 (1999)), GOLGA5 (Rabes, H. M. et al., Clin Cancer Res 6, 1093-103 (2000)), KIF1B(Lipson. D. et al., Nat Med 18, 382-4 (2012)) of these kinases in promoting dimerization and auto-activation of the kinases (Cohen, C. & Parry, D. A. Science 263, 488-9 (1994); McWhirter, J. R., Galasso, D. L. & Wang, J. Y. Mol Cell Biol 13, 7587-95 (1993)).

Rearrangements of the kinases ROS1, ALK, NTRK1, RET and BRAF with similar fusion sites have previously been described in various types of aggressive tumors. ROS1 fusions have been described in various cancer types, including lung carcinoma (Rikova, K. et al. Cell 131, 1190-203 (2007)), glioblastoma (Birchmeier, C., Sharma, S. & Wigler, M. Proc Natl Acad. Sci USA 84, 9270-4 (1987)), and cholangiocarcinoma (Gu, T. L. et al. PLoS One 6, e15640 (2011)). ALK fusions have been described in anaplastic large cell lymphoma (Lamant, L. et al., Blood 93, 3088-95 (1999), lung cancer (Soda, M. et al., Nature 448, 561-6 (2007)), inflammatory myofibroblastic tumors (Lawrence, B. et al. Am J Pathol 157, 377-84 (2000)) and anecdotal studies have also reported ALK fusions in acral melanoma (Niu, H. T. et al. Pigment Cell Melanoma Res (2013)). RET fusions, including the pericentric inversions giving rise to the KIF5B-RET fusion observed in spitzoid neoplasms, have been shown to drive lung cancer formation (Lipson, D. et al., Nat Med 18, 382-4 (2012); Takeuchi, K. et al., Nat Med 18, 378-81 (2012); Kohno, T. et al. Nat Med 18, 375-7 (2012)), and an interchromosomal GOLGA5-RET rearrangement was first described in papillary thyroid carcinomas occurring in children exposed to radioactive fallout from the Chernobyl nuclear accident (Rabes, H. M. et al. Clin Cancer Res 6, 1093-103 (2000)). BRAF fusions have been identified in pilocytic astrocytomas (Jones, D. T. et al. Cancer Res 68, 8673-7 (2008)), papillary thyroid carcinoma (Ciampi, R. et al. J Clin Invest 115, 94-101 (2005)), and rarely in melanocytic tumors (Palanisamy, N. et al. Nat Med 16, 793-8 (2010); Botton, T. et al. Pigment Cell Melanoma Res (2013)). In addition, these data that patients with translocation-positive spitzoid neoplasms were younger than patients whose tumors lacked translocations is similar to observations that lung cancers (Takeuchi, K. et al., Nat Med 18, 378-81 (2012)), thyroid cancers (Rabes. H. M. et al., Clin Cancer Res 6, 1093-103 (2000)), and astrocytomas (Jones, D. T. et al. Cancer Res 68, 8673-7 (2008)) harboring kinase fusions are more common in younger patients.

In summary, genomic analysis of 140 spitzoid neoplasms reveals gene rearrangements of the kinases ROS1 (17%; n=24), NTRK (16%; n=23), ALK (10%; n=14), BRAF (5%; n=7), and RET (3%; n=4) resulting in in-frame kinase fusions. These kinase fusions occur across the entire biologic spectrum of spitzoid neoplasms, including Spitz nevi (55%, fusions in 41 of 75 cases), atypical Spitz tumors (56%; fusions in 18 of 32 cases), and spitzoid melanomas (39%; fusions in 13 of 33 cases). The chimeric proteins appear in a mutually exclusive pattern, are constitutively active, stimulate oncogenic signaling pathways, are tumorigenic and may serve as diagnostic markers and as therapeutic targets for aggressive or metastasizing Spitz tumors.

TABLE 1
Frequency of kinase fusions in spitzoid neoplasms.

| | | Atypical | Spitzoid | |
|---|---|---|---|---|
| | Spitz nevus | Spitz tumor | melanoma | Total |
| | (n = 75) | (n = 32) | (n = 32) | (n= 140) |
| | % (number | % (number | % (number | % (number |
| Fusion | of cases) | of cases) | of cases) | of cases) |
| ROS1 | 25.3% (19) | 6.3% (2) | 9.1% (3) | 17.1% (24) |
| ALK | 10.7% (8) | 15.6% (5) | 3% (1) | 10% (14) |
| NTRK1 | 10.7% (8) | 25% (8) | 21.2% (7) | 16.4% (23) |
| BRAF | 5.3% (4) | 6.3% (2) | 3% (1) | 5% (7) |
| RET | 2.7% (2) | 3.1% (1) | 3% (1) | 2.9% (4) |
| Total | 54.7% (41) | 56.3% (18) | 39.4% (13) | 51.4% (72) |

TABLE 2
Clinical and histopathological characteristics of the analyzed Spitz tumors using targeted next generation sequencing.

| | | | | | | Translocation/ | RT- | | |
|---|---|---|---|---|---|---|---|---|---|
| # | Sex | Age | Localization | Diagnosis | Histopathologic features | Mutation | PCR | FISH | IHC |
| 1 | M | 9 | Back, | Pigmented | Symmetrical and well-circumscribed lesion | CLIP1- | + | + | + |
| | | | left | Spindle | composed of nests of spindled and epithelioid, | ROS1 | | | |
| | | | | Cell Nevus | pigmented melanocytes along the epidermal- | | | | |
| | | | | | dermal junction and in the epidermis. Multiple | | | | |
| | | | | | Kamino bodies. | | | | |
| 2 | F | 19 | Arm, | Spitz nevus, | Dome-shaped, well-circumscribed compound | PPFIBP1- | + | + | + |
| | | | lower left | compound | proliferation of large epithelioid melanocytes | ROS1 | | | |
| | | | | | with vesicular nuclei with irregular epidermal | | | | |
| | | | | | hyperplasia. Permeative lymphocytic infiltrate. | | | | |
| 3 | F | 47 | Buttock, | Spitz nevus, | Well circumscribed junctional proliferating of | TPM3- | + | + | + |
| | | | right | junctional | large spindled and epithelioid melanocytes, some | ROS1 | | | |
| | | | | | of which have pleomorphic nuclei; slight | | | | |
| | | | | | epidermal hyperplasia. | | | | |
| 4 | F | 39 | Leg, | Spitz nevus, | Well-circumscribed, symmetrical compound | ZCCHC8- | + | + | + |
| | | | lower left | compound | proliferation of large spindled and epithelioid | ROS1 | | | |
| | | | | | melanocytes with pleomorphic nuclei and | | | | |
| | | | | | prominent nucleoli arranged in large, partially | | | | |
| | | | | | confluent nests. Irregular epidermal hyperplasia. | | | | |
| 5 | F | 21 | Leg, | Pigmented | Well-circumscribed, symmetrical compound | MYO5A- | + | + | + |
| | | | upper left | Spindle | proliferation of large spindled and epithelioid, | ROS1 | | | |
| | | | | Cell Nevus | hyperpigmented melanocytes arranged along the | | | | |
| | | | | | dermo-epidermal junction. Irregular epidermal | | | | |
| | | | | | hyperplasia. Kamino bodies. | | | | |
| 6 | M | 21 | n.a. | Atypical | Dome-shaped, asymmetrical compound | PWWP2A- | + | + | + |
| | | | | Spitz tumor | proliferation of large epithelioid melanocytes | ROS1 | | | |
| | | | | | with marked nuclear pleomorphism, | | | | |
| | | | | | hyperchromasia, and prominent nuclei. Lack of | | | | |
| | | | | | maturation. Epidermal hyperplasia and fibrosis | | | | |
| | | | | | of the dermis. | | | | |
| 7 | F | 8 | Leg, | Spitz nevus, | Symmetrical and well circumscribed compound | PPFIBP1- | + | + | + |
| | | | left | compound | proliferation of large spindled and epithelioid | ROS1 | | | |
| | | | | | melanocytes with large and pleomorphic nuclei. | | | | |
| 8 | M | 12 | Arm, | Spitz nevus, | Kamino bodies are present. | HLA-A- | + | + | + |
| | | | lower left | compound | Symmetrical and well circumscribed compound | ROS1 | | | |
| | | | | | proliferation of large epithelioid melanocytes | | | | |
| | | | | | with prominent nucleoli. Marked papillated | | | | |
| | | | | | epidermal hyperplasia. | | | | |
| 9 | M | 12 | Leg, | Spitz nevus, | Asymmetrical compound proliferation of | ERC1- | + | + | + |
| | | | lower left | compound | medium sized spindled and epithelioid | ROS1 | | | |
| | | | | | melanocytes with scattered enlarged nuclei. | | | | |
| | | | | | Permeative lymphocytic infiltrate. | | | | |
| 10 | F | 1 | Cheek, | Spitz nevus, | Asymmetrieal compound proliferation of large | PWWP2A- | + | + | + |
| | | | right | compound | spindled and epithelioid melanocytes with large | ROS1 | | | |
| | | | | | pleomorphic nuclei and a permeative | | | | |
| | | | | | lymphocytic infiltrate. | | | | |
| 11 | F | 17 | Leg, | Spitz nevus, | Symmetrical and well circumscribed compound | KIAA1598- | n.d. | n.d. | n.d |
| | | | lower right | compound | proliferation of large epithelioid melanocytes | ROS1 | | | |
| | | | | | with large and pleomorphic nuclei. Epidermal | | | | |
| | | | | | hyperplasia. | | | | |
| 12 | M | 14 | Buttock, | Atypical | Symmetrical, exophytic, predominantly | TPM3- | + | + | + |
| | | | right | Spitz tumor | intradermal of spindled and epithelioid cells with | ALK | | | |
| | | | | | vesicular nuclei and prominent nucleoli. Irregular | | | | |
| | | | | | epidermal hyperplasia. | | | | |
| 13 | M | 2 | Arm, | Atypical | Well circumscribed melalanocytic tumor. In the | TPM3- | + | + | + |
| | | | lower left | Spitz tumor | dermis nests of large spindle-shaped cells with | ALK | | | |
| | | | | | vesicular nuclei and prominent nucleoli arranged | | | | |
| | | | | | in fascicles streaming downward into the dermis. | | | | |
| | | | | | Edematous papillary dermis and marked irregular | | | | |
| | | | | | epidermal hyperplasia. | | | | |
| 14 | F | 16 | Buttock, | Spitz nevus, | Symmetrical and well circumscribed compound | TPM3- | + | + | + |
| | | | left | compound | proliferation of large spindled and epithelioid | ALK | | | |
| | | | | | melanocytes with large and pleomorphic nuclei. | | | | |
| | | | | | Epidermal hyperplasia. | | | | |
| 15 | F | 14 | n.a. | Spitz nevus, | Re-excision of a desmoplastic Spitz nevus. | TPM3- | + | + | + |
| | | | | desmoplastic | Spindled and epithelioid melanocytes with | ALK | | | |
| | | | | | desmoplastic stroma. Few Melanophages. | | | | |
| 16 | M | 19 | Arm, | Atypical | Asymmetrical compound proliferation composed | DCTN1- | + | + | + |
| | | | upper left | Spitz tumor | of large confluent nests of spindled epithelioid | ALK | | | |
| | | | | | melanocytes with abundant cytoplasm and | | | | |
| | | | | | vesicular nuclei with prominent nucleoli; | | | | |
| | | | | | permeative lymphocytic infiltrate and irregular | | | | |
| | | | | | epidermal hyperplasia. | | | | |
| 17 | M | 9 | n.a. | Atypical | Exophytic, focally ulcerated compound | DCTN1- | + | + | + |
| | | | | Spitz tumor | proliferation of spindled and eptihelioid cells | ALK | | | |
| | | | | | arranged in elongated nests, streaming downward | | | | |
| | | | | | into the dermis. Permeative lymphocytic | | | | |
| | | | | | infiltrate and marked irregular epidermal | | | | |
| | | | | | hyperplasia. | | | | |
| 18 | F | 24 | Foot, | Spitz nevus, | Symmetrical and well circumscribed compound | TP53- | + | + | + |
| | | | left | compound | proliferation of spindled and epithelioid | NTRK1 | | | |
| | | | | | melanocytes melanocytic arranged in large nests. | | | | |
| | | | | | Marked lymphocytic infiltrate. | | | | |
| 19 | M | 2 | n.a. | Atypical | Exophytic, compound proliferation of small | LMNA- | + | n.d. | n.d. |
| | | | | Spitz tumor | epithelioid cells with pleomorphic nuclei | NTRK1 | | | |
| | | | | | arranged in large, confluent nests. Marked | | | | |
| | | | | | irregular epidermal hyperplasia with thin, | | | | |
| | | | | | elongated rete ridges. | | | | |
| 20 | F | 23 | Leg, | Spitz nevus, | Symmetrical, well circumscribed compound | LMNA- | + | + | + |
| | | | upper right | compound | proliferation of large epithelioid melanocytes | NTRK1 | | | |
| | | | | | with large and pleomorphic nuclei. Epidermal | | | | |
| | | | | | hyperplasia. Lymphocytic infiltrate. | | | | |
| 21 | M | 50 | n.a. | Pigmented | Symmetrical and well circumscribed compound | GOLGA5- | + | + | + |
| | | | | Spindle Cell | proliferation of spindled and epithelioid, | RET | | | |
| | | | | Nevus | pigmented melanocytes with slight epidermal | | | | |
| | | | | | hyperplasia. | | | | |
| 22 | M | 4 | n.a. | Compound | Well-circumscribed compound proliferation of | KIF5B- | + | + | + |
| | | | | Spitz nevus | large, spindled and epithelioid melanocytes with | RET | | | |
| | | | | | irregular epidermal hyperplasia. | | | | |
| 23 | F | 27 | Leg, | Pigmented | Symmetrical and well circumscribed compound | CEP89- | − | + | + |
| | | | lower right | Spindle Cell | proliferation of spindled and epithelioid, | BRAF | | | |
| | | | | Nevus | pigmented melanocytes with slight epidermal | | | | |
| | | | | | hyperplasia. Numerous melanophages in the | | | | |
| | | | | | papillary dermis. | | | | |
| 24 | F | 18 | Buttock, | Spitz nevus, | Predominantly intradermal proliferation of | LSM14A- | n.d. | + | + |
| | | | left | intradermal | spindled and epithelioid melanocytes with | BRAF | | | |
| | | | | | pleomorphic nuclei singly and in clusters | | | | |
| | | | | | between thickened collagen fibers. | | | | |
| 25 | M | 65 | Back, | Spitz nevus, | Predominantly intradermal proliferation of large | HRAS | n.d. | n.d. | n.d. |
| | | | left | desmoplastic | spindled and epithelioid melanocytes. | c.182A > T, | | | |
| | | | | | Pronounced desmoplastic stroma reaction. | p.Q61L | | | |
| 26 | M | 43 | retro- | Spitz nevus, | Symmetrical and well circumscribed | HRAS | n.d. | n.d. | n.d. |
| | | | auricular, | intradermal | predominantly intradermal proliferation of large | c.182A > T, | | | |
| | | | right | | spindled and epithelioid melanocytes with large | p.Q61L; | | | |
| | | | | | and pleomorphic nuclei. Desmoplastic stroma | ERBB4 | | | |
| | | | | | reaction. | c.1354G > A | | | |
| 27 | M | 25 | Chest | Spitz nevus, | Predominantly intradermal proliferation of | HRAS | n.d. | n.d. | n.d. |
| | | | | intradermal | spindled and epithelioid melanocytes with | c.182A > T, | | | |
| | | | | | pleomorphic nuclei with prominent nucleoli and | p.Q61L | | | |
| | | | | | abundant cytoplasm. Thickened collagen fibers. | | | | |
| 28 | M | 60 | Back, | Spitz nevus, | Hyperpigmented, epidermal hyperplasia. | HRAS | n.d. | n.d. | n.d. |
| | | | left | desmoplaslic | Pronounced desmoplastic stroma reaction with | c.182A > T, | | | |
| | | | | | epithelioid melanocytes and permeative | p.Q61L | | | |
| | | | | | lymphocytic infiltrate. | | | | |
| 29 | F | 44 | Shoulder, | Spitz nevus, | Symmetrical and superficial proliferation of | HRAS | n.d. | n.d. | n.d. |
| | | | right | desmoplastic | melanocytes with amphophilic cytoplasm and | c.182A > T, | | | |
| | | | | | vesicular nuclei with prominent nucleoli between | p.Q61L | | | |
| | | | | | thickened collagen fibers. | | | | |
| 30 | M | 7 | Cheek, | Atypical | Dome-shaped, symmetrical compound | HRAS | n.d. | n.d. | n.d. |
| | | | left | Spitz tumor | proliferation of spindled and epithelioid | c.182A > T, | | | |
| | | | | | melanocytes with pleomorphic nuclei. Irregular | p.Q61L | | | |
| | | | | | epidermal hyperplasia. | | | | |
| 31 | F | 18 | Leg, | Atypical | Polypoid compound proliferation of large | no mutation | n.d. | n.d. | n.d. |
| | | | upper left | Spitz tumor | epithelioid melanocytes with abundant cytoplasm | or fusion | | | |
| | | | | | and vesicular nuclei and numerous mitoses. | | | | |
| | | | | | Permeative lymphocytic infiltrate and marked | | | | |
| | | | | | irregular epidermal hyperplasia. | | | | |
| 32 | F | 24 | Buttock, | Pigmented | Symmetrical and well circumscribed compound | no mutation | n.d. | n.d. | n.d. |
| | | | right | Spindle Cell | proliferation of large epithelioid pigmented | or fusion | | | |
| | | | | Nevus | melanocytes with pleomorphic nuclei and | | | | |
| | | | | | vesicular chromatin and epidermal hyperplasia. | | | | |
| 33 | F | 39 | Leg, | Spitz nevus, | Well circumscribed and symmetrical melanocytic | no mutation | n.d. | n.d. | n.d. |
| | | | upper right | compound | lesion; at the epidermal-dermal junction and in | or fusion | | | |
| | | | | | the epidermis large nests composed of spindle- | | | | |
| | | | | | shaped melanocytes. | | | | |
| 34 | F | 15 | Leg, | Spitz nevus, | Exophytic compound proliferation of large | no mutation | n.d. | n.d. | n.d. |
| | | | lower right | compound | spindled and epithelioid melanocytes with | or fusion | | | |
| | | | | | pleomorphic, nuclei. Marked irregular epidermal | | | | |
| | | | | | hyperplasia and permeative lymphocytic | | | | |
| | | | | | infiltrate. | | | | |
| 35 | F | 32 | Arm, | Spitz nevus, | Symmetrical compound proliferation of spindled | no mutation | n.d. | n.d. | n.d. |
| | | | lower left | compound | and epithelioid melanocytes with epidermal | or fusion | | | |
| | | | | | hyperplasia; Kamino bodies. | | | | |
| 36 | F | 25 | Knee, | Spitz nevus, | Symmetrical and well circumscribed compound | no mutation | n.d. | n.d. | n.d. |
| | | | right | compound | proliferation of epithelioid melanocytes; | or fusion | | | |
| | | | | | epidermal hyperplasia and lymphocytic infiltrate. | | | | |
| 37 | F | 16 | Leg, | Spitz nevus, | Symmetrical compound proliferation of | no mutation | n.d. | n.d. | n.d. |
| | | | lower right | compound | pigmented spindled and epithelioid melanocytes | or fusion | | | |
| | | | | | with large nuclei. Irregular epidermal | | | | |
| | | | | | hyperplasia. | | | | |
| 38 | F | 32 | Leg, | Spitz nevus, | Well circumscribed and symmetrical junctional | no mutation | n.d. | n.d. | n.d. |
| | | | upper left | compound | proliferation of spindled and epithelioid | or fusion | | | |
| | | | | | melanocytes with irregular epidermal | | | | |
| | | | | | hyperplasia. Kamino bodies. | | | | |

TABLE 3
Clinical and histopathological characteristics of the analyzed Spitz tumors using FISH.

| # | Sex | Age | Localization | Diagnosis | FISH | IHC |
|---|---|---|---|---|---|---|
| 39 | M | 13 | Elbow, left | Spitz nevus, compound | FISH | + |
| 40 | F | 21 | Calf, right | Spitz nevus, compound | ROS1 | + |
| 41 | F | 31 | Upper arm, left | Spitz nevus, compound | PWWP2A-ROS1 | + |
| 42 | M | 6 | Knee, right | Spitz nevus, compound | PPFIBP1-ROS1 | + |
| | | | | pigmented | | |
| 43 | F | 30 | Foot, right | Spitz nevus, compound | ROS1 | + |
| | | | | pigmented | | |
| 44 | F | 39 | Back, left | Spitz nevus, compound | ROS1 | + |
| | | | | pigmented | | |
| 45 | F | 12 | Leg, right | Spitz nevus, desmoplastic | ROS1 | + |
| 46 | F | 29 | Arm, left | Spitz nevus, intraepidermal | ROS1 | + |
| 47 | F | 5 | Foot, right | Pigmented Spindle Cell Nevus | ROS1 | + |
| 48 | F | 55 | Gluteal, left | Atypical Spitz tumor | ROS1 | + |
| 49 | F | 29 | Thigh, right | Spitzoid melanoma | PWWP2A-ROS1 | + |
| 50 | F | 33 | Knee, right | Spitzoid melanoma | ROS1 | + |
| 51 | F | 59 | Leg, left | Spitzoid melanoma | ROS1 | + |
| 52 | F | 5 | Chin | Spitz nevus, compound | FMN1-ROS1 | + |
| 53 | F | 9 | Thigh, right lateral | Spitz nevus, compound | TPM3-ALK | + |
| 54 | M | 14 | Ear, right | Spitz nevus, compound | DCTN1-ALK | + |
| 55 | F | 28 | Back, right | Spitz nevus, compound | DCTN1-ALK | + |
| 56 | M | 35 | Leg, left | Spitz nevus, compound | DCTN1-ALK | + |
| 57 | M | 35 | Ear, left | Spitz nevus, compound | DCTN1-ALK | + |
| 58 | F | 17 | Ankle, left | Atypical Spitz tumor | TPM3-ALK | + |
| 59 | M | 17 | Leg, right lower | Spitzoid melanoma, 8 mm, | TPM3-ALK | + |
| | | | | ulcerated | | |
| 60 | F | 9 | Upper arm, right | Spitz nevus, compound | TPM3-ALK | + |
| 61 | F | 27 | Arm, upper left | Spitz nevus, compound | NTRK1 | + |
| 62 | F | 50 | Axilla, left | Spitz nevus, compound | NTRK1 | + |
| 63 | F | 5 | Buttock, right | Spitz tumor compound | NTRK1 | + |
| 64 | M | 2 | Hand, left | Spitz nevus, compound | NTRK1 | + |
| | | | | pigmented | | |
| 65 | F | 59 | Upper arm, right | Spitz nevus, compound | NTRK1 | + |
| | | | | pigmented | | |
| 66 | M | 18 | Leg, left | Atypical Spitz tumor | NTRK1 | + |
| 67 | F | 25 | Calf, left | Atypical Spitz tumor | NTRK1 | + |
| 68 | M | 28 | Back, right | Atypical Spitz tumor | LMNA-NTRK1 | + |
| 69 | M | 29 | Leg, right | Atypical Spitz tumor | NTRK1 | + |
| 70 | F | 18 | Trunk | Atypical Spitz tumor | NTRK1 | + |
| 71 | F | 21 | Arm, upper left | Atypical Spitz tumor | LMNA-NTRK1 | + |
| 72 | M | 21 | Calf, left | Atypical Spitz tumor | NTRK1 | + |
| 73 | M | 18 | Chest | Spitzoid melanoma, 9 mm | NTRK1 | + |
| 74 | M | 45 | Arm, left | Spitzoid melanoma, 1.2 mm | NTRK1 | + |
| 75 | F | 46 | Knee, right | Spitzoid melanoma, 2.3 mm | NTRK1 | + |
| 76 | F | 6 | Elbow, left | Spitzoid melanoma | NTRK1 | + |
| 77 | F | 39 | Knee, left | Spitzoid melanoma | NTRK1 | + |
| 78 | N | 73 | Shoulder, right | Spitzoid melanoma | NTRK1 | + |
| 79 | n.a. | n.a. | n.a. | Spitzoid melanoma | NTRK1 | + |
| 80 | M | 52 | Trunk | Atypical Spitz tumor | RET | + |
| 81 | F | 24 | Leg, right | Spitzoid melanoma, 0.75 mm | LMNA-RET | + |
| 82 | F | 15 | Forehead, right | Spitz nevus, compound | BRAF | + |
| 83 | F | 57 | Breast, right | Spitz nevus, desmoplastic | BRAF | + |
| 84 | F | 26 | Calf, right | Atypical Spitz tumor | BRAF | + |
| 85 | M | 32 | Chest, right | Atypical Spitz tumor | BRAF | + |
| 86 | F | 6 | Shoulder, right | Spitzoid melanoma, 5.4 mm | BRAF | + |
| 87 | M | 44 | Thigh, right | Spitz nevus, desmoplastic | BRAF | + |
| | | | | | amplification | |
| 88 | F | 11 | Periauricular, right | Atypical Spitz tumor | BRAF | + |
| | | | | | amplification | |
| 89 | F | 13 | Head | Atypical Spitz tumor | BAP1 loss | n.d. |
| 90 | F | 45 | Arm, upper right | Spitzoid melanoma | BAP1 loss | n.d. |
| 91 | F | 5 | Chin | Spitz nevus, compound | no fusion | n.d. |
| 92 | M | 14 | Thigh, right | Spitz tumor, compound | no fusion | n.d. |
| 93 | M | 15 | Knee, left | Spitz nevus, compound | no fusion | n.d. |
| 94 | M | 20 | Leg, right | Spitz nevus, compound | no fusion | n.d. |
| 95 | M | 22 | Shoulder, right | Spitz nevus, compound | no fusion | n.d. |
| 96 | F | 27 | Arm, upper right | Spitz nevus, compound | no fusion | n.d. |
| 97 | M | 28 | Neck | Spitz nevus, compound | no fusion | n.d. |
| 98 | F | 33 | Chest | Spitz nevus, compound | no fusion | n.d. |
| 99 | M | 38 | Back, right | Spitz nevus, compound | no fusion | n.d. |
| 100 | M | 46 | Breast, left | Spitz nevus, compound | no fusion | n.d. |
| 101 | F | 49 | Thigh, left | Spitz nevus, compound | no fusion | n.d. |
| 102 | M | 57 | Back, left | Spitz nevus, compound | no fusion | n.d. |
| 103 | F | 62 | Forearm, right | Spitz nevus, compound | no fusion | n.d. |
| 104 | F | 17 | Knee, left | Spitz nevus, compound | no fusion | n.d. |
| | | | | pigmented | | |
| 105 | F | 47 | Arm, right | Spitz nevus, desmoplastic | no fusion | n.d. |
| 106 | F | 19 | Leg, right | Spitz Nevus, intraepidermal | no fusion | n.d. |
| 107 | M | 51 | Hand, left dorsal | Spitz Nevus, intraepidermal | no fusion | n.d. |
| 108 | F | 7 | Leg, right | Pigmented Spindle Cell Nevus | no fusion | n.d. |
| 109 | M | 13 | Forehead, left | Pigmented Spindle Cell Nevus | no fusion | n.d. |
| 110 | F | 53 | Foot, left dorsal | Pigmented Spindle Cell Nevus | no fusion | n.d. |
| 111 | F | 59 | Back, left | Pigmented Spindle Cell Nevus | no fusion | n.d. |
| 112 | M | 8 | Ear, right | Atypical Spitz tumor | no fusion | n.d. |
| 113 | F | 12 | infraorbital, left | Atypical Spitz tumor | no fusion | n.d. |
| 114 | M | 12 | Thigh, left | Atypical Spitz tumor | no fusion | n.d. |
| 115 | M | 14 | Back, right | Atypical Spitz tumor | no fusion | n.d. |
| 116 | F | 18 | Gluteal, left | Atypical Spitz tumor | no fusion | n.d. |
| 117 | F | 22 | Knee, right | Atypical Spitz tumor | no fusion | n.d. |
| 118 | M | 31 | Calf, right | Atypical Spitz tumor | no fusion | n.d. |
| 119 | F | 32 | Breast, left | Atypical Spitz tumor | no fusion | n.d. |
| 120 | F | 38 | Thigh, left | Atypical Spitz tumor | no fusion | n.d. |
| 121 | M | 42 | Gluteal, right | Atypical Spitz tumor | no fusion | n.d. |
| 122 | F | 32 | Thigh, right | Spitzoid melanoma, 0.35 mm | no fusion | n.d. |
| 123 | F | 47 | Thigh, right | Spitzoid melanoma, 0.7 mm | no fusion | n.d. |
| 124 | F | 60 | Buttock, right | Spitzoid melanoma, 0.7 mm | no fusion | n.d. |
| 125 | M | 66 | Shoulder, left | Spitzoid melanoma, 0.8 mm | no fusion | n.d. |
| 126 | F | 72 | Knee, right | Spitzoid melanoma, 1.2 mm | no fusion | n.d. |
| 127 | M | 72 | Elbow, right | Spitzoid melanoma, 1.2 mm | no fusion | n.d. |
| 128 | F | 36 | Abdomen | Spitzoid melanoma, 3.3 mm | no fusion | n.d. |
| 129 | M | 4 | Arm, upper left | Spitzoid melanoma | no fusion | n.d. |
| 130 | F | 24 | Calf, right | Spitzoid melanoma | no fusion | n.d. |
| 131 | F | 27 | Thigh, left | Spitzoid melanoma | no fusion | n.d. |
| 132 | F | 33 | Arm, lower left | Spitzoid melanoma | no fusion | n.d. |
| 133 | M | 39 | Tight, left | Spitzoid melanoma | no fusion | n.d. |
| 134 | F | 40 | Arm, lower left | Spitzoid melanoma | no fusion | n.d. |
| 135 | F | 41 | Thigh, left | Spitzoid melanoma | no fusion | n.d. |
| 136 | M | 42 | Back, left | Spitzoid melanoma | no fusion | n.d. |
| 137 | M | 51 | Shoulder, left | Spitzoid melanoma | no fusion | n.d. |
| 138 | F | 64 | Thigh, right | Spitzoid melanoma | no fusion | n.d. |
| 139 | F | 65 | Arm, upper right | Spitzoid melanoma | no fusion | n.d. |
| 140 | F | 73 | Calf, left | Spitzoid melanoma | no fusion | n.d. |

TABLE 4
Genes sequenced across coding sequences.

| Gene | RefSeq | Gene | RefSeq | Gene | RefSeq | Gene | RefSeq |
|---|---|---|---|---|---|---|---|
| MTOR | NM_004958 | PIK3R1 | NM_181504 | SUFU | NM_016169 | ERBB2 | NM_001005862 |
| ARID1A | NM_006015 | APC | NM_001127511 | FGFR2 | NM_001144917 | RARA | NM_001145301 |
| MYCL1 | NM_005376 | PDGFRB | NM_002609 | HRAS | NM_005343 | STAT3 | NM_139276 |
| MPL | NM_005373 | NPM1 | NM_002520 | WT1 | NM_000378 | BRCA1 | NM_007298 |
| PTCH2 | NM_001166292 | FGFR4 | NM_022963 | MEN1 | NM_130801 | CD79B | NM_000626 |
| MUTYH | NM_001048171 | FLT4 | NM_002020 | CCND1 | NM_053056 | RPTOR | NM_020761 |
| CDKN2C | NM_001262 | FOXP4 | NM_001012426 | MRE11A | NM_005590 | CDH2 | NM_001792 |
| JUN | NM_002228 | CCND3 | NM_001136017 | GUCY1A2 | NM_000855 | SMAD2 | NM_001135937 |
| JAK1 | NM_002227 | PKHD1 | NM_138694 | ATM | NM_000051 | SMAD4 | NM_005359 |
| NRAS | NM_002524 | EPHA7 | NM_004440 | ETV6 | NM_001987 | CDH20 | NM_031891 |
| MCL1 | NM_001197320 | TNFAIP3 | NM_006290 | CBL | NM_005188 | BCL2 | NM_000633 |
| NTRK1 | NM_002529 | ESR1 | NM_001122742 | CHEK1 | NM_001274 | STK11 | NM_000455 |
| DDR2 | NM_006182 | IGF2R | NM_000876 | CCND2 | NM_001759 | DOT1L | NM_032482 |
| ABL2 | NM_007314 | CARD11 | NM_032415 | LRP6 | NM_002336 | GNA11 | NM_002067 |
| MDM4 | NM_002393 | HOXA3 | NM_153631 | KRAS | NM_033360 | MAP2K2 | NM_030662 |
| IKBKE | NM_014002 | INHBA | NM_002192 | ERBB3 | NM_001982 | INSR | NM_001079817 |
| AKT3 | NM_181690 | IKZF1 | NM_006060 | CDK4 | NM_000075 | SMARCA4 | NM_003072 |
| MYCN | NM_005378 | EGFR | NM_201284 | MDM2 | NM_002392 | JAK3 | NM_000215 |
| DNMT3A | NM_175629 | CDK6 | NM_001259 | PTPN11 | NM_002834 | CCNE1 | NM_001238 |
| ALK | NM_004304 | EPHB4 | NM_004444 | CDK8 | NM_001260 | CEBPA | NM_004364 |
| MSH2 | NM_000251 | PIK3CG | NM_002649 | FLT3 | NM_004119 | AKT2 | NM_001626 |
| MSH6 | NM_000179 | MET | NM_001127500 | FLT1 | NM_001160030 | CD79A | NM_001783 |
| LRP1B | NM_018557 | SMO | NM_005631 | BRCA2 | NM_000059 | ERCC2 | NM_000400 |
| IDH1 | NM_005896 | BRAF | NM_004333 | RB1 | NM_000321 | BCL2L1 | NM_138578 |
| ERBB4 | NM_005235 | EPHB6 | NM_004445 | 1RS2 | NM_003749 | SRC | NM_198291 |
| VHL | NM_000551 | EZH2 | NM_001203249 | BCL2L2 | NM_004050 | TOP1 | NM_003286 |
| RAF1 | NM_002880 | TNKS | NM_003747 | NKX2-1 | NM_001079668 | PLCG1 | NM_182811 |
| TGFBR2 | NM_003242 | GPR124 | NM_032777 | HSP90AA1 | NM_001017963 | AURKA | NM_198436 |
| MLH1 | NM_001167617 | FGFR1 | NM_001174065 | AKT1 | NM_001014431 | GNAS | NM_000516 |
| CTNNB1 | NM_001904 | PRKDC | NM_006904 | LTK | NM_206961 | ARFRP1 | NM_003224 |
| BAP1 | NM_004656 | MYC | NM_002467 | MAP2K1 | NM_002755 | RUNX1 | NM_001754 |
| MITF | NM_006722 | JAK2 | NM_004972 | SMAD3 | NM_005902 | ERG | NM_182918 |
| EPHA3 | NM_182644 | PTPRD | NM_130393 | BCL2A1 | NM_004049 | CRKL | NM_005207 |
| EPHA6 | NM_001080448 | CDKN2A | NM_001195132 | NTRK3 | NM_002530 | SMARCB1 | NM_003073 |
| EPHB1 | NM_004441 | CDKN2B | NM_004936 | IDH2 | NM_002168 | CHEK2 | NM_007194 |
| ATR | NM_001184 | PAX5 | NM_016734 | IGF1R | NM_000875 | NF2 | NM_181830 |
| PIK3CA | NM_006218 | GNAQ | NM_002072 | TSC2 | NM_001114382 | SOX10 | NM_006941 |
| SOX2 | NM_003106 | NTRK2 | NM_001018065 | CDH5 | NM_001795 | CRLF2 | NM_022148 |
| BCL6 | NM_001130845 | PTCH1 | NM_001083602 | CDH1 | NM_004360 | USP9X | NM_001039591 |
| FGFR3 | NM_022965 | ABL1 | NM_007313 | PHLPP2 | NM_015020 | KDM6A | NM_021140 |
| PDGFRA | NM_006206 | TSC1 | NM_001162427 | FANCA | NM_001018112 | ARAF | NM_001654 |
| KIT | NM_000222 | NOTCH1 | NM_017617 | TP53 | NM_001126114 | GATA1 | NM_002049 |
| KDR | NM_002253 | RET | NM_020630 | AURKB | NM_004217 | AR | NM_000044 |
| EPHA5 | NM_182472 | PTEN | NM_000314 | MAP2K4 | NM_003010 | TBX22 | NM_001109879 |
| TET2 | NM_001127208 | TNKS2 | NM_025235 | NF1 | NM_001042492 | PAK3 | NM_001128166 |
| FBXW7 | NM_033632 | RICTOR | NM_152756 | | | | |

TABLE 5
Introns included in hybridization selection assay.

| Gene | RefSeq | Intron |
|---|---|---|
| ALK | NM_004304 | 19 |
| RAF1 | NM_002880 | 5, 6, 7, 8, 9 |
| ETV5 | NM_004454 | 6, 7 |
| ETV1 | NM_001163151 | 3, 4 |
| EGFR | NM_201284 | 7 |
| BRAF | NM_004333 | 7, 8, 9, 10 |
| RET | NM_020630 | 9, 10, 11 |
| MLL | NM_005933 | 6, 7, 8, 9 |
| ETV6 | NM_001987 | 5, 6 |
| RARA | NM_001145301 | 2 |
| ETV4 | NM_001986 | 8 |
| TMPRSS2 | NM_005656 | 1, 2 |
| BCR | NM_004327 | 8, 13, 14 |
| EWSR1 | NM_013986 | 8, 9, 10, 11, 12, 13 |

TABLE 6
DNA sequencing statistics of 38 Spitz tumors.

| | | | | | | | | | | | |
|---|---|---|---|---|---|---|---|---|---|---|---|
| 1 | 43680902 | 99% | 0.42% | 0.66% | 51% | 15752607 | 188.91 | 0.82 | 1739.23 | 1212.99 | 1229 |
| 2 | 50758746 | 99% | 0.44% | 0.99% | 55% | 15942918 | 219.69 | 0.82 | 1636.51 | 1160.77 | 1178 |
| 3 | 48223361 | 99% | 0.44% | 0.54% | 86% | 4243484 | 174.85 | 0.84 | 1832.31 | 311.41 | 313 |
| 4 | 40855682 | 99% | 0.77% | 1.00% | 69% | 8641727 | 155.11 | 0.83 | 1883.42 | 700.47 | 716 |
| 5 | 49462206 | 99% | 0.68% | 1.01% | 59% | 13834857 | 186.85 | 0.83 | 1749.94 | 1058.57 | 1091 |
| 6 | 43492838 | 99% | 0.47% | 4.93% | 51% | 14443853 | 209.26 | 0.81 | 1663.85 | 1110.38 | 1088 |
| 7 | 42021747 | 99% | 0.44% | 1.34% | 51% | 14749854 | 202.86 | 0.83 | 1700.59 | 1121.88 | 1140 |
| 8 | 59626809 | 99% | 0.44% | 0.87% | 72% | 11491721 | 179.22 | 0.83 | 1786.83 | 868.61 | 900 |
| 9 | 44204047 | 99% | 0.55% | 3.17% | 55% | 13303731 | 193.44 | 0.81 | 1686.69 | 1013.32 | 1027 |
| 10 | 45574775 | 99% | 0.79% | 2.08% | 67% | 10253882 | 168.27 | 0.82 | 1814.29 | 813.2 | 831 |
| 11 | 15794873 | 99% | 0.36% | 0.44% | 21% | 8748665 | 160.73 | 0.83 | 1062.87 | 419.38 | 421 |
| 12 | 53716254 | 99% | 0.67% | 0.99% | 60% | 14718057 | 193.33 | 0.82 | 1712.72 | 1111.34 | 1134 |
| 13 | 39127695 | 99% | 0.77% | 1.90% | 50% | 13874632 | 183.07 | 0.82 | 1751.05 | 1095.29 | 1107 |
| 14 | 17206961 | 99% | 0.38% | 0.32% | 18% | 10433011 | 135.22 | 0.84 | 1140.84 | 532.05 | 544 |
| 15 | 18250776 | 99% | 0.38% | 0.33% | 17% | 10308621 | 144.82 | 0.8 | 1055.13 | 505.6 | 523 |
| 16 | 50629468 | 99% | 0.77% | 1.20% | 60% | 15219911 | 184.62 | 0.82 | 1743.42 | 1172.02 | 1195 |
| 17 | 49297848 | 92% | 0.58% | 2.85% | 53% | 16339804 | 191.39 | 0.82 | 1715.55 | 1268.24 | 1282 |
| 18 | 50497275 | 99% | 0.45% | 1.15% | 52% | 17125750 | 211.09 | 0.82 | 1662.58 | 1278.6 | 1303 |
| 19 | 52694441 | 92% | 0.57% | 2.45% | 51% | 18596366 | 190.34 | 0.83 | 1744.64 | 1462.92 | 1410 |
| 20 | 15232277 | 99% | 0.35% | 0.40% | 16% | 9270394 | 142.57 | 0.83 | 1108.45 | 465.12 | 470 |
| 21 | 69262651 | 99% | 0.68% | 0.51% | 81% | 8387727 | 164.58 | 0.84 | 1858.97 | 641.67 | 645 |
| 22 | 43007418 | 99% | 0.44% | 0.75% | 53% | 14342180 | 217.48 | 0.82 | 1632.36 | 1050.59 | 1067 |
| 23 | 56144067 | 99% | 0.78% | 1.46% | 67% | 12830210 | 169.64 | 0.83 | 1826.82 | 1020.46 | 1049 |
| 24 | 17637624 | 99% | 0.38% | 0.39% | 13% | 11095731 | 145.73 | 0.83 | 1103.36 | 561.21 | 565 |
| 25 | 17564962 | 99% | 0.37% | 0.38% | 16% | 10731098 | 137.25 | 0.83 | 1120.34 | 540.74 | 558 |
| 26 | 15227480 | 99% | 0.34% | 0.35% | 19% | 8960940 | 138.51 | 0.84 | 1124.25 | 441.06 | 457 |
| 27 | 18736706 | 99% | 0.37% | 0.45% | 22% | 10526919 | 132 | 0.83 | 1134.18 | 544.3 | 563 |
| 28 | 15656437 | 99% | 0.34% | 0.37% | 21% | 8844369 | 147.38 | 0.83 | 1096.61 | 429.15 | 445 |
| 29 | 17603229 | 99% | 0.35% | 0.32% | 23% | 9932176 | 138.01 | 0.84 | 1137.51 | 498.44 | 509 |
| 30 | 43078914 | 99% | 0.67% | 1.11% | 52% | 14286316 | 193.95 | 0.82 | 1696.45 | 1083.13 | 1097 |
| 31 | 34473544 | 99% | 0.47% | 3.05% | 67% | 8027432 | 150.9 | 0.82 | 1881.74 | 665.35 | 684 |
| 32 | 57040666 | 99% | 0.68% | 1.18% | 64% | 14054691 | 194 | 0.83 | 1725.66 | 1054.59 | 1081 |
| 33 | 53273598 | 99% | 0.69% | 1.59% | 65% | 12594967 | 163.05 | 0.83 | 1847.34 | 1010.5 | 1039 |
| 34 | 48792099 | 99% | 0.52% | 1.49% | 67% | 11330130 | 172.29 | 0.83 | 1811.07 | 890.55 | 915 |
| 35 | 56907738 | 99% | 0.69% | 1.45% | 58% | 16086659 | 215.45 | 0.81 | 1620.41 | 1164.01 | 1187 |
| 36 | 38757620 | 99% | 0.52% | 1.50% | 63% | 9936474 | 201.67 | 0.82 | 1696.5 | 740.84 | 757 |
| 37 | 49441431 | 99% | 0.80% | 1.47% | 74% | 8535510 | 160.81 | 0.83 | 1862.15 | 676.26 | 692 |
| 38 | 38597652 | 99% | 0.78% | 1.43% | 64% | 9526024 | 186.63 | 0.83 | 1766.35 | 734.23 | 752 |

TABLE 7
RNA sequencing statistics of 38 Spitz tumors.

| | | | | | | | | | |
|---|---|---|---|---|---|---|---|---|---|
| 1 | 52393754 | 86% | 0.67% | 3.44% | 69% | 97% | 51% | 5% | 267.73 |
| 2 | 42796746 | 87% | 0.63% | 4.17% | 63% | 97% | 52% | 5% | 259.49 |
| 3 | 122134074 | 86% | 0.73% | 2.47% | 90% | 97% | 49% | 5% | 199.9 |
| 4 | 46284520 | 85% | 0.89% | 2.50% | 76% | 97% | 46% | 7% | 168.96 |
| 5 | 50854826 | 85% | 0.94% | 3.45% | 70% | 97% | 50% | 6% | 243.83 |
| 6 | 66498690 | 88% | 0.69% | 2.76% | 68% | 97% | 53% | 5% | 363.71 |
| 7 | 52843870 | 87% | 0.66% | 3.47% | 72% | 97% | 50% | 5% | 240.26 |
| 8 | 50361907 | 86% | 0.77% | 3.15% | 72% | 97% | 48% | 6% | 224.35 |
| 9 | 57350489 | 85% | 0.73% | 3.00% | 69% | 96% | 52% | 4% | 304.8 |
| 10 | 61391661 | 86% | 0.77% | 3.20% | 76% | 97% | 49% | 5% | 243.61 |
| 11 | 46415279 | 77% | 0.46% | 2.84% | 73% | 98% | 47% | 2% | 655.67 |
| 12 | 61195040 | 86% | 0.94% | 3.31% | 68% | 97% | 50% | 4% | 320.23 |
| 13 | 49592222 | 86% | 0.73% | 2.79% | 71% | 97% | 49% | 7% | 246.93 |
| 14 | 39324605 | 82% | 0.50% | 2.69% | 63% | 98% | 46% | 2% | 511.44 |
| 15 | 52044254 | 82% | 0.37% | 2.29% | 72% | 98% | 47% | 2% | 722.76 |
| 16 | 56239578 | 86% | 0.95% | 3.72% | 68% | 96% | 51% | 5% | 300.2 |
| 17 | 57483583 | 87% | 0.70% | 2.40% | 74% | 97% | 49% | 9% | 242.05 |
| 18 | 54430378 | 87% | 0.65% | 2.75% | 69% | 97% | 50% | 5% | 267.22 |
| 19 | 62390150 | 87% | 0.73% | 3.27% | 70% | 97% | 48% | 7% | 304.07 |
| 20 | 45879456 | 79% | 0.47% | 2.57% | 74% | 98% | 45% | 2% | 631.49 |
| 21 | 53435566 | 85% | 1.06% | 2.76% | 77% | 97% | 47% | 5% | 189.36 |
| 22 | 48326968 | 87% | 0.65% | 3.20% | 70% | 97% | 49% | 5% | 227.8 |
| 23 | 43739879 | 86% | 0.82% | 3.64% | 73% | 97% | 51% | 6% | 195.2 |
| 24 | 40299110 | 82% | 0.45% | 2.32% | 66% | 98% | 50% | 2% | 489.09 |
| 25 | 52181406 | 81% | 0.45% | 1.37% | 81% | 98% | 48% | 1% | 638.24 |
| 26 | 38472733 | 80% | 0.45% | 2.19% | 64% | 98% | 48% | 2% | 490.99 |
| 27 | 36232684 | 80% | 0.55% | 2.15% | 66% | 98% | 51% | 2% | 422.81 |
| 28 | 50973629 | 78% | 0.43% | 1.83% | 76% | 98% | 47% | 1% | 677.15 |
| 29 | 44136276 | 79% | 0.50% | 1.84% | 80% | 98% | 45% | 2% | 614.67 |
| 30 | 50107832 | 88% | 0.88% | 3.29% | 64% | 96% | 51% | 5% | 308.49 |
| 31 | 85185779 | 83% | 0.96% | 2.74% | 82% | 97% | 45% | 5% | 231.76 |
| 32 | 60880957 | 86% | 0.94% | 2.86% | 74% | 97% | 49% | 5% | 255.47 |
| 33 | 50453581 | 84% | 1.13% | 3.09% | 72% | 97% | 46% | 5% | 211.36 |
| 34 | 54579307 | 83% | 0.77% | 3.08% | 77% | 96% | 49% | 7% | 201.21 |
| 35 | 51997530 | 87% | 0.89% | 3.69% | 63% | 97% | 51% | 5% | 312.72 |
| 36 | 56688409 | 86% | 0.70% | 3.05% | 75% | 97% | 52% | 5% | 239.64 |
| 37 | 41991585 | 84% | 0.82% | 3.30% | 68% | 97% | 51% | 6% | 221.39 |
| 38 | 42850478 | 85% | 0.74% | 2.87% | 70% | 97% | 49% | 6% | 210.31 |

TABLE 8
Primers used for RT-PCR.

| | Forward | Reverse |
|---|---|---|
| Fusion | Primer | Primer |
| CLIP1-ROS1 | 5′-GCAGGGACG | 5′-ATCTCCTCT |
| | AAGTCACAAGT | TGGGTTGGA |
| PPFIBP1-ROS1 | 5′-TGGTTTGCA | 5′-AGCACTGTC |
| | AGATGAAAGGA | ACCCCTTCC |
| TPM3-ROS1 | 5′-CCTGCAAAA | 5′-GGGAAGGCA |
| | GCTGGAAGAAG | GGAAGATTT |
| ZCCHC8-ROS1 | 5′-ACGAGGAGG | 5′-ATCTCCTCT |
| | ACGAAAATGG | TGGGTTGGA |
| MYO5A-ROS1 | 5′-GCAAGAAAA | 5′-AGCACTGTC |
| | AGAAGCCCTCA | ACCCCTTCC |
| PWWP2A-ROS1 | 5′-CTTCTCCGG | 5′-GCAAGAGAC |
| | GGTCCTCAT | GCAGAGTCA |
| HLA-A-ROS1 | 5′-GTGACAGTG | 5′-AAAGGTCAG |
| | CCCAGGGCTCTG | TGGGATTGT |
| ERC1-ROS1 | 5′-TGAAGGAGG | 5′-ATCTCCTCT |
| | TGGAAAATGAGA | TGGGTTGGA |
| DCTN1-ALK | 5′-CCTCCTTCA | 5′-GAGCTTGCT |
| | GGCATTGCTAC | CAGCTTGTA |
| TPM3-ALK | 5′-AGCCAAGCT | 5′-GCTTGCTCA |
| | GGAAAAGACAA | GCTTGTACTCA |
| GOLGA5-RET | 5′-AGAAGCTGA | 5′-CAAATTCGC |
| | TGGGCCAGATA | CTTCTCCTA |
| KIF5B-RET | 5′-TTAGCAGCA | 5′-CAAATTCGC |
| | TGTCAGCTTCG | CTTCTCCTA |
| TP53-NTRK1 | 5′-CCAGCCAAA | 5′-AGACCCCAA |
| | GAAGAAACCAC | AAGGTGTT |
| LMNA-NTRK1 | 5′-GAGGGCGAG | 5′-AGGAGCAGC |
| | CTGCATGAT | GTAGAAAGG |
| CEP89-BRAF | 5′-TAATGGCAG | 5′-CTCCATCAC |
| | AGTGCCTCAAA | CACGAAATCCT |

### Example 2

### Functional Studies of ROS1, ALK, NTRK1, RET and BRAF Fusions

After sequencing data revealed various kinase fusion products in FIGS. 1A, 1B and 1C, validation studies as well as functional studies were performed to investigate the role of these kinase fusions both in vitro and in vivo.

To validate sequencing data, interphase fluorescence in situ hybridization (FISH) was performed. A commercially available break-apart probe was used for ALK according to the manufacturer's protocol (Abbott, Des Plaines, Ill.). For ROS1, RET, NTRK1, and BRAF, break-apart fluorescence in situ hybridization (FISH) probes were prepared from BAC clones using standard procedures and labeled by nick translation with SpectrumGreen-dUTP and SpectrumnRed-dUTP (Abbott, Des Plaines, Ill.). The following flanking BAC clones were used: RET (red: RP11-379D20, RP11-124011; green: RP11-718J13, RP11-54P13), NTRK1 (red: RP11-284F21, RP11-66D17; green: RP11-1038N13, RP11-1059C21), ROS1 (red: RP11-379F24, RP11-103F10; green: RP11-1059G13, RP11-721K11), and BRAF (red: RP11-715H9, RP11-133N19; green: RP11-759K14, RP11-78806). The probes were hybridized on 5 μm-thick tissue sections, and the number and localization of the hybridization signals was assessed in a minimum of 100 interphase nuclei with well-delineated contours and at least 50% of neoplastic cells had to show a split signal to report a rearrangement of a kinase.

Immunohistochemical analysis (IHC) was performed on archival FFPE tumor specimens to confirm the expression of the fusion kinases and the activation of oncogenic pathways. A Discovery Ultra instrument with a multimer/DAB detection system (Ventana Medical Systems, Inc., Tucson, Ariz.) was used with appropriate negative and positive controls the following antibodies: ALK (clone D5F3; Cell Signaling, Danvers, Mass.; Dilution: 1:250), ROS1 (clone D4D6; Cell Signaling, Danvers, Mass.; Dilution: 1:25). NTRK1 (clone EP 058Y; Epitomics, Burlingame, Calif.; Dilution: 1:100), RET (clone EPR287; Epitomics, Burlingame, Calif.; Dilution: 1:250). The percentage of tumor cells exhibiting staining was scored by at least two independent pathologists.

The functional roles and the activation of oncogenic signaling pathways, were cloned PWWP2A-ROS1, DCTN1-ALK, LMNA-NTRK1, and GOLGA5-RET fusions and expressed them in melan-a cells (immortalized, non-tumorigenic mouse melanocytes). Expression vectors with green-fluorescent-protein (GFP) and the full-length, wild-type kinases ROS1, ALK, NTRK1, and RET were used as controls. Plasmids containing cDNAs of the genes of interest were obtained (Table 9) and their sequences were verified (Scaffidi & Misteli, Nat Cell Biol 10, 452-9 (2008); Johannessen, C. M. et al. Nature 468, 968-72 (2010); Yano, H. et al. J Neurosci Res 59, 356-64 (2000)). Non-synonymous mutations deviating from the RefSeq sequence found in the NTRK1 and RET plasmids were corrected using QuikChange site-directed mutagenesis (Agilent Technologies). Fusion constructs were generated by overlap extension. (Heckman, K. L. & Pease, L. R. Nat Protoc 2, 924-32 (2007)). Once PCR products containing the target cDNAs were generated, they were cloned into a pENTR vector using the pENTRD-TOPO cloning kit (Life Technologies). All constructs were subsequently cloned into the pLenti6.3/TO/V5-Dest backbone (Life Technologies) and checked by digestion and sequencing. The larger fusion genes involving ALK and ROS1 were also cloned into the PBX2.1 backbone of the PiggyBac Transposon system. (Choi, H. J. et al. PLoS One 8, e56949 (2013))

Melan-a cells were a gift; they were maintained in glutamine-containing RPMI-1640 supplemented with 10% heat-inactivated fetal bovine serum, 200 nM of 12-O-tetradecanoylphorbol-13-acetate (TPA), penicillin (100 units/mL) and streptomycin (50 mg/mL). 293FT cells were purchased from Life Technologies and maintained in DME-H21 medium containing 10% heat inactivated fetal bovine serum, MEM Non-Essential Amino Acids (0.1 mM), sodium pyruvate (1 mM), penicillin (100 units/mL) and streptomycin (50 mg/mL).

Lentiviruses were produced by transfecting 293FT cells with plasmid DNA in a pLenti6.3/TO/V5-Dest backbone together with ViraPower™ (Life Technologies) according to the manufacturer's protocol. Infections of melan-a cells were conducted in the presence of 10 μg/ml of Polybrene (Santa Cruz Biotechnology). Stably transduced melan-a cells expressing GOLGA5-RET, LMNA-NTRK1, PWWP2A-ROS1, RET, and NTRK1 were generated by infection with the respective lentivirus. ALK and ROS1 expressing melan-a cell lines were generated by cotransfection of the PBX2.1 construct together with pCMV-HyPBase transposase vector. Melan-a cell lines stably expressing GFP were generated using pLenti6.2/V5 and PBX2.1 vectors and used as a control. Cells were selected for at least 20 days using 5 μg/ml of blasticidin S-hydrochloride (Life Technologies) after transduction.

Anti-RET (#3220), anti-phospho-RET (Tyr905) (#3221), anti-ALK (#3791), anti-phospho-ALK (Tyr1096) (#6962), anti-ROS1 (#3266), anti-phospho ROS1 (Tyr2274) (#3078), anti-phospho-p44/42 MAPK (Erk1/2) (Thr202/Tyr204) (#9101), anti-AKT (#9272), anti-phospho-AKT (Ser473) (#9271), anti-S6 Ribosomal Protein (#2317), anti-phospho-S6 Ribosomal Protein (Ser235/236) (#2211), anti-p-PLCγ1 (#2822), anti-phospho-PLCγ1 (Tyr783) (#2821), anti-SHP2 (D50F2) (#3397), anti-phospho-SHP2 (Tyr 542) (#3751), as well as the secondary antibodies anti-rabbit IgG-HRP (#7076) and anti-mouse IgG-HRP (#7074) were purchased from Cell Signaling Technology (Danvers, Mass.). Anti-HSP60 (sc-1722) and anti-ERK2 (C-14) (sc-154) as well as the secondary antibodies anti-goat IgG-HRP (sc-2033) were purchased from Santa Cruz Biotechnology (Santa Cruz, Calif.). Anti-TrkA (ab37837) and anti-phospho-TrkA (Tyr408) (ab1445) were purchased from Abcam (Cambridge, Mass.).

Cell lysates were prepared in RIPA buffer supplemented with Halt protease and phosphatase inhibitor cocktail (Thermo Scientific). Equal amounts of protein, as measured by BCA protein assay, were resolved in 4-12% Bis-Tris NuPage gradient gels (Life Technologies) and transferred electrophoretically onto a PVDF 0.45-micron membrane. Membranes were blocked for 1 hour at room temperature in 5% BSA or non-fat milk in TBST before being incubated overnight at 4° C. with the primary antibodies. All primary antibodies were diluted 1:1000 in 5% BSA or non-fat milk in TBST. After 3 washes of 5 minutes in TBST, secondary antibodies were diluted 1:3000 in 5% non-fat milk in TBST and incubated for 1 hour at room temperature. After another 3 washes in TBST, detection of the signal was achieved by incubating the membrane on an ECL solution from Millipore and exposure on autoradiography films from Denville Scientific (Metuchen, N. J.). Vandetanib and cabozantinib were purchased from Selleckchem (Houston, Tex.). Crizotinib was purchased from Chemie Tek (Indianapolis, Ind.). AZ-23 was purchased from Axon Medchem (Groningen, The Netherlands).

ROS1 Fusions:

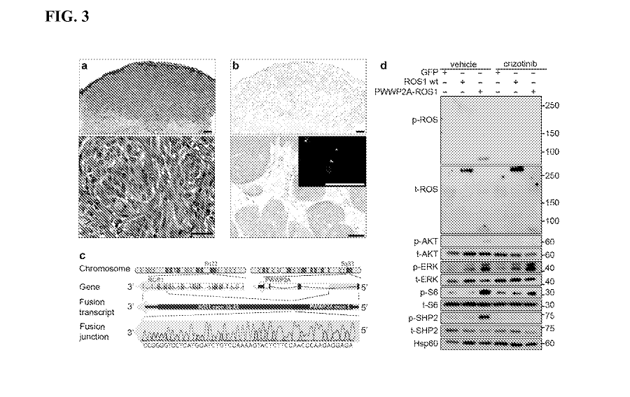

ROS1 rearrangements were found in 19 of 75 (25%) Spitz nevi, 2 of 32 (6%) atypical Spitz tumors, and 3 of 33 (9%) spitzoid melanoma (FIG. 3a). The ROS1 rearrangements fused the intact tyrosine kinase coding sequence of ROS1 to the 5′ portion of nine different partners (FIG. 3a). The expression of the chimeric ROS1 protein by immunohistochemistry was observed exclusively in cases with ROS1 rearrangements (FIGS. 3b and 3c). Expression of the PWWP2A-ROS1 fusion construct in melan-a cells showed increased phosphorylation of the fusion protein, suggesting that the chimeric protein is constitutively active. Consistent with this, the MAPK and PI3K pathways were strongly activated compared to the GFP and the wild-type, full-length ROS1 controls. The phosphorylation of PWWP2A-ROS1, AKT, S6 and SHP2, but not ERK, was at least partially inhibited by crizotinib, an FDA-approved drug for lung cancer with ALK translocations that acts as an ALK (Kwak, E. L. et al. N Engl J Med 363, 1693-703 (2010)) and ROS1 (Bergethon, K. et al. J Clin Oncol 30, 863-70 (2012)) inhibitor (FIG. 3d).

ALK Fusions:

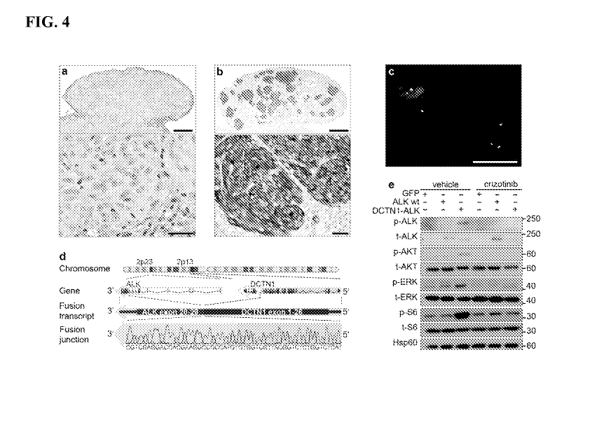

ALK fusions in 8 of 75 (10.7%) Spitz nevi, 5 of 32 (15.6%) atypical Spitz tumors, and 1 of 33 (3%) spitzoid melanoma (FIG. 4a). Expression of the chimeric ALK protein was confirmed by immunohistochemistry and the rearrangements were validated in all cases with ALK fusions with FISH (FIGS. 4h and 4c). Cases without ALK rearrangements by sequencing did not show ALK expression in immunohistochemistry or separated signals by FISH. TPM3 and DCTN1 were 5′ fusion partners for ALK rearrangements (FIG. 2b). TPM3-ALK fusions were detected in 8 spitzoid neoplasms. DCTN1 represents a novel fusion partner of ALK; it was found in 6 cases, and resulted from a balanced translocation between homologous copies of chromosome 2 (FIG. 4d). Expression of the DCTN1-ALK fusion construct in melan-a cells showed increased phosphorylation of the fusion protein compared to the wild-type, full-length ALK, suggesting that the chimeric protein is constitutively active. Similar to the ROS1 fusions, AKT, ERK, and S6 were more phosphorylated in melan-a cells with the ALK fusion construct compared to the GFP control. Crizotinib inhibited the ALK fusion-induced activation of the oncogenic PI3K and MAPK signaling pathways (FIG. 4e).

NTRK1 Fusions:

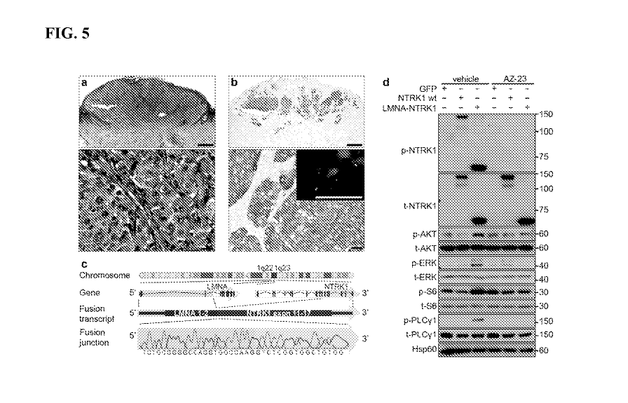

NTRK1 rearrangements were detected in 8 of 75 (10.7%) Spitz nevi, 8 of 32 (25%) atypical Spitz tumors, and 7 of 33 (21.2%) spitzoid melanoma (FIG. 5a). The expression of NTRK1 was confirmed with immunohistochemistry in cases with NTRK1 fusions (FIG. 5b), but was absent in cases without NTRK1 rearrangements. The LMNA-NTRK1 fusions involved the novel fusion partner LMNA (localized at 1q22), and were caused by a 743 kb deletion of chromosome 1 q, joining the first two exons of LMNA with NTRK1 starting at exon 11 (FIG. 5c). The interchromosomal TP53-NTRK1 translocation resulted from a fusion joining NTRK1 starting from exon 9, containing the tyrosine kinase domain, to the 3′UTR of TP53 (FIG. 2B). The expression of the LMNA-NTRK1 fusion construct in melan-a cells showed increased levels of phosphorylation of the fusion protein, AKT, ERK, S6 and PLCγ1 compared to the control cells. The strong activation of MAPK, PLCγ1 and PI3K pathways was inhibited by the NTRK1 inhibitor AZ-23 (FIG. 5d).

RET Fusion:

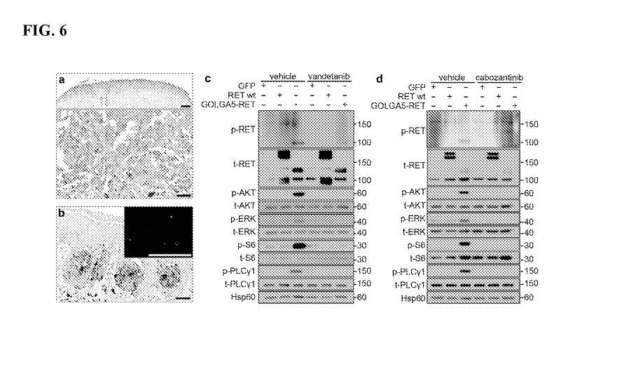

RET fusions in 2 of 75 (2.7%) Spitz nevi, 1 of 32 (3.1%) atypical Spitz tumors, and 1 of 33 (3%) spitzoid melanoma (FIG. 6a). Fusions of RET on chromosome 10q11 involved the 5′ fusion partners KIF5B on chromosome 10p11 and GOLGA45 on chromosome 14q32 (FIG. 2b). In both fusions, the RET tyrosine kinase domain was fused to the coiled-coil domains of KIF5B (exons 1-16) or GOLGA5 (exons 1-7). Immunohistochemical expression of RET was observed only in cases with RET translocations (FIG. 6b). Expression of the GOLGA5-RET fusion construct in melan-a cells showed increased phosphorylation of the fusion protein, AKT, ERK, S6, and PLCγ-1 compared to control cells. The phosphorylation of these proteins could be suppressed by vandetanib (FIG. 6c) or cabozantinib (FIG. 6d), which are both small molecule RET inhibitors that are FDA approved for medullary thyroid cancer.

BRAF Fusions:

Gene rearrangements of the serine/threonine kinase BRAF were found in 4 of 75 (5.3%) Spitz nevi, 2 of 32 (6.3%) atypical Spitz tumors, and 1 of 33 (3%) spitzoid melanoma. The fusion genes contained CEP89 exons 1-16 followed by the kinase domain of BRAF encoded by exons 9-18, or LSM14A exons 1-9 followed by BRAF exons 9-18, both resulting in loss of the auto-inhibitory, N-terminal RAS-binding domain of BRAF (FIG. 2b). In addition to the identified translocations, 1 of 75 (1.3%) Spitz nevi and 1 of 32 (3.1%) atypical Spitz tumors showed BRAF amplification as determined by at least 8 BRAF fusion signals per nucleus in interphase FISH.

These findings of the activated effector pathways downstream of the fusion kinases are in line with other experimental systems of cancer. For example, ubiquitous expression of constitutively active RFP-RET fusions in mice results in generalized melanocyte proliferation, formation of nevi and ultimately melanomas with MAPK pathway activation; this is similar to the finding of GOLGA5-RET fusions in spitzoid neoplasms (Kato, M. et al. Oncogene 17, 1885-8 (1998)). Autocrine neurotrophin signaling involving NTRK1 has been shown to promote proliferation and migration in melanoma cell lines (Truzzi, F. et al. J Invest Dermatol 128, 2031-40 (2008)), and constitutively active NTRK1 activates MAPK and PI3K pathways in NIH-3T3 cells (Reuther, G. W. et al. Mol Cell Biol 20, 8655-66 (2000)). Phosphorylation of the MAPK and PI3K pathways is an essential component of oncogenic ALK signaling in lymphoma (Lamant, L., et al., Blood 93, 3088-95 (1999)) and lung cancer (Soda, M. et al. Nature 448, 561-6 (2007)). The novel fusion DCTN1-ALK has similar effects. The activation of the MAPK and PI3K pathways by the novel PWWP2A-ROS1 fusion is also similar to the changes associated with other oncogenic ROS1 fusions in various cancer types (Bergethon, K. et al. J Clin Oncol 30, 863-70 (2012); Jun, H. J. et al. Cancer Res 72, 3764-74 (2012)). The results of the present functional validation experiments in xenograft models are consistent with previous studies which showed that TPM3-ROS1 (Takeuchi, K. et al. Nat Med 18, 378-81 (2012)), EML4-ALK (Soda, M. et al. Nature 448, 561-6 (2007)), KIF5B-RET (Lipson. D. et al. Nat Med 18, 382-4 (2012); Takeuchi. K. et al. Nat Med 18, 378-81 (2012); Kohno, T. et al. Nat Med 18, 375-7 (2012)) fusions drive tumor formation in non-small cell lung cancers.

The common occurrence of kinase fusions in the entire biologic spectrum of spitzoid neoplasms (benign Spitz nevi, atypical Spitz tumors, spitzoid melanomas) suggests that these fusions occur early in the pathogenesis of spitzoid neoplasms. Therefore, kinase fusions are necessary, but not sufficient for malignant transformation, a situation analogous to that of mutations in oncogenes (such as BRAF, NRAS, GNAQ and GNA11) commonly found in melanocytic neoplasms. Consequently, the frequent kinase fusions described herein are unlikely to be useful in distinguishing benign from malignant spitzoid neoplasms.

Although the majority of spitzoid neoplasms behave in an indolent fashion, some spitzoid neoplasms metastasize and require systemic therapy. Lung cancers, lymphomas, and sarcomas with ALK or ROS1 fusions, and thyroid cancers with RET fusions can be successfully treated using US Food and Drug Administration (FDA)-approved kinase inhibitors such as crizotinib, cabozantinib, and vandetanib. The fusion kinases identified here are therefore potential therapeutic targets for translocation-positive melanocytic tumors that require systemic therapy.

TABLE 9
Plasmids used for the fusion constructs

| | | |
|---|---|---|
| LaminA | NM_170707.2 | pBABE-puro-GFP-wt-lamin A |
| | | (Addgene plasmid 17662) |
| NTRK1 | NM_001012331.1 | pCMV5-TrkA (Addgene plasmid |
| | | 15002) |
| Golga5 | NM_005113.2 | pCMV6-XL5-Golga5 (Origene |
| | | plasmid SC116934) |
| RET | NM_020975.4 | pGEM-T-Ret (Sino Biological |
| | | plasmid HG11997-G) |
| ROS1 | NM_002944.2 | pENTR223.1-Ros1 (Dana Farber/ |
| | | Harvard Cancer Center DNA Resource |
| | | Core plasmid HsCD00294952) |
| PWWP2A | NM_052927.2 | pCR4-TOPO-PWWP2A (Dana Farber/ |
| | | Harvard Cancer Center DNA Resource |
| | | Core plasmid HsCD00341819) |
| ALK | NM_004304.4 | pDONR223-ALK (Addgene plasmid |
| | | 23917) |
| DCTN1 | NM_004082.4 | pCMV6-XL4 DCTN1 (Origene plasmid |
| | | SC110869) |

TABLE 11
Genomic positions of 5′ and 3′ fusion proteins.

| | | | | |
|---|---|---|---|---|
| Gene | | Genomic position* | Genomic position | Genomic position |
| name | Accession # | (full gene) | (fused portion) | (non-fused portion) |

5′ fusion genes

| | | | | |
|---|---|---|---|---|
| CLIP1 | NM_002956 | chr12: 122755981- | chr12: 122772967- | — |
| | | 122907116 | 122907116 | |
| PPFIBP1 | NM_003622 | chr12: 27677045- | chr12: 27677045- | — |
| | | 27848497 | 27809744 | |
| TPM3 | NM_152263 | chr1: 154134289- | chr1: 154148512- | — |
| | | 154164611 | 154164611 | |
| ZCCHC8 | NM_017612 | chr12: 122956146- | chr12: 122985121- | — |
| | | 122985620 | 122985620 | |
| MYO5A | NM_000259 | Chr15: 52599480- | chr15: 52659148- | — |
| | | 52821247 | 52821247 | |
| PWWP2A | NM_052927 | chr5: 159502892- | chr5: 159545753- | — |
| | | 159546452 | 159546452 | |
| GOLGA5 | NM_005113 | chr14: 93260576- | chr14: 93260576- | — |
| | | 93306306 | 93282825 | |
| KIF5B | NM_004521.2 | chr10: 32297938- | chr10: 32311722- | — |
| | | 32345371 | 32345371 | |
| TP53 | NM_001126113 | chr17: 7571720- | chr17: 7571720- | — |
| | | 7590868 | 7571669 | |
| CEP89 | NM_032816 | chr19: 33369904- | chr19: 33390670- | — |
| | | 33462869 | 33462869 | |
| HLA-A | NM_002116 | chr6: 29910247- | chr6: 29910247- | — |
| | | 29913661 | 29913146 | |
| ERC1 | NM_178039 | chr12: 1100404- | chr12: 1100404- | — |
| | | 1605099 | 1299303 | |
| KIAA1598 | NM_001127211 | chr10: 118642888- | chr10: 118687239- | — |
| | | 118765088 | 118765088 | |
| DCTN1 | NM_004082 | chr2: 74588281- | chr2: 74592133- | — |
| | | 74607482 | 74607482 | |
| LSM14A | NM_001114093 | chr19: 34663352- | chr19: 34663352- | — |
| | | 34720420 | 34712701 | |
| LMNA | NM_005572 | chr1: 156084461- | chr1: 156084461- | — |
| | | 156107657 | 156100660 | |
| FMN1 | NM_001277313 | chr15: 33057745- | chr15: 33090935- | — |
| | | 33486934 | 33486934 | |

3′ fusion kinases

| | | | | |
|---|---|---|---|---|
| ROS1 | NM_002944 | chr6: 117609530- | chr6: 117609530- | chr6: 117645619- |
| | | 117747018 | 117645619 | 117747018 |
| ALK | NM_004304 | chr2: 29415640- | chr2: 29415640- | chr2: 29446428- |
| | | 30144477 | 29446428 | 30144477 |
| RET | NM_020975 | chr10: 43572517- | chr10: 43612016- | chr10: 43572517- |
| | | 43625797 | 43625797 | 43612016 |
| NTRK1 | NM_002529 | chr1: 156830671- | chr1: 156844170- | chr1: 156830671- |
| | | 156851642 | 156851642 | 156844170 |
| BRAF | NM_004333 | chr7: 140433813- | chr7: 140433813- | chr7: 140487415- |
| | | 140624564 | 140487415 | 140624564 |

*Predicted genomic position from genome database Human February 2009 (GRCh37/hg19) Assembly on UCSC Genome Browser.

### Example 3

### Functional Validation Studies of Kinase Fusion Proteins in Xenograft Models

To validate the oncogenic roles of the novel fusion kinases, mouse melan-a cells were stably transduced with PWWP2A-ROS1, DCTN1-ALK, LMNA-NTRK1, GOLGA5-RET fusions. The transduced cells were then injected subcutaneously and bilaterally into the flank of immunocompromised mice. 1.5 million melan-a cells stably transduced with the indicated oncogene or GFP control vector were injected subcutaneously in two separate, 6 week old female NOD-scid, IL2-Rγ^{null} mice. Mice were palpated 3 times a week for the development of tumors over a period of 40 days.

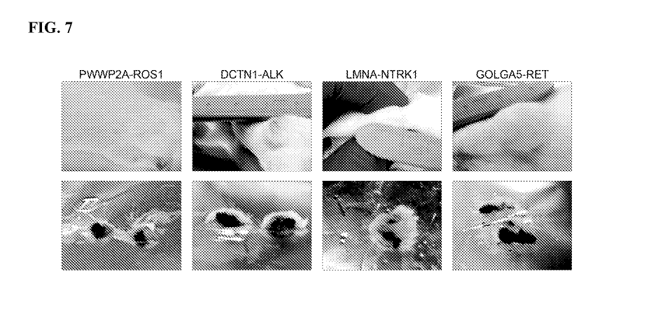

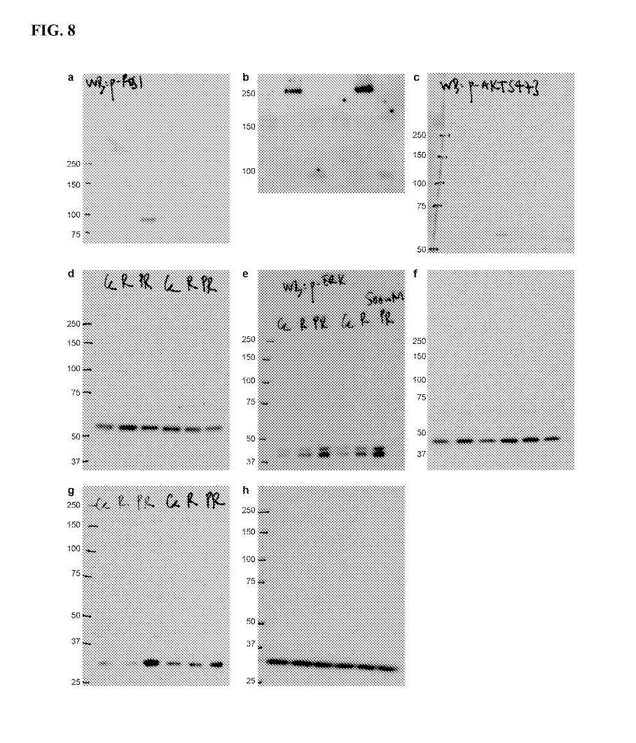

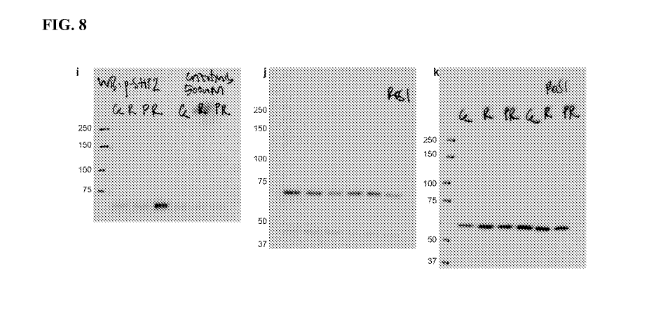

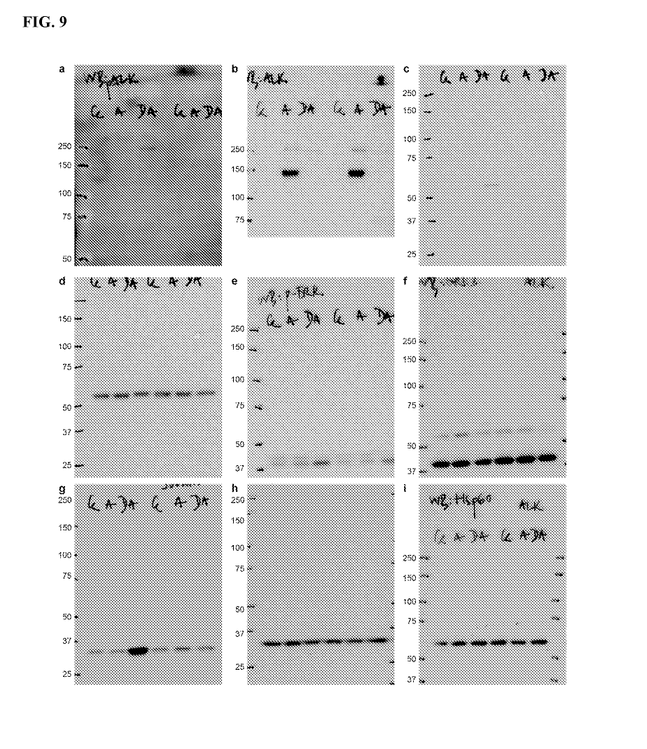

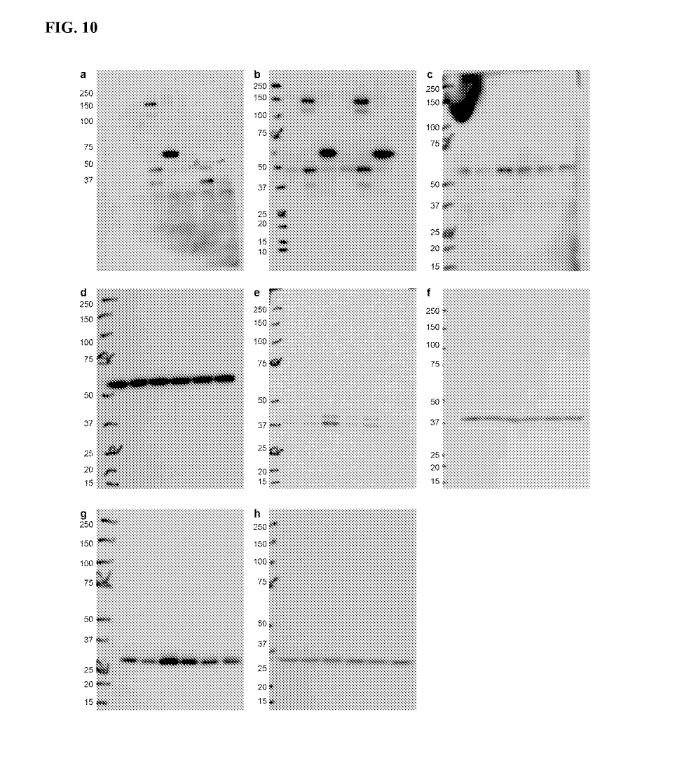

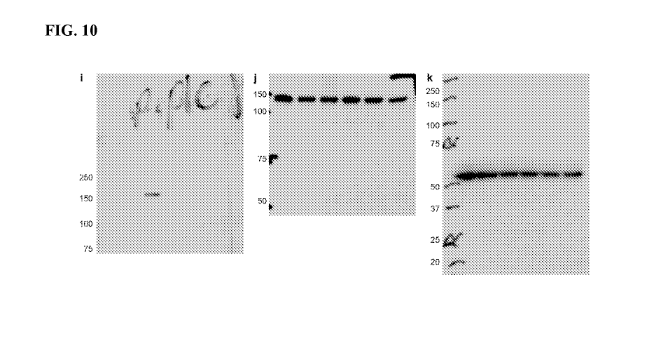

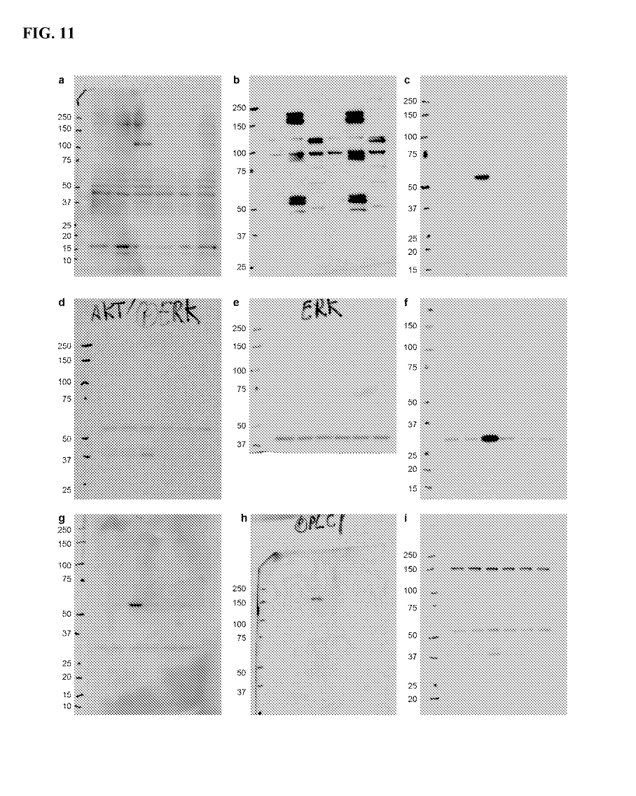

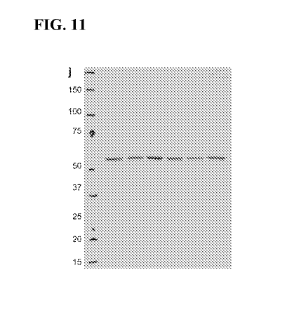

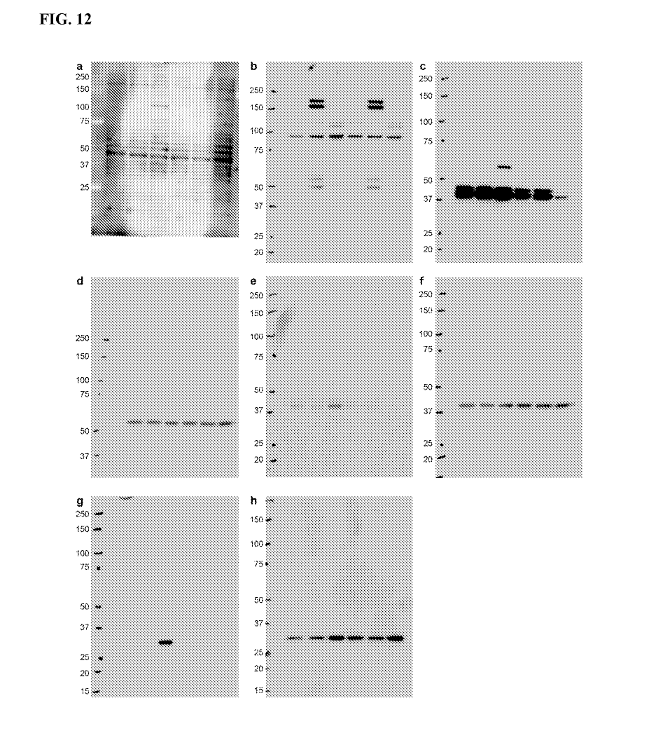

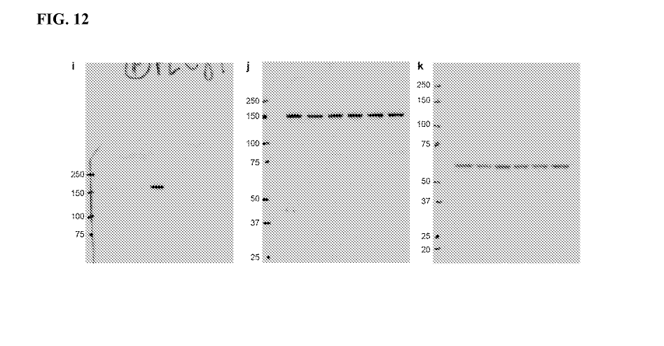

Mice were monitored for tumor formation every 3 to 4 days. Within 40 days, all injection sites in the fusion kinase groups (PWWP2A-ROS1, DCTN1-ALK, LMNA-NTRK1, GOLGA5-RET) developed rapidly growing tumors similarly to the one observed at the injection sites of melanocytes expressing the known melanoma oncogenes HRAS^{G12V} and NRAS^{G12V} (FIG. 7). No tumors were observed at the injection sites in the control group with melanocytes that had been transduced with GFP (FIG. 7).

The results of the foregoing functional validation experiments in xenograft models are consistent with previous studies which showed that TPM3-ROS1 (Takeuchi, K. et al. Nat Med 18, 378-81 (2012)), EML4-ALK (Soda, M. et al. Nature 448, 561-6 (2007)), KIF5B-RET (Lipson, D. et al. Nat Med 18, 382-4 (2012); Takeuchi, K. et al. Nat Med 18, 378-81 (2012); Kohno, T. et al. Nat Med 18, 375-7 (2012)) fusions drive tumor formation in non-small cell lung cancers.

### ADDITIONAL DISCLOSURE

A. A use of a kinase inhibitor for treating melanoma in a patient afflicted with melanoma exhibiting a melanocytic neoplasm having one or more nucleic acid translocations, each nucleic acid translocation coding for a fusion protein comprising a portion of a kinase comprising a kinase domain, wherein the fusion protein has enhanced kinase activity as compared to a kinase in a control sample without the translocation.
B. A method for treating melanoma in a patient afflicted with melanoma and exhibiting melanocytic neoplasm having one or more nucleic acid translocations, each nucleic acid translocation coding for a fusion protein comprising a portion of a kinase comprising a kinase domain, the method comprising:
   administering to the patient an effective amount of a kinase inhibitor that inhibits the kinase, the kinase domain of which is a constituent of said fusion protein.
C. A use or a method of determining whether a patient exhibiting melanocytic neoplasm is a candidate for treatment with a kinase inhibitor comprising:
   translocations in a lesion of the patient with a first reagent, each nucleic acid translocation coding for a fusion protein comprising a portion of a kinase comprising a kinase domain.
D. The method or use of any one of Paragraphs A-C, wherein the fusion protein further comprises a portion of a fusion partner.
E. The method or use of any one of Paragraphs A-D, wherein the kinase portion in the fusion protein lacks all or a portion of an extracellular receptor domain.
F. The method or use of any one of Paragraphs A-E, wherein the kinase is selected from the group consisting of ROS1, ALK, BRAF, RET, and NTRK1.
G. The method or use of Paragraph D, wherein the portion of the protein partner comprises a coiled coil domain.
H. The method of Paragraph G, wherein the fusion partner is selected from the group consisting of HLA-A, MYO5A, PPFIBP1, ERC1, PWWP2A, CLIP1, TPM3, KIAA1598, DCTN1, TP53, LMNA, GOLGA, KIF5B, CEP89, FMN1 and LSM14A.
I. The method of Paragraph C, wherein the first reagent is a set of reagents, wherein each reagent in the set detects a different nucleic acid translocation.
J. A composition comprising a set of first reagents, the set comprising a reagent that detects a translocation in a nucleic acid coding for a fusion protein comprising a portion of a ROS1 kinase, a reagent that detects a translocation in a nucleic acid coding for a fusion protein comprising a portion of a ALK kinase, a reagent that detects a translocation in a nucleic acid coding for a fusion protein comprising a portion of a BRAF kinase, a reagent that detects a translocation in a nucleic acid coding for a fusion protein comprising a portion of a RET kinase, and a reagent that detects a translocation in a nucleic acid coding for a fusion protein comprising a portion of a NTRK1 kinase.
K. The composition of Paragraph J, further comprising a second reagent that specifically detects the full length kinase, or detects a portion of the full length kinase that is not containing the kinase domain.
L. The composition of Paragraph J, wherein the second reagent specifically detects all or a portion of another protein partner that the kinase is fused to, wherein the protein partner is selected from the group consisting of HLA-A, MYO5A, PPFIBP1, ERC1, PWWP2A, CLIP1, TPM3, KIAA1598, DCTN1. TP53, LMNA, GOLGA, KIF5B, CEP89, FMN1 and LSM14A.
M. The composition of Paragraph J, wherein the first reagent is a probe that specifically binds to the nucleic acid coding for the fusion protein.
N. The composition of Paragraph J, wherein the first reagent is a probe that specifically binds to a nucleic acid coding for the fusion protein wherein the probes comprise:
   a) at least one 3′ ROS1 probe that hybridizes with all or a portion of a sequence selected from the group consisting of:
   nucleotide position 5757 to position 7243 of SEQ ID NO: 1; or
   b) at least one 3′ ALK probe that hybridizes with all or a portion of a sequence selected from the group consisting of:
   nucleotide position 4125 to position 5815 of SEQ ID NO:7; or
   c) at least one 3′ BRAF probe that hybridizes with all or a portion of a sequence selected from the group consisting of:
   nucleotide position 1202 to position 2362 of SEQ ID NO:1; or
   d) at least one 3′ RET probe that hybridizes with all or a portion of a sequence selected from the group consisting of:
   nucleotide position 2327 to position 3409 of SEQ ID NO:5; or
   e) at least one 3′ NTRK1 probe that hybridizes with all or a portion of a sequence selected from the group consisting of:
   3 and, a nucleotide sequence that is at least 70% identical to any of these sequences.
O. The composition of Paragraph K, wherein the second reagent is a probe that specifically binds to a nucleic acid encoding the full length kinase or specifically binds to a nucleic acid encoding a portion of the full length kinase not containing the kinase domain.
P. The composition of Paragraph K, wherein the second reagent is a probe that specifically binds to a nucleic acid coding for the fusion protein wherein the probes comprise:
   a) at least one 5′ ROS1 probe that hybridizes with all or a portion of a sequence selected from the group consisting of:
   nucleotide position 1 to position 5757 of SEQ ID NO: 11; or
   b) at least one 5′ ALK probe that hybridizes with all or a portion of a sequence selected from the group consisting of:
   nucleotide position 1 to position 4125 of SEQ ID NO:7; or
   c) at least one 5′ BRAF probe that hybridizes with all or a portion of a sequence selected from the group consisting of:
   nucleotide position 1 to position 1202 of SEQ ID NO:1; or
   d) at least one 5′ RET probe that hybridizes with all or a portion of a sequence selected from the group consisting of:
   nucleotide position 1 to position 2327 of SEQ ID NO:5; or
   e) at least one 5′ NTRK1 probe that hybridizes with all or a portion of a sequence selected from the group consisting of:
   nucleotide position 1 to position 1234 of SEQ ID NO:3
   and, a nucleotide sequence that is at least 70% identical to any of these sequences.
Q. The composition of Paragraph L, wherein the second reagent is a probe that specifically binds to a nucleic acid coding for all or a portion of another protein partner that the kinase is fused to, wherein the probes comprise:
   a) at least one 5′ HLA-A probe that hybridizes with all or a portion of a sequence selected from the group consisting of:
   nucleotide position 1 to position 1096 of SEQ ID NO: 37; or
   b) at least one 5′ MYO5A probe that hybridizes with all or a portion of a sequence selected from the group consisting of:
   nucleotide position 1 to position 3404 of SEQ ID NO: 29; or
   c) at least one 5′ PPFIBP1 probe that hybridizes with all or a portion of a sequence selected from the group consisting of:
   nucleotide position 1 to position 2423 of SEQ ID NO:19; or
   d) at least one 5′ ERC1 probe that hybridizes with all or a portion of a sequence selected from the group consisting of:
   nucleotide position 1 to position 2644 of SEQ ID NO: 41; or
   e) at least one 5′ PWWP2A probe that hybridizes with all or a portion of a sequence selected from the group consisting of:
   nucleotide position 1 to position 641 of SEQ ID NO: 33; or
   f) at least one 5′ CLIP1 probe that hybridizes with all or a portion of a sequence selected from the group consisting of:
   nucleotide position 1 to position 3887 of SEQ ID NO: 15; or
   g) at least one 5′ TPM3 probe that hybridizes with all or a portion of a sequence selected from the group consisting of:
   nucleotide position 1 to position 494 of SEQ ID NO: 9; or
   h) at least one 5′ KIAA1598 probe that hybridizes with all or a portion of a sequence selected from the group consisting of:
   nucleotide position 1 to position 1610 of SEQ ID NO: 64; or
   i) at least one 5′ DCTN1 probe that hybridizes with all or a portion of a sequence selected from the group consisting of:
   nucleotide position 1 to position 3514 of SEQ ID NO: 66; or
   all or a portion of a sequence selected from the group consisting of: nucleotide position 1 to position 2651 of SEQ ID NO: 62; or
   k) at least one 5′ LMNA probe that hybridizes with all or a portion of a sequence selected from the group consisting of:
   nucleotide position 1 to position 762 of SEQ ID NO: 70; or
   1) at least one 5′ ZCCHC8 probe that hybridizes with all or a portion of a sequence selected from the group consisting of:
   nucleotide position 1 to position 357 of SEQ ID NO: 25; or
   m) at least one 5′ GOLGA probe that hybridizes with all or a portion of a sequence selected from the group consisting of:
   nucleotide position 1 to position 1802 of SEQ ID NO: 47; or
   n) at least one 5′ KIF5B probe that hybridizes with all or a portion of a sequence selected from the group consisting of:
   nucleotide position 1 to position 2384 of SEQ ID NO: 55; or
   o) at least one 5′ CEP89 probe that hybridizes with all or a portion of a sequence selected from the group consisting of:
   nucleotide position 1 to position 1964 of SEQ ID NO: 51; or
   p) at least one 5′ LSM14A probe that hybridizes with all or a portion of a sequence selected from the group consisting of:
   nucleotide position 1 to position 1621 of SEQ ID NO: 68; or
   q) at least one 5′ FMN1 probe that hybridizes with all or a portion of a sequence selected from the group consisting of:
   nucleotide position 1 to position 4634 of SEQ ID NO: 72

and, a nucleotide sequence that is at least 70% identical to any of these sequences.
R. The composition of any one of Paragraph I-F, wherein the kinase ROS1, ALK, BRAF, RET, or NTRK1 is fused with another gene that is not HLA-A, MYO5A, PPFIBP1, ERC1, PWWP2A, CLIP1, TPM3, KIAA1598, DCTN1, TP53, LMNA, GOLGA, KIF5B, CEP89, FMN1 or LSM14A.
S. The composition of any one of Paragraph J-L, wherein the first reagent is a set of primers that specifically amplifies all or a portion of the nucleic acid translocation.
T. The composition of Paragraph J, wherein the set of primers amplify a nucleic acid coding for a fusion junction between the portion of the protein partner and the kinase domain.
U. The composition of Paragraph I or J, wherein the second reagent is a set of primers that specifically amplifies all or a portion of the full length kinase, wherein the portion of the full length kinase does not include the kinase domain.
V. The composition of Paragraph L, wherein the second reagent is a set of primers that specifically amplifies all or a portion of all or a portion of another protein partner that the kinase is fused to, wherein the protein partner is selected from the group consisting of HLA-A, MYO5A, PPFIBP1, ERC1, PWWP2A, CLIP1, TPM3, KIAA1598, DCTN1, TP53, LMNA, GOLGA, KIF5B, CEP89, FMN1 and LSM14A.
W. The composition of any one of Paragraphs A-F, wherein the first reagent is an antibody that specifically binds to the fusion protein.
X. The composition of Paragraph K, wherein the second reagent is an antibody that specifically binds to the full length kinase but not to the fusion protein.
Y. The composition of Paragraph M, wherein the second reagent an antibody that specifically binds to all or a portion of all or a portion of another protein partner that the kinase is fused to, wherein the protein partner is selected from the group consisting of HLA-A, MYO5A, PPFIBP1, ERC1, PWWP2A, CLIP1, TPM3, KIAA1598, DCTN1, TP53, LMNA, GOLGA, KIF5B, CEP89, FMN1 and LSM14A.
Z. The method or use of the composition of any one of Paragraphs J-Y for selecting a patient having a nucleic acid translocation for treatment with a kinase inhibitor.
AA. The method or use of the composition of any one of Paragraphs J-V to detect the presence of a nucleic acid translocation in a sample to identify a Spitz neoplasm.
BB. The method or use of the composition of any one of Paragraphs J-V in a method of treating a patient having melanoma with a kinase inhibitor.
CC. The method or use of Paragraphs Z or AA, wherein the kinase inhibitor is effective to inhibit the kinase domain of the fusion protein.
DD. A method of identifying whether a melanocytic tumor is likely to be responsive to a tyrosine kinase inhibitor or a serine/threonine kinase inhibitor comprising:
   (a) contacting a test sample of the tumor with at least one first reagent that detects a translocation in a nucleic acid molecule encoding a kinase protein, wherein the translocation has been determined to cause enhanced kinase activity in the tumor as compared to the same kinase in a control sample without the translocation;
   (b) if the first reagent produces a positive reading for the translocation, identifying the tumor as likely to respond to a specific tyrosine kinase or serine/threonine kinase inhibitor; and if the first reagent produces a negative reading for the translocation, identifying the tumor as not likely to respond to a specific tyrosine or serine/threonine kinase inhibitor.
EE. Use according to Paragraph A wherein said patient is afflicted with a spitzoid melanoma.
FF. The method or use of Paragraph BB wherein said kinase inhibitor is selected from the group of kinase inhibitors in Table 10.
GG. The method or use of claim 28 wherein the kinase inhibitor is selected from the group consisting of vemurafenib (also known as RG7204; or PLX4032; or Zelboraf); GDC-0879; PLX-4702; AZ628; dabrafenib (also known as GSK2118346); LGX818; BMS-908662, PLX3603, RAF265, RO5185426, trametinib; Sorafenib Tosylate; pyrazolopyrimidine PP1 and PP2; indocarbazole derivative CEP-701 (also known as lestaurtinib) and CEP-751; 2-indolinone RPI-1; quinazoline, ZD6474; and TG101209; vandetanib, cabozantinib; AZ-23; indenopyrrolocarboazole 12a; oxindole 3; isothiazole 5n; thiazole 20h; dasatinib; AZ64; TAE-684 (also known as “NVP-TAE694”), PF02341066 (also known as “crizotinib” or “1066” or Xalkori), LDK-378, ASP-3026, CEP-37440, CEP-28122, CEP-108050, MK-2206, perifosine, sorafenib; AP26113; Ganetespib; 4, X-276, X-376, X-396, CH5424802 (also known as AF-802), GSK1838705, PHA-E429, CRL151104A; alisertib (MLN8237), axitinib (AG013736), bosutinib (SKI-606), cediranib (RECENTIN™, AZD2171), dasatinib (SPRYCEL®, BMS-354825), deforolimus (AP23573/MK-8669), dovitinib lactate (TKI258, CHIR-258), enzastaurin (LY317615), everolimus (RAD011), erlotinib (TARCEVA®), fostamatinib (FosDiR788), gefitinib (IRESSA®), imatinib (Gleevec®, CGP57148B, STI-571), ibrutinib (PCI-32765), lapatinib (TYKERB®, TYVERB®), lestaurtinib (CEP-701), neratinib (HKI-272), nilotinib (TASIGNA®), pacritinib (SB1518), ponatinib (Iclusig), semaxanib (semaxinib, SU5416), sorafenib (NEXAVAR®), sunitinib (SUTENT®, SU11248), temsirolimus (CCI-779/Torisel), tipifarnib (Zarnestra, R115777), tivozanib (AV-951), toceranib (PALLADIA®), vandetanib, vatalanib (PTK787, PTK/ZK), ENMD-2076, PCI-32765, AC220, BIBW 2992 (TOVOK™). SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, OSI-930, MM-121, XL-184, XL-647, LDK378, GS-1101 (CAL-101), MK-2206, perifosine, LGX818, BMS-908662, PLX3603, RAF265, RO5185426, trametinib, cabozantinib, AZ64, AP26113, X-276, X-376, X-396, C115424802 (AF-802), GSK1838705, ASP3026, PHA-E429, CRL151104A and XL228.
HH. An isolated nucleic acid molecule comprising a polynucleotide sequence that is at least 70% identical to a sequence selected from the group consisting of: SEQ ID No: 13, SEQ ID No:92, SEQ ID No:17, SEQ ID No:86, SEQ ID No:21, SEQ ID No:94, SEQ ID No:23, SEQ ID No:96, SEQ ID No:27, SEQ ID No:84, SEQ ID No:31, SEQ ID No:90, SEQ ID No:43, SEQ ID No:98, SEQ ID No:45, SEQ ID No: 102, SEQ ID No:53, SEQ ID No: 100, SEQ ID No:57, SEQ ID No:106, SEQ ID No:59, SEQ ID No:107, SEQ ID No:60, SEQ ID No:108, SEQ ID No:61, SEQ ID No: 109, SEQ ID No:49, SEQ ID No: 104, SEQ ID No:35, SEQ ID No:82, SEQ ID No:39, SEQ ID No:88, SEQ ID No:74, SEQ ID No:76, SEQ ID No:78, SEQ ID No:80, SEQ ID No:110, or SEQ ID No: 112.
II. An isolated polypeptide molecule comprising a polypeptide sequence that is at least 70% identical to a sequence selected from the group consisting of: SEQ ID NO: 14, SEQ ID NO:93, SEQ ID NO: 18, SEQ ID NO:87, SEQ ID NO:22, SEQ ID NO:95, SEQ ID NO:24, SEQ ID NO:97, SEQ ID NO:28, SEQ ID NO:85, SEQ ID NO:32, SEQ ID NO:91, SEQ ID NO:44, SEQ ID NO:99, SEQ ID NO:46, SEQ ID NO: 103, SEQ ID NO:54, SEQ ID NO: 101, SEQ ID NO:58, SEQ ID NO:50, SEQ ID NO:105, SEQ ID NO:36, SEQ ID NO:83, SEQ ID NO:40, SEQ ID NO:89, SEQ ID NO:75, SEQ ID NO:77, SEQ ID NO:79, SEQ ID NO:81, SEQ ID NO:111, OR SEQ ID NO:113.
JJ. The composition of Paragraph J, wherein the first reagent is a set of probes that specifically bind to a nucleic acid wherein the probes comprise:

a) at least one 3′ ROS1 probe that hybridizes to all or a portion of a genomic sequence selected from chromosome 6 position 117609530 to position 117645619 (Genome Reference Consortium Human Build 37, i.e., GRCh37/hg19);
b) at least one 3′ ALK probe that hybridizes with all or a portion of a genomic sequence selected from chromosome 2 position 29415640 to position 29446428 (GRCh37/hg19);
c) at least one 3′ BRAF probe that hybridizes with all or a portion of a genomic sequence selected from chromosome 7 position 140433813 to position 140487415 (GRCh37/hg19);
d) at least one 3′ RET probe that hybridizes with all or a portion of a genomic sequence selected from chromosome 10 position 43612016 to position 43625797 (GRCh37/hg19); or
3′ NTRK1 probe that hybridizes with all or a portion of a genomic sequence selected from chromosome 1 position 156844170 to position 156851642 (GRCh37/hg19 and, a nucleotide sequence that is at least 70% identical to any of these sequences.
KK. The composition of Paragraph K, wherein the second reagent is a probe that specifically binds to a nucleic acid wherein the probes comprise:

b) at least one 5′ ROS1 probe that hybridizes with all or a portion of a genomic sequence selected from chromosome 6 position 117645619 to position 117747018 (GRCh37/hg19)
b) at least one 5′ ALK probe that hybridizes with all or a portion of a genomic sequence selected from chromosome 2 position 29446428 to position 30144477 (GRCh37/hg19);
c) at least one 5′ BRAF probe that hybridizes with all or a portion of a genomic sequence selected from chromosome 7 position 140487415 to position 140624564 (GRCh37/hg19);
d) at least one 5′ RET probe that hybridizes with all or a portion of a genomic sequence selected from chromosome 10 position 43572517 to position 43612016 (GRCh37/hg19); or
(GRCh37/hg19) and, a nucleotide sequence that is at least 70% identical to any of these sequences.
LL. The composition of Paragraph L, wherein the second reagent is a probe that specifically binds to a nucleic acid wherein the probes comprise:
   a) at least one 5′ HLA-A probe that hybridizes with all or a portion of a sequence selected from chromosome 6 position 29910247 to position 29913146 (GRCh37/hg19);
   b) at least one 5′ MYO5A probe that hybridizes with all or a portion of a sequence selected from chromosome 15 position 52659148 to position 52821247 (GRCh37/hg19);
   c) at least one 5′ PPFIBP1 probe that hybridizes with all or a portion of a sequence selected from chromosome 12 position 27677045 to position 27809744 (GRCh37/hg19);
   d) at least one 5′ ERC1 probe that hybridizes with all or a portion of a sequence selected from chromosome 12 position 1100404 to position 1299303 (GRCh37/hg19);
   e) at least one 5′ PWWP2A probe that hybridizes with all or a portion of a sequence selected from chromosome 5 position 159545753 to position 159546452 (GRCh37/hg19);

f) at least one 5′ CLIP1 probe that hybridizes with all or a portion of a sequence selected from chromosome 12 position 122772967 to position 122907116 (GRCh37/hg19);
g) at least one 5′ TPM3 probe that hybridizes with all or a portion of a sequence selected from chromosome 1 position 154148512 to position 154164611 (GRCh37/hg19);
h) at least one 5′ KIAA1598 probe that hybridizes with all or a portion of a sequence selected from chromosome 10 position 118687239 to position 118765088 (GRCh37/hg19);
i) at least one 5′ DCTN1 probe that hybridizes with all or a portion of a sequence selected from chromosome 2 position 74592133 to position 74607482 (GRCh37/hg19);
j) at least one 5′ TP53 probe that hybridizes with all or a portion of a sequence selected from chromosome 17 position 7571720 to position 7571669 (GRCh37/hg19);
k) at least one 5′ LMNA probe that hybridizes with all or a portion of a sequence selected from chromosome 1 position 156084461 to position 156100660 (GRCh37/hg19);
l) at least one 5′ ZCCHC8 probe that hybridizes with all or a portion of a sequence selected from chromosome 12 position 122985121 to position 122985620 (GRCh37 hg19);
m) at least one 5′ GOLGA probe that hybridizes with all or a portion of a sequence selected from chromosome 14 position 93260576 to position 93282825 (GRCh37/hg19);
n) at least one 5′ KIF5B probe that hybridizes with all or a portion of a sequence selected from chromosome 10 position 32311722 to position 32345371 (GRCh37/hg19):
o) at least one 5′ CEP89 probe that hybridizes with all or a portion of a sequence selected from chromosome 19 position 33390670 to position 33462869 (GRCh37/hg19);
p) at least one 5′ LSM14A probe that hybridizes with all or a portion of a sequence selected from chromosome 19 position 34663352 to position 34712701 (GRCh37/hg19); or
all or a portion of a sequence selected from chromosome 15 position 33090935 to position 33486934 (GRCh37 hg19 and, a nucleotide sequence that is at least 70% identical to any of these sequences.

### EQUIVALENTS

Those skilled in the art will recognize, or be able to ascertain using no more than routine experimentation, many equivalents to the specific embodiments described herein. Such equivalents are intended to be encompassed by the following claims.

We claim:

1. An isolated or purified nucleic acid molecule that encodes a fusion, or a breakpoint comprising fragment thereof, chosen from CLIP1-ROS1; PPFIBP1-ROS1; TPM3-ROS1; ZCCHC8-ROS1; MYO5A-ROS1; PWWP2A-ROS1; HLA-A-ROS1; ERC1-ROS1; TPM3-ALK; GOLGA5-RET; CEP89-BRAF; KIF5B-RET; or TP53-NTRK1, summarized in FIG. 1A-1C, or a sequence at least 85% identical thereto.

2. A nucleic acid molecule that is capable of hybridizing to a fusion comprising the nucleotide sequence of CLIP1-ROS1; PPFIBP1-ROS1; TPM3-ROS1; ZCCHC8-ROS1; MYO5A-ROS1; PWWP2A-ROS1; HLA-A-ROS1; ERC1-ROS1; TPM3-ALK; GOLGA5-RET; CEP89-BRAF; KIF5B-RET; or TP53-NTRK1, summarized in FIG. 1A-1C, or a fragment thereof comprising a breakpoint.

3. A fragment of the nucleic acid molecule of either of claims 1-2, wherein said fragment comprises oligonucleotides between 10 and 25 nucleotides in length, or between 100 to 300 nucleotides in length.

4. The fragment of claim 3, which is a probe or primer that includes an oligonucleotide between about 5 and 25 nucleotides in length.

5. The fragment of claim 3, which is a bait that comprises an oligonucleotide between about 100 to 300 nucleotides, 130 and 230 nucleotides, or 150 and 200 nucleotides, in length.

6. A nucleic acid molecule of any of claims 1-5 suitable as probe, primer, bait or library member that specifically binds to the fusion.

7. The isolated or purified nucleic acid molecule of any of claims 1-5, which is operatively linked to a native or a heterologous regulatory sequence.

8. An isolated or purified vector comprising a nucleic acid molecule of any of claims 1-5.

9. A host cell comprising a vector of claim 8.

10. A nucleic acid molecule that specifically reduces or inhibits the expression of the nucleic acid molecule of any of claims 1-2.

11. The nucleic acid molecule of claim 10, which is chosen from an antisense molecule, ribozyme, siRNA, or triple helix molecule.

12. An isolated or purified fusion chosen from CLIP1-ROS1; PPFIBP1-ROS1; TPM3-ROS1; ZCCHC8-ROS1; MYO5A-ROS1; PWWP2A-ROS1; HLA-A-ROS1; ERC1-ROS1; TPM3-ALK; GOLGA5-RET; CEP89-BRAF; KIF5B-RET; or TP53-NTRK1, summarized in FIG. 1A-1C, or a fragment thereof, or a sequence at least 85% identical thereto.

13. The isolated or purified fusion polypeptide of claim 12, having a kinase activity, and/or a dimerizing or multimerizing activity.

14. An isolated or purified antibody molecule that specifically binds to the fusion polypeptide of claims 12-13.

15. A reaction mixture comprising:
a detection reagent, or purified or isolated preparation thereof; and
a target nucleic acid derived from a neoplasm or a cancer,
wherein said detection reagent can distinguish a reference sequence from a mutation chosen from: a nucleic acid, or amino acid sequence, having a breakpoint according to FIG. 1A-1C, or an associated mutation.

16. The reaction mixture of claim 15, wherein the detection reagent specifically distinguishes a wild type or another fusion from the fusion nucleic acid.

17. The reaction mixture of claims 15-16, wherein the detection reagent comprises a DNA, RNA or mixed DNA/RNA, molecule which is complementary with a nucleic acid sequence on a target nucleic acid (the detection reagent binding site) wherein the detection reagent binding site is disposed in relationship to the interrogation position such that binding of the detection reagent to the detection reagent binding site allows differentiation of mutant and reference sequences for the mutant.

18. The reaction mixture of any of claims 15-17, wherein the target nucleic acid is from a cancer listed in FIG. 1A, and the detection reagent detects a mutant, e.g., a rearrangement, fusion junction, or fusion of two genes disclosed in FIG. 1A, 1B or 1C.

19. The reaction mixture of claim 18, wherein the target nucleic acid is chosen from one or more:
(i) from a cancer, e.g., a cancer as described herein, and the detection reagent is one that detects a fusion of the CLIP1 and ROS1 genes, e.g., a detection reagent that detects a mutant, e.g., a rearrangement or fusion junction described in FIG. 1A, 1B or 1C or in the section herein entitled Nucleic Acid Molecules, for a fusion of CLIP1 and ROS1;
(ii) from a cancer, e.g., a cancer as described herein, and the detection reagent is one that detects a fusion of the PPFIBP1 and ROS1 genes, e.g., a detection reagent that detects a mutant, e.g., a rearrangement or fusion junction described in FIG. 1A, 1B or 1C or in the section herein entitled Nucleic Acid Molecules, for a fusion of PPFIBP1 and ROS1;
(iii) from cancer, e.g., a cancer as described herein, and the detection reagent is one that detects a fusion of the TPM3 and ROS1 genes, e.g., a detection reagent that detects a mutant, e.g., a rearrangement or fusion junction described in FIG. 1A, 1B or 1C or in the section herein entitled Nucleic Acid Molecules, for a fusion of TPM3 and ROS1;
(iv) from a cancer, e.g., a cancer as described herein, and the detection reagent is one that detects a fusion of the ZCCHC8 and ROS1 genes, e.g., a detection reagent that detects a mutant, e.g., a rearrangement or fusion junction described in FIG. 1A, 1B or 1C or in the section herein entitled Nucleic Acid Molecules, for a fusion of ZCCHC8 and ROS1;
(v) from a cancer, e.g., a cancer as described herein, and the detection reagent is one that detects a fusion of the MYO5A and ROS1 genes, e.g., a detection reagent that detects a mutant, e.g., a rearrangement or fusion junction described in FIG. 1A, 1B or 1C or in the section herein entitled Nucleic Acid Molecules, for a fusion of MYO5A and ROS1;
(vi) from a cancer, e.g., a cancer as described herein, and the detection reagent is one that detects a fusion of the PWWP2A and ROS1 genes, e.g., a detection reagent that detects a mutant, e.g., a rearrangement or fusion junction described in FIG. 1A, 1B or 1C or in the section herein entitled Nucleic Acid Molecules, for a fusion of PWWP2A and ROS1;
(vii) from a cancer, e.g., a cancer as described herein, and the detection reagent is one that detects a fusion of the HLA-A and ROS1 genes, e.g., a detection reagent that detects a mutant, e.g., a rearrangement or fusion junction described in FIG. 1A, 1B or 1C or in the section herein entitled Nucleic Acid Molecules, for a fusion of HLA-A and ROS1;
(viii) from a cancer, e.g., a cancer as described herein, and the detection reagent is one that detects a fusion of the ERC1 and ROS1 genes, e.g., a detection reagent that detects a mutant, e.g., a rearrangement or fusion junction described in FIG. 1A, 1B or 1C or in the section herein entitled Nucleic Acid Molecules, for a fusion of ERC1 and ROS1;
(ix) from a cancer, e.g., a cancer as described herein, and the detection reagent is one that detects a fusion of the TPM3 and ALK genes, e.g., a detection reagent that detects a mutant, e.g., a rearrangement or fusion junction described in FIG. 1A, 1B or 1C or in the section herein entitled Nucleic Acid Molecules, for a fusion of TPM3 and ALK;
(x) from a cancer, e.g., a cancer as described herein, and the detection reagent is one that detects a fusion of the GOLGA5 and RET genes, e.g., a detection reagent that detects a mutant, e.g., a rearrangement or fusion junction described in FIG. 1A, 1B or 1C or in the section herein entitled Nucleic Acid Molecules, for a fusion of GOLGA5 and RET;
(xi) from a cancer, e.g., a cancer as described herein, and the detection reagent is one that detects a fusion of the KIF5B and RET genes, e.g., a detection reagent that detects a mutant, e.g., a rearrangement or fusion junction described in FIG. 1A, 1B or 1C or in the section herein entitled Nucleic Acid Molecules, for a fusion of KIF5B and RET;
(xii) from a cancer, e.g., a cancer as described herein, and the detection reagent is one that detects a fusion of the TP53 and NTRK1 genes, e.g., a detection reagent that detects a mutant, e.g., a rearrangement or fusion junction described in FIG. 1A, 1B or 1C or in the section herein entitled Nucleic Acid Molecules, for a fusion of TP53 and NTRK1; or
(xiii) from a cancer, e.g., a cancer as described herein, and the detection reagent is one that detects a fusion of the CEP89 and BRAF genes, e.g., a detection reagent that detects a mutant, e.g., a rearrangement or fusion junction described in FIG. 1A, 1B or 1C or in the section herein entitled Nucleic Acid Molecules, for a fusion of CEP89 and BRAF.

20. A method of making a reaction mixture comprising:
combining a detection reagent, or purified or isolated preparation thereof, with a target nucleic acid derived from neoplasm or cancer of claim 19, wherein said detection reagent can distinguish a reference sequence from a mutation described herein, or an associated mutation.

21. A purified or isolated preparation of a fusion nucleic acid molecule from a neoplasm or cancer disposed in a sequencing device, or a sample holder for use in such a device, wherein said mutation described herein, or an associated mutation.

22. A purified or isolated preparation of a fusion nucleic acid molecule from a neoplasm or cancer disposed in a device for determining a physical or chemical property, e.g., stability of a duplex, e.g., Tₘ or a sample holder for use in such a device, wherein said, wherein said mutation is described herein, or an associated mutation.

23. A detection reagent comprising a DNA, RNA or mixed DNA/RNA molecule, comprising a nucleotide sequence which is complementary with a nucleic acid sequence on a target nucleic acid in which the detection reagent binding site is disposed in relationship to the interrogation position such that binding (or in embodiments, lack of binding) of the detection reagent to the detection reagent binding site allows differentiation of a mutant and a reference sequence and said target nucleic acid is derived from a neoplasm or cancer,
wherein said mutation described herein or an associated mutation.

24. A purified or isolated preparations of a fusion nucleic acid molecule, e.g., DNA, e.g., genomic DNA or cDNA, or RNA, containing an interrogation position useful for determining if a mutation is present, wherein said nucleic acid molecule is derived from a neoplasm or cancer, wherein said mutation described herein, or an associated mutation.

25. A reaction mixture, comprising:
a detection reagent, or purified or isolated preparation thereof, e.g., a substrate, e.g., a substrate for phosphorylation or other activity, or an antibody, and
a target fusion protein derived from a neoplasm or a cancer, wherein the detection reagent is specific for a fusion described herein, e.g., as summarized in FIGS. 1A-1C.

26. A method of making a reaction mixture comprising:
combining a detection reagent, or purified or isolated preparation thereof, e.g., a substrate, e.g., a substrate for phosphorylation or other activity, or an antibody, described herein with a target fusion protein derived from a neoplasm or cancer, wherein the detection reagent is specific for a fusion described herein e.g., as summarized in FIGS. 1A-1C.

27. A kit comprising a detection reagent of claim 23.
